(19) United States
(12) Patent Application Publication
(10) Pub. No.: US 2022/0411780 A1
(43) Pub. Date: Dec. 29, 2022

(54) DETECTION OF CIRCULATING TUMOR DNA USING DOUBLE STRANDED HYBRID CAPTURE

(71) Applicant: UNIVERSITY HEALTH NETWORK, Toronto (CA)

(72) Inventors: Scott BRATMAN, Toronto (CA); Kathy HAN, Toronto (CA); Zhen ZHAO, Toronto (CA); Jinfeng ZOU, Richmond Hill (CA)

(21) Appl. No.: 17/642,640

(22) PCT Filed: Sep. 11, 2020
(86) PCT No.: PCT/CA2020/051233

§ 371 Date: Mar. 11, 2022

Related U.S. Application Data
(60) Provisional application No. 62/900,237, filed on Sep. 13, 2019.

(51) Int. Cl. C12N 15/10; C12Q 1/6886; C12Q 1/70

(52) U.S. Cl. CPC C12N 15/10; C12Q 1/6886; C12Q 1/708; C12Q 2600/156

(57) ABSTRACT

There is described herein a method for capturing circulating tumor DNA (ctDNA) of interest from an animal sample, preferably a mammalian sample, further preferably a human patient sample, comprising cell-free DNA (cfDNA), the method comprising: adding to the patient sample a library of nucleic acid hybrid capture probes, wherein the library of 5 probes is complementary to both strands of the double stranded ctDNA of interest and the probes are tagged for capture; allowing the probes to hybridize to the ctDNA; and capturing the hybridized ctDNA using the tag on the probes. Libraries of probes for use with these methods are also described.

### RELATED APPLICATIONS

This application claims priority to U.S. Provisional Application No. 62/900,237, filed on Sep. 13, 2019, which is incorporated herein by reference in its entirety.

### FIELD OF THE INVENTION

The invention relates to the detection of circulating tumor DNA, for example the detection of HPV circulating tumor DNA using hybrid capture.

### BACKGROUND OF THE INVENTION

Recent methodological developments based on high throughput sequencing have expanded the potential applications for circulating tumor (ct)DNA analysis^{1,2}. Many clinical settings—particularly those involving patients with low tumor burden—rely on accurate detection of minute levels of ctDNA within a much more abundant pool of cell-free (cf)DNA fragments. Further improvements in low-level ctDNA detection/analysis will have a tremendous impact on future cancer diagnostics for screening, prognostication, and treatment monitoring.

To drive ultrasensitive detection of ctDNA, we and others have developed approaches to increase the number of cancer-specific markers that can be simultaneously assessed in a given patient. By doing so, simulated data has suggested that dramatic gains may be achieved in the probability of detecting <1 genome equivalent (GE; see Methods) of ctDNA. This threshold has been shown to be clinically meaningful in low-disease-burden settings and can therefore be viewed as an important benchmark for ctDNA technological development.

Cancer types that are driven by oncogenic viruses, such as human papillomavirus (HPV)-associated cancers of the cervix and oropharynx, represent approximately 15% of the global cancer burden. In such cancers, the viral genome can be leveraged to distinguish ctDNA from other cellular sources of cfDNA¹⁰⁻²⁰. Most previous efforts have achieved this through quantitative or digital polymerase chain reaction (qPCR or dPCR)^{21,22}, but robust detection of <1 GE has not been observed using these methods^{11,12,17,22,23}. Viral genome hybrid capture sequencing has also recently shown promise as a means for ctDNA quantification while simultaneously providing qualitative information regarding sequenced cfDNA fragments, such as fragment length, that may improve the specificity of ctDNA detection.

### SUMMARY OF THE INVENTION

Robust detection of circulating tumor (ct)DNA can play a role in cancer screening and measurement of minimal residual disease but has remained a challenge in these low-disease-burden settings. Here, we present a new approach for enhancing the recovery of low-level ctDNA by hybrid capture deep sequencing in human papillomavirus (HPV)-associated cancers. We performed hybridization to both sense and antisense baits tiled across the full-length double-stranded DNA HPV genome. This dual-strand hybrid capture procedure recovered fragments typically lost during standard hybrid capture sequencing, enabling reproducible detection of <1 genome equivalent of HPV ctDNA and 30-fold improved sensitivity over digital PCR. HPV-sequencing revealed qualitative information about ctDNA fragments such as HPV genotype, mapping location along the HPV genome, and ctDNA fragment length distribution, all of which can be utilized to improve the specificity of detection. Our findings will have implications for treatment monitoring of HPV-related cancers and could open the door to new potential clinical applications of HPV ctDNA analysis.

In an aspect, there is provided a method for capturing circulating tumor DNA (ctDNA) of interest from an animal sample, preferably a mammalian sample, further preferably a human patient sample, comprising cell-free DNA (cfDNA), the method comprising: adding to the patient sample a library of nucleic acid hybrid capture probes, wherein the library of probes is complementary to both strands of the double stranded ctDNA of interest and the probes are tagged for capture; allowing the probes to hybridize to the ctDNA; and capturing the hybridized ctDNA using the tag on the probes.

In an aspect, there is provided a library of probes complementary to both strands of a double stranded ctDNA of interest and the probes are tagged for capture.

### BRIEF DESCRIPTION OF FIGURES

These and other features of the preferred embodiments of the invention will become more apparent in the following detailed description in which reference is made to the appended drawings wherein:

FIG. 1: Overview of HPV-seq and dual-strand hybrid capture. (A) HPV-seq conducted on plasma cfDNA is designed to provide quantitative and qualitative information about ctDNA in patients with HPV-associated cancers. In addition to being highly sensitive and quantitative, HPV-seq can report on ctDNA fragment size and HPV genotype. Each full-length viral genome (episome or linearized genome) is expected to yield ˜50 distinct cfDNA fragments. (B) HPV-seq is conducted using hybrid capture sequencing with single-stranded (sense [+] and/or antisense [−]) biotinylated baits tiled across the HPV genome: (i) single strand viral genome hybrid capture; (ii) sequential dual strand hybrid capture; (iii) simultaneous dual strand hybrid capture. (C) Compared with single strand hybrid capture (i), dual strand hybrid capture using either a sequential (ii) or simultaneous (iii) approach recovers more HPV molecules. The degree of HPV DNA enrichment in post-capture libraries was determined using HPV-16 E6 and E7 dPCR assays. N=4 per condition. Error bars represent standard deviation. Asterisk indicates statistical significance (p<0.05) in comparison with single strand capture conditions.

FIG. 2: Analytical sensitivity of HPV-seq. (A) HPV-seq was conducted on fragmented SiHa genomic DNA at the indicated dilution. Hybrid capture baits targeted the indicated HPV-16 sequences. The LLOD of HPV-seq was dependent on the use of dual-strand hybrid capture and the length of HPV-16 genome targeted by the baits. (B) HPV-seq with full-length dual-strand hybrid capture (blue) provided an improvement in analytical sensitivity and LLOD (0.003%) as compared with hybrid capture for a single mutation (1%). (C) Influence of multiple markers and sequencing depth on LLOD. Downsampling of HPV-seq data from full-length dual-strand hybrid capture demonstrates the dependence of the LLOD on the targeted length of HPV-16 genome (i.e., number of markers) (right y-axis) and the sequencing depth (x-axis). The probability of detecting the indicated number of HPV molecules (1—blue circles; 2—gray triangles; 5—yellow squares) is shown (left y-axis).

FIG. 3: HPV ctDNA quantification from plasma cfDNA using HPV-seq. Comparison of dPCR (x-axis) and HPV-seq (y-axis) for 16 cervix cancer patient plasma cfDNA samples obtained at end-of-treatment or post-treatment shows strong correlation. Linear regression and its 95% confidence interval (shaded) are shown.

FIG. 4: End-of-treatment detectable HPV ctDNA is associated with disease recurrence. (A) HPV ctDNA levels at the end-of-treatment timepoint obtained using HPV-seq are significantly higher among patients who subsequently relapsed (N=3) versus patients who remained disease-free (N=5). Horizontal bars indicate the median value and 1.5 times the interquartile range. (B) Progression-free survival according to HPV ctDNA status at the end-of-treatment timepoint. Detectable HPV ctDNA: dashed red line. Undetectable HPV ctDNA: solid blue line. Vertical hash marks indicated censoring.

FIG. 5: Length distributions of human-mapping cfDNA and HPV-mapping ctDNA fragments. (A) Sequenced fragment insert sizes merged from HPV-seq positive patients analyzed at end-of-treatment or post-treatment (N=8). Samples (N=2) from patients with recurrence (B) and samples (N=6) from patients without recurrence (C) display similar ctDNA fragment size distributions. Samples from tumors harboring HPV-16 (N=6) (D) and HPV-33 (N=2) (E) are shown.

FIG. 6: Optimal hybridization conditions for HPV-seq. Dual-strand full-length viral genome hybrid capture was conducted for 72 hr at 47° C. or for 18 hr at 65° C. Per bait concentrations were either 4,000 amol or 40,000 amol. Hybrid capture was performed in duplicate, and the degree of HPV DNA enrichment in post-capture libraries was determined using HPV-16 E6 and E7 dPCR assays. Results were normalized to the 72 hr at 47° C. condition with 4,000 amol per bait.

FIG. 7: Analytical sensitivity of HPV-seq. Results from the dilution series presented in FIG. 2b are displayed as genome equivalents (GE) of HPV DNA. Expected GE (x-axis) and observed GE from HPV-seq (y-axis) were highly correlated. The LLOD was 0.2 GE.

FIG. 8: IGV snapshots for the 4 dPRC negative but HPV-seq positive end-of-treatment samples. The dual-strand full-length HPV-16 hybrid capture panel was used. Following processing of sequenced reads through ConsensusCruncher, unique (all.unique.dcs) reads were displayed as mapped to the HPV-16 genome.

### DETAILED DESCRIPTION

In the following description, numerous specific details are set forth to provide a thorough understanding of the invention. However, it is understood that the invention may be practiced without these specific details.

Robust detection of circulating tumor (ct)DNA fragments can play a role in cancer screening and measurement of minimal residual disease but has remained a challenge in these low-disease-burden settings. For human papillomavirus (HPV)-associated malignancies, HPV DNA offers a convenient and relevant ctDNA marker. Digital PCR (dPCR) is the most common approach for HPV ctDNA detection, but the reported sensitivity and specificity of this current technology is likely insufficient for clinical applications in patients with low disease burden.

We developed a new next-generation sequencing approach (HPV-seq) for quantitative and qualitative assessment of HPV ctDNA fragments. Our approach involves enhanced recovery of viral sequences from plasma cell-free DNA by hybrid capture. Specifically, we performed hybridization to both sense and antisense (dual-strand) biotinylated DNA baits tiled across the full-length double-stranded DNA HPV genome. We evaluated the performance characteristics of HPV-seq using cancer cell lines and plasma cell-free DNA samples from a cohort of 8 locally-advanced cervix cancer patients (NCT02388698). HPV-seq results were compared with dPCR results from this cohort.

Dual-strand hybrid capture recovered 67-98% more HPV fragments than standard hybrid capture. This enabled reproducible detection using HPV-seq of <0.01% of HPV ctDNA in a cell line dilution series. In cervix cancer patients, HPV-seq and dPCR results were highly correlated (R=0.99, p=2.8×10⁻¹²) with HPV-seq detecting ctDNA at levels down to 0.004% (30-fold lower than dPCR) in dPCR-negative post-treatment samples. Accurate HPV genotyping was successful from 2/2 pre-treatment plasma samples, and HPV ctDNA was detected in 3/3 end-of-treatment plasma samples from patients with subsequent recurrence. HPV ctDNA fragment sizes were consistently shorter than non-cancer-derived cell-free DNA fragments (median 146 vs 169 bp, p<10⁻³²⁰) regardless of HPV genotype (HPV-16 vs HPV-33) or clinical setting (recurrence vs non-recurrence).

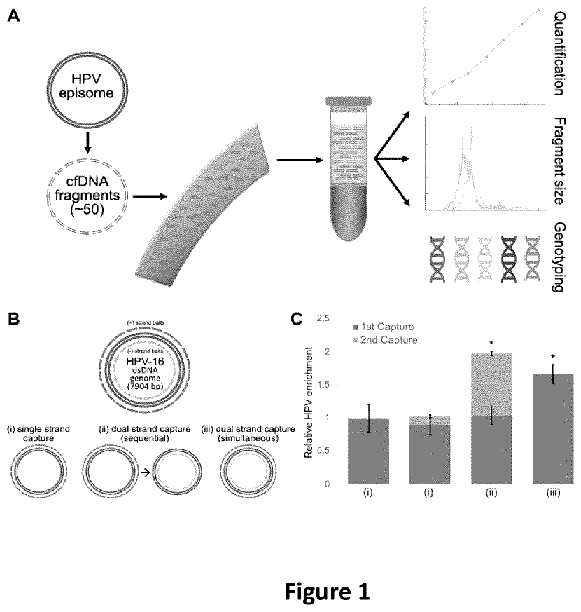

HPV-seq is a quantitative and sensitive method for ctDNA detection. HPV-seq also reveals qualitative information about ctDNA fragments such as HPV genotype and ctDNA fragment length distribution, which can be utilized to improve the specificity of detection (FIG. 1a). Our findings will have implications for treatment monitoring of disease burden in HPV-related cancers and could open the door to new potential clinical applications of HPV ctDNA analysis.

In an aspect, there is provided a method for capturing circulating tumor DNA (ctDNA) of interest from an animal sample, preferably a mammalian sample, further preferably a human patient sample, comprising cell-free DNA (cfDNA), the method comprising: adding to the patient sample a library of nucleic acid hybrid capture probes, wherein the library of probes is complementary to both strands of the double stranded ctDNA of interest and the probes are tagged for capture; allowing the probes to hybridize to the ctDNA; and capturing the hybridized ctDNA using the tag on the probes.

In some embodiments, the method further comprises polymerase chain reaction (PCR) assembly to detect a specific ctDNA of interest.

In some embodiments, the method further comprises sequencing the captured ctDNA. Preferably, the sequencing comprises next-generation sequencing. Further preferably, the next-generation sequencing comprises Illumina, Roche 454, or Ion Torrent Sequencing.

In some embodiments, the probes complementary to one strand of the ctDNA are offset by 40-60% from the probes complementary to other strand. Preferably, the probes complementary to one strand of the ctDNA are offset by 50% from the probes complementary to other strand.

In some embodiments, the probes are 50 bp to 160 bp in length, between 80 bp to 160 bp in length, between 100 bp and 140 bp in length, or about 120 bp in length.

In some embodiments, the ctDNA of interest corresponds to a mutation of interest.

In some embodiments, the ctDNA of interest corresponds to a virus, preferably an oncogenic virus. Preferably, the oncogenic virus is selected from the group consisting of human papillomavirus (HPV), Epstein-Barr virus (EBV), Kaposi's sarcoma-associated herpesvirus (KSHV), human T-lymphotropic virus (HTLV), or Merkle cell polyomavirus (MCV). In some embodiments, the oncogenic virus is human papillomavirus (HPV).

In some embodiments, the library of probes covers substantially the entire genome of the oncogenic virus.

In some embodiments, the method further comprises at least one of determining the fragment length of the ctDNA, genotyping the ctDNA, and mapping location of the ctDNA to the genome.

In some embodiments, the patient sample is selected from the group consisting of peripheral blood serum or plasma, urine, saliva, breast milk, cerebrospinal fluid, and synovial fluid. In a specific embodiment, the patient sample is peripheral blood plasma.

In some embodiments, the methods described herein are for detecting cancer.

In some embodiments, the methods described herein are for monitoring cancer therapy.

In an aspect, there is provided a library of probes complementary to both strands of a double stranded ctDNA of interest and the probes are tagged for capture.

In some embodiments, the probes complementary to one strand of the ctDNA are offset by 40-60% from the probes complementary to other strand. Preferably, the probes complementary to one strand of the ctDNA are offset by 50% from the probes complementary to other strand.

In some embodiments, the probes in the library are 50 bp to 160 bp in length, between 80 bp to 160 bp in length, between 100 bp and 140 bp in length, or about 120 bp in length.

In some embodiments, the ctDNA of interest corresponds to a virus, preferably an oncogenic virus. Preferably, the oncogenic virus is selected from the group consisting of human papillomavirus (HPV), Epstein-Barr virus (EBV), Kaposi's sarcoma-associated herpesvirus (KSHV), human T-lymphotropic virus (HTLV), or Merkle cell polyomavirus (MCV). In some embodiments, the oncogenic virus is human papillomavirus (HPV).

In some embodiments, the library of probes covers substantially the entire genome of the oncogenic virus.

The advantages of the present invention are further illustrated by the following examples. The examples and their particular details set forth herein are presented for illustration only and should not be construed as a limitation on the claims of the present invention.

### EXAMPLES

### Methods and Materials

### Cell Lines

The SiHa cervix cancer cell line, which harbors a single integrated copy of HPV-16, was obtained from ATCC (Cat #HTB-35). The FaDu head and neck squamous cell carcinoma cell line, which is HPV-negative, was a kind gift of Dr. Bradly Wouters (Princess Margaret Cancer Center). The identity of all cell lines was confirmed via STR profiling, and mycoplasma testing was performed prior to their utilization. Genomic DNA was purified using DNeasy kits (Qiagen) and quantified by Qubit (Life Technologies).

### Patients and Samples

This study was approved by the Ontario Cancer Research Ethics Board. Patients with locally advanced cervix cancer treated with standard-of-care chemoradiotherapy were accrued onto a prospective clinical trial (NCT02388698) for correlative biomarker analysis. Results of the primary analysis were previously reported¹⁴. Patients who consented to optional biobanking and with sufficient stored biospecimens were included in this study. Progression-free survival was measured from the date of diagnosis. The final cohort included 8 HPV-positive cervix cancer patients. Healthy donor plasma samples were obtained following the University Health Network's Research Ethics Board approval. Plasma processing, DNA extraction, and quality assessment were performed as previously described¹⁴.

### Targeted Panel Design

For hybrid capture, panels of 5′-biotinylated 120 nt single-stranded DNA baits (xGen Lockdown probes) were designed and synthesized by IDT (Coralville, Iowa, USA). Each panel consisted of baits targeting HPV sequences as well as regions of 12 human genes frequently mutated in squamous cell carcinomas (CASP8, CDKN2A, EP300, FBXW7, HRAS, MAPK1, NFE2L2, NOTCH1, PIK3CA, PTEN, TERT, TP53). Six different HPV panels were used in this study: (i) HPV genotyping panel consisting of (+) strand baits targeting the E6 and E7 genes from each of 38 HPV types; (ii) HPV-16 (+) strand baits targeting the full-length viral genome; (iii) HPV-16 (−) strand baits targeting the full-length viral genome; (iv) HPV-33 (+) strand baits targeting the full-length viral genome. Baits were 1×tiled (end-to-end) for all full-length viral genome panels, and the HPV-16 (−) strand baits were 50% (60 nt) offset from the HPV-16 (+) strand baits.

### Library Preparation

Illumina-compatible sequencing libraries were prepared from 20 ng of sheared cell line genomic DNA or cfDNA. Cell line genomic DNA was sheared using a Covaris® M220 sonicator (Covaris, Woburn, Mass., USA) followed by cleanup and size selection using Agencourt AMPure XP beads (Beckman Coulter, Brea, Calif., USA). DNA concentration was assessed by Qubit (Life Technologies, Carlsbad, Calif., USA). Sheared SiHa genomic DNA was spiked into sheared FaDu genomic DNA at 10% for initial methodological development and then as an 8-point dilution series (3%, 1%, 0.3%, 0.1%, 0.03%, 0.01%, 0.003%, and 0%) to test analytical sensitivity. Libraries were constructed using KAPA Hyper Prep kits (#KK8504, Kapa Biosystems, Wilmington, Mass., USA) with custom unique molecular identifier (UMI)-containing adapters. Following end repair and A-tailing, adapter ligation was performed overnight using 100-fold molar excess of adapters. Agencourt AMPure XP beads were used for library clean up and ligated fragments were amplified between 4-8 cycles using 0.5 μM IIluminal universal and sample specific index primers. Final library quality control was performed using a Bioanalyzer 2100 (Agilent, Santa Clara, Calif., USA), and concentration was measured using Qubit.

### Hybrid Capture

Hybrid capture was conducted on ˜500 ng of amplified library without multiplexing. For initial methodological development, hybrid capture reactions were carried out with technical duplicates for each condition. Using the 10% SiHa DNA mixture, we evaluated the effect of individual probe concentration and hybridization temperature/duration on target sequence enrichment. Probes were included at either 4000 or 40,000 amol per probe. Hybridization was carried out at either 47° C. for 72 hr or at 65° C. for 18 hr. Thereafter, all other hybrid capture reactions were carried out with 4000 amol per probe at 65° C. for 18 hr. Hybrid capture reactions contained 5 μL of 1 mg/mL Cot-1 DNA and 1 nmol each of the IDT xGen Universal Blocking Oligos (TS-p5 and TS-p7, 8nt). The mixture was dried using a SpeedVac and then re-suspend in 1.1 μL water, 8.5 μL NimbleGen 2× hybridization buffer and 3.4 μL NimbleGen hybridization component A. The mixture was heat denatured at 95° C. for 10 min before adding 4 μL of IDT xGen Lockdown Probes (1000 amol/probe/μL). After 18 hr hybridization at 65° C., target of interests were captured by incubation with 5′-biotinylated oligos, and then were pulled down by Streptavidin M-270 Dynabeads (Thermo Fisher Scientific, Waltham, Mass., USA), followed by buffer wash steps to remove unbound DNA. Captured DNA was subjected to on-beads PCR amplification with 25 μL of 2× KAPA HiFi HotStart ReadyMix, 5 μL of 10 μM Illumina Primermix, and 20 μL of beads captured DNA. Amplified captured DNA was cleaned up with 1× Agencourt AMPure XP beads. Enriched libraries were eluted with TE buffer (IDTE pH 8.0) for downstream analysis. For sequential hybrid capture reactions, following initial hybridization and Streptavidin M-270 Dynabeads incubation, DNA bound to the Dynabeads was separated from the supernatant on a magnetic rack. The supernatant containing the unbound DNA was quickly transferred into another PCR tube for a second round of probe hybridization. Washing of the Dynabeads, on-beads PCR amplification, and DNA clean up was conducted as described above. Enriched libraries from sequential hybrid capture reactions were eluted with IDTE pH 8.0 into individual tubes for downstream analysis.

### Target Sequence Enrichment Analysis

In order to determine the degree of target sequence enrichment in post-capture versus pre-capture libraries, we designed E6 and E7 primers/probe sets for dPCR. For HPV-16 E6: HPV16_E6_Forward, 5′-ACTGTGTCCTGAAGAAAAGCA; HPV16_E6_Reverse, 5′-GTCCACCGACCCCTTATATT; and a double quenched probe 5′-/56-FAM/ACATCTGGA/ZEN/CAAAAAGCAAAGATTCCA/3IABkFQ/. For HPV-16 E7: HPV16_E7_Forward, 5′-GAGGAGGATGAAATAGATGGTC; HPV16_E7_Reverse, 5′-CCGAAGCGTAGAGTCACA; and a double quenched probe 5′-/5HEX/TGGACAAGC/ZEN/AGAACCGGACA/31ABkFQ/. First, pre- and post-capture libraries were quantified using the dPCR Library Quantification Kit for Illumina Truseq (Bio-Rad, Cat. #1863040). All dPCR reactions were carried out using a QX200 Droplet Digital PCR system (Bio-Rad Laboratories, Hercules, Calif., USA). Thermocycling for 40 cycles was performed on a C1000 Touch Thermal Cycler with 96-Deep Well Reaction Module (Bio-Rad). Droplet analysis and copy number quantification was performed using QuantaSoft software (Bio-Rad). We prepared dilutions of 2 ng/μL for pre-capture libraries and 0.02 pg/μL for post-capture libraries; 5 μL of each diluted library was used for E6 and E7 absolute quantification by dPCR. The degree of enrichment of the E6 and E7 sequences was determined by dividing the concentration in post-capture libraries by the concentration in the pre-captured libraries.

### Sequencing Analysis

High throughput DNA sequencing was performed on Illumina NextSeq500 or MiSeq platforms with paired-end reads of ≥75 bp. A 2 nt UMI and a 1 nt invariant spacer sequence were removed from each read³⁶. A thymine base was encoded in the third position for adapter ligation and a spacer filter was enforced to remove reads not compliant with this design. For the remaining reads (>98%), the UM Is were appended into the header for unique molecular identification. Next, reads were aligned to the human reference genome hg19 using BWA-mem (v 0.7.15)³⁷ and SAMtools (v 1.3.1)³⁸, and recalibrated for base quality score using the Genome Analysis ToolKit (GATK) BaseRecalibrator (v 3.4-46) according to the best practice. The aligned reads were input to ConsensusCruncher (https://github.com/pughlab/ConsensusCruncher) to identify unique molecules. ConsensusCruncher is a Python-based tool that amalgamates reads derived from the same DNA template labelled with the same UMI into a consensus read³⁶.

### HPV Genotyping

Human-unmapped reads were extracted and aligned to a database of 38 HPV reference genomes (Table 5). The aligned reads were input to ConsensusCruncher to identify unique molecules. From these unique molecules, properly-paired reads with high mapping quality 30) were considered for genotyping. High mapping quality score was included in our pipeline to improve resolution of genotyping by filtering reads mapping to multiple genomes. Following these steps, the HPV genotype was assigned according to the virus with the most reads. Mutations in HPV sequences were evaluated in Integrated Genomics Viewer⁴⁰.

### HPV Quantification

HPV quantification from full-length viral genome HPV-seq was based on the number of HPV-mapping reads or the HPV genome equivalents (GEs). The HPV GE was calculated by the formula, N (GE)=I (ng)×X %×10³/3.3, where N is the number of GE, I is the input DNA mass (e.g., 20 ng), X is the proportion of HPV-containing genomes in the input DNA. The estimation of X is based on the sequenced unique molecules of HPV and human DNAs. First, the human-unmapped reads were aligned to the HPV genotype-specific reference genome (e.g. HPV-16). Then, aligned reads were input to ConsensusCruncher to identify unique molecules. From the all unique molecules output of ConsensusCruncher, the number of HPV-mapping and (on-target) human-mapping properly-paired reads were determined. Here, no mapping quality control was applied to the HPV-mapping reads in order to maximize the recovery of all HPV-mapping reads (i.e., even reads that might map to multiple HPV genotypes). The proportion of HPV DNAs in the input DNA (i.e., the union of human and HPV DNAs) was estimated by comparing GATK DepthOfCoverage output from HPV-mapping and (on-target) human-mapping properly paired reads.

### Fragment Length Analysis

Lengths of sequenced fragments were evaluated using CollectlnsertSizeMetrics of the Picard tool (http://broadinstitute.github.io/picard/). In this study, the input bam files were ‘all unique molecules’ generated by ConsensusCruncher. Human-mapping and HPV-mapping reads were analyzed separately for comparison.

### Small Nucleotide Variant Calling

Mutation data from the SiHa and FaDu cell lines were obtained from the COSMIC database⁴¹. Within the 9 kb human gene capture panel that was part of HPV-seq, SiHa harbors one unique mutation within the CASP8 gene (chr2:202131411; c.202C>T). In the cell line dilution series, variants were called by intersecting output from iDES⁷ and Vardict 2⁴² with requirement of supporting reads.

### Software and Statistical Analysis

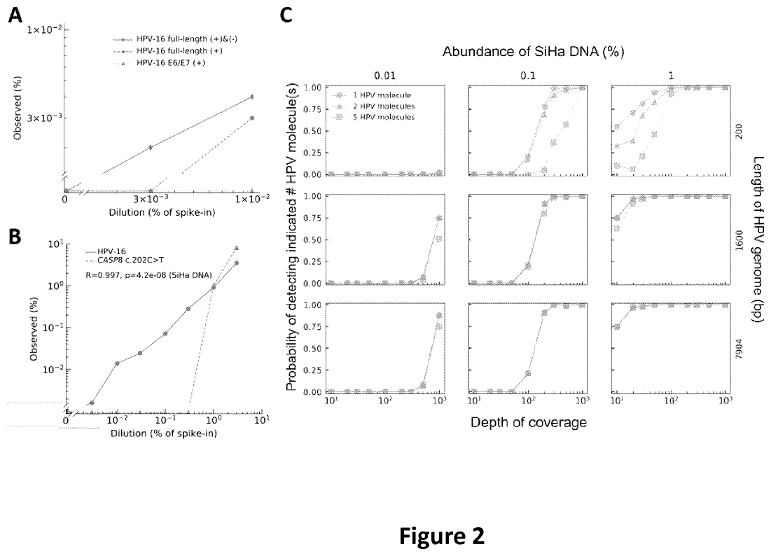

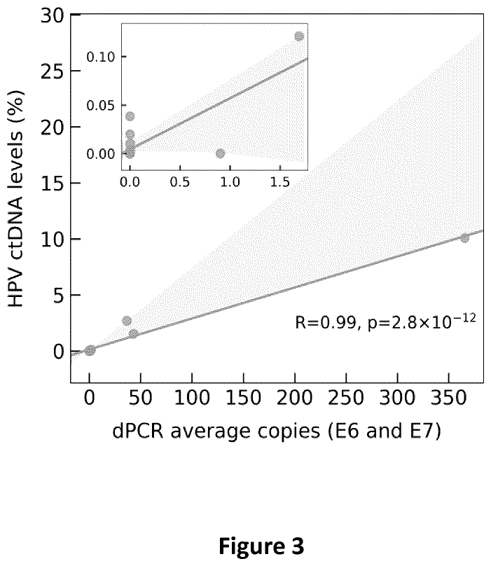

Pearson correlation coefficients and p-values were calculated for dPCR and HPV-seq results using SciPy⁴³ in Python v3.7.2⁴⁴. The Student's t-test was used to test statistical significance of the degree of HPV sequence enrichment as well as differences in fragment lengths between human and HPV DNA fragments using R v3.5.1 (http://www.R-project.org/). Error bars and ranges indicate standard deviation. The probability of HPV detection in FIG. 2 was estimated based on 100-times downsamplings on each coverage level. For FIG. 3, the linear regression with associated 95% confidence interval was generated using seaborn (https://doi.org/10.5281/zenodo.12710). For FIG. 2, data was plotted using brokenaxes (https://qithub.com/bendichter/brokenaxes) and matplotlib v2.2.3 (https://doi.org/10.5281/zenodo.1343133). The Mann Whitney test was used to test differences in end-of-treatment HPV ctDNA levels between recurrent and non-recurrent patients. Kaplan-Meier analysis and log-rank test were performed in SAS v9.3 (SAS Institute, Cary, N.C., USA). All tests were two-tailed.

### Results and Discussion

Enhanced Enrichment of Viral DNA from Sequencing Libraries

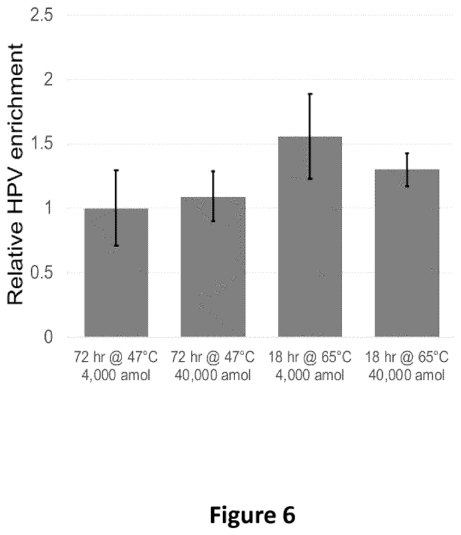

Efficient recovery of target sequences from cfDNA is necessary for robust ctDNA detection by hybrid capture sequencing. We first evaluated the degree of enrichment of HPV sequences using a range of hybrid capture conditions. Fragmented cell line genomic DNA containing 10% SiHa (cervix cancer cell line with single integrated full-length HPV-16 genome) was used to simulate cfDNA. Following construction of Illumina-compatible sequencing libraries, single-stranded sense (+) biotinylated DNA baits were used at a range of concentrations for hybrid capture. Increasing concentrations up to 4000 amol per bait produced greater enrichment (data not shown), but concentrations above 4000 amol per bait did not improve target recovery as measured by dPCR (FIG. 6). Moreover, at 4000 amol per bait, there was no significant impact on target recovery from increasing hybrid capture incubation time from 18 hr at 65° C. to 72 hr at 47° C. Thus, 4000 amol per bait with 18 hr incubation at 65° C. provided optimal experimental conditions that were utilized for all subsequent analyses.

Next, we asked whether the hybrid capture reaction had harvested all of the target sequences in the library. To test this, we examined the unbound library following hybrid capture to see whether there were retained HPV sequences. Subjecting the unbound library to another round of hybrid capture using the same single-stranded (+) baits had a minor impact on total target recovery (FIG. 1b-c), suggesting that the hybrid capture reaction had approached saturation as expected. However, substituting the (+) baits for staggered antisense (−) baits (see Methods) in the second round of hybrid capture resulted in a dramatic improvement in the degree of enrichment of target sequences (98±16% increase; p=3.1×10⁻⁴). This effect was maintained when the (+) and (−) baits were all combined in a single capture reaction (67±14% increase; p=1.8×10⁻³). Based on these results, dual-strand hybrid capture may improve upon standard target enrichment procedures for robust ctDNA detection.

### HPV-Seq Enables Ultrasensitive Detection of Cancer DNA

Having established our hybrid capture methodology for HPV-seq, we next sought to evaluate its analytical sensitivity and lower limit of detection (LLOD). Reported qPCR/dPCR approaches for HPV ctDNA detection have LLOD of ≥GE=¹⁷, which may not be sufficient for low-disease-burden settings, so we tested the ability of HPV-seq to detect HPV DNA at levels <1 GE. HPV-seq with dual-strand full-length viral capture was able to detect SiHa DNA at 0.01% and 0.003%, corresponding to 0.6 and 0.2 GE, respectively, with no signal in the negative control (0%) condition (FIG. 2a), indicating robust LLOD at <1 GE. Of note, 16 unique HPV DNA fragments were recovered at the 0.003% dilution level, indicating that even lower levels might still be detectable. In contrast, single-strand full-length viral capture failed to detect SiHA DNA at 0.003%, and single-strand partial-length (360 bps within the E6 and E7 genes) viral capture did not detect SiHa DNA at either level.

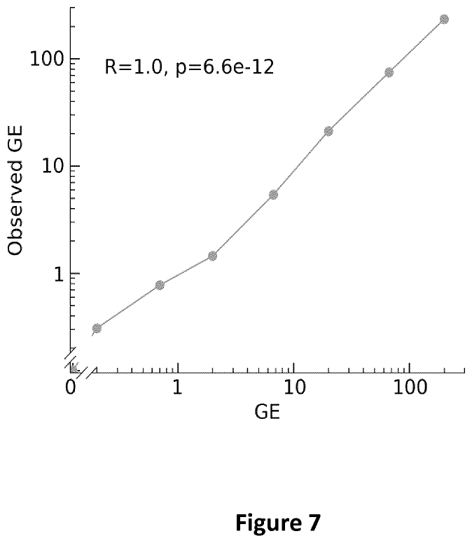

We next compared the analytical sensitivity of HPV-seq to that of mutation detection following hybrid capture. The baits used for HPV-seq encompassed a single clonal heterozygous mutation in SiHa within the CASP8 gene, allowing us to evaluate LLOD in a dilution series. The LLOD using just the CASP8 mutation was only 1%, whereas HPV detection again provided a LLOD 0.003% and displayed a high correlation with the expected levels (R=0.997, p=4.2×10⁻⁰⁸; FIG. 2b and FIG. 7). These results highlight the potential of utilizing HPV DNA fragments to drive analytical sensitivity.

When fragmented to the typical size of cfDNA (˜140-180 bp), the 7.9 kb HPV-16 genome should yield ˜50 independent cancer-specific markers. We reasoned that the LLOD in the SiHa dilution series would be dependent on this large number of independent markers of HPV-16. Indeed, the smaller E6/E7 capture baits failed to produce robust detection of SiHa DNA at <1 GE (FIG. 2a). To further test this hypothesis, we subsampled the sequencing reads from HPV-seq with dual-strand full-length viral capture conducted on 0.01%, 0.1%, and 1% fragmented SiHa DNA (FIG. 2c). Reads were subsampled at various depths of coverage ranging from 10- to 1000-fold. We evaluated reads mapping to 200 bp, 1600 bp, or the full-length 7904 bp HPV-16 genome to simulate the effect of variable numbers of independent markers on LLOD. As expected, the bait lengths for the HPV-16 genome increased the likelihood of HPV detection, with improved analytical sensitivity observed across the three dilution levels. Taken together, these results show the impact of dual-strand full-length viral capture on ultrasensitive detection of HPV.

HPV-Seq for ctDNA Detection and Genotyping in Cervix Cancer Patients

Uterine cervix cancer is one of several cancer types that is commonly HPV-associated. We previously evaluated the clinical utility of HPV genotype-matched dPCR from plasma cfDNA for treatment response monitoring in a cohort of locally advanced cervix cancer patients¹⁴. In this prospective multi-center series, baseline (pre-treatment) sensitivity was high, but the small dynamic range in longitudinal samples presented challenges for interpreting results at end-of-treatment or post-treatment. We therefore asked whether HPV-seq could improve upon the quantitative and qualitative information obtained from these plasma cfDNA samples. Of the 57 samples from the previous study, 18 samples were subjected to HPV-seq (Table 2) using either (i) a HPV genotyping panel (single-strand baits targeting the E6 and E7 genes from each of 38 HPV types; N=2), (ii) dual-strand full-length viral capture for HPV-16 (N=14), or (iii) single-strand full-length viral capture for other HPV types (N=2). The HPV genotyping panel (i) allowed us to test whether HPV-seq could provide HPV genotype information directly from plasma, while the full-length viral capture panels (ii, iii) were used to test HPV-seq as a monitoring tool for detecting residual disease. Considering all samples, HPV ctDNA levels detected by HPV-seq were correlated with dPCR results (R=0.99, p=7.9×10⁻¹⁴).

We evaluated the performance of HPV-seq for HPV genotyping from baseline plasma samples by comparing to cervical swab genotyping results previously reported for this cohort¹⁴. Both analysed patients harbored tumors positive for HPV-16. HPV ctDNA was detected in both samples but not in a healthy control sample. For each patient, between 80-100% of mapping reads were assigned to the same HPV genotype that was observed in the tumor tissue (Table 1). Considering the HPV genotype with the largest number of mapped reads, HPV-seq provided an overall accuracy of 100% for genotyping in this cohort. Of note, genotyping was possible even for samples with relatively low ctDNA levels (<10 copies by dPCR). These results suggest that HPV-seq using a broad genotyping panel could replace tumor tissue analysis for accurate HPV genotyping.

TABLE 1
HPV genotyping using HPV-seq

| | Tissue | | | HPV molecules from plasma HPV-seq^{#} | | | | | | Plasma | HPV-seq |
|---|---|---|---|---|---|---|---|---|---|---|---|
| | HPV | dPCR copy # | | HPV | HPV | HPV | HPV | HPV | HPV | type* | accuracy |
| Patient | type | E6 | E7 | 16 | 18 | 31 | 33 | 35 | 45 | HPV | genotype |
| P2 | HPV-16 | 1.54 | 1.54 | 8 | 2 | 0 | 0 | 0 | 0 | HPV-16 | 80% |
| P6 | HPV-16 | 6.16 | 22 | 84 | 0 | 0 | 0 | 0 | 0 | HPV-16 | 100% |
| Control | — | 0 | 0 | 0 | 0 | 0 | 0 | 0 | 0 | — | 100% |

^{#}The number of properly-paired reads with high mapping quality score (≥30) on the HPV genome.
*Plasma HPV genotype based on the viral genome with highest number of properly-paired mapped reads.

HPV ctDNA Quantification in Cervix Cancer Patients

With the success of HPV-seq for genotyping from baseline samples, we next asked whether HPV-seq using full-length virus capture would enable treatment response monitoring. We subjected a total of 16 patient samples and 4 healthy control samples to HPV-seq using full-length virus capture. All patient samples were obtained either at end-of-treatment, 3 months post-treatment, or at the time of recurrence. HPV ctDNA levels according to HPV-seq and dPCR were highly correlated (R=0.99, p=2.8×10⁻¹²; FIG. 3). Three samples contained <1 GE of HPV with the lowest detected level by HPV-seq and dPCR at 0.24 GE (0.004%) and 1.69 (0.12%), respectively (Table 3). None of the 4 healthy control samples had detectable HPV-seq signal.

Of the 5 patient samples with dPCR-positive results (2 at end-of-treatment, 3 at post-treatment/recurrence), HPV-seq produced positive results in 4 (80%) samples (Table 3). The 1 sample with dPCR-positive HPV-seq-negative results had low dPCR values (˜1 copy) and was from a patient who remained disease-free with 43 months of follow up.

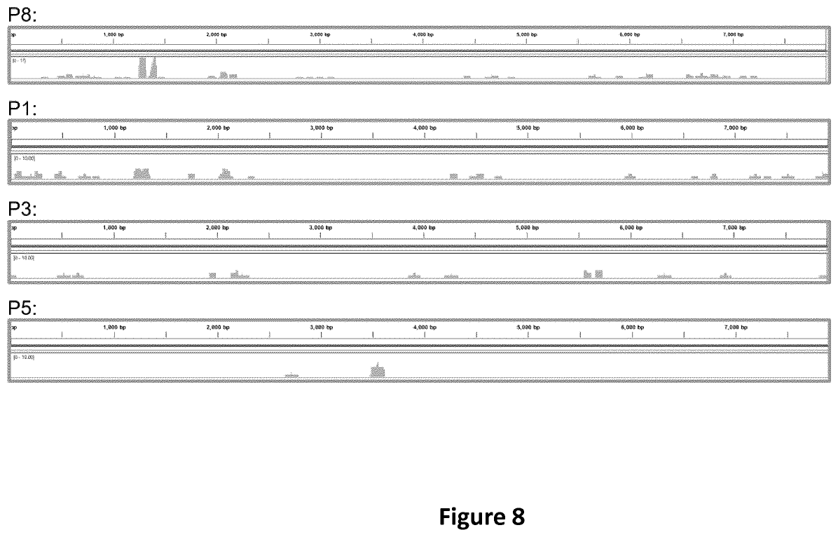

Of the 11 patient samples with dPCR-negative results, 7 (64%) were also negative by HPV-seq (Table 3). The 4 dPCR-negative HPV-seq-positive results were further analyzed to assess whether they represented false positive HPV-seq results. Of note, each of these 4 samples were at the end-of-treatment timepoint using the dual-strand full-length viral capture baits. Interestingly, one of these samples (from P5) displayed a low level of ctDNA (0.004%, 0.24 GE) 12 months prior to recurrence with a biopsy-proven solitary 1.2-cm lung metastasis, although the 3 months post-treatment sample from this patient was falsely negative by both HPV-seq and dPCR. None of the other 3 patients with dPCR-negative HPV-seq-positive results have experienced disease relapse after >36 months of follow up. Supporting the veracity of the HPV-seq results, the HPV-mapping reads were distributed across the HPV-16 genome (FIG. 8) and displayed high mapping quality scores (Table 3).

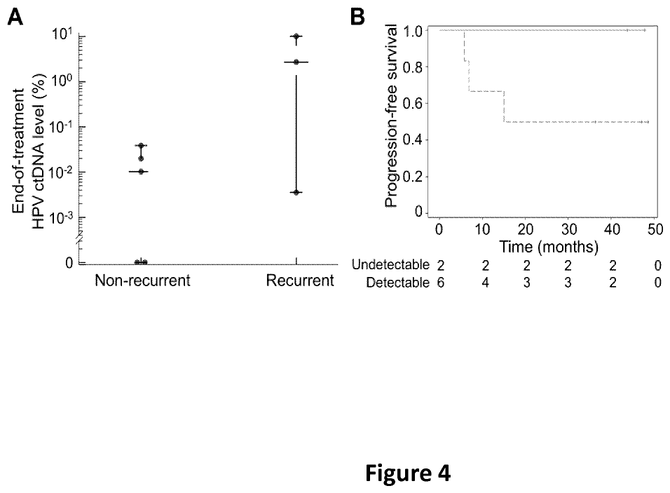

Three (P4, P5, and P7) of the 8 patients included in this study experienced disease recurrence. HPV ctDNA levels were on average higher at the end-of-treatment timepoint among those 3 patients compared with the 5 patients who did not relapse (p=0.23 FIG. 4a). With a median follow up of 27.5 months (range 4.8-48.6), detectable HPV ctDNA at the end-of-treatment timepoint was associated with poor progression-free survival (log-rank p=0.26; FIG. 4b). Sensitivity HPV-seq for predicting recurrence at the end-of-treatment timepoint was 100%.

ctDNA Fragment Lengths in Cervix Cancer Patients

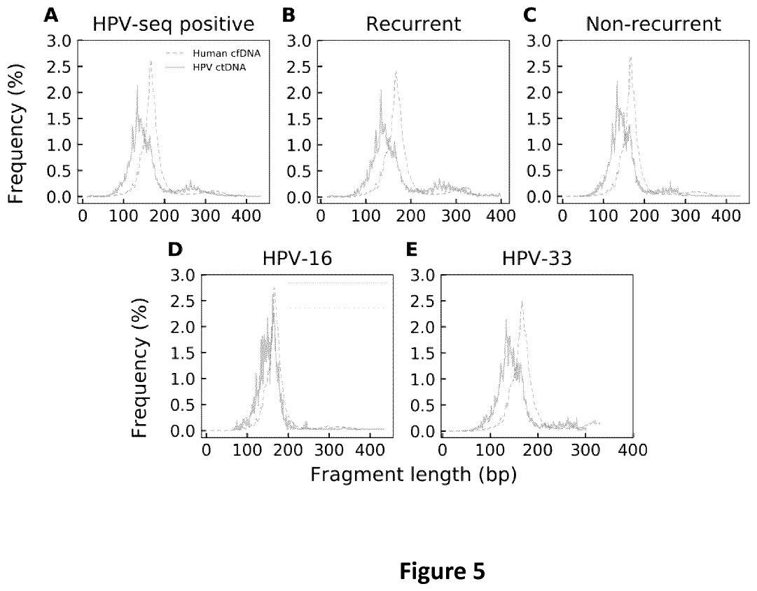

Fragment lengths of sequenced cfDNA fragments may be useful for discriminating between healthy and malignant tissues-of-origin^{6,24-28}. Shorter fragments have been observed in ctDNA from a variety of cancer types, including nasopharyngeal carcinoma and hepatocellular carcinoma, but this has not been previously evaluated in HPV-associated cancers. We compared HPV-mapping ctDNA fragment lengths to human-mapping cfDNA fragment from samples analyzed with full-length viral capture HPV-seq. HPV ctDNA displayed a median fragment size of 146 bp, which was 23 bp shorter than the median fragment size of human-mapping cfDNA (FIG. 5a and Table 4). Of note, similar results were seen among samples taken at the time of recurrence (FIG. 5b) as well as samples taken from patients who have not recurred (FIG. 5c). Regardless of HPV genotype, median HPV ctDNA fragment length was shorter than human-mapping cfDNA (FIG. 5d-e). Taken together, along with HPV genotyping, these results illustrate the capability to perform qualitative analyses of ctDNA fragments using HPV-seq.

### DISCUSSION

High analytical sensitivity is a prerequisite for a ctDNA test applied to oncology settings with low disease burden¹. Here, we validated the concept that simultaneous assessment of multiple cancer-specific markers in a given patient has a major impact on analytical performance of ctDNA testing. Specifically, we used HPV-associated cervix cancer as a highly relevant model, showing for the first time that HPV-seq using dual-strand full-length viral capture enables robust detection of <1 GE of ctDNA. We also showed that HPV-seq reveals qualitative information regarding ctDNA fragments such as HPV genotype, mapping location along the HPV genome, and ctDNA fragment length distribution that cannot be easily inferred from qPCR/dPCR.

Virus-associated cancers have long served as a model for ctDNA methodology development and clinical utility testing. Epstein-Barr virus (EBV) provides a convenient marker for nasopharyngeal and other cancers, but sensitivity and specificity shortfalls of the established qPCR test present barriers to its use for early detection and response monitoring³⁰⁻³². Recently, full-length viral capture sequencing was reported to improve the specificity of EBV detection in a prospective screening study. Ours is the first study to show the potential for this approach to also drive greater sensitivity in virus-associated cancers.

Despite its limitations, the plasma EBV DNA qPCR test continues to be employed clinically and in trials. The sensitivity of this test is aided by targeting of a conserved amplicon in the EBV genome. The HPV genome, however, does not harbor any repeated sequences that lend themselves to more sensitive qPCR/dPCR assays. Moreover, distinct high-risk oncogenic HPV genotypes have widely divergent sequences. These features make HPV detection ideal for hybrid capture sequencing methods due to the flexibility for incorporating hundreds of baits spanning the entire genome of multiple viral genotypes.

We found that dual-strand capture was a key element for enabling robust detection of <1 GE of ctDNA. While single-strand and partial-genome viral capture were also able to produce quantitative and qualitative HPV-seq results in our study, the most robust ultrasensitive ctDNA detection was achieved using dual-strand full-length viral capture. Sequential capture of the complementary strands was feasible but laborious, so we instead implemented an approach for simultaneous dual-strand capture using staggered reverse-complementary baits. This approach is applicable to any hybrid capture sequencing workflow and, importantly, could therefore have an immediate impact for ctDNA applications in other cancer types as well.

Locally advanced cervix cancer is managed with concurrent chemoradiotherapy. The role of subsequent adjuvant therapy is currently being evaluated in multiple international clinical trials (e.g., NCT00980954, NCT01414608, NCT02036164, NCT0270396, NCT02853604, and NCT03468010). We previously reported on the capability of HPV genotype-specific dPCR from end-of-treatment plasma cfDNA to pre-date clinical recurrence in locally advanced cervix cancer patients. From that study, we found low levels of HPV ctDNA using dPCR in the end-of-treatment timepoint from 3 patients who have remained disease-free. Using HPV-seq, 2 of these 3 samples had no detectable HPV fragments, suggesting that they may have been false-positive dPCR results. Conversely, 4 (36%) of the dPCR-negative surveillance samples harbored residual HPV ctDNA fragments detectable by HPV-seq. This again occurred specifically in the end-of-treatment samples, including from 3 patients who have remained disease-free. Importantly, there were no qualitative attributes (mapping quality, fragment length) of HPV DNA fragments in these samples that would easily distinguish them from the dPCR-positive samples, suggesting that these represented bona fide ctDNA molecules.

There is no current standard timepoint for minimal residual disease ctDNA testing in chemoradiotherapy-treated patients. Based on our results, at the end-of-treatment timepoint sensitivity was excellent (100%) for minimal residual disease detection using HPV-seq. If this high sensitivity is confirmed in larger studies, patients with undetectable end-of-treatment ctDNA levels using HPV-seq could be candidates for observation and thus spared adjuvant therapy or intensive surveillance. However, a positive end-of-treatment result might not guarantee subsequent recurrence and could necessitate repeat testing or else alternative surveillance procedures (e.g., medical imaging). Future studies should also consider later timepoints (e.g., 4-6 weeks post-treatment) to allow for additional clearance of residual ctDNA from effectively treated tumor cells.

The findings of this study will require validation in larger cohorts to confirm the clinical utility for quantitative and qualitative ctDNA analysis using HPV-seq. Future studies will also need to expand the utility of HPV-seq to other HPV associated cancers including oropharynx cancer and anogenital cancer types. The addition of other orthogonal markers of HPV in the circulation such as antibodies or cfDNA methylation patterns may lead to more refined and robust biomarker panels for measuring disease burden and response to therapy in HPV-associated cancers. Application of our methods to cancer screening settings should also be explored.

In conclusion, HPV-seq enables ultrasensitive detection and qualitative analysis of cervix cancer ctDNA. This study will have implications for treatment monitoring of HPV-related cancers and could open the door to new potential clinical applications of HPV ctDNA analysis, including for early detection and minimal residual disease testing.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

### References

1. Corcoran, R. B. & Chabner, B. A. Application of Cell-free DNA Analysis to Cancer Treatment. N. Engl. J. Med. 379, 1754-1765 (2018).
2. Burgener, J. M., Rostami, A., De Carvalho, D. D. & Bratman, S. V. Cell-Free DNA as a Post-Treatment Surveillance Strategy: Current Status. Semin. Oncol. (2018). doi:10.1053/j.seminonco1.2018.01.009
3. Newman, A. M. et al. An ultrasensitive method for quantitating circulating tumor DNA with broad patient coverage. Nat. Med. 20, 548-554 (2014).
4. Shen, S. Y. et al. Sensitive tumour detection and classification using plasma cell-free DNA methylomes. Nature 563, 579-583 (2018).
5. Abbosh, C. et al. Phylogenetic ctDNA analysis depicts early-stage lung cancer evolution. Nature 545, 446-451 (2017).
6. Cristiano, S. et al. Genome-wide cell-free DNA fragmentation in patients with cancer. Nature 570, 385-389 (2019).
7. Newman, A. M. et al. Integrated digital error suppression for improved detection of circulating tumor DNA. Nat. Biotechnol. 34, 547-555 (2016).
8. Chaudhuri, A. A. et al. Early Detection of Molecular Residual Disease in Localized Lung Cancer by Circulating Tumor DNA Profiling. Cancer Discov. 7, 1394-1403 (2017).
9. McDonald, B. R. et al. Detection of residual disease after neoadjuvant therapy in breast cancer using personalized circulating tumor DNA analysis. bioRxiv 425470 (2018). doi:10.1101/425470
10. Pornthanakasem, W. et al. Human papillomavirus DNA in plasma of patients with cervical cancer. BMC Cancer 1, 2 (2001).
11. Lo, Y. M. et al. Quantitative analysis of cell-free Epstein-Barr virus DNA in plasma of patients with nasopharyngeal carcinoma. Cancer Res. 59, 1188-1191 (1999).
12. Cao, H. et al. Quantitation of human papillomavirus DNA in plasma of oropharyngeal carcinoma patients. Int. J. Radiat. Oncol. Biol. Phys. 82, e351-8 (2012).
13. Kim, K. Y. et al. Clinical Utility of Epstein-Barr Virus DNA Testing in the Treatment of Nasopharyngeal Carcinoma Patients. International Journal of Radiation Oncology*Biology*Physics 98, 996-1001 (2017).
14. Han, K. et al. Circulating Human Papillomavirus DNA as a Biomarker of Response in Patients With Locally Advanced Cervical Cancer Treated With Definitive Chemoradiation. JCO Precision Oncology 2, 1-8 (2018).
15. Higginson, D. S. et al. HPV cell free DNA as a marker of response to chemoradiation for HPV-positive oropharngeal cancers. Journal of Clinical Oncology 34, 6083-6083 (2016).
16. Damerla, R. R. et al. Detection of Early Human Papillomavirus-Associated Cancers by Liquid Biopsy. JCO Precision Oncology 1-17 (2019).
17. Chera, B. S. et al. Rapid Clearance Profile of Plasma Circulating Tumor HPV Type 16 DNA during Chemoradiotherapy Correlates with Disease Control in HPV-Associated Oropharyngeal Cancer. Clin. Cancer Res. (2019). doi:10.1158/1078-0432.CCR-19-0211
18. Bernard-Tessier, A. et al. Clinical Validity of HPV Circulating Tumor DNA in Advanced Anal Carcinoma: An Ancillary Study to the Epitopes-HPV02 Trial. Clin. Cancer Res. 25, 2109-2115 (2019).
19. Cabel, L. et al. Prognostic Impact of Residual HPV ctDNA Detection after Chemoradiotherapy for Anal Squamous Cell Carcinoma. Clin. Cancer Res. 24, 5767-5771 (2018).
20. Hanna, G. J. et al. Plasma HPV cell-free DNA monitoring in advanced HPV-associated oropharyngeal cancer. Ann. Oncol. 29, 1980-1986 (2018).
21. Olmedillas-Lopez, S., Garcia-Arranz, M. & Garcia-Olmo, D. Current and Emerging Applications of Droplet Digital PCR in Oncology. Mol. Diagn. Ther. 21, 493-510 (2017).
22. Kim, K. Y. et al. Current State of PCR-Based Epstein-Barr Virus DNA Testing for Nasopharyngeal Cancer. J. Natl. Cancer Inst. 109, (2017).
23. Le, Q.-T. et al. An international collaboration to harmonize the quantitative plasma Epstein-Barr virus DNA assay for future biomarker-guided trials in nasopharyngeal carcinoma. Clin. Cancer Res. 19, 2208-2215 (2013).
24. Lam, W. K. J. et al. Sequencing-based counting and size profiling of plasma Epstein-Barr virus DNA enhance population screening of nasopharyngeal carcinoma. Proc. Natl. Acad. Sci. U.S.A 115, E5115-E5124 (2018).
25. Jiang, P. et al. Lengthening and shortening of plasma DNA in hepatocellular carcinoma patients. Proc. Natl. Acad. Sci. U.S.A (2015). doi:10.1073/pnas.1500076112
26. Mouliere, F. et al. Enhanced detection of circulating tumor DNA by fragment size analysis. Sci. Transl. Med. 10, (2018).
27. Snyder, M. W., Kircher, M., Hill, A. J., Daza, R. M. & Shendure, J. Cell-free DNA Comprises an In Vivo Nucleosome Footprint that Informs Its Tissues-Of-Origin. Cell 164, 57-68 (2016).
28. Underhill, H. R. et al. Fragment Length of Circulating Tumor DNA. PLoS Genet. 12, e1006162 (2016).
29. Lam, W. K. J., Chan, K. C. A. & Lo, Y. M. D. Plasma Epstein-Barr virus DNA as an archetypal circulating tumour DNA marker. J. Pathol. 247, 641-649 (2019).
30. Chan, K. C. A. et al. Analysis of plasma Epstein-Barr virus DNA to screen for nasopharyngeal cancer. N. Engl. J. Med. 377, 513-522 (2017).
31. Lin, J.-C. et al. Quantification of Plasma Epstein-Barr Virus DNA in Patients with Advanced Nasopharyngeal Carcinoma. N. Engl. J. Med. 350, 2461-2470 (2004).
32. Chan, A. T. C. et al. Plasma Epstein-Barr virus DNA and residual disease after radiotherapy for undifferentiated nasopharyngeal carcinoma. J. Natl. Cancer Inst. 94, 1614-1619 (2002).
33. Le, Q.-T. et al. A comparison study of different PCR assays in measuring circulating plasma epstein-barr virus DNA levels in patients with nasopharyngeal carcinoma. Clin. Cancer Res. 11, 5700-5707 (2005).
34. Bernard, H.-U., Calleja-Macias, I. E. & Dunn, S. T. Genome variation of human papillomavirus types: phylogenetic and medical implications. Int. J. Cancer 118, 1071-1076 (2006).
35. Akagi, K. et al. Genome-wide analysis of HPV integration in human cancers reveals recurrent, focal genomic instability. Genome Res. 24, 185-199 (2014).
36. Wang, T. T. et al. High efficiency error suppression for accurate detection of low-frequency variants. Nucleic Acids Research (2019). doi:10.1093/nar/gkz474
37. Li, H. & Durbin, R. Fast and accurate short read alignment with Burrows-Wheeler transform. Bioinformatics 25, 1754-1760 (2009).
38. Li, H. et al. The Sequence Alignment/Map format and SAMtools. Bioinformatics 25, 2078-2079 (2009).
39. DePristo, M. A. et al. A framework for variation discovery and genotyping using next-generation DNA sequencing data. Nat. Genet. 43, 491-498 (2011).
40. Robinson, J. T. et al. Integrative genomics viewer. Nat. Biotechnol. 29, 24 (2011).
41. Tate, J. G. et al. COSMIC: the Catalogue Of Somatic Mutations In Cancer. Nucleic Acids Res. 47, D941-D947 (2019).
42. Lai, Z. et al. VarDict: a novel and versatile variant caller for next-generation sequencing in cancer research. Nucleic Acids Res. 44, e108 (2016).
43. Jones, E., Oliphant, T., Peterson, P. & Others. SciPy: Open source scientific tools for Python, 2001. (2016).
44. Van Rossum, G. & Drake, F. L., Jr. Python tutorial. (Centrum voor Wiskunde en Informatica Amsterdam, Netherlands, 1995).

TABLE 2
Patients, timepoints, and the sequencing panels used in this study.

| Patient | Pre-treatment | End-of-treatment | 3MFU | Recurrence | 3MFU/RECR | Control |
|---|---|---|---|---|---|---|
| P1 | — | HPV16(+)&(−) | HPV16(+)&(−) | — | — | — |
| P2 | HPV E6&E7(+) | HPV16(+)&(−) | HPV16(+)&(−) | — | — | — |
| P3 | — | HPV16(+)&(−) | HPV16(+)&(−) | — | — | — |
| P5 | — | HPV16(+)&(−) | HPV16(+)&(−) | HPV16(+)&(−) | — | — |
| P6 | HPV E6&E7(+) | HPV16(+)&(−) | HPV16(+)&(−) | — | — | — |
| P7 | — | HPV16(+)&(−) | — | — | — | — |
| P8 | — | HPV16(+)&(−) | HPV16(+)&(−) | — | — | — |
| P4 | — | HPV33(+) | — | — | HPV33(+) | — |
| cfDNA | — | — | — | — | — | all panels |

Abbreviation:
3MFU = 3 month post-treatment follow up;
RECR = recurrence

TABLE 3
ctDNA quantification using full-length viral capture HPV-seq for 34 plasma cfDNA
samples from cervix cancer patients

| | | Tissue | | | Plasma HPV-seq attributes | | | |
|---|---|---|---|---|---|---|---|---|
| | | HPV | dPCR copy # | | | MAPQ > | | |
| Patient | Timepoint | type | E6 | E7 | # HPV | 30 | % HPV | GE |

HPV dPCR Positive Samples

| | | | | | | | | |
|---|---|---|---|---|---|---|---|---|
| P4 | end-of-treatment | HPV-33 | 389.25 | 342 | 33674 | 98.2% | 10.07% | 671.06 |
| P7 | end-of-treatment | HPV-16 | 39.6 | 33 | 11696 | 99.8% | 2.70% | 180.19 |
| P4 | 3MFU/RECR | HPV-33 | 45 | 40.5 | 6342 | 98.4% | 1.52% | 101.46 |
| P5 | RECR | HPV-16 | 0 | 3.375 | 450 | 99.7% | 0.12% | 8.06 |
| P6 | 3MFU | HPV-16 | 1.8 | 0 | 0 | 0 | 0% | 0 |

HPV dPCR Negative Samples

| | | | | | | | | |
|---|---|---|---|---|---|---|---|---|
| P8 | end-of-treatment | HPV-16 | 0 | 0 | 94 | 98.9% | 0.04% | 2.56 |
| P1 | end-of-treatment | HPV-16 | 0 | 0 | 44 | 100% | 0.02% | 1.32 |
| P3 | end-of-treatment | HPV-16 | 0 | 0 | 24 | 100% | 0.01% | 0.68 |
| P5 | end-of-treatment | HPV-16 | 0 | 0 | 10 | 100% | 0.004% | 0.24 |
| P1 | 3MFU | HPV-16 | 0 | 0 | 0 | — | 0% | 0 |
| P2 | end-of-treatment | HPV-16 | 0 | 0 | 0 | — | 0% | 0 |
| P2 | 3MFU | HPV-16 | 0 | 0 | 0 | — | 0% | 0 |
| P3 | 3MFU | HPV-16 | 0 | 0 | 0 | — | 0% | 0 |
| P5 | 3MFU | HPV-16 | 0 | 0 | 0 | — | 0% | 0 |
| P6 | end-of-treatment | HPV-16 | 0 | 0 | 0 | — | 0% | 0 |
| P8 | 3MFU | HPV-16 | 0 | 0 | 0 | — | 0% | 0 |

Abbreviation:
3MFU = 3 month post-treatment follow up;
RECR = recurrence

TABLE 4
Fragment length distributions for sequenced human and HPV DNAs

| | | HPV | # Fragments | | Median | | Δ fragment | |
|---|---|---|---|---|---|---|---|---|
| Patient | Timepoint | type | Human | HPV | Human | HPV | length | p-value |

Individual Patient Samples
HPV dPCR Positive

| | | | | | | | | |
|---|---|---|---|---|---|---|---|---|
| P4 | end-of-treatment | HPV-33 | 138270 | 10035 | 166 | 143 | 23 | 5.7 × 10⁻³²¹ |
| P7 | end-of-treatment | HPV-16 | 197669 | 3710 | 166 | 153 | 13 | 1.4 × 10⁻²¹⁸ |
| P4 | 3MFU/RECR | HPV-33 | 197744 | 1969 | 171 | 150 | 21 | 5.9 × 10⁻¹²⁵ |
| P5 | RECR | HPV-16 | 153455 | 125 | 167 | 150 | 17 | 3.1 × 10⁻⁰⁷ |

HPV dPCR Negative Samples

| | | | | | | | | |
|---|---|---|---|---|---|---|---|---|
| P8 | end-of-treatment | HPV-16 | 122680 | 33 | 168 | 180 | −12 | 0.01 |
| P1 | end-of-treatment | HPV-16 | 110289 | 13 | 171 | 155 | 16 | 0.2 |
| P3 | end-of-treatment | HPV-16 | 113824 | 9 | 175 | 135 | 40 | 0.04 |
| P5 | end-of-treatment | HPV-16 | 144870 | 3 | 174 | 133 | 41 | <1 × 10⁻³²⁰ |

Groups
HPV

| | | | | | | |
|---|---|---|---|---|---|---|
| HPV- | 842787 | 3893 | 169 | 153 | 16 | <1 × 10⁻³²⁰ |
| HPV- | 336014 | 12004 | 168 | 144 | 22 | <1 × 10⁻³²⁰ |

Timep

| | | | | | | |
|---|---|---|---|---|---|---|
| end-of-treatment | 827602 | 13803 | 169 | 146 | 23 | <1 × 10⁻³²⁰ |
| RECR | 351199 | 2094 | 169 | 150 | 19 | 1.8 × 10⁻⁸⁶ |

Panel

| | | | | | | |
|---|---|---|---|---|---|---|
| Single-strand | 336014 | 12004 | 168 | 144 | 22 | <1 × 10⁻³²⁰ |
| Dual-strand | 842787 | 3893 | 169 | 153 | 16 | <1 × 10⁻³²⁰ |

Abbreviation:
3MFU = 3 month post-treatment follow up;
RECR = recurrence

TABLE 5
38 HPV genomes interrogated by the HPV genotyping panel

| HPV genome | HPV name |
|---|---|
| gi\|333071 | Human papillomavirus 5 (HPV-5) |
| gi\|60955 | Human papillomavirus 6 (HPV-6) |
| gi\|333026 | Human papillomavirus 11 (HPV-11) |
| gi\|396910 | Human papillomavirus 12 (HPV-12) |
| gi\|333031 | Human papillomavirus 16 (HPV-16) |
| gi\|60975 | Human papillomavirus 18 (HPV-18) |
| gi\|9627305 | Human papillomavirus 26 (HPV-26) |
| gi\|333048 | Human papillomavirus 31 (HPV-31) |
| gi\|333049 | Human papillomavirus 33 (HPV-33) |
| gi\|396997 | Human papillomavirus 35 (HPV-35) |
| gi\|1020234 | Human papillomavirus 38 (HPV-38) |
| gi\|333245 | Human papillomavirus 39 (HPV-39) |
| gi\|397014 | Human papillomavirus 40 (HPV-40) |
| gi\|333211 | Human papillomavirus 42 (HPV-42) |
| gi\|397022 | Human papillomavirus 45 (HPV-45) |
| gi\|333087 | Human papillomavirus 51 (HPV-51) |
| gi\|397038 | Human papillomavirus 52 (HPV-52) |
| gi\|9627377 | Human papillomavirus 53 (HPV-53) |
| gi\|9628437 | Human papillomavirus 54 (HPV-54) |
| gi\|396937660 | Human papillomavirus 55 (HPV-55) |
| gi\|397053 | Human papillomavirus 56 (HPV-56) |
| gi\|222386 | Human papillomavirus 58 (HPV-58) |
| gi\|557236 | Human papillomavirus 59 (HPV-59) |
| gi\|9628574 | Human papillomavirus 61 (HPV-61) |
| gi\|39932599 | Human papillomavirus 62 (HPV-62) |
| gi\|1020290 | Human papillomavirus 66 (HPV-66) |
| gi\|3228267 | Human papillomavirus 67 (HPV-67) |
| gi\|71726685 | Human papillomavirus 68 (HPV-68) |
| gi\|6970418 | Human papillomavirus 69 (HPV-69) |
| gi\|1173493 | Human papillomavirus 70 (HPV-70) |
| gi\|12084981 | Human papillomavirus 71 (HPV-71) |
| gi\|1491683 | Human papillomavirus 72 (HPV-72) |
| gi\|1491692 | Human papillomavirus 73 (HPV-73) |
| gi\|40804509 | Human papillomavirus 81 (HPV-81) |
| gi\|6970427 | Human papillomavirus 82 (HPV-82) |
| gi\|5059324 | Human papillomavirus 83 (HPV-83) |
| gi\|12958167 | Human papillomavirus 84 (HPV-84) |
| gi\|50253426 | Human papillomavirus 96 (HPV-96) |

| E6E7Genotyping | | Length | SEQ |
|---|---|---|---|
| Panel_SequenceName | Sequence | Length | ID#: |
| gi\|020234.E6\| | ACCCTGTAGATTCTGCAACAGTT | 120 | 1 |
| lcl\|HPV38_E6.1 | TCCTCACGTACATTGAATTACGT | | |
| Human papillomavirus | GAGTTTGATTACAAGAACTTACA | | |
| 38 (HPV38), | GTTAATCTGGACTCAAGA | | |
| E6 gene_1 | GGATTTTGTTTTTGCATGTTGTA | | |
| | GCAGTTGTGC | | |
| gi\|1020234.E6\| | GCAACAGACAATAGGCCTTATTG | 120 | 2 |
| lcl\|HPV38_E6.1\| | TTATAAGGTGTCAGTATTGTTTA | | |
| Human papillomavirus | AAGTGTCTTGATTTGATAGAAAA | | |
| 38 (HPV38), | ATTAGATATCTGTTGCTCT | | |
| E6 gene_2 | CATCAAGCATTTCACAAGGTTAG | | |
| | AGGCAATTG | | |
| gi\|1020290.E6\| | TTTACTTGATCTTAGATTATCAT | 120 | 3 |
| lcl\|HPV66_E6.1 | GTGTATACTGCAAAAAGGAACTT | | |
| Human papillomavirus | ACAAGTTTAGAGCTATATAGGTT | | |
| 66 (HPV66), | TGCATGTATTGAGTTAAAAC | | |
| E6 gene_1 | TAGTATATAGAAACAATTGGCCA | | |
| | TATGCAGT | | |
| gi\|1020290.E6\| | ATTAGAAAGTATAACTAAAAAAC | 120 | 4 |
| lcl\|HPV66_E6.1 | AGTTATCTGATTTATCAATAAGG | | |
| Human papillomavirus | TGCTACCGATGTCAATGTCCGTT | | |
| 66 (HPV66), | AACACCGGAGGAAAAACA | | |
| E6 gene_2 | ATTGCACTGTGAACATAAAAGAC | | |
| | GATTTCATTA | | |
| gi\|1173493.E6\| | GCACGACATTACAATAGACTGTG | 120 | 5 |
| lcl\|HPV70_E6.1 | TCTATTGTAAAACACAGCTACAG | | |
| Human papillomavirus | CAAACAGAGGTATATGAATTTGC | | |
| 70 (HPV70), | ATTTAGTGATTTATTTATA | | |
| E6 gene_1 | GTATATAGAAACGGGGAGCCATA | | |
| | TGCTGCATG | | |
| gi\|1173493.E6\| | GGAAAGCATAACTAATACCAAGT | 120 | 6 |
| lcl\|HPv70_E6.1\| | TATATAATTTATCAATAAGGTGC | | |
| Human papillomavirus | ATGAGTTGCCTGAAACCATTGTG | | |
| 70 (HPV70), | TCCAGCAGAAAAATTAAG | | |
| E6 gene_2 | GCATGTTAATACCAAAAGAAGAT | | |
| | TTCACCAAAT | | |
| gi\|12084981.E6\| | GTACGACGTGGACCTGCAGGACC | 120 | 7 |
| lcl\|HPV71_E6.1\| | TGAACCTAACCTGCATATTCTGC | | |
| Human papillomavirus | AGAAATCAATTAACAGACGTGGA | | |
| 71 (HPV71), | AGTCGTGGCCTTTGCAT | | |
| E6 gene_1 | ATAAGGAGCTGAAAGTTGTGTGG | | |
| | AGAAGTGGTTT | | |
| gi\|12084981.E6\| | CTTTGCAAACACAGTGGAATTAG | 120 | 8 |
| lcl\|HPV71_E6.1\| | ACACCGGAACGCCAGTTACAGAG | | |
| Human papillomavirus | CAACTAATACGGTGCTACGTGTG | | |
| 71 (HPV71), | TCACAAGCCATTGTGTA | | |
| E6 gene_2 | GTGTGGAAAAAGAAAGAATAATT | | |
| | ACAGAAGGCAG | | |
| gi\|12958167.E6\| | ACGATTAATTTGCATCTTTTGCA | 120 | 9 |
| lcl\|HPV84_E6.1\| | AGGAAGAATTAACGGAAGGCGAA | | |
| Human papillomavirus | GTGCTGGCCTTTGCAGTAAAGGA | | |
| 84 (HPV84), | ATTACTAATTGTTTGGAG | | |
| E6 gene_1 | GTATAATTTCCCTCATGGGGTGT | | |
| | GCATGAAATG | | |
| gi\|12958167.E6\| | AACAGGATTACCACTTGCACAAA | 120 | 10 |
| lcl\|HPV84_E6.1\| | TAAATATAAGGTGCCACGCGTGC | | |
| Human papillomavirus | TGCAAGCCATTGTGCTATCAGGA | | |
| 84 (HPV84), | AAAGGAGTATATGGTGG | | |
| E6 gene_2 | AATTGCAGTTGCTATTCCACAAA | | |
| | ATAGCTGGACA | | |
| gi\|1491683.E6\| | ACGGATTACCTGCATATTTTGCA | 120 | 11 |
| lcl\|HPV72_E6.1\| | AAAATGAATTAACAACAGAAGAA | | |
| Human papillomavirus | TTGCTGGCGATTGCAATAAAGGA | | |
| 72 (HPV72), | GCTGCAGATTGTGTGGC | | |
| E6 gene_1 | GGGACAACTGGCCATTTGGAGTC | | |
| | TGCGCACCATG | | |
| gi\|1491683.E6\| | AACAGGCAAATCATTAGCAGAAC | 120 | 12 |
| lcl\|HPV72_E6.1 | TATATATAAGGTGCCATGCATGC | | |
| Human papillomavirus | TGCAAACCCCTAAGCTGTCAGGA | | |
| 72 (HPV72), | AAAGGAATATCAGGTGC | | |
| E6 gene_2 | AGACAGGAATCCACTTCCACAAG | | |
| | ATAAGCGGACT | | |
| gi\|1491692.E6\| | TGATATAAACCTGGACTGTGTGT | 120 | 13 |
| lcl\|HPV73_E6.1\| | TTTGCCAACGTGGACTGTACAGA | | |
| Human papillomavirus | TCTGAGGTATATGATTTTGCATT | | |
| 73 (HPV73), | TAGTGATTTGTGTATTGTA | | |
| E6 gene_1 | TATAGAAAGGATAAACCATATGG | | |
| | TGTATGTCA | | |
| gi\|1491692.E6\| | AAATTTAACTAACAAACAGTTAT | 120 | 14 |
| lcl\|HPV73_E6.1 | GTAATATTTTAATAAGGTGCGGA | | |
| Human papillomavirus | AAATGCCAAAAACCATTATGTCC | | |
| 73 (HPV73), | ACTGGAAAAGCAAAAGCAT | | |
| E6 gene_2 | GTAGATGAAAAAAAACGGTTTCA | | |
| | TCAAATAGC | | |
| gi\|222386.E6\| | AATCGAATTGAAATGCGTTGAAT | 120 | 15 |
| lcl\|HPV58_E6.1\| | GCAAAAAGACTTTGCAGCGATCT | | |
| Human papillomavirus | GAGGTATATGACTTTGTATTTGC | | |
| 58 (HPV58), | AGATTTAAGAATAGTGTAT | | |
| E6 gene_1 | AGAGATGGAAATCCATTTGCAGT | | |
| | ATGTAAAGT | | |
| gi\|222386.E6\| | AACACTAAAAAAGTGTTTAAATG | 120 | 16 |
| lcl\|HPV58_E6.1\| | AAATATTAATTAGATGTATTATT | | |
| Human papillomavirus | TGTCAAAGACCATTGTGTCCACA | | |
| 58 (HPV58), | AGAAAAAAAAAGGCATGTG | | |
| E6 gene_2 | GATTTAAACAAAAGGTTTCATAA | | |
| | TATTTCGGG | | |
| gi\|3228267.E6\| | AATAAGTTTGCCCTGCGTGCAGT | 120 | 17 |
| lcl\|HPV67_E6.1 | GCAAAAAAACTTTGGACAGAAAC | | |
| Human papillomavirus | GAGGTATATGACTTTTTATTTAC | | |
| 67 (HPV67), | TGATTTGAAAATAGTATAC | | |
| E6 gene_1 | AGATGTGGGAATCCGTATGGGGT | | |
| | ATGTAAGCA | | |
| gi\|3228267.E6\| | AATTGTGCATAAACCATTAAATG | 120 | 18 |
| lcl\|HPV67_E6.1 | AAATTACAATACGATGTATAACA | | |
| Human papillomavirus | TGTCAAAGACCATTGTGTCCACA | | |
| 67 (HPV67), | AGAAAAGCAAAGGCATGT | | |
| E6 gene_2 | TGATCGCAAAAAGCGATTTCATA | | |
| | ATATTTCCAA | | |
| gi\|333026.E6\| | CACTCTGCAAATTCAGTGCGTGT | 120 | 19 |
| lcl\|HPV11_E6.1\| | TTTGCAGGAATGCACTGACCACC | | |
| Human papillomavirus | GCAGAGATATATGCATATGCCTA | | |
| 11 (HPV11), | TAAGAACCTAAAGGTTG | | |
| E6 gene_1 | TGTGGCGAGACAACTTTCCCTTT | | |
| | GCAGCGTGTGC | | |
| gi\|333026.E6\| | AGAAGAAACCAATGAAGATATTT | 120 | 20 |
| lcl\|HPV11_E6.1\| | TAAAAGTGTTAATTCGTTGTTAC | | |
| Human papillomavirus | CTGTGTCACAAGCCGTTGTGTGA | | |
| 11 (HPV11), | AATAGAAAAACTAAAGCA | | |
| E6 gene_2 | CATATTGGGAAAGGCACGCTTCA | | |
| | TAAAACTAAA | | |
| gi\|333031 E6\| | GCTGCAAACAACTATACATGATA | 120 | 21 |
| lcl\|HPV16_E6.1\| | TAATATTAGAATGTGTGTACTGC | | |
| Human papillomavirus | AAGCAACAGTTACTGCGACGTGA | | |
| 16 (HPV16), | GGTATATGACTTTGCTTTT | | |
| E6 gene_1 | CGGGATTTATGCATAGTATATAG | | |
| | AGATGGGAA | | |
| gi\|333031.E6\| | GTATGGAACAACATTAGAACAGC | 120 | 22 |
| lcl\|HPV16_E6.1\| | AATACAACAAACCGTTGTGTGAT | | |
| Human papillomavirus | TTGTTAATTAGGTGTATTAACTG | | |
| 16 (HPV16), | TCAAAAGCCACTGTGTCC | | |
| E6 gene_2 | TGAAGAAAAGCAAAGACATCTGG | | |
| | ACAAAAAGCA | | |
| gi\|333048.E6\| | ACTAAGATTGAATTGTGTCTACT | 120 | 23 |
| lcl\|HPV31_E6.1\| | GCAAAGGTCAGTTAACAGAAACA | | |
| Human papillomavirus | GAGGTATTAGATTTTGCATTTAC | | |
| 31 (HPV31), | AGATTTAACAATAGTATAT | | |
| E6 gene_1 | AGGGACGACACACCACACGGAGT | | |
| | GTGTACAAA | | |
| gi\|333048.E6\| | ATTGACAAACAAAGGTATATGTG | 120 | 24 |
| lcl\|HPV31_E6.1\| | ATTTGTTAATTAGGTGTATAACG | | |
| Human papillomavirus | TGTCAAAGACCGTTGTGTCCAGA | | |
| 31 (HPV31), | AGAAAAACAAAGACATTT | | |
| E6 gene_2 | GGATAAAAAGAAACGATTCCACA | | |
| | ACATAGGAGG | | |
| gi\|333049.E6\| | CATTGAACTACAGTGCGTGGAAT | 120 | 25 |
| lcl\|HPV33_E6.1\| | GCAAAAAACCTTTGCAACGATCT | | |
| Human papillomavirus | GAGGTATATGATTTTGCATTTGC | | |
| 33 (HPV33), | AGATTTAACAGTTGTATAT | | |
| E6 gene_1 | AGAGAGGGAAATCCATTTGGAAT | | |
| | ATGTAAACT | | |
| gi\|333049.E6\| | AACAGTTAAAAAACCTTTAAATG | 120 | 26 |
| lcl\|HPV33_E6.1\| | AAATATTAATTAGGTGTATTATA | | |
| Human papillomavirus | TGTCAAAGACCTTTGTGTCCTCA | | |
| 33 (HPV33), | AGAAAAAAAACGACATGTG | | |
| E6 gene_2 | GATTTAAACAAACGATTTCATAA | | |
| | TATTTCGGG | | |
| gi\|333071.E6\| | CTTAGCTGAAGCCTTAGGCATCC | 120 | 27 |
| lcl\|HPV5_E6.1\| | CTGTGATTGATTGTTTAATACCT | | |
| Human papillomavirus | TGCAATTTCTGTGGCAACTTTCT | | |
| 5 (HPV5), | AAATTATTTGGAAGCTTGT | | |
| E6 gene_1 | GAATTCGACTACAAAAGGCTTAG | | |
| | TCTAATTTG | | |
| gi\|333071.E6\| | TGAGCAGACAGTGTTAGGAAGAG | 120 | 28 |
| lcl\|HPV5_E6.1\| | ATATTGAATTAGCTTCAGGACTT | | |
| Human papillomavirus | TCAATATTTGATATTGATATCAG | | |
| 5 (HPV5), | GTGTCAAACTTGCTTAGCA | | |
| E6 gene_2 | TTTCTTGACATTATAGAAAAGTT | | |
| | AGATTGCTG | | |
| gi\|333087.E6\| | TATACAGGTAGTGTGTGTGTATT | 120 | 29 |
| lcl\|HPV51_E6.1\| | GTAAAAAGGAATTATGTAGAGCA | | |
| Human papillomavirus | GATGTATATAATGTAGCATTTAC | | |
| 51 (HPV51), | TGAAATTAAGATTGTATATA | | |
| E6 gene_1 | GGGATAATAATCCATATGCAGTA | | |
| | TGCAAACA | | |
| gil333087.E6\| | AATTACTAAAAAAAGCTTATATG | 120 | 30 |
| lcl\|HPV51_E6.1 | ATTTATCGATAAGGTGTCATAGA | | |
| Human papillomavirus | TGTCAAAGACCACTTGGGCCTGA | | |
| 51 (HPV51), | AGAAAAGCAAAAATTGGT | | |
| E6 gene_2 | GGACGAAAAAAAAAGGTTCCATG | | |
| | AAATAGCGGG | | |
| gi\|333211 .E6\| | GAATTTACAGATTTCCTGCATTT | 120 | 31 |
| lcl\|HPV42_E6.1\| | GGTGCAAAAAGCACTTAACAGGC | | |
| Human papillomavirus | GCAGAGGTGCTCGCGTACCATTT | | |
| 42 (HPV42), | TAAAGATTTGGTAGTGGT | | |
| E6 gene_1 | GTGGAGGAAGGACTTTCCATATG | | |
| | CTGCATGTGC | | |
| gi\|333211.E6\| | GAAAGAAACTGGACTACTTTTAG | 120 | 32 |
| lcl\|HPV42_E6.1\| | AAGAACAACAAATTAGATGTGCC | | |
| Human papillomavirus | TTGTGTCAAAAGCCGTTATCACA | | |
| 42 (HPV42), | GAGCGAAAAAAACCATCA | | |
| E6 gene_2 | TATTGATACAGGTACAAGATTTC | | |
| | AATTTATATT | | |
| gi\|333245.E6\| | GCAGGACATTACAATAGCCTGTG | 120 | 33 |
| lcl\|HPV39_E6.1\| | TCTATTGCAGACGACCACTACAG | | |
| Human papillomavirus | CAAACCGAGGTATATGAATTTGC | | |
| 39 (HPV39), | ATTTAGTGATTTATATGT | | |
| E6 gene_1 | AGTATATAGGGACGGGGAACCAC | | |
| | TAGCTGCATG | | |
| gi\|333245.E6\| | AGAAAATATAACTAATACAAAGT | 120 | 34 |
| lcl\|HPV39_E6.1\| | TATATAATTTATTAATAAGGTGC | | |
| Human papillomavirus | ATGTGTTGTCTGAAACCGCTGTG | | |
| 39 (HPV39), | TCCAGCAGAAAAATTAAGA | | |
| E6 gene_2 | CACCTAAATAGCAAACGAAGATT | | |
| | TCATAAAAT | | |
| gi\|396910.E6\| | TGACCTTTTAGATATACCTTTAG | 120 | 35 |
| lcl\|HPV12_E6.1\| | TTGACTGTTTGGTACCTTGCAAT | | |
| Human papillomavirus | TTTTGCGGAAAGTTCTTAGATTT | | |
| 12 (HPV12), | TCTGGAAGTGTGTGATTTTG | | |
| E6 gene_1 | ACAAAAAGCAGCTAACACTAATT | | |
| | TGGAAAGG | | |
| gi\|39691O.E6\| | AACAGTGCTAGGTAGAGATATAG | 120 | 36 |
| lcl\|HPV12_E6.1\| | AGCTTGCTACTGGAAAATCTATA | | |
| Human papillomavirus | TTTGACTTAAAGATAAGGTGTCA | | |
| 12 (HPV12), | GACGTGCTTGTCATTTTTA | | |
| E6 gene_2 | GATACAATTGAAAAGTTAGACAG | | |
| | CTGTGGTCG | | |
| gi\|396937660\|emb\| | CCATTTGCAAATTTTATGCGTGT | 120 | 37 |
| HE963220.11 | TTTGCAGAAAAACCTTGACCACT | | |
| Human papillomavirus | GCAGAGGTTTATTCATTCGCATA | | |
| type 55 | TAAACAGTTATATGTAGTG | | |
| (HPV55) E6 gene, | TTTCGAGGTAACTTTCCATTTGC | | |
| isolate CAC1126-10_1 | AGCCTGTGC | | |
| gi\|396937660\|emb\| | AGAAGAGACAAAGCAGTCAATTT | 120 | 38 |
| HE963220.11 | TGGACGTGTTCATACGCTGTTAT | | |
| Human papillomavirus | TTGTGCCACAAACCATTGTGCCA | | |
| type 55 | CGTGGAAAAGGTGCGCC | | |
| (HPV55) E6 gene, | ACATATTGGAGAAGGCGCGATTT | | |
| isolate CAC1126-10_2 | ATTAAATTACA | | |
| gi\|396997.E6\| | AATTTGTTTGAATTGTGTATACT | 120 | 39 |
| lcl\|HPV35_E6.1\| | GCAAACAAGAATTACAGCGGAGT | | |
| Human papillomavirus | GAGGTATATGACTTTGCATGCTA | | |
| 35 (HPV35), | TGATTTGTGTATAGTATAT | | |
| E6 gene_1 | AGAGAAGGCCAGCCATATGGAGT | | |
| | ATGCATGAA | | |
| gi\|396997.E6\| | ACAATGCAACAAACAGTTATGTC | 120 | 40 |
| lcl\|HPV35_E6.1\| | ATTTATTAATTAGGTGTATTACA | | |
| Human papillomavirus | TGTCAAAAACCGCTGTGTCCAGT | | |
| 35 (HPV35), | TGAAAAGCAAAGACATTTA | | |
| E6 gene_2 | GAAGAAAAAAAACGATTCCATAA | | |
| | CATCGGTGG | | |
| gi\|397014.E6\| | GTTGCAAATTGATTGTGTGTTTT | 120 | 41 |
| lcl\|HPV40_E6.1\| | GCAAGACGGTCCTAAAAACAGCT | | |
| Human papillomavirus | GAGGTACTGGCCTTTGCCTTTAG | | |
| 40 (HPV40), | AGAGTTATATGTTGTGTG | | |
| E6 gene_1 | GCGCGACGACTTTCCACACGCCG | | |
| | CATGTCCACG | | |
| gi\|397014.E6\| | AGAGACAGGATTAACCATTTTAC | 120 | 42 |
| lcl\|HPV40_E6.1\| | AAGTAAGGATTAGATGCTGCAAG | | |
| Human papillomavirus | TGCCACAAGCCTTTGTCTCCCGT | | |
| 40 (HPV40), | GGAAAAAACCAACCATA | | |
| E6 gene_2 | TTGTAAAGAAGACGCAATTCTTT | | |
| | AAATTAAAAGA | | |
| gi\|397022.E6\| | ACAAGACGTATCTATTGCCTGTG | 120 | 43 |
| lcl\|HPV45_E6.1\| | TATATTGCAAAGCAACATTGGAA | | |
| Human papillomavirus | CGCACAGAGGTATATCAATTTGC | | |
| 45 (HPV45), | TTTTAAAGATTTATGTATA | | |
| E6 gene_1 | GTGTATAGAGACTGTATAGCATA | | |
| | TGCTGCATG | | |
| gi\|397022.E6\| | GGAAAAAATAACTAATACAGAGT | 120 | 44 |
| lcl\|HPV45_E6.1\| | TGTATAATTTGTTAATAAGGTGC | | |
| Human papillomavirus | CTGCGGTGCCAGAAACCATTGAA | | |
| 45 (HPV45), | CCCAGCAGAAAAACGTA | | |
| E6 gene_2 | GACACCTTAAGGACAAACGAAGA | | |
| | TTTCACAGCAT | | |
| gi\|397038.E6\| | AATAAGGCTGCAGTGTGTGCAGT | 120 | 45 |
| lcl\|HPV52_E6.1\| | GCAAAAAAGAGCTACAACGAAGA | | |
| Human papillomavirus | GAGGTATACAAGTTTCTATTTAC | | |
| 52 (HPV52), | AGATTTACGAATAGTATA | | |
| E6 gene_1 | TAGAGACAATAATCCATATGGCG | | |
| | TGTGTATTAT | | |
| gi\|397038.E6\| | GAGGGTAAAAAAACCATTAAGTG | 120 | 46 |
| lcl\|HPV52_E6.1\| | AAATAACTATTAGATGTATAATT | | |
| Human papillomavirus | TGTCAAACGCCATTATGTCCTGA | | |
| 52 (HPV52), | AGAAAAAGAAAGACATGTT | | |
| E6 gene_2 | AATGCAAACAAGCGATTTCATAA | | |
| | TATTATGGG | | |
| gi\|397053.E6\| | TTTAATTGATCTTAGATTATCAT | 120 | 47 |
| lcl\|HPV56_E6.1\| | GTGTATATTGCAAAAAAGAACTA | | |
| Human papillomavirus | ACACGTGCTGAGGTATATAATTT | | |
| 56 (HPV56), | TGCATGCACTGAATTAAAAT | | |
| E6 gene_1 | TAGTGTATAGGGATGATTTTCCT | | |
| | TATGCAGT | | |
| gi\|397053.E6\| | ACTAGAAAGTATAACTAAAAAAC | 120 | 48 |
| lcl\|HPV56_E6.1\| | AGTTATGTGATTTATTAATAAGG | | |
| Human papillomavirus | TGCTACAGATGTCAAAGTCCGTT | | |
| 56 (HPV56), | AACTCCGGAGGAAAAGCA | | |
| E6 gene_2 | ATTGCATTGTGACAGAAAAAGAC | | |
| | GATTTCATCT | | |
| gi\|39932599.E6\|lcl\| | GCACCTAACCTGCATATTTTGCA | 120 | 49 |
| HPV62_E6.1\| | AAACAGACCTGTCCGCCGGTGAA | | |
| Human papillomavirus | CTGCTGTCCTTTGCAATACGGGA | | |
| 62 (HPV62), | GCTACACGTGGTGCGG | | |
| E6 gene_1 | CGAAAGGACTGGCCATTTGGAGT | | |
| | ATGCGCAGCATG | | |
| gi\|39932599.E6\| | AACAGGCAGTCCGCTTGCACAAA | 120 | 50 |
| lcl\|HPV62_E6.1\| | TATATATTAGATGCCATGCATGC | | |
| Human papillomavirus | TGTATGCCATTAACCTGTCAGGA | | |
| 62 (HPV62), | AAAGGAGTATCAGGTAG | | |
| E6 gene_2 | AGAGGGGAGTGCACTTCCACAAA | | |
| | ATAGCAGGAGT | | |
| gi\|40804509.E6\| | AGTGGACCTAGACGACCTGCAAC | 120 | 51 |
| lcl\|HPV81_E6.1\| | TGACCTGCATATTTTGCAAAAAG | | |
| Human papillomavirus | GAATTAACAGTAGGTGAATTGCT | | |
| 81 (HPV81), | GTCCTTTGCAATAAGAG | | |
| E6 gene_1 | AGCTAAACTTGGTGTGGCGAAAG | | |
| | AACTGGCCATT | | |
| gi\|40804509.E6\| | ACCAACAGTAGAAGAGGAGACAG | 120 | 52 |
| lcl\|HPV81_E6.1\| | GGCAACCGCTTGCACAAATATAT | | |
| Human papillomavirus | ATACGATGCCATGCATGCTATAA | | |
| 81 (HPV81), | ACCGTTAACATACCAAG | | |
| E6 gene_2 | AAAAGGAGTATCTTGTGACAGGC | | |
| | CTAATCCACTT | | |
| gi\|50253426.E6\| | CTTCTGTAAGAAATTTTTAACCT | 120 | 53 |
| lcl\|HPV96_E6.1\| | TTGAGGAGCTATTAGGATTTGAT | | |
| Human papillomavirus | AGCAAGACTTTAAATCTTATTTG | | |
| 96 (HPV96), | GAAGGAAGGTTATGCATAT | | |
| E6 gene_1 | GCGTGTTGTTTCAATTGTGCTAG | | |
| | AACAGTAGC | | |
| gi\|50253426.E6\| | TTGTTGTATTGTTGTTAGATGCC | 120 | 54 |
| lcl\|HPV96_E6.1\| | AGTTTTGTTTGCGACTGTTAGAT | | |
| Human papillomavirus | TATTTAGAAAAGTTAAATATTTG | | |
| 96 (HPV96), | TGGGTATCAAGAAAACTTTT | | |
| E6 gene_2 | ATTTAGTAAGAGGAGGTTGGAAA | | |
| | GGCATTTG | | |
| gi\|5059324.E6\| | ACTGATTTGCATTTATTGCACAA | 120 | 55 |
| lcl\|HPV83_E6.1\| | ATGAATTAACAACAGCAGAAGTG | | |
| Human papillomavirus | CTGTCCTTTGCATGGAAGGAGCT | | |
| 83 (HPV83), | GTGTATTAAGTGGGACCA | | |
| E6 gene_1 | CGAACTGCCCTACGGAGCGTGTG | | |
| | CACAGTGTCT | | |
| gi\|5059324.E6\| | AAAAACACCACTGGCACAGCTGT | 120 | 56 |
| lcl\|HPV83_E6.1\| | ATATACGATGCCATATGTGCCTG | | |
| Human papillomavirus | AAGCCGTTGAGTAGTCAAGAAAA | | |
| 83 (HPV83), | AGAATATTTGGTGCAAA | | |
| E6 gene_2 | CAGGTGATAGACTTCATAATATA | | |
| | GCAGGACAGTG | | |
| gi\|557236.E6\| | GCATGATATTCGCATCAATTGTG | 120 | 57 |
| lcl\|HPV59_E6.1\| | TGTTTTGCAAAGGGGAACTGCAA | | |
| Human papillomavirus | GAAAGAGAGGTATTTGAATTTGC | | |
| 59 (HPV59), | TTTTAATGACTTATTTATA | | |
| E6 gene_1 | GTGTATAGAGACTGTACACCGTA | | |
| | TGCAGCGTG | | |
| gi\|557236.E6\| | AGAGGCTGAAACCAAGACACCGT | 120 | 58 |
| lcl\|HPV59_E6.1\| | TACATGAGCTGCTGATACGCTGT | | |
| Human papillomavirus | TATAGATGCCTAAAACCTCTATG | | |
| 59 (HPV59), | TCCAACAGATAAATTAA | | |
| E6 gene_2 | AGCATATAACTGAAAAAAGAAGA | | |
| | TTCCATAATAT | | |
| gi\|60955.E6\|lcl\| | TACGTTGCAAATTAATTGTGTGT | 120 | 59 |
| HPV6_E6.1\| | TTTGCAAGAATGCACTGACCACA | | |
| Human papillomavirus | GCAGAGATTTATTCATATGCATA | | |
| 6 (HPV6), | TAAACACCTAAAGGTCCTG | | |
| E6 gene_1 | TTTCGAGGCGGCTATCCATATGC | | |
| | AGCCTGCGC | | |
| gil60955.E6\| | AGAAGAAACTAAACAAGACATCT | 120 | 60 |
| lcl\|HPV6_E6.1\| | TAGACGTGCTAATTCGGTGCTAC | | |
| Human papillomavirus | CTGTGTCACAAACCGCTGTGTGA | | |
| 6 (HPV6), | AGTAGAAAAGGTAAAAC | | |
| E6 gene_2 | ATATACTAACCAAGGCGCGGTTC | | |
| | ATAAAGCTAAA | | |
| gi\|60975.E6\| | GCAAGACATAGAAATAACCTGTG | 120 | 61 |
| lcl\|HPV18_E6.1\| | TATATTGCAAGACAGTATTGGAA | | |
| Human papillomavirus | CTTACAGAGGTATTTGAATTTGC | | |
| 18 (HPV18), | ATTTAAAGATTTATTTGTG | | |
| E6 gene_1 | GTGTATAGAGACAGTATACCGCA | | |
| | TGCTGCATG | | |
| gi\|60975.E6\| | GGAAAAACTAACTAACACTGGGT | 120 | 62 |
| lcl\|HPV18_E6.1\| | TATACAATTTATTAATAAGGTGC | | |
| Human papillomavirus | CTGCGGTGCCAGAAACCGTTGAA | | |
| 18 (HPV18), | TCCAGCAGAAAAACTTA | | |
| E6 gene_2 | GACACCTTAATGAAAAACGACGA | | |
| | TTTCACAACAT | | |
| gi\|6970418.E6\| | TTTGCAGGTACAGTGTGTATATT | 120 | 63 |
| lcl\|HPV69_E6.1 | GCAAGAAAACATTAGAATGGGCA | | |
| Human papillomavirus | GATGTATATAACTTTGCAATATG | | |
| 69 (HPV69), | TGATTTAAGAATAGTGTAT | | |
| E6 gene_1 | AGAAATGATAGTGCATATGGTGC | | |
| | ATGTAAAAA | | |
| gi\|6970418.E6\| | GCGTCCTAAACGAAGTTTGTGTA | 120 | 64 |
| lcl\|HPV69_E6.1 | ATTTGTTAATAAGGTGTCATAGA | | |
| Human papillomavirus | TGCCAAATACCATTGGGACCGGA | | |
| 69 (HPV69), | AGAAAAACAGAGAATTGT | | |
| E6 gene_2 | GGATGAAAAGCGACGGTTCCATG | | |
| | AAATAGCAGG | | |
| gi\|6970427.E6\| | TATTCAGGTATTGTGTGTATATT | 120 | 65 |
| lcl\|HPV82_E6.1 | GTAAAAAGGAGTTGTGTAGAGCA | | |
| Human papillomavirus | GATGTGTATAATGTAGCATTTAC | | |
| 82 (HPV82), | AGAACTTAGGATTGTATAT | | |
| E6 gene_1 | AGGGACAATACGCCATATGCAGC | | |
| | ATGCAAAAA | | |
| gi\|6970427.E6\| | CATTACTAACAAAAGTTTATATG | 120 | 66 |
| lcl\|HPV82_E6.1 | AATTATTAATAAGGTGTCATAGA | | |
| Human papillomavirus | TGTCAGAGACCACTTGGGCCTGA | | |
| 82 (HPV82), | AGAAAAGCAAAAGGTGGT | | |
| E6 gene_2 | GGACGACAAAAAAAGGTTTCATG | | |
| | AAATAGCGGG | | |
| gi\|71726685.E6\| | GCATGACGTTACAATAGACTGTG | 120 | 67 |
| lcl\|HPV68_E6.1\| | TCTATTGCAGAAGGCAACTACAA | | |
| Human papillomavirus | CGGACAGAGGTATATGAATTTGC | | |
| 68 (HPV68), | CTTTAGTGACCTATGTGT | | |
| E6 gene_1 | AGTGTATAGAGACGGGGTACCAT | | |
| | TTGCTGCATG | | |
| gi\|71726685.E6\| | AGAAACCATAACTAATACAAAGT | 120 | 68 |
| lcl\|HPV68_E6.1\| | TATATAATTTATTGATAAGGTGC | | |
| Human papillomavirus | ATGAGTTGCCTGAAACCATTGTG | | |
| 68 (HPV68), | TCCAGCAGAAAAACTAAG | | |
| E6 gene_2 | GCACCTAACAACAAAACGAAGAT | | |
| | TACATAAAAT | | |
| gi\|9627305.E6\| | TTTGCAGGTACAGTGTGTATATT | 120 | 69 |
| lcl\|HPV26_E6.1\| | GCAAGGAAACCTTACAATGGGCT | | |
| Human papillomavirus | GATGTATATAATTTTGCAATTTG | | |
| 26 (HPV26), | TGACCTAAGAGTAGTATAT | | |
| E6 gene_1 | AGAGATAGGAGTCCGTATGCTGC | | |
| | ATGCAAAAG | | |
| gi\|9627305.E6\| | CTTAACTAAAAAAAGTTTATGTA | 120 | 70 |
| lcl\|HPV26_E6.1 | ATTTGTTAATAAGGTGTCATAGA | | |
| Human papillomavirus | TGTCAAATGCCATTGGGGCCAGA | | |
| 26 (HPV26), | AGAAAAACAAAGAATTGTG | | |
| E6 gene_2 | GATGAAAAGCGACGATTTCACGA | | |
| | AATAGCAGG | | |
| gi\|9627377.E6\| | ACCATTGCTGGAGCTGCAACTTG | 120 | 71 |
| lcl\|HPV53_E6.1\| | GCTGTGTGTTCTGCAAGAAGGCA | | |
| Human papillomavirus | TTGACAGCGTCAGAGGTATATAA | | |
| 53 (HPV53), | TTTTGCATATACAGATCT | | |
| E6 gene_1 | AAGAGTAGTGTATAGAGACGGGT | | |
| | ATCCGTATGG | | |
| gi\|9627377.E6\| | TAGCCTGGAAGCACTAACTAAAA | 120 | 72 |
| lcl\|HPV53_E6.1 | AAAAGTTATCTGATTTATCAATA | | |
| Human papillomavirus | AGGTGCTACAGATGTCAACATCC | | |
| 53 (HPV53), | GTTGACACCAGAGGAAAA | | |
| E6 gene_2 | ACAGTTACACTGTGACTATAAGA | | |
| | AACGGTTTCA | | |
| gi\|9628437.E6\| | CAGTTATTTGGGGGCAATGTCTG | 120 | 73 |
| lcl\|HPV54_E6.1\| | CTACTGAACCCCACACGGACCAG | | |
| Human papillomavirus | CCGCGTACTCTAGCTGATTTGTG | | |
| 54 (HPV54), | CAAGGTATGCAATATTC | | |
| E6 gene_1 | CTATGCATAGTTTGCAACTTCCT | | |
| | TGTGCCTTTTG | | |
| gi\|9628437.E6\| | TCCACATGCTGCATGTGCACTGT | 120 | 74 |
| lcl\|HPV54_E6.1 | GCCTAGAACTGCACGGGCAAATA | | |
| Human papillomavirus | AATTATAGAAGGCATCGCGACCG | | |
| 54 (HPV54), | TGCGTGCCTGTGGGAA | | |
| E6 gene_2 | ACAGTGGAACAAGAGTGTGGAAA | | |
| | GCCATTGGAAGA | | |
| gi\|9628574.E6\| | GACATGTGTATTTTGCAAAAATG | 120 | 75 |
| lcl\|HPV61_E6.1 | AATTAACAACAGAAGAATTGCTG | | |
| Human papillomavirus | GCGTTTGCACTAAAGGAGCTAAG | | |
| 61 (HPV61), | CATTGTGTGGAGACATAA | | |
| E6 gene_1 | CTGGCCATTTGGAGTATGCGCAC | | |
| | CATGCTTGGC | | |
| gi\|9628574.E6\| | ACGGTCATTAGCTGAACTATATA | 120 | 76 |
| lcl\|HPV61_E6.1 | TACGGTGCCATGCATGCAGCAAA | | |
| Human papillomavirus | CCGTTAAGTATACAGGAAAAGGA | | |
| 61 (HPV61), | GCATCAGGTACAGGCAT | | |
| E6 gene_2 | ACATCCACTTCCACTATATAGCT | | |
| | GGACAGTGGAC | | |
| 133448_5511683_gi\| | TTACCAAACGAGCAGGAAACGGA | 120 | 77 |
| 333071.E7\| | GGAGGAGCCTGACAACGAAAGGA | | |
| lcl\|HPV5_E7.1\| | TCTCTTACAAAGTTATAGCTCCG | | |
| Humanpapillomavirus | TGCGGTTGCAGGAACT | | |
| 5_1_2 | GTGAGGTCAAGCTTCGCATTTTT | | |
| | GTCCACGCCACA | | |
| 133448_5511684_gi\| | TAGACAGCTCAGAAGATGAGGTG | 120 | 78 |
| 60955.E7\| | GACGAAGTGGACGGACAAGATTC | | |
| lcl\|HPV6_E7.1\| | ACAACCTTTAAAACAACATTTCC | | |
| Humanpapillomavirus | AAATAGTGACCTGTTGC | | |
| 6_1_2 | TGTGGATGTGACAGCAACGTTCG | | |
| | ACTGGTTGTGC | | |
| 133448551 1685_gi\| | AAGACAGCTCAGAAGATGAGGTG | 120 | 79 |
| 333026.E7\| | GACAAGGTGGACAAACAAGACGC | | |
| lcl\|HPV11_E7.1\| | ACAACCTTTAACACAACATTACC | | |
| Humanpapillomavirus | AAATACTGACCTGTTG | | |
| _1_2 | CTGTGGATGTGACAGCAACGTCC | | |
| | GACTGGTTGTGG | | |
| 133448_5511686_gi\| | TTACCAAACGAGCAGGAAACGGA | 120 | 80 |
| 396910.E7\| | GGAGGAGTCAGATATCGACAGGA | | |
| lcl\|HPV12_E7.1\| | CTGTATTCAAAATCATTGCACCG | | |
| Humanpapillomavirus | TGTGGCTGCAGCTCCT | | |
| _1_2 | GTGAGGTCAACCTTCGTATTTTT | | |
| | GTCAACGCAACT | | |
| 133448_5511687_gi\| | ACAGCTCAGAGGAGGAGGATGAA | 120 | 81 |
| 333031.E7\| | ATAGATGGTCCAGCTGGACAAGC | | |
| lcl\|HPV16_E7.1\| | AGAACCGGACAGAGCCCATTACA | | |
| Humanpapillomavirus | ATATTGTAACCTTTTG | | |
| _1_2 | TTGCAAGTGTGACTCTACGCTTC | | |
| | GGTTGTGCGTAC | | |
| 133448_5511688_gi\| | TCAGAGGAAGAAAACGATGAAAT | 120 | 82 |
| 60975.E7\| | AGATGGAGTTAATCATCAACATT | | |
| lcl\|HPV18_E7.1\| | TACCAGCCCGACGAGCCGAACCA | | |
| Humanpapillomavirus | CAACGTCACACAATGTT | | |
| 1_1_2 | GTGTATGTGTTGTAAGTGTGAAG | | |
| | CCAGAATTGAG | | |
| 133448_5511689_gi\| | ATTTGACAGCTCAGATGAGGATG | 120 | 83 |
| 9627305.E7\| | AAACAGATAATATGCGTGACCAG | | |
| lcl\|HPV26_E7.1 | CAGGCCAGACAAGCTGGACAAGA | | |
| Humanpapillomavirus | AGTGTGTTACAGAATT | | |
| _1_2 | GAAGCACAATGTTGTATGTGTAA | | |
| | TAGTATAGTGCA | | |
| 133448_5511690_gi\| | ACAGCTCAGATGAGGAGGATGTC | 120 | 84 |
| 333048.E7\| | ATAGACAGTCCAGCTGGACAAGC | | |
| lcl\|HPV31_E7.1\| | AGAACCGGACACATCCAATTACA | | |
| Humanpapillomavirus | ATATCGTTACCTTTTGT | | |
| _1_2 | TGTCAGTGTAAGTCTACACTTCG | | |
| | TTTGTGTGTAC | | |
| 133448_5511691_gi\| | GACAGCTCAGATGAGGATGAAGG | 120 | 85 |
| 333049.E7\| | CTTGGACCGGCCAGATGGACAAG | | |
| lcl\|HPV33_E7.1\| | CACAACCAGCCACAGCTGATTAC | | |
| Humanpapillomavirus | TACATTGTAACCTGTT | | |
| _1_2 | GTCACACTTGTAACACCACAGTT | | |
| | CGTTTATGTGTC | | |
| 133448_5511692_gi\| | AGCTCAGAGGAGGAGGAAGATAC | 120 | 86 |
| 396997.E7\| | TATTGACGGTCCAGCTGGACAAG | | |
| lcl\|HPV35_E7.1\| | CAAAACCAGACACCTCCAATTAT | | |
| Humanpapillomavirus | AATATTGTAACGTCCTG | | |
| _1_2 | TTGTAAATGTGAGGCGACACTAC | | |
| | GTCTGTGTGTA | | |
| 133448_5511693_gi\| | TCCAGAGGATATTGAAGCATCAG | 120 | 87 |
| 1020234.E7\| | TGGTAGAGGAGGAGCCAGCATAC | | |
| lcl\|HPV38_E7.1 | ACCCCATACAAAATCATAGTTCT | | |
| Humanpapillomavirus | TTGTGGGGGTTGTGAAG | | |
| _1_2 | TAAGGCTAAAACTATACGTGTGG | | |
| | GCCACCGACGC | | |
| 133448_5511694_gi\| | GAGGATGAAATAGATGAACCCGA | 120 | 88 |
| 333245.E7\| | CCATGCAGTTAATCACCAACATC | | |
| lcl\|HPV39_E7.1\| | AACTACTAGCCAGACGGGATGAA | | |
| Humanpapillomavirus | CCACAGCGTCACACAA | | |
| _1_2 | TACAGTGTTCGTGTTGTAAGTGT | | |
| | AACAACACACTG | | |
| 133448_5511695_gi\| | CATGAACAGGACCAACTAGACAG | 120 | 89 |
| 397014.E7\| | CTTACACAGTAGAGAGCGTGAGC | | |
| lcl\|HPV40_E7.1\| | AACCCACGCAACAGGACCTGCAA | | |
| Humanpapillomavirus | GTAAATTTGCAATCAT | | |
| _1_2 | TTAAAGTAGTAACTCGGTGTGTA | | |
| | TTTTGTCAGTGT | | |
| 1334485511696_gi\| | TATGAACAATTGGACAGCTCAGA | 120 | 90 |
| 333211.E7\| | TGAAGATGACCAAGCCAAACAGG | | |
| lcl\|HPV42_E7.1\| | ACATACAGCGTTACAGAATACTG | | |
| Humanpapillomavirus | TGTGTGTGTACACAGTG | | |
| _1_2 | TTACAAGTCTGTTAAACTCGTTG | | |
| | TGCAGTGTACA | | |
| 133448_5511697_gi\| | AGTCAGAGGAGGAAAACGATGAA | 120 | 91 |
| 397022.E7\| | GCAGATGGAGTTAGTCATGCACA | | |
| lcl\|HPV45_E7.1\| | ACTACCAGCCCGACGAGCCGAAC | | |
| Humanpapillomavirus | CACAGCGTCACAAAA | | |
| _1_2 | TTTTGTGTGTATGTTGTAAGTGT | | |
| | GACGGCAGAATTG | | |
| 133448_5511698_gi\| | TCAGAGGAGGAGGATGAAGTAGA | 120 | 92 |
| 333087.E7\| | TAATATGCGTGACCAGCTACCAG | | |
| lcl\|HPV51_E7.1\| | AAAGACGGGCTGGACAGGCTACG | | |
| Humanpapillomavirus | TGTTACAGAATTGAAG | | |
| _1_2 | CTCCGTGTTGCAGGTGTTCAAGT | | |
| | GTAGTACAACTG | | |
| 133448_5511699_gi\| | AGCTCAGATGAGGAGGATACAGA | 120 | 93 |
| 397038.E7\| | TGGTGTGGACCGGCCAGATGGAC | | |
| lcl\|HPV52_E7.1\| | AAGCAGAACAAGCCACAAGCAAT | | |
| Humanpapillomavirus | TACTACATTGTGACAT | | |
| _1_2 | ATTGTCACAGTTGTGATAGCACA | | |
| | CTACGGCTATGC | | |
| 133448_5511700_gi\| | GATGAGGATGAGGATGAAGTAGA | 120 | 94 |
| 9627377.E7\| | CCATCTGCAGGAGCAGCCACAGC | | |
| lcl\|HPV53_E7.1 | AAGCTAGACGGGACGAACAACAT | | |
| Humanpapillomavirus | CCTTGTTACCTAATTG | | |
| _1_2 | AAACACAGTGTTGTAGGTGTGAG | | |
| | TCGTTGGTGCAG | | |
| 133448_5511701_gi\| | GAAGACTCTGATGCAGAAGATGA | 120 | 95 |
| 9628437.E7\| | GACAGCAGTAACACAACCTGACA | | |
| lcl\|HPV54_E7.1 | AACAAGCATTTAAGGTGTTAAGC | | |
| Humanpapillomavirus | CAGTGTGGAGGTGTGT | | |
| _1_2 | GTTGTAAGACAGTACGGCTATGT | | |
| | GTGTATAGCACG | | |
| 133448_5511702_gb\| | GACAGCTCAGAGGATGAGGTGGA | 120 | 96 |
| U31791.1\| | TGAACTAGCCACGCAGGCCACGC | | |
| HPU31791:530-823 | AAGACGTTACACAGCCTTACCAA | | |
| Humanpapillomavirus | ATAGTAACCACCTGT | | |
| _1_2 | GGTACATGTAACCGGAACGTTCG | | |
| | GCTGGTTGTGCAG | | |
| 133448_5511703_gi\| | GATGAGGATGAGGATGAAGTAGA | 120 | 97 |
| 397053.E7\| | CCATTTGCAGGAGCGGCCACAGC | | |
| lcl\|HPV56_E7.1\| | AAGCTAGACAAGCTAAACAACAT | | |
| Humanpapillomavirus | ACGTGTTACCTAATAC | | |
| _1_2 | ACGTACCTTGTTGTGAGTGTAAG | | |
| | TTTGTGGTGCAG | | |
| 133448_5511704_gi\| | ACAGCTCAGACGAGGATGAAATA | 120 | 98 |
| 222386.E7\| | GGCTTGGACGGGCCAGATGGACA | | |
| lcl\|HPV58_E7.1\| | AGCACAACCGGCCACAGCTAATT | | |
| Humanpapillomavirus | ACTACATTGTAACTTG | | |
| _1_2 | TTGTTACACTTGTGGCACCACGG | | |
| | TTCGTTTGTGTA | | |
| 133448_5511705_gi\| | GACTCCGAGAATGAAAAAGATGA | 120 | 99 |
| 557236.E7\| | ACCAGATGGAGTTAATCATCCTT | | |
| lcl\|HPV59_E7.1\| | TGCTACTAGCTAGACGAGCTGAA | | |
| Humanpapillomavirus | CCACAGCGTCACAACAT | | |
| _1_2 | TGTGTGTGTGTGTTGTAAGTGTA | | |
| | ATAATCAACTT | | |
| 133448_5511706_gi\| | TTAGACAGCTCAGAGTCAGAGGA | 120 | 100 |
| 9628574.E7\| | GGAGGATAGTGTGCGTGAGCAAC | | |
| lcl\|HPV61_E7.1 | TTGTTGAACAAGCACAGCAGGCC | | |
| Humanpapillomavirus | TACAGGGTGGTTACTA | | |
| _1_2 | CCTGTGGCATTTGTAAGTGTCCA | | |
| | GTTAGGCTGGTG | | |
| 133448_5511707_gi\| | AGACAGCTCAGAGGAAGAGGTGG | 120 | 101 |
| 39932599.E7\| | ATGATAGGGACAGTGCTGAGCAA | | |
| lcl\|HPV62_E7.1\| | CCTGTAGAACGGGCACAGCAGGC | | |
| Humanpapillomaviru | CCATAGGGTGGTTAC | | |
| _1_2 | TACGTGTGGTATTTGCAACTGTT | | |
| | TAGTTAGGTTGGT | | |
| 133448_5511708_gi\| | GATGAGGATGAGGATGAAATAGA | 120 | 102 |
| 1020290.E7\| | CCATTTGCTGGAGCGGCCACAGC | | |
| lcl\|HPV66_E7.1 | AAGCTAGACAAGCTGAACAACAT | | |
| Humanpapillomavirus | AAGTGTTACCTAATTCA | | |
| _1_2 | CGTACCTTGTTGTAAGTGTGAGT | | |
| | TGGTGGTGCAG | | |
| 133448_5511709_gi\| | AGCTCAGATGAGGAGGATGAAGT | 120 | 103 |
| 3228267.E7\| | AGGCATGGACCGGCCAGATGGAC | | |
| lcl\|HPV67_E7.1\| | AAGCAAAACCAGATACAACCAAT | | |
| Humanpapillomavirus | TACCATATTGTTACTG | | |
| _1_2 | TGTGTAACATCTGTGAGTGCACT | | |
| | TTGCGTTTGTGC | | |
| 133448_5511710_gi\| | ACGATGAAATAGATGAACCCGAC | 120 | 104 |
| 71726685.E7\| | CATGCAGTTAATCACCACCAACA | | |
| lcl\|HPV68_E7.1\| | TCTACTACTAGCCAGACGGGACG | | |
| Humanpapillomaviru | AACAACAGCGTCACAG | | |
| _1_2 | AATTCAGTGTCTGTGTTGTAAGT | | |
| | GTAACAAGGCAC | | |
| 133448_5511711_gi\| | ATTTGACAGCTCAGAGGAGGATG | 120 | 105 |
| 6970418.E7\| | AAACAGATAATGTGCGTAACCAG | | |
| lcl\|HPV69_E7.1\| | CAAGCCAGACAAGCTGAACAAGA | | |
| Humanpapillomavirus | AGCGTGTTATAGAATA | | |
| _1_2 | GAAGCGGAATGTTGTGTATGTAA | | |
| | TAGTATAGTGCA | | |
| 133448_5511712_gi\| | GACAATGAAACAGATGAACCCGA | 120 | 106 |
| 1173493.E7\| | CCATGTAGTTAATCACCAACAAC | | |
| lcl\|HPV70_E7.1\| | AACTACTAGCCAGACGGGAAGAA | | |
| Humanpapillomavirus | CCACAGCGTCACAAAA | | |
| _1_2 | TACAGTGTATGTGTTGTAAGTGT | | |
| | AATACTACACTG | | |
| 133448_5511713_gi\| | TTGCCAGCTCAGACGAGGAAGAT | 120 | 107 |
| 12084981.E7\| | AATAGGGTGGACGGTGAGCAACC | | |
| lcl\|HPV71_E7.1\| | CACAGAACCAGCACAGCAGGCAT | | |
| Humanpapillomaviru | ATAGGGTGGTTTCAT | | |
| _1_2 | ACTGTGGTAGGTGCTGTCGTGCA | | |
| | GTTAGGCTTGTGG | | |
| 133448_5511714_gi\| | CTCAGAGTCAGAGTCAGAGGATG | 120 | 108 |
| 1491683.E7\| | AGAGGGACGGTGTTGGTGTGCAG | | |
| lcl\|HPV72_E7.1 | GAGCAACTTGTAGAACAAGCACA | | |
| Humanpapillomavirus | GCAGGCCTACGGGGT | | |
| _1_2 | GGTTACTACCTGTGGCAGGTGCT | | |
| | ACCGTCCAGTTAG | | |
| 133448_5511715_gi\| | GACAACTCAGAGGATGAGGATGA | 120 | 109 |
| 1491692.E7\| | AACAGACAGCCATCTAGACAGAC | | |
| lcI\|HPV73_E7.1\| | AAGCTGAACGAGAGTGTTACAGA | | |
| Humanpapillomavirus | ATAGTTACTGACTGCA | | |
| _1_2 | CGAAGTGTCAGTGCACAGTATGC | | |
| | CTTGCCATTGAA | | |
| 133448_5511716_gi\| | ACAGCTCAGAGGAAGAGGTGGAT | 120 | 110 |
| 40804509.E7\| | GTGGATGATAGGGACAGTGGTGA | | |
| lcl\|HPV81_E7.1\| | GCAACTAGTAGAACAAGCACAGC | | |
| Humanpapillomaviru | AGGCCTATAGGGTGG | | |
| _1_2 | TTACTACGTGTGGTATTTGTAGG | | |
| | TGTTTAGTTAGGC | | |
| 133448_5511717_gi\| | CTCAGACGAGGAGGATGAAGTAG | 120 | 111 |
| 6970427.E7\| | ATAATATGCGTGACCAGCCAGCC | | |
| lcl\|HPV82_E7.1 | AGACAAGCTGGACAGGATACGTG | | |
| Humanpapillomavirus | TTACAGAATTAAAGTG | | |
| _1_2 | CACTGTTGCAGGTGTTCGAGTGT | | |
| | TGTACAGCTCGC | | |
| 133448_5511718_gi\| | GACAGCTCAGAGGAGGAGGATGA | 120 | 112 |
| 5059324.E7\| | TAGGGACTGTGGCGTGCGTGACC | | |
| lcl\|HPV83_E7.1 | AACTGGCAGAACAAGCAAAGCAG | | |
| Humanpapillomavirus | GCCTATAGGGTGTTA | | |
| _1_2 | ACAGTGTGTGGTATGTGTGGCCA | | |
| | GGCACTTAGACTA | | |
| 133448_5511719_gi\| | AGACAGCTCAGAGGAGGAGGATA | 120 | 113 |
| 12958167.E7\| | ATGGGGATTGCTTGCGTGACCAA | | |
| lcl\|HPV84_E7.1\| | CCTGCAAAACCAGCACAGCTGGC | | |
| Humanpapillomaviru | CTATAGGGTGGTAAC | | |
| _1_2 | AGAGTGTGGGTTGTGTAGCCGTC | | |
| | CAGTTAGGCTGGC | | |
| 133448_5511720_gi\| | ACAGAAGAGCAGTCAGAAAACCT | 120 | 114 |
| 50253426.E7\| | GTCAGAATCTACAGTAGCAGAGG | | |
| lcl\|HPV96_E7.1\| | TGGAGCCTCAGAGAACTCTGTAC | | |
| Humanpapillomaviru | AAGATTGTAGTACCGT | | |
| _1_2 | GTGGAGGCTGTGATTCCCGGCTG | | |
| | AAAATCTACGTT | | |

| HPV33_ | | | |
|---|---|---|---|
| SingleStrand_ | | | SEQ |
| SequenceName | Sequence | Length | ID#: |
| 129911_5270177_ | GTAAACTATAATGCCAAGTTTTAAA | 120 | 115 |
| HPV33REF_1_1 | AAAGTAGGGTGTAACCGAAAGCGGT | | |
| | TCAACCGAAAACGGTGCATATATAA | | |
| | AGCAAAC | | |
| | ATTTTGCAGTAAGGTACTGCACGAC | | |
| | TATGTTTCAAGAC | | |
| 129911_5270177_ | ACACTGAGGAAAAACCACGAACATT | 120 | 116 |
| HPV33REF_1_2 | GCATGATTTGTGCCAAGCATTGGAG | | |
| | ACAACTATACACAACATTGAACTAC | | |
| | AGTGCGT | | |
| | GGAATGCAAAAAACCTTTGCAACGA | | |
| | TCTGAGGTATATG | | |
| 129911_5270177_ | TGATTTTGCATTTGCAGATTTAACA | 120 | 117 |
| HPV33REF_1_3 | GTTGTATATAGAGAGGGAAATCCAT | | |
| | TTGGAATATGTAAACTGTGTTTGCG | | |
| | GTTCTTAT | | |
| | CTAAAATTAGTGAATATAGACATTA | | |
| | TAATTATTCTGT | | |
| 129911_5270177_ | GTATATGGAAATACATTAGAACAAA | 120 | 118 |
| HPV33REF_1_4 | CAGTTAAAAAACCTTTAAATGAAAT | | |
| | ATTAATTAGGTGTATTATATGTCAA | | |
| | AGACCTTTG | | |
| | TGTCCTCAAGAAAAAAAACGACATG | | |
| | TGGATTTAAAC | | |
| 129911_5270177_ | ACAAACGATTTCATAATATTTCGGG | 120 | 119 |
| HPV33REF_1_5 | TCGTTGGGCAGGGCGCTGTGCGGCG | | |
| | TGTTGGAGGTCCCGACGTAGAGAAA | | |
| | CTGCA | | |
| | CTGTGACGTGTAAAAACGCCATGAG | | |
| | AGGACACAAGCCAAC | | |
| 1299115270177_ | ACGTTAAAGGAATATGTTTTAGATT | 120 | 120 |
| HPV33REF_1_6 | TATATCCTGAACCAACTGACCTATA | | |
| | CTGCTATGAGCAATTAAGTGACAGC | | |
| | TCAGATGA | | |
| | GGATGAAGGCTTGGACCGGCCAGAT | | |
| | GGACAAGCACAA | | |
| 129911_5270177_ | AACCAGCCACAGCTGATTACTACAT | 120 | 121 |
| HPV33REF_1_7 | TGTAACCTGTTGTCACACTTGTAAC | | |
| | ACCACAGTTCGTTTATGTGTCAACA | | |
| | GTACAGC | | |
| | AAGTGACCTACGAACCATACAGCAA | | |
| | CTACTTATGGGCA | | |
| 129911_5270177_ | CACAGTGAATATTGTGTGCCCTACC | 120 | 122 |
| HPV33REF_1_8 | TGTGCACAACAATAAACATCATCTA | | |
| | CAATGGCCGATCCTGAAGGTACAAA | | |
| | TGGGGC | | |
| | TGGGATGGGGTGTACTGGTTGGTTT | | |
| | GAGGTAGAAGCAGT | | |
| 129911_5270177_ | GTCATAGAGAGAAGAACAGGAGATA | 120 | 123 |
| HPV33REF_1_9 | ATATTTCAGAAGATGAGGATGAAAC | | |
| | AGCAGATGACAGTGGCACGGATTTA | | |
| | CTAGAG | | |
| | TTTATAGATGATTCTATGGAAAATA | | |
| | GTATACAGGCAGAC | | |
| 129911_5270177_ | ACACAGAGGCAGCCCGGGCATTGTT | 120 | 124 |
| HPV33REF_1_10 | TAATATACAGGAAGGGGAGGATGAT | | |
| | TTAAATGCTGTGTGTGCACTAAAAC | | |
| | GAAAGT | | |
| | TTGCCGCATGTTCACAAAGTGCTGC | | |
| | GGAGGACGTTGTTG | | |
| 129911_5270177_ | TGATCGTGCTGCAAACCCGTGTAGA | 120 | 125 |
| HPV33REF_1_11 | ACGTCTATTAATAAAAATAAAGAAT | | |
| | GCACATACAGAAAACGAAAAATAGA | | |
| | TGAGCTA | | |
| | GAAGACAGCGGATATGGCAATACTG | | |
| | AAGTGGAAACTCA | | |
| 129911_5270177_ | CAGCAGATGGTACAACAGGTAGAAA | 120 | 126 |
| HPV33REF_1_12 | GTCAAAATGGCGACACAAACTTAAA | | |
| | TGACTTAGAATCTAGTGGGGTGGGG | | |
| | GATGAT | | |
| | TCAGAAGTAAGCTGTGAGACAAATG | | |
| | TAGATAGCTGTGAA | | |
| 129911_5270177_ | AAAATGTTACGTTGCAGGAAATTAG | 120 | 127 |
| HPV33REF_1_13 | TAATGTTCTACATAGTAGTAATACA | | |
| | AAAGCAAATATATTATATAAATTTA | | |
| | AAGAGGCCT | | |
| | ATGGAATAAGTTTTATGGAATTAGT | | |
| | AAGACCATTTA | | |
| 129911_5270177_ | TAAAAGTGATAAAACAAGCTGTACA | 120 | 128 |
| HPV33REF_1_14 | GATTGGTGTATAACAGGATATGGAA | | |
| | TTAGTCCATCAGTAGCAGAAAGTTT | | |
| | AAAAGTAT | | |
| | TAATTAAACAGCATAGTTTGTATAC | | |
| | TCATTTACAATG | | |
| 129911_5270177_ | TGTTTAACTTGCGATAGAGGAATAA | 120 | 129 |
| HPV33REF_1_15 | TAATATTATTGTTAATTAGATTTAG | | |
| | GTGTAGCAAAAACAGGTTAACAGTA | | |
| | GCAAAACT | | |
| | AATGAGTAATTTATTATCAATACCT | | |
| | GAAACATGTATG | | |
| 129911_5270177_ | TGGTTATAGAGCCACCAAAATTACG | 120 | 130 |
| HPV33REF_1_16 | GAGCCAAACATGTGCATTGTATTGG | | |
| | TTTAGAACAGCAATGTCAAACATTA | | |
| | GTGATGT | | |
| | ACAAGGTACAACACCTGAATGGATA | | |
| | GATAGACTAACTG | | |
| 129911_5270177_ | TGTTTTACAACATAGCTTTAATGAT | 120 | 131 |
| HPV33REF_1_17 | AATATATTTGATTTAAGTGAAATGG | | |
| | TACAGTGGGCATATGATAACGAGTT | | |
| | AACGGACG | | |
| | ATAGTGACATTGCATATTATTATGC | | |
| | ACAACTTGCAGA | | |
| 129911_5270177_ | GATTCAAATAGTAATGUTGUTGCAT | 120 | 132 |
| HPV33REF_1_18 | TTTTAAAAAGTAACTCACAAGCAAA | | |
| | AATAGTAAAGGACTGTGGAATAATG | | |
| | TGTAGACA | | |
| | TTATAAAAAAGCAGAAAAACGTAAA | | |
| | ATGTCAATAGGA | | |
| 129911_5270177_ | GACAATGGATACAAAGTAGATGTGA | 120 | 133 |
| HPV33REF_1_19 | AAAAACAAATGATGGAGGAAATTGG | | |
| | AGACCAATAGTACAGTTGTTAAGAT | | |
| | ATCAAAA | | |
| | GATTGAATTTACAGCATTTTTAGGT | | |
| | GCATTTAAAAAGT | | |
| 129911_5270177_ | GTTTTTAAAAGGTATACCAAAAAAA | 120 | 134 |
| HPV33REF_1_20 | AGCTGTATGCTAATTTGTGGACCAG | | |
| | CAAATACAGGAAAGTcATATTTTGG | | |
| | AATGAGTT | | |
| | TAATACAGTTTTTAAAAGGGTGTGT | | |
| | TATATCATGTGT | | |
| 129911_5270177_ | GTAAATTCTAAAAGTCACTTTTGGT | 120 | 135 |
| HPV33REF_1_21 | TGCAGCCATTATCAGATGCAAAAAT | | |
| | AGGAATGATAGATGATGTAACGCCA | | |
| | ATAAGTTG | | |
| | GACATATATAGATGATTACATGAGA | | |
| | AATGCGTTAGAT | | |
| 129911_5270177_ | ATGGAAATGAAATTTCAATAGATGT | 120 | 136 |
| HPV33REF_1_22 | GAAACATAGGGCATTAGTGCAATTA | | |
| | AAATGTCCACCACTGCTTCTTACCT | | |
| | CAAATACA | | |
| | AATGCAGGCACAGACTCTAGATGGC | | |
| | CATATTTACATA | | |
| 129911_5270177_ | TAGTAGATTAACAGTATTTGAATTT | 120 | 137 |
| HPV33REF_1_23 | AAAAATCCATTCCCATTTGATGAAA | | |
| | ATGGTAACCCAGTGTATGCAATAAA | | |
| | TGATGAAAA | | |
| | TTGGAAATCCTTTTTCTCAAGGACG | | |
| | TGGTGCAAATT | | |
| 129911_5270177_ | TTAGATTTAATAGAGGAAGAGGACA | 120 | 138 |
| HPV33REF_1_24 | AGGAAAACCATGGAGGAAATATCAG | | |
| | CACGTTTAAATGCAGTGCAGGAGAA | | |
| | AATACTA | | |
| | GATCTTTACGAAGCTGATAAAACTG | | |
| | ATTTACCATCACA | | |
| 129911_5270177_ | CAAATTGAACATTGGAAACTGATAC | 120 | 139 |
| HPV33REF_1_25 | GCATGGAGTGTGCTTTATTGTATAC | | |
| | AGCCAAACAAATGGGATTTTCACAT | | |
| | TTATGCCA | | |
| | CCAGGTGGTGCCTTCTTTGTTAGCA | | |
| | TCAAAGACCAAA | | |
| 129911_5270177_ | AAGCATTTCAAGTAATTGAACTACA | 120 | 140 |
| HPV33REF_1_26 | AATGGCATTAGAGACATTAAGTAAA | | |
| | TCACAGTATAGTACAAGCCAATGGA | | |
| | CATTGCAA | | |
| | CAAACAAGCTTAGAGGTGTGGCTTT | | |
| | GTGAACCACCAA | | |
| 129911_5270177_ | AAAATGTTTTAAAAAACAAGGAGAA | 120 | 141 |
| HPV33REF_1_27 | ACAGTAACTGTGCAATATGACAATG | | |
| | ACAAAAAAAATACAATGGATTATAC | | |
| | AAACTGGG | | |
| | GTGAAATATATATTATAGAGGAAGA | | |
| | TACATGTACTAT | | |
| 129911_5270177_ | ATGGTTACAGGGAAAGTAGATTATA | 120 | 142 |
| HPV33REF_1_28 | TAGGTATGTATTATATACATAACTG | | |
| | TGAAAAGGTATATTTTAAATATTTT | | |
| | AAAGAGGAT | | |
| | GCTGCAAAGTATTCTAAAACACAAA | | |
| | TGTGGGAAGTA | | |
| 129911_5270177_ | TACATGTGGGTGGTCAGGTAATTGT | 120 | 143 |
| HPV33REF_1_29 | TTGTCCTACGTCTATATCTAGCAAC | | |
| | CAAATATCCACTACTGAAACTGCTG | | |
| | ACATACA | | |
| | GACAGACAACGATAACCGACCACCA | | |
| | CAAGCAGCGGCCA | | |
| 129911_5270177_ | CAAACGACGACGACCTGCAGACACC | 120 | 144 |
| HPV33REF_1_30 | ACAGACACCGCCCAGCCCCTTACAA | | |
| | AGCTGTTCTGTGCAGACCCCGCCTT | | |
| | GGACA | | |
| | ATAGAACAGCACGTACTGCAACTAA | | |
| | CTGCACAAACAAGCA | | |
| 129911_5270177_ | CAGCGGACTGTGTGTAGTTCTAACG | 120 | 145 |
| HPV33REF_1_31 | TTGCACCTATAGTGCATTTAAAAGG | | |
| | TGAATCAAATAGTTTAAAATGTTTA | | |
| | AGATACAG | | |
| | ATTAAAACCTTATAAAGAGTTGTAT | | |
| | AGTTCTATGTCA | | |
| 129911_5270177_ | CATCCACCTGGCATTGGACCAGTGA | 120 | 146 |
| HPV33REF_1_32 | CAACAAAAATAGTAAAAATGGAATT | | |
| | GTAACTGTAACATTTGTAACTGAAC | | |
| | AGCAACA | | |
| | ACAAATGTTTTTAGGTACCGTAAAA | | |
| | ATACCACCTACTG | | |
| 129911_5270177_ | TGTGCAAATAAGTACTGGATTTATG | 113 | 147 |
| HPV33REF_1_33 | ACATTATAAGTGTACATCACAAGCC | | |
| | AATATGTGCTGCTAATTGTATATAA | | |
| | CCATGATA | | |
| | TTTGTTTTTGtattatgttttatat | | |
| | tgttt | | |
| 129911_5270178_ | tatgcttatccttattattaCGTCC | 120 | 148 |
| HPV33REF_2_1 | TTTAATACTTTCCATTTCTACCTAT | | |
| | GCTTGGTTGCTGGTGTTGGTATTGC | | |
| | TGCTTTGGGTGTTTGT | | |
| | GGGATCTCCTTTAAAAATTTTTTTT | | |
| | TGCT | | |
| 129911_5270178_ | CTATTTGTTGTTTTTATATTTACCA | 120 | 149 |
| HPV33REF_2_2 | ATGATGTGTATTAATTTTCATGCAC | | |
| | AGCATATGACACAACAAGAGTAATG | | |
| | TATATACAT | | |
| | GTATATATTGTTTGTATATATGTGC | | |
| | ACATGGTGGTG | | |
| 129911_5270178_ | TGTTTTAACATTGTTGTTGTTATTT | 120 | 150 |
| HPV33REF_2_3 | TAGTTTTTTTTTTTTTGTATTACTA | | |
| | ATAAATACCTTTATATTTTAGCAGT | | |
| | GTATTATTATGA | | |
| | GACACAAACGATCTACAAGGCGCAA | | |
| | GCGTGCAT | | |
| 129911_5270178_ | ATCTGCAACACAACTATACCAAACA | 120 | 151 |
| HPV33REF_2_4 | TGCAAGGCCACAGGCACCTGCCCAC | | |
| | CCGATGTTATTCCTAAAGTGGAAGG | | |
| | AAGTAC | | |
| | CATAGCAGATCAAATTCTTAAATAT | | |
| | GGCAGTTTAGGGGT | | |
| 129911_5270178_ | GTTTTTTTTGGTGGTTTAGGTATTG | 120 | 152 |
| HPV33REF_2_5 | GCACAGGCTCTGGTTCAGGTGGAAG | | |
| | GACTGGCTATGTACCTATTGGTACT | | |
| | GACCCAC | | |
| | CTACAGCTGCAATCCCCTTGCAGCC | | |
| | TATACGTCCTCCG | | |
| 129911_5270178_ | CGGTTACTGTAGACACTGTTGGACC | 120 | 153 |
| HPV33REF_2_6 | TTTAGACTCGTCTATAGTGTCATTA | | |
| | ATAGAAGAAACAAGTTTTATAGAGG | | |
| | CAGGTGC | | |
| | ACCAGCCCCATCTATTCCTACACCA | | |
| | TCAGGTTTTGATG | | |
| 129911_5270178_ | TGTTACTACATCTGCAGATACTACA | 120 | 154 |
| HPV33REF_2_7 | CCTGCAATTATTAATGTTTCATCTG | | |
| | TTGGGGAGTCATCTATTCAAACTAT | | |
| | TTCTACACA | | |
| | TTTAAATCCCACATTTACTGAACCA | | |
| | TCTGTACTACA | | |
| 129911_5270178_ | CACCCTCCAGCGCCTGCAGAAGCCT | 120 | 155 |
| HPV33REF_2_8 | CTGGACATTTTATATTTTCTTCCCC | | |
| | TACTGTTAGCACACAAAGTTATGAA | | |
| | AACATAC | | |
| | CAATGGATACCTTTGTTGTTTCCAC | | |
| | AGACAGTAGTAAT | | |
| 129911_5270178_ | ATGTAACATCAAGCACGCCCATTCC | 120 | 156 |
| HPV33REF_2_9 | AGGGTCTCGCCCTGTGGCACGCCTT | | |
| | GGTTTATATAGTCGCAATACCCAAC | | |
| | AGGTTA | | |
| | AGGTTGTTGACCCTGCTTTTTTAAC | | |
| | ATCGCCTCATAAAC | | |
| 129911_5270178_ | ACTTATAACATATGATAATCCTGCA | 120 | 157 |
| HPV33REF_2_10 | TTTGAAAGCTTTGACCCTGAAGACA | | |
| | CATTACAATTTCAACATAGTGATAT | | |
| | ATCACCTG | | |
| | CTCCTGATCCTGACTTTCTAGATAT | | |
| | TATTGCATTACA | | |
| 129911_5270178_ | CATAGGCCTGCTATTACATCTCGTA | 120 | 158 |
| HPV33REF_2_11 | GACATACTGTGCGTTTTAGTAGAGT | | |
| | AGGTCAAAAAGCCACACTTAAAACT | | |
| | CGCAGTG | | |
| | GTAAACAAATTGGAGCTAGAATACA | | |
| | TTATTATCAGGAT | | |
| 129911_5270178_ | ATTTAAGTCCTATTGTGCCTTTAGA | 120 | 159 |
| HPV33REF_2_12 | CCACACCGTGCCAAATGAACAATAT | | |
| | GAATTACAGCCTTTACATGATACTT | | |
| | CTACATCG | | |
| | TCTTATAGTATTAATGATGGTTTGT | | |
| | ATGATGTTTATG | | |
| 129911_5270178_ | TGCTGACGATGTGGATAATGTACAC | 120 | 160 |
| HPV33REF_2_13 | ACCCCAATGCAACACTCATACAGTA | | |
| | CGTTTGCAACAACACGTACCAGCAA | | |
| | TGTGTCT | | |
| | ATACCTTTAAATACAGGATTTGATA | | |
| | CTCCTGTTATGTC | | |
| 129911_5270178_ | TCTGGCCCTGATATACCTTCCCCTT | 120 | 161 |
| HPV33REF_2_14 | TATTTCCCACATCTAGCCCATTTGT | | |
| | TCCTATTTCGCCTTTTTTTCCTTTT | | |
| | GACACCATT | | |
| | GTTGTAGACGGTGCTGACTTTGTTT | | |
| | TACATCCTAGT | | |
| 129911_5270178_ | GTTATTTTATTTTACGTCGCAGGCG | 120 | 162 |
| HPV33REF_2_15 | TAAACGTTTTCCATATTTTTTTACA | | |
| | GATGTCCGTGTGGCGGCCTAGTGAG | | |
| | GCCACAG | | |
| | TGTACCTGCCTCCTGTACCTGTATC | | |
| | TAAAGTTGTCAGC | | |
| 129911_5270178_ | GCACTGATGAATATGTGTCTCGCAC | 120 | 163 |
| HPV33REF_2_16 | AAGCATTTATTATTATGCTGGTAGT | | |
| | TCCAGACTTCTTGCTGTTGGCCATC | | |
| | CATATTTT | | |
| | TCTATTAAAAATCCTACTAACGCTA | | |
| | AAAAATTATTGG | | |
| 129911_5270178_ | GGTACCCAAAGTATCAGGCTTGCAA | 120 | 164 |
| HPV33REF_2_17 | TATAGGGTTTTTAGGGTCCGTTTAC | | |
| | CAGATCCTAATAAATTTGGATTTCC | | |
| | TGACACC | | |
| | TCCTTTTATAACCCTGATACACAAC | | |
| | GATTAGTATGGGC | | |
| 129911_5270178_ | GCATGTGTAGGCCTTGAAATAGGTA | 120 | 165 |
| HPV33REF_2_18 | GAGGGCAGCCATTAGGCGTTGGCAT | | |
| | AAGTGGTCATCCTTTATTAAACAAA | | |
| | TTTGATG | | |
| | ACACTGAAACCGGTAACAAGTATCC | | |
| | TGGACAACCGGGT | | |
| 129911_5270178_ | GTGCTGATAATAGGGAATGTTTATC | 120 | 166 |
| HPV33REF_2_19 | CATGGATTATAAACAAACACAGTTA | | |
| | TGTTTACTTGGATGTAAGCCTCCAA | | |
| | CAGGGGA | | |
| | ACATTGGGGTAAAGGTGTTGCTTGT | | |
| | ACTAATGCAGCAC | | |
| 129911_5270178_ | ACCTGCCAATGATTGTCCACCTTTA | 120 | 167 |
| HPV33REF_2_20 | GAACTTATAAATACTATTATTGAGG | | |
| | ATGGTGATATGGTGGACACAGGATT | | |
| | TGGTTGC | | |
| | ATGGATTTTAAAACATTGCAGGCTA | | |
| | ATAAAAGTGATGT | | |
| 129911_5270178_ | GTTCCTATTGATATTTGTGGCAGTA | 120 | 168 |
| HPV33REF_2_21 | CATGCAAATATCCAGATTATTTAAA | | |
| | AATGACTAGTGAGCCTTATGGTGAT | | |
| | AGTTTATTT | | |
| | TTCTTTCTTCGACGTGAACAAATGT | | |
| | TTGTAAGACAC | | |
| 129911_5270178_ | ACTTTTTTAATAGGGCTGGTACATT | 120 | 169 |
| HPV33REF_2_22 | AGGAGAGGCTGTTCCCGATGACCTG | | |
| | TACATTAAAGGTTCAGGAACTACTG | | |
| | CCTCTAT | | |
| | TCAAAGCAGTGCTTTTTTTCCCACT | | |
| | CCTAGTGGATCAA | | |
| 129911_5270178_ | AATGGTTACTTCCGAATCTCAGTTA | 120 | 170 |
| HPV33REF_2_23 | TTTAATAAGCCATATTGGCTACAAC | | |
| | GTGCACAAGGTCATAATAATGGTAT | | |
| | TTGTTGGG | | |
| | GCAATCAGGTATTTGTTACTGTGGT | | |
| | AGATACCACTCG | | |
| 129911_5270178_ | CGCAGTACTAATATGACTTTATGCA | 120 | 171 |
| HPV33REF_2_24 | CACAAGTAACTAGTGACAGTACATA | | |
| | TAAAAATGAAAATTTTAAAGAATAT | | |
| | ATAAGACAT | | |
| | GTTGAAGAATATGATCTACAGTTTG | | |
| | TTTTTCAACTA | | |
| 129911_5270178_ | TATGCAAAGTTACCTTAACTGCAGA | 120 | 172 |
| HPV33REF_2_25 | AGTTATGACATATATTCATGCTATG | | |
| | AATCCAGATATTTTAGAAGATTGGC | | |
| | AATTTGGTT | | |
| | TAACACCTCCTCCATCTGCTAGTTT | | |
| | ACAGGATACCT | | |
| 129911_5270178_ | CTATAGGTTTGTTACCTCTCAGGCT | 120 | 173 |
| HPV33REF_2_26 | ATTACGTGTCAAAAAACAGTACCTC | | |
| | CAAAGGAAAAGGAAGACCCCTTAGG | | |
| | TAAATAT | | |
| | ACATTTTGGGAAGTGGATTTAAAGG | | |
| | AAAAATTTTCAGC | | |
| 129911_5270178_ | GCAGATTTAGATCAGTTTCCTTTGG | 120 | 174 |
| HPV33REF_2_27 | GACGCAAGTTTTTATTACAGGCAGG | | |
| | TCTTAAAGCAAAACCTAAACTTAAA | | |
| | CGTGCAG | | |
| | CCCCCACATCCACCCGCACATCGTC | | |
| | TGCAAAACGCAAA | | |
| 129911_5270178_ | AAAAGGTTAAAAAATAACACTTTGT | 120 | 175 |
| HPV33REF_2_28 | GTAATTGTGTTATGTTGTTGTTTTG | | |
| | TTCTGTCTATGTACTTTGTGTTGTT | | |
| | GTGTTGTGTT | | |
| | GTTGTTTGTTTTTTGTGTATGTGTT | | |
| | ACAATGTATG | | |
| 129911_5270178_ | TGTTATGTTGTATGTTACTGTGTTT | 120 | 176 |
| HPV33REF_2_29 | GTTTTATGTGTACTTGTTTGTGTGC | | |
| | ATGTTCTATGTACTTGTCAGTTTCC | | |
| | TGTTTGTGTA | | |
| | TATGTTAATAAAACATTGTGTGTAT | | |
| | TTGTTAAACT | | |
| 129911_5270178_ | CTATTTGTATGTATGTTATGTATAT | 120 | 177 |
| HPV33REF_2_30 | GGGTGTACCTATATGAGTAAGGAGT | | |
| | TGTATTGCTTGCCCTACCCTGCATT | | |
| | GCAATGTA | | |
| | CCTACCTTTATTTCCCTATATTTGT | | |
| | AGTACCTACATG | | |
| 129911_5270178_ | TGTTTAGTATTGCTTTACCTTTTGA | 120 | 178 |
| HPV33REF_2_31 | CATACTAGTGTCCATATTGTACAAT | | |
| | TTCCTCCATTTTGTATGCCTAACCG | | |
| | TTTTCGGTT | | |
| | ACTTGGCATACATACCCTATGACAT | | |
| | TGGCAGAACAG | | |
| 129911_5270178_ | AGTTAATCCTTTTCTTTCCTGCACT | 120 | 179 |
| HPV33REF_2_32 | GTGTTTGTCTGTACTTGCTGCATTG | | |
| | GCATACATACCCTATGACATTGGCA | | |
| | GAACAGTT | | |
| | AATCCTTTTCTTTCCTGCACTGTGT | | |
| | TTGTCTGTACTT | | |
| 129911_5270178_ | TTGCTGCATTGACTCATATATACAT | 120 | 180 |
| HPV33REF_2_33 | GCAGTGCAATTGCAAAATACTTAAT | | |
| | TGTACTAATAGTTTACACATGCTTT | | |
| | TAGGCACAT | | |
| | ATTTTTACTTTACTTTCAAACCTTA | | |
| | AGTGCAGTTTT | | |
| 129911_5270178_ | TTGGCTTACACAATTGCTTTGTATG | 120 | 181 |
| HPV33REF_2_34 | CCAAACTATGCCTTGTAAAAGTGAG | | |
| | TCACTACCTGTTTATTACCAGGTGT | | |
| | GGACTAAC | | |
| | CGTTTTAGGTCATATTGGTCATTTA | | |
| | TAATCTTTTATA | | |

| HPV45_SingleStrand_ | | | SEQ |
|---|---|---|---|
| SequenceName | Sequence | Length | ID#: |
| 129911_5270179_ | AATACTTTTAACAATTATACTACAT | 120 | 182 |
| HPV45REF_1_1 | AAAAAAGGGTGTAACCGAAAACGGT | | |
| | TGCAACCAAAAACGGTGCATATAAA | | |
| | AGCTTTGTGGAAAAGTGCA | | |
| | TTACAGGATGGCGCGCTTTGACGAT | | |
| | C | | |
| 129911_5270179_ | CAAAGCAACGACCCTACAAGCTACC | 120 | 183 |
| HPV45REF12 | AGATTTGTGCACAGAATTGAATACA | | |
| | TCACTACAAGACGTATCTATTGCCT | | |
| | GTGTATATTGCAAAGCAAC | | |
| | ATTGGAACGCACAGAGGTATATCAA | | |
| | T | | |
| 129911_5270179_ | TTGCTTTTAAAGATTTATGTATAGT | 120 | 184 |
| HPV45REF_1_3 | GTATAGAGACTGTATAGCATATGCT | | |
| | GCATGCCATAAATGTATAGACTTTT | | |
| | ATTCCAGAATTAGAGAATTAA | | |
| | GATATTATTCAAACTCTGTATATG | | |
| 129911_5270179_ | GAGAGACACTGGAAAAAATAACTAA | 120 | 185 |
| HPV45REF_1_4 | TACAGAGTTGTATAATTTGTTAATA | | |
| | AGGTGCCTGCGGTGCCAGAAACCAT | | |
| | TGAACCCAGCAGAAAAAC | | |
| | GTAGACACCTTAAGGACAAACGAAG | | |
| | AT | | |
| 129911_5270179_ | TTCACAGCATAGCTGGACAGTACCG | 120 | 186 |
| HPV45REF_1_5 | AGGGCAGTGTAATACATGTTGTGAC | | |
| | CAGGCACGGCAAGAAAGACTTCGCA | | |
| | GACGTAGGGAAACACAA | | |
| | GTATAGCAATAAGTATGCATGGACC | | |
| | CCG | | |
| 129911_5270179_ | GGAAACACTGCAAGAAATTGTATTG | 120 | 187 |
| HPV45REF_1_6 | CATTTGGAACCTCAGAATGAATTAG | | |
| | ATCCTGTTGACCTGTTGTGTTACGA | | |
| | GCAATTAAGCGAGTCAGAG | | |
| | GAGGAAAACGATGAAGCAGATGGAG | | |
| | T | | |
| 129911_5270179_ | TAGTCATGCACAACTACCAGCCCGA | 120 | 188 |
| HPV45REF_1_7 | CGAGCCGAACCACAGCGTCACAAAA | | |
| | TTTTGTGTGTATGTTGTAAGTGTGA | | |
| | CGGCAGAATTGAGCTTAC | | |
| | AGTAGAGAGCTCGGCAGAGGACCTT | | |
| | AG | | |
| 129911_5270179_ | AACACTACAGCAGCTGTTTTTGAGC | 120 | 189 |
| HPV45REF_1_8 | ACCTTGTCCTTTGTGTGTCCGTGGT | | |
| | GTGCAACTAACCAATAATCTACAAT | | |
| | GGCGGATCCAGAAGGTAC | | |
| | CGACGGGGAGGGAACGGGGTGTAAT | | |
| | GG | | |
| 129911_5270179_ | CTGGTTCTTTGTAGAAACAATTGTA | 120 | 190 |
| HPV45REF_1_9 | GAGAAAAAAACAGGGGATGTAATAT | | |
| | CAGATGATGAGGATGAAACTGCAAC | | |
| | AGATACAGGGTCGGATAT | | |
| | GGTAGATTTTATTGACACACAATTA | | |
| | TC | | |
| 129911_5270179_ | CATTTGTGAACAGGCAGAGCAAGAG | 120 | 191 |
| HPV45REF_1_10 | ACAGCACAGGCATTGTTCCATGCGC | | |
| | AGGAAGTTCAGAATGATGCACAGGT | | |
| | GTTGCATCTTTTAAAACG | | |
| | AAAGTTTGCAGGAGGCAGCAAGGAA | | |
| | AA | | |
| 129911_5270179_ | CAGTCCATTAGGGGAGCAGCTAAGT | 120 | 192 |
| HPV45REF_1_11 | GTGGATACGGATCTAAGTCCACGGT | | |
| | TACAAGAAATTTCATTAAATAGTGG | | |
| | GCACAAAAAAGCAAAACG | | |
| | ACGGTTGTTTACAATATCAGATAGT | | |
| | GG | | |
| 129911_5270179_ | CTATGGCTGTTCTGAAGTGGAAGCT | 120 | 193 |
| HPV45REF_1_12 | GCAGAGACTCAGGTAACTGTAAACA | | |
| | CTAATGCGGAAAATGGCGGCAGTGT | | |
| | ACATAGTACACAAAGTA | | |
| | GTGGTGGGGATAGTAGTGACAATGC | | |
| | AGA | | |
| 129911_5270179_ | AAATGTAGATCCGCATTGCAGTATT | 120 | 194 |
| HPV45REF_1_13 | ACAGAACTAAAGGAGCTATTACAAG | | |
| | CAAGTAACAAAAAGGCTGCAATGCT | | |
| | GGCAGTATTTAAAGACATA | | |
| | TATGGGCTGTCATTTACGGATTTGG | | |
| | T | | |
| 129911_5270179_ | TAGAAATTTTAAAAGTGATAAAACA | 120 | 195 |
| HPV45REF_1_14 | ACATGTACAGATTGGGTAATGGCTA | | |
| | TATTTGGAGTTAATCCAACGGTAGC | | |
| | AGAAGGCTTTAAAACATTAA | | |
| | TTAAACCAGCAACGTTATACGCCCA | | |
| 129911_5270179_ | TATCCAATGTTTAGATTGTAAATGG | 120 | 196 |
| HPV45REF_1_15 | GGAGTATTAATATTAGCTTTATTAA | | |
| | GATATAAATGTGGCAAAAATAGACT | | |
| | AACTGTTGCAAAAGGCTTAA | | |
| | GCACATTGTTGCACGTACCTGAAAC | | |
| 129911_5270179_ | ATGTATGTTAATTGAACCACCAAAA | 120 | 197 |
| HPV45REF_1_16 | TTGCGAAGTAGTGTTGCAGCATTAT | | |
| | ACTGGTATAGAACAGGTATATCCAA | | |
| | TATTAGTGAAGTAAGTGGA | | |
| | GACACACCTGAGTGGATACAAAGAC | | |
| | T | | |
| 129911_5270179_ | GACAATTATTCAACATGGTATTGAC | 120 | 198 |
| HPV45REF_1_17 | GATAGTAATTTTGATTTGTCAGACA | | |
| | TGGTGCAATGGGCATTTGATAATGA | | |
| | CCTTACAGATGAAAGTGATA | | |
| | TGGCATTTCAATATGCCCAATTAGC | | |
| 129911_5270179_ | AGACTGCAACAGTAATGCAGCTGCA | 120 | 199 |
| HPV45REF_1_18 | TTTTTAAAAAGTAACTGCCAAGCCA | | |
| | AATATTTAAAAGATTGTGCTGTAAT | | |
| | GTGTAGACATTATAAAAGAG | | |
| | CACAAAAACGCCAAATGAATATGTC | | |
| 129911_5270179_ | TCAATGGATTAAATATAGATGTTCC | 120 | 200 |
| HPV45REF_1_19 | AAAATAGATGAAGGTGGGGATTGGA | | |
| | GACCCATAGTACAATTCCTAAGATA | | |
| | TCAGGGAGTAGAATTTATT | | |
| | AGCTTTTTAAGGGCACTAAAGGAAT | | |
| | T | | |
| 129911_5270179_ | TCTTAAAGGAACACCAAAAAAAAAT | 120 | 201 |
| HPV45REF_1_20 | TGTATACTGTTATATGGACCTGCAA | | |
| | ATACAGGAAAATCGTATTTTGGAAT | | |
| | GAGTTTTATACATTTCCTACA | | |
| | AGGTGCAATAATATCATTTGTAAA | | |
| 129911_5270179_ | TTCAAACAGCCATTTTTGGTTAGAA | 120 | 202 |
| HPV45REF_1_21 | CCGTTAGCAGATACTAAGGTAGCCA | | |
| | TGTTGGATGATGCCACACACACGTG | | |
| | TTGGACATATTTTGATAAT | | |
| | TATATGAGAAATGCATTAGATGGTA | | |
| | A | | |
| 129911_5270179_ | TCCTATAAGTATAGACAGAAAGCAT | 120 | 203 |
| HPV45REF_1_22 | AAACCATTATTACAGCTAAAATGTC | | |
| | CTCCAATCCTATTAACATCCAATAT | | |
| | TGATCCAGCAAAAGATAATA | | |
| | AATGGCCATATTTAGAAAGTAGGGT | | |
| 129911_5270179_ | GACGGTATTTACATTTCCACATGCA | 120 | 204 |
| HPV45REF_1_23 | TTTCCATTTGATAAAAATGGTAATC | | |
| | CAGTATATGAAATAAATGATAAAAA | | |
| | TTGGAAATGTTTTTTTGAAAG | | |
| | GACATGGTCCAGATTAGATTTGCA | | |
| 129911_5270179_ | CGAGGACGATGAAGATGCAGACACC | 120 | 205 |
| HPV45REF_1_24 | GAAGGAATCCCTTTCGGAACGTTTA | | |
| | AGTGCGTTACAGGACAAAATACTAG | | |
| | ACCACTATGAAAATGACA | | |
| | GTAAAGACATAAACAGCCAAATAAG | | |
| | TT | | |
| 129911_5270179_ | ATTGGCAACTTATACGTTTGGAAAA | 120 | 206 |
| HPV45REF_1_25 | TGCAATACTATTTACAGCAAGGGAA | | |
| | CATGGTATTACCAAACTAAACCACC | | |
| | AGGTGGTGCCTCCTATTAA | | |
| | CATTTCAAAAAGCAAAGCACATAAA | | |
| | G | | |
| 129911_5270179_ | CTATTGAACTGCAAATGGCCTTAAA | 120 | 207 |
| HPV45REF126 | GGGCCTTGCACAAAGCAAGTATAAC | | |
| | AATGAGGAATGGACACTGCAAGATA | | |
| | CATGCGAGGAACTATGGA | | |
| | ATACAGAACCGTCGCAGTGTTTTAA | | |
| | AA | | |
| 129911_5270179_ | AAGGCGGTAAAACCGTGCACGTATA | 120 | 208 |
| HPV45REF_1_27 | CTTTGATGGCAACAAGGACAACTGT | | |
| | ATGAACTATGTAGTATGGGACAGTA | | |
| | TATATTATATAACTGAGAC | | |
| | AGGGATATGGGACAAAACAGCAGCA | | |
| | T | | |
| 129911_5270179_ | GTGTTAGCTATTGGGGTGTATATTA | 120 | 209 |
| HPV45REF_1_28 | TATAAAAGATGGAGATACCACATAT | | |
| | TATGTACAATTTAAAAGCGAATGTG | | |
| | AGAAATATGGAAATAGTAAT | | |
| | ACGTGGGAAGTACAATATGGGGGCA | | |
| 129911_5270179_ | ATGTAATTGATTGTAATGACTCTAT | 120 | 210 |
| HPV45REF_1_29 | GTGCAGTACCAGTGACGACACGGTA | | |
| | TCCGCTACTCAGATTGTTAGACAGC | | |
| | TACAACACGCCTCCACGTC | | |
| | GACCCCCAAAACCGCATCCGTGGGC | | |
| | A | | |
| 129911_5270179_ | CCCCAAAACCCCACATCCAGACGCC | 120 | 211 |
| HPV45REF_1_30 | GGCTACTAAGCGACCTAGACAGTGT | | |
| | GGACTCACAGAGCAGCACCACGGAC | | |
| | GTGTCAACACCCACGT | | |
| | GCACAACCCGCTCCTGTGTTCAAGT | | |
| | ACAA | | |
| 129911_5270179_ | GTAACAACAAAAGAAGGAAAGTGTG | 120 | 212 |
| HPV45REF_1_31 | TAGTGGTAACACTACGCCTATAATA | | |
| | CACTTAAAAGGTGACAAAAACAGTT | | |
| | TGAAATGTTTAAGATATAG | | |
| | GCTACGCAAATATGCAGACCATTAC | | |
| | T | | |
| 129911_5270179_ | CAGAAATATCCTCCACCTGGCATTG | 120 | 213 |
| HPV45REF_1_32 | GACAGGTTGTAATAAAAACACTGGT | | |
| | ATATTAACTGTAACATATAATAGTG | | |
| | AGGTACAAAGAAATACCTTT | | |
| | TTGGATGTAGTTACTATTCCTAACA | | |
| 129911_5270179_ | GTGTACAAATCTCGGTGGGATACAT | 120 | 214 |
| HPV45REF_1_33 | GACTATATGAATCTGTATATTGTAT | | |
| | ACAGTATGTAACATTACTATGCTAT | | |
| | CTTTAGTGTTTTTATTGTGCT | | |
| | TTTCTGTGTGCCTTTATGTGTGCT | | |
| 129911_5270179_ | GCAATGTCCCGCTTGTGCAGTCTGT | 120 | 215 |
| HPV45REF_1_34 | CTATGTGTGTGCTTTTGCTTGGTTG | | |
| | TTGGTGTTTCTTTTTATAGTTGTTA | | |
| | TTACATCCCCATTAACAGCA | | |
| | TTTGCTGTATACATTTGTTGCTATT | | |
| 129911_5270179_ | TACTACCTATGTTTGTATTACATAT | 118 | 216 |
| HPV45REF_1_35 | GCATGCTTTACACACCATACAATAA | | |
| | TTACTATAATGTACAGTACAGTGTA | | |
| | ACATACCTGTGATGTGCATGT | | |
| | TGttgtatttttgtatttttgt | | |
| 129911_5270180_ | tttttgtatttttgtattttATATG | 120 | 217 |
| HPV45REF_2_1 | TTTAATAAACCATGGTATCCCACCG | | |
| | TGCAGCACGTCGCAAGCGGGCCTCT | | |
| | GCAACTGACTTATATAGAACATGTA | | |
| | AGC | | |
| | AATCCGGTACGTGCCCC | | |
| 129911_5270180_ | CCCTGATGTTATTAACAAAGTGGAA | 120 | 218 |
| HPV45REF_2_2 | GGCACAACCTTAGCTGATAAAATTT | | |
| | TACAGTGGTCTAGCCTTGGGATATT | | |
| | TTTGGGTGGCCTTGGCATT | | |
| | GGTACCGGCAGTGGTTCTGGAGGCC | | |
| | G | | |
| 129911_5270180_ | GTACGGGCTATGTACCCTTAGGGGG | 120 | 219 |
| HPV45REF_2_3 | CAGGTCTAATACTGTTGTGGATGTT | | |
| | GGCCCCACTAGGCCACCTGTGGTTA | | |
| | TTGAACCTGTAGGGCCT | | |
| | ACTGATCCATCTATTGTTACGTTGG | | |
| | TAG | | |
| 129911_5270180_ | GAGGATTCCAGTGTTGTTGCCTCTG | 120 | 220 |
| HPV45REF_2_4 | GTGCTCCGGTTCCCACATTTACCGG | | |
| | AACCTCTGGGTTTGAAATTACGTCT | | |
| | TCTGGTACTACCACACCA | | |
| | GCTGTGTTGGACATCACACCTACCG | | |
| | TG | | |
| 129911_5270180_ | GGACTCTGTTTCTATTTCGTCAACT | 120 | 221 |
| HPV45REF_2_5 | AGTTTTACAAATCCTGCATTTTCTG | | |
| | ATCCCTCTATTATTGAGGTGCCCCA | | |
| | AACAGGGGAGGTATCAGGTA | | |
| | ATATATTTGTTGGTACACCAACATC | | |
| 129911_5270180_ | CGGGCAGCCATGGATATGAGGAAAT | 120 | 222 |
| HPV45REF_2_6 | ACCTTTACAAACATTTGCATCTTCT | | |
| | GGGTCAGGTACGGAACCCATTAGTA | | |
| | GTACCCCCCTCCCTACTG | | |
| | TGCGGCGGGTACGGGGTCCCCGCCT | | |
| | GT | | |
| 129911_5270180_ | TATAGTAGGGCTAATCAACAGGTCC | 120 | 223 |
| HPV45REF_2_7 | GTGTGTCCACCTCACAGTTTTTAAC | | |
| | ACATCCCTCATCGTTGGTTACATTT | | |
| | GATAATCCAGCTTATGAGC | | |
| | CCCTGGACACCACACTATCCTTTGA | | |
| | G | | |
| 129911_5270180_ | GCCTACCAGTAATGTTCCTGATTCC | 120 | 224 |
| HPV45REF_2_8 | GATTTTATGGATATTATTCGTTTGC | | |
| | ATAGGCCAGCATTATCCTCTAGACG | | |
| | TGGCACTGTTAGATTTAGTA | | |
| | GATTGGGTCAAAGGGCAACCATGTT | | |
| 129911_5270180_ | TTACACGTAGTGGTAAACAAATAGG | 120 | 225 |
| HPV45REF_2_9 | GGGTAGGGTACATTTTTACCATGAT | | |
| | ATAAGCCCCATTGCTGCTACAGAGG | | |
| | AAATTGAATTGCAGCCTTT | | |
| | AATTAGTGCTACAAATGATAGTGAC | | |
| | C | | |
| 129911_5270180_ | CTGTTTGATGTATATGCAGACTTCC | 120 | 226 |
| HPV45REF_2_10 | CACCTCCTGCGTCCACTACACCTAG | | |
| | CACTATACACAAATCATTTACATAT | | |
| | CCAAAGTATTCCTTGACCAT | | |
| | GCCTTCTACTGCTGCATCCTCTTAC | | |
| 129911_5270180_ | CAGTAATGTTACAGTACCATTAACA | 120 | 227 |
| HPV45REF_2_11 | TCTGCATGGGATGTACCTATATATA | | |
| | CTGGCCCGGACATTATATTGCCATC | | |
| | CCATACTCCTATGTGGCCT | | |
| | AGTACATCTCCTACCAATGCTTCCA | | |
| | C | | |
| 129911_5270180_ | CCACCACCTATATAGGTATTCATGG | 120 | 228 |
| HPV45REF_2_12 | CACACAATATTATTTATGGCCATGG | | |
| | TATTATTATTTTCCTAAAAAACGTA | | |
| | AACGTATTCCCTATTTTTTTG | | |
| | CAGATGGCTTTGTGGCGGCCTAGT | | |
| 129911_5270180_ | TGACAGTACGGTATATCTTCCACCA | 120 | 229 |
| HPV45REF_2_13 | CCTTCTGTGGCCAGAGTTGTCAGCA | | |
| | CTGATGATTATGTGTCTCGCACAAG | | |
| | CATATTTTATCATGCAGGC | | |
| | AGTTCCCGATTATTAACTGTAGGCA | | |
| | A | | |
| 1299115270180_ | ATCCATATTTTAGGGTTGTACCTAA | 120 | 230 |
| HPV45REF_2_14 | TGGTGCAGGTAATAAACAGGCTGTT | | |
| | CCTAAGGTATCCGCATATCAGTATA | | |
| | GGGTGTTTAGAGTAGCTTT | | |
| | ACCCGATCCTAATAAATTTGGATTA | | |
| | C | | |
| 129911_5270180_ | CCTGATTCTACTATATATAATCCTG | 120 | 231 |
| HPV45REF_2_15 | AAACACAACGTTTGGTTTGGGCATG | | |
| | TGTAGGTATGGAAATTGGTCGTGGG | | |
| | CAGCCTTTAGGTATTGGCC | | |
| | TAAGTGGCCATCCATTTTATAATAA | | |
| | A | | |
| 129911_5270180_ | ATTGGATGATACAGAAAGTGCTCAT | 120 | 232 |
| HPV45REF_2_16 | GCAGCTACAGCTGTTATTACGCAGG | | |
| | ATGTTAGGGATAATGTGTCAGTTGA | | |
| | TTATAAGCAAACACAGCTG | | |
| | TGTATTTTAGGTTGTGTACCTGCTA | | |
| | T | | |
| 129911_5270180_ | TTGGTGAGCACTGGGCCAAGGGCAC | 120 | 233 |
| HPV45REF_2_17 | ACTTTGTAAACCTGCACAATTGCAA | | |
| | CCTGGTGACTGTCCTCCTTTGGAAC | | |
| | TTAAAAACACCATTATTG | | |
| | AGGATGGTGATATGGTGGATACAGG | | |
| | TT | | |
| 129911_5270180_ | TATGGGGCAATGGATTTTAGTACAT | 120 | 234 |
| HPV45REF_2_18 | TGCAGGATACAAAGTGCGAGGTTCC | | |
| | ATTAGACATTTGTCAATCCATCTGT | | |
| | AAATATCCAGATTATTTGCA | | |
| | AATGTCTGCTGATCCCTATGGGGAT | | |
| 129911_5270180_ | TTCTATGTTTTTTTCCTACGCUGTG | 120 | 235 |
| HPV45REF_2_19 | AACAACTGTTTGCAAGACATTTTTG | | |
| | GAATAGGGCAGGTGTTATGGGTGAC | | |
| | ACAGTACCTACGGACCTA | | |
| | TATATTAAAGGCACTAGCGCTAATA | | |
| | T | | |
| 129911_5270180_ | TGCGTGAAACCCCTGGCAGTTGTGT | 120 | 236 |
| HPV45REF_2_20 | GTATTCCCCTTCTCCCAGTGGCTCT | | |
| | ATTATTACTTCTGATTCTCAATTAT | | |
| | TTAATAAGCCATATTGGTTA | | |
| | CATAAGGCCCAGGGCCATAACAATG | | |
| 129911_5270180_ | GGTATTTGTTGGCATAATCAGTTGT | 120 | 237 |
| HPV45REF_2_21 | TTGTTACTGTAGTGGACACTACCCG | | |
| | CAGTACTAATTTAACATTATGTGCC | | |
| | TCTACACAAAATCCTGTGCC | | |
| | AAGTACATATGACCCTACTAAGTTT | | |
| 129911_5270180_ | TAAGCAGTATAGTAGACATGTGGAG | 120 | 238 |
| HPV45REF_2_22 | GAATATGATTTACAGTTTATTTTTC | | |
| | AGTTGTGCACTATTACTTTAACTGC | | |
| | AGAGGTTATGTCATATATCC | | |
| | ATAGTATGAATAGTAGTATATTAGA | | |
| 129911_5270180_ | AAAATTGGAATTTTGGTGTCCCTCC | 120 | 239 |
| HPV45REF_2_23 | ACCACCTACTACAAGTTTGGTGGAT | | |
| | ACATATCGTTTTGTGCAATCAGTTG | | |
| | CTGTTACCTGTCAAAAGGAT | | |
| | ACTACACCTCCAGAAAAGCAGGATC | | |
| 129911_5270180_ | CCATATGATAAATTAAAGTTTTGGA | 120 | 240 |
| HPV45REF_2_24 | CTGTTGACCTAAAGGAAAAATTTTC | | |
| | CTCCGATTTGGATCAATATCCCCTT | | |
| | GGTCGAAAGTTTTTAGTTCA | | |
| | GGCTGGGTTACGTCGTAGGCCTACC | | |
| 129911_5270180_ | CATAGGACCTCGTAAGCGTCCTGCT | 120 | 241 |
| HPV45REF_2_25 | GCTTCCACGTCTACTGCATCTACTG | | |
| | CATCTAGGCCTGCCAAACGTGTACG | | |
| | TATACGTAGTAAGAAATA | | |
| | ATATGTTAGCACATAtatgtatgtt | | |
| | tg | | |
| 1299115270181_ | gaatgtGCCTTGTGGCATGTATGGT | 120 | 242 |
| HPV45REF_3_1 | GTTACTGTACATAATTGTGGTATTA | | |
| | AATAAAGTATGCTAATAGTGTTGTG | | |
| | TAGGGTTGCACCCTTGTGAGT | | |
| | AACAATACTATTTGTGTGTATGTG | | |
| 129911_5270181_ | TATTGCTTTGTACCCTATATTCTTT | 120 | 243 |
| HPV45REF_3_2 | CCTGTATTTCAAGTTATAAACTTGC | | |
| | ATACTACACAGCATCCATTTTACTT | | |
| | ATAATCCTCCATTTTGCTGTGC | | |
| | AACCGATTTCGGTTGCCTGTGGC | | |
| 129911_5270181_ | TTATATGTGACCTTTTAAACATAAT | 120 | 244 |
| HPV45REF_3_3 | ACCTAAACTGGCACATTTACAACCC | | |
| | CTACATAGTTTAACCTACTGGCGCG | | |
| | CCTTCTTGGCGTACATGTGG | | |
| | CACACCTGGTATTAGTCATTTTCCT | | |
| 129911_5270181_ | GTCCAGGTGTACTAAAACAATGGCT | 120 | 245 |
| HPV45REF_3_4 | TGCACAACTGTATCCACACCCTATG | | |
| | TAATAAAACTGCTTTTAGGCACATA | | |
| | TTTTAGTCTGTTTTTACCTG | | |
| | TGCTAATTGTATAATTGGCGTGTAG | | |
| 129911_5270181_ | AACCACTTTCTTATCCAACAATCTG | 119 | 246 |
| HPV45REF_3_5 | TCTACTTGTTACATAAACTATAAAC | | |
| | TGACTCACTTATACATACATAGTTT | | |
| | ATGCAACCGAAAAAGGTTGGG | | |
| | CCCTATAACACATACCTTTTCTT | | |

| HPV52_SingleStrand_ | | | SEQ |
|---|---|---|---|
| SequenceName | Sequence | Length | ID#: |
| 133445_5511603_ | TAAATTATAATCTTATACTAGTAAA | 120 | 247 |
| HPV52REF.1(HPV52)_1_1 | AAATAGGGTGTAACCGAAAACGGTC | | |
| | AGACCGAAACCGGTGTATATATATA | | |
| | GAACACAGTG | | |
| | TAGCTAACGCACGGCCATGTTTGAG | | |
| | GATCCAGCAA | | |
| 133445_5511603_ | CACGACCCCGGACCCTGCACGAATT | 120 | 248 |
| HPV52REF.1(HPV52)_1_2 | GTGTGAGGTGCTGGAAGAATCGGTG | | |
| | CATGAAATAAGGCTGCAGTGTGTGC | | |
| | AGTGCAA | | |
| | AAAAGAGCTACAACGAAGAGAGGTA | | |
| | TACAAGTTTCTAT | | |
| 133445_5511603_ | TTACAGATTTACGAATAGTATATAG | 120 | 249 |
| HPV52REF.1(HPV52)_1_3 | AGACAATAATCCATATGGCGTGTGT | | |
| | ATTATGTGCCTACGCTTTTTATCTA | | |
| | AGATAAGTGAA | | |
| | TATAGGCATTATCAATATTCACTGT | | |
| | ATGGGAAAA | | |
| 133445_5511603_ | CATTAGAAGAGAGGGTAAAAAAACC | 120 | 250 |
| HPV52REF.1(HPV52)_1_4 | ATTAAGTGAAATAACTATTAGATGT | | |
| | ATAATTTGTCAAACGCCATTATGTC | | |
| | CTGAAGAAAA | | |
| | AGAAAGACATGTTAATGCAAACAAG | | |
| | CGATTTCATA | | |
| 133445_5511603_ | ATATTATGGGTCGTTGGACAGGGCG | 120 | 251 |
| HPV52REF.1(HPV52)_1_5 | CTGTTCAGAGTGTTGGAGACCCCGA | | |
| | CCTGTGACCCAAGTGTAACGTCATG | | |
| | CGTGGAG | | |
| | ACAAAGCAACTATAAAAGATTATAT | | |
| | ATTAGATCTGCAA | | |
| 133445_5511603_ | CCTGAAACAACTGACCTACACTGCT | 120 | 252 |
| HPV52REF.1(HPV52)_1_6 | ATGAGCAATTAGGTGACAGCTCAGA | | |
| | TGAGGAGGATACAGATGGTGTGGAC | | |
| | CGGCCAG | | |
| | ATGGACAAGCAGAACAAGCCACAAG | | |
| | CAATTACTACATT | | |
| 133445_5511603_ | GTGACATATTGTCACAGTTGTGATA | 120 | 253 |
| HPV52REF.1(HPV52)_1_7 | GCACACTACGGCTATGCATTCATAG | | |
| | CACTGCGACGGACCTTCGTACTCTA | | |
| | CAGCAAATG | | |
| | CTGTTGGGCACATTACAAGTTGTGT | | |
| | GCCCCGGCTGT | | |
| 133445_5511603_ | GCACGGCTATAAACAACCCTGCAAT | 120 | 254 |
| HPV52REF.1(HPV52)_1_8 | GGAGGACCCTGAAGGTACAGAGGGC | | |
| | GAAAGGGAGGGATGTACAGGCTGGT | | |
| | TTGAAGT | | |
| | AGAGGCAATAATAGAAAAACAAACA | | |
| | GGAGATAACATTT | | |
| 133445_5511603_ | CAGAGGACGAGGATGAAAATGCATA | 120 | 255 |
| HPV52REF.1(HPV52)_1_9 | TGATAGTGGAACAGATCTAATAGAT | | |
| | TTTATAGATGATTCAAATATAAATA | | |
| | ATGAACAGGC | | |
| | AGAACATGAGGCAGCCCGGGCATTG | | |
| | TTTAATGCAC | | |
| 133445_5511603_ | AGGAAGGGGAGGATGATTTACATGC | 120 | 256 |
| HPV52REF.1(HPV52)_1_10 | TGTGTCTGCAGTAAAACGAAAGTTT | | |
| | ACAAGCAGTCCGGAAAGTGCTGGGC | | |
| | AAGATGGT | | |
| | GTAGAAAAACATGGTAGTCCGCGTG | | |
| | CAAAACACATTT | | |
| 133445_5511603_ | GTGTAAATACAGAGTGTGTTTTACC | 120 | 257 |
| HPV52REF.1(HPV52)_1_11 | AAAACGCAAACCATGTCACGTAGAA | | |
| | GACAGCGGCTATGGCAATAGTGAAG | | |
| | TGGAAGCG | | |
| | CAGCAGATGGCAGACCAGGTAGACG | | |
| | GGCAAAATGGCG | | |
| 133445_5511603_ | ACTGGCAAAGTAACAGTAGTCAATC | 120 | 258 |
| HPV52REF.1(HPV52)_1_12 | AAGTGGGGTGGGGGCTAGTAATTCA | | |
| | GATGTAAGTTGTACTAGTATAGAGG | | |
| | ACAATGAG | | |
| | GAAAATAGTAATAGAACGCTAAAAA | | |
| | GCATACAAAATA | | |
| 133445_5511603_ | TTATGTGCGAAAATAGCATAAAAAC | 120 | 259 |
| HPV52REF.1(HPV52)_1_13 | AACTGTATTATTTAAATTTAAAGAA | | |
| | ACATATGGTGTTAGCTTTATGGAAT | | |
| | TAGTAAGACCA | | |
| | TTTAAAAGTAATAGAAGTAGTTGTA | | |
| | CAGATTGGT | | |
| 133445_5511603_ | GTATTATAGGAATGGGAGTAACACC | 120 | 260 |
| HPV52REF.1(HPV52)_1_14 | ATCAGTTGCAGAAGGATTAAAAGTA | | |
| | TTAATACAGCCCTATAGCATATATG | | |
| | CCCATTTGCA | | |
| | ATGTTTAACATGTGACAGAGGCGTG | | |
| | CTTATACTGC | | |
| 133445_5511603_ | TGCTAATTAGGTTTAAATGTGGAAA | 120 | 261 |
| HPV52REF.1(HPV52)_1_15 | AAACAGATTAACAGTGTCCAAACTA | | |
| | ATGTCACAGCTGTTAAATATACCAG | | |
| | AAACACATAT | | |
| | GGTAATAGAACCACCAAAATTACGA | | |
| | AGTGCTACCT | | |
| 133445_5511603_ | GTGCATTATATTGGTATAGAACAGG | 120 | 262 |
| HPV52REF.1(HPV52)_1_16 | TTTGTCTAATATTAGTGAGGTATAT | | |
| | GGTACCACCCCAGAATGGATAGAAC | | |
| | AACAAACAG | | |
| | TATTACAGCATAGCTTTGACAATAG | | |
| | CATATTCGATT | | |
| 133445_5511603_ | TTGGAGAAATGGTGCAATGGGCATA | 120 | 263 |
| HPV52REF.1(HPV52)_1_17 | TGATCATGATATAACAGATGATAGT | | |
| | GACATAGCATATAAATATGCACAGT | | |
| | TAGCAGATG | | |
| | TAAATAGCAATGCTGCAGCATTCCT | | |
| | AAAAAGCAATT | | |
| 133445_5511603_ | CGCAAGCAAAAATAGTAAAGGACTG | 120 | 264 |
| HPV52REF.1(HPV52)_1_18 | TGCAACCATGTGTAGACATTATAAA | | |
| | CGGGCAGAAAGAAAACATATGAATA | | |
| | TTGGACAAT | | |
| | GGATACAGTATAGATGTGATAGAAT | | |
| | AGATGATGGTG | | |
| 133445_5511603_ | GAGATTGGAGGCCTATAGTAAGATT | 120 | 265 |
| HPV52REF.1(HPV52)_1_19 | TTTAAGATATCAAGACATAGAATTT | | |
| | ACAGCCTTTTTAGACGCATTTAAAA | | |
| | AATTTTTAAAA | | |
| | GGTATACCTAAAAAAAATTGTTTAG | | |
| | TATTATATG | | |
| 133445_5511603_ | GACCTGCAAACACAGGAAAATCATA | 120 | 266 |
| HPV52REF.1(HPV52)_1_20 | TTTTGGAATGAGTTTAATTAGGTTC | | |
| | TTAAGTGGATGTGTAATATCCTATG | | |
| | TAAACTCAAA | | |
| | AAGCCATTTTTGGCTACAACCATTA | | |
| | ACAGATGCAA | | |
| 133445_5511603_ | AAGTGGGTATGATAGATGATGTAAC | 120 | 267 |
| HPV52REF.1(HPV52)_1_21 | ACCTATATGTTGGACATATATAGAT | | |
| | GATTATATGAGAAATGCACTGGATG | | |
| | GAAATGATAT | | |
| | ATCAGTAGATGTAAAGCATAGAGCC | | |
| | TTAGTACAAA | | |
| 133445_5511603_ | TAAAATGCCCACCATTAATTTTAAC | 120 | 268 |
| HPV52REF.1(HPV52)_1_22 | AACAAATACAAATGCAGGAACAGAT | | |
| | CCTAGGTGGCCATATTTACATAGTA | | |
| | GATTGGTTGT | | |
| | GTTTCATTTCAAAAACCCATTTCCA | | |
| | TTTGATGAAA | | |
| 133445_5511603_ | ATGGCAATCCTATATATGAAATTAA | 120 | 269 |
| HPV52REF.1(HPV52)_1_23 | CAACGAAAATTGGAAATCCTTTTTC | | |
| | TCAAGGACGTGGTGCAAATTAGATT | | |
| | TAATACAGGA | | |
| | AGAGGACAAGGAAAACGATGGAGTC | | |
| | GATACCGGCA | | |
| 133445_5511603_ | CGTTTAAATGCAGTGCAGGAAAAAA | 120 | 270 |
| HPV52REF.1(HPV52)_1_24 | TACTAGATCTATACGAAGCTGATAG | | |
| | TAATGACCTAAACGCACAAATTGAA | | |
| | CATTGGAAAT | | |
| | TGACTCGAATGGAATGTGTTTTGTT | | |
| | TTACAAAGCA | | |
| 133445_5511603_ | AAGGAACTGGGAATAACTCATATAG | 120 | 271 |
| HPV52REF.1(HPV52)_1_25 | GCCACCAGGTGGTGCCACCAATGGC | | |
| | AGTGTCTAAGGCAAAGGCCTGCCAA | | |
| | GCTATTG | | |
| | AACTACAATTGGCATTGGAGGCATT | | |
| | AAACAAAACACAA | | |
| 1334455511603_ | TATAGCACAGATGGATGGACATTAC | 120 | 272 |
| HPV52REF.1(HPV52)_1_26 | AACAAACAAGTCTAGAAATGTGGCG | | |
| | TGCAGAACCACAAAAATACTTTAAA | | |
| | AAACATGGG | | |
| | TATACAATAACAGTGCAATACGATA | | |
| | ATGATAAAAAC | | |
| 1334455511603_ | AATACTATGGATTATACAAACTGGA | 120 | 273 |
| HPV52REF.1(HPV52)_1_27 | AGGAAATTTATTTACTTGGTGAGTG | | |
| | TGAATGTACAATTGTAGAAGGACAA | | |
| | GTAGATTACT | | |
| | ATGGGTTATATTATTGGTGTGATGG | | |
| | AGAAAAAATA | | |
| 133445_5511603_ | TATTTTGTAAAATTTAGTAACGATG | 120 | 274 |
| HPV52REF.1(HPV52)_1_28 | CAAAGCAATATTGTGTAACAGGAGT | | |
| | ATGGGAAGTACATGTGGGTGGTCAG | | |
| | GTAATTGTTT | | |
| | GTCCTGCATCTGTATCTAGTAACGA | | |
| | AGTATCCACT | | |
| 133445_5511603_ | ACTGAAACTGCTGTCCACCTATGCA | 120 | 275 |
| HPV52REF.1(HPV52)_1_29 | CCGAAACCTCCAAGACCTCCGCAGT | | |
| | GTCCGTGGGTGCCAAAGACACACAC | | |
| | CTACAAC | | |
| | CACCACAGAAACGACGACGACCAGA | | |
| | CGTCACAGACTCC | | |
| 133445_5511603_ | AGAAACACCAAGTACCCCAACAACC | 120 | 276 |
| HPV52REF.1(HPV52)_1_30 | TTTTGCGGGGACAACAATCCGTGGA | | |
| | CAGTACTACACGGGGACTCGTCACT | | |
| | GCAACTGA | | |
| | GTGCACAAACAAAGGACGGGTTGCA | | |
| | CATACAACTTGT | | |
| 1334455511603_ | ACTGCACCTATAATACACCTAAAAG | 120 | 277 |
| HPV52REF.1(HPV52)_1_31 | GTGATCCTAATAGTTTAAAATGTTT | | |
| | AAGATATAGGGTAAAAACACATAAA | | |
| | AGTTTGTATGT | | |
| | TCAAATTTCATCTACCTGGCATTGG | | |
| | ACCAGTAAT | | |
| 1334455511603_ | GAATGTACAAATAATAAACTAGGTA | 120 | 278 |
| HPV52REF.1(HPV52)_1_32 | TTGTAACAATAACGTACAGTGATGA | | |
| | AACACAACGTCAACAATTTTTAAAA | | |
| | ACTGTTAAAAT | | |
| | ACCAAATACTGTGCAAGTTATACAA | | |
| | GGTGTCatg | | |
| 133445_5511604_ | tatatatgtatatgtgtatgGTAAA | 120 | 279 |
| HPV52REF.1(HPV52)_2_1 | CACCCAACACAAGCCAATATTGCTG | | |
| | CTATTGTGTATATATAACAATGTTA | | |
| | GGATTATTTGTATTTTGTT | | |
| | TTATTTTGCTTATGGTGTTTTGTGC | | |
| | A | | |
| 1334455511604_ | GTGCTTAGGCCGCTCTTGCTATCTA | 120 | 280 |
| HPV52REF.1(HPV52)_2_2 | TATCGGTGTATGCGCAGGTGTTGGT | | |
| | GCTGGTGCTTTTGCTATGGGTATCT | | |
| | ATTGGGTC | | |
| | ACCATTTAAAGTGTTTTTTTTGTAC | | |
| | CTACTGTTTTTA | | |
| 1334455511604_ | TATTTTCCAATGTTTTGTATTCACT | 120 | 281 |
| HPV52REF.1(HPV52)_2_3 | GTCATGCACAGTATTTGGCACAACT | | |
| | GCAATAACTGTACATGTAGATTGGC | | |
| | TACATGCATAT | | |
| | ATGCAAAATATACTTTTTCACTTTT | | |
| | GTAGTTTGT | | |
| 1334455511604_ | CTAATAAATACTTTTATATTTTTTA | 120 | 282 |
| HPV52REF.1(HPV52)_2_4 | ATAGCTTGTCGCAATGAGATACAGA | | |
| | CGGTCTACACGGCACAAACGTGCTT | | |
| | CTGCAACACA | | |
| | GCTATATCAAACATGCAAAGCCTCT | | |
| | GGCACCTGCC | | |
| 1334455511604_ | CCCCCGATGTTATTCCTAAAGTGGA | 120 | 283 |
| HPV52REF.1(HPV52)_2_5 | AGGCACAACTATTGCAGATCAACTT | | |
| | TTAAAATATGGCAGCCTAGGGGTGT | | |
| | TTTTTGGAG | | |
| | GTTTGGGTATAGGTACAGGTGCAGG | | |
| | CTCTGGTGGTA | | |
| 1334455511604_ | GGGCAGGCTATGTGCCATTGTCCAC | 120 | 284 |
| HPV52REF.1(HPV52)_2_6 | TCGTCCTCCCACTAGTAGTATTACC | | |
| | ACGTCCACCATTCGTCCCCCTGTAA | | |
| | CTGTAGAA | | |
| | CCCATTGGTCCCTTAGAACCATCTA | | |
| | TAGTTTCTATGA | | |
| 1334455511604_ | TAGAAGAAACAACATTTATTGAGTC | 120 | 285 |
| HPV52REF.1(HPV52)_2_7 | TGGCGCACCTGCTCCATCTATTCCA | | |
| | TCAGCAACAGGGTTTGATGTTACAA | | |
| | CATCTGCAA | | |
| | ATAATACTCCTGCAATAATTAATGT | | |
| | AACATCTATAG | | |
| 1334455511604_ | GTGAATCATCTGTACAATCAGTTTC | 120 | 286 |
| HPV52REF.1(HPV52)_2_8 | TACACATTTAAATCCTACATTCACT | | |
| | GAACCATCTATAATACAGCCCCCGG | | |
| | CACCTGCAGA | | |
| | AGCATCTGGTCATGTATTGTTTTCT | | |
| | AGTCCAACTA | | |
| 1334455511604_ | TTAGTACACACACCTATGAAGAAAT | 120 | 287 |
| HPV52REF.1(HPV52)_2_9 | CCCTATGGATACATTTGTTACCTCT | | |
| | ACTGACAGCAGCAGTGTAACAAGTA | | |
| | GTACACCTAT | | |
| | TCCAGGGTCTCGCCCTACGACACGC | | |
| | CTTGGTTTAT | | |
| 1334455511604_ | ATAGCCGTGCCACACAACAGGTTAA | 120 | 288 |
| HPV52REF.1(HPV52)_2_10 | GGTAGTCGACCCTGCTTTTATGTCA | | |
| | TCACCACAGAAATTAGTAACATATA | | |
| | ACAATCCTG | | |
| | TTTTTGAGGGCGTTGATACAGATGA | | |
| | AACTATAATTT | | |
| 1334455511604_ | TTGATCGTTCACAACTTTTACCTGC | 120 | 289 |
| HPV52REF.1(HPV52)_2_11 | ACCGGATCCTGATTTTTTAGACATT | | |
| | ATAGCTTTGCATAGGCCTGCATTAA | | |
| | CCTCTCGAAG | | |
| | AGGTACTGTTAGGTTTAGCAGGCTT | | |
| | GGTAATAAGG | | |
| 1334455511604_ | CCACCCTACGTACACGTAGTGGAAA | 120 | 290 |
| HPV52REF.1(HPV52)_2_12 | ACAAATTGGGGCACGGGTACATTAT | | |
| | TATCATGATATTAGTCCTATCCAGC | | |
| | CTGCTGAAG | | |
| | TTCAGGAAGACATAGAATTGCAACC | | |
| | TTTATTACCAC | | |
| 1334455511604_ | AGTCTGTGTCCCCTTACACTATTAA | 120 | 291 |
| HPV52REF.1(HPV52)_2_13 | TGATGGTTTGTATGATGTGTATGCA | | |
| | GATTCTTTGCAGCAACCCACGTTTC | | |
| | ACTTACCTTC | | |
| | CACACTTTCTACCCATAATAATACT | | |
| | TTCACTGTAC | | |
| 133445_5511604_ | CTATTAATAGTGGTATTGACTTTGT | 120 | 292 |
| HPV52REF.1(HPV52)_2_14 | ATATCAACCCACTATGTCCATTGAG | | |
| | TCAGGTCCTGACATTCCATTACCTT | | |
| | CGTTACCCAC | | |
| | ACATACTCCTTTTGTTCCTATAGCC | | |
| | CCTACAGCTC | | |
| 133445_5511604_ | CATCTACATCTATTATTGTTGATGG | 120 | 293 |
| HPV52REF.1(HPV52)_2_15 | TACAGATTTTATTTTACATCCTAGT | | |
| | TATTTTTTACTACGTCGCAGGCGTA | | |
| | AACGTTTTCCA | | |
| | TATTTTTTTACAGATGTCCGTGTGG | | |
| | CGGCCTAGT | | |
| 1334455511604_ | GAGGCCACTGTGTACCTGCCTCCTG | 120 | 294 |
| HPV52REF.1(HPV52)_2_16 | TACCTGTCTCTAAGGTTGTAAGCAC | | |
| | TGATGAGTATGTGTCTCGCACAAGC | | |
| | ATCTATTAT | | |
| 1334455511604_ | CCCTATTTTTCTATTAAAAACACCA | 120 | 295 |
| HPV52REF.1(HPV52)_2_17 | GTAGTGGTAATGGTAAAAAAGTTTT | | |
| | AGTTCCCAAGGTGTCTGGCCTGCAA | | |
| | TACAGGGTAT | | |
| | TTAGAATTAAATTGCCGGACCCTAA | | |
| | TAAATTTGGT | | |
| 1334455511604_ | TTTCCAGATACATCTTTTTATAACC | 120 | 296 |
| HPV52REF.1(HPV52)_2_18 | CAGAAACCCAAAGGTTGGTGTGGGC | | |
| | CTGTACAGGCTTGGAAATTGGTAGG | | |
| | GGACAGCC | | |
| | TTTAGGTGTGGGTATTAGTGGGCAT | | |
| | CCTTTATTAAAC | | |
| 1334455511604_ | AAGTTTGATGATACTGAAACCAGTA | 120 | 297 |
| HPV52REF.1(HPV52)_2_19 | ACAAATATGCTGGTAAACCTGGTAT | | |
| | AGATAATAGGGAATGTTTATCTATG | | |
| | GATTATAAGC | | |
| | AGACTCAGTTATGCATTTTAGGATG | | |
| | CAAACCTCCT | | |
| 1334455511604_ | ATAGGTGAACATTGGGGTAAGGGAA | 120 | 298 |
| HPV52REF.1(HPV52)_2_20 | CCCCTTGTAATAATAATTCAGGAAA | | |
| | TCCTGGGGATTGTCCTCCCCTACAG | | |
| | CTCATTAAC | | |
| | AGTGTAATACAGGATGGGGACATGG | | |
| | TAGATACAGGA | | |
| 1334455511604_ | TTTGGTTGCATGGATTTTAATACCT | 120 | 299 |
| HPV52REF.1(HPV52)_2_21 | TGCAAGCTAGTAAAAGTGATGTGCC | | |
| | CATTGATATATGTAGCAGTGTATGT | | |
| | AAGTATCCAG | | |
| | ATTATTTGCAAATGGCTAGCGAGCC | | |
| | ATATGGTGAC | | |
| 1334455511604_ | AGTTTGTTCTTTTTTcTTAGACGTG | 120 | 300 |
| HPV52REF.1(HPV52)_2_22 | AGCAAATGTTTGTTAGACACTTTTT | | |
| | TAATAGGGCCGGTACCTTAGGTGAC | | |
| | CCTGTGCCAG | | |
| | GTGATTTATATATACAAGGGTCTAA | | |
| | CTCTGGCAAT | | |
| 1334455511604_ | ACTGCCACTGTACAAAGCAGTGCTT | 120 | 301 |
| HPV52REF.1(HPV52)_2_23 | TTTTTCCTACTCCTAGTGGTTCTAT | | |
| | GGTAACCTCAGAATCCCAATTATTT | | |
| | AATAAACCGT | | |
| | ACTGGTTACAACGTGCGCAGGGCCA | | |
| | CAATAATGGC | | |
| 1334455511604_ | ATATGTTGGGGCAATCAGTTGTTTG | 120 | 302 |
| HPV52REF.1(HPV52)_2_24 | TCACAGTTGTGGATACCACTCGTAG | | |
| | CACTAACATGACTTTATGTGCTGAG | | |
| | GTTAAAAAG | | |
| | GAAAGCACATATAAAAATGAAAATT | | |
| | TTAAGGAATAC | | |
| 1334455511604_ | CTTCGTCATGGCGAGGAATTTGATT | 120 | 303 |
| HPV52REF.1(HPV52)_2_25 | TACAATTTATTTTTCAATTGTGCAA | | |
| | AATTACATTAACAGCTGATGTTATG | | |
| | ACATACATTCA | | |
| | TAAGATGGATGCCACTATTTTAGAG | | |
| | GACTGGCAA | | |
| 1334455511604_ | TTTGGCCTTACCCCACCACCGTCTG | 120 | 304 |
| HPV52REF.1(HPV52)_2_26 | CATCTTTGGAGGACACATACAGATT | | |
| | TGTCACTTCTACTGCTATAACTTGT | | |
| | CAAAAAAACA | | |
| | CACCACCTAAAGGAAAGGAAGATCC | | |
| | TTTAAAGGAC | | |
| 1334455511604_ | TATATGTTTTGGGAGGTGGATTTAA | 120 | 305 |
| HPV52REF.1(HPV52)_2_27 | AAGAAAAGTTTTCTGCAGATTTAGA | | |
| | TCAGTTTCCTTTAGGTAGGAAGTTT | | |
| | TTGTTACAGG | | |
| | CAGGGCTACAGGCTAGGCCCAAACT | | |
| | AAAACGCCCT | | |
| 1334455511604_ | GCATCATCGGCCCCACGTACCTCCA | 120 | 306 |
| HPV52REF.1(HPV52)_2_28 | CAAAGAAGAAAAAGGTTAAAAGGTA | | |
| | ACCATTGTCTGTTGGGTAATTGTCT | | |
| | GTGTCATGT | | |
| | ATGTGTTGTGTATGTCAAACACAGG | | |
| | TTAAAAGGTAA | | |
| 1334455511604_ | CCATTGTTTGTTATGTAATTGTTTT | 120 | 307 |
| HPV52REF.1(HPV52)_2_29 | GTGTGTGTACTGTGTTGTTTGCATG | | |
| | TTATGTATGTGTGTGCATGTTTGTT | | |
| | GTATTTGTCAG | | |
| | TTCCTGTATGTATGTTTTGTGTATG | | |
| | TATTAATAA | | |
| 1334455511604_ | AGTACTGTATTTACTAAACTATTTA | 117 | 308 |
| HPV52REF.1(HPV52)_2_30 | TAGTAGTCTTATGTTATGTTATGGT | | |
| | TGCACCCACATGAGTAACAATACAG | | |
| | TTGCTCCTAAT | | |
| | CTATTGCATCTcctgccctaccctg | | |
| | tgtccc | | |
| 133445_5511605_ | ccctaccctgTGTCCTACTTTGTTA | 120 | 309 |
| HPV52REF.1(HPV52)_3_1 | CACTACTAATTAGCCTTATACTCTC | | |
| | CATTTTGTACCATTTTGTACTATCC | | |
| | ACCATTTTAAATCCT | | |
| | AACCGAATTCGGTTGGTCTTGGCAC | | |
| | AACTT | | |
| 133445_5511605_ | TGGTTGTCCTTGGCACAGTAACAAC | 120 | 310 |
| HPV52REF.1(HPV52)_3_2 | TATTTTTATATAAGTTTCAGCAAAC | | |
| | TGCTTAATCCTTTGGTTTCCTGCAG | | |
| | TCCACTGGTC | | |
| | TACACTTGTTGTCCCGCCTAAACTG | | |
| | ACTTCTTGCT | | |
| 133445_5511605_ | GACTCACAGGTCCTGCAGTGCAGCT | 120 | 311 |
| HPV52REF.1(HPV52)_3_3 | AAACAATACATTGCCTAACATTGCA | | |
| | TGTTTTAAACTGCTTTTAGGCACAT | | |
| | ATTTTATTTA | | |
| | AACTTTCAATGCACTAATTACAGTG | | |
| | TTGGCTTACA | | |
| 133445_5511605_ | CAAGTACATCCTACGCCAAATATGT | 119 | 312 |
| HPV52REF.1(HPV52)_3_4 | CTTGTAAAACATGATTAAATACTGT | | |
| | TACTCACCAGGTGTGCACTACACGA | | |
| | CCGGTTACG | | |
| | GTTACCGTACCCACAACCACTTTTT | | |
| | TTTATAATTA | | |

| HPV16_DoubleStrand_ | | | |
|---|---|---|---|
| SequenceName | sequence | Length | SEQ ID#: |
| 88160_3764094_ | ACTACAATAATTCATGTATAAAACT | 120 | 313 |
| HPV16_1_1 | AAGGGCGTAACCGAAATCGGTTGAA | | |
| | CCGAAACCGGTTAGTATAAAAGC | | |
| | AGACATTTTATGCACCAAAAGAGAA | | |
| | CTGCAATGTTTCAGGACCCACA | | |
| 88160_3764094_ | GGAGCGACCCAGAAAGTTACCACAG | 120 | 314 |
| HPV16_1_2 | TTATGCACAGAGCTGCAAACAACTA | | |
| | TACATGATATAATATTAGAATGT | | |
| | GTGTACTGCAAGCAACAGTTACTGC | | |
| | GACGTGAGGTATATGACTTTGC | | |
| 88160_3764094_ | TTTTCGGGATTTATGCATAGTATAT | 120 | 315 |
| HPV16_1_3 | AGAGATGGGAATCCATATGCTGTAT | | |
| | GTGATAAATGTTTAAAGTTTTATT | | |
| | CTAAAATTAGTGAGTATAGACATTA | | |
| | TTGTTATAGTTTGTATGGAAC | | |
| 88160_3764094_ | AACATTAGAACAGCAATACAACAAA | 120 | 316 |
| HPV16_1_4 | CCGTTGTGTGATTTGTTAATTAGGT | | |
| | GTATTAACTGTCAAAAGCCACTG | | |
| | TGTCCTGAAGAAAAGCAAAGACATC | | |
| | TGGACAAAAAGCAAAGATTCCA | | |
| 88160_3764094_ | TAATATAAGGGGTCGGTGGACCGGT | 120 | 317 |
| HPV16_1_5 | CGATGTATGTCTTGTTGCAGATCAT | | |
| | CAAGAACACGTAGAGAAACCCA | | |
| | GCTGTAATCATGCATGGAGATACAC | | |
| | CTACATTGCATGAATATATGTTA | | |
| 88160_3764094_ | GATTTGCAACCAGAGACAACTGATC | 120 | 318 |
| HPV16_1_6 | TCTACTGTTATGAGCAATTAAATGA | | |
| | CAGCTCAGAGGAGGAGGATGAA | | |
| | ATAGATGGTCCAGCTGGACAAGCAG | | |
| | AACCGGACAGAGCCCATTACAAT | | |
| 88160_3764094_ | ATTGTAACCTTTTGTTGCAAGTGTG | 120 | 319 |
| HPV16_1J | ACTCTACGCTTCGGTTGTGCGTACA | | |
| | AAGCACACACGTAGACATTCGTA | | |
| | CTTTGGAAGACCTGTTAATGGGCAC | | |
| | ACTAGGAATTGTGTGCCCCATC | | |
| 88160_3764094_ | TGTTCTCAGAAACCATAATCTACCA | 120 | 320 |
| HPV16_1_8 | TGGCTGATCCTGCAGGTACCAATGG | | |
| | GGAAGAGGGTACGGGATGTAAT | | |
| | GGATGGTTTTATGTAGAGGCTGTAG | | |
| | TGGAAAAAAAAACAGGGGATGCT | | |
| 88160_3764094_ | ATATCAGATGACGAGAACGAAAATG | 120 | 321 |
| HPV16_1_9 | ACAGTGATACAGGTGAAGATTTGGT | | |
| | AGATTTTATAGTAAATGATAATG | | |
| | ATTATTTAACACAGGCAGAAACAGA | | |
| | GACAGCACATGCGTTGTTTACT | | |
| 88160_3764094_ | GCACAGGAAGCAAAACAACATAGAG | 120 | 322 |
| HPV16_1_10 | ATGCAGTACAGGTTCTAAAACGAAA | | |
| | GTATTTGGGTAGTCCACTTAGT | | |
| | GATATTAGTGGATGTGTAGACAATA | | |
| | ATATTAGTCCTAGATTAAAAGCT | | |
| 88160_3764094_ | ATATGTATAGAAAAACAAAGTAGAG | 120 | 323 |
| HPV16_1_11 | CTGCAAAAAGGAGATTATTTGAAAG | | |
| | CGAAGACAGCGGGTATGGCAAT | | |
| | ACTGAAGTGGAAACTCAGCAGATGT | | |
| | TACAGGTAGAAGGGCGCCATGAG | | |
| 88160_3764094_ | ACTGAAACACCATGTAGTCAGTATA | 120 | 324 |
| HPV16_1_12 | GTGGTGGAAGTGGGGGTGGTTGCAG | | |
| | TCAGTACAGTAGTGGAAGTGG | | |
| | GGGAGAGGGTGTTAGTGAAAGACAC | | |
| | ACTATATGCCAAACACCACTTACA | | |
| 88160_3764094_ | AATATTTTAAATGTACTAAAAACTA | 120 | 325 |
| HPV16_1_13 | GTAATGCAAAGGCAGCAATGTTAGC | | |
| | AAAATTTAAAGAGTTATACGGGGT | | |
| | GAGTTTTTCAGAATTAGTAAGACCA | | |
| | TTTAAAAGTAATAAATCAACG | | |
| 88160_3764094_ | TGTTGCGATTGGTGTATTGCTGCAT | 120 | 326 |
| HPV16_1_14 | TTGGACTTACACCCAGTATAGCTGA | | |
| | CAGTATAAAAACACTATTACAAC | | |
| | AATATTGTTTATATTTACACATTCA | | |
| | AAGTTTAGCATGTTCATGGGGA | | |
| 88160_3764094_ | ATGGTTGTGTTACTATTAGTAAGAT | 120 | 327 |
| HPV16_1_15 | ATAAATGTGGAAAAAATAGAGAAAC | | |
| | AATTGAAAAATTGCTGTCTAAACT | | |
| | ATTATGTGTGTCTCCAATGTGTATG | | |
| | ATGATAGAGCCTCCAAAATTG | | |
| 88160_3764094_ | CGTAGTACAGCAGCAGCATTATATT | 120 | 328 |
| HPV16_1_16 | GGTATAAAACAGGTATATCAAATAT | | |
| | TAGTGAAGTGTATGGAGACACGC | | |
| | CAGAATGGATACAAAGACAAACAGT | | |
| | ATTACAACATAGTTTTAATGAT | | |
| 88160_3764094_ | TGTACATTTGAATTATCACAGATGG | 120 | 329 |
| HPV16_1_17 | TACAATGGGCCTACGATAATGACAT | | |
| | AGTAGACGATAGTGAAATTGCAT | | |
| | ATAAATATGCACAATTGGCAGACAC | | |
| | TAATAGTAATGCAAGTGCCTTT | | |
| 88160_3764094_ | CTAAAAAGTAATTCACAGGCAAAAA | 120 | 330 |
| HPV16_1_18 | TTGTAAAGGATTGTGCAACAATGTG | | |
| | TAGACATTATAAACGAGCAGAAA | | |
| | AAAAACAAATGAGTATGAGTCAATG | | |
| | GATAAAATATAGATGTGATAGG | | |
| 88160_3764094_ | GTAGATGATGGAGGTGATTGGAAGC | 120 | 331 |
| HPV16_1_19 | AAATTGTTATGTTTTTAAGGTATCA | | |
| | AGGTGTAGAGTTTATGTCATTTT | | |
| | TAACTGCATTAAAAAGATTTTTGCA | | |
| | AGGCATACCTAAAAAAAATTGC | | |
| 88160_3764094_ | ATATTACTATATGGTGCAGCTAACA | 120 | 332 |
| HPV16_1_20 | CAGGTAAATCATTATTTGGTATGAG | | |
| | TTTAATGAAATTTCTGCAAGGGTC | | |
| | TGTAATATGTTTTGTAAATTCTAAA | | |
| | AGCCATTTTTGGTTACAACCA | | |
| 88160_3764094_ | TTAGCAGATGCCAAAATAGGTATGT | 120 | 333 |
| HPV16_1_21 | TAGATGATGCTACAGTGCCCTGTTG | | |
| | GAACTACATAGATGACAATTTAA | | |
| | GAAATGCATTGGATGGAAATTTAGT | | |
| | TTCTATGGATGTAAAGCATAGA | | |
| 88160_3764094_ | CCATTGGTACAACTAAAATGCCCTC | 120 | 334 |
| HPV16_1_22 | CATTATTAATTACATCTAACATTAA | | |
| | TGCTGGTACAGATTCTAGGTGGC | | |
| | CTTATTTACATAATAGATTGGTGGT | | |
| | GTTTACATTTCCTAATGAGTTT | | |
| 88160_3764094_ | CCATTTGACGAAAACGGAAATCCAG | 120 | 335 |
| HPV16_1_23 | TGTATGAGCTTAATGATAAGAACTG | | |
| | GAAATCCTTTTTCTCAAGGACGT | | |
| | GGTCCAGATTAAGTTTGCACGAGGA | | |
| | CGAGGACAAGGAAAACGATGGA | | |
| 88160_3764094_ | GACTCTTTGCCAACGTTTAAATGTG | 120 | 336 |
| HPV16_1_24 | TGTCAGGACAAAATACTAACACATT | | |
| | ATGAAAATGATAGTACAGACCTA | | |
| | CGTGACCATATAGACTATTGGAAAC | | |
| | ACATGCGCCTAGAATGTGCTAT | | |
| 88160_3764094_ | TTATTACAAGGCCAGAGAAATGGGA | 120 | 337 |
| HPV16_1_25 | TTTAAACATATTAACCACCAGGTGG | | |
| | TGCCAACACTGGCTGTATCAAA | | |
| | GAATAAAGCATTACAAGCAATTGAA | | |
| | CTGCAACTAACGTTAGAAACAAT | | |
| 88160_3764094_ | ATATAACTCACAATATAGTAATGAA | 120 | 338 |
| HPV16_1_26 | AAGTGGACATTACAAGACGTTAGCC | | |
| | TTGAAGTGTATTTAACTGCACCA | | |
| | ACAGGATGTATAAAAAAACATGGAT | | |
| | ATACAGTGGAAGTGCAGTTTGA | | |
| 88160_3764094_ | TGGAGACATATGCAATACAATGCAT | 120 | 339 |
| HPV16_1_27 | TATACAAACTGGACACATATATATA | | |
| | TTTGTGAAGAAGCATCAGTAACT | | |
| | GTGGTAGAGGGTCAAGTTGACTATT | | |
| | ATGGTTTATATTATGTTCATGA | | |
| 88160_3764094_ | AGGAATACGAACATATTTTGTGCAG | 120 | 340 |
| HPV16_1_28 | TTTAAAGATGATGCAGAAAAATATA | | |
| | GTAAAAATAAAGTATGGGAAGTT | | |
| | CATGCGGGTGGTCAGGTAATATTAT | | |
| | GTCCTACATCTGTGTTTAGCAG | | |
| 88160_3764094_ | CAACGAAGTATCCTCTCCTGAAATT | 120 | 341 |
| HPV16_1_29 | ATTAGGCAGCACTTGGCCAACCACC | | |
| | CCGCCGCGACCCATACCAAAGC | | |
| | CGTCGCCTTGGGCACCGAAGAAACA | | |
| | CAGACGACTATCCAGCGACCAAG | | |
| 88160_3764094_ | ATCAGAGCCAGACACCGGAAACCCC | 120 | 342 |
| HPV16_1_30 | TGCCACACCACTAAGTTGTTGCACA | | |
| | GAGACTCAGTGGACAGTGCTC | | |
| | CAATCCTCACTGCATTTAACAGCTC | | |
| | ACACAAAGGACGGATTAACTGTAA | | |
| 88160_3764094_ | TAGTAACACTACACCCATAGTACAT | 120 | 343 |
| HPV16_1_31 | TTAAAAGGTGATGCTAATACTTTAA | | |
| | AATGTTTAAGATATAGATTTAAAA | | |
| | AGCATTGTACATTGTATACTGCAGT | | |
| | GTCGTCTACATGGCATTGGAC | | |
| 88160_3764094_ | AGGACATAATGTAAAACATAAAAGT | 120 | 344 |
| HPV16_1_32 | GCAATTGTTACACTTACATATGATA | | |
| | GTGAATGGCAACGTGACCAATTTT | | |
| | TGTCTCAAGTTAAAATACCAAAAAC | | |
| | TATTACAGTGTCTACTGGATT | | |
| 88160_3764094_ | TATGTCTATATGACAAATCTTGATA | 120 | 345 |
| HPV16_1_33 | CTGCATCCACAACATTACTGGCGTG | | |
| | CTTTTTGCTTTGCTTTTGTGTGCT | | |
| | TTTGTGTGTCTGCCTATTAATACGT | | |
| | CCGCTGCTTTTGTCTGTGTCT | | |
| 88160_3764094_ | ACATACACATCATTAATAATATTGG | 120 | 346 |
| HPV16_1_34 | TATTACTATTGTGGATAACAGCAGC | | |
| | CTCTGCGTTTAGGTGTTTTATTGT | | |
| | ATATATTATATTTGTTTATATACCA | | |
| | TTATTTTTAATACATACACAT | | |
| 88160_3764095_ | tttttttttttgtttgtttgtttgt | 120 | 347 |
| HPV16_2_1 | tttttAATAAACTGTTATTACTTAA | | |
| | CAATGCGACACAAACGTTCTGCAAA | | |
| | ACGCACAAAACGTG | | |
| | CATCGGCTACCCAACTTTATAAAAC | | |
| | ATGCAA | | |
| 88160_3764095_ | ACAGGCAGGTACATGTCCACCTGAC | 120 | 348 |
| HPV16_2_2 | ATTATACCTAAGGTTGAAGGCAAAA | | |
| | CTATTGCTGATCAAATATTACAA | | |
| | TATGGAAGTATGGGTGTATTTTTTG | | |
| | GTGGGTTAGGAATTGGAACAGG | | |
| 88160_3764095_ | GTCGGGTACAGGCGGACGCACTGGG | 120 | 349 |
| HPV16_2_3 | TATATTCCATTGGGAACAAGGCCTC | | |
| | CCACAGCTACAGATACACTTG | | |
| | CTCCTGTAAGACCCCCTTTAACAGT | | |
| | AGATCCTGTGGGCCCTTCTGATCC | | |
| 88160_3764095_ | TTCTATAGTTTCTTTAGTGGAAGAA | 120 | 350 |
| HPV16_2_4 | ACTAGTTTTATTGATGCTGGTGCAC | | |
| | CAACATCTGTACCTTCCATTCCCC | | |
| | CAGATGTATCAGGATTtagtattac | | |
| | tacttcaactgataccacacc | | |
| 88160_3764096_ | tattagatattaataatactgttac | 120 | 351 |
| HPV16_3_1 | tactgttactacACATAATAATCCC | | |
| | ACTTTCACTGACCCATCTGTATTGC | | |
| | AGCCTCCAACAC | | |
| | CTGCAGAAACTGGAGGGCATTTTAC | | |
| | ACTTTCAT | | |
| 88160_3764096_ | CATCCACTATTAGTACACATAATTA | 120 | 352 |
| HPV16_3_2 | TGAAGAAATTCCTATGGATACATTT | | |
| | ATTGTTAGCACAAACCCTAACACA | | |
| | GTAACTAGTAGCACACCCATACCAG | | |
| | GGTCTCGCCCAGTGGCACGCC | | |
| 88160_3764096_ | TAGGATTATATAGTCGCACAACACA | 120 | 353 |
| HPV16_3_3 | ACAGGTTAAAGTTGTAGACCCTGCT | | |
| | TTTGTAACCACTCCCACTAAACT | | |
| | TATTACATATGATAATCCTGCATAT | | |
| | GAAGGTATAGATGTGGATAATA | | |
| 88160_3764096_ | CATTATATTTTTCTAGTAATGATAA | 120 | 354 |
| HPV16_3_4 | TAGTATTAATATAGCTCCAGATCCT | | |
| | GACTTTTTGGATATAGTTGCTTTAC | | |
| | ATAGGCCAGCATTAACCTCTAGGCG | | |
| | TACTGGCATTAGGTACAGTA | | |
| 88160_3764096_ | GAATTGGTAATAAACAAACACTACG | 120 | 355 |
| HPV16_3_5 | TACTCGTAGTGGAAAATCTATAGGT | | |
| | GCTAAGGTACATTATTATTATGAT | | |
| | TTAAGTACTATTGATCCTGCAGAAG | | |
| | AAATAGAATTACAAACTATAA | | |
| 88160_3764096_ | CACCTTCTACATATACTACCACTTC | 120 | 356 |
| HPV16_3_6 | ACATGCAGCCTCACCTACTTCTATT | | |
| | AATAATGGATTATATGATATTTAT | | |
| | GCAGATGACTTTATTACAGATACTT | | |
| | CTACAACCCCGGTACCATCTG | | |
| 88160_3764096_ | TACCCTCTACATCTTTATCAGGTTA | 120 | 357 |
| HPV16_3_7 | TATTCCTGCAAATACAACAATTCCT | | |
| | TTTGGTGGTGCATACAATATTCCT | | |
| | TTAGTATCAGGTCCTGATATACCCA | | |
| | TTAATATAACTGACCAAGCTC | | |
| 88160_3764096_ | CTTCATTAATTCCTATAGTTCCAGG | 120 | 358 |
| HPV16_3_8 | GTCTCCACAATATACAATTATTGCT | | |
| | GATGCAGGTGACTTTTATTTACAT | | |
| | CCTAGTTATTACATGTTACGAAAAC | | |
| | GACGTAAACGTTTACCATATT | | |
| 88160_3764096_ | TTTTTTCAGATGTCTCTTTGGCTGC | 120 | 359 |
| HPV16_3_9 | CTAGTGAGGCCACTGTCTACTTGCC | | |
| | TCCTGTCCCAGTATCTAAGGTTG | | |
| | TAAGCACGGATGAATATGTTGCACG | | |
| | CACAAACATATATTATCATGCA | | |
| 88160_3764096_ | GGAACATCCAGACTACTTGCAGTTG | 120 | 360 |
| HPV16_3_10 | GACATCCCTATTTTCCTATTAAAAA | | |
| | ACCTAACAATAACAAAATATTAGT | | |
| | TCCTAAAGTATCAGGATTACAATAC | | |
| | AGGGTATTTAGAATACATTTA | | |
| 88160_3764096_ | CCTGACCCCAATAAGTTTGGTTTTC | 120 | 361 |
| HPV16_3_11 | CTGACACCTCATTTTATAATCCAGA | | |
| | TACACAGCGGCTGGTTTGGGCCT | | |
| | GTGTAGGTGTTGAGGTAGGTCGTGG | | |
| | TCAGCCATTAGGTGTGGGCATT | | |
| 88160_3764096_ | AGTGGCCATCCTTTATTAAATAAAT | 120 | 362 |
| HPV16_3_12 | TGGATGACACAGAAAATGCTAGTGC | | |
| | TTATGCAGCAAATGCAGGTGTGG | | |
| | ATAATAGAGAATGTATATCTATGGA | | |
| | TTACAAACAAACACAATTGTGT | | |
| 88160_3764096_ | TTAATTGGTTGCAAACCACCTATAG | 120 | 363 |
| HPV16_3_13 | GGGAACACTGGGGCAAAGGATCCCC | | |
| | ATGTACCAATGTTGCAGTAAAT | | |
| | CCAGGTGATTGTCCACCATTAGAGT | | |
| | TAATAAACACAGTTATTCAGGAT | | |
| 88160_3764096_ | GGTGATATGGTTGATACTGGCTTTG | 120 | 364 |
| HPV16_3_14 | GTGCTATGGACTTTACTACATTACA | | |
| | GGCTAACAAAAGTGAAGTTCCAC | | |
| | TGGATATTTGTACATCTATTTGCAA | | |
| | ATATCCAGATTATATTAAAATG | | |
| 88160_3764096_ | GTGTCAGAACCATATGGCGACAGCT | 120 | 365 |
| HPV16_3_15 | TATTTTTTTATTTACGAAGGGAACA | | |
| | AATGTTTGTTAGACATTTATTTAA | | |
| | TAGGGCTGGTACTGTTGGTGAAAAT | | |
| | GTACCAGACGATTTATACATT | | |
| 88160_3764096_ | AAAGGCTCTGGGTCTACTGCAAATT | 120 | 366 |
| HPV16_3_16 | TAGCCAGTTCAAATTATTTTCCTAC | | |
| | ACCTAGTGGTTCTATGGTTACCT | | |
| | CTGATGCCCAAATATTCAATAAACC | | |
| | TTATTGGTTACAACGAGCACAG | | |
| 88160_3764096_ | GGCCACAATAATGGCATTTGTTGGG | 120 | 367 |
| HPV16_3_17 | GTAACCAACTATTTGTTACTGTTGT | | |
| | TGATACTACACGCAGTACAAATA | | |
| | TGTCATTATGTGCTGCCATATCTAC | | |
| | TTCAGAAACTACATATAAAAAT | | |
| 88160_3764096_ | ACTAACTTTAAGGAGTACCTACGAC | 120 | 368 |
| HPV16_3_18 | ATGGGGAGGAATATGATTTACAGTT | | |
| | TATTTTTCAACTGTGCAAAATAA | | |
| | CCTTAACTGCAGACGTTATGACATA | | |
| | CATACATTCTATGAATTCCACT | | |
| 88160_3764096_ | ATTTTGGAGGACTGGAATTTTGGTC | 120 | 369 |
| HPV16_3_19 | TACAACCTCCCCCAGGAGGCACACT | | |
| | AGAAGATACTTATAGGTTTGTAA | | |
| | CATCCCAGGCAATTGCTTGTCAAAA | | |
| | ACATACACCTCCAGCACCTAAA | | |
| 88160_3764096_ | GAAGATCCCCTTAAAAAATACACTT | 120 | 370 |
| HPV16_3_20 | TTTGGGAAGTAAATTTAAAGGAAAA | | |
| | GTTTTCTGCAGACCTAGATCAGTT | | |
| | TCCTTTAGGACGCAAATTTTTACTA | | |
| | CAAGCAGGATTGAAGGCCAAA | | |
| 88160_3764096_ | CCAAAATTTACATTAGGAAAACGAA | 120 | 371 |
| HPV16_3_21 | AAGCTACACCCACCACCTCATCTAC | | |
| | CTCTACAACTGCTAAACGCAAAA | | |
| | AACGTAAGCtgtaagtattgtatgt | | |
| | atgttgaattagtgttgtttgt | | |
| 88160_3764097_ | GTCATGCAACATAAATAAACTTATT | 120 | 372 |
| HPV16_4_2 | GTTTCAACACCTACTAATTGTGTTG | | |
| | TGGTTATTCATTGTATATAAACTAT | | |
| | ATTTGCTACATCCTGTTTTTGTTTT | | |
| | ATATATACTATATTTTGTAG | | |
| 88160_3764097_ | CGCCAGCGGCCATTTTGTAGCTTCA | 120 | 373 |
| HPV16_4_3 | ACCGAATTCGGTTGCATGCTTTTTG | | |
| | GCACAAAATGTGTTTTTTTAAAT | | |
| | AGTTCTATGTCAGCAACTATGGTTT | | |
| | AAACTTGTACGTTTCCTGCTTG | | |
| 88160_3764097_ | CCATGCGTGCCAAATCCCTGTTTTC | 120 | 374 |
| HPV16_4_4 | CTGACCTGCACTGCTTGCCAACCAT | | |
| | TCCATTGTTTTTTACACTGCACT | | |
| | ATGTGCAACTACTGAATCACTATGT | | |
| | ACATTGTGTCATATAAAATAAA | | |
| 88160_3764097_ | TCACTATGCGCCAACGCCTTACATA | 120 | 375 |
| HPV16_4_5 | CCGCTGTTAGGCACATATTTTTGGC | | |
| | TTGTTTTAACTAACCTAATTGCA | | |
| | TATTTGGCATAAGGTTTAAACTTCT | | |
| | AAGGCCAACTAAATGTCACCCT | | |
| 88160_3764097_ | AGTTCATACATGAACTGTGTAAAGG | 120 | 376 |
| HPV16_4_6 | TTAGTCATACATTGTTCATTTGTAA | | |
| | AACTGCACATGGGTGTGTGCAAA | | |
| | CCGTTTTGGGTTACACATTTACAAG | | |
| | CAACTTATATAATAATACTAA | | |
| 133919_5536530_ | TATCATGTATAGTTGTTTGCAGCTC | 120 | 377 |
| KP212157.1-HPV16- | TGTGCATAACTGTGGTAACTTTCTG | | |
| CNA138_1_2-REV | GGTCGCTCCTGTGGGTCCTGAAA | | |
| | CATTGCAGTTCTCTTTTGGTGCATA | | |
| | AAATGTCTGCTTTTATACTAAC | | |
| 133919_5536530_ | TTATCACATACAGCATATGGATTCC | 120 | 378 |
| KP212157.1-HPV16- | CATCTCTATATACTATGCATAAATC | | |
| CNA138_1_4-REV | CCGAAAAGCAAAGTCATATACCTC | | |
| | ACGTCGCAGTAACTGTTGCTTGCAG | | |
| | TACACACATTCTAATATTATA | | |
| 133919_5536530_ | AATACACCTAATTAACAAATCACAC | 120 | 379 |
| KP212157.1-HPV16- | AACGGTTTGTTGTATTGCTGTTCTA | | |
| CNA138_1_6-REV | ATGTTGTTCCATACAAACTATAAC | | |
| | AATAATGTCTATACTCACTAATTTT | | |
| | AGAATAAAACTTTAAACATTT | | |
| 133919_5536530_ | TTGATGATCTGCAACAAGACATACA | 120 | 380 |
| KP212157.1-HPV16- | TCGACCGGTCCACCGACCCCTTATA | | |
| CNA138_1_8-REV | TTATGGAATCTTTGCTTTTTGTC | | |
| | CAGATGTCTTTGCTTTTCTTCAGGA | | |
| | CACAGTGGCTTTTGACAGTTAA | | |
| 133919_5536530_ | GTCATTTAATTGCTCATAACAGTAG | 120 | 381 |
| KP212157.1-HPV16- | AGATCAGTTGTCTCTGGTTGCAAAT | | |
| CNA138_1_10-REV | CTAACATATATTCATGCAATGTAG | | |
| | GTGTATCTCCATGCATGATTACAGC | | |
| | TGGGTTTCTCTACGTGTTCTT | | |
| 133919_5536530_ | GTACGCACAACCGAAGCGTAGAGTC | 120 | 382 |
| KP212157.1-HPV16- | ACACTTGCAACAAAAGGTTACAATA | | |
| CNA138_1_12-REV | TTGTAATGGGCTCTGTCCGGTT | | |
| | CTGCTTGTCCAGCTGGACCATCTAT | | |
| | TTCATCCTCCTCCTCTGAGCTGT | | |
| 133919_5536530_ | TTGGTACCTGCAGGATCAGCCATGG | 120 | 383 |
| KP212157.1-HPV16- | TAGATTATGGTTTCTGAGAACAGAT | | |
| CNA138_1_14-REV | GGGGCACACAATTCCTAGTGTG | | |
| | CCCATTAACAGGTCTTCCAAAGTAC | | |
| | GAATGTCTACGTGTGTGCTTTGT | | |
| 133919_5536530_ | ATCTTCACCTGTATCACTGTCATTT | 120 | 384 |
| KP212157.1-HPV16- | TCGTTCTCGTCATCTGATATAGCAT | | |
| CNA138_1_16-REV | CCCCTGTTTTTTTTTCCACTACAG | | |
| | CCTCTACATAAAACCATCCATTACA | | |
| | TCCCGTACCCTCTTCCCCATT | | |
| 133919_5536530_ | TTAGAACCTGTACTGCATCTCTATG | 120 | 385 |
| KP212157.1-HPV16- | TTGTTTTGCTTCCTGTGCAGTAAAC | | |
| CNA138_1_18-REV | AACGCATGTGCTGTCTCTGTTTC | | |
| | TGCCTGTGTTAAATAATCATTATCA | | |
| | TTTACTATAAAATCTACCAAAT | | |
| 133919_5536530_ | AATCTCCTTTTTGCAGCTCTACTTT | 120 | 386 |
| KP212157.1-HPV16- | GTTTTTCTATACATATAGCTTTTAA | | |
| CNA138_1_20-REV | TCTAGGACTAATATTATTGTCTACA | | |
| | CATCCACTAATATCACTAAGTGGAC | | |
| | TACCCAAATACTTTCGTTTT | | |
| 133919_5536530_ | CCCACTTCCACCACTATACTGACTA | 120 | 387 |
| KP212157.1-HPV16- | CATGGTGTTTCAGTCTCATGGCGCC | | |
| CNA138_1_22-REV | CTTCTACCTGTAACATCTGCTGA | | |
| | GTTTCCACTTCAGTATTGCCATACC | | |
| | CGCTGTCTTCGCTTTCAAATAA | | |
| 133919_5536530_ | CCTTTGCATTACTAGTTTTTAGTAC | 120 | 388 |
| KP212157.1-HPV16- | ATTTAAAATATTTGTAAGTGGTGTT | | |
| CNA138_1_24-REV | TGGCATATAGTGTGTCTTTCACTA | | |
| | ACACCCTCTCCCCCACTTCCACTAC | | |
| | TGTACTGACTGCAACCACCCC | | |
| 133919_5536530_ | GTAAGTCCAAATGCAGCAATACACC | 120 | 389 |
| KP212157.1-HPV16- | AATCGCAACACGTTGATTTATTACT | | |
| CNA138_1_26-REV | TTTAAATGGTCTTACTAATTCTGA | | |
| | AAAACTCACCCCGTATAACTCTTTA | | |
| | AATTTTGCTAACATTGCTGCC | | |
| 133919_5536530_ | ACATTTATATCTTACTAATAGTAAC | 120 | 390 |
| KP212157.1-HPV16- | ACAACCATTCCCCATGAACATGCTA | | |
| CNA138_1_28-REV | AACTTTGAATGTGTAAATATAAAC | | |
| | AATATTGTTGTAATAGTGTTTTTAT | | |
| | ACTGTCAGCTATACTGGGTGT | | |
| 133919_5536530_ | TATACCAATATAATGCTGCTGCTGT | 120 | 391 |
| KP212157.1-HPV16- | ACTACGCAATTTTGGAGGCTCTATC | | |
| CNA138_1_30-REV | ATCATACACATTGGAGACACACA | | |
| | TAATAGTTTAGACAGCAATTTTTCA | | |
| | ATTGTTTCTCTATTTTTTCCAC | | |
| 133919_5536530_ | TGTACCATCTGTGATAATTCAAATG | 120 | 392 |
| KP212157.1-HPV16- | TACAATCATTAAAACTATGTTGTAA | | |
| CNA138_1_32-REV | TACTGTTTGTCTTTGTATCCATTCT | | |
| | GGCGTGTCTCCATACACTTCACTAA | | |
| | TATTTGATATACCTGTTTTA | | |
| 133919_5536530_ | AATTTTTGCCTGTGAATTACTTTTT | 120 | 393 |
| KP212157.1-HPV16- | AGAAAGGCACTTGCATTACTATTAG | | |
| CNA138_1_34-REV | TGTCTGCCAATTGTGCATATTTAT | | |
| | ATGCAATTTCACTATCGTCTACTAT | | |
| | GTCATTATCGTAGGCCCATTG | | |
| 133919_5536530_ | GCTTCCAATCACCTCCATCATCTAC | 120 | 394 |
| KP212157.1-HPV16- | CCTATCACATCTATATTTTATCCAT | | |
| CNA138_1_36-REV | TGACTCATACTCATTTGTTTTTTTT | | |
| | CTGCTCGTTTATAATGTCTACACAT | | |
| | TGTTGCACAATCCTTTACAA | | |
| 133919_5536530_ | TTAGCTGCACCATATAGTAATATGC | 120 | 395 |
| KP212157.1-HPV16- | AATTTTTTTTAGGTATGCCTTGCAA | | |
| CNA138_1_38-REV | AAATCTTTTTAATGCAGTTAAAAAT | | |
| | GACATAAACTCTACACCTTGATACC | | |
| | TTAAAAACATAACAATTTGC | | |
| 133919_5536530_ | ACCTATTTTGGCATCTGCTAATGGT | 120 | 396 |
| KP212157.1-HPV16- | TGTAACCAAAAATGGCTTTTAGAAT | | |
| CNA138_1_40-REV | TTACAAAACATATTACAGACCCTT | | |
| | GCAGAAATTTCATTAAACTCATACC | | |
| | AAATAATGATTTACCTGTGTT | | |
| 133919_5536530_ | ATTTTAGTTGTACCAATGGTCTATG | 120 | 397 |
| KP212157.1-HPV16- | CTTTACATCCATAGAAACTAAATTT | | |
| CNA138_1_42-REV | CCATCCAATGCATTTCTTAAATTGT | | |
| | CATCTATGTAGTTCCAACAGGGCAC | | |
| | TGTAGCATCATCTAACATAC | | |
| 133919_5536530_ | CCGTTTTCGTCAAATGGAAACTCAT | 120 | 398 |
| KP212157.1-HPV16- | TAGGAAATGTAAACACCACCAATCT | | |
| CNA138_1_44-REV | ATTATGTAAATAAGGCCACCTAG | | |
| | AATCTGTACCAGCATTAATGTTAGA | | |
| | TGTAATTAATAATGGAGGGCAT | | |
| 133919_5536530_ | CGTTGGCAAAGAGTCTCCATCGTTT | 120 | 399 |
| KP212157.1-HPV16- | TCCTTGTCCTCGTCCTCGTGCAAAC | | |
| CNA138_1_46-REV | TTAATCTGGACCACGTCCTTGA | | |
| | GAAAAAGGATTTCCAGTTCTTATCA | | |
| | TTAAGCTCATACACTGGATTTCC | | |
| 133919_5536530_ | GGCCTTGTAATAAATAGCACATTCT | 120 | 400 |
| KP212157.1-HPV16- | AGGCGCATGTGTTTCCAATAGTCTA | | |
| CNA138_1_48-REV | TATGGTCACGTAGGTCTGTACTA | | |
| | TCATTTTCATAATGTGTTAGTATTT | | |
| | TGTCCTGACACACATTTAAACG | | |
| 133919_5536530_ | GTGAGTTATATATTGTTTCTAACGT | 120 | 401 |
| KP212157.1-HPV16- | TAGTTGCAGTTCAATTGCTTGTAAT | | |
| CNA138_1_50-REV | GCTTTATTCTTTGATACAGCCAGT | | |
| | GTTGGCACCACCTGGTGGTTAATAT | | |
| | GTTTAAATCCCATTTCTCTGG | | |
| 133919_5536530_ | ATGTCTCCATCAAACTGCACTTCCA | 120 | 402 |
| KP212157.1-HPV16- | CTGTATATCCATGTTTTTTTATACA | | |
| CNA138_1_52-REV | TCCTGTTGGTGCAGTTAAATACAC | | |
| | TTCAAGGCTAACGTCTTGTAATGTC | | |
| | CACTTTTCATTACTATATTGT | | |
| 133919_5536530_ | TATTCCTTCATGAACATAATATAAA | 120 | 403 |
| KP212157.1-HPV16- | CCATAATAGTCAACTTGACCCTCTA | | |
| CNA138_1_54-REV | CCACAGTTACTGATGCTTCTTCAC | | |
| | AAATATATATATGTGTCCAGTTTGT | | |
| | ATAATGCATTGTATTGCATAT | | |
| 133919_5536530_ | CGTTGCTGCTAAACACAGATGTAGG | 120 | 404 |
| KP212157.1-HPV16- | ACATAATATTACCTGACCACCCGCA | | |
| CNA138_1_56-REV | TGAACTTCCCATACTTTATTTTT | | |
| | ACTATATTTTTCTGCATCATCTTTA | | |
| | AACTGCACAAAATATGTTCGTA | | |
| 133919_5536530_ | GATCTTGGTCGCTGGATAGTCGTCT | 120 | 405 |
| KP212157.1-HPV16- | GTGTTTCTTCGGTGCCCAAGGCGAC | | |
| CNA138_1_58-REV | GGCTTTGGTATGGGTCGCGGC | | |
| | GGGGTGGTTGGCCAAGTGCTGCCTA | | |
| | ATAATTTCAGGAGAGGATACTTCG | | |
| 133919_5536530_ | ATTACAGTTAATCCGTCCTTTGTGT | 120 | 406 |
| KP212157.1-HPV16- | GAGCTGTTAAATGCAGTGAGGATTG | | |
| | GAGCACTGTCCACTGAGTCTCT | | |
| CNA138_1_60-REV | GTGCAACAACTTAGTGGTGTGGCAG | | |
| | GGGTTTCCGGTGTCTGGCTCTGA | | |
| 133919_5536530_ | TCCAATGCCATGTAGACGACACTGC | 120 | 407 |
| KP212157.1-HPV16- | AGTATACAATGTACAATGCTTTTTA | | |
| CNA138_1_62-REV | AATCTATATCTTAAACATTTTAAA | | |
| | GTATTAGCATCACCTTTTAAATGTA | | |
| | CTATGGGTGTAGTGTTACTAT | | |
| 133919_5536530_ | CCAGTAGACACTGTAATAGTTTTTG | 120 | 408 |
| KP212157.1-HPV16- | GTATTTTAACTTGAGACAAAAATTG | | |
| CNA138_1_64-REV | GTCACGTTGCCATTCACTATCATA | | |
| | TGTAAGTGTAACAATTGCACTTTTA | | |
| | TGTTTTACATTATGTCCTGTC | | |
| 133919_5536530_ | CAGACAAAAGCAGCGGACGTATTAA | 120 | 409 |
| KP212157.1-HPV16- | TAGGCAGACACACAAAAGCACACAA | | |
| CNA138_1_66-REV | AAGCAAAGCAAAAAGCACGCCA | | |
| | GTAATGTTGTGGATGCAGTATCAAG | | |
| | ATTTGTCATATAGACATAAATCC | | |
| 133919_5536530_ | TGTATTAAAAATAATGGTATATAAA | 120 | 410 |
| KP212157.1-HPV16- | CAAATATAATATATACAATAAAACA | | |
| CNA138_1_68-REV | CCTAAACGCAGAGGCTGCTGTTAT | | |
| | CCACAATAGTAATACCAATATTATT | | |
| | AATGATGTGTATGTAGACACA | | |
| 133919_5536530_ | TAGTAAGTTATGGTATACAACAATT | 86 | 411 |
| KP212157.1-HPV16- | ATATGTAACAATTACATTATGTACA | | |
| CNA138_1_70-REV | TATACATTATGTAATTAAAAAGCGT | | |
| | GCATGTGTATG | | |
| 133919_5536531_ | GGTATAATGTCAGGTGGACATGTAC | 120 | 412 |
| KP212157.1-HPV16- | CTGCCTGTTTGCATGTTTTATAAAG | | |
| CNA138_2_2-REV | TTGGGTAGCCGATGCACGTTTT | | |
| | GTGCGTTTTGCAGAACGTTTGTGTC | | |
| | GCATTGTTAAGTGATAACAGTTT | | |
| 133919_5536531_ | GGAATATACCCAGTGCGTCCGCCTG | 120 | 413 |
| KP212157.1-HPV16- | TACCCGACCCCGTTCCAATTCCTAA | | |
| CNA138_2_4-REV | CCCACCAAAAAATACACCCATA | | |
| | CTTCCATATTGTAATATTTGATCAG | | |
| | CAATAGTTTTGCCTTCAACCTTA | | |
| 133919_5536531_ | AAACTAGTTTCTTCCACTAAAGAAA | 120 | 414 |
| KP212157.1-HPV16- | CTATAGAAGGATCAGAAGGGCCCAC | | |
| CNA138_2_6-REV | AGGATCTACTGTTAAAGGGGGT | | |
| | CTTACAGGAGCAAGTGTATCTGTAG | | |
| | CTGTGGGAGGCCTTGTTCCCAAT | | |
| 133919_5536532_ | GGAATTTCTTCATAATTATGTGTAC | 120 | 415 |
| KP212157.1-HPV16- | TAATAGTGGATGATGAAAGTGTAAA | | |
| CNA138_3_2-REV | ATGCCCTCCAGTTTCTGCAGGTG | | |
| | TTGGAGGCTGCAATACAGATGGGTC | | |
| | AGTGAAAGTGGGATTATTATGT | | |
| 133919_5536532_ | ACTTTAACTTGTTGTGTTGTGCGAC | 120 | 416 |
| KP212157.1-HPV16- | TATATAATCCTAGGCGTGCCACTGG | | |
| CNA138_3_4-REV | GCGAGACCCTGGTATGGGTGTG | | |
| | CTACTAGTTACTGTGTTAGGGTTTG | | |
| | TGCTAACAATAAATGTATCCATA | | |
| 133919_5536532_ | ATATTAATACTATTATCATTACTAG | 120 | 417 |
| KP212157.1-HPV16- | AAAAATATAATGTATTATCCACATC | | |
| CNA138_3_6-REV | TATACCTTCATATGCAGGATTATCA | | |
| | TATGTAATAAGTTTAGTGGGAGTGG | | |
| | TTACAAAAGCAGGGTCTACA | | |
| 133919_5536532_ | CCACTACGAGTACGTAGTGTTTGTT | 120 | 418 |
| KP212157.1-HPV16- | TATTACCAATTCTACTGTACCTAAT | | |
| CNA138_3_8-REV | GCCAGTACGCCTAGAGGTTAATG | | |
| | CTGGCCTATGTAAAGCAACTATATC | | |
| | CAAAAAGTCAGGATCTGGAGCT | | |
| 133919_5536532_ | GAGGCTGCATGTGAAGTGGTAGTAT | 120 | 419 |
| KP212157.1-HPV16- | ATGTAGAAGGTGTTATAGTTTGTAA | | |
| CNA138_3_10-REV | TTCTATTTCTTCTGCAGGATCAA | | |
| | TAGTACTAAAATCATAATAATAATG | | |
| | TACCTTAGCACCTATAGATTTT | | |
| 133919_5536532_ | TTTGCAGGAATATAACCTGATAAAG | 120 | 420 |
| KP212157.1-HPV16- | ATGTAGAGGGTACAGATGGTACCGG | | |
| CNA138_3_12-REV | GGTTGTAGAAGTATCTGTAATA | | |
| | AAGTCATCTGCATAAATATCATATA | | |
| | ATCCATTATTAATAGAAGTAGGT | | |
| 133919_5536532_ | TATTGTGGAGACCCTGGAACTATAG | 120 | 421 |
| KP212157.1-HPV16- | GAATTAATGAAGGAGCTTGGTCAGT | | |
| CNA138_3_14-REV | TATATTAATGGGTATATCAGGAC | | |
| | CTGATACTAAAGGAATATTGTATGC | | |
| | ACCACCAAAAGGAATTGTTGTA | | |
| 133919_5536532_ | TGGCCTCACTAGGCAGCCAAAGAGA | 120 | 422 |
| KP212157.1-HPV16- | CATCTGAAAAAAAATATGGTAAACG | | |
| CNA138_3_16-REV | TTTACGTCGTTTTCGTAACATGT | | |
| | AATAACTAGGATGTAAATAAAAGTC | | |
| | ACCTGCATCAGCAATAATTGTA | | |
| 133919_5536532_ | AATAGGGATGTCCAACTGCAAGTAG | 120 | 423 |
| KP212157.1-HPV16- | TCTGGATGTTCCTGCATGATAATAT | | |
| CNA138_3_18-REV | ATGTTTGTGCGTGCAACATATTC | | |
| | ATCCGTGCTTACAACCTTAGATACT | | |
| | GGGACAGGAGGCAAGTAGACAG | | |
| 133919_5536532_ | ATGAGGTGTCAGGAAAACCAAACTT | 120 | 424 |
| KP212157.1-HPV16- | ATTGGGGTCAGGTAAATGTATTCTA | | |
| CNA138_3_20-REV | AATACCCTGTATTGTAATCCTGA | | |
| | TACTTTAGGAACTAATATTTTGTTA | | |
| | TTGTTAGGTTTTTTAATAGGAA | | |
| 133919_5536532_ | CTGTGTCATCCAATTTATTTAATAA | 120 | 425 |
| KP212157.1-HPV16- | AGGATGGCCACTAATGCCCACACCT | | |
| CNA138_3_22-REV | AATGGCTGACCACGACCTACCTC | | |
| | AACACCTACACAGGCCCAAACCAGC | | |
| | CGCTGTGTATCTGGATTATAAA | | |
| 133919_5536532_ | CCCAGTGTTCCCCTATAGGTGGTTT | 120 | 426 |
| KP212157.1-HPV16- | GCAACCAATTAAACACAATTGTGTT | | |
| CNA138_3_24-REV | TGTTTGTAATCCATAGATATACAT | | |
| | TCTCTATTATCCACACCTGCATTTG | | |
| | CTGCATAAGCACTAGCATTTT | | |
| 133919_5536532_ | AGTCCATAGCACCAAAGCCAGTATC | 120 | 427 |
| KP212157.1-HPV16- | AACCATATCACCATCCTGAATAACT | | |
| CNA138_3_26-REV | GTGTTTATTAACTCTAATGGTGG | | |
| | ACAATCACCTGGATTTACTGCAACA | | |
| | TTGGTACATGGGGATCCTTTGC | | |
| 133919_5536532_ | AATAAAAAAATAAGCTGTCGCCATA | 120 | 428 |
| KP212157.1-HPV16- | TGGTTCTGACACCATTTTAATATAA | | |
| CNA138_3_28-REV | TCTGGATATTTGCAAATAGATGTA | | |
| | CAAATATCCAGTGGAACTTCACTTT | | |
| | TGTTAGCCTGTAATGTAGTAA | | |
| 133919_5536532_ | TGGAACTGGCTAAATTTGCAGTAGA | 120 | 429 |
| KP212157.1-HPV16- | CCCAGAGCCTTTAATGTATAAATCG | | |
| CNA138_3_30-REV | TCTGGTACATTTTCACCAACAGT | | |
| | ACCAGCCCTATTAAATAAATGTCTA | | |
| | ACAAACATTTGTTCCCTTCGTA | | |
| 133919_5536532_ | ATAGTTGGTTACCCCAACAAATGCC | 120 | 430 |
| KP212157.1-HPV16- | ATTATTGTGGCCCTGTGCTCGTTGT | | |
| CNA138_3_32-REV | AACCAATAAGGTTTATTGAATAT | | |
| | TTGGGCATCAGAGGTAACCATAGAA | | |
| | CCACTAGGTGTAGGAAAATAAT | | |
| 133919_5536532_ | ATTCCTCCCCATGTCGTAGGTACTC | 120 | 431 |
| KP212157.1-HPV16- | CTTAAAGTTAGTATTTTTATATGTA | | |
| CNA138_3_34-REV | GTTTCTGAAGTAGATATGGCAGCA | | |
| | CATAATGACATATTTGTACTGCGTG | | |
| | TAGTATCAACAACAGTAACAA | | |
| 133919_5536532_ | GGGGAGGTTGTAGACCAAAATTCCA | 120 | 432 |
| KP212157.1-HPV16- | GTCCTCCAAAATAGTGGAATTCATA | | |
| CNA138_3_36-REV | GAATGTATGTATGTCATAACGTC | | |
| | TGCAGTTAAGGTTATTTTGCACAGT | | |
| | TGAAAAATAAACTGTAAATCAT | | |
| 133919_5536532_ | TTACTTCCCAAAAAGTGTATTTTTT | 120 | 433 |
| KP212157.1-HPV16- | AAGGGGATCTTCTTTAGGTGCTGGA | | |
| CNA138_3_38-REV | GGTGTATGTTTTTGACAAGCAATT | | |
| | GCCTGGGATGTTACAAACCTATAAG | | |
| | TATCTTCTAGTGTGCCTCCTG | | |
| 133919_5536532_ | TGGGTGTAGCTTTTCGTTTTCCTAA | 120 | 434 |
| KP212157.1-HPV16- | TGTAAATTTTGGTTTGGCCTTCAAT | | |
| CNA138_3_40-REV | CCTGCTTGTAGTAAAAATTTGCGT | | |
| | CCTAAAGGAAACTGATCTAGGTCTG | | |
| | CAGAAAACTTTTCCTTTAAAT | | |
| 133919_5536533_ | GATGTAGCAAATATAGTTTATATAC | 114 | 435 |
| KP212157.1-HPV16- | AATGAATAACCACAACACAATTAGT | | |
| CNA138_4_2-REV | AGGTGTTGAAACAATAAGTTTATT | | |
| | TATGTTGCATGACACAATAGTTACA | | |
| | CAAGCATTTAAAAAC | | |
| 133919_5536533_ | CTATTTAAAAAAACACATTTTGTGC | 120 | 436 |
| KP212157.1-HPV16- | CAAAAAGCTTGCAACCGAATTCGGT | | |
| CNA138_4_4-REV | TGAAGCTACAAAATGGCCGCTGG | | |
| | CGCTACAAAATATAGTATATATAAA | | |
| | ACAAAAACAGGATGTAGCAAAT | | |
| 133919_5536533_ | AAAAAACAATGGAATGGTTGGCAAG | 120 | 437 |
| KP212157.1-HPV16- | CAGTGCAGGTCAGGAAAACAGGGAT | | |
| CNA138_4_6-REV | TTGGCACGCATGGCAAGCAGG | | |
| | AAACGTACAAGTTTAAACCATAGTT | | |
| | GCTGACATAGAACTATTTAAAAAA | | |
| 133919_5536533_ | AGCCAAAAATATGTGCCTAACAGCG | 120 | 438 |
| KP212157.1-HPV16- | GTATGTAAGGCGTTGGCGCATAGTG | | |
| CNA138_4_8-REV | ATTTATTTTATATGACACAATGT | | |
| | ACATAGTGATTCAGTAGTTGCACAT | | |
| | AGTGCAGTGTAAAAAACAATGG | | |
| 133919_5536533_ | CAATGTATGACTAACCTTTACACAG | 120 | 439 |
| KP212157.1-HPV16- | TTCATGTATAAACTAGGGTGACATT | | |
| CNA138_4_10-REV | TAGTTGGCCTTAGAAGTTTAAAC | | |
| | CTTATGCCAAATATGCAATTAGGTT | | |
| | AGTTAAAACAAGCCAAAAATAT | | |

| HPV18_DoubleStrand_ | | | SEQ |
|---|---|---|---|
| SequenceName | Sequence | Length | ID#: |
| 625339_25353837_60975_ | CCTCAAAGCGCGCCATAGTATTGTG | 120 | 440 |
| HPV18REF.1_1_1 | GTGTGTTTCTCACATCTTTTATATA | | |
| | CACCGTTTTCGGTCCCGACCGTTTT | | |
| | CGGTTACTC | | |
| | CCTTTTTTATATACTACAATTGTTA | | |
| | AAAGTATTAAT | | |
| 625339_25353837_60975_ | GAAAACGGTGTATATAAAAGATGTG | 120 | 441 |
| HPV18REF.1_1_2 | AGAAACACACCACAATACTATGGCG | | |
| | CGCTTTGAGGATCCAACACGGCGAC | | |
| | CCTACAAG | | |
| | CTACCTGATCTGTGCACGGAACTGA | | |
| | ACACTTCACTGC | | |
| 625339_25353837_60975_ | CAAATACCTCTGTAAGTTCCAATAC | 120 | 442 |
| HPV18REF.1_1_3 | TGTCTTGCAATATACACAGGTTATT | | |
| | TCTATGTCTTGCAGTGAAGTGTTCA | | |
| | GTTCCGTGCA | | |
| | CAGATCAGGTAGCTTGTAGGGTCGC | | |
| | CGTGTTGGAT | | |
| 625339_25353837_60975_ | AAGACATAGAAATAACCTGTGTATA | 120 | 443 |
| HPV18REF.1_1_4 | TTGCAAGACAGTATTGGAACTTACA | | |
| | GAGGTATTTGAATTTGCATTTAAAG | | |
| | ATTTATTTGT | | |
| | GGTGTATAGAGACAGTATACCGCAT | | |
| | GCTGCATGCC | | |
| 625339_25353837_60975_ | ACACAGAGTCTGAATAATGTCTTAA | 120 | 444 |
| HPV18REF.1_1_5 | TTCTCTAATTCTAGAATAAAAATCT | | |
| | ATACATTTATGGCATGCAGCATGCG | | |
| | GTATACTGTC | | |
| | TCTATACACCACAAATAAATCTTTA | | |
| | AATGCAAATT | | |
| 625339_25353837_60975_ | ATAAATGTATAGATTTTTATTCTAG | 120 | 445 |
| HPV18REF.1_1_6 | AATTAGAGAATTAAGACATTATTCA | | |
| | GACTCTGTGTATGGAGACACATTGG | | |
| | AAAAACTAACT | | |
| | AACACTGGGTTATACAATTTATTAA | | |
| | TAAGGTGCC | | |
| 625339_25353837_60975_ | GTCGTTTTTCATTAAGGTGTCTAAG | 120 | 446 |
| HPV18REF.1_1_7 | TTTTTCTGCTGGATTCAACGGTTTC | | |
| | TGGCACCGCAGGCACCTTATTAATA | | |
| | AATTGTATAA | | |
| | CCCAGTGTTAGTTAGTTTTTCCAAT | | |
| | GTGTCTCCAT | | |
| 625339_25353837_60975_ | TGCGGTGCCAGAAACCGTTGAATCC | 120 | 447 |
| HPV18REF.1_1_8 | AGCAGAAAAACTTAGACACCTTAAT | | |
| | GAAAAACGACGATTTCACAACATAG | | |
| | CTGGGCACT | | |
| | ATAGAGGCCAGTGCCATTCGTGCTG | | |
| | CAACCGAGCAC | | |
| 625339_25353837_60975_ | GGTCCATGCATACTTAATATTATAC | 120 | 448 |
| HPV18REF.1_1_9 | TTGTGTTTCTCTGCGTCGTTGGAGT | | |
| | CGTTCCTGTCGTGCTCGGTTGCAGC | | |
| | ACGAATGG | | |
| | CACTGGCCTCTATAGTGCCCAGCTA | | |
| | TGTTGTGAAATC | | |
| 625339_25353837_60975_ | GACAGGAACGACTCCAACGACGCAG | 120 | 449 |
| HPV18REF.1_1_10 | AGAAACACAAGTATAATATTAAGTA | | |
| | TGCATGGACCTAAGGCAACATTGCA | | |
| | AGACATTGT | | |
| | ATTGCATTTAGAGCCCCAAAATGAA | | |
| | ATTCCGGTTGA | | |
| 625339_25353837_60975_ | ACTCCATCTATTTCATCGTTTTCTT | 120 | 450 |
| HPV18REF.1_1_11 | CCTCTGAGTCGCTTAATTGCTCGTG | | |
| | ACATAGAAGGTCAACCGGAATTTCA | | |
| | TTTTGGGGCT | | |
| | CTAAATGCAATACAATGTCTTGCAA | | |
| | TGTTGCCTTA | | |
| 625339_25353837_60975_ | CCTTCTATGTCACGAGCAATTAAGC | 120 | 451 |
| HPV18REF.1_1_12 | GACTCAGAGGAAGAAAACGATGAAA | | |
| | TAGATGGAGTTAATCATCAACATTT | | |
| | ACCAGCCCG | | |
| | ACGAGCCGAACCACAACGTCACACA | | |
| | ATGTTGTGTAT | | |
| 625339_25353837_60975_ | CGAAGGTCGTCTGCTGAGCTTTCTA | 120 | 452 |
| HPV18REF.1_1_13 | CTACTAGCTCAATTCTGGCTTCACA | | |
| | CTTACAACACATACACAACATTGTG | | |
| | TGACGTTGT | | |
| | GGTTCGGCTCGTCGGGCTGGTAAAT | | |
| | GTTGATGATTA | | |
| 625339_25353837_60975_ | GTGTTGTAAGTGTGAAGCCAGAATT | 120 | 453 |
| HPV18REF.1_1_14 | GAGCTAGTAGTAGAAAGCTCAGCAG | | |
| | ACGACCTTCGAGCATTCCAGCAGCT | | |
| | GTTTCTGA | | |
| | ACACCCTGTCCTTTGTGTGTCCGTG | | |
| | GTGTGCATCCCA | | |
| 625339_25353837_60975_ | CCGTTACAACCCGTGCCCTCCCCGT | 120 | 454 |
| HPV18REF.1_1_15 | CTGTACCTTCTGGATCAGCCATTGT | | |
| | TGCTTACTGCTGGGATGCACACCAC | | |
| | GGACACAC | | |
| | AAAGGACAGGGTGTTCAGAAACAGC | | |
| | TGCTGGAATGCT | | |
| 625339_25353837_60975_ | GCAGTAAGCAACAATGGCTGATCCA | 120 | 455 |
| HPV18REF.1_1_16 | GAAGGTACAGACGGGGAGGGCACGG | | |
| | GTTGTAACGGCTGGTTTTATGTACA | | |
| | AGCTATT | | |
| | GTAGACAAAAAAACAGGAGATGTAA | | |
| | TATCAGATGACGA | | |
| 625339_25353837_60975_ | GTTCCTTGTGTATCAATAAAATCTA | 120 | 456 |
| HPV18REF.1_1_17 | CCATATCCGACCCTGTGTCTGTTGC | | |
| | ATTTTCGTCCTCGTCATCTGATATT | | |
| | ACATCTCCTG | | |
| | TTTTTTTGTCTACAATAGCTTGTAC | | |
| | ATAAAACCAG | | |
| 625339_25353837_60975_ | GGACGAAAATGCAACAGACACAGGG | 120 | 457 |
| HPV18REF.1_1_18 | TCGGATATGGTAGATTTTATTGATA | | |
| | CACAAGGAACATTTTGTGAACAGGC | | |
| | AGAGCTAG | | |
| | AGACAGCACAGGCATTGTTCCATGC | | |
| | GCAGGAGGTCCA | | |
| 625339_25353837_60975_ | TTTTCTGTGCTGCCTCCTGCAAACT | 120 | 458 |
| HPV18REF.1_1_19 | TTCGTTTTAAAACATGCAACACTTG | | |
| | TGCATCATTGTGGACCTCCTGCGCA | | |
| | TGGAACAAT | | |
| | GCCTGTGCTGTCTCTAGCTCTGCCT | | |
| | GTTCACAAAAT | | |
| 625339_25353837_60975_ | CAATGATGCACAAGTGTTGCATGTT | 120 | 459 |
| HPV18REF.1_1_20 | TTAAAACGAAAGTTTGCAGGAGGCA | | |
| | GCACAGAAAACAGTCCATTAGGGGA | | |
| | GCGGCTGG | | |
| | AGGTGGATACAGAGTTAAGTCCACG | | |
| | GTTACAAGAAAT | | |
| 625339_25353837_60975_ | CCACTATCTGATATTGTAAACAGCC | 120 | 460 |
| HPV18REF.1_1_21 | GCCTTTTTGCCTTTTTCTGCCCACT | | |
| | ATTTAAAGATATTTCTTGTAACCGT | | |
| | GGACTTAACT | | |
| | CTGTATCCACCTCCAGCCGCTCCCC | | |
| | TAATGGACTG | | |
| 625339_25353837_60975_ | ATCTTTAAATAGTGGGCAGAAAAAG | 120 | 461 |
| HPV18REF.1_1_22 | GCAAAAAGGCGGCTGTTTACAATAT | | |
| | CAGATAGTGGCTATGGCTGTTCTGA | | |
| | AGTGGAAG | | |
| | CAACACAGATTCAGGTAACTACAAA | | |
| | TGGCGAACATGG | | |
| 625339_25353837_60975_ | TTGCCCTCTGTGCCCCCGTTGTCTA | 120 | 462 |
| HPV18REF.1_1_23 | TAGCCTCCGTACTGCCGCCACTACA | | |
| | TACATTGCCGCCATGTTCGCCATTT | | |
| | GTAGTTACC | | |
| | TGAATCTGTGTTGCTTCCACTTCAG | | |
| | AACAGCCATAG | | |
| 625339_25353837_60975_ | CGGCAATGTATGTAGTGGCGGCAGT | 120 | 463 |
| HPV18REF.1_1_24 | ACGGAGGCTATAGACAACGGGGGCA | | |
| | CAGAGGGCAACAACAGCAGTGTAGA | | |
| | CGGTAC | | |
| | AAGTGACAATAGCAATATAGAAAAT | | |
| | GTAAATCCACAATG | | |
| 62533925353837_60975_ | GCTAACATAGCTCCTTGTTTATTGT | 120 | 464 |
| HPV18REF.1_1_25 | TTACTTTTAACAAGTCTTTTAATTG | | |
| | TGCTATGGTACATTGTGGATTTACA | | |
| | TTTTCTATATT | | |
| | GCTATTGTCACTTGTACCGTCTACA | | |
| | CTGCTGTTG | | |
| 625339_25353837_60975_ | TACCATAGCACAATTAAAAGACTTG | 120 | 465 |
| HPV18REF.1_1_26 | TTAAAAGTAAACAATAAACAAGGAG | | |
| | CTATGTTAGCAGTATTTAAAGACAC | | |
| | ATATGGGCTA | | |
| | TCATTTACAGATTTAGTTAGAAATT | | |
| | TTAAAAGTGA | | |
| 625339_25353837_60975_ | TCTGCTATTGTTGGGTTTACTCCAA | 120 | 466 |
| HPV18REF.1_1_27 | ATATAGCTGTAACCCAATCTGTACA | | |
| | CGTGGTTTTATCACTTTTAAAATTT | | |
| | CTAACTAAATC | | |
| | TGTAAATGATAGCCCATATGTGTCT | | |
| | TTAAATACT | | |
| 625339_25353837_60975_ | TAAAACCACGTGTACAGATTGGGTT | 120 | 467 |
| HPV18REF.1_1_28 | ACAGCTATATTTGGAGTAAACCCAA | | |
| | CAATAGCAGAAGGATTTAAAACACT | | |
| | AATACAGCC | | |
| | ATTTATATTATATGCCCATATTCAA | | |
| | TGTCTAGACTG | | |
| 625339_25353837_60975_ | GTTAGTCTACTCTTACCACATTTGT | 120 | 468 |
| HPV18REF.1_1_29 | AACGCAACAGGGCTAATATTAATAC | | |
| | TCCCCATTTACAGTCTAGACATTGA | | |
| | ATATGGGCAT | | |
| | ATAATATAAATGGCTGTATTAGTGT | | |
| | TTTAAATCCT | | |
| 625339_25353837_60975_ | TAAATGGGGAGTATTAATATTAGCC | 120 | 469 |
| HPV18REF.1_1_30 | CTGTTGCGTTACAAATGTGGTAAGA | | |
| | GTAGACTAACAGTTGCTAAAGGTTT | | |
| | AAGTACGTT | | |
| | GTTACACGTACCTGAAACTTGTATG | | |
| | TTAATTCAACC | | |
| 625339_25353837_60975_ | ATATTTGATATTCCTGTTCTATACC | 120 | 470 |
| HPV18REF.1_1_31 | AATATAGTGCTGCAACACTACTTCG | | |
| | CAATTTTGGTGGTTGAATTAACATA | | |
| | CAAGTTTCAG | | |
| | GTACGTGTAACAACGTACTTAAACC | | |
| | TTTAGCAACT | | |
| 625339_25353837_60975_ | ACCAAAATTGCGAAGTAGTGTTGCA | 120 | 471 |
| HPV18REF.1_1_32 | GCACTATATTGGTATAGAACAGGAA | | |
| | TATCAAATATTAGTGAAGTAATGGG | | |
| | AGACACACC | | |
| | TGAGTGGATACAAAGACTTACTATT | | |
| | ATACAACATGG | | |
| 625339_25353837_60975_ | AGCTCATTATCAAATGCCCATTGTA | 120 | 472 |
| HPV18REF.1_1_33 | CCATTTCTGACAAATCAAAATTGCT | | |
| | ATCATCTATTCCATGTTGTATAATA | | |
| | GTAAGTCTTTG | | |
| | TATCCACTCAGGTGTGTCTCCCATT | | |
| | ACTTCACTA | | |
| 625339_25353837_60975_ | AATAGATGATAGCAATTTTGATTTG | 120 | 473 |
| HPV18REF.1_1_34 | TCAGAAATGGTACAATGGGCATTTG | | |
| | ATAATGAGCTGACAGATGAAAGCGA | | |
| | TATGGCATT | | |
| | TGAATATGCCTTATTAGCAGACAGC | | |
| | AACAGCAATGC | | |
| 625339_25353837_60975_ | CACATTGTGGCACAATCTTTTAAAT | 120 | 474 |
| HPV18REF.1_1_35 | ATTTAGCTTGGCAATTGCTTTTTAA | | |
| | AAAGGCAGCTGCATTGCTGTTGCTG | | |
| | TCTGCTAATA | | |
| | AGGCATATTCAAATGCCATATCGCT | | |
| | TTCATCTGTC | | |
| 625339_25353837_60975_ | AGCTGCCTTTTTAAAAAGCAATTGC | 120 | 475 |
| HPV18REF.1_1_36 | CAAGCTAAATATTTAAAAGATTGTG | | |
| | CCACAATGTGCAAACATTATAGGCG | | |
| | AGCCCAAAAA | | |
| | CGACAAATGAATATGTCACAGTGGA | | |
| | TACGATTTAG | | |
| 625339_25353837_60975_ | TGGTATCGCAGGAATTGCACTATTG | 120 | 476 |
| HPV18REF.1_1_37 | GTCTCCAATCTCCCCCTTCATCTAT | | |
| | TTTTGAACATCTAAATCGTATCCAC | | |
| | TGTGACATAT | | |
| | TCATTTGTCGTTTTTGGGCTCGCCT | | |
| | ATAATGTTTG | | |
| 625339_25353837_60975_ | ATGTTCAAAAATAGATGAAGGGGGA | 120 | 477 |
| HPV18REF.1_1_38 | GATTGGAGACCAATAGTGCAATTCC | | |
| | TGCGATACCAACAAATAGAGTTTAT | | |
| | AACATTTTT | | |
| | AGGAGCCTTAAAATCATTTTTAAAA | | |
| | GGAACCCCCAA | | |
| 625339_25353837_60975_ | ClCATTCCAAAATATGATTTTCCTG | 120 | 478 |
| HPV18REF.1_1_39 | TATTTGCTGGTCCACAAAATACTAA | | |
| | ACAATTTTTTTTGGGGGTTCCTTTT | | |
| | AAAAATGATTT | | |
| | TAAGGCTCCTAAAAATGTTATAAAC | | |
| | TCTATTTGT | | |
| 625339_25353837_60975_ | AAAAAATTGTTTAGTATTTTGTGGA | 120 | 479 |
| HPV18REF.1_1_40 | CCAGCAAATACAGGAAAATCATATT | | |
| | TTGGAATGAGTTTTATACACTTTAT | | |
| | ACAAGGAGCA | | |
| | GTAATATCATTTGTGAATTCCACTA | | |
| | GTCATTTTTG | | |
| 625339_25353837_60975_ | CAACACGTGGTCGTTGCATCATCTA | 120 | 480 |
| HPV18REF.1_1_41 | ACATGGCCACCTTAGTATCTGTTAA | | |
| | CGGTTCCAACCAAAAATGACTAGTG | | |
| | GAATTCACA | | |
| | AATGATATTACTGCTCCTTGTATAA | | |
| | AGTGTATAAAA | | |
| 625339_25353837_60975_ | GTTGGAACCGTTAACAGATACTAAG | 120 | 481 |
| HPV18REF.1_1_42 | GTGGCCATGTTAGATGATGCAACGA | | |
| | CCACGTGTTGGACATACTTTGATAC | | |
| | CTATATGAG | | |
| | AAATGCGTTAGATGGCAATCCAATA | | |
| | AGTATTGATAG | | |
| 625339_25353837_60975_ | TGTATATTTGTGGTTAGTAGTATTG | 120 | 482 |
| HPV18REF.1_1_43 | GAGGACATTTTAGTTGTATTAATGG | | |
| | TTTGTGCTTTCTATCAATACTTATT | | |
| | GGATTGCCAT | | |
| | CTAACGCATTTCTCATATAGGTATC | | |
| | AAAGTATGTC | | |
| 625339_25353837_60975_ | AAAGCACAAACCATTAATACAACTA | 120 | 483 |
| HPV18REF.1_1_44 | AAATGTCCTCCAATACTACTAACCA | | |
| | CAAATATACATCCAGCAAAGGATAA | | |
| | TAGATGGCCA | | |
| | TATTTAGAAAGTAGAATAACAGTAT | | |
| | TTGAATTTCC | | |
| 625339_25353837_60975_ | CAATTTTTGTCATTTATTTCATATA | 120 | 484 |
| HPV18REF.1_1_45 | CTGGATTGCCATTTTTATCAAATGG | | |
| | AAATGCATTTGGAAATTCAAATACT | | |
| | GTTATTCTACTT | | |
| | TCTAAATATGGCCATCTATTATCCT | | |
| | TTGCTGGA | | |
| 625339_25353837_60975_ | AAATGCATTTCCATTTGATAAAAAT | 120 | 485 |
| HPV18REF.1_1_46 | GGCAATCCAGTATATGAAATAAATG | | |
| | ACAAAAATTGGAAATGTTTTTTTGA | | |
| | AAGGACATGG | | |
| | TCCAGATTAGATTTGCACGAGGAAG | | |
| | AGGAAGATGC | | |
| 625339_25353837_60975_ | GGTCTATGATTTTGTCCTGCAACGC | 120 | 486 |
| HPV18REF.1_1_47 | ACTTAAACGTTCCGAAAGGGTTTCC | | |
| | TTCGGTGTCTGCATCTTCCTCTTCC | | |
| | TCGTGCAAA | | |
| | TCTAATCTGGACCATGTCCTTTCAA | | |
| | AAAAACATTTC | | |
| 625339_25353837_60975_ | AGACACCGAAGGAAACCCTTTCGGA | 120 | 487 |
| HPV18REF.1_1_48 | ACGTTTAAGTGCGTTGCAGGACAAA | | |
| | ATCATAGACCACTATGAAAATGACA | | |
| | GTAAAGACA | | |
| | TAGACAGCCAAATACAGTATTGGCA | | |
| | ACTAATACGTT | | |
| 625339_25353837_60975_ | CCTGGTGGTTTAATGTCTGTATGCC | 120 | 488 |
| HPV18REF.1_1_49 | ATGTTCCCTTGCTGCAAAGAATATT | | |
| | GCATTTTCCCAACGTATTAGTTGCC | | |
| | AATACTGTAT | | |
| | TTGGCTGTCTATGTCTTTACTGTCA | | |
| | TTTTCATAGT | | |
| 625339_25353837_60975_ | GGGAAAATGCAATATTCTTTGCAGC | 120 | 489 |
| HPV18REF.1_1_50 | AAGGGAACATGGCATACAGACATTA | | |
| | AACCACCAGGTGGTGCCAGCCTATA | | |
| | ACATTTCAA | | |
| | AAAGTAAAGCACATAAAGCTATTGA | | |
| | ACTGCAAATGG | | |
| 625339_25353837_60975_ | ATGTGTCTTGCAGTGTCCAATCCTC | 120 | 490 |
| HPV18REF.1_1_51 | GGTTTTGTATGCACTTTGTGCAAGG | | |
| | CCTTGTAGGGCCATTTGCAGTTCAA | | |
| | TAGCTTTAT | | |
| | GTGCTTTACTTTTTGAAATGTTATA | | |
| | GGCTGGCACCA | | |
| 625339_25353837_60975_ | CCCTACAAGGCCTTGCACAAAGTGC | 120 | 491 |
| HPV18REF.1_1_52 | ATACAAAACCGAGGATTGGACACTG | | |
| | CAAGACACATGCGAGGAACTATGGA | | |
| | ATACAGAA | | |
| | CCTACTCACTGCTTTAAAAAAGGTG | | |
| | GCCAAACAGTAC | | |
| 625339_25353837_60975_ | ACACACTGTCCCATGCTACATAGGT | 120 | 492 |
| HPV18REF.1_1_53 | CATACAATTGTCTTTGTTGCCATCA | | |
| | AAATATACTTGTACTGTTTGGCCAC | | |
| | CTTTTTTAAA | | |
| | GCAGTGAGTAGGTTCTGTATTCCAT | | |
| | AGTTCCTCGC | | |
| 625339_25353837_60975_ | AAGTATATTTTGATGGCAACAAAGA | 120 | 493 |
| HPV18REF.1_1_54 | CAATTGTATGACCTATGTAGCATGG | | |
| | GACAGTGTGTATTATATGACTGATG | | |
| | CAGGAACAT | | |
| | GGGACAAAACGGCTACCTGTGTAAG | | |
| | TCACAGGGGAT | | |
| 625339_25353837_60975_ | TTTCACATTCACTTTTAAATTCTAT | 120 | 494 |
| HPV18REF.1_1_55 | ATAAAACGTGTTGTACCCTTCCTTT | | |
| | ACATAATACAATCCCCTGTGACTTA | | |
| | CACAGGTAGCC | | |
| | GTTTTGTCCCATGTTCCTGCATCAG | | |
| | TCATATAAT | | |
| 625339_25353837_60975_ | TGTATTATGTAAAGGAAGGGTACAA | 120 | 495 |
| HPV18REF.1_1_56 | CACGTTTTATATAGAATTTAAAAGT | | |
| | GAATGTGAAAAATATGGGAACACAG | | |
| | GTACGTGGG | | |
| | AAGTACATTTTGGGAATAATGTAAT | | |
| | TGATTGTAATG | | |
| 625339_25353837_60975_ | GTAGCTGTTTAACAAGCTGAGTAGC | 120 | 496 |
| HPV18REF.1_1_57 | GGATACCGTGTCGTCACTGGTACTG | | |
| | CACATAGAGTCATTACAATCAATTA | | |
| | CATTATTCC | | |
| | CAAAATGTACTTCCCACGTACCTGT | | |
| | GTTCCCATATT | | |
| 625339_25353837_60975_ | ACTCTATGTGCAGTACCAGTGACGA | 120 | 497 |
| HPV18REF.1_1_58 | CACGGTATCCGCTACTCAGCTTGTT | | |
| | AAACAGCTACAGCACACCCCCTCAC | | |
| | CGTATTCCA | | |
| | GCACCGTGTCCGTGGGCACCGCAAA | | |
| | GACCTACGGCC | | |
| 625339_25353837_60975_ | GTCCACAATGCTGCTTCTCCGCGAG | 120 | 498 |
| HPV18REF.1_1_59 | TCCACAGTGTCCAGGTCGTGTAGCA | | |
| | GCCGACGTCTGGCCGTAGGTCTTTG | | |
| | CGGTGCC | | |
| | CACGGACACGGTGCTGGAATACGGT | | |
| | GAGGGGGTGTGCT | | |
| 625339_25353837_60975_ | AGACGTCGGCTGCTACACGACCTGG | 120 | 499 |
| HPV18REF.1_1_60 | ACACTGTGGACTCGCGGAGAAGCAG | | |
| | CATTGTGGACCTGTCAACCCACTTC | | |
| | TCGGTGC | | |
| | AGCTACACCTACAGGCAACAACAAA | | |
| | AGACGGAAACTCT | | |
| 625339_25353837_60975_ | AACATTTTAAACTGTTTCTGTCACC | 120 | 500 |
| HPV18REF.1_1_61 | TTTTAAATGTATTATAGGCGTAGTG | | |
| | TTACCACTACAGAGTTTCCGTCTTT | | |
| | TGTTGTTGCC | | |
| | TGTAGGTGTAGCTGCACCGAGAAGT | | |
| | GGGTTGACAG | | |
| 625339_25353837_60975_ | GTAGTGGTAACACTACGCCTATAAT | 120 | 501 |
| HPV18REF.1_1_62 | ACATTTAAAAGGTGACAGAAACAGT | | |
| | TTAAAATGTTTACGGTACAGATTGC | | |
| | GAAAACATA | | |
| | GCGACCACTATAGAGATATATCATC | | |
| | CACCTGGCATT | | |
| 625339_25353837_60975_ | GTGTTTCACTATGGTATGTTACAGT | 120 | 502 |
| HPV18REF.1_1_63 | CAGTATTCCTGTTTTTTCATTGCCT | | |
| | GCACCTGTCCAATGCCAGGTGGATG | | |
| | ATATATCTCT | | |
| | ATAGTGGTCGCTATGTTTTCGCAAT | | |
| | CTGTACCGTA | | |
| 625339_25353837_60975_ | GGACAGGTGCAGGCAATGAAAAAAC | 120 | 503 |
| HPV18REF.1_1_64 | AGGAATACTGACTGTAACATACCAT | | |
| | AGTGAAACACAAAGAACAAAATTTT | | |
| | TAAATACTG | | |
| | TTGCAATTCCAGATAGTGTACAAAT | | |
| | ATTGGTGGGAT | | |
| 625339_25353837_60975_ | AAAATAAAAAAATAAGTGATAACAT | 120 | 504 |
| HPV18REF.1_1_65 | ATTGGTACTACAGCATATGTATTAC | | |
| | ATTGTCATGTATCCCACCAATATTT | | |
| | GTACACTATCT | | |
| | GGAATTGCAACAGTATTTAAAAATT | | |
| | TTGTTCTTT | | |
| 625339_25353837_60975_ | ACATGACAATGTAATACATATGCTG | 120 | 505 |
| HPV18REF.1_1_66 | TAGTACCAATATGTTATCACTTATT | | |
| | TTTTTATTTTGCTTTTGTGTATGCA | | |
| | TGTATGTGTGC | | |
| | TGCCATGTCCCGCTTTTGCCATCTG | | |
| | TCTGTATGT | | |
| 625339_25353837_60975_ | CTGTGGCAGGGGACGTTATTACCAC | 120 | 506 |
| HPV18REF.1_1_67 | AATATACACAAATACCAATACCCAT | | |
| | GCATACGCACACATACAGACAGATG | | |
| | GCAAAAGC | | |
| | GGGACATGGCAGCACACATACATGC | | |
| | ATACACAAAAGC | | |
| 625339_25353837_60975_ | GTGCGTATGCATGGGTATTGGTATT | 120 | 507 |
| HPV18REF.1_1_68 | TGTGTATATTGTGGTAATAACGTCC | | |
| | CCTGCCACAGCATTCACAGTATATG | | |
| | TATTTTGTT | | |
| | TTTTATTGCCCATGTTACTATTGCA | | |
| | TATACATGCTA | | |
| 625339_25353837_60975_ | AAATATACAATGTACAATACACTGT | 120 | 508 |
| HPV18REF.1_1_69 | ATAAAACAACCTATACAATTACTGT | | |
| | AAAGACAATATAGCATGTATATGCA | | |
| | ATAGTAACATG | | |
| | GGCAATAAAAAACAAAATACATATA | | |
| | CTGTGAATG | | |
| 625339_25353837_60975_ | TATTGTCTTTACAGTAATTGTATAG | 120 | 509 |
| HPV18REF.1_1_70 | GTTGTTTTATACAGTGTATTGTACA | | |
| | TTGTATATTTTGTTTTATACCTTTT | | |
| | ATGCTTTTTGTA | | |
| | TTTTTGTAATAAAAGTATGGTATCC | | |
| | CACCGTGC | | |
| 625339_25353837_60975_ | GTACCAGATTGTTTACATGTTTTAT | 120 | 510 |
| HPV18REF.1_1_71 | ATAAGTCAGTTACCGAAGCCCGTTT | | |
| | GCGTCGTGCGGCACGGTGGGATACC | | |
| | ATACTTTTA | | |
| | TTACAAAAATACAAAAAGCATAAAA | | |
| | GGTATAAAACA | | |
| 625339_25353837_60975_ | CGCACGACGCAAACGGGCTTCGGTA | 120 | 511 |
| HPV18REF.1_1_72 | ACTGACTTATATAAAACATGTAAAC | | |
| | AATCTGGTACATGTCCACCTGATGT | | |
| | TGTTCCTAA | | |
| | GGTGGAGGGCACCACGTTAGCAGAT | | |
| | AAAATATTGCA | | |
| 625339_25353837_60975_ | CCTGTACCACTGCCAGTACCTATGC | 120 | 512 |
| HPV18REF.1_1_73 | CAAGTCCACCCAAAAATATACCAAG | | |
| | GCTTGACCATTGCAATATTTTATCT | | |
| | GCTAACGTG | | |
| | GTGCCCTCCACCTTAGGAACAACAT | | |
| | CAGGTGGACAT | | |
| 625339_25353837_60975_ | ATGGTCAAGCCTTGGTATATTTTTG | 120 | 513 |
| HPV18REF.1_1_74 | GGTGGACTTGGCATAGGTACTGGCA | | |
| | GTGGTACAGGGGGTCGTACAGGGTA | | |
| | CATTCCAT | | |
| | TGGGTGGGCGTTCCAATACAGTGGT | | |
| | GGATGTTGGTCC | | |
| 625339_25353837_60975_ | AATGTAACAATAGATGGGTCTGTGG | 120 | 514 |
| HPV18REF.1_1_75 | GGCCCACAGGTTCAATAACCACTGG | | |
| | GGGACGTGTAGGACCAACATCCACC | | |
| | ACTGTATT | | |
| | GGAACGCCCACCCAATGGAATGTAC | | |
| | CCTGTACGACCC | | |
| 625339_25353837_60975_ | TACACGTCCCCCAGTGGTTATTGAA | 120 | 515 |
| HPV18REF.1_1_76 | CCTGTGGGCCCCACAGACCCATCTA | | |
| | TTGTTACATTAATAGAGGACTCCAG | | |
| | TGTGGTTAC | | |
| | ATCAGGTGCACCTAGGCCTACGTTT | | |
| | ACTGGCACGTC | | |
| 625339_25353837_60975_ | GAAGGTGTGATATCCAAAACCGCAG | 120 | 516 |
| HPV18REF.1_1_77 | GTGTAGTTGTACCCGCAGATGTTAT | | |
| | ATCAAACCCAGACGTGCCAGTAAAC | | |
| | GTAGGCCT | | |
| | AGGTGCACCTGATGTAACCACACTG | | |
| | GAGTCCTCTATT | | |
| 625339_25353837_60975_ | TGGGTTTGATATAACATCTGCGGGT | 120 | 517 |
| HPV18REF.1_1_78 | ACAACTACACCTGCGGTTTTGGATA | | |
| | TCACACCTTCGTCTACCTCTGTGTC | | |
| | TATTTCCAC | | |
| | AACCAATTTTACCAATCCTGCATTT | | |
| | TCTGATCCGTC | | |
| 625339_25353837_60975_ | GTAGGGGTACCAACAAATACATTAC | 120 | 518 |
| HPV18REF.1_1_79 | CTGCCACCTCCCCAGTTTGTGGAAC | | |
| | TTCAATAATGGACGGATCAGAAAAT | | |
| | GCAGGATT | | |
| | GGTAAAATTGGTTGTGGAAATAGAC | | |
| | ACAGAGGTAGAC | | |
| 625339_25353837_60975_ | CATTATTGAAGTTCCACAAACTGGG | 120 | 519 |
| HPV18REF.1_1_80 | GAGGTGGCAGGTAATGTATTTGTTG | | |
| | GTACCCCTACATCTGGAACACATGG | | |
| | GTATGAGG | | |
| | AAATACCTTTACAAACATTTGCTTC | | |
| | TTCTGGTACGGG | | |
| 625339_25353837_60975_ | AGGCGGGGACCTGCTACACGCCGCA | 120 | 520 |
| HPV18REF.1_1_81 | CAGTAGGCAATGGGGTACTACTAAT | | |
| | GGGTTCCTCCCCCGTACCAGAAGAA | | |
| | GCAAATG | | |
| | TTTGTAAAGGTATTTCCTCATACCC | | |
| | ATGTGTTCCAGAT | | |
| 625339_25353837_60975_ | GGAGGAACCCATTAGTAGTACCCCA | 120 | 521 |
| HPV18REF.1_1_82 | TTGCCTACTGTGCGGCGTGTAGCAG | | |
| | GTCCCCGCCTTTACAGTAGGGCCTA | | |
| | CCAACAA | | |
| | GTGTCAGTGGCTAACCCTGAGTTTC | | |
| | TTACACGTCCATC | | |
| 625339_25353837_60975_ | TCAAATGTTAATGTAGTGTCCACAG | 120 | 522 |
| HPV18REF.1_1_83 | GCTCAAAGGCCGGGTTGTCATATGT | | |
| | AATTAAAGAGGATGGACGTGTAAGA | | |
| | AACTCAGG | | |
| | GTTAGCCACTGACACTTGTTGGTAG | | |
| | GCCCTACTGTAA | | |
| 625339_25353837_60975_ | CTCTTTAATTACATATGACAACCCG | 120 | 523 |
| HPV18REF.1_1_84 | GCCTTTGAGCCTGTGGACACTACAT | | |
| | TAACATTTGATCCTCGTAGTGATGT | | |
| | TCCTGATTCA | | |
| | GATTTTATGGATATTATCCGTCTAC | | |
| | ATAGGCCTGC | | |
| 625339_25353837_60975_ | AACATAGTTGCCCGTTGACCTAATC | 120 | 524 |
| HPV18REF.1_1_85 | TACTAAAGCGAACAGTCCCACGCCT | | |
| | GGATGTTAAAGCAGGCCTATGTAGA | | |
| | CGGATAATA | | |
| | TCCATAAAATCTGAATCAGGAACAT | | |
| | CACTACGAGGA | | |
| 625339_25353837_60975_ | TTTAACATCCAGGCGTGGGACTGTT | 120 | 525 |
| HPV18REF.1_1_86 | CGCTTTAGTAGATTAGGTCAACGGG | | |
| | CAACTATGTTTACCCGCAGCGGTAC | | |
| | ACAAATAG | | |
| | GTGCTAGGGTTCACTTTTATCATGA | | |
| | TATAAGTCCTAT | | |
| 625339_25353837_60975_ | TCATTGTCCTCCGTGGCAGATACTA | 120 | 526 |
| HPV18REF.1_1_87 | AAGGCTGCAGTTCAATATATTCTGG | | |
| | GGAAGGTGCAATAGGACTTATATCA | | |
| | TGATAAAAG | | |
| | TGAACCCTAGCACCTATTTGTGTAC | | |
| | CGCTGCGGGTA | | |
| 625339_25353837_60975_ | TGCACCTTCCCCAGAATATATTGAA | 120 | 527 |
| HPV18REF.1_1_88 | CTGCAGCCTTTAGTATCTGCCACGG | | |
| | AGGACAATGACTTGTTTGATATATA | | |
| | TGCAGATGA | | |
| | CATGGACCCTGCAGTGCCTGTACCA | | |
| | TCGCGTTCTAC | | |
| 625339_25353837_60975_ | TTACTATAGGAAGAGGCAGAAGATA | 120 | 528 |
| HPV18REF.1_1_89 | TAGTGGGCGAATATTTAAAAAATGC | | |
| | AAAGGAGGTAGTAGAACGCGATGGT | | |
| | ACAGGCAC | | |
| | TGCAGGGTCCATGTCATCTGCATAT | | |
| | ATATCAAACAAG | | |
| 625339_25353837_60975_ | TACCTCCTTTGCATTlliTAAATAT | 120 | 529 |
| HPV18REF.1_1_90 | TCGCCCACTATATCTTCTGCCTCTT | | |
| | CCTATAGTAATGTAACGGTCCCTTT | | |
| | AACCTCCTCTT | | |
| | GGGATGTGCCTGTATACACGGGTCC | | |
| | TGATATTAC | | |
| 625339_25353837_60975_ | TGTGTAGAGGCAGGGGCCGTGGGTG | 120 | 530 |
| HPV18REF.1_1_91 | ATACAATGGGCCATACAGAGGTAGT | | |
| | AGATGGTAATGTAATATCAGGACCC | | |
| | GTGTATA | | |
| | CAGGCACATCCCAAGAGGAGGTTAA | | |
| | AGGGACCGTTACA | | |
| 625339_25353837_60975_ | ATTACCATCTACTACCTCTGTATGG | 120 | 531 |
| HPV18REF.1_1_92 | CCCATTGTATCACCCACGGCCCCTG | | |
| | CCTCTACACAGTATATTGGTATACA | | |
| | TGGTACACA | | |
| | TTATTATTTGTGGCCATTATATTAT | | |
| | TTTATTCCTAA | | |
| 625339_25353837_60975_ | TATTGTCACTAGGCCGCCACAAAGC | 120 | 532 |
| HPV18REF.1_1_93 | CATCTGCAAAAAAATAGGGAACACG | | |
| | TTTACGTTTCTTAGGAATAAAATAA | | |
| | TATAATGGC | | |
| | CACAAATAATAATGTGTACCATGTA | | |
| | TACCAATATAC | | |
| 625339_25353837_60975_ | GAAACGTAAACGTGTTCCCTATTTT | 120 | 533 |
| HPV18REF.1_1_94 | TTTGCAGATGGCTTTGTGGCGGCCT | | |
| | AGTGACAATACCGTATATCTTCCAC | | |
| | CTCCTTCTG | | |
| | TGGCAAGAGTTGTAAATACCGATGA | | |
| | TTATGTGACTC | | |
| 625339_25353837_60975_ | AATATGGATTACCAACAGTTAATAA | 120 | 534 |
| HPV18REF.1_1_95 | TCTAGAGCTGCCAGCATGATAAAAT | | |
| | ATGCTTGTGCGAGTCACATAATCAT | | |
| | CGGTATTTAC | | |
| | AACTCTTGCCACAGAAGGAGGTGGA | | |
| | AGATATACGG | | |
| 625339_25353837_60975_ | GCACAAGCATATTTTATCATGCTGG | 120 | 535 |
| HPV18REF.1_1_96 | CAGCTCTAGATTATTAACTGTTGGT | | |
| | AATCCATATTTTAGGGTTCCTGCAG | | |
| | GTGGTGGCA | | |
| | ATAAGCAGGATATTCCTAAGGTTTC | | |
| | TGCATACCAAT | | |
| 625339_25353837_60975_ | TACTAGTATCAGGTAAACCAAATTT | 120 | 536 |
| HPV18REF.1_1_97 | ATTTGGGTCAGGTAACTGCACCCTA | | |
| | AATACTCTATATTGGTATGCAGAAA | | |
| | CCTTAGGAAT | | |
| | ATCCTGCTTATTGCCACCACCTGCA | | |
| | GGAACCCTAA | | |
| 625339_25353837_60975_ | ATAGAGTATTTAGGGTGCAGTTACC | 120 | 537 |
| HPV18REF.1_1_98 | TGACCCAAATAAATTTGGTTTACCT | | |
| | GATACTAGTATTTATAATCCTGAAA | | |
| | CACAACGTTT | | |
| | AGTGTGGGCCTGTGCTGGAGTGGAA | | |
| | ATTGGCCGTG | | |
| 625339_25353837_60975_ | CAGTGTCATCTAATTTATTATAAAA | 120 | 538 |
| HPV18REF.1_1_99 | TGGATGCCCACTAAGGCCAACACCT | | |
| | AAAGGCTGACCACGGCCAATTTCCA | | |
| | CTCCAGCAC | | |
| | AGGCCCACACTAAACGTTGTGTTTC | | |
| | AGGATTATAAA | | |
| 625339_25353837_60975_ | GTCAGCCTTTAGGTGTTGGCCTTAG | 120 | 539 |
| HPV18REF.1_1_100 | TGGGCATCCATTTTATAATAAATTA | | |
| | GATGACACTGAAAGTTCCCATGCCG | | |
| | CCACGTCTA | | |
| | ATGTTTCTGAGGACGTTAGGGACAA | | |
| | TGTGTCTGTAG | | |
| 625339_25353837_60975_ | CCCAGTGTTCCCCAATAGCAGGGGC | 120 | 540 |
| HPV18REF.1_1_101 | ACAGCCCAAAATACATAACTGTGTC | | |
| | TGCTTATAATCTACAGACACATTGT | | |
| | CCCTAACGT | | |
| | CCTCAGAAACATTAGACGTGGCGGC | | |
| | ATGGGAACTTT | | |
| 625339_25353837_60975_ | ATTATAAGCAGACACAGTTATGTAT | 120 | 541 |
| HPV18REF.1_1_102 | TTTGGGCTGTGCCCCTGCTATTGGG | | |
| | GAACACTGGGCTAAAGGCACTGCTT | | |
| | GTAAATCG | | |
| | CGTCCTTTATCACAGGGCGATTGCC | | |
| | CCCCTTTAGAAC | | |
| 625339_25353837_60975_ | AGTCCATGGCACCATATCCAGTATC | 120 | 542 |
| HPV18REF.1_1_103 | TACCATATCACCATCTTCCAAAACT | | |
| | GTGTTTTTAAGTTCTAAAGGGGGGC | | |
| | AATCGCCCT | | |
| | GTGATAAAGGACGCGATTTACAAGC | | |
| | AGTGCCTTTAG | | |
| 625339_25353837_60975_ | TTAAAAACACAGTTTTGGAAGATGG | 120 | 543 |
| HPV18REF.1_1_104 | TGATATGGTAGATACTGGATATGGT | | |
| | GCCATGGACTTTAGTACATTGCAAG | | |
| | ATACTAAAT | | |
| | GTGAGGTACCATTGGATATTTGTCA | | |
| | GTCTATTTGTA | | |
| 625339_25353837_60975_ | AGCAAAAAAACATGGAATCCCCATA | 120 | 544 |
| HPV18REF.1_1_105 | AGGATCTGCAGACATTTGTAAATAA | | |
| | TCAGGATATTTACAAATAGACTGAC | | |
| | AAATATCCAA | | |
| | TGGTACCTCACATTTAGTATCTTGC | | |
| | AATGTACTAA | | |
| 625339_25353837_60975_ | AATATCCTGATTATlTACAAATGTC | 120 | 545 |
| HPV18REF.1_1_106 | TGCAGATCCTTATGGGGATTCCATG | | |
| | TTTTTTTGCTTACGGCGTGAGCAGC | | |
| | TTTTTGCTAG | | |
| | GCATTTTTGGAATAGAGCAGGTACT | | |
| | ATGGGTGACA | | |
| 625339_25353837_60975_ | CACAGCTGCCAGGTGAAGCACGCAT | 120 | 546 |
| HPV18REF.1_1_107 | ACCTGTGCCTTTAATATATAAGGAT | | |
| | TGAGGCACAGTGTCACCCATAGTAC | | |
| | CTGCTCTA | | |
| | TTCCAAAAATGCCTAGCAAAAAGCT | | |
| | GCTCACGCCGTA | | |
| 625339_25353837_60975_ | CTGTGCCTCAATCCTTATATATTAA | 120 | 547 |
| HPV18REF.1_1_108 | AGGCACAGGTATGCGTGCTTCACCT | | |
| | GGCAGCTGTGTGTATTCTCCCTCTC | | |
| | CAAGTGGCT | | |
| | CTATTGTTACCTCTGACTCCCAGTT | | |
| | GTTTAATAAAC | | |
| 625339_25353837_60975_ | ATAATTGATTATGCCAGCAAACACC | 120 | 548 |
| HPV18REF.1_1_109 | ATTGTTATGACCCTGTGCCTTATGT | | |
| | AACCAATATGGTTTATTAAACAACT | | |
| | GGGAGTCAGA | | |
| | GGTAACAATAGAGCCACTTGGAGAG | | |
| | GGAGAATACA | | |
| 625339_25353837_60975_ | CATATTGGTTACATAAGGCACAGGG | 120 | 549 |
| HPV18REF.1_1_110 | TCATAACAATGGTGTTTGCTGGCAT | | |
| | AATCAATTATTTGTTACTGTGGTAG | | |
| | ATACCACTC | | |
| | GCAGTACCAATTTAACAATATGTGC | | |
| | TTCTACACAGT | | |
| 625339_25353837_60975_ | CCTCAACATGTCTGCTATACTGCTT | 120 | 550 |
| HPV18REF.1_1_111 | AAATTTGGTAGCATCATATTGCCCA | | |
| | GGTACAGGAGACTGTGTAGAAGCAC | | |
| | ATATTGTTA | | |
| | AATTGGTACTGCGAGTGGTATCTAC | | |
| | CACAGTAACAA | | |
| 625339_25353837_60975_ | CTCCTGTACCTGGGCAATATGATGC | 120 | 551 |
| HPV18REF.1_1_112 | TACCAAATTTAAGCAGTATAGCAGA | | |
| | CATGTTGAGGAATATGATTTGCAGT | | |
| | TTATTTTTCA | | |
| | GTTGTGTACTATTACTTTAACTGCA | | |
| | GATGTTATGT | | |
| 625339_25353837_60975_ | GGGGGGGAACACCAAAGTTCCAATC | 120 | 552 |
| HPV18REF.1_1_113 | CTCTAAAATACTGCTATTCATACTA | | |
| | TGAATATAGGACATAACATCTGCAG | | |
| | TTAAAGTAAT | | |
| | AGTACACAACTGAAAAATAAACTGC | | |
| | AAATCATATT | | |
| 625339_25353837_60975_ | CCTATATTCATAGTATGAATAGCAG | 120 | 553 |
| HPV18REF.1_1_114 | TATTTTAGAGGATTGGAACTTTGGT | | |
| | GTTCCCCCCCCGCCAACTACTAGTT | | |
| | TGGTGGATA | | |
| | CATATCGTTTTGTACAATCTGTTGC | | |
| | TATTACCTGTC | | |
| 625339_25353837_60975_ | CATTCCAAAACTTTAACTTATCATA | 120 | 554 |
| HPV18REF.1_1_115 | GGGATCCTTATTTTCAGCCGGTGCA | | |
| | GCATCCTTTTGACAGGTAATAGCAA | | |
| | CAGATTGTAC | | |
| | AAAACGATATGTATCCACCAAACTA | | |
| | GTAGTTGGCG | | |
| 625339_25353837_60975_ | AAAAGGATGCTGCACCGGCTGAAAA | 120 | 555 |
| HPV18REF.1_1_116 | TAAGGATCCCTATGATAAGTTAAAG | | |
| | TTTTGGAATGTGGATTTAAAGGAAA | | |
| | AGTTTTCTT | | |
| | TAGACTTAGATCAATATCCCCTTGG | | |
| | ACGTAAATTTT | | |
| 625339_25353837_60975_ | ATGGAGCAGAACGTTTGCGAGGGCC | 120 | 556 |
| HPV18REF.1_1_117 | TATGGTGGGCTTGCGACGCAATCCA | | |
| | GCCTGAACCAAAAATTTACGTCCAA | | |
| | GGGGATA | | |
| | TTGATCTAAGTCTAAAGAAAACTTT | | |
| | TCCTTTAAATCCA | | |
| 625339_25353837_60975_ | TGGTTCAGGCTGGATTGCGTCGCAA | 120 | 557 |
| HPV18REF.1_1_118 | GCCCACCATAGGCCCTCGCAAACGT | | |
| | TCTGCTCCATCTGCCACTACGTCTT | | |
| | CTAAACCT | | |
| | GCCAAGCGTGTGCGTGTACGTGCCA | | |
| | GGAAGTAATATG | | |
| 625339_25353837_60975_ | ACAAACACAAACACAGGACATACAA | 120 | 558 |
| HPV18REF.1_1_119 | ACACAACAATAGATGTATATATATA | | |
| | TACACACACACATATTACTTCCTGG | | |
| | CACGTACACG | | |
| | CACACGCTTGGCAGGTTTAGAAGAC | | |
| | GTAGTGGCAG | | |
| 625339_25353837_60975_ | TGTGTGTGTATATATATATACATCT | 120 | 559 |
| HPV18REF.1_1_120 | ATTGTTGTGTTTGTATGTCCTGTGT | | |
| | TTGTGTTTGTTGTATGATTGCATTG | | |
| | TATGGTATGT | | |
| | ATGGTTGTTGTTGTATGTTGTATGT | | |
| | TACTATATTT | | |
| 625339_25353837_60975_ | GGCGCAACCACATAACACACAGAAC | 120 | 560 |
| HPV18REF.1_1_121 | CACAAAACATATTTTATTTAATGCC | | |
| | ACATACCAACAAATATAGTAACATA | | |
| | CAACATACAA | | |
| | CAACAACCATACATACCATACAATG | | |
| | CAATCATACA | | |
| 625339_25353837_60975_ | GTTGGTATGTGGCATTAAATAAAAT | 120 | 561 |
| HPV18REF.1_1_122 | ATGTTTTGTGGTTCTGTGTGTTATG | | |
| | TGGTTGCGCCCTAGTGAGTAACAAC | | |
| | TGTATTTGT | | |
| | GTTTGTGGTATGGGTGTTGCTTGTT | | |
| | GGGCTATATAT | | |
| 625339_25353837_60975_ | GATTGTAGGATAAAATGGATGCTGT | 120 | 562 |
| HPV18REF.1_1_123 | AAGGTGTGCAGTTTTATAACTTGAA | | |
| | ATACAGGACAATATATAGCCCAACA | | |
| | AGCAACACC | | |
| | CATACCACAAACACAAATACAGTTG | | |
| | TTACTCACTAG | | |
| 625339_25353837_60975_ | TGTCCTGTATTTCAAGTTATAAAAC | 120 | 563 |
| HPV18REF.1_1_124 | TGCACACCTTACAGCATCCATTTTA | | |
| | TCCTACAATCCTCCATTTTGCTGTG | | |
| | CAACCGATTT | | |
| | CGGTTGCCTTTGGCTTATGTCTGTG | | |
| | GTTTTCTGCA | | |
| 625339_25353837_60975_ | AATATGTAGGAGCAGTGCCCAAAAG | 120 | 564 |
| HPV18REF.1_1_125 | ATTAAAGTTTGCAATAGTGCCAGCG | | |
| | TACTGTATTGTGCAGAAAACCACAG | | |
| | ACATAAGCC | | |
| | AAAGGCAACCGAAATCGGTTGCACA | | |
| | GCAAAATGGAG | | |
| 625339_25353837_60975_ | CAATACAGTACGCTGGCACTATTGC | 120 | 565 |
| HPV18REF.1_1_126 | AAACTTTAATCTTTTGGGCACTGCT | | |
| | CCTACATATTTTGAACAATTGGCGC | | |
| | GCCTCTTTG | | |
| | GCGCATATAAGGCGCACCTGGTATT | | |
| | AGTCATTTTCC | | |
| 625339_25353837_60975_ | GTTTTATTACTTAGGGAGTGGATAT | 120 | 566 |
| HPV18REF.1_1_127 | AGTTATGCAAGCAATTGTTGTAGCG | | |
| | CACCTGGACAGGAAAATGACTAATA | | |
| | CCAGGTGCG | | |
| | CCTTATATGCGCCAAAGAGGCGCGC | | |
| | CAATTGTTCAA | | |
| 625339_25353837_60975_ | TGTCCAGGTGCGCTACAACAATTGC | 120 | 567 |
| HPV18REF.1_1_128 | TTGCATAACTATATCCACTCCCTAA | | |
| | GTAATAAAACTGCTTTTAGGCACAT | | |
| | ATTTTAGTTT | | |
| | GTTTTTACTTAAGCTAATTGCATAC | | |
| | TTGGCTTGTA | | |
| 625339_25353837_60975_ | GCTTAGTCATATTATAGTTCATGTT | 120 | 568 |
| HPV18REF.1_1_129 | AAGGGTAGACAGAATGTTGGACATG | | |
| | AAAGTAGTTGTACAAGCCAAGTATG | | |
| | CAATTAGCT | | |
| | TAAGTAAAAACAAACTAAAATATGT | | |
| | GCCTAAAAGCA | | |
| 625339_25353837_60975_ | CAACTACTTTCATGTCCAACATTCT | 117 | 569 |
| HPV18REF.1_1_130 | GTCTACCCTTAACATGAACTATAAT | | |
| | ATGACTAAGCTGTGCATACATAGTT | | |
| | TATGCAACCG | | |
| | AAATAGGTTGGGCAGCACATACTAT | | |
| | ACTTTTC | | |

| HPV31_DoubleStrand_ | | | SEQ |
|---|---|---|---|
| SequenceName | Sequence | Length | ID#: |
| 625339_25353838_333048_ | GATTTTTGAACATGGCGTCTGTAGG | 120 | 57 |
| HPV31REF.1_1_1 | TTTGCACAAAATACTATGTGCTTTA | | |
| | TATACCAACCGTTTTCGGTTCACCA | | |
| | CTTTCGGTCACTCCCTACTTTTTT | | |
| | ATACTAAGATTATTATTATTA | | |
| 625339_25353838_333048_ | GGTTGGTATATAAAGCACATAGTAT | 120 | 57 |
| HPV31REF.1_1_2 | TTTGTGCAAACCTACAGACGCCATG | | |
| | TTCAAAAATCCTGCAGAAAGACCTC | | |
| | GGAAATTGCATGAACTAAGCTC | | |
| | GGCATTGGAAATACCCTACGATG | | |
| 625339_25353838_333048_ | AATCTAATACCTCTGTTTCTGTTAA | 120 | 57 |
| HPV31REF.1_1_3 | CTGACCTTTGCAGTAGACACAATTC | | |
| | AATCTTAGTTCATCGTAGGGTATTT | | |
| | CCAATGCCGAGCTTAGTTCATGC | | |
| | AATTTCCGAGGTCTTTCTGCAG | | |
| 625339_25353838_333048_ | AACTAAGATTGAATTGTGTCTACTG | 120 | 57 |
| HPV31REF.1_1_4 | CAAAGGTCAGTTAACAGAAACAGAG | | |
| | GTATTAGATTTTGCATTTACAGATT | | |
| | TAACAATAGTATATAGGGACGAC | | |
| | ACACCACACGGAGTGTGTACAA | | |
| 625339_25353838_333048_ | CATACACACTATATCTATACCATCT | 120 | 57 |
| HPV31REF.1_1_5 | AAATTCACTTACTTTTGAATAAAAT | | |
| | CTTAAACATTTTGTACACACTCCGT | | |
| | GTGGTGTGTCGTCCCTATATACTAT | | |
| | TGTTAAATCTGTAAATGCAA | | |
| 625339_25353838_333048_ | AATGTTTAAGATTTTATTCAAAAGT | 120 | 57 |
| HPV31REF.1_1_6 | AAGTGAATTTAGATGGTATAGATAT | | |
| | AGTGTGTATGGAACAACATTAGAAA | | |
| | AATTGACAAACAAAGGTATATGTG | | |
| | ATTTGTTAATTAGGTGTATAA | | |
| 625339_25353838_333048_ | ATCGTTTCTTTTTATCCAAATGTCT | 120 | 57 |
| HPV31REF.1_1_7 | TTGTTTTTCTTCTGGACACAACGGT | | |
| | CTTTGACACGTTATACACCTAATTA | | |
| | ACAAATCACATATACCTTTGTTTGT | | |
| | CAATTTTTCTAATGTTGTTC | | |
| 625339_25353838_333048_ | CGTGTCAAAGACCGTTGTGTCCAGA | 120 | 57 |
| HPV31REF.1_1_8 | AGAAAAACAAAGACATTTGGATAAA | | |
| | AAGAAACGATTCCACAACATAGGAG | | |
| | GAAGGTGGACAGGACGTTGCA | | |
| | TAGCATGTTGGAGAAGACCTCGTA | | |
| 625339_25353838_333048_ | TCTAACACATAGTCTTGCAACGTAG | 120 | 57 |
| HPV31REF.1_1_9 | GTGTTTCTCCACGCATGTTTACACT | | |
| | TGGGTTTCAGTACGAGGTCTTCTCC | | |
| | AACATGCTATGCAACGTCCTGT | | |
| | CCACCTTCCTCCTATGTTGTGGA | | |
| 625339_25353838_333048_ | CTGAAACCCAAGTGTAAACATGCGT | 120 | 57 |
| HPV31REF.1_1_10 | GGAGAAACACCTACGTTGCAAGACT | | |
| | ATGTGTTAGATTTGCAACCTGAGGC | | |
| | AACTGACCTCCACTGTTATGA | | |
| | GCAATTACCCGACAGCTCAGATGA | | |
| 625339_25353838_333048_ | ATATTGTAATTGGATGTGTCCGGTT | 120 | 58 |
| HPV31REF.1_1_11 | CTGCTTGTCCAGCTGGACTGTCTAT | | |
| | GACATCCTCCTCATCTGAGCTGTCG | | |
| | GGTAATTGCTCATAACAGTGGA | | |
| | GGTCAGTTGCCTCAGGTTGCAAA | | |
| 625339_25353838_333048_ | GGAGGATGTCATAGACAGTCCAGCT | 120 | 58 |
| HPV31REF.1_1_12 | GGACAAGCAGAACCGGACACATCCA | | |
| | ATTACAATATCGTTACCTTTTGTTG | | |
| | TCAGTGTAAGTCTACACTTCGT | | |
| | TTGTGTGTACAGAGCACACAAGT | | |
| 625339_25353838_333048_ | CAGTTGGGGCACACGATTCCAAATG | 120 | 58 |
| HPV31REF.1_1_13 | AGCCCATTAACAGCTCTTGCAATAT | | |
| | GCGAATATCTACTTGTGTGCTCTGT | | |
| | ACACACAAACGAAGTGTAGACT | | |
| | TACACTGACAACAAAAGGTAACG | | |
| 625339_25353838_333048_ | AGATATTCGCATATTGCAAGAGCTG | 120 | 58 |
| HPV31REF.1_1_14 | TTAATGGGCTCATTTGGAATCGTGT | | |
| | GCCCCAACTGTTCTACTAGACTGTA | | |
| | ACTACAATGGCTGATCCAGCAG | | |
| | GTACAGATGGGGAGGGGACGGGA | | |
| 625339_25353838_333048_ | TGAAATGTTGTCCCCTGTCTGTCTG | 120 | 58 |
| HPV31REF.1_1_15 | TCAATTACTGCTTCTACATAAAACC | | |
| | AACCATTGCATCCCGTCCCCTCCCC | | |
| | ATCTGTACCTGCTGGATCAGCC | | |
| | ATTGTAGTTACAGTCTAGTAGAA | | |
| 625339_25353838_333048_ | TGCAATGGTTGGTTTTATGTAGAAG | 120 | 58 |
| HPV31REF.1_1_16 | CAGTAATTGACAGACAGACAGGGGA | | |
| | CAACATTTCAGAGGACGAAAATGAA | | |
| | GACAGTAGTGATACTGGGGAG | | |
| | GATATGGTTGACTTTATTGACAAT | | |
| 625339_25353838_333048_ | CTGTGCATGAAACAATGCCTGTGCT | 120 | 58 |
| HPV31REF.1_1_17 | GTCTCTGCTTCTGCCTGATTGTTGT | | |
| | ATACATTACAATTGTCAATAAAGTC | | |
| | AACCATATCCTCCCCAGTATCAC | | |
| | TACTGTCTTCATTTTCGTCCTC | | |
| 625339_25353838_333048_ | TGTAATGTATACAACAATCAGGCAG | 120 | 58 |
| HPV31REF.1_1_18 | AAGCAGAGACAGCACAGGCATTGTT | | |
| | TCATGCACAGGAAGCGGAGGAACAT | | |
| | GCAGAGGCTGTGCAGGTTCT | | |
| | AAAACGAAAGTATGTAGGTAGTCCT | | |
| 625339_25353838_333048_ | GCATATAGCTTTTAACCGTGGACTA | 120 | 58 |
| HPV31REF.1_1_19 | ATATTATAATCCACACAACTACTAA | | |
| | TATCACTTAAAGGACTACCTACATA | | |
| | CTTTCGTTTTAGAACCTGCACAGC | | |
| | CTCTGCATGTTCCTCCGCTTC | | |
| 625339_25353838_333048_ | TTAAGTGATATTAGTAGTTGTGTGG | 120 | 58 |
| HPV31REF.1_1_20 | ATTATAATATTAGTCCACGGTTAAA | | |
| | AGCTATATGCATAGAAAATAACAGT | | |
| | AAAACAGCAAAACGAAGACTCTTT | | |
| | GAACTTCCAGACAGCGGGTAT | | |
| 625339_25353838_333048_ | TAATGTTGTTTGTTGCTCCTCTACC | 120 | 59 |
| HPV31REF.1_1_21 | TGTACCATCTGCTGCGTTTCCACTT | | |
| | CAGTATTGCCATACCCGCTGTCTGG | | |
| | AAGTTCAAAGAGTCTTCGTTTTG | | |
| | CTGTTTTACTGTTATTTTCTAT | | |
| 625339_25353838_333048_ | GGCAATACTGAAGTGGAAACGCAGC | 120 | 59 |
| HPV31REF.1_1_22 | AGATGGTACAGGTAGAGGAGCAACA | | |
| | AACAACATTAAGTTGTAATGGTAGT | | |
| | GACGGGACACATAGTGAACG | | |
| | AGAGAATGAAACTCCAACACGTAAT | | |
| 625339_25353838_333048_ | TTCTTTAAATTTACCTAACATAGCA | 120 | 59 |
| HPV31REF.1_1_23 | GCTTTACCATTGCTAGTTTTTAACA | | |
| | CTTGCAATATATTACGTGTTGGAGT | | |
| | TTCATTCTCTCGTTCACTATGTGTC | | |
| | CCGTCACTACCATTACAACT | | |
| 625339_25353838_333048_ | ATATTGCAAGTGTTAAAAACTAGCA | 120 | 59 |
| HPV31REF.1_1_24 | ATGGTAAAGCTGCTATGTTAGGTAA | | |
| | ATTTAAAGAATTATATGGTGTAAGT | | |
| | TTTATGGAACTAATTAGGCCATTT | | |
| | CAAAGCAATAAAAGCACATGT | | |
| 625339_25353838_333048_ | GGTTTTAAATCCTTCTGCAACTGTA | 120 | 59 |
| HPV31REF.1_1_25 | CCTGTAACTCCAAACGCAGCTACAC | | |
| | ACCAATCAGTACATGTGCTTTTATT | | |
| | GCTTTGAAATGGCCTAATTAGTT | | |
| | CCATAAAACTTACACCATATAA | | |
| 625339_25353838_333048_ | ACTGATTGGTGTGTAGCTGCGTTTG | 120 | 59 |
| HPV31REF.1_1_26 | GAGTTACAGGTACAGTTGCAGAAGG | | |
| | ATTTAAAACCCTATTGCAACCATAT | | |
| | TGTTTGTATTGCCATTTACAAAG | | |
| | TTTAGCATGTTCCTGGGGCATG | | |
| 625339_25353838_333048_ | TAATTTTTCAATTGTTATTCTATTT | 120 | 59 |
| HPV31REF.1_1_27 | TTTGCACATTTAAATCTCACAAGCA | | |
| | TTAACATAACCATGCCCCAGGAACA | | |
| | TGCTAAACTTTGTAAATGGCAATAC | | |
| | AAACAATATGGTTGCAATAG | | |
| 625339_25353838_333048_ | GTTATGTTAATGCTTGTGAGATTTA | 120 | 59 |
| HPV31REF.1_1_28 | AATGTGCAAAAAATAGAATAACAAT | | |
| | TGAAAAATTATTAGAAAAATTATTG | | |
| | TGTATATCTACAAATTGTATGTTAA | | |
| | TTCAGCCACCCAAATTACGT | | |
| 625339_25353838_333048_ | ATATACATCGCTAATGTTTGACATT | 120 | 59 |
| HPV31REF.1_1_29 | CCTGTTCTGTACCAATATAATGCTG | | |
| | CAGCTGTGCTACGTAATTTGGGTGG | | |
| | CTGAATTAACATACAATTTGTAG | | |
| | ATATACACAATAATTTTTCTAA | | |
| 625339_25353838_333048_ | AGCACAGCTGCAGCATTATATTGGT | 120 | 59 |
| HPV31REF.1_1_30 | ACAGAACAGGAATGTCAAACATTAG | | |
| | CGATGTATATGGTGAAACACCAGAA | | |
| | TGGATAGAAAGACAAACAGTAT | | |
| | TACAGCATAGTTTTAATGACACA | | |
| 625339_25353838_333048_ | ACTATCATCCATAACATCATTGTCA | 120 | 60 |
| HPV31REF.1_1_31 | TATGCCCATTGTACCATTTGGGACA | | |
| | AATCAAATGTTGTGTCATTAAAACT | | |
| | ATGCTGTAATACTGTTTGTCTTTC | | |
| | TATCCATTCTGGTGTTTCACC | | |
| 625339_25353838_333048_ | ACATTTGATTTGTCCCAAATGGTAC | 120 | 60 |
| HPV31REF.1_1_32 | AATGGGCATATGACAATGATGTTAT | | |
| | GGATGATAGTGAAATTGCCTATAAA | | |
| | TATGCACAATTAGCTGACAGTGA | | |
| | TAGTAATGCATGTGCATTTTTA | | |
| 625339_25353838_333048_ | TTTATAATGTCTACACATTGTTCCA | 120 | 60 |
| HPV31REF.1_1_33 | CAATCTTTAACTATTTTTGCCTGCG | | |
| | AATTACTTTTTAAAAATGCACATGC | | |
| | ATTACTATCACTGTCAGCTAATTGT | | |
| | GCATATTTATAGGCAATTTC | | |
| 625339_25353838_333048_ | AAAAGTAATTCGCAGGCAAAAATAG | 120 | 60 |
| HPV31REF.1_1_34 | TTAAAGATTGTGGAACAATGTGTAG | | |
| | ACATTATAAACGAGCAGAAAAACGA | | |
| | CAAATGTCCATGGGACAGTGGA | | |
| | TTAAAAGTAGATGTGACAAAGTT | | |
| 625339_25353838_333048_ | AAATTCTATTTGTTGATATCTTAAA | 120 | 60 |
| HPV31REF.1_1_35 | AACTTTACTATGTCCCTCCAGTCAC | | |
| | CTTCGTCACTAACTTTGTCACATCT | | |
| | ACTTTTAATCCACTGTCCCATGGA | | |
| | CATTTGTCGTTTTTCTGCTCG | | |
| 625339_25353838_333048_ | AGTGACGAAGGTGACTGGAGGGACA | 120 | 60 |
| HPV31REF.1_1_36 | TAGTAAAGTTTTTAAGATATCAACA | | |
| | AATAGAATTTGTGTCATTTTTATCT | | |
| | GCATTAAAGCTGTTTTTAAAAGG | | |
| | AGTGCCAAAGAAAAACTGTATT | | |
| 625339_25353838_333048_ | AAAGCTAATAAGGCTCATTCCAAAA | 120 | 60 |
| HPV31REF.1_1_37 | TATGATTTACCTGTATTAGGTGCAC | | |
| | CATGTATTAAAATACAGTTTTTCTT | | |
| | TGGCACTCCTTTTAAAAACAGCTT | | |
| | TAATGCAGATAAAAATGACAC | | |
| 625339_25353838_333048_ | TTAATACATGGTGCACCTAATACAG | 120 | 60 |
| HPV31REF.1_1_38 | GTAAATCATATTTTGGAATGAGCCT | | |
| | TATTAGCTTTTTACAAGGATGTATA | | |
| | ATATCATATGCAAATTCAAAAAGT | | |
| | CATTTTTGGTTACAACCACTG | | |
| 625339_25353838_333048_ | GTCTATATAATGCCAACATGGCGTT | 120 | 60 |
| HPV31REF.1_1_39 | GTAGCATCATCTAACATGCCTATTT | | |
| | TAGCATCAGCCAGTGGTTGTAACCA | | |
| | AAAATGACTTTTTGAATTTGCAT | | |
| | ATGATATTATACATCCTTGTAA | | |
| 625339_25353838_333048_ | GCTGATGCTAAAATAGGCATGTTAG | 120 | 60 |
| HPV31REF.1_1_40 | ATGATGCTACAACGCCATGTTGGCA | | |
| | TTATATAGACAATTACCTACGAAAT | | |
| | GCACTAGATGGCAACCCTGTAT | | |
| | CTATAGATGTAAAGCATAAAGCT | | |
| 625339_25353838_333048_ | ATCCTTACCTGCATTTATATTAGAT | 120 | 61 |
| HPV31REF.1_1_41 | GTAATCAATAAAGGAGGACATTTTA | | |
| | ACTGCATTAAAGCTTTATGCTTTAC | | |
| | ATCTATAGATACAGGGTTGCCATC | | |
| | TAGTGCATTTCGTAGGTAATT | | |
| 625339_25353838_333048_ | TTAATGCAGTTAAAATGTCCTCCTT | 120 | 61 |
| HPV31REF.1_1_42 | TATTGATTACATCTAATATAAATGC | | |
| | AGGTAAGGATGACAGATGGCCATAC | | |
| | CTACATAGCAGACTGGTGGTTTT | | |
| | TACATTTCCAAATCCATTTCCA | | |
| 625339_25353838_333048_ | GAAAAAGGATTTCCAGTTTTTATCA | 120 | 61 |
| HPV31REF.1_1_43 | CTTAATTCATATACTGGATTTCCGT | | |
| | TTTTGTCAAATGGAAATGGATTTGG | | |
| | AAATGTAAAAACCACCAGTCTGCT | | |
| | ATGTAGGTATGGCCATCTGTC | | |
| 625339_25353838_333048_ | TTTGACAAAAACGGAAATCCAGTAT | 120 | 61 |
| HPV31REF.1_1_44 | ATGAATTAAGTGATAAAAACTGGAA | | |
| | ATCCTTTTTCTCAAGGACGTGGTGC | | |
| | AGATTAAATTTGCACGAGGAAGA | | |
| | GGACAAAGAAAACGATGGAGAC | | |
| 625339_25353838_333048_ | CTATCATTTTCATAATGTTCTAATA | 120 | 61 |
| HPV31REF.1_1_45 | TTTTGTCCTGACACACATTTAAACG | | |
| | TTGAGAAAGAGTCTCCATCGTTTTC | | |
| | TTTGTCCTCTTCCTCGTGCAAATTT | | |
| | AATCTGCACCACGTCCTTGA | | |
| 625339_25353838_333048_ | TCTTTCTCAACGTTTAAATGTGTGT | 120 | 61 |
| HPV31REF.1_1_46 | CAGGACAAAATATTAGAACATTATG | | |
| | AAAATGATAGTAAACGACTTTGTGA | | |
| | TCATATAGACTATTGGAAACATAT | | |
| | TCGACTTGAATGTGTATTAAT | | |
| 625339_25353838_333048_ | GACAACGCTGGCACCACCTGGTGGT | 120 | 61 |
| HPV31REF.1_1_47 | TAATACTGTGTATTCCCATTTCTCT | | |
| | TGCTTTATACATTAATACACATTCA | | |
| | AGTCGAATATGTTTCCAATAGTC | | |
| | TATATGATCACAAAGTCGTTTA | | |
| 625339_25353838_333048_ | GTATAAAGCAAGAGAAATGGGAATA | 120 | 61 |
| HPV31REF.1_1_48 | CACAGTATTAACX+ACCAGGTGGTG | | |
| | CCAGCGTTGTCAGTATCAAAGGCCA | | |
| | AAGCCTTACAAGCTATTGAACT | | |
| | ACAAATGATGTTGGAAACATTAAA | | |
| 625339_25353838_333048_ | AAATACAGTTCAAGACTTGTTTGCT | 120 | 61 |
| HPV31REF.1_1_49 | GCATTGTCCAGTCCTCATTTTTGTA | | |
| | TTCAGTGTTATTTAATGTTTCCAAC | | |
| | ATCATTTGTAGTTCAATAGCTTGTA | | |
| | AGGCTTTGGCCTTTGATACT | | |
| 625339_25353838_333048_ | TAACACTGAATACAAAAATGAGGAC | 120 | 61 |
| HPV31REF.1_1_50 | TGGACAATGCAGCAAACAAGTCTTG | | |
| | AACTGTATTTAACTGCACCTACAGG | | |
| | GTGTTTAAAAAAACATGGATATA | | |
| | CTGTAGAGGTGCAATTTGATGG | | |
| 625339_25353838_333048_ | CCATCTATACATAGGTATATAAATT | 120 | 62 |
| HPV31REF.1_1_51 | TCCAGTTAGTATAATGCATGGTGTT | | |
| | GTGTACATCACCATCAAATTGCACC | | |
| | TCTACAGTATATCCATGTTTTTTT | | |
| | AAACACCCTGTAGGTGCAGTT | | |
| 625339_25353838_333048_ | TGATGTACACAACACCATGCATTAT | 120 | 62 |
| HPV31REF.1_1_52 | ACTAACTGGAAATTTATATACCTAT | | |
| | GTATAGATGGCCAATGTACTGTTGT | | |
| | GGAAGGGCAAGTTAATTGTAAGG | | |
| | GCATTTATTATGTACATGAAGG | | |
| 625339_25353838_333048_ | TTTTTACCAGTCCCATATTTTTTTG | 120 | 62 |
| HPV31REF.1_1_53 | CCTCTTCTGTAAAATTTACAAAATA | | |
| | TGTTATATGTCCTTCATGTACATAA | | |
| | TAAATGCCCTTACAATTAACTTGCC | | |
| | CTTCCACAACAGTACATTGG | | |
| 625339_25353838_333048_ | ACATATAACATATTTTGTAAATTTT | 120 | 62 |
| HPV31REF.1_1_54 | ACAGAAGAGGCAAAAAAATATGGGA | | |
| | CTGGTAAAAAATGGGAAGTGCATGC | | |
| | GGGTGGTCAGGTAATTGTTTTTC | | |
| | CTGAATCTGTATTTAGCAGTGA | | |
| 625339_25353838_333048_ | GATGTGGTGGTGTTGTTGGCTGTTG | 120 | 62 |
| HPV31REF.1_1_55 | GTAGCTTTGTAACAATCCCAGCAAA | | |
| | GGATATTTCGTCACTGCTAAATACA | | |
| | GATTCAGGAAAAACAATTACCT | | |
| | GACCACCCGCATGCACTTCCCAT | | |
| 625339_25353838_333048_ | CGAAATATCCTTTGCTGGGATTGTT | 120 | 62 |
| HPV31REF.1_1_56 | ACAAAGCTACCAACAGCCAACAACA | | |
| | CCACCACATCGAATTCCAAAACCTG | | |
| | CGCCTTGGGCACCAGTGAAGG | | |
| | TGTGCGGCGGGCGACGACGTCTAC | | |
| 625339_25353838_333048_ | CGCAACAACTTGTTGGGGTGGTGGG | 120 | 62 |
| HPV31REF.1_1_57 | TGTTTCTGTGCTCTGGCTCTGTTCT | | |
| | TGGTCGCTTAGTAGACGTCGTCGCC | | |
| | CGCCGCACACCTTCACTGGT | | |
| | GCCCAAGGCGCAGGTTTTGGAATTC | | |
| 625339_25353838_333048_ | TAAGCGACCAAGAACAGAGCCAGAG | 120 | 62 |
| HPV31REF.1_1_58 | CACAGAAACACCCACCACCCCAACA | | |
| | AGTTGTTGCGAGGCGACTCCGTGGA | | |
| | CAGTGTCAACTGTGGGGTTA | | |
| | TCAGTGCAGCTGCATGCACAAACCA | | |
| 625339_25353838_333048_ | TTTGCATCACCTTTTAAGTGTATTA | 120 | 62 |
| HPV31REF.1_1_59 | TAGGTGTAGTTGCAGGACAACTGAC | | |
| | AGCCCTTGTTTGGTTTGTGCATGCA | | |
| | GCTGCACTGATAACCCCACAGT | | |
| | TGACACTGTCCACGGAGTCGCCT | | |
| 625339_25353838_333048_ | AACAAGGGCTGTCAGTTGTCCTGCA | 120 | 62 |
| HPV31REF.1_1_60 | ACTACACCTATAATACACTTAAAAG | | |
| | GTGATGCAAATATATTAAAATGTTT | | |
| | AAGATATAGGCTGTCAAAATATAA | | |
| | ACAATTGTATGAACAAGTGTC | | |
| 625339_25353838_333048_ | GTTAAGGTTACAATAGCATTTTTAT | 120 | 63 |
| HPV31REF.1_1_61 | GTTTTCCATCTGTACATGTCCAATG | | |
| | CCATGTAGATGACACTTGTTCATAC | | |
| | AATTGTTTATATTTTGACAGCCTA | | |
| | TATCTTAAACATTTTAATATA | | |
| 625339_25353838_333048_ | ATCTACATGGCATTGGACATGTACA | 120 | 63 |
| HPV31REF.1_1_62 | GATGGAAAACATAAAAATGCTATTG | | |
| | TAACCTTAACATATATAAGTACATC | | |
| | ACAAAGAGACGATTTTTTAAATAC | | |
| | TGTAAAAATACCTAACACAGT | | |
| 625339_25353838_333048_ | CTGTAGAAATATTTAGTTCAATCAT | 120 | 63 |
| HPV31REF.1_1_63 | TAGGCTAAATAGTCATATATCCTGT | | |
| | TGACACTGATACTGTGTTAGGTATT | | |
| | TTTACAGTATTTAAAAAATCGTCTC | | |
| | TTTGTGATGTACTTATATAT | | |
| 625339_25353838_333048_ | ATCAGTGTCAACAGGATATATGACT | 120 | 63 |
| HPV31REF.1_1_64 | ATTTAGCCTAATGATTGAACTAAAT | | |
| | ATTTCTACAGTAAGCATTGTGCTAT | | |
| | GCTTTTTGCTTTGCTTTTGTGTGC | | |
| | TACTATTTGTGTGTCTTGTCA | | |
| 625339_25353838_333048_ | AAATCACAATTAATAATAGTAGTGT | 120 | 63 |
| HPV31REF.1_1_65 | TGCATATACCGACACAGACAGCACA | | |
| | AGTGGACGTATGACAAGACACACAA | | |
| | ATAGTAGCACACAAAAGCAAAG | | |
| | CAAAAAGCATAGCACAATGCTTA | | |
| 625339_25353838_333048_ | TACGTCCACTTGTGCTGTCTGTGTC | 120 | 63 |
| HPV31REF.1_1_66 | GGTATATGCAACACTACTATTATTA | | |
| | ATTGTGATTTTATGGGTTATTGCAA | | |
| | CCTCTCCATTACGTTGTTTTTGTA | | |
| | TATATGTTGTGTTTATATATA | | |
| 625339_25353838_333048_ | AGTAAAAAAGTTACTGTTGACTTAA | 120 | 63 |
| HPV31REF.1_1_67 | AAAAGATGCATGTGTATGAATTACA | | |
| | AATAATGGAATATATATAAACACAA | | |
| | CATATATACAAAAACAACGTAATG | | |
| | GAGAGGTTGCAATAACCCATA | | |
| 625339_25353838_333048_ | TTCCATTATTTGTAATTCATACACA | 120 | 63 |
| HPV31REF.1_1_68 | TGCATCTTTTTTAAGTCAACAGTAA | | |
| | CTTTTTTACTTGTGTATACTGTTGT | | |
| | TTgtattggtattggtattggtatt | | |
| | ggtattggtattggt | | |
| | atAAT | | |
| 625339_25353838_333048_ | AGTGCGTTTTGTAGAGCGTTTGGAC | 120 | 63 |
| HPV31REF.1_1_69 | CGCATGGTAATAATAAAAAAAAAGT | | |
| | AAAAAAGTTTATTATACCAATACCA | | |
| | ATACCAATACCAATACCAATACC | | |
| | AATACAAACAACAGTATACACA | | |
| 625339_25353838_333048_ | AAACTTTTTTACTTT | 120 | 63 |
| HPV31REF.1_1_70 | TTTTTTATTATTACCATGCG | | |
| | GTCCAAACGCTCTACAAAACGCACT | | |
| | AAACGTGCGTCTGCTACACAATTAT | | |
| | ATCAAACATGTAAA | | |
| | GCAGCAGGTACTTGTCCATCA | | |
| 625339_25353838_333048_ | ACTACCATACCTTAATATTTGGTCT | 120 | 64 |
| HPV31REF.1_1_71 | GCAATGGTAGTATGTTCTATTTTAG | | |
| | GTATAACGTCTGATGGACAAGTACC | | |
| | TGCTGCTTTACATGTTTGATATAA | | |
| | TTGTGTAGCAGACGCACGTTT | | |
| 625339_25353838_333048_ | GACGTTATACCTAAAATAGAACATA | 120 | 64 |
| HPV31REF.1_1_72 | CTACCATTGCAGACCAAATATTAAG | | |
| | GTATGGTAGTATGGGTGTTTTTTTT | | |
| | GGTGGGTTGGGTATTGGGTCCG | | |
| | GCTCTGGTACTGGGGGTCGCACT | | |
| 625339_25353838_333048_ | TCTAATAGGTATACTTGCCTCAGAT | 120 | 64 |
| HPV31REF.1_1_73 | ACTGTAGAAGGACGTGTACTAAGAG | | |
| | GGACATATCCAGTGCGACCCCCAGT | | |
| | ACCAGAGCCGGACCCAATACC | | |
| | CAACCCACCAAAAAAAACACCCAT | | |
| 625339_25353838_333048_ | GGATATGTCCCTCTTAGTACACGTC | 120 | 64 |
| HPV31REF.1_1_74 | CTTCTACAGTATCTGAGGCAAGTAT | | |
| | ACCTATTAGACCACCAGTTAGCATT | | |
| | GACCCTGTAGGTCCCTTGGACC | | |
| | CCTCTATAGTAAGTCTTGTTGAA | | |
| 625339_25353838_333048_ | AGATGTTGTAGGAGGGTGTGGTATA | 120 | 64 |
| HPV31REF.1_1_75 | GGAGCAGGGGCACCAACATCAACAA | | |
| | TTCCAGATTCTTCAACAAGACTTAC | | |
| | TATAGAGGGGTCCAAGGGAC | | |
| | CTACAGGGTCAATGCTAACTGGTGG | | |
| 625339_25353838_333048_ | GAATCTGGAATTGTTGATGTTGGTG | 120 | 64 |
| HPV31REF.1_1_76 | CCCCTGCTCCTATACCACACCCTCC | | |
| | TACAACATCTGGGTTTGACATTGCT | | |
| | ACAACTGCAGACACAACACCTG | | |
| | CAATTTTAGATGTAACAAGTGTT | | |
| 625339_25353838_333048_ | TGCAGGTGTAGGAGGCTGCAATACA | 120 | 64 |
| HPV31REF.1_1_77 | GATGGATCAGTAAAAGTAGGATTTT | | |
| | CATGTGTGCTAACACTTGTTACATC | | |
| | TAAAATTGCAGGTGTTGTGTCT | | |
| | GCAGTTGTAGCAATGTCAAACCC | | |
| 625339_25353838_333048_ | AGCACACATGAAAATCCTACTTTTA | 120 | 64 |
| HPV31REF.1_1_78 | CTGATCCATCTGTATTGCAGCCTCC | | |
| | TACACCTGCAGAAACATCAGGTCAT | | |
| | TTACTACTTTCATCATCATCTATT | | |
| | AGCACACATAATTATGAGGAA | | |
| 625339_25353838_333048_ | GGGTGTGCTACTTGTTATGTTTTCA | 120 | 64 |
| HPV31REF.1_1_79 | TTATTAGTAGAAACAATAAATGTAT | | |
| | CCATAGGTATTTCCTCATAATTATG | | |
| | TGTGCTAATAGATGATGATGAAAG | | |
| | TAGTAAATGACCTGATGTTTC | | |
| 625339_25353838_333048_ | ATACCTATGGATACATTTATTGTTT | 120 | 64 |
| HPV31REF.1_1_80 | CTACTAATAATGAAAACATAACAAG | | |
| | TAGCACACCCATTCCAGGGGTGCGC | | |
| | CGTCCTGCACGTTTAGGGTTATA | | |
| | TAGTAAGGCTACACAACAAGTA | | |
| 625339_25353838_333048_ | AGGGTTTTCATATGTAATTAGCTGT | 120 | 65 |
| HPV31REF.1_1_81 | TTTGGAGCACTAAGAAACGTTGGAT | | |
| | CAATAACTTTTACTTGTTGTGTAGC | | |
| | CTTACTATATAACCCTAAACGTG | | |
| | CAGGACGGCGCACCCCTGGAAT | | |
| 625339_25353838_333048_ | AAAGTTATTGATCCAACGTTTCTTA | 120 | 65 |
| HPV31REF.1_1_82 | GTGCTCCAAAACAGCTAATTACATA | | |
| | TGAAAACCCTGCCTATGAAACTGTA | | |
| | AATGCTGAAGAATCTTTATACTTT | | |
| | TCCAATACATCGCATAATATA | | |
| 625339_25353838_333048_ | ACGTGAGGTAAGGGCAGGCCTATGT | 120 | 65 |
| HPV31REF.1_1_83 | AATGCTATAATATCTAGAAAGTCGG | | |
| | GATCAGGGGCTATATTATGCGATGT | | |
| | ATTGGAAAAGTATAAAGATTCT | | |
| | TCAGCATTTACAGTTTCATAGGC | | |
| 625339_25353838_333048_ | GCCCCTGATCCCGACTTTCTAGATA | 120 | 65 |
| HPV31REF.1_1_84 | TTATAGCATTACATAGGCCTGCCCT | | |
| | TACCTCACGTAGGAACACTGTTAGA | | |
| | TATAGTAGACTAGGTAATAAAC | | |
| | AAACTTTGCGCACTCGTAGTGGT | | |
| 625339_25353838_333048_ | ACCTGCAGGATTAATACTACTAATA | 120 | 65 |
| HPV31REF.1_1_85 | TCATAATAATAATGCACCCTTGCAC | | |
| | CAATAGTAGCACCACTACGAGTGCG | | |
| | CAAAGTTTGTTTATTACCTAGTC | | |
| | TACTATATCTAACAGTGTTCCT | | |
| 625339_25353838_333048_ | GCTACTATTGGTGCAAGGGTGCatt | 120 | 65 |
| HPV31REF.1_1_86 | attattatgatattagtagtattaA | | |
| | TCCTGCAGGTGAAAGTATTGAAATG | | |
| | CAACCTTTAGGGGCGTCTGCAACTA | | |
| | CTACTTC | | |
| | TACTTTAAATGAT | | |
| 625339_25353838_333048_ | ATTATGTGTGGCAGGTGTATCCACA | 120 | 65 |
| HPV31REF.1_1_87 | GTAAAATCAGTGTCTGCATAAATGT | | |
| | CATATAAGCCATCATTTAAAGTAGA | | |
| | AGTAGTAGTTGCAGACGCCCCT | | |
| | AAAGGTTGCATTTCAATACTTTC | | |
| 625339_25353838_333048_ | GGCTTATATGACATTTATGCAGACA | 120 | 65 |
| HPV31REF.1_1_88 | CTGATTTTACTGTGGATACACCTGC | | |
| | CACACATAATGTTTCCCCTTCTACT | | |
| | GCTGTACAGTCCACATCTGCTGT | | |
| | GTCTGCCTATGTACCTACAAAT | | |
| 625339_25353838_333048_ | AGGTACATCAGGCCCAGAAAATATG | 120 | 65 |
| HPV31REF.1_1_89 | GGAATGTCAAAACCTGTACTTAGTG | | |
| | GCACAGTGGTATTTGTAGGTACATA | | |
| | GGCAGACACAGCAGATGTGGA | | |
| | CTGTACAGCAGTAGAAGGGGAAAC | | |
| 625339_25353838_333048_ | ACCACTGTGCCACTAAGTACAGGTT | 120 | 65 |
| HPV31REF.1_1_90 | TTGACATTCCCATATTTTCTGGGCC | | |
| | TGATGTACCTATAGAGCATGCACCT | | |
| | ACACAGGTTTTCCCATTTCCTTT | | |
| | GGCCCCTACAACGCCACAAGTG | | |
| 625339_25353838_333048_ | ACGTTTTAACATATAATAACTAGGG | 120 | 66 |
| HPV31REF.1_1_91 | TGCAAATAAAAATCACCCCCATCAA | | |
| | CAAAAATAGACACTTGTGGCGTTGT | | |
| | AGGGGCCAAAGGAAATGGGAAA | | |
| | ACCTGTGTAGGTGCATGCTCTAT | | |
| 625339_25353838_333048_ | TCTATTTTTGTTGATGGGGGTGATT | 120 | 66 |
| HPV31REF.1_1_92 | TTTATTTGCACCCTAGTTATTATAT | | |
| | GTTAAAACGTCGACGTAAACGTGTA | | |
| | TCATATTTTTTTACAGATGTCTCT | | |
| | GTGGCGGCCTAGCGAGGCTAC | | |
| 625339_25353838_333048_ | CGTGTTACATATTCATCCGTGCTTA | 120 | 66 |
| HPV31REF.1_1_93 | CAACTTTAGACACTGGGACAGGTGG | | |
| | TAAGTAGACAGTAGCCTCGCTAGGC | | |
| | CGCCACAGAGACATCTGTAAA | | |
| | AAAATATGATACACGTTTACGTCG | | |
| 625339_25353838_333048_ | TGTCTACTTACCACCTGTCCCAGTG | 120 | 66 |
| HPV31REF.1_1_94 | TCTAAAGTTGTAAGCACGGATGAAT | | |
| | ATGTAACACGAACCAACATATATTA | | |
| | TCACGCAGGCAGTGCTAGGCTG | | |
| | CTTACAGTAGGCCATCCATATTA | | |
| 625339_25353838_333048_ | TGTAATCCTGACACCTTTGGTACAA | 120 | 66 |
| HPV31REF.1_1_95 | CTATTTTTTTAGGATTGTCAGATTT | | |
| | AGGTATGGAATAATATGGATGGCCT | | |
| | ACTGTAAGCAGCCTAGCACTGCC | | |
| | TGCGTGATAATATATGTTGGTT | | |
| 625339_25353838_333048_ | TTCCATACCTAAATCTGACAATCCT | 120 | 66 |
| HPV31REF.1_1_96 | AAAAAAATAGTTGTACCAAAGGTGT | | |
| | CAGGATTACAATATAGGGTATTTAG | | |
| | GGTTCGTTTACCAGATCCAAACA | | |
| | AATTTGGATTTCCTGATACATC | | |
| 625339_25353838_333048_ | CGACCTACCTCTAAACCAACACAGG | 120 | 66 |
| HPV31REF.1_1_97 | CCCAAACTAAGCGTTGAGTTTCAGG | | |
| | ATTATAAAAAGATGTATCAGGAAAT | | |
| | CCAAATTTGTTTGGATCTGGTA | | |
| | AACGAACCCTAAATACCCTATAT | | |
| 625339_25353838_333048_ | TTTTTATAATCCTGAAACTCAACGC | 120 | 66 |
| HPV31REF.1_1_98 | TTAGTTTGGGCCTGTGTTGGTTTAG | | |
| | AGGTAGGTCGCGGGCAGCCATTAGG | | |
| | TGTAGGTATTAGTGGTCATCCA | | |
| | TTATTAAATAAATTTGATGACAC | | |
| 625339_25353838_333048_ | ATTGATATACATTCCCTATTATCAG | 120 | 66 |
| HPV31REF.1_1_99 | TGCCAGGACCACCGGCATATCTATT | | |
| | AGAGTTTTCAGTGTCATCAAATTTA | | |
| | TTTAATAATGGATGACCACTAATA | | |
| | CCTACACCTAATGGCTGCCCG | | |
| 625339_25353838_333048_ | TGAAAACTCTAATAGATATGCCGGT | 120 | 66 |
| HPV31REF.1_1_100 | GGTCCTGGCACTGATAATAGGGAAT | | |
| | GTATATCAATGGATTATAAACAAAC | | |
| | ACAACTGTGTTTACTTGGTTGCA | | |
| | AACCACCTATTGGAGAGCATTG | | |
| 625339_25353838_333048_ | TCTAATGGAGGACAATCACCAGGGG | 120 | 67 |
| HPV31REF.1_1_101 | TAATAGCATTGTTACTACAAGGACT | | |
| | ACCTTTACCCCAATGCTCTCCAATA | | |
| | GGTGGTTTGCAACCAAGTAAAC | | |
| | ACAGTTGTGTTTGTTTATAATCC | | |
| 625339_25353838_333048_ | GGGTAAAGGTAGTCCTTGTAGTAAC | 120 | 67 |
| HPV31REF.1_1_102 | AATGCTATTACCCCTGGTGATTGTC | | |
| | CTCCATTAGAATTAAAAAATTCAGT | | |
| | TATACAAGATGGGGATATGGTTG | | |
| | ATACAGGCTTTGGAGCTATGGA | | |
| 625339_25353838_333048_ | CAAATAGAATTACAAATGTCCAAAG | 120 | 67 |
| HPV31REF.1_1_103 | GAACATTACTTTTAGTGTCTTGTAA | | |
| | AGCAGTAAAATCCATAGCTCCAAAG | | |
| | CCTGTATCAACCATATCCCCATC | | |
| | TTGTATAACTGAATTTTTT | | |
| | AAT | | |
| 625339_25353838_333048_ | TTTTACTGCTTTACAAGACACTAAA | 120 | 67 |
| HPV31REF.1_1_104 | AGTAATGTTCCTTTGGACATTTGTA | | |
| | ATTCTATTTGTAAATATCCAGATTA | | |
| | TCTTAAAATGGTTGCTGAGCCATAT | | |
| | GGCGATACATTATTTTTTTA | | |
| 625339_25353838_333048_ | TCACCAACCGTGCCTGATCTATTAA | 120 | 67 |
| HPV31REF.1_1_105 | AAAAATGCCTTACAAACATTTGTTC | | |
| | CCTACGTAAATAAAAAAATAATGTA | | |
| | TCGCCATATGGCTCAGCAACCAT | | |
| | TTTAAGATAATCTGGATATTTA | | |
| 625339_25353838_333048_ | TTTACGTAGGGAACAAATGTTTGTA | 120 | 67 |
| HPV31REF.1_1_106 | AGGCATTTTTTTAATAGATCAGGCA | | |
| | CGGTTGGTGAATCGGTCCCTACTGA | | |
| | CTTATATATTAAAGGCTCCGGTT | | |
| | CAACAGCTACTTTAGCTAACAG | | |
| 625339_25353838_333048_ | TTATTAAAAATTTGTGCATCTGAAG | 120 | 67 |
| HPV31REF.1_1_107 | TAACCATGGAGCCGCTAGGTGTAGG | | |
| | AAAGTATGTACTGTTAGCTAAAGTA | | |
| | GCTGTTGAACCGGAGCCTTTAA | | |
| | TATATAAGTCAGTAGGGACCGAT | | |
| 625339_25353838_333048_ | TACATACTTTCCTACACCTAGCGGC | 120 | 67 |
| HPV31REF.1_1_108 | TCCATGGTTACTTCAGATGCACAAA | | |
| | TTTTTAATAAACCATATTGGATGCA | | |
| | ACGTGCTCAGGGACACAATAATG | | |
| | GTATTTGTTGGGGCAATCAGTT | | |
| 625339_25353838_333048_ | GCAATTGCAGCACAAACAGACATAT | 120 | 67 |
| HPV31REF.1_1_109 | TGGTACTACGTGTGGTATCTACCAC | | |
| | AGTAACAAATAACTGATTGCCCCAA | | |
| | CAAATACCATTATTGTGTCCCT | | |
| | GAGCACGTTGCATCCAATATGGT | | |
| 625339_25353838_333048_ | ATTTGTTACTGTGGTAGATACCACA | 120 | 67 |
| HPV31REF.1_1_110 | CGTAGTACCAATATGTCTGTTTGTG | | |
| | CTGCAATTGCAAACAGTGATACTAC | | |
| | ATTTAAAAGTAGTAATTTTAAAGA | | |
| | GTATTTAAGACATGGTGAGGA | | |
| 625339_25353838_333048_ | GTCATTATGTCTGCAGATAATGTTA | 120 | 68 |
| HPV31REF.1_1_111 | TTTTGCATAACTGAAATATAAATTG | | |
| | TAAATCAAATTCCTCACCATGTCTT | | |
| | AAATACTCTTTAAAATTACTACTTT | | |
| | T | | |
| | AAATGTAGTATCACTGTTT | | |
| 625339_25353838_333048_ | ATTTGATTTACAATTTATATTTCAG | 120 | 68 |
| HPV31REF.1_1_112 | TTATGCAAAATAACATTATCTGCAG | | |
| | ACATAATGACATATATTCACAGTAT | | |
| | GAATCCTGCTATTTTGGAAGATTGG | | |
| | AATTTTGGATTGACCACACC | | |
| 625339_25353838_333048_ | TGACATGTAATGGCCTGTGAGGTGA | 120 | 68 |
| HPV31REF.1_1_113 | CAAACCTATAGGTATCCTCCAAAGA | | |
| | ACCTGAGGGAGGTGTGGTCAATCCA | | |
| | AAATTCCAATCTTCCAAAATAG | | |
| | CAGGATTCATACTGTGAATATAT | | |
| 625339_25353838_333048_ | TCCCTCAGGTTCTTTGGAGGATACC | 120 | 68 |
| HPV31REF.1_1_114 | TATAGGTTTGTCACCTCACAGGCCA | | |
| | TTACATGTCAAAAAACTGCCCCCCA | | |
| | AAAGCCCAAGGAAGATCCATTT | | |
| | AAAGATTATGTATTTTGGGAGGT | | |
| 625339_25353838_333048_ | AAAAATTTGCGACCCAGTGGAAACT | 120 | 68 |
| HPV31REF.1_1_115 | GATCTAAATCTGCAGAAAACTTTTC | | |
| | TTTTAAATTAACCTCCCAAAATACA | | |
| | TAATCTTTAAATGGATCTTCCTTG | | |
| | GGCTTTTGGGGGGCAGTTTTT | | |
| 625339_25353838_333048_ | TAATTTAAAAGAAAAGTTTTCTGCA | 120 | 68 |
| HPV31REF.1_1_116 | GATTTAGATCAGTTTCCACTGGGTC | | |
| | GCAAATTTTTATTACAGGCAGGATA | | |
| | TAGGGCACGTCCTAAATTTAAAG | | |
| | CAGGTAAACGTAGTGCACCCTC | | |
| 625339_25353838_333048_ | TACATACACATCCATTACTTTTTAG | 120 | 68 |
| HPV31REF.1_1_117 | TTTTTTTACGTTTTGCTGGTGTAGT | | |
| | GGTAGATGCTGAGGGTGCACTACGT | | |
| | TTACCTGCTTTAAATTTAGGACGT | | |
| | GCCCTATATCCTGCCTGTAAT | | |
| 625339_25353838_333048_ | AGCATCTACCACTACACCAGCAAAA | 120 | 68 |
| HPV31REF.1_1_118 | CGTAAAAAAACTAAAAAGTAAtgga | | |
| | tgtgtatgtaatacatgtgtctgta | | |
| | tgtgtatgtgcttgtgctgtattgt | | |
| | atatgtgtgtgtttgtgtgt | | |
| 625339_25353838_333048_ | AACATACTATACATATATACAAGTA | 120 | 68 |
| HPV31REF.1_1_119 | CACACGCATACATAAACATACATAT | | |
| | ACCATATATAACACACAAACACACA | | |
| | CATATACAATACAGCACAAGCACA | | |
| | TACACATACAGACACATGTAT | | |
| 625339_25353838_333048_ | tatatatggtatatgtatgtttatg | 120 | 68 |
| HPV31REF.1_1_120 | tatgcgtgtgtacttgtatatatgt | | |
| | atagtatgttatgtgtgtatgtatg | | |
| | ctatgtatgttaataaatatgtgta | | |
| | tacctgtgtgtgttgtgtat | | |
| 625339_25353838_333048_ | GGAACAATAGTTTATAAAATAGTAA | 120 | 69 |
| HPV31REF.1_1_121 | TAGTATGTTACTAATAGGGTGTATA | | |
| | TAAGGACAACATACACAACACACAC | | |
| | AGGTATACACATATTTATTAACAT | | |
| | ACATAGCATACATACACACAT | | |
| 625339_25353838_333048_ | GTTGTCCTTATATACACCCTATTAG | 120 | 69 |
| HPV31REF.1_1_122 | TAACATACTATTACTATTTTATAAA | | |
| | CTATTGTTCCTACTTGTTCCTACTT | | |
| | GTTCCTGCTCCTCCCAATAGTCATG | | |
| | TACTTATTTCTGCCTATAAT | | |
| 625339_25353838_333048_ | GTAGCTTTAGTTGCAAAAAACGGAC | 120 | 69 |
| HPV31REF.1_1_123 | CGGGTGTACAACTTTTACTATGGCG | | |
| | TGACACCTAAATTATAGGCAGAAAT | | |
| | AAGTACATGACTATTGGGAGGA | | |
| | GCAGGAACAAGTAGGAACAAGTA | | |
| 625339_25353838_333048_ | TTAGGTGTCACGCCATAGTAAAAGT | 120 | 69 |
| HPV31REF.1_1_124 | TGTACACCCGGTCCGTTTTTTGCAA | | |
| | CTAAAGCTACTCCATTTTGATTTTA | | |
| | TGCAGCCATTTTAAATCCCTAAC | | |
| | CGTTTTCGGTTGCATTGTTTAA | | |
| 625339_25353838_333048_ | TAGTATTCAGGAAACCAAAAACCGC | 120 | 69 |
| HPV31REF.1_1_125 | AGAACTACTGCATCAGCATAGTTGT | | |
| | ACTAGCATGTTTAAACAATGCAACC | | |
| | GAAAACGGTTAGGGATTTAAAA | | |
| | TGGCTGCATAAAATCAAAATGGA | | |
| 625339_25353838_333048_ | ACATGCTAGTACAACTATGCTGATG | 120 | 69 |
| HPV31REF.1_1_126 | CAGTAGTTCTGCGGTTTTTGGTTTC | | |
| | CTGAATACTAGTTTTTGCCAACATT | | |
| | CTGGCTTGTAGTTTCCTGCCTAA | | |
| | CACACCTTGCCAACATATAATC | | |
| 625339_25353838_333048_ | GTTGAACTACAGTTGTATTTAAACA | 120 | 69 |
| HPV31REF.1_1_127 | AACATGATTCATAGTATAATTGCAA | | |
| | AGTTGGACTGGATTATATGTTGGCA | | |
| | AGGTGTGTTAGGCAGGAAACTAC | | |
| | AAGCCAGAATGTTGGCAAAAAC | | |
| 625339_25353838_333048_ | CAGTCCAACTTTGCAATTATACTAT | 120 | 69 |
| HPV31REF.1_1_128 | GAATCATGTTTGTTTAAATACAACT | | |
| | GTAGTTCAACTATGTGTCATGCACA | | |
| | TATATTATATTATCCTACACACCTT | | |
| | AAACTGCTTTTAGGCACATA | | |
| 625339_25353838_333048_ | GGCAGTTTAAACATGTGCAAAAGCC | 120 | 69 |
| HPV31REF.1_1_129 | AGCACTGCAATCAAGGATATAGATA | | |
| | ATCTACAAAATATGTGCCTAAAAGC | | |
| | AGTTTAAGGTGTGTAGGATAAT | | |
| | ATAATATATGTGCATGACACATA | | |
| 625339_25353838_333048_ | TTTTGTAGATTATCTATATCCTTGA | 120 | 69 |
| HPV31REF.1_1_130 | TTGCAGTGCTGGCTTTTGCACATGT | | |
| | TTAAACTGCCAAGGTTGTGTCATGC | | |
| | ATTATAAATAAGTTGTATGTTACT | | |
| | CATATAATTAATTGCATATAG | | |
| 625339_25353838_333048_ | AGTATAAAAAGAACAATTGCTTGTA | 120 | 70 |
| HPV31REF.1_1_131 | AAACTGTAACCGAAAACGGTGTAAT | | |
| | ACCTATATGCAATTAATTATATGAG | | |
| | TAACATACAACTTATTTATAATGC | | |
| | ATGACACAACCTT | | |

| HPV33_DoubleStrand_ | | | SEQ |
|---|---|---|---|
| SequenceName | Sequence | Length | ID#: |
| 625339_25353839_333049_ | GTAAACTATAATGCCAAGTTTTAAA | 120 | 701 |
| HPV33REF.1_1_1 | AAAGTAGGGTGTAACCGAAAGCGGT | | |
| | TCAACCGAAAACGGTGCATATATAA | | |
| | AGCAAACATTTTGCAGTAAG | | |
| | GTACTGCACGACTATGTTTCAAGAC | | |
| 625339_25353839_333049_ | GTGTATAGTTGTCTCCAATGCTTGG | 120 | 702 |
| HPV33REF.1_1_2 | CACAAATCATGCAATGTTCGTGGTT | | |
| | TTTCCTCAGTGTCTTGAAACATAGT | | |
| | CGTGCAGTACCTTACTGCAA | | |
| | AATGTTTGCTTTATATATGCACCGT | | |
| 625339_25353839_333049_ | ACTGAGGAAAAACCACGAACATTGC | 120 | 703 |
| HPV33REF.1_1_3 | ATGATTTGTGCCAAGCATTGGAGAC | | |
| | AACTATACACAACATTGAACTACAG | | |
| | TGCGTGGAATGCAAAAAAC | | |
| | CTTTGCAACGATCTGAGGTATATGA | | |
| | T | | |
| 625339_25353839_333049_ | TTTACATATTCCAAATGGATTTCCC | 120 | 704 |
| HPV33REF.1_1_4 | TCTCTATATACAACTGTTAAATCTG | | |
| | CAAATGCAAAATCATATACCTCAGA | | |
| | TCGTTGCAAAGGTTTTTTGCAT | | |
| | TCCACGCACTGTAGTTCAATGTT | | |
| 625339_25353839_333049_ | TTTGCATTTGCAGATTTAACAGTTG | 120 | 705 |
| HPV33REF.1_1_5 | TATATAGAGAGGGAAATCCATTTGG | | |
| | AATATGTAAACTGTGTTTGCGGTTC | | |
| | TTATCTAAAATTAGTGAATAT | | |
| | AGACATTATAATTATTCTGTATAT | | |
| 625339_25353839_333049_ | AATACACCTAATTAATATTTCATTT | 120 | 706 |
| HPV33REF.1_1_6 | AAAGGTTTTTTAACTGTTTGTTCTA | | |
| | ATGTATTTCCATATACAGAATAATT | | |
| | ATAATGTCTATATTCACTAATTTT | | |
| | AGATAAGAACCGCAAACACAG | | |
| 625339_25353839_333049_ | GGAAATACATTAGAACAAACAGTTA | 120 | 707 |
| HPV33REF.1_1_7 | AAAAACCTTTAAATGAAATATTAAT | | |
| | TAGGTGTATTATATGTCAAAGACCT | | |
| | TTGTGTCCTCAAGAAAAAAAAC | | |
| | GACATGTGGATTTAAACAAACGA | | |
| 625339_25353839_333049_ | ACGTCGGGACCTCCAACACGCCGCA | 120 | 708 |
| HPV33REF.1_1_8 | CAGCGCCCTGCCCAACGACCCGAAA | | |
| | TATTATGAAATCGTTTGTTTAAATC | | |
| | CACATGTCGTTTTTTTTCTT | | |
| | GAGGACACAAAGGTCTTTGACATAT | | |
| 625339_25353839_333049_ | TTTCATAATATTTCGGGTCGTTGGG | 120 | 709 |
| HPV33REF.1_1_9 | CAGGGCGCTGTGCGGCGTGTTGGAG | | |
| | GTCCCGACGTAGAGAAACTGCACTG | | |
| | TGACGTGTAAAAACGCCA | | |
| | TGAGAGGACACAAGCCAACGTTAAA | | |
| | GG | | |
| 625339_25353839_333049_ | CACTTAATTGCTCATAGCAGTATAG | 120 | 710 |
| HPV33REF.1_1_10 | GTCAGTTGGTTCAGGATATAAATCT | | |
| | AAAACATATTCCTTTAACGTTGGCT | | |
| | TGTGTCCTCTCATGGCGTTTT | | |
| | TACACGTCACAGTGCAGTTTCTCT | | |
| 625339_25353839_333049_ | AATATGTTTTAGATTTATATCCTGA | 120 | 711 |
| HPV33REF.1_1_11 | ACCAACTGACCTATACTGCTATGAG | | |
| | CAATTAAGTGACAGCTCAGATGAGG | | |
| | ATGAAGGCTTGGACCGGCCA | | |
| | GATGGACAAGCACAACCAGCCACAG | | |
| 625339_25353839_333049_ | TGACACATAAACGAACTGTGGTGTT | 120 | 712 |
| HPV33REF.1_1_12 | ACAAGTGTGACAACAGGTTACAATG | | |
| | TAGTAATCAGCTGTGGCTGGTTGTG | | |
| | CTTGTCCATCTGGCCGGTC | | |
| | CAAGCCTTCATCCTCATCTGAGCTG | | |
| | T | | |
| 625339_25353839_333049_ | CTGATTACTACATTGTAACCTGTTG | 120 | 713 |
| HPV33REF.1_1_13 | TCACACTTGTAACACCACAGTTCGT | | |
| | TTATGTGTCAACAGTACAGCAAGTG | | |
| | ACCTACGAACCATACAGCAA | | |
| | CTACTTATGGGCACAGTGAATATTG | | |
| 625339_25353839_333049_ | TTGTACCTTCAGGATCGGCCATTGT | 120 | 714 |
| HPV33REF.1_1_14 | AGATGATGTTTATTGTTGTGCACAG | | |
| | GTAGGGCACACAATATTCACTGTGC | | |
| | CCATAAGTAGTTGCTGTATG | | |
| | GTTCGTAGGTCACTTGCTGTACTGT | | |
| 625339_25353839_333049_ | TGTGCCCTACCTGTGCACAACAATA | 120 | 715 |
| HPV33REF.1_1_15 | AACATCATCTACAATGGCCGATCCT | | |
| | GAAGGTACAAATGGGGCTGGGATGG | | |
| | GGTGTACTGGTTGGTTTG | | |
| | AGGTAGAAGCAGTCATAGAGAGAAG | | |
| | AA | | |
| 625339_25353839_333049_ | GTAAATCCGTGCCACTGTCATCTGC | 120 | 716 |
| HPV33REF.1_1_16 | TGTTTCATCCTCATCTTCTGAAATA | | |
| | TTATCTCCTGTTCTTCTCTCTATGA | | |
| | CTGCTTCTACCTCAAACCAAC | | |
| | GAGTACACCCCATCCCAGCCCCAT | | |
| 625339_25353839_333049_ | CAGGAGATAATATTTCAGAAGATGA | 120 | 717 |
| HPV33REF.1_1_17 | GGATGAAACAGCAGATGACAGTGGC | | |
| | ACGGATTTACTAGAGTTTATAGATG | | |
| | ATTCTATGGAAAATAGTATA | | |
| | CAGGCAGACACAGAGGCAGCCCGGG | | |
| 625339_25353839_333049_ | TTCGTTTTAGTGCACACACAGCATT | 120 | 718 |
| HPV33REF.1_1_18 | TAAATCATCCTCCCCTTCCTGTATA | | |
| | TTAAACAATGCCCGGGCTGCCTCTG | | |
| | TGTCTGCCTGTATACTATTTT | | |
| | CCATAGAATCATCTATAAACTCTA | | |
| 625339_25353839_333049_ | CATTGTTTAATATACAGGAAGGGGA | 120 | 719 |
| HPV33REF.1_1_19 | GGATGATTTAAATGCTGTGTGTGCA | | |
| | CTAAAACGAAAGTTTGCCGCATGTT | | |
| | CACAAAGTGCTGCGGAGGA | | |
| | CGTTGTTGATCGTGCTGCAAACCCG | | |
| | T | | |
| 625339_25353839_333049_ | GCTCATCTATTTTTCGTTTTCTGTA | 120 | 720 |
| HPV33REF.1_1_20 | TGTGCATTCTTTATTTTTATTAATA | | |
| | GACGTTCTACACGGGTTTGCAGCAC | | |
| | GATCAACAACGTCCTCCGCAG | | |
| | CACTTTGTGAACATGCGGCAAACT | | |
| 625339_25353839_333049_ | GTAGAACGTCTATTAATAAAAATAA | 120 | 721 |
| HPV33REF.1_1_21 | AGAATGCACATACAGAAAACGAAAA | | |
| | ATAGATGAGCTAGAAGACAGCGGAT | | |
| | ATGGCAATACTGAAGTGGAA | | |
| | ACTCAGCAGATGGTACAACAGGTAG | | |
| 625339_25353839_333049_ | AATCATCCCCCACCCCACTAGATTC | 120 | 722 |
| HPV33REF.1_1_22 | TAAGTCATTTAAGTTTGTGTCGCCA | | |
| | TTTTGACTTTCTACCTGTTGTACCA | | |
| | TCTGCTGAGTTTCCACTTCAG | | |
| | TATTGCCATATCCGCTGTCTTCTA | | |
| 625339_25353839_333049_ | AAAGTCAAAATGGCGACACAAACTT | 120 | 723 |
| HPV33REF.1_1_23 | AAATGACTTAGAATCTAGTGGGGTG | | |
| | GGGGATGATTCAGAAGTAAGCTGTG | | |
| | AGACAAATGTAGATAGCTG | | |
| | TGAAAATGTTACGTTGCAGGAAATT | | |
| | A | | |
| 625339_25353839_333049_ | AGGCCTCTTTAAATTTATATAATAT | 120 | 724 |
| HPV33REF.1_1_24 | ATTTGCTTTTGTATTACTACTATGT | | |
| | AGAACATTACTAATTTCCTGCAACG | | |
| | TAACATTTTCACAGCTATCTAC | | |
| | ATTTGTCTCACAGCTTACTTCTG | | |
| 625339_25353839_333049_ | GTAATGTTCTACATAGTAGTAATAC | | |
| HPV33REF.1_1_25 | AAAAGCAAATATATTATATAAATTT | 120 | 725 |
| | AAAGAGGCCTATGGAATAAGTTTTA | | |
| | TGGAATTAGTAAGACCATTTAA | | |
| | AAGTGATAAAACAAGCTGTACAG | | |
| 625339_25353839_333049_ | TTAATACTTTTAAACTTTCTGCTAC | | |
| HPV33REF.1_1_26 | TGATGGACTAATTCCATATCCTGTT | 120 | 726 |
| | ATACACCAATCTGTACAGCTTGTTT | | |
| | TATCACTTTTAAATGGTCTTAC | | |
| | TAATTCCATAAAACTTATTCCAT | | |
| 625339_25353839_333049_ | ATTGGTGTATAACAGGATATGGAAT | | |
| HPV33REF.1_1_27 | TAGTCCATCAGTAGCAGAAAGTTTA | 120 | 727 |
| | AAAGTATTAATTAAACAGCATAGTT | | |
| | TGTATACTCATTTACAATGTT | | |
| | TAACTTGCGATAGAGGAATAATAA | | |
| 625339_25353839_333049_ | TCATTAGTTTTGCTACTGTTAACCT | | |
| HPV33REF.1_1_28 | GTTTTTGCTACACCTAAATCTAATT | 120 | 728 |
| | AACAATAATATTATTATTCCTCTAT | | |
| | CGCAAGTTAAACATTGTAAATGA | | |
| | GTATACAAACTATGCTGTTTAA | | |
| 625339_25353839_333049_ | TATTATTGTTAATTAGATTTAGGTG | | |
| HPV33REF.1_1_29 | TAGCAAAAACAGGTTAACAGTAGCA | 120 | 729 |
| | AAACTAATGAGTAATTTATTATCAA | | |
| | TACCTGAAACATGTATGGTTAT | | |
| | AGAGCCACCAAAATTACGGAGCC | | |
| 625339_25353839_333049_ | TACCTTGTACATCACTAATGTTTGA | 120 | 730 |
| HPV33REF.1_1_30 | CATTGCTGTTCTAAACCAATACAAT | | |
| | GCACATGTTTGGCTCCGTAATTTTG | | |
| | GTGGCTCTATAACCATACATG | | |
| | TTTCAGGTATTGATAATAAATTAC | | |
| 625339_25353839_333049_ | AAACATGTGCATTGTATTGGTTTAG | 120 | 731 |
| HPV33REF.1_1_31 | AACAGCAATGTCAAACATTAGTGAT | | |
| | GTACAAGGTACAACACCTGAATGGA | | |
| | TAGATAGACTAACTGTTTTA | | |
| | CAACATAGCTTTAATGATAATATAT | | |
| 625339_25353839_333049_ | TGTCACTATCGTCCGTTAACTCGTT | 120 | 732 |
| HPV33REF.1_1_32 | ATCATATGCCCACTGTACCATTTCA | | |
| | CTTAAATCAAATATATTATCATTAA | | |
| | AGCTATGTTGTAAAACAGTTAG | | |
| | TCTATCTATCCATTCAGGTGTTG | | |
| 625339_25353839_333049_ | TTGATTTAAGTGAAATGGTACAGTG | 120 | 733 |
| HPV33REF.1_1_33 | GGCATATGATAACGAGTTAACGGAC | | |
| | GATAGTGACATTGCATATTATTATG | | |
| | CACAACTTGCAGATTCAAAT | | |
| | AGTAATGCTGCTGCATTTTTAAAAA | | |
| 625339_25353839_333049_ | CTTTTTTATAATGTCTACACATTAT | 120 | 734 |
| HPV33REF.1_1_34 | TCCACAGTCCTTTACTATTTTTGCT | | |
| | TGTGAGTTACTTTTTAAAAATGCAG | | |
| | CAGCATTACTATTTGAATCTGC | | |
| | AAGTTGTGCATAATAATATGCAA | | |
| 625339_25353839_333049_ | GTAACTCACAAGCAAAAATAGTAAA | 120 | 735 |
| HPV33REF.1_1_35 | GGACTGTGGAATAATGTGTAGACAT | | |
| | TATAAAAAAGCAGAAAAACGTAAAA | | |
| | TGTCAATAGGACAATGGATA | | |
| | CAAAGTAGATGTGAAAAAACAAATG | | |
| 625339_25353839_333049_ | CTGTAAATTCAATGTTTTGATATCT | 120 | 736 |
| HPV33REF.1_1_36 | TAACAACTGTACTATTGGTCTCCAA | | |
| | TTTCCTCCATCATTTGTTTTTTCAC | | |
| | ATCTACTTTGTATCCATTGTCC | | |
| | TATTGACATTTTACGTTTTTCTG | | |
| 625339_25353839_333049_ | ATGGAGGAAATTGGAGACCAATAGT | 120 | 737 |
| HPV33REF.1_1_37 | ACAGTTGTTAAGATATCAAAACATT | | |
| | GAATTTACAGCATTTTTAGGTGCAT | | |
| | TTAAAAAGTTTTTAAAAGGTA | | |
| | TACCAAAAAAAAGCTGTATGCTAA | | |
| 625339_25353839_333049_ | TTAAAAACTGTATTAAACTCATTCC | 120 | 738 |
| HPV33REF.1_1_38 | AAAATATGACTTTCCTGTATTTGCT | | |
| | GGTCCACAAATTAGCATACAGCTTT | | |
| | TTTTTGGTATACCTTTTAAAAA | | |
| | CTTTTTAAATGCACCTAAAAATG | | |
| 625339_25353839_333049_ | TTTGTGGACCAGCAAATACAGGAAA | 120 | 739 |
| HPV33REF.1_1_39 | GTCATATTTTGGAATGAGTTTAATA | | |
| | CAGTTTTTAAAAGGGTGTGTTATAT | | |
| | CATGTGTAAATTCTAAAAGTC | | |
| | ACTTTTGGTTGCAGCCATTATCAG | | |
| 625339_25353839_333049_ | AATCATCTATATATGTCCAACTTAT | 120 | 740 |
| HPV33REF.1_1_40 | TGGCGTTACATCATCTATCATTCCT | | |
| | ATTTTTGCATCTGATAATGGCTGCA | | |
| | ACCAAAAGTGACTTTTAGAATT | | |
| | TACACATGATATAACACACCCTT | | |
| 625339_25353839_333049_ | ATGCAAAAATAGGAATGATAGATGA | 120 | 741 |
| HPV33REF.1_1_41 | TGTAACGCCAATAAGTTGGACATAT | | |
| | ATAGATGATTACATGAGAAATGCGT | | |
| | TAGATGGAAATGAAATTTCA | | |
| | ATAGATGTGAAACATAGGGCATTAG | | |
| 625339_25353839_333049_ | TAGAGTCTGTGCCTGCATTTGTATT | 120 | 742 |
| HPV33REF.1_1_42 | TGAGGTAAGAAGCAGTGGTGGACAT | | |
| | TTTAATTGCACTAATGCCCTATGTT | | |
| | TCACATCTATTGAAATTTCAT | | |
| | TTCCATCTAACGCATTTCTCATGT | | |
| 625339_25353839_333049_ | TGCAATTAAAATGTCCACCACTGCT | 120 | 743 |
| HPV33REF.1_1_43 | TCTTACCTCAAATACAAATGCAGGC | | |
| | ACAGACTCTAGATGGCCATATTTAC | | |
| | ATAGTAGATTAACAGTATTTG | | |
| | AATTTAAAAATCCATTCCCATTTG | | |
| 625339_25353839_333049_ | TTGAGAAAAAGGATTTCCAATTTTC | 120 | 744 |
| HPV33REF.1_1_44 | ATCATTTATTGCATACACTGGGTTA | | |
| | CCATTTTCATCAAATGGGAATGGAT | | |
| | TTTTAAATTCAAATACTGTTAA | | |
| | TCTACTATGTAAATATGGCCATC | | |
| 625339_25353839_333049_ | ATGAAAATGGTAACCCAGTGTATGC | 120 | 745 |
| HPV33REF.1_1_45 | AATAAATGATGAAAATTGGAAATCC | | |
| | TTTTTCTCAAGGACGTGGTGCAAAT | | |
| | TAGATTTAATAGAGGAAGAG | | |
| | GACAAGGAAAACCATGGAGGAAATA | | |
| 625339_25353839_333049_ | AGTTTTATCAGCTTCGTAAAGATCT | 120 | 746 |
| HPV33REF.1_1_46 | AGTATTTTCTCCTGCACTGCATTTA | | |
| | AACGTGCTGATATTTCCTCCATGGT | | |
| | TTTCCTTGTCCTCTTCCTCTA | | |
| | TTAAATCTAATTTGCACCACGTCC | | |
| 625339_25353839_333049_ | TCAGCACGTTTAAATGCAGTGCAGG | 120 | 747 |
| HPV33REF.1_1_47 | AGAAAATACTAGATCTTTACGAAGC | | |
| | TGATAAAACTGATTTACCATCACAA | | |
| | ATTGAACATTGGAAACTGAT | | |
| | ACGCATGGAGTGTGCTTTATTGTAT | | |
| 625339_25353839_333049_ | TGCTAACAAAGAAGGCACCACCTGG | 120 | 748 |
| HPV33REF.1_1_48 | TGGCATAAATGTGAAAATCCCATTT | | |
| | GTTTGGCTGTATACAATAAAGCACA | | |
| | CTCCATGCGTATCAGTTTCC | | |
| | AATGTTCAATTTGTGATGGTAAATC | | |
| 625339_25353839_333049_ | ACAGCCAAACAAATGGGATTTTCAC | 120 | 749 |
| HPV33REF.1_1_49 | ATTTATGCCACCAGGTGGTGCCTTC | | |
| | TTTGTTAGCATCAAAGACCAAAGCA | | |
| | TTTCAAGTAATTGAACTACA | | |
| | AATGGCATTAGAGACATTAAGTAAA | | |
| 625339_25353839_333049_ | ACAAAGCCACACCTCTAAGCTTGTT | 120 | 750 |
| HPV33REF.1_1_50 | TGTTGCAATGTCCATTGGCTTGTAC | | |
| | TATACTGTGATTTACTTAATGTCTC | | |
| | TAATGCCATTTGTAGTTCAAT | | |
| | TACTTGAAATGCTTTGGTCTTTGA | | |
| 625339_25353839_333049_ | TCACAGTATAGTACAAGCCAATGGA | 120 | 751 |
| HPV33REF.1_1_51 | CATTGCAACAAACAAGCTTAGAGGT | | |
| | GTGGCTTTGTGAACCACCAAAATGT | | |
| | TTTAAAAAACAAGGAGAAAC | | |
| | AGTAACTGTGCAATATGACAATGAC | | |
| 625339_25353839_333049_ | TGTATCTTCCTCTATAATATATATT | 120 | 752 |
| HPV33REF.1_1_52 | TCACCCCAGTTTGTATAATCCATTG | | |
| | TATTTTTTTTGTCATTGTCATATTG | | |
| | CACAGTTACTGTTTCTCCTTGTT | | |
| | TTTTAAAACATTTTGGTGGTTC | | |
| 625339_25353839_333049_ | AAAAAAAATACAATGGATTATACAA | 120 | 753 |
| HPV33REF.1_1_53 | ACTGGGGTGAAATATATATTATAGA | | |
| | GGAAGATACATGTACTATGGTTACA | | |
| | GGGAAAGTAGATTATATAGGT | | |
| | ATGTATTATATACATAACTGTGAA | | |
| 625339_25353839_333049_ | CCACATTTGTGTTTTAGAATACTTT | 120 | 754 |
| HPV33REF.1_1_54 | GCAGCATCCTCTTTAAAATATTTAA | | |
| | AATATACCTTTTCACAGTTATGTAT | | |
| | ATAATACATACCTATATAATCTA | | |
| | CTTTCCCTGTAACCATAGTACA | | |
| 625339_25353839_333049_ | AAGGTATATTTTAAATATTTTAAAG | 120 | 755 |
| HPV33REF.1_1_55 | AGGATGCTGCAAAGTATTCTAAAAC | | |
| | ACAAATGTGGGAAGTACATGTGGGT | | |
| | GGTCAGGTAATTGTTTGTCC | | |
| | TACGTCTATATCTAGCAACCAAATA | | |
| 625339_25353839_333049_ | CGCTGCTTGTGGTGGTCGGTTATCG | 120 | 756 |
| HPV33REF.1_1_56 | TTGTCTGTCTGTATGTCAGCAGTTT | | |
| | CAGTAGTGGATATTTGGTTGCTAGA | | |
| | TATAGACGTAGGACAAACA | | |
| | ATTACCTGACCACCCACATGTACTT | | |
| | C | | |
| 625339_25353839_333049_ | TCCACTACTGAAACTGCTGACATAC | 120 | 757 |
| HPV33REF.1_1_57 | AGACAGACAACGATAACCGACCACC | | |
| | ACAAGCAGCGGCCAAACGACGACGA | | |
| | CCTGCAGACACCACAGAC | | |
| | ACCGCCCAGCCCCTTACAAAGCTGT | | |
| | TC | | |
| 625339_25353839_333049_ | CTTGTTTGTGCAGTTAGTTGCAGTA | 120 | 758 |
| HPV33REF.1_1_58 | CGTGCTGTTCTATTGTCCAAGGCGG | | |
| | GGTCTGCACAGAACAGCTTTGTAAG | | |
| | GGGCTGGGCGGTGTCTGT | | |
| | GGTGTCTGCAGGTCGTCGTCGTTTG | | |
| | GC | | |
| 625339_25353839_333049_ | TGTGCAGACCCCGCCTTGGACAATA | 120 | 759 |
| HPV33REF.1_1_59 | GAACAGCACGTACTGCAACTAACTG | | |
| | CACAAACAAGCAGCGGACTGTGTGT | | |
| | AGTTCTAACGTTGCACCTA | | |
| | TAGTGCATTTAAAAGGTGAATCAAA | | |
| | T | | |
| 625339_25353839_333049_ | TGACATAGAACTATACAACTCTTTA | 120 | 760 |
| HPV33REF.1_1_60 | TAAGGTTTTAATCTGTATCTTAAAC | | |
| | ATTTTAAACTATTTGATTCACCTTT | | |
| | TAAATGCACTATAGGTGCAACG | | |
| | TTAGAACTACACACAGTCCGCTG | | |
| 625339_25353839_333049_ | AGTTTAAAATGTTTAAGATACAGAT | 120 | 761 |
| HPV33REF.1_1_61 | TAAAACCTTATAAAGAGTTGTATAG | | |
| | TTCTATGTCATCCACCTGGCATTGG | | |
| | ACCAGTGACAACAAAAATAGT | | |
| | AAAAATGGAATTGTAACTGTAACA | | |
| 625339_25353839_333049_ | CACAGTAGGTGGTATTTTTACGGTA | 120 | 762 |
| HPV33REF.1_1_62 | CCTAAAAACATTTGTTGTTGCTGTT | | |
| | CAGTTACAAATGTTACAGTTACAAT | | |
| | TCCATTTTTACTATTTTTGTTG | | |
| | TCACTGGTCCAATGCCAGGTGGA | | |
| 625339_25353839_333049_ | TTTGTAACTGAACAGCAACAACAAA | 120 | 763 |
| HPV33REF.1_1_63 | TGTTTTTAGGTACCGTAAAAATACC | | |
| | ACCTACTGTGCAAATAAGTACTGGA | | |
| | TTTATGACATTATAAGTGTAC | | |
| | ATCACAAGCCAATATGTGCTGCTA | | |
| 625339_25353839_333049_ | GATAAGCATAAAAACAATATAAAAC | 120 | 764 |
| HPV33REF.1_1_64 | ATAATACAAAAACAAATATCATGGT | | |
| | TATATACAATTAGCAGCACATATTG | | |
| | GCTTGTGATGTACACTTATAAT | | |
| | GTCATAAATCCAGTACTTATTTG | | |
| 625339_25353839_333049_ | ATTGTATATAACCATGATATTTGTT | 120 | 765 |
| HPV33REF.1_1_65 | TTTGtattatgttttatattgtttt | | |
| | tatgcttatccttattattaCGTCC | | |
| | TTTAATACTTTCCATTTCTACCTAT | | |
| | GCTTGGTTGCTGGTG | | |
| | TTGGT | | |
| 625339_25353839_333049_ | AACAACAAATAGCAAAAAAAAATTT | 120 | 766 |
| HPV33REF.1_1_66 | TTAAAGGAGATCCCACAAACACCCA | | |
| | AAGCAGCAATACCAACACCAGCAAC | | |
| | CAAGCATAGGTAGAAATGGA | | |
| | AAGTATTAAAGGACGTAATAATAAG | | |
| 625339_25353839_333049_ | ATTGCTGCTTTGGGTGTTTGTGGGA | 120 | 767 |
| HPV33REF.1_1_67 | TCTCCTTTAAAAATTTTTTTTTGCT | | |
| | ATTTGTTGTTTTTATATTTACCAAT | | |
| | GATGTGTATTAATTTTCATGCA | | |
| | CAGCATATGACACAACAAGAGTA | | |
| 625339_25353839_333049_ | ACAATGTTAAAACACCACCATGTGC | 120 | 768 |
| HPV33REF.1_1_68 | ACATATATACAAACAATATATACAT | | |
| | GTATATACATTACTCTTGTTGTGTC | | |
| | ATATGCTGTGCATGAAAATTAA | | |
| | TACACATCATTGGTAAATATAAA | | |
| 625339_25353839_333049_ | ATGTATATAGATGTATATATTGTTT | 120 | 769 |
| HPV33REF.1_1_69 | GTATATATGTGGAGATGGTGGTGTT | | |
| | TTAACATTGTTGTTGTTATTTTAGT | | |
| | TTTTTTTTTTTTGTATTAGTAAT | | |
| | AAATACCTTTATATTTTAGCAG | | |
| 625339_25353839_333049_ | TAGTTGTGTTGCAGATGCACGCTTG | 120 | 770 |
| HPV33REF.1_1_70 | CGCCTTGTAGATCGTTTGTGTCTCA | | |
| | TAATAATACACTGCTAAAATATAAA | | |
| | GGTATTTATTAGTAATACAAA | | |
| | AAAAAAAAAACTAAAATAACAACA | | |
| 625339_25353839_333049_ | TGTATTATTATGAGACACAAACGAT | 120 | 771 |
| HPV33REF.1_1_71 | CTACAAGGCGCAAGCGTGCATCTGC | | |
| | AACACAACTATACCAAACATGCAAG | | |
| | GCCACAGGCACCTGCCCAC | | |
| | CCGATGTTATTCCTAAAGTGGAAGG | | |
| | A | | |
| 625339_25353839_333049_ | TAAACCACCAAAAAAAACCCCTAAA | 120 | 772 |
| HPV33REF.1_1_72 | CTGCCATATTTAAGAATTTGATCTG | | |
| | CTATGGTACTTCCTTCCACTTTAGG | | |
| | AATAACATCGGGTGGGCAGG | | |
| | TGCCTGTGGCCTTGCATGTTTGGTA | | |
| 625339_25353839_333049_ | AGTACCATAGCAGATCAAATTCTTA | 120 | 773 |
| HPV33REF.1_1_73 | AATATGGCAGTTTAGGGGTTTTTTT | | |
| | TGGTGGTTTAGGTATTGGCACAGGC | | |
| | TCTGGTTCAGGTGGAAGGA | | |
| | CTGGCTATGTACCTATTGGTACTGA | | |
| | C | | |
| 625339_25353839_333049_ | AACAGTGTCTACAGTAACCGGAGGA | 120 | 774 |
| HPV33REF.1_1_74 | CGTATAGGCTGCAAGGGGATTGCAG | | |
| | CTGTAGGTGGGTCAGTACCAATAGG | | |
| | TACATAGCCAGTCCTTCC | | |
| | ACCTGAACCAGAGCCTGTGCCAATA | | |
| | CC | | |
| 625339_25353839_333049_ | CCACCTACAGCTGCAATCCCCTTGC | 120 | 775 |
| HPV33REF.1_1_75 | AGCCTATACGTCCTCCGGTTACTGT | | |
| | AGACACTGTTGGACCTTTAGACTCG | | |
| | TCTATAGTGTCATTAATAGA | | |
| | AGAAACAAGTTTTATAGAGGCAGGT | | |
| 625339_25353839_333049_ | AGTATCTGCAGATGTAGTAACATCA | 120 | 776 |
| HPV33REF.1_1_76 | AAACCTGATGGTGTAGGAATAGATG | | |
| | GGGCTGGTGCACCTGCCTCTATAAA | | |
| | ACTTGTTTCTTCTATTAATG | | |
| | ACACTATAGACGAGTCTAAAGGTCC | | |
| 625339_25353839_333049_ | GCACCAGCCCCATCTATTCCTACAC | 120 | 777 |
| HPV33REF.1_1_77 | CATCAGGTTTTGATGTTACTACATC | | |
| | TGCAGATACTACACCTGCAATTATT | | |
| | AATGTTTCATCTGTTGGGGA | | |
| | GTCATCTATTCAAACTATTTCTACA | | |
| 625339_25353839_333049_ | GGCTTCTGCAGGCGCTGGAGGGTGT | 120 | 778 |
| HPV33REF.1_1_78 | AGTACAGATGGTTCAGTAAATGTGG | | |
| | GATTTAAATGTGTAGAAATAGTTTG | | |
| | AATAGATGACTCCCCAACA | | |
| | GATGAAACATTAATAATTGCAGGTG | | |
| | T | | |
| 625339_25353839_333049_ | CATTTAAATCCCACATTTACTGAAC | 120 | 779 |
| HPV33REF.1_1_79 | CATCTGTACTACACCCTCCAGCGCC | | |
| | TGCAGAAGCCTCTGGACATTTTATA | | |
| | TTTTCTTCCCCTACTGTTAGC | | |
| | ACACAAAGTTATGAAAACATACCA | | |
| 625339_25353839_333049_ | TGGAATGGGCGTGCTTGATGTTACA | 120 | 780 |
| HPV33REF.1_1_80 | TTACTACTGTCTGTGGAAACAACAA | | |
| | AGGTATCCATTGGTATGTTTTCATA | | |
| | ACTTTGTGTGCTAACAGTAG | | |
| | GGGAAGAAAATATAAAATGTCCAGA | | |
| 625339_25353839_333049_ | ATGGATACCTTTGTTGTTTCCACAG | 120 | 781 |
| HPV33REF.1_1_81 | ACAGTAGTAATGTAACATCAAGCAC | | |
| | GCCCATTCCAGGGTCTCGCCCTGTG | | |
| | GCACGCCTTGGTTTATATA | | |
| | GTCGCAATACCCAACAGGTTAAGGT | | |
| | T | | |
| 625339_25353839_333049_ | AAATGCAGGATTATCATATGTTATA | 120 | 782 |
| HPV33REF.1_1_82 | AGTTTATGAGGCGATGTTAAAAAAG | | |
| | CAGGGTCAACAACCTTAACCTGTTG | | |
| | GGTATTGCGACTATATAAAC | | |
| | CAAGGCGTGCCACAGGGCGAGACCC | | |
| 625339_25353839_333049_ | GTTGACCCTGCTTTTTTAACATCGC | 120 | 783 |
| HPV33REF.1_1_83 | CTCATAAACTTATAACATATGATAA | | |
| | TCCTGCATTTGAAAGCTTTGACCCT | | |
| | GAAGACACATTACAATTTCAA | | |
| | CATAGTGATATATCACCTGCTCCT | | |
| 625339_25353839_333049_ | ATGTCTACGAGATGTAATAGCAGGC | 120 | 784 |
| HPV33REF.1_1_84 | CTATGTAATGCAATAATATCTAGAA | | |
| | AGTCAGGATCAGGAGCAGGTGATAT | | |
| | ATCACTATGTTGAAATTGTA | | |
| | ATGTGTCTTCAGGGTCAAAGCTTTC | | |
| 625339_25353839_333049_ | GATCCTGACTTTCTAGATATTATTG | 120 | 785 |
| HPV33REF.1_1_85 | CATTACATAGGCCTGCTATTACATC | | |
| | TCGTAGACATACTGTGCGTTTTAGT | | |
| | AGAGTAGGTCAAAAAGCCAC | | |
| | ACTTAAAACTCGCAGTGGTAAACAA | | |
| 625339_25353839_333049_ | GGTGTGGTCTAAAGGCACAATAGGA | 120 | 786 |
| HPV33REF.1_1_86 | CTTAAATCCTGATAATAATGTATTC | | |
| | TAGCTCCAATTTGTTTACCACTGCG | | |
| | AGTTTTAAGTGTGGCTTTTT | | |
| | GACCTACTCTACTAAAACGCACAGT | | |
| 625339_25353839_333049_ | ATTGGAGCTAGAATACATTATTATC | 120 | 787 |
| HPV33REF.1_1_87 | AGGATTTAAGTCCTATTGTGCCTTT | | |
| | AGACCACACCGTGCCAAATGAACAA | | |
| | TATGAATTACAGCCTTTACAT | | |
| | GATACTTCTACATCGTCTTATAGT | | |
| 625339_25353839_333049_ | CATTGGGGTGTGTACATTATCCACA | 120 | 788 |
| HPV33REF.1_1_88 | TCGTCAGCATAAACATCATACAAAC | | |
| | CATCATTAATACTATAAGACGATGT | | |
| | AGAAGTATCATGTAAAGGCT | | |
| | GTAATTCATATTGTTCATTTGGCAC | | |
| 625339_25353839_333049_ | ATTAATGATGGTTTGTATGATGTTT | 120 | 789 |
| HPV33REF.1_1_89 | ATGCTGACGATGTGGATAATGTACA | | |
| | CACCCCAATGCAACACTCATACAGT | | |
| | ACGTTTGCAACAACACGTAC | | |
| | CAGCAATGTGTCTATACCTTTAAAT | | |
| 625339_25353839_333049_ | TGTGGGAAATAAAGGGGAAGGTATA | 120 | 790 |
| HPV33REF.1_1_90 | TCAGGGCCAGACATAACAGGAGTAT | | |
| | CAAATCCTGTATTTAAAGGTATAGA | | |
| | CACATTGCTGGTACGTGTT | | |
| | GTTGCAAACGTACTGTATGAGTGTT | | |
| | G | | |
| 625339_25353839_333049_ | ACAGGATTTGATACTCCTGTTATGT | 120 | 791 |
| HPV33REF.1_1_91 | CTGGCCCTGATATACCTTCCCCTTT | | |
| | ATTTCCCACATCTAGCCCATTTGTT | | |
| | CCTATTTCGCCTTTTTTTCCT | | |
| | TTTGACACCATTGTTGTAGACGGT | | |
| 625339_25353839_333049_ | TGGAAAACGTTTACGCCTGCGACGT | 120 | 792 |
| HPV33REF.1_1_92 | AAAATAAAATAACTAGGATGTAAAA | | |
| | CAAAGTCAGCACCGTCTACAACAAT | | |
| | GGTGTCAAAAGGAAAAAAAG | | |
| | GCGAAATAGGAACAAATGGGCTAGA | | |
| 625339_25353839_333049_ | GCTGACTTTGTTTTACATCCTAGTT | 120 | 793 |
| HPV33REF.1_1_93 | ATTTTATTTTACGTCGCAGGCGTAA | | |
| | ACGTTTTCCATATTTTTTTACAGAT | | |
| | GTCCGTGTGGCGGCCTAGTGA | | |
| | GGCCACAGTGTACCTGCCTCCTGT | | |
| 625339_25353839_333049_ | TAATAATAAATGCTTGTGCGAGACA | 120 | 794 |
| HPV33REF.1_1_94 | CATATTCATCAGTGCTGACAACTTT | | |
| | AGATACAGGTACAGGAGGCAGGTAC | | |
| | ACTGTGGCCTCACTAGGCC | | |
| | GCCACACGGACATCTGTAAAAAAAT | | |
| | A | | |
| 625339_25353839_333049_ | ACCTGTATCTAAAGTTGTCAGCACT | 120 | 795 |
| HPV33REF.1_1_95 | GATGAATATGTGTCTCGCACAAGCA | | |
| | TTTATTATTATGCTGGTAGTTCCAG | | |
| | ACTTCTTGCTGTTGGCCATC | | |
| | CATATTTTTCTATTAAAAATCCTAC | | |
| 625339_25353839_333049_ | ACCCTAAAAACCCTATATTGCAAGC | 120 | 796 |
| HPV33REF.1_1_96 | CTGATACTTTGGGTACCAATAATTT | | |
| | TTTAGCGTTAGTAGGATTTTTAATA | | |
| | GAAAAATATGGATGGCCAACA | | |
| | GCAAGAAGTCTGGAACTACCAGCA | | |
| 625339_25353839_333049_ | TAACGCTAAAAAATTATTGGTACCC | 120 | 797 |
| HPV33REF.1_1_97 | AAAGTATCAGGCTTGCAATATAGGG | | |
| | TTTTTAGGGTCCGTTTACCAGATCC | | |
| | TAATAAATTTGGATTTCCTGA | | |
| | CACCTCCTTTTATAACCCTGATAC | | |
| 625339_25353839_333049_ | ACGCCTAATGGCTGCCCTCTACCTA | 120 | 798 |
| HPV33REF.1_1_98 | TTTCAAGGCCTACACATGCCCATAC | | |
| | TAATCGTTGTGTATCAGGGTTATAA | | |
| | AAGGAGGTGTCAGGAAATC | | |
| | CAAATTTATTAGGATCTGGTAAACG | | |
| | G | | |
| 625339_25353839_333049_ | ACAACGATTAGTATGGGCATGTGTA | 120 | 799 |
| HPV33REF.1_1_99 | GGCCTTGAAATAGGTAGAGGGCAGC | | |
| | CATTAGGCGTTGGCATAAGTGGTCA | | |
| | TCCTTTATTAAACAAATTT | | |
| | GATGACACTGAAACCGGTAACAAGT | | |
| | A | | |
| 625339_25353839_333049_ | TGTGTTTGTTTATAATCCATGGATA | 120 | 800 |
| HPV33REF.1_1_100 | AACATTCCCTATTATCAGCACCCGG | | |
| | TTGTCCAGGATACTTGTTACCGGTT | | |
| | TCAGTGTCATCAAATTTGTTT | | |
| | AATAAAGGATGACCACTTATGCCA | | |
| 625339_25353839_333049_ | TCCTGGACAACCGGGTGCTGATAAT | 120 | 801 |
| HPV33REF.1_1_101 | AGGGAATGTTTATCCATGGATTATA | | |
| | AACAAACACAGTTATGTTTACTTGG | | |
| | ATGTAAGCCTCCAACAGGG | | |
| | GAACATTGGGGTAAAGGTGTTGCTT | | |
| | G | | |
| 625339_25353839_333049_ | TCAATAATAGTATTTATAAGTTCTA | 120 | 802 |
| HPV33REF.1_1_102 | AAGGTGGACAATCATTGGCAGGTGC | | |
| | TGCATTAGTACAAGCAACACCTTTA | | |
| | CCCCAATGTTCCCCTGTTGG | | |
| | AGGCTTACATCCAAGTAAACATAAC | | |
| 625339_25353839_333049_ | TACTAATGCAGCACCTGCCAATGAT | 120 | 803 |
| HPV33REF.1_1_103 | TGTCCACCTTTAGAACTTATAAATA | | |
| | CTATTATTGAGGATGGTGATATGGT | | |
| | GGACACAGGATTTGGTTGCA | | |
| | TGGATTTTAAAACATTGCAGGCTAA | | |
| 625339_25353839_333049_ | TTTAAATAATCTGGATATTTGCATG | 120 | 804 |
| HPV33REF.1_1_104 | TACTGCCACAAATATCAATAGGAAC | | |
| | ATCACTTTTATTAGCCTGCAATGTT | | |
| | TTAAAATCCATGCAACCAAATC | | |
| | CTGTGTCCACCATATCACCATCC | | |
| 625339_25353839_333049_ | TAAAAGTGATGTTCCTATTGATATT | 120 | 805 |
| HPV33REF.1_1_105 | TGTGGCAGTACATGCAAATATCCAG | | |
| | ATTATTTAAAAATGACTAGTGAGCC | | |
| | TTATGGTGATAGTTTATTTTT | | |
| | CTTTCTTCGACGTGAACAAATGTT | | |
| 625339_25353839_333049_ | TACAGGTCATCGGGAACAGCCTCTC | 120 | 806 |
| HPV33REF.1_1_106 | CTAATGTACCAGCCCTATTAAAAAA | | |
| | GTGTCTTACAAACATTTGTTCACGT | | |
| | CGAAGAAAGAAAAATAAACT | | |
| | ATCACCATAAGGCTCACTAGTCATT | | |
| 625339_25353839_333049_ | TGTAAGACACTTTTTTAATAGGGCT | 120 | 807 |
| HPV33REF.1_1_107 | GGTACATTAGGAGAGGCTGTTCCCG | | |
| | ATGACCTGTACATTAAAGGTTCAGG | | |
| | AACTACTGCCTCTATTCAAA | | |
| | GCAGTGCTTTTTTTCCCACTCCTAG | | |
| 625339_25353839_333049_ | GCACGTTGTAGCCAATATGGCTTAT | 120 | 808 |
| HPV33REF.1_1_108 | TAAATAACTGAGATTCGGAAGTAAC | | |
| | CATTGATCCACTAGGAGTGGGAAAA | | |
| | AAAGCACTGCTTTGAATAGA | | |
| | GGCAGTAGTTCCTGAACCTTTAATG | | |
| 625339_25353839_333049_ | TGGATCAATGGTTACTTCCGAATCT | 120 | 809 |
| HPV33REF.1_1_109 | CAGTTATTTAATAAGCCATATTGGC | | |
| | TACAACGTGCACAAGGTCATAATAA | | |
| | TGGTATTTGTTGGGGCAATC | | |
| | AGGTATTTGTTACTGTGGTAGATAC | | |
| 625339_25353839_333049_ | TTTTTATATGTACTGTCACTAGTTA | 120 | 810 |
| HPV33REF.1_1_110 | CTTGTGTGCATAAAGTCATATTAGT | | |
| | ACTGCGAGTGGTATCTACCACAGTA | | |
| | ACAAATACCTGATTGCCCCAA | | |
| | CAAATACCATTATTATGACCTTGT | | |
| 625339_25353839_333049_ | CACTCGCAGTACTAATATGACTTTA | 120 | 811 |
| HPV33REF.1_1_111 | TGCACACAAGTAACTAGTGACAGTA | | |
| | CATATAAAAATGAAAATTTTAAAGA | | |
| | ATATATAAGACATGTTGAAGA | | |
| | ATATGATCTACAGTTTGTTTTTCA | | |
| 625339_25353839_333049_ | TCTGGATTCATAGCATGAATATATG | 120 | 812 |
| HPV33REF.1_1_112 | TCATAACTTCTGCAGTTAAGGTAAC | | |
| | TTTGCATAGTTGAAAAACAAACTGT | | |
| | AGATCATATTCTTCAACATGT | | |
| | CTTATATATTCTTTAAAATTTTCA | | |
| 625339_25353839_333049_ | ACTATGCAAAGTTACCTTAACTGCA | 120 | 813 |
| HPV33REF.1_1_113 | GAAGTTATGACATATATTCATGCTA | | |
| | TGAATCCAGATATTTTAGAAGATTG | | |
| | GCAATTTGGTTTAACACCTCC | | |
| | TCCATCTGCTAGTTTACAGGATAC | | |
| 625339_25353839_333049_ | TTTTCCTTTGGAGGTACTGTTTTTT | 120 | 814 |
| HPV33REF.1_1_114 | GACACGTAATAGCCTGAGAGGTAAC | | |
| | AAACCTATAGGTATCCTGTAAACTA | | |
| | GCAGATGGAGGAGGTGTTAA | | |
| | ACCAAATTGCCAATCTTCTAAAATA | | |
| 625339_25353839_333049_ | CTATAGGTTTGTTACCTCTCAGGCT | 120 | 815 |
| HPV33REF.1_1_115 | ATTACGTGTCAAAAAACAGTACCTC | | |
| | CAAAGGAAAAGGAAGACCCCTTAGG | | |
| | TAAATATACATTTTGGGAAG | | |
| | TGGATTTAAAGGAAAAATTTTCAGC | | |
| 625339_25353839_333049_ | TTTGCTTTAAGACCTGCCTGTAATA | 120 | 816 |
| HPV33REF.1_1_116 | AAAACTTGCGTCCCAAAGGAAACTG | | |
| | ATCTAAATCTGCTGAAAATTTTTCC | | |
| | TTTAAATCCACTTCCCAAAAT | | |
| | GTATATTTACCTAAGGGGTCTTCC | | |
| 625339_25353839_333049_ | AGATTTAGATCAGTTTCCTTTGGGA | 120 | 817 |
| HPV33REF.1_1_117 | CGCAAGTTTTTATTACAGGCAGGTC | | |
| | TTAAAGCAAAACCTAAACTTAAACG | | |
| | TGCAGCCCCCACATCCACCC | | |
| | GCACATCGTCTGCAAAACGCAAAAA | | |
| 625339_25353839_333049_ | GTACATAGACAGAACAAAACAACAA | 120 | 818 |
| HPV33REF.1_1_118 | CATAACACAATTACACAAAGTGTTA | | |
| | TTTTTTAACCTTTTTGCGTTTTGCA | | |
| | GACGATGTGCGGGTGGATGT | | |
| | GGGGGCTGCACGTTTAAGTTTAGGT | | |
| 625339_25353839_333049_ | GGTTAAAAAATAACACTTTGTGTAA | 120 | 819 |
| HPV33REF.1_1_119 | TTGTGTTATGTTGTTGTTTTGTTCT | | |
| | GTCTATGTACTTTGTGTTGTTGTGT | | |
| | TGTGTTGTTGTTTGTTTTTTGT | | |
| | GTATGTGTTACAATGTATGTTAT | | |
| 625339_25353839_333049_ | CAAGTACATAGAACATGCACACAAA | 120 | 820 |
| HPV33REF.1_1_120 | CAAGTACACATAAAACAAACACAGT | | |
| | AACATACAACATAACATACATTGTA | | |
| | ACACATACACAAAAAACAAAC | | |
| | AACAACACAACACAACAACACAAA | | |
| 625339_25353839_333049_ | GTTGTATGTTACtgtgtttgtttta | 120 | 821 |
| HPV33REF.1_1_121 | tgtgtacttgtttgtgtgcatgttc | | |
| | tatgtacttgtcagtttcctgtttg | | |
| | tgtatatgttAATAAAACATTGTGT | | |
| | GTATTTGTTAAACTATTTGT | | |
| 625339_25353839_333049_ | GGTAGGGCAAGCAATACAACTCCTT | 120 | 822 |
| HPV33REF.1_1_122 | ACTCATATAGGTACACCCATATACA | | |
| | TAACATACATACAAATAGTTTAACA | | |
| | AATACACACAATGTTTTATTA | | |
| | ACATATACACAAACAGGAAACTGA | | |
| 625339_25353839_333049_ | ATGTATGTTATGTATATGGGTGTAC | 120 | 823 |
| HPV33REF.1_1_123 | CTATATGAGTAAGGAGTTGTATTGC | | |
| | TTGCCCTACCCTGCATTGCAATGTA | | |
| | CCTACCTTTATTTCCCTATAT | | |
| | TTGTAGTACCTACATGTTTAGTAT | | |
| 625339_25353839_333049_ | AGGCATACAAAATGGAGGAAATTGT | 120 | 824 |
| HPV33REF.1_1_124 | ACAATATGGACACTAGTATGTCAAA | | |
| | AGGTAAAGCAATACTAAACATGTAG | | |
| | GTACTACAAATATAGGGAAA | | |
| | TAAAGGTAGGTACATTGCAATGCAG | | |
| 625339_25353839_333049_ | TGCTTTACCTTTTGACATACTAGTG | 120 | 825 |
| HPV33REF.1_1_125 | TCCATATTGTACAATTTCCTCCATT | | |
| | TTGTATGCCTAACCGTTTTCGGTTA | | |
| | CTTGGCATACATACCCTATGA | | |
| | CATTGGCAGAACAGTTAATCCTTT | | |
| 625339_25353839_333049_ | CAATGTCATAGGGTATGTATGCCAA | 120 | 826 |
| HPV33REF.1_1_126 | TGCAGCAAGTACAGACAAACACAGT | | |
| | GCAGGAAAGAAAAGGATTAACTGTT | | |
| | CTGCCAATGTCATAGGGTA | | |
| | TGTATGCCAAGTAACCGAAAACGGT | | |
| | T | | |
| 625339_25353839_333049_ | TCTTTCCTGCACTGTGTTTGTCTGT | 120 | 827 |
| HPV33REF.1_1_127 | ACTTGCTGCATTGGCATACATACCC | | |
| | TATGACATTGGCAGAACAGTTAATC | | |
| | CTTTTCTTTCCTGCACTGTGT | | |
| | TTGTCTGTACTTGCTGCATTGACT | | |
| 625339_25353839_333049_ | AAGCATGTGTAAACTATTAGTACAA | 120 | 828 |
| HPV33REF.1_1_128 | TTAAGTATTTTGCAATTGCACTGCA | | |
| | TGTATATATGAGTCAATGCAGCAAG | | |
| | TACAGACAAACACAGTGCAG | | |
| | GAAAGAAAAGGATTAACTGTTCTGC | | |
| 625339_25353839_333049_ | CATATATACATGCAGTGCAATTGCA | 120 | 829 |
| HPV33REF.1_1_129 | AAATACTTAATTGTACTAATAGTTT | | |
| | ACACATGCTTTTAGGCACATATTTT | | |
| | TACTTTACTTTCAAACCTTAAG | | |
| | TGCAGTTTTGGCTTACACAATTG | | |
| 625339_25353839_333049_ | CACACCTGGTAATAAACAGGTAGTG | 120 | 830 |
| HPV33REF.1_1_130 | ACTCACTTTTACAAGGCATAGTTTG | | |
| | GCATACAAAGCAATTGTGTAAGCCA | | |
| | AAACTGCACTTAAGGTTTGA | | |
| | AAGTAAAGTAAAAATATGTGCCTAA | | |
| 625339_25353839_333049_ | CTTTGTATGCCAAACTATGCCTTGT | 109 | 831 |
| HPV33REF.1_1_131 | AAAAGTGAGTCACTACCTGTTTATT | | |
| | ACCAGGTGTGGACTAACCGTTTTAG | | |
| | GTCATATTGGTCATTTATAAT | | |
| | CTTTTATATAATA | | |

| HPV35_DoubleStrand_ | | | SEQ |
|---|---|---|---|
| SequenceName | Sequence | Length | ID#: |
| 625339_25353836_396997_ | CCCTATAAAAAAAACAGGGAGTGAC | 120 | 832 |
| HPV35REF.1_1_1 | CGAAAACGGTCGTACCGAAAACGGT | | |
| | TGCCATAAAAGCAGAAGTGCACAAA | | |
| | AAAGCAGAAGT | | |
| | GGACAGACATTGTAAGGTGCGGTAT | | |
| | GTTTCAGGA | | |
| 625339_25353836_396997_ | TGGATGCTTTCTTCTACCTCGTTGC | 120 | 833 |
| HPV35REF.1_1_2 | ACAAATCATGCAGTTTGTAAGGTCG | | |
| | TTCAGCTGGGTCCTGAAACATACCG | | |
| | CACCTTACAATG | | |
| | TCTGTCCACTTCTGCTTTTTTGTGC | | |
| | ACTTCTGC | | |
| 625339_25353836_396997_ | CCCAGCTGAACGACCTTACAAACTG | 120 | 834 |
| HPV35REF.1_1_3 | CATGATTTGTGCAACGAGGTAGAAG | | |
| | AAAGCATCCATGAAATTTGTTTGAA | | |
| | TTGTGTATACTG | | |
| | CAAACAAGAATTACAGCGGAGTGAG | | |
| | GTATATGA | | |
| 625339_25353836_396997_ | ATGCATACTCCATATGGCTGGCCTT | 120 | 835 |
| HPV35REF.1_1_4 | CTCTATATACTATACACAAATCATA | | |
| | GCATGCAAAGTCATATACCTCACTC | | |
| | CGCTGTAATTCTT | | |
| | GTTTGCAGTATACACAATTCAAACA | | |
| | AATTTCA | | |
| 625339_25353836_396997_ | CTTTGCATGCTATGATTTGTGTATA | 120 | 836 |
| HPV35REF.1_1_5 | GTATATAGAGAAGGCCAGCCATATG | | |
| | GAGTATGCATGAAATGTTTAAAATT | | |
| | TTATTCAAAAATA | | |
| | AGTGAATATAGATGGTATAGATATA | | |
| | GTGTGTA | | |
| 625339_25353836_396997_ | ATACACCTAATTAATAAATGACATA | 120 | 837 |
| HPV35REF.1_1_6 | ACTGTTTGTTGCATTGTTTTTCTAA | | |
| | CGTTTCTCCATACACACTATATCTA | | |
| | TACCATCTATATTC | | |
| | ACTTATTTTTGAATAAAATTTTAAA | | |
| | CATTTC | | |
| 625339_25353836_396997_ | TGGAGAAACGTTAGAAAAACAATGC | 120 | 838 |
| HPV35REF.1_1_7 | AACAAACAGTTATGTCATTTATTAA | | |
| | TTAGGTGTATTACATGTCAAAAACC | | |
| | GCTGTGTCCAGTT | | |
| | GAAAAGCAAAGACATTTAGAAGAAA | | |
| | AAAAACG | | |
| 625339_25353836_396997_ | CGTGTTGGTTTCCAACAGGACATAC | 120 | 839 |
| HPV35REF.1_1_8 | ACCGACCTGTCCACCGTCCACCGAT | | |
| | GTTATGGAATCGTTTTTTTTCTTCT | | |
| | AAATGTCTTTGC | | |
| | TTTTCAACTGGACACAGCGGTTTTT | | |
| | GACATGTA | | |
| 625339_25353836_396997_ | ATTCCATAACATCGGTGGACGGTGG | 120 | 840 |
| HPV35REF.1_1_9 | ACAGGTCGGTGTATGTCCTGTTGGA | | |
| | AACCAACACGTAGAGAAACCGAGGT | | |
| | GTAATCATGC | | |
| | ATGGAGAAATAACTACATTGCAAGA | | |
| | CTATGTTTTA | | |
| 625339_25353836_396997_ | CTCTGAGCTGTCACACAATTGCTCA | 120 | 841 |
| HPV35REF.1_110 | TAACAGTATAGGTCAGTTGCCTCGG | | |
| | GTTCCAAATCTAAAACATAGTCTTG | | |
| | CAATGTAGTTAT | | |
| | TTCTCCATGCATGATTACACCTCGG | | |
| | TTTCTCTA | | |
| 625339_25353836_396997_ | GATTTGGAACCCGAGGCAACTGACC | 120 | 842 |
| HPV35REF.1_1_11 | TATACTGTTATGAGCAATTGTGTGA | | |
| | CAGCTCAGAGGAGGAGGAAGATACT | | |
| | ATTGACGGTCC | | |
| | AGCTGGACAAGCAAAACCAGACACC | | |
| | TCCAATTAT | | |
| 625339_25353836_396997_ | TGTGCTCTGTACACACAGACGTAGT | 120 | 843 |
| HPV35REF.1_1_12 | GTCGCCTCACATTTACAACAGGACG | | |
| | TTACAATATTATAATTGGAGGTGTC | | |
| | TGGTTTTGCTTG | | |
| | TCCAGCTGGACCGTCAATAGTATCT | | |
| | TCCTCCTC | | |
| 625339_25353836_396997_ | AATATTGTAACGTCCTGTTGTAAAT | 120 | 844 |
| HPV35REF.1_1_13 | GTGAGGCGACACTACGTCTGTGTGT | | |
| | ACAGAGCACACACATTGACATACGT | | |
| | AAATTGGAAGAT | | |
| | TTATTAATGGGCACATTTGGAATAG | | |
| | TGTGCCCC | | |
| 625339_25353836_396997_ | CTCCCCTTCATCTGTACCTGCAGGA | 120 | 845 |
| HPV35REF.1_1_14 | TCAGCCATTGTAGATTATGCTCTCT | | |
| | GTGAACAGCCGGGGCACACTATTCC | | |
| | AAATGTGCCCA | | |
| | TTAATAAATCTTCCAATTTACGTAT | | |
| | GTCAATGTG | | |
| 625339_25353836_396997_ | GGCTGTTCACAGAGAGCATAATCTA | 120 | 846 |
| HPV35REF.1_1_15 | CAATGGCTGATCCTGCAGGTACAGA | | |
| | TGAAGGGGAGGGGACGGGATGTAAT | | |
| | GGATGGTTTT | | |
| | TTGTAGAAGCAGTAGTTAGTAGACG | | |
| | TACGGGGGAT | | |
| 625339_25353836_396997_ | AAAGTCCACCATATCCTCCCCCCTG | 120 | 847 |
| HPV35REF.1_1_16 | TCACAGTCATCTTCATTTTCGTCCT | | |
| | CTGACACTGGATCCCCCGTACGTCT | | |
| | ACTAACTACTGC | | |
| | TTCTACAAAAAACCATCCATTACAT | | |
| | CCCGTCCC | | |
| 625339_25353836_396997_ | CCAGTGTCAGAGGACGAAAATGAAG | 120 | 848 |
| HPV35REF.1_1_17 | ATGACTGTGACAGGGGGGAGGATAT | | |
| | GGTGGACTTTATAAATGATACAGAT | | |
| | ATATTAAACAT | | |
| | ACAGGCAGAAACAGAGACAGCACAA | | |
| | GCATTATTT | | |
| 625339_25353836_396997_ | AGCATACTTTCGTTTTAGGACCTGT | 120 | 849 |
| HPV35REF.1_1_18 | ACAGCCTCTTTGTGTGTTTGCTCCT | | |
| | CCTGTGCATGAAATAATGCTTGTGC | | |
| | TGTCTCTGTTTC | | |
| | TGCCTGTATGTTTAATATATCTGTA | | |
| | TCATTTAT | | |
| 625339_25353836_396997_ | CATGCACAGGAGGAGCAAACACACA | 120 | 850 |
| HPV35REF.1_1_19 | AAGAGGCTGTACAGGTCCTAAAACG | | |
| | AAAGTATGCTAGTAGTCCACTTAGC | | |
| | AGCGTGAGCTT | | |
| | ATGTGTTAATAATAACATAAGTCCA | | |
| | CGTTTAAAA | | |
| 625339_25353836_396997_ | GTCTGGTAGTTCAAATAATCGTCGC | 120 | 851 |
| HPV35REF.1_1_20 | TTTGCTGCTGTATTTTTATTTTCAA | | |
| | TGCAAATAGCTTTTAAACGTGGACT | | |
| | TATGTTATTATTAA | | |
| | CACATAAGCTCACGCTGCTAAGTGG | | |
| | ACTACT | | |
| 625339_25353836_396997_ | GCTATTTGCATTGAAAATAAAAATA | 120 | 852 |
| HPV35REF.1_1_21 | CAGCAGCAAAGCGACGATTATTTGA | | |
| | ACTACCAGACAGCGGTTATGGCAAT | | |
| | TCTGAAGTGGAA | | |
| | ATACAGCAGATACAACAGGTAGAGG | | |
| | GGCATGAT | | |
| 625339_25353836_396997_ | TCTTTCATCGCTACTAGAGGTTATA | 120 | 853 |
| HPV35REF.1_1_22 | CTATCCCCACTGCCCATACTACATT | | |
| | GTTCAACTGTATCATGCCCCTCTAC | | |
| | CTGTTGTATCTGC | | |
| | TGTATTTCCACTTCAGAATTGCCAT | | |
| | AACCGCT | | |
| 625339_25353836_396997_ | ACAGTTGAACAATGTAGTATGGGCA | 120 | 854 |
| HPV35REF.1_1_23 | GTGGGGATAGTATAACCTCTAGTAG | | |
| | CGATGAAAGACATGATGAGACTCCA | | |
| | ACGCGAGACAT | | |
| | AATACAAATACTAAAATGTAGTAAT | | |
| | GCAAACGCA | | |
| 625339_25353836_396997_ | TGGTCTAATAAGTTCTGTAAAACTA | 120 | 855 |
| HPV35REF.1_1_24 | ATACCAAATAGTTCTTTAAATTTAG | | |
| | CCAACATAGCTGCGTTTGCATTACT | | |
| | ACATTTTAGTATTT | | |
| | GTATTATGTCTCGCGTTGGAGTCTC | | |
| | ATCATG | | |
| 625339_25353836_396997_ | GCTATGTTGGCTAAATTTAAAGAAC | 120 | 856 |
| HPV35REF.1_1_25 | TATTTGGTATTAGTTTTACAGAACT | | |
| | TATTAGACCATTTAAGAGTGATAAA | | |
| | TCCACATGTACAG | | |
| | ATTGGTGTGTGGCCGCATTTGGAAT | | |
| | AGCCCCA | | |
| 625339_25353836_396997_ | TTGTATATGTATATATAAACAATAT | 120 | 857 |
| HPV35REF.1_1_26 | GGTTTAATTAATGTTTTTAAACTTT | | |
| | CCGCCACACTTGGGGCTATTCCAAA | | |
| | TGCGGCCACACAC | | |
| | CAATCTGTACATGTGGATTTATCAC | | |
| | TCTTAAA | | |
| 625339_25353836_396997_ | AGTGTGGCGGAAAGTTTAAAAACAT | 120 | 858 |
| HPV35REF.1_1_27 | TAATTAAACCATATTGTTTATATAT | | |
| | ACATATACAATGTTTATCGTGTTCA | | |
| | TGGGGTATGGTAA | | |
| | TTCTAGCATTATTACGATTTAAATG | | |
| | TGCAAAA | | |
| 625339_25353836_396997_ | CATACTTGCAGCTGAAATACATAGC | 120 | 859 |
| HPV35REF.1_1_28 | AATTTTGATAATAGTTTTTCAATTG | | |
| | TTGTTCTGTTTTTTGCACATTTAAA | | |
| | TCGTAATAATGCTA | | |
| | GAATTACCATACCCCATGAACACGA | | |
| | TAAACA | | |
| 625339_25353836_396997_ | AACAGAACAACAATTGAAAAACTAT | 120 | 860 |
| HPV35REF.1_1_29 | TATCAAAATTGCTATGTATTTCAGC | | |
| | TGCAAGTATGCTAATACAACCACCA | | |
| | AAATTACGTAGTA | | |
| | CCCCAGCTGCGTTATATTGGTTTAA | | |
| | AACAGCA | | |
| 625339_25353836_396997_ | TACTGTTTGTCTTTGAATCCATTCT | 120 | 861 |
| HPV35REF.1_1_30 | GGTGTTTCTCCATCAACCTCACTAA | | |
| | TATTTGACATTGCTGTTTTAAACCA | | |
| | ATATAACGCAGCT | | |
| | GGGGTACTACGTAATTTTGGTGGTT | | |
| | GTATTAG | | |
| 625339_25353836_396997_ | ATGTCAAATATTAGTGAGGTTGATG | 120 | 862 |
| HPV35REF.1_1_31 | GAGAAACACCAGAATGGATTCAAAG | | |
| | ACAAACAGTATTACAGCATAGTTTT | | |
| | AATGATGCAATA | | |
| | TTTGACCTATCTGAAATGGTACAAT | | |
| | GGGCATAT | | |
| 625339_25353836_396997_ | AGTTTCTGCCAATTGTGCATATTTA | 120 | 863 |
| HPV35REF.1_1_32 | TATGCTATATCACTATCATCTATAA | | |
| | AATCATTGTCATATGCCCATTGTAC | | |
| | CATTTCAGATAGGT | | |
| | CAAATATTGCATCATTAAAACTATG | | |
| | CTGTAA | | |
| 625339_25353836_396997_ | GACAATGATTTTATAGATGATAGTG | 120 | 864 |
| HPV35REF.1_1_33 | ATATAGCATATAAATATGCACAATT | | |
| | GGCAGAAACTAATAGTAATGCATGT | | |
| | GCTTllTTAAAAA | | |
| | GTAATTCGCAAGCTAAAATTGTAAA | | |
| | AGATTGT | | |
| 625339_25353836_396997_ | CCACTGTGACATTGTCATTTCTCTT | 120 | 865 |
| HPV35REF.1_1_34 | TTTTCAGCTCGTTTATAATGTCTAC | | |
| | ACATTGTTGCACAATCTTTTACAAT | | |
| | TTTAGCTTGCGAAT | | |
| | TACTTTTTAAAAAAGCACATGCATT | | |
| | ACTATT | | |
| 625339_25353836_396997_ | GCAACAATGTGTAGACATTATAAAC | 120 | 866 |
| HPV35REF.1_1_35 | GAGCTGAAAAAAGAGAAATGACAAT | | |
| | GTCACAGTGGATTAAAAGGCGATGT | | |
| | GAAAAGGTGGA | | |
| | CGATGACGGTGACTGGAGGGACATA | | |
| | GTACGATTT | | |
| 625339_25353836_396997_ | ATGTAAAAAATTTTTTAGTGCAGAT | 120 | 867 |
| HPV35REF.1_1_36 | AAAAATGCCACAAAATcTACTTGTT | | |
| | GATATCTTAAAAATCGTACTATGTc | | |
| | eCTCCAGTCACCG | | |
| | TCATCGTCCACCTTTTCACATCGCC | | |
| | TTTTAAT | | |
| 625339_25353836_396997_ | TTAAGATATCAACAAGTAGATTTTG | 120 | 868 |
| HPV35REF.1_1_37 | TGGCATTTTTATcTGCACTAAAAAA | | |
| | TTTTTTACATGGTGTGCCTAAAAAA | | |
| | AATTGCATACTTAT | | |
| | ATATGGAGCACCAAACACAGGTAAA | | |
| | TCATTA | | |
| 625339_25353836_396997_ | TTTAGAATTTACATAGGATATAATA | 120 | 869 |
| HPV35REF.1_1_38 | GCTCCTTGTAAGAAATGCATTAGAC | | |
| | TCATTCCAAATAATGATTTACCTGT | | |
| | GTTTGGTGCTCCA | | |
| | TATATAAGTATGCAATTTTTTTTAG | | |
| | GCACACC | | |
| 625339_25353836_396997_ | TTTGGAATGAGTCTAATGCATTTCT | 120 | 870 |
| HPV35REF.1_1_39 | TACAAGGAGCTATTATATCCTATGT | | |
| | AAATTCTAAAAGCCATTTTTGGTTG | | |
| | CAGCCATTATATG | | |
| | ATGCCAAAATAGCTATGTTAGATGA | | |
| | TGCTACA | | |
| 625339_25353836_396997_ | AATAGGATTTCCATCTAGTGCATTT | 120 | 871 |
| HPV35REF.1_1_40 | CTTAAATATTGGTCTATATATGCCC | | |
| | AACATGGCGATGTAGCATCATCTAA | | |
| | CATAGCTATTTTG | | |
| | GCATCATATAATGGCTGCAACCAAA | | |
| | AATGGCT | | |
| 625339_25353836_396997_ | TCGCCATGTTGGGCATATATAGACC | 120 | 872 |
| HPV35REF.1_1_41 | AATATTTAAGAAATGCACTAGATGG | | |
| | AAATCCTATTTCATTAGATGTAAAG | | |
| | CATAAAGCATTAG | | |
| | TGCAATTAAAATGCCCACCTTTACT | | |
| | TATTACA | | |
| 625339_25353836_396997_ | GACCACTACCCTGCTATGTAAGTAT | 120 | 873 |
| HPV35REF.1_1_42 | GGCCACCTGTCATCTTTGCCTGCAT | | |
| | TTATATTTGATGTAATAAGTAAAGG | | |
| | TGGGCATTTTAA | | |
| | TTGCACTAATGCTTTATGCTTTACA | | |
| | TCTAATGA | | |
| 625339_25353836_396997_ | TCAAATATAAATGCAGGCAAAGATG | 120 | 874 |
| HPV35REF.1_1_43 | ACAGGTGGCCATACTTACATAGCAG | | |
| | GGTAGTGGTCTTTACATTTCACAAT | | |
| | GAATTCCCATTT | | |
| | GATAAAAATGGAAACCCAGTGTATG | | |
| | GGCTTAAT | | |
| 625339_25353836_396997_ | TTCCTCGTGCAAATTTAATCTGCAC | 120 | 875 |
| HPV35REF.1_1_44 | CACGTCCTTGAGAAAAAGGATTTCC | | |
| | AGTTTTTATCATTAAGCCCATACAC | | |
| | TGGGTTTCCATTT | | |
| | TTATCAAATGGGAATTCATTGTGAA | | |
| | ATGTAAA | | |
| 625339_25353836_396997_ | GATAAAAACTGGAAATCCTTTTTCT | 120 | 876 |
| HPV35REF.1_1_45 | CAAGGACGTGGTGCAGATTAAATTT | | |
| | GCACGAGGAAGAGGACAAAGAAAAT | | |
| | GATGGAGACGC | | |
| | TTTCCCAGCGTTTAAGTGTGTGTCA | | |
| | GGACAAAAT | | |
| 625339_25353836_396997_ | AGTTTCCAATACTGTATGTGATCAG | 120 | 877 |
| HPV35REF.1_1_46 | ACAAACATGTGCTATCAGTCTCGTA | | |
| | ATGTTCTAGTATTTTGTCCTGACAC | | |
| | ACACTTAAACGCT | | |
| | GGGAAAGCGTCTCCATCATTTTCTT | | |
| | TGTCCTC | | |
| 625339_25353836_396997_ | ACTAGAACATTACGAGACTGATAGC | 120 | 878 |
| HPV35REF.1_1_47 | ACATGTTTGTCTGATCACATACAGT | | |
| | ATTGGAAACTGATTCGTCTTGAATG | | |
| | TGCAGTATTTTAT | | |
| | AAAGCAAGAGAAATGGGAATTAAAA | | |
| | CTCTTAA | | |
| 625339_25353836_396997_ | AGTTCAATTGCTTGCATTGCTTTGG | 120 | 879 |
| HPV35REF.1_1_48 | CTTTTGAAATGGCCTGCGTTGGAAC | | |
| | CACTTGGTGGTTAAGAGTTTTAATT | | |
| | CCCATTTCTCTT | | |
| | GCTTTATAAAATACTGCACATTCAA | | |
| | GACGAATC | | |
| 625339_25353836_396997_ | CCACCAAGTGGTTCCAACGCAGGCC | 120 | 880 |
| HPV35REF.1_1_49 | ATTTCAAAAGCCAAAGCAATGCAAG | | |
| | CAATTGAACTGCAATTAATGTTAGA | | |
| | GACATTAAATAC | | |
| | AACTGAGTATAGCACAGAAACATGG | | |
| | ACACTGCA | | |
| 625339_25353836_396997_ | ACCCCATGTTTTTTAAAACATCCTT | 120 | 881 |
| HPV35REF.1_1_50 | GTGGAACTGTTGTATATAATTCAAT | | |
| | ACTTGTTTCTTGCAGTGTCCATGTT | | |
| | TCTGTGCTATACT | | |
| | CAGTTGTATTTAATGTCTCTAACAT | | |
| | TAATTGC | | |
| 625339_25353836_396997_ | AGAAACAAGTATTGAATTATATACA | 120 | 882 |
| HPV35REF.1_1_51 | ACAGTTCCACAAGGATGTTTTAAAA | | |
| | AACATGGGGTTACAGTGGAAGTACA | | |
| | ATTTGATGGTGA | | |
| | TAAACAAAATACTATGCATTATACT | | |
| | AATTGGAC | | |
| 625339_25353836_396997_ | TTATAATTTACCAGTCCCTTTACAA | 120 | 883 |
| HPV35REF.1_1_52 | CAGTACATATACTGTCCTCTAATAT | | |
| | ATATATATGTGTCCAATTAGTATAA | | |
| | TGCATAGTATTTTG | | |
| | TTTATCACCATCAAATTGTACTTCC | | |
| | ACTGTA | | |
| 625339_25353836_396997_ | ACATATATATATATTAGAGGACAGT | 120 | 884 |
| HPV35REF.1_1_53 | ATATGTACTGTTGTAAAGGGACTGG | | |
| | TAAATTATAAAGGTATTTATTATGT | | |
| | GCATCAGGGTGTA | | |
| | GAAACATATTATGTTACTTTTAGGG | | |
| | AAGAGGC | | |
| 625339_25353836_396997_ | CAAACAATTACCTGACCACCCACAT | 120 | 885 |
| HPV35REF.1_1_54 | GCACTTCCCATATATTTTTTTTTCC | | |
| | ATACTTTTTAGCCTCTTCCCTAAAA | | |
| | GTAACATAATATGT | | |
| | TTCTACACCCTGATGCACATAATAA | | |
| | ATACCT | | |
| 625339_25353836_396997_ | TAAAAAGTATGGAAAAAAAAATATA | 120 | 886 |
| HPV35REF.1_1_55 | TGGGAAGTGCATGTGGGTGGTCAGG | | |
| | TAATTGTTTGTCCTGAATCTGTATT | | |
| | TAGCAGCACAGA | | |
| | ACTATCCACTGCTGAAATTGCTACA | | |
| | CAGCTACA | | |
| 625339_25353836_396997_ | TGGGTTTCTGTGGTGCCCACGGAGC | 120 | 887 |
| HPV35REF.1_1_56 | AGGCTTTGGTATGGGTCTCGGTGGT | | |
| | GTTGTAGGCGTGTAGCTGTGTAGCA | | |
| | ATTTCAGCAG | | |
| | TGGATAGTTCTGTGCTGCTAAATAC | | |
| | AGATTCAGGA | | |
| 625339_25353836_396997_ | CGCCTACAACACCACCGAGACCCAT | 120 | 888 |
| HPV35REF.1_1_57 | ACCAAAGCCTGCTCCGTGGGCACCA | | |
| | CAGAAACCCAGAAGACAAATCACAA | | |
| | ACGACTTCGAG | | |
| | GGGGTACCGAGCTCCCCTACAACCC | | |
| | CACCAAGCG | | |
| 625339_25353836_396997_ | GTGCAGTCAGATGTAGAGTAGACCC | 120 | 889 |
| HPV35REF.1_1_58 | CTCTGTCAACACTGTCCACGGCACT | | |
| | GAGTCGCACTCGCTTGGTGGGGTTG | | |
| | TAGGGGAGCT | | |
| | CGGTACCCCCTCGAAGTCGTTTGTG | | |
| | ATTTGTCTTC | | |
| 625339_25353836_396997_ | AGTGCGACTCAGTGCCGTGGACAGT | 120 | 890 |
| HPV35REF.1_1_59 | GTTGACAGAGGGGTCTACTCTACAT | | |
| | CTGACTGCACAAACAAAGACCGGTG | | |
| | TGGTAGTTGT | | |
| | AGTACAACTACACCTATAGTACATT | | |
| | TAAAAGGTGA | | |
| 625339_25353836_396997_ | TCTTGATACAATGCTTTATATTTAC | 120 | 891 |
| HPV35REF.1_1_60 | CCAATCTATATCTTAAACACTTTAA | | |
| | TGTATTTGCATCACCTTTTAAATGT | | |
| | ACTATAGGTGTAGT | | |
| | TGTACTACAACTACCACACCGGTCT | | |
| | TTGTTT | | |
| 625339_25353836_396997_ | TGCAAATACATTAAAGTGTTTAAGA | 120 | 892 |
| HPV35REF.1_1_61 | TATAGATTGGGTAAATATAAAGCAT | | |
| | TGTATCAAGATGCTTCATCTACATG | | |
| | GAGATGGACATGT | | |
| | ACAAACGATAAAAAACAAATAGCAA | | |
| | TTGTAAC | | |
| 625339_25353836_396997_ | TTAGGTATTTTTACTGTAGTTAAAA | 120 | 893 |
| HPV35REF.1_1_62 | ATTTATCCCTTTGATATTCTGTTGT | | |
| | GTAAGTTAATGTTACAATTGCTATT | | |
| | TGTTTTTTATCGTTT | | |
| | GTACATGTCCATCTCCATGTAGATG | | |
| | AAGCA | | |
| 625339_25353836_396997_ | ATTAACTTACACAACAGAATATCAA | 120 | 894 |
| HPV35REF.1_1_63 | AGGGATAAATTTTTAACTACAGTAA | | |
| | AAATACCTAACACAGTTACAGTGTC | | |
| | TAAAGGATATATGT | | |
| | CTATATGATAGACCTTACAGCTTCC | | |
| | AGTACT | | |
| 625339_25353836_396997_ | ACGTACAAGCAGACACAAGCACAAA | 120 | 895 |
| HPV35REF.1_1_64 | AGCACACAAAAGCACAACAAAAAGC | | |
| | ACAGCAACACAGTACTGGAAGCTGT | | |
| | AAGGTCTATCA | | |
| | TATAGACATATATCCTTTAGACACT | | |
| | GTAACTGTG | | |
| 625339_25353836_396997_ | GTGTTGCTGTGCTTTTTGTTGTGCT | 120 | 896 |
| HPV35REF.1_1_65 | TTTGTGTGCTTTTGTGCTTGTGTCT | | |
| | GCTTGTACGTTCGCTATTGCTATCT | | |
| | GTGTCATTATACT | | |
| | CAGCATTAATATTACTGGTTTTAAT | | |
| | ACTGTGG | | |
| 625339_25353836_396997_ | AGGTATATACAAAAAGCAAAGAAAA | 120 | 897 |
| HPV35REF.1_1_66 | CAACAAAAGCAACGTAGTGGTGTTG | | |
| | CTACAGTAACCCACAGTATTAAAAC | | |
| | CAGTAATATTAAT | | |
| | GCTGAGTATAATGACACAGATAGCA | | |
| | ATAGCGA | | |
| 625339_25353836_396997_ | GTTACTGTAGCAACACCACTACGTT | 120 | 898 |
| HPV35REF.1_1_67 | GCTTTTGTTGTTTTCTTTGCTTTTT | | |
| | GTATATACCTATGGGAATGATTAAC | | |
| | GCTCATGCACAAT | | |
| | ATTTGGCAGTACAGTAATTGTATAC | | |
| | AAACATT | | |
| 625339_25353836_396997_ | TATACAATGAACAACAAAAAATAAA | 120 | 899 |
| HPV35REF.1_1_68 | ACCACCATACACACATGTTACACAG | | |
| | TACCAAACACAATGTTTGTATACAA | | |
| | TTACTGTACTGCC | | |
| | AAATATTGTGCATGAGCGTTAATCA | | |
| | TTCCCAT | | |
| 625339_25353836_396997_ | GTGTTTGGTACTGTGTAACATGTGT | 120 | 900 |
| HPV35REF.1_1_69 | GTATGGTGGTTTTATTTTTTGTTGT | | |
| | TCATTGTATATTTTGTTTTTTTACT | | |
| | GTTTTTAAACATTT | | |
| | TTATTTCTGTGTTTTTAATAAATTG | | |
| | ATCACA | | |
| 625339_25353836_396997_ | TGTGTTGCAGATGCACGTTTAACAC | 120 | 901 |
| HPV35REF.1_1_70 | GTTTTGTAGACCTTTTGTGTCGCAT | | |
| | GGTTATACCATGTGATCAATTTATT | | |
| | AAAAACACAGAAA | | |
| | TAAAAATGTTTAAAAACAGTAAAAA | | |
| | AACAAAA | | |
| 625339_25353836_396997_ | TGGTATAACCATGCGACACAAAAGG | 120 | 902 |
| HPV35REF.1_1_71 | TCTACAAAACGTGTTAAACGTGCAT | | |
| | CTGCAACACAACTATATCGTACTTG | | |
| | CAAAGCTGCAGG | | |
| | AACTTGTCCACCAGATGTTATACCT | | |
| | AAGGTTGA | | |
| 625339_25353836_396997_ | CCCCCAAAAAACACAGCCATGCTGC | 120 | 903 |
| HPV35REF.1_1_72 | CATATTTTAAAATTTGATCAGCAAC | | |
| | AGTATTACCCTCAACCTTAGGTATA | | |
| | ACATCTGGTGGA | | |
| | CAAGTTCCTGCAGCTTTGCAAGTAC | | |
| | GATATAGT | | |
| 625339_25353836_396997_ | GGGTAATACTGTTGCTGATCAAATT | 120 | 904 |
| HPV35REF.1_1_73 | TTAAAATATGGCAGCATGGCTGTGT | | |
| | TTTTTGGGGGGTTAGGAATTGGTTC | | |
| | TGGATCTGGCA | | |
| | CAGGTGGAAGATCTGGATATGTTCC | | |
| | ACTGGGTAC | | |
| 625339_25353836_396997_ | ATACTTTCCACAGTTACAGGGGGTC | 120 | 905 |
| HPV35REF.1_1_74 | GTATAGGAATGTTTGTGGCAGCCGT | | |
| | TGGAGGTGTTGTACCCAGTGGAACA | | |
| | TATCCAGATCT | | |
| | TCCACCTGTGCCAGATCCAGAACCA | | |
| | ATTCCTAAC | | |
| 625339_25353836_396997_ | AACACCTCCAACGGCTGCCACAAAC | 120 | 906 |
| HPV35REF.1_1_75 | ATTCCTATACGACCCCCTGTAACTG | | |
| | TGGAAAGTATACCATTAGACACAAT | | |
| | TGGCCCTTTAGA | | |
| | TTCTTCTATAGTGTCATTAGTAGAG | | |
| | GAAACTAG | | |
| 625339_25353836_396997_ | GTAAAACCTGTTGTAGGTGGGACCC | 120 | 907 |
| HPV35REF.1_1_76 | TTGGTGTAACAACAGGGGCACCAGA | | |
| | CTCAATAAAACTAGTTTCCTCTACT | | |
| | AATGACACTATA | | |
| | GAAGAATCTAAAGGGCCAATTGTGT | | |
| | CTAATGGT | | |
| 625339_25353836_396997_ | TTTTATTGAGTCTGGTGCCCCTGTT | 120 | 908 |
| HPV35REF.1_1_77 | GTTACACCAAGGGTCCCACCTACAA | | |
| | CAGGTTTTACAATAACCACATCTAC | | |
| | AGATACCACACC | | |
| | TGCTATTTTAGATGTGACATCCATA | | |
| | AGTACACA | | |
| 625339_25353836_396997_ | GAAGTTTCTGCAGGCGTGGGTGGGT | 120 | 909 |
| HPV35REF.1_1_78 | GTAAAACAGAAGGATCAGTGAAAGT | | |
| | AGGATTATCATGTGTACTTATGGAT | | |
| | GTCACATCTAA | | |
| | AATAGCAGGTGTGGTATCTGTAGAT | | |
| | GTGGTTATT | | |
| 625339_25353836_396997_ | TGATAATCCTACTTTCACTGATCCT | 120 | 910 |
| HPV35REF.1_1_79 | TCTGTTTTACACCCACCCACGCCTG | | |
| | CAGAAACTTCAGGTCATTTTGTACT | | |
| | TTCATCATCTTCT | | |
| | ATTAGTACACATAATTATGAAGAAA | | |
| | TCCCTAT | | |
| 625339_25353836_396997_ | CCTGGAATAGGCGTGCTATTAGTTA | 120 | 911 |
| HPV35REF.1_1_80 | TATTATTGCTGTCTGTGGAAACAAT | | |
| | AAAAGTATCCATAGGGATTTCTTCA | | |
| | TAATTATGTGTAC | | |
| | TAATAGAAGATGATGAAAGTACAAA | | |
| | ATGACCT | | |
| 625339_25353836_396997_ | GGATACTTTTATTGTTTCCACAGAC | 120 | 912 |
| HPV35REF.1_1_81 | AGCAATAATATAACTAATAGCACGC | | |
| | CTATTCCAGGGTCTCGCCCTACGAC | | |
| | ACGCCTAGGATT | | |
| | ATATAGTAAAGGTACCCAGCAGGTT | | |
| | AAGGTTGT | | |
| 625339_25353836_396997_ | TCATATGCAGGATTATCATATGTAA | 120 | 913 |
| HPV35REF.1_1_82 | TAAGTTTTGCAGGAGAAGTCATAAA | | |
| | GGCAGGGTCAACAACCTTAACCTGC | | |
| | TGGGTACCTTTA | | |
| | CTATATAATCCTAGGCGTGTCGTAG | | |
| | GGCGAGAC | | |
| 625339_25353836_396997_ | TGACCCTGCCTTTATGACTTCTCCT | 120 | 914 |
| HPV35REF.1_1_83 | GCAAAACTTATTACATATGATAATC | | |
| | CTGCATATGAAGGCCTTAACCCTGA | | |
| | TACAACCTTACAA | | |
| | TTTGAGCATGAGGATATTAGCTTAG | | |
| | CTCCGGA | | |
| 625339_25353836_396997_ | GTGCCTTTCCTAGATGTTAGTGCAG | 120 | 915 |
| HPV35REF.1_1_84 | GCCTATGTAAAGCTATAATGTCCAT | | |
| | AAAGTCAGGATCCGGAGCTAAGCTA | | |
| | ATATCCTCATGC | | |
| | TCAAATTGTAAGGTTGTATCAGGGT | | |
| | TAAGGCCT | | |
| 625339_25353836_396997_ | TCCTGACTTTATGGACATTATAGCT | 120 | 916 |
| HPV35REF.1_1_85 | TTACATAGGCCTGCACTAACATCTA | | |
| | GGAAAGGCACTATTAGATATAGTAG | | |
| | AGTAGGTAATAA | | |
| | ACGTACTATGCATACACGAAGTGGA | | |
| | AAAGCTAT | | |
| 625339_25353836_396997_ | TGTAATTCTATATCTTCAGTAATAC | 120 | 917 |
| HPV35REF.1_1_86 | TACTTAAATCCTGATAATAATGTAC | | |
| | CCGTGCCCCTATAGCTTTTCCACTT | | |
| | CGTGTATGCATAG | | |
| | TACGTTTATTACCTACTCTACTATA | | |
| | TCTAATA | | |
| 625339_25353836_396997_ | AGGGGCACGGGTACATTATTATCAG | 120 | 918 |
| HPV35REF.1_1_87 | GATTTAAGTAGTATTACTGAAGATA | | |
| | TAGAATTACAACCCTTACAACATGT | | |
| | ACCATCCTCTTTA | | |
| | CCACATACCACTGTTTCAACATCAT | | |
| | TAAATGA | | |
| 625339_25353836_396997_ | GATGCTGAAAATATAATATCTTCCT | 120 | 919 |
| HPV35REF.1_1_88 | CAGTATCTATAGGAGCATAAATATC | | |
| | AAACATACCATCATTTAATGATGTT | | |
| | GAAACAGTGGTAT | | |
| | GTGGTAAAGAGGATGGTACATGTTG | | |
| | TAAGGGT | | |
| 625339_25353836_396997_ | TGGTATGTTTGATATTTATGCTCCT | 120 | 920 |
| HPV35REF.1_1_89 | ATAGATACTGAGGAAGATATTATAT | | |
| | TTTCAGCATCTTCTAACAATACTTT | | |
| | ATATACTACATCTA | | |
| | ACACTGCATATGTTCCTAGCAATAC | | |
| | TACTAT | | |
| 625339_25353836_396997_ | GAGTTAAATACAATGTCTGGCCCTG | 120 | 921 |
| HPV35REF.1_1_90 | CTGTTATAGGAATATCATAGCCACT | | |
| | ACTTAATGGTATAGTAGTATTGCTA | | |
| | GGAACATATGCA | | |
| | GTGTTAGATGTAGTATATAAAGTAT | | |
| | TGTTAGAA | | |
| 625339_25353836_396997_ | ACCATTAAGTAGTGGCTATGATATT | 120 | 922 |
| HPV35REF.1_1_91 | CCTATAACAGCAGGGCCAGACATTG | | |
| | TATTTAACTCTAATACTATTACTAA | | |
| | CACTGTACTACCG | | |
| | GTACCCACAGGTCCTATATATTCTA | | |
| | TTATTGC | | |
| 625339_25353836_396997_ | CGTTTACGACGTCGTTTTAATAAAT | 120 | 923 |
| HPV35REF.1_1_92 | AATAACTAGGGTGTAAATAAAAGTC | | |
| | ACCCCCATCTGCAATAATAGAATAT | | |
| | ATAGGACCTGTG | | |
| | GGTACCGGTAGTACAGTGTTAGTAA | | |
| | TAGTATTA | | |
| 625339_25353836_396997_ | AGATGGGGGTGACTTTTATTTACAC | 120 | 924 |
| HPV35REF.1_1_93 | CCTAGTTATTATTTATTAAAACGAC | | |
| | GTCGTAAACGTATCCCATATTTTTT | | |
| | TGCAGATGTCTCT | | |
| | GTGGCGGTCTAACGAAGCCACTGTC | | |
| | TACCTGC | | |
| 625339_25353836_396997_ | AGATGTTTGTGCGTGTTACATATTC | 120 | 925 |
| HPV35REF.1_1_94 | ATCAGTGCTAACAACCTTAGACACT | | |
| | GACACTGGAGGCAGGTAGACAGTGG | | |
| | CTTCGTTAGAC | | |
| | CGCCACAGAGACATCTGCAAAAAAA | | |
| | TATGGGATA | | |
| 625339_25353836_396997_ | CTCCAGTGTCAGTGTCTAAGGTTGT | 120 | 926 |
| HPV35REF.1_1_95 | TAGCACTGATGAATATGTAACACGC | | |
| | ACAAACATCTACTATCATGCAGGCA | | |
| | GTTCTAGGCTAT | | |
| | TAGCTGTGGGTCACCCATACTATGC | | |
| | TATTAAAA | | |
| 625339_25353836_396997_ | TAAATACTCTGTATTGCAAACCAGA | 120 | 927 |
| HPV35REF.1_1_96 | TACCTTGGGTACTGCTATTTTATTA | | |
| | GAATCTTGTTTTTTAATAGCATAGT | | |
| | ATGGGTGACCCAC | | |
| | AGCTAATAGCCTAGAACTGCCTGCA | | |
| | TGATAGT | | |
| 625339_25353836_396997_ | AACAAGATTCTAATAAAATAGCAGT | 120 | 928 |
| HPV35REF.1_1_97 | ACCCAAGGTATCTGGTTTGCAATAC | | |
| | AGAGTATTTAGAGTAAAATTACCAG | | |
| | ATCCTAATAAGTT | | |
| | TGGATTTCCAGACACATCATTTTAT | | |
| | GATCCTG | | |
| 625339_25353836_396997_ | CCAATGGCTGACCACGACCTACTTC | 120 | 929 |
| HPV35REF.1_1_98 | AACTCCTGTACAGGCCCAAACCAAA | | |
| | CGCTGGGAGGCAGGATCATAAAATG | | |
| | ATGTGTCTGGA | | |
| | AATCCAAACTTATTAGGATCTGGTA | | |
| | ATTTTACTC | | |
| 625339_25353836_396997_ | CCTCCCAGCGTTTGGTTTGGGCCTG | 120 | 930 |
| HPV35REF.1_1_99 | TACAGGAGTTGAAGTAGGTCGTGGT | | |
| | CAGCCATTGGGTGTAGGTATTAGTG | | |
| | GTCATCCTTT | | |
| | ATTAAATAAATTGGATGATACTGAA | | |
| | AATTCTAATA | | |
| 625339_25353836_396997_ | TTTGTTTATAATCCATAGAAATGCA | 120 | 931 |
| HPV35REF.1_1_100 | TTCCCTGTTATCTGTACCAGAGTTA | | |
| | CCAACATATTTATTAGAATTTTCAG | | |
| | TATCATCCAATTTA | | |
| | TTTAATAAAGGATGACCACTAATAC | | |
| | CTACAC | | |
| 625339_25353836_396997_ | AATATGTTGGTAACTCTGGTACAGA | 120 | 932 |
| HPV35REF.1_1_101 | TAACAGGGAATGCATTTCTATGGAT | | |
| | TATAAACAAACACAATTGTGTTTAA | | |
| | TAGGTTGTAGGC | | |
| | CTCCTATAGGTGAACATTGGGGAAA | | |
| | AGGCACAC | | |
| 625339_25353836_396997_ | CAGTGTTTAGTAACTCCAAAGGAGG | 120 | 933 |
| HPV35REF.1_1_102 | ACATTCTCCTGCTTTTACCTGGTTA | | |
| | GCATTACAAGGTGTGCCTTTTCCCC | | |
| | AATGTTCACCTA | | |
| | TAGGAGGCCTACAACCTATTAAACA | | |
| | CAATTGTG | | |
| 625339_25353836_396997_ | CTTGTAATGCTAACCAGGTAAAAGC | 120 | 934 |
| HPV35REF.1_1_103 | AGGAGAATGTCCTCCTTTGGAGTTA | | |
| | CTAAACACTGTACTACAAGACGGGG | | |
| | ACATGGTAGAC | | |
| | ACAGGATTTGGTGCAATGGATTTTA | | |
| | CTACATTAC | | |
| 625339_25353836_396997_ | AATCAGGATATTTGCAAATGGAACT | 120 | 935 |
| HPV35REF.1_1_104 | GCATATATCTAGGGGAACATCACTT | | |
| | TTATTAGCTTGTAATGTAGTAAAAT | | |
| | CCATTGCACCAAA | | |
| | TCCTGTGTCTACCATGTCCCCGTCT | | |
| | TGTAGTA | | |
| 625339_25353836_396997_ | AAGCTAATAAAAGTGATGTTCCCCT | 120 | 936 |
| HPV35REF.1_1_105 | AGATATATGCAGTTCCATTTGCAAA | | |
| | TATCCTGATTATCTAAAAATGGTTT | | |
| | CTGAGCCATATGG | | |
| | AGATATGTTATTTTTTTATTTACGT | | |
| | AGGGAGC | | |
| 625339_25353836_396997_ | CTGCAGGTACTGTTTCACCTACAGT | 120 | 937 |
| HPV35REF.1_1_106 | TCCAGCCCTATTAAATAAATGTCTA | | |
| | ACAAACATTTGCTCCCTACGTAAAT | | |
| | AAAAAAATAACAT | | |
| | ATCTCCATATGGCTCAGAAACCATT | | |
| | TTTAGAT | | |
| 625339_25353836_396997_ | AAATGTTTGTTAGACATTTATTTAA | 120 | 938 |
| HPV35REF.1_1_107 | TAGGGCTGGAACTGTAGGTGAAACA | | |
| | GTACCTGCAGACCTATATATTAAGG | | |
| | GTACCACTGGCA | | |
| | CATTGCCTAGTACTAGTTATTTTCC | | |
| | TACTCCTA | | |
| 625339_25353836_396997_ | CACGTTGCAACCAATATGGTTTATT | 120 | 939 |
| HPV35REF.1_1_108 | AAATATTTGTGCATCGGAGGTTACC | | |
| | ATAGAGCCACTAGGAGTAGGAAAAT | | |
| | AACTAGTACTAG | | |
| | GCAATGTGCCAGTGGTACCCTTAAT | | |
| | ATATAGGT | | |
| 625339_25353836_396997_ | GTGGCTCTATGGTAACCTCCGATGC | 120 | 940 |
| HPV35REF.1_1_109 | ACAAATATTTAATAAACCATATTGG | | |
| | TTGCAACGTGCACAAGGCCATAATA | | |
| | ATGGTATTTGTT | | |
| | GGAGTAACCAATTGTTTGTTACTGT | | |
| | AGTTGATA | | |
| 625339_25353836_396997_ | TATATGTACTGTCACTAGAAGACAC | 120 | 941 |
| HPV35REF.1_1_110 | AGCAGAACACACAGACATATTTGTA | | |
| | CTACGGGTTGTATCAACTACAGTAA | | |
| | CAAACAATTGGT | | |
| | TACTCCAACAAATACCATTATTATG | | |
| | GCCTTGTG | | |
| 625339_25353836_396997_ | CAACCCGTAGTACAAATATGTCTGT | 120 | 942 |
| HPV35REF.1_1_111 | GTGTTCTGCTGTGTCTTCTAGTGAC | | |
| | AGTACATATAAAAATGACAATTTTA | | |
| | AGGAATATTTAAG | | |
| | GCATGGTGAAGAATATGATTTACAG | | |
| | TTTATTT | | |
| 625339_25353836_396997_ | GGTTCATACTATGAATATATGTCAT | 120 | 943 |
| HPV35REF.1_1_112 | AACATCTGCTGTTAGTGTTATTTTA | | |
| | CATAACTGAAAAATAAACTGTAAAT | | |
| | CATATTCTTCACCA | | |
| | TGCCTTAAATATTCCTTAAAATTGT | | |
| | CATTTT | | |
| 625339_25353836_396997_ | TTCAGTTATGTAAAATAACACTAAC | 120 | 944 |
| HPV35REF.1_1_113 | AGCAGATGTTATGACATATATTCAT | | |
| | AGTATGAACCCGTCCATTTTAGAGG | | |
| | ATTGGAATTTTGG | | |
| | CCTTACACCACCGCCTTCTGGTACC | | |
| | TTAGAGG | | |
| 625339_25353836_396997_ | GTTTTGGTGCACTGGGTTTTTGACA | 120 | 945 |
| HPV35REF.1_1_114 | AGTTACAGCCTGTGATGTTACATAG | | |
| | CGATATGTGTCCTCTAAGGTACCAG | | |
| | AAGGCGGTGGT | | |
| | GTAAGGCCAAAATTCCAATCCTCTA | | |
| | AAATGGACG | | |
| 625339_25353836_396997_ | ACACATATCGCTATGTAACATCACA | 120 | 946 |
| HPV35REF.1_1_115 | GGCTGTAACTTGTCAAAAACCCAGT | | |
| | GCACCAAAACCTAAAGATGATCCAT | | |
| | TAAAAAATTATAC | | |
| | TTTTTGGGAGGTTGATTTAAAGGAA | | |
| | AAGTTTT | | |
| 625339_25353836_396997_ | CCTTTAGTCCTGCTTGTAACAAAAA | 120 | 947 |
| HPV35REF.1_1_116 | TTTACGGCCCAACGGAAATTGATCT | | |
| | AAGTCTGCAGAAAACTTTTCCTTTA | | |
| | AATCAACCTCCCA | | |
| | AAAAGTATAATHTTTAATGGATGAT | | |
| | GTTTAG | | |
| 625339_25353836_396997_ | CTGCAGACTTAGATCAATTTCCGTT | 120 | 948 |
| HPV35REF.1_1_117 | GGGCCGTAAATTTTTGTTACAAGCA | | |
| | GGACTAAAGGCCAGGCCTAATTTTA | | |
| | GATTAGGCAAGC | | |
| | GTGCAGCTCCAGCATCTACATCTAA | | |
| | AAAATCTT | | |
| 625339_25353836_396997_ | CACACAGTATACATGCATACACATT | 120 | 949 |
| HPV35REF.1_1_118 | TACACATTAACTTTTTACTTTTCTA | | |
| | CGTTTAGTAGAAGATTTTTTAGATG | | |
| | TAGATGCTGGAGC | | |
| | TGCACGCTTGCCTAATCTAAAATTA | | |
| | GGCCTGG | | |
| 625339_25353836_396997_ | CTACTAAACGTAGAAAAGTAAAAAG | 120 | 950 |
| HPV35REF.1_1_119 | TTAAtgtgtaaatgtgtatgcatgt | | |
| | atactgtgtgttatgtgttgtagtg | | |
| | cttgtatatatattatgtgttgtgg | | |
| | tgcctgtttgtgttgtACAT | | |
| 625339_25353836_396997_ | ACATACAAAAAACACCCACGTACAA | 120 | 951 |
| HPV35REF.1_1_120 | CACATTGCACAATATTATACACACA | | |
| | TTTACACGCCATGTACAACACAAAC | | |
| | AGGCACCACAACA | | |
| | CATAATATATATACAAGCACTACAA | | |
| | CACATAA | | |
| 625339_25353836_396997_ | GGCGTGTAAATGTGTGTATAATATT | 120 | 952 |
| HPV35REF.1_1_121 | GTGCAATGTGTTGTACGTGGGTGTT | | |
| | TTTtgtatgtatgttgttgtatgta | | |
| | tgtCAGTACGCAATAAAAGTGA | | |
| | TGTGTGTGTTTATAATTAACACT | | |
| 625339_25353836_396997_ | TATAGCGTGTACTGTAATTATGTAA | 120 | 953 |
| HPV35REF.1_1_122 | GTCATATGGGTGCACCCATAGTCAT | | |
| | ACAACAATACAGTGTTAATTATAAA | | |
| | CACACACATCACT | | |
| | TTTATTGCGTACTGACATACATACA | | |
| | ACAACAT | | |
| 625339_25353836_396997_ | GTATTGTTGTATGACTATGGGTGCA | 120 | 954 |
| HPV35REF.1_1_123 | CCCATATGACTTACATAATTACAGT | | |
| | ACACGCTATATGTTGTATATAACAA | | |
| | TTCTACCTCCATT | | |
| | TTGTGTGTTAGTGTCCTTTACATTA | | |
| | CCTTTCA | | |
| 625339_25353836_396997_ | TGAGGTAGGAATGTTTTTGTAAAAA | 120 | 955 |
| HPV35REF.1_1_124 | ACATATAAAGCTTGCCAACAGCAAC | | |
| | CGAAATCGGTTGAAAGGTAATGTAA | | |
| | AGGACACTAACA | | |
| | CACAAAATGGAGGTAGAATTGTTAT | | |
| | ATACAACA | | |
| 625339_25353836_396997_ | ACCGATTTCGGTTGCTGTTGGCAAG | 120 | 956 |
| HPV35REF.1_1_125 | CTTTATATGTTTTTTACAAAAACAT | | |
| | TCCTACCTCAGCAGAACACTTAATC | | |
| | CTTGTGTTCCTGA | | |
| | TATATATTGTTTGCCAACTTTATAT | | |
| | TGGCTTT | | |
| 625339_25353836_396997_ | TGGACCACAAGTATGAAAAATGACT | 120 | 957 |
| HPV35REF.1_1_126 | AATACTGCAAGATGAATCAAGTTTA | | |
| | AAGATTGGCAAAAGCCAATATAAAG | | |
| | TTGGCAAACAAT | | |
| | ATATATCAGGAACACAAGGATTAAG | | |
| | TGTTCTGC | | |
| 625339_25353836_396997_ | TGCCAATCTTTAAACTTGATTCATC | 120 | 958 |
| HPV35REF.1_1_127 | TTGCAGTATTAGTCATTTTTCATAC | | |
| | TTGTGGTCCACCCACACTTGTAACA | | |
| | CTTGTAACAGTGC | | |
| | TTTTAGGCACATATTTTTTGCATTT | | |
| | CTAAAGG | | |
| 625339_25353836_396997_ | ATGTGTAGGGTGGTGTTGGCAAACA | 120 | 959 |
| HPV35REF.1_1_128 | CACATAATATGTAAAGCCAAGTGTG | | |
| | CAATTAAAGCCCTTTAGAAATGCAA | | |
| | AAAATATGTGCC | | |
| | TAAAAGCACTGTTACAAGTGTTACA | | |
| | AGTGTGGG | | |
| 625339_25353836_396997_ | GCTTTAATTGCACACTTGGCTTTAC | 120 | 960 |
| HPV35REF.1_1_129 | ATATTATGTGTGTTTGCCAACACCA | | |
| | CCCTACACATCCTGCCAACTTTAAG | | |
| | TTAAAACATGCAT | | |
| | GTAAAACATTACTCACTGTATTACA | | |
| | CATTGTT | | |
| 625339_25353836_396997_ | ATAAAAAGAAATGCTTATAAAACTG | 120 | 961 |
| HPV35REF.1_1_130 | TAATCCAAATCGGTTGGACACACCT | | |
| | GTGTGCATATAACAATGTGTAATAC | | |
| | AGTGAGTAATGTT | | |
| | TTACATGCATGTTTTAACTTAAAGT | | |
| | TGGCAGG | | |

| Human_12gene_ | | | SEQ |
|---|---|---|---|
| panel_SequenceName | Sequence | Length | ID#: |
| 186573_9856646_PTEN.1_1 | TCAGCCACAGGCTCCCAGACATGAC | 120 | 962 |
| | AGCCATCATCAAAGAGATCGTTAGC | | |
| | AGAAACAAAAGGAGATATCAAGAGG | | |
| | ATGGATTCGACTTAGACTTGACCTG | | |
| | TATCCATTTCTGCGGCTGCT | | |
| 186573_9856647_PTEN_1 | CTTAACTAAAGTACTCAGATATTTA | 120 | 963 |
| | TCCAAACATTATTGCTATGGGATTT | | |
| | CCTGCAGAAAGACTTGAAGGCGTAT | | |
| | ACAGGAACAATATTGATGATGTAGT | | |
| | AAGGTAAGAATGCTTTGATT | | |
| 185573_9856648_PTEN_1 | GTTAATGGTGGCTTTTTGTTTGTTT | 120 | 964 |
| | GTTTTGTTTTAAGGTTTTTGGATTC | | |
| | AAAGCATAAAAACCATTACAAGATA | | |
| | TACAATCTGTAAGTATGTTTTCTTA | | |
| | TTTGTATGCTTGCAAATATC | | |
| 186573_9856649_PTEN_1 | AAAGATAACTTTATATCACTTTTAA | 120 | 965 |
| | ACTTTTCTTTTAGTTGTGCTGAAAG | | |
| | ACATTATGACACCGCCAAATTTAAT | | |
| | TGCAGAGGTAGGTATGAATGTACTG | | |
| | TACTATGTTGTATAACTTAA | | |
| 186573_9856650_PTEN.1_1 | TTGCACAATATCCTTTTGAAGACCA | 120 | yoo |
| | TAACCCACCACAGCTAGAACTTATC | | |
| | AAACCCTTTTGTGAAGATCTTGACC | | |
| | AATGGCTAAGTGAAGATGACAATCA | | |
| | TGTTGCAGCAATTCACTGTA | | |
| 186573_9856650_PTEN.1_2 | AAGCTGGAAAGGGACGAACTGGTGT | 120 | 967 |
| | AATGATATGTGCATATTTATTACAT | | |
| | CGGGGCAAATTTTTAAAGGCACAAG | | |
| | AGGCCCTAGATTTCTATGGGGAAGT | | |
| | AAGGACCAGAGACAAAAAGG | | |
| 186573_9856651_PTEN_1 | ATGGAACATTTTTTTTCAATTTGGC | 120 | 968 |
| | TTCTCTTTTTTTTCTGTCCACCAGG | | |
| | GAGTAACTATTCCCAGTCAGAGGCG | | |
| | CTATGTGTATTATTATAGCTACCTG | | |
| | TTAAAGAATCATCTGGATTA | | |
| 186573_9856651_PTEN_2 | TAGACCAGTGGCACTGTTGTTTCAC | 120 | 969 |
| | AAGATGATGTTTGAAACTATTCCAA | | |
| | TGTTCAGTGGCGGAACTTGCAGTAA | | |
| | GTGCTTGAAATTCTCATCCTTCCAT | | |
| | GTATTGGAACAGTTTTCTTA | | |
| 18S573_9856652_PTEN_1 | CTGTGAAATAATACTGGTATGTATT | 120 | 970 |
| | TAACCATGCAGATCCTCAGTTTGTG | | |
| | GTCTGCCAGCTAAAGGTGAAGATAT | | |
| | ATTCCTCCAATTCAGGACCCACACG | | |
| | ACGGGAAGACAAGTTCATGT | | |
| 186573_9856652_PTEN_2 | ACTTTGAGTTCCCTCAGCCGTTACC | 120 | 971 |
| | TGTGTGTGGTGATATCAAAGTAGAG | | |
| | TTCTTCCACAAACAGAACAAGATGC | | |
| | TAAAAAAGGTTTGTACTTTACTTTC | | |
| | ATTGGGAGAAATATCCAAAA | | |
| 186573_9856653_PTEN.1_1 | TTTTTAGGACAAAATGTTTCACTTT | 120 | 972 |
| | TGGGTAAATACATTCTTCATACCAG | | |
| | GACCAGAGGAAACCTCAGAAAAAGT | | |
| | AGAAAATGGAAGTCTATGTGATCAA | | |
| | GAAATCGATAGCATTTGCAG | | |
| 186573_9856653_PTEN.1_2 | TATAGAGCGTGCAGATAATGACAAG | 120 | 973 |
| | GAATATCTAGTACTTACTTTAACAA | | |
| | AAAATGATCTTGACAAAGCAAATAA | | |
| | AGACAAAGCCAACCGATACTTTTCT | | |
| | CCAAATTTTAAGGTCAGTTA | | |
| 185573_9856654_PTEN_1 | TTTTCATTTTAAATTTTCTTTCTCT | 120 | 974 |
| | AGGTGAAGCTGTACTTCACAAAAAC | | |
| | AGTAGAGGAGCCGTCAAATCCAGAG | | |
| | GCTAGCAGTTCAACTTCTGTAACAC | | |
| | CAGATGTTAGTGACAATGAA | | |
| 186573_9856654_PTEN_2 | CCTGATCATTATAGATATTCTGACA | 120 | 975 |
| | CCACTGACTCTGATCCAGAGAATGA | | |
| | ACCTTTTGATGAAGATCAGCATACA | | |
| | CAAATTACAAAAGTCTGAATTTTTT | | |
| | TTTATCAAGAGGGATAAAAC | | |
| 186573_9856655_HRAS_1 | CGGGGAGCCGGGGTCATCCGGTGGG | 120 | 976 |
| | CGTGGCGGCCGCCCTGGGAGTCCCC | | |
| | CTCACCTGCGTCAGGAGAGCACACA | | |
| | CTTGCAGCTCATGCAGCCGGGGCCA | | |
| | CTCTCATCAGGAGGGTTCAG | | |
| 185573_9856655_HRAS_2 | CTTCCGCAGCTTGTGCTGCCGGATC | 120 | 977 |
| | TCACGCACCAACGTGTAGAAGGCAT | | |
| | CCTCCACTCCCTGGGAAAGGAGGGA | | |
| | TGGGATCAGGAGGGACCGGCCTGTG | | |
| | GCCGCCTGCCTGGGTGAGGG | | |
| 185573_9656656_HRAS_1 | ACAGCGCGAGGGGCCGCTGGGTCAC | 120 | 978 |
| | ATGGGTCCCGGGGGGTCCCAGAGGG | | |
| | TCCCGGAGCTGGAGCTAGAGCCAGA | | |
| | GCGGCTGCCCTGTGTCAAGGGAGAG | | |
| | GGTCAGTGAGTGCTGCTCCC | | |
| 186573_9856656_HRAS_2 | TGGCTGGGGCGGGGCGGGGCGGGTC | 120 | 979 |
| | CCTGGCTAGCTGTGGGGTGGAGAGC | | |
| | TGCCTCACCTGCCGGGTCTTGGCCG | | |
| | AGGTCTCGATGTAGGGGATGCCGTA | | |
| | GCTTCGGGCGAGGTCCTGAG | | |
| 186573_9856656_HRAS_3 | CCTGCCGAGATTCCACAGTGCGTGC | 120 | 980 |
| | AGCCAGGTCACACTTGTTCCCCACC | | |
| | AGCACCATGGGCACGTCATCCGAGT | | |
| | CCTTCACCCGTTTGATCTGCTCCCT | | |
| | GAGAGGTGGAAAGCGAGAGC | | |
| 185573_9856657_HRAS_1 | GGCTCCCGGGCCAGCCTCACGGGGT | 120 | 981 |
| | TCACCTGTACTGGTGGATGTCCTCA | | |
| | AAAGACTTGGTGTTGTTGATGGCAA | | |
| | ACACACACAGGAAGCCCTCCCCGGT | | |
| | GCGCATGTACTGGTCCCGCA | | |
| 186573_9856657_HRAS_2 | TGGCGCTGTACTCCTCCTGGCCGGC | 120 | 982 |
| | GGTATCCAGGATGTCCAACAGGCAC | | |
| | GTCTCCCCATCAATGACCACCTGCT | | |
| | TCCGGTAGGAATCCTGCAGGAGGAC | | |
| | AGGGCTCAGGGACCCCCTCA | | |
| 186573_9856658_HRAS.1_1 | TCACCTCTATAGTGGGGTCGTATTC | 120 | 983 |
| | GTCCACAAAATGGTTCTGGATCAGC | | |
| | TGGATGGTCAGCGCACTCTTGCCCA | | |
| | CACCGCCGGCGCCCACCACCACCAG | | |
| | CTTATATTCCGTCATCGCTC | | |
| 186573_9856659_TP53_1 | AACAAGAAGTGGAGAATGTCAGTCT | 120 | 984 |
| | GAGTCAGGCCCTTCTGTCTTGAACA | | |
| | TGAGTTTTTTATGGCGGGAGGTAGA | | |
| | CTGACCCTTTTTGGACTTCAGGTGG | | |
| | CTGTAGGAGACAGAAGCAGG | | |
| 186573_9856660_TP53_1 | ACTCACCTGGAGTGAGCCCTGCTCC | 120 | 985 |
| | CCCCTGGCTCCTTCCCAGCCTGGGC | | |
| | ATCCTTGAGTTCCAAGGCCTCATTC | | |
| | AGCTCTCGGAACATCTCGAAGCGCT | | |
| | CACGCCCACGGATCTGCAGC | | |
| 186573_9856661_TP531_1 | CGTAAGTCAAGTAGCATCTGTATCA | 120 | 986 |
| | GGCAAAGTCATAGAACCATTTTCAT | | |
| | GCTCTCTTTAACAATTTTCTTTTTG | | |
| | AAAGCTGGTCTGGTCCTTTAAAATA | | |
| | TATATTATGGTATAAGTTGG | | |
| 186573.9856662.TP53.1 | TAAGAGGTCCCAAGACTTAGTACCT | 120 | 987 |
| | GAAGGGTGAAATATTCTCCATCCAG | | |
| | TGGTTTCTTCTTTGGCTGGGGAGAG | | |
| | GAGCTGGTGTTGTTGGGCAGTGCTA | | |
| | GGAAAGAGGCAAGGAAAGGT | | |
| 185573.9856663.TP53.1 | GGCATAACTGCACCCTTGGTCTCCT | 120 | 988 |
| | CCACCGCTTCTTGTCCTGCTTGCTT | | |
| | ACCTCGCTTAGTGCTCCCTGGGGGC | | |
| | AGCTCGTGGTGAGGCTCCCCTTTCT | | |
| | TGCGGAGATTCTCTTCCTCT | | |
| 186573_9856663_TP53_2 | GTGCGCCGGTCTCTCCCAGGACAGG | 120 | 989 |
| | CACAAACACGCACCTCAAAGCTGTT | | |
| | CCGTCCCAGTAGATTACCACTACTC | | |
| | AGGATAGGAAAAGAGAAGCAAGAGG | | |
| | CAGTAAGGAAATCAGGTCCT | | |
| 186573_9856664_TP531_1 | CTGACCTGGAGTCTTCCAGTGTGAT | 120 | 990 |
| | GATGGTGAGGATGGGCCTCCGGTTC | | |
| | ATGCCGCCCATGCAGGAACTGTTAC | | |
| | ACATGTAGTTGTAGTGGATGGTGGT | | |
| | ACAGTCAGAGCCAACCTAGG | | |
| 186573.9856665.TP53.1 | AGACCTCAGGCGGCTCATAGGGCAC | 120 | 991 |
| | CACCACACTATGTCGAAAAGTGTTT | | |
| | CTGTCATCCAAATACTCCACACGCA | | |
| | AATTTCCTTCCACTCGGATAAGATG | | |
| | CTGAGGAGGGGCCAGACCTA | | |
| 186573_985666G_TP53_1 | CTGTCGTCTCTCCAGCCCCAGCTGC | 120 | 992 |
| | TCACCATCGCTATCTGAGCAGCGCT | | |
| | CATGGTGGGGGCAGCGCCTCACAAC | | |
| | CTCCGTCATGTGCTGTGACTGCTTG | | |
| | TAGATGGCCATGGCGCGGAC | | |
| 186573.9856666.TP53.2 | GCGGGTGCCGGGCGGGGGTGTGGAA | 120 | 993 |
| | TCAACCCACAGCTGCACAGGGCAGG | | |
| | TCTTGGCCAGTTGGCAAAACATCTT | | |
| | GTTGAGGGCAGGGGAGTACTGTAGG | | |
| | AAGAGGAAGGAGACAGAGTT | | |
| 186573_9856667_TPS3_1 | CCAGGCATTGAAGTCTCATGGAAGC | 120 | 994 |
| | CAGCCCCTCAGGGCAACTGACCGTG | | |
| | CAAGTCACAGACTTGGCTGTCCCAG | | |
| | AATGCAAGAAGCCCAGACGGAAACC | | |
| | GTAGCTGCCCTGGTAGGTTT | | |
| 18S573_9856667_TP53_2 | TCTGGGAAGGGACAGAAGATGACAG | 120 | 995 |
| | GGGCCAGGAGGGGGCTGGTGCAGGG | | |
| | GCCGCCGGTGTAGGAGCTGCTGGTG | | |
| | CAGGGGCCACGGGGGGAGCAGCCTC | | |
| | TGGCATTCTGGGAGCTTCAT | | |
| 186573_9856667_TP53_3 | CTGGACCTGGGTCTTCAGTGAACCA | 120 | 996 |
| | TTGTTCAATATCGTCCGGGGACAGC | | |
| | ATCAAATCATCCATTGCTTGGGACG | | |
| | GCAAGGGGGACTGTAGATGGGTGAA | | |
| | AAGAGCAGTCAGAGGACCAG | | |
| 186573.9856668TP531.1 | CCTCCAGGTCCCCAGCCCTCCAGGT | 120 | 997 |
| | CCCCAGCCCAACCCTTGTCCTTACC | | |
| | AGAACGTTGTTTTCAGGAAGTCTGA | | |
| | AAGACAAGAGCAGAAAGTCAGTCCC | | |
| | ATGGAATTTTCGCTTCCCAC | | |
| 185573.9856669.TP53.1.1 | CCCTTCCAATGGATCCACTCACAGT | 120 | 998 |
| | TTCCATAGGTCTGAAAATGTTTCCT | | |
| | GACTCAGAGGGGGCTCGACGCTAGG | | |
| | ATCTGACTGCGGCTCCTCCATGGCA | | |
| | GTGACCCGGAAGGCAGTCTG | | |
| 18S573.9856670.NFE2L2_1 | GGATGTGCTGGGCTGGCTGAATTGG | 120 | 999 |
| | GAGAAATTCACCTGTCTCTTCATCT | | |
| | AGTTGTAACTGAGCGAAAAAGGCTT | | |
| | TCTCTTGCTCCTTTTGGAGTTGTTC | | |
| | TTGTCTTTCCTTTTCAAGTT | | |
| 186573_9856670_NFE2L2_2 | TTTTCTGTTTTTCCAGCTCATACTC | 120 | 1000 |
| | TTTCCGTCGCTGACTGAAGTCAAAT | | |
| | ACTTCTCGACTTACTCCAAGATCTA | | |
| | TATCTTGCCTCCAAAGTATGTCAAT | | |
| | CAAATCCATGTCCTATGTTT | | |
| 186573.9856671_CASP8_1 | ACTTAGATTATATTCTCCTGCCTTT | 120 | 1001 |
| | TAAAAAGATGGACTTCAGCAGAAAT | | |
| | CTTTATGATATTGGGGAACAACTGG | | |
| | ACAGTGAAGATCTGGCCTCCCTCAA | | |
| | GTTCCTGAGCCTGGACTACA | | |
| 185573_9856671_CASP8_2 | TTCCGCAAAGGAAGCAAGAACCCAT | 120 | 1002 |
| | CAAGGATGCCTTGATGTTATTCCAG | | |
| | AGACTCCAGGAAAAGAGAATGTTGG | | |
| | AGGAAAGCAATCTGTCCTTCCTGAA | | |
| | GGAGCTGCTCTTCCGAATTA | | |
| 186573.9856671_CASP8_3 | ATAGACTGGATTTGCTGATTACCTA | 120 | 1003 |
| | CCTAAACACTAGAAAGGAGGAGATG | | |
| | GAAAGGGAACTTCAGACACCAGGCA | | |
| | GGGCTCAAATTTCTGCCTACAGGTG | | |
| | GGTGGAAACTCCCATTGTGG | | |
| 186573.9856672.CASP81_1 | TCTGTGAATTACTGTGGTATAACGT | 120 | 1004 |
| | GACTGTTCAAATTTCACTTTTCAGG | | |
| | GGCTTTGACCACGACCTTTGAAGAG | | |
| | CTTCATTTTGAGATCAAGCCCCACG | | |
| | ATGACTGCACAGTAGAGCAA | | |
| 186573.9856672.CASP8.1_2 | ATCTATGAGATTTTGAAAATCTACC | 120 | 1005 |
| | AACTCATGGACCACAGTAACATGGA | | |
| | CTGCTTCATCTGCTGTATCCTCTCC | | |
| | CATGGAGACAAGGGCATCATCTATG | | |
| | GCACTGATGGACAGGAGGCC | | |
| 186573.9856672.CASP8.1_3 | CCCATCTATGAGCTGACATCTCAGT | 120 | 1006 |
| | TCACTGGTTTGAAGTGCCCTTCCCT | | |
| | TGCTGGAAAACCCAAAGTGTTTTTT | | |
| | ATTCAGGCTTGTCAGGGGGATAACT | | |
| | ACCAGAAAGGTATACCTGTT | | |
| 186573.9856672_CASP8.1_4 | GAGACTGATTCAGAGGAGCAACCCT | 120 | 1007 |
| | ATTTAGAAATGGATTTATCATCACC | | |
| | TCAAACGAGATATATCCCGGATGAG | | |
| | GCTGACTTTCTGCTGGGGATGGCCA | | |
| | CTGTGAATAACTGTGTTTCC | | |
| 186573.9856672.CASP8.1_5 | TACCGAAACCCTGCAGAGGGAACCT | 120 | 1008 |
| | GGTACATCCAGTCACTTTGCCAGAG | | |
| | CCTGAGAGAGCGATGTCCTCGGTAA | | |
| | GTTTTGCCTACTCAGCCCTCCTCAC | | |
| | TGTTACACTACCTTCCCCCC | | |
| 186573.9856673.CASP8.1_1 | GCACAGCAGAGGAGACAGTTCATCA | 120 | 1009 |
| | GTTGCTTTCCCCCACAGACAGTCAC | | |
| | AATATTATGTGATGTATTTCAGAGG | | |
| | CGATGATATTCTCACCATCCTGACT | | |
| | GAAGTGAACTATGAAGTAAG | | |
| 186573.9856673.CASP8.1_2 | CAACAAGGATGACAAGAAAAACATG | 119 | 1010 |
| | GGGAAACAGATGCCTCAGCCTACTT | | |
| | TCACACTAAGAAAAAAACTTGTCTT | | |
| | CCCTTCTGATTGATGGTGCTATTTT | | |
| | GTTTGTTTTGTTTTGTTTT | | |
| 186573.9856674.MAPK1_1 | CTTACCTCGTCACTCGGGTCGTAAT | 120 | 1011 |
| | ACTGCTCCAGATATGGGTGGGCCAG | | |
| | AGCCTGTTCTACTTCAATCCTCTTG | | |
| | TGTGGGTTGAATGTCAACATTTTGT | | |
| | CCAATAAGTCCAGAGCTATA | | |
| 186573.9856675.EP300_1 | TAGGAGAGTATACATATCTTACCTC | 120 | 1012 |
| | GATAGTGTTCATTTCTTCCGTCCTA | | |
| | AATGCTTGAGGACTGCAGTCTATCA | | |
| | TGAAATCCTAATTGGATATTTAGAA | | |
| | TATGTCAAGAAATTAGGGTA | | |
| 186573.9856676.PIK3CA_1 | AACAATGCCTCCACGACCATCATCA | 120 | 1013 |
| | GGTGAACTGTGGGGCATCCACTTGA | | |
| | TGCCCCCAAGAATCCTAGTAGAATG | | |
| | TTTACTACCAAATGGAATGATAGTG | | |
| | ACTTTAGAATGCCTCCGTGA | | |
| 186573.9856676.PIK3CA_2 | GGCTACATTAATAACCATAAAGCAT | 120 | 1014 |
| | GAACTATTTAAAGAAGCAAGAAAAT | | |
| | ACCCCCTCCATCAACTTCTTCAAGA | | |
| | TGAATCTTCTTACATTTTCGTAAGT | | |
| | GTTACTCAAGAAGCAGAAAG | | |
| 186573_9856676_PIK3CA_3 | GGAAGAATTTTTTGATGAAACAAGA | 120 | 1015 |
| | CGACTTTGTGACCTTCGGCTTTTTC | | |
| | AACCCTTTTTAAAAGTAATTGAACC | | |
| | AGTAGGCAACCGTGAAGAAAAGATC | | |
| | CTCAATCGAGAAATTGGTAT | | |
| 186573.9856677_PIK3CA.1_1 | GTAATTTTATTAAAGGTTTTGCTAT | 120 | 1016 |
| | CGGCATGCCAGTGTGTGAATTTGAT | | |
| | ATGGTTAAAGATCCAGAAGTACAGG | | |
| | ACTTCCGAAGAAATATTCTGAACGT | | |
| | TTGTAAAGAAGCTGTGGATC | | |
| 186573.9856677.PIK3CA.1_2 | TTAGGGACCTCAATTCACCTCATAG | 120 | 1017 |
| | TAGAGCAATGTATGTCTATCCTCCA | | |
| | AATGTAGAATCTTCACCAGAATTGC | | |
| | CAAAGCACATATATAATAAATTAGA | | |
| | TAAAGGTAAGAAAATGACTA | | |
| 186573_9856678_P1K3CA1_1 | CCCTTGAAAAATGAAAGAGAGATGG | 120 | 1018 |
| | TGATTGCATCTAATGTTTTCCTGTT | | |
| | ATAGGGCAAATAATAGTGGTGATCT | | |
| | GGGTAATAGTTTCTCCAAATAATGA | | |
| | CAAGCAGAAGTATACTCTGA | | |
| 186573_9856678_P1K3CA.1_2 | AAATCAACCATGACTGTGTACCACA | 120 | 1019 |
| | ACAAGTAATTGCTGAAGCAATCAGG | | |
| | AAAAAAACTCGAAGTATGTTGCTAT | | |
| | CCTCTGAACAACTAAAACTCTGTGT | | |
| | TTTAGAATATCAGGGCAAGT | | |
| 186573_9856678_PIK3CA.1_3 | ATATTTTAAAAGTGTGTGGATGTGA | 120 | 1020 |
| | TGAATACTTCCTAGAAAAATATCCT | | |
| | CTGAGTCAGTATAAGGTGAGTAACA | | |
| | AGTTTCAAAATATTAATTTTTAATT | | |
| | TAAAAAGTAATCACATTGAG | | |
| 186573.9856679.PIK3CA_1 | TTACTTTTAAAATGAAAAACCTTAC | 120 | 1021 |
| | AGGAAATGGCTCGCCCCCTTAATCT | | |
| | CTTACAGTATATAAGAAGCTGTATA | | |
| | ATGCTTGGGAGGATGCCCAATTTGA | | |
| | TGTTGATGGCTAAAGAAAGC | | |
| 185573_9856679_PIK3CA_2 | CTTTATTCTCAACTGCCAATGGACT | 120 | 1022 |
| | GTTTTACAATGCCATCTTATTCCAG | | |
| | ACGCATTTCCACAGCTACACCATAT | | |
| | ATGAATGGAGAAACATCTACAAAAT | | |
| | CCCTTTGGGTTATAAATAGT | | |
| 186573.9856679.PIK3CA_3 | GCACTCAGAATAAAAATTCTTTGTG | 120 | 1023 |
| | CAACCTACGTGAATGTAAATATTCG | | |
| | AGACATTGATAAGGTAAAGTCAAAT | | |
| | GCTGATGCTTATTATTTTATAGAAA | | |
| | TTATTTTAGATAACCTTTTT | | |
| 186573.9856680_P1K3CA.1_1 | CTACTTTTTTCTTTTAGATCTATGT | 120 | 1024 |
| | TCGAACAGGTATCTACCATGGAGGA | | |
| | GAACCCTTATGTGACAATGTGAACA | | |
| | CTCAAAGAGTACCTTGTTCCAATCC | | |
| | CAGGTAAGGAAGTATATAGA | | |
| 186573.9856681_PIK3CA.1_1 | TACATAGGTGGAATGAATGGCTGAA | 120 | 1025 |
| | TTATGATATATACATTCCTGATCTT | | |
| | CCTCGTGCTGCTCGACTTTGCCTTT | | |
| | CCATTTGCTCTGTTAAAGGCCGAAA | | |
| | GGGTGCTAAAGAGGTAAAGT | | |
| 186573.9856682.PIK3CA_1 | GTTTTATAATTTAGACTAGTGAATA | 120 | 1026 |
| | TTTTTCTTTGTTTTTTAAGGAACAC | | |
| | TGTCCATTGGCATGGGGAAATATAA | | |
| | ACTTGTTTGATTACACAGACACTCT | | |
| | AGTATCTGGAAAAATGGCTT | | |
| 186573_9856682.PIK3CA_2 | TGAATCTTTGGCCAGTACCTCATGG | 120 | 1027 |
| | ATTAGAAGATTTGCTGAACCCTATT | | |
| | GGTGTTACTGGATCAAATCCAAATA | | |
| | AAGTAAGGTTTTTATTGTCATAAAT | | |
| | TAGATATTTTTTATGGCAGT | | |
| 186573_9856683_PIK3CA_1 | AGTCAAACCTTCTCTCTTATGTATA | 120 | 1028 |
| | TATAATAGCTTTTCTTCCATCTCTT | | |
| | AGGAAACTCCATGCTTAGAGTTGGA | | |
| | GTTTGACTGGTTCAGCAGTGTGGTA | | |
| | AAGTTCCCAGATATGTCAGT | | |
| 186573_9856683.PIK3CA_2 | GATTGAAGAGCATGCCAATTGGTCT | 120 | 1029 |
| | GTATCCCGAGAAGCAGGATTTAGCT | | |
| | ATTCCCACGCAGGACTGGTAAGGCA | | |
| | AATCACTGAGTTTATTAAGTATCAA | | |
| | TTATAATCTGTGGATTTAGG | | |
| 186573.9856684.PIK3CA_1 | ATCCAGAGGGGAAAAATATGACAAA | 120 | 1030 |
| | GAAAGCTATATAAGATATTATTTTA | | |
| | TTTTACAGAGTAACAGACTAGCTAG | | |
| | AGACAATGAATTAAGGGAAAATGAC | | |
| | AAAGAACAGCTCAAAGCAAT | | |
| 186573.9856684.PIK3CA_2 | TTCTACACGAGATCCTCTCTCTGAA | 120 | 1031 |
| | ATCACTGAGCAGGAGAAAGATTTTC | | |
| | TATGGAGTCACAGGTAAGTGCTAAA | | |
| | ATGGAGATTCTCTGTTTCTTTTTCT | | |
| | TTATTACAGAAAAAATAACT | | |
| 186573.9856685.PIK3CA_1 | TTTTGTTTCTCCCACACAGACACTA | 120 | 1032 |
| | TTGTGTAACTATCCCCGAAATTCTA | | |
| | CCCAAATTGCTTCTGTCTGTTAAAT | | |
| | GGAATTCTAGAGATGAAGTAGCCCA | | |
| | GGTAAATGTATGTTTGAGAT | | |
| 186573_9856686_PIK3CA_1 | CCCCTTTAAATATGATTTATTGTCT | 120 | 1033 |
| | TTCTCATACACAGATGTATTGCTTG | | |
| | GTAAAAGATTGGCCTCCAATCAAAC | | |
| | CTGAACAGGCTATGGAACTTCTGGA | | |
| | CTGTAATTACCCAGATCCTA | | |
| 186573_9856686_PIK3CA_2 | TGGTTCGAGGTTTTGCTGTTCGGTG | 120 | 1034 |
| | CTTGGAAAAATATTTAACAGATGAC | | |
| | AAACTTTCTCAGTATTTAATTCAGC | | |
| | TAGTACAGGTAAAATAATGTAAAAT | | |
| | AGTAAATAATGTTTAATTAC | | |
| 186573_9856687_PIK3CA_1 | TCACCTAGGTCCTAAAATATGAACA | 120 | 1035 |
| | ATATTTGGATAACTTGCTTGTGAGA | | |
| | TTTTTACTGAAGAAAGCATTGACTA | | |
| | ATCAAAGGATTGGGCACTTTTTCTT | | |
| | TTGGCATTTAAAGTAAGTCT | | |
| 186573_9856688_PIK3CA_1_1 | TATATATATTTTTAATTTTGCACGA | 120 | 1036 |
| | TTCTTTTAGATCTGAGATGCACAAT | | |
| | AAAACAGTTAGCCAGAGGTTTGGCC | | |
| | TGCTTTTGGAGTCCTATTGTCGTGC | | |
| | ATGTGGGATGTATTTGAAGC | | |
| 186573_9856688_PIK3CA1_2 | ACCTGAATAGGCAAGTCGAGGCAAT | 120 | 1037 |
| | GGAAAAGCTCATTAACTTAACTGAC | | |
| | ATTCTCAAACAGGAGAAGAAGGATG | | |
| | AAACACAAAAGGTGTGTGACTCTAG | | |
| | TTTGTGTTTGAGACTCTTTT | | |
| 186573_9856689_PIK3CA_1 | AATCAGGTACAGATGAAGTTTTTAG | 120 | 1038 |
| | TTGAGCAAATGAGGCGACCAGATTT | | |
| | CATGGATGCTCTACAGGGCTTTCTG | | |
| | TCTCCTCTAAACCCTGCTCATCAAC | | |
| | TAGGAAACCTCAGGTACTTT | | |
| 186573_9856690_PIK3CA_1 | TTCAGGGTAAAATAATAATAAAGCA | 120 | 1039 |
| | AAGGTACCTAGTAAAGTTTTTAACT | | |
| | ATTTTAAAGGCTTGAAGAGTGTCGA | | |
| | ATTATGTCCTCTGCAAAAAGGCCAC | | |
| | TGTGGTTGAATTGGGAGAAC | | |
| 186573_9856690_PIK3CA_2 | CCAGACATCATGTCAGAGTTACTGT | 120 | 1040 |
| | TTCAGAACAATGAGATCATCTTTAA | | |
| | AAATGGGGATGGTAAGGAAGAGTAT | | |
| | TAATGAGCTTATGATGCATGAATTT | | |
| | AGCTATCTTTTTATACACAG | | |
| 186573_9856691_PIK3CA_1 | ACATATTATTTGAATTTCAGATTTA | 120 | 1041 |
| | CGGCAAGATATGCTAACACTTCAAA | | |
| | TTATTCGTATTATGGAAAATATCTG | | |
| | GCAAAATCAAGGTCTTGATCTTCGG | | |
| | TAGGTAACCAGTAAGGCAAC | | |
| 186573_9856692_P1K3CA_1 | TCATTTAATTGTAAACGTGTTACTC | 120 | 1042 |
| | CTCTTTCAGAATGTTACCTTATGGT | | |
| | TGTCTGTCAATCGGTGACTGTGTGG | | |
| | GACTTATTGAGGTGGTGCGAAATTC | | |
| | TCACACTATTATGCAAATTC | | |
| 186573_9856692_PIK3CA_2 | AGTGCAAAGGCGGCTTGAAAGGTGC | 120 | 1043 |
| | ACTGCAGTTCAACAGCCACACACTA | | |
| | CATCAGTGGCTCAAAGACAAGAACA | | |
| | AAGGAGAAATGTGAGTTGTATTATT | | |
| | CTTTCTTCCTATGTTAATCT | | |
| 186573_9856693_PIK3CA_1 | GATATGATGCAGCCATTGACCTGTT | 120 | 1044 |
| | TACACGTTCATGTGCTGGATACTGT | | |
| | GTAGCTACCTTCATTTTGGGAATTG | | |
| | GAGATCGTCACAATAGTAACATCAT | | |
| | GGTGAAAGACGATGGACAAG | | |
| 186573_9856694_PIK3CA.1_1 | ATCTGCATATATCAAACTATAACAT | 120 | 1045 |
| | AATTTCTTATTTTTGAAAGCTGTTT | | |
| | CATATAGATTTTGGACACTTTTTGG | | |
| | ATCACAAGAAGAAAAAATTTGGTTA | | |
| | TAAACGAGAACGTGTGCCAT | | |
| 185573_9856694_PIK3CA.1_2 | TTGTTTTGACACAGGATTTCTTAAT | 120 | 1046 |
| | AGTGATTAGTAAAGGAGCCCAAGAA | | |
| | TGCACAAAGACAAGAGAATTTGAGA | | |
| | GGTGAGCTCGAGCAATTAAAAACAC | | |
| | AAAATAAAGAGTTCTGGCTG | | |
| 186573_9856695_PIK3CA.1_1 | ACATCATTTGCTCCAAACTGACCAA | 120 | 1047 |
| | ACTGTTCTTATTACTTATAGGTTTC | | |
| | AGGAGATGTGTTACAAGGCTTATCT | | |
| | AGCTATTCGACAGCATGCCAATCTC | | |
| | TTCATAAATCTTTTCTCAAT | | |
| 186573_9856695_PIK3CA.1_2 | GATGCTTGGCTCTGGAATGCCAGAA | 120 | 1048 |
| | CTACAATCTTTTGATGACATTGCAT | | |
| | ACATTCGAAAGACCCTAGCCTTAGA | | |
| | TAAAACTGAGCAAGAGGCTTTGGAG | | |
| | TATTTCATGAAACAAATGAA | | |
| 186573_9856695_PIK3CA.1_3 | TGATGCACATCATGGTGGCTGGACA | 120 | 1049 |
| | ACAAAAATGGATTGGATCTTCCACA | | |
| | CAATTAAACAGCATGCATTGAACTG | | |
| | AAAAGATAACTGAGAAAATGAAAGC | | |
| | TCACTCTGGATTCCACACTG | | |
| 185573_9856696_FBXW7.1_1 | ATCTTACCTGTAATGAATAGACTCT | 120 | 1050 |
| | ATTAGTATGCCCCTGCAACGTGTGT | | |
| | AGACAGGTTTCAGTCTCTGGATCCC | | |
| | ACACCTTTACCATAAAATCATATGC | | |
| | TCCACTAACAACCCTCCTGC | | |
| 18S573_9856696_FBXW7.1_2 | CATCATATTGAACACAGCGGACTGC | 120 | 1051 |
| | TGCAACATGACCCATCAAAACATGT | | |
| | AAACACTGGCCTGTCTCAATATCCC | | |
| | AAACCCTAAGAGTGGCATCTCGAGA | | |
| | ACCGCTAACAACTCTGCAGA | | |
| 186573_9856697_TERT_1 | CTGCGCAGCAGGGAGCGCACGGCTC | 120 | 1052 |
| | GGCAGCGGGGAGCGCGCGGCATCGC | | |
| | GGGGGTGGCCGGGGCCAGGGCTTCC | | |
| | CACGTGCGCAGCAGGACGCAGCGCT | | |
| | GCCTGAAACTCGCGCCGCGA | | |
| 186573_9856697_TERT_2 | GGAGAGGGCGGGGCCGCGGAAAGGA | 120 | 1053 |
| | AGGGGAGGGGCTGGGAGGGCCCGGA | | |
| | GGGGGCTGGGCCGGGGACCCGGGAG | | |
| | GGGTCGGGACGGGGCGGGGTCCGCG | | |
| | CGGAGGAGGCGGAGCTGGAA | | |
| 186573_9856698_CDKN2A_1 | ACAAATTCTCAGATCATCAGTCCTC | 120 | 1054 |
| | ACCTGAGGGACCTTCCGCGGCATCT | | |
| | ATGCGGGCATGGTTACTGCCTCTGG | | |
| | TGCCCCCCGCAGCCGCGCGCAGGTA | | |
| | CCGTGCGACATCGCGATGGC | | |
| 186573_9856698_CDKN2A_2 | CCAGCTCCTCAGCCAGGTCCACGGG | 120 | 1055 |
| | CAGACGGCCCCAGGCATCGCGCACG | | |
| | TCCAGCCGCGCCCCGGCCCGGTGCA | | |
| | GCACCACCAGCGTGTCCAGGAAGCC | | |
| | CTCCCGGGCAGCGTCGTGCA | | |
| 186573_9856698_CDKN2A_3 | CGGGTCGGGTGAGAGTGGCGGGGTC | 120 | 1056 |
| | GGCGCAGTTGGGCTCCGCGCCGTGG | | |
| | AGCAGCAGCAGCTCCGCCACTCGGG | | |
| | CGCTGCCCATCATCATGACCTGCCA | | |
| | GAGAGAACAGAATGGTCAGA | | |
| 186573_9856699_CDKN2A_1 | GAGTCGCCCGCCATCCCCTGCTCCC | 120 | 1057 |
| | GCTGCAGACCCTCTACCCACCTGGA | | |
| | TCGGCCTCCGACCGTAACTATTCGG | | |
| | TGCGTTGGGCAGCGCCCCCGCCTCC | | |
| | AGCAGCGCCCGCACCTCCTC | | |
| 186573_9856699_CDKN2A_2 | TACCCGACCCCGGGCCGCGGCCGTG | 120 | 1058 |
| | GCCAGCCAGTCAGCCGAAGGCTCCA | | |
| | TGCTGCTCCCCGCCGCCGGCTCCAT | | |
| | GCTGCTCCCCGCCGCCCGCTGCCTG | | |
| | CTCTCCCCCTCTCCGCAGCC | | |
| 185573_9856700_CDKN2A_1 | GGACTTTTCGAGGGCCTTTCCTACC | 120 | 1059 |
| | TGGTCTTCTAGGAAGCGGCTGCTGC | | |
| | CCTAGACGCTGGCTCCTCAGTAGCA | | |
| | TCAGCACGAGGGCCACAGCGGCGGG | | |
| | CGCCCCTGGCGCTGCCCACT | | |
| 186573_9856700_CDKN2A_2 | CCCCCGTGAGCCGCGGGATGTGAAC | 120 | 1060 |
| | CACGAAAACCCTCACTCGCGGCGGG | | |
| | CCGCACGCGCGCCGAATCCGGAGGG | | |
| | TCACCAAGAACCTGCGCACCATGTT | | |
| | CTCGCCGCCTCCAGGGCCGA | | |
| 186573_9856701_NOTCH1_1 | GGTTTCCCGCCCTGGCCCCGGCCGA | 120 | 1061 |
| | CGCACCGGGCATGCAGATGCACTGG | | |
| | AACTCCCCAATCTGGTCCAGGCAGG | | |
| | TGGCGTCGTTCTGGCACGGGTTCGA | | |
| | GACGCACTCGTTGACGTCGA | | |
| 186573_9856701_NOTCH1_2 | TCTCGCATCGGGGGCCCGTGTAGCC | 120 | 1062 |
| | CTGCAGACACTGGCACTCGAAGGAG | | |
| | CCCAGCGTGTTGATGCACTTGCCCG | | |
| | CATGCTCGCAGGGGTTGGCACCTGG | | |
| | CGAGGGCACACGGGTGAGAG | | |
| 186573_9856702_NOTCH1_1 | GCGGCCATGGGGACACTCGCAGTAG | 120 | 1063 |
| | AAGGAGGCCACACGGTCATGGCAGG | | |
| | TGGCGCCGTGGAAGCAGGCGGCGCT | | |
| | GGCACAGTCATCAATGTTCTCGCTG | | |
| | CAGTCCTCACCAGTCCAGCC | | |
| 186573_9856702_NOTCH1_2 | GTTGACACACACGCAGTTGTAGCCA | 120 | 1064 |
| | CCGTGGGTGTTGTGGCAGGTCCCGC | | |
| | CGTTCTGGCAGGCATTTGGCATCAG | | |
| | CTGGCACTCGTCCACATCCTCGGTA | | |
| | CAGTACTGACCTGCAGGGAA | | |

1. A method for capturing circulating tumor DNA (ctDNA) of interest from an animal sample, preferably a mammalian sample, further preferably a human patient sample, comprising cell-free DNA (cfDNA), the method comprising:
adding to the patient sample a library of nucleic acid hybrid capture probes, wherein the library of probes is complementary to both strands of the double stranded ctDNA of interest and the probes are tagged for capture;
allowing the probes to hybridize to the ctDNA; and
capturing the hybridized ctDNA using the tag on the probes.

2. The method of claim 1, further comprising polymerase chain reaction (PCR) assembly to detect a specific ctDNA of interest.

3. The method of claim 1, further comprising sequencing the captured ctDNA.

4. The method of claim 3, wherein the sequencing comprises next-generation sequencing.

5. The method of claim 4, wherein the next-generation sequencing comprises Illumina, Roche 454, or Ion Torrent Sequencing.

6. The method of claim 1, wherein the probes complementary to one strand of the ctDNA are offset by 40-60% from the probes complementary to other strand.

7. The method of claim 6, wherein the probes complementary to one strand of the ctDNA are offset by 50% from the probes complementary to other strand.

8. The method of claim 1, wherein the probes are 50 bp to 160 bp in length.

9. The method of claim 8, wherein the probes are between 80 bp to 160 bp in length.

10. The method of claim 9, wherein the probes are between 100 bp and 140 bp in length.

11. The method of claim 10, wherein the probes are about 120 bp in length.

12. The method of claim 1, wherein the ctDNA of interest corresponds to a mutation of interest.

13. The method of claim 1, wherein the ctDNA of interest corresponds to a virus, preferably an oncogenic virus.

14. The method of claim 13, wherein the oncogenic virus is selected from the group consisting of human papillomavirus (HPV), Epstein-Barr virus (EBV), Kaposi's sarcoma-associated herpesvirus (KSHV), human T-lymphotropic virus (HTLV), or Merkle cell polyomavirus (MCV).

15. The method of claim 14, wherein the oncogenic virus is human papillomavirus (HPV).

16. The method of claim 1, wherein the library of probes covers substantially the entire genome of the oncogenic virus.

17. The method of claim 1, further comprising at least one of determining the fragment length of the ctDNA, genotyping the ctDNA, and mapping location of the ctDNA to the genome.

18. The method of claim 1, wherein the patient sample is selected from the group consisting of peripheral blood serum or plasma, urine, saliva, breast milk, cerebrospinal fluid, and synovial fluid.

19. The method of claim 18, wherein the patient sample is peripheral blood plasma.

20. The method of claim 1, for detecting cancer.

21. The method of claim 1, for monitoring cancer therapy.

22-32. (canceled)


SEQUENCE LISTING (1064 sequences)

SEQ ID NO: 1 - DNA, 120 bp, human papillomavirus
  gi|1020234.E6|lcl|HPV38_E6.1| Human papillomavirus 38 (HPV38), E6 gene_1
accctgtagattctgcaacagtttcctcacgtacattgaattacgtgagtttgattacaagaacttacagttaatctggactcaagaggattttgtttttgcatgttgtagcagttgtgc

SEQ ID NO: 2 - DNA, 120 bp, human papillomavirus
  gi|1020234.E6|lcl|HPV38_E6.1| Human papillomavirus 38 (HPV38), E6 gene_2
gcaacagacaataggccttattgttataaggtgtcagtattgtttaaagtgtcttgatttgatagaaaaattagatatctgttgctctcatcaagcatttcacaaggttagaggcaattg

SEQ ID NO: 3 - DNA, 120 bp, human papillomavirus
  gi|1020290.E6|lcl|HPV66_E6.1| Human papillomavirus 66 (HPV66), E6 gene_1
tttacttgatcttagattatcatgtgtatactgcaaaaaggaacttacaagtttagagctatataggtttgcatgtattgagttaaaactagtatatagaaacaattggccatatgcagt

SEQ ID NO: 4 - DNA, 120 bp, human papillomavirus
  gi|1020290.E6|lcl|HPV66_E6.1| Human papillomavirus 66 (HPV66), E6 gene_2
attagaaagtataactaaaaaacagttatctgatttatcaataaggtgctaccgatgtcaatgtccgttaacaccggaggaaaaacaattgcactgtgaacataaaagacgatttcatta

SEQ ID NO: 5 - DNA, 120 bp, human papillomavirus
  gi|1173493.E6|lcl|HPV70_E6.1| Human papillomavirus 70 (HPV70), E6 gene_1
gcacgacattacaatagactgtgtctattgtaaaacacagctacagcaaacagaggtatatgaatttgcatttagtgatttatttatagtatatagaaacggggagccatatgctgcatg

SEQ ID NO: 6 - DNA, 120 bp, human papillomavirus
  gi|1173493.E6|lcl|HPV70_E6.1| Human papillomavirus 70 (HPV70), E6 gene_2
ggaaagcataactaataccaagttatataatttatcaataaggtgcatgagttgcctgaaaccattgtgtccagcagaaaaattaaggcatgttaataccaaaagaagatttcaccaaat

SEQ ID NO: 7 - DNA, 120 bp, human papillomavirus
  gi|12084981.E6|lcl|HPV71_E6.1| Human papillomavirus 71 (HPV71), E6 gene_1
gtacgacgtggacctgcaggacctgaacctaacctgcatattctgcagaaatcaattaacagacgtggaagtcgtggcctttgcatataaggagctgaaagttgtgtggagaagtggttt

SEQ ID NO: 8 - DNA, 120 bp, human papillomavirus
  gi|12084981.E6|lcl|HPV71_E6.1| Human papillomavirus 71 (HPV71), E6 gene_2
ctttgcaaacacagtggaattagacaccggaacgccagttacagagcaactaatacggtgctacgtgtgtcacaagccattgtgtagtgtggaaaaagaaagaataattacagaaggcag

SEQ ID NO: 9 - DNA, 120 bp, human papillomavirus
  gi|12958167.E6|lcl|HPV84_E6.1| Human papillomavirus 84 (HPV84), E6 gene_1
acgattaatttgcatcttttgcaaggaagaattaacggaaggcgaagtgctggcctttgcagtaaaggaattactaattgtttggaggtataatttccctcatggggtgtgcatgaaatg

SEQ ID NO: 10 - DNA, 120 bp, human papillomavirus
  gi|12958167.E6|lcl|HPV84_E6.1| Human papillomavirus 84 (HPV84), E6 gene_2
aacaggattaccacttgcacaaataaatataaggtgccacgcgtgctgcaagccattgtgctatcaggaaaaggagtatatggtggaattgcagttgctattccacaaaatagctggaca

SEQ ID NO: 11 - DNA, 120 bp, human papillomavirus
  gi|1491683.E6|lcl|HPV72_E6.1| Human papillomavirus 72 (HPV72), E6 gene_1
acggattacctgcatattttgcaaaaatgaattaacaacagaagaattgctggcgattgcaataaaggagctgcagattgtgtggcgggacaactggccatttggagtctgcgcaccatg

SEQ ID NO: 12 - DNA, 120 bp, human papillomavirus
  gi|1491683.E6|lcl|HPV72_E6.1| Human papillomavirus 72 (HPV72), E6 gene_2
aacaggcaaatcattagcagaactatatataaggtgccatgcatgctgcaaacccctaagctgtcaggaaaaggaatatcaggtgcagacaggaatccacttccacaagataagcggact

SEQ ID NO: 13 - DNA, 120 bp, human papillomavirus
  gi|1491692.E6|lcl|HPV73_E6.1| Human papillomavirus 73 (HPV73), E6 gene_1
tgatataaacctggactgtgtgttttgccaacgtggactgtacagatctgaggtatatgattttgcatttagtgatttgtgtattgtatatagaaaggataaaccatatggtgtatgtca

SEQ ID NO: 14 - DNA, 120 bp, human papillomavirus
  gi|1491692.E6|lcl|HPV73_E6.1| Human papillomavirus 73 (HPV73), E6 gene_2
aaatttaactaacaaacagttatgtaatattttaataaggtgcggaaaatgccaaaaaccattatgtccactggaaaagcaaaagcatgtagatgaaaaaaaacggtttcatcaaatagc

SEQ ID NO: 15 - DNA, 120 bp, human papillomavirus
  gi|222386.E6|lcl|HPV58_E6.1| Human papillomavirus 58 (HPV58), E6 gene_1
aatcgaattgaaatgcgttgaatgcaaaaagactttgcagcgatctgaggtatatgactttgtatttgcagatttaagaatagtgtatagagatggaaatccatttgcagtatgtaaagt

SEQ ID NO: 16 - DNA, 120 bp, human papillomavirus
  gi|222386.E6|lcl|HPV58_E6.1| Human papillomavirus 58 (HPV58), E6 gene_2
aacactaaaaaagtgtttaaatgaaatattaattagatgtattatttgtcaaagaccattgtgtccacaagaaaaaaaaaggcatgtggatttaaacaaaaggtttcataatatttcggg

SEQ ID NO: 17 - DNA, 120 bp, human papillomavirus
  gi|3228267.E6|lcl|HPV67_E6.1| Human papillomavirus 67 (HPV67), E6 gene_1
aataagtttgccctgcgtgcagtgcaaaaaaactttggacagaaacgaggtatatgactttttatttactgatttgaaaatagtatacagatgtgggaatccgtatggggtatgtaagca

SEQ ID NO: 18 - DNA, 120 bp, human papillomavirus
  gi|3228267.E6|lcl|HPV67_E6.1| Human papillomavirus 67 (HPV67), E6 gene_2
aattgtgcataaaccattaaatgaaattacaatacgatgtataacatgtcaaagaccattgtgtccacaagaaaagcaaaggcatgttgatcgcaaaaagcgatttcataatatttccaa

SEQ ID NO: 19 - DNA, 120 bp, human papillomavirus
  gi|333026.E6|lcl|HPV11_E6.1| Human papillomavirus 11 (HPV11), E6 gene_1
cactctgcaaattcagtgcgtgttttgcaggaatgcactgaccaccgcagagatatatgcatatgcctataagaacctaaaggttgtgtggcgagacaactttccctttgcagcgtgtgc

SEQ ID NO: 20 - DNA, 120 bp, human papillomavirus
  gi|333026.E6|lcl|HPV11_E6.1| Human papillomavirus 11 (HPV11), E6 gene_2
agaagaaaccaatgaagatattttaaaagtgttaattcgttgttacctgtgtcacaagccgttgtgtgaaatagaaaaactaaagcacatattgggaaaggcacgcttcataaaactaaa

SEQ ID NO: 21 - DNA, 120 bp, human papillomavirus
  gi|333031.E6|lcl|HPV16_E6.1| Human papillomavirus 16 (HPV16), E6 gene_1
gctgcaaacaactatacatgatataatattagaatgtgtgtactgcaagcaacagttactgcgacgtgaggtatatgactttgcttttcgggatttatgcatagtatatagagatgggaa

SEQ ID NO: 22 - DNA, 120 bp, human papillomavirus
  gi|333031.E6|lcl|HPV16_E6.1| Human papillomavirus 16 (HPV16), E6 gene_2
gtatggaacaacattagaacagcaatacaacaaaccgttgtgtgatttgttaattaggtgtattaactgtcaaaagccactgtgtcctgaagaaaagcaaagacatctggacaaaaagca

SEQ ID NO: 23 - DNA, 120 bp, human papillomavirus
  gi|333048.E6|lcl|HPV31_E6.1| Human papillomavirus 31 (HPV31), E6 gene_1
actaagattgaattgtgtctactgcaaaggtcagttaacagaaacagaggtattagattttgcatttacagatttaacaatagtatatagggacgacacaccacacggagtgtgtacaaa

SEQ ID NO: 24 - DNA, 120 bp, human papillomavirus
  gi|333048.E6|lcl|HPV31_E6.1| Human papillomavirus 31 (HPV31), E6 gene_2
attgacaaacaaaggtatatgtgatttgttaattaggtgtataacgtgtcaaagaccgttgtgtccagaagaaaaacaaagacatttggataaaaagaaacgattccacaacataggagg

SEQ ID NO: 25 - DNA, 120 bp, human papillomavirus
  gi|333049.E6|lcl|HPV33_E6.1| Human papillomavirus 33 (HPV33), E6 gene_1
cattgaactacagtgcgtggaatgcaaaaaacctttgcaacgatctgaggtatatgattttgcatttgcagatttaacagttgtatatagagagggaaatccatttggaatatgtaaact

SEQ ID NO: 26 - DNA, 120 bp, human papillomavirus
  gi|333049.E6|lcl|HPV33_E6.1| Human papillomavirus 33 (HPV33), E6 gene_2
aacagttaaaaaacctttaaatgaaatattaattaggtgtattatatgtcaaagacctttgtgtcctcaagaaaaaaaacgacatgtggatttaaacaaacgatttcataatatttcggg

SEQ ID NO: 27 - DNA, 120 bp, human papillomavirus
  gi|333071.E6|lcl|HPV5_E6.1| Human papillomavirus 5 (HPV5), E6 gene_1
cttagctgaagccttaggcatccctgtgattgattgtttaataccttgcaatttctgtggcaactttctaaattatttggaagcttgtgaattcgactacaaaaggcttagtctaatttg

SEQ ID NO: 28 - DNA, 120 bp, human papillomavirus
  gi|333071.E6|lcl|HPV5_E6.1| Human papillomavirus 5 (HPV5), E6 gene_2
tgagcagacagtgttaggaagagatattgaattagcttcaggactttcaatatttgatattgatatcaggtgtcaaacttgcttagcatttcttgacattatagaaaagttagattgctg

SEQ ID NO: 29 - DNA, 120 bp, human papillomavirus
  gi|333087.E6|lcl|HPV51_E6.1| Human papillomavirus 51 (HPV51), E6 gene_1
tatacaggtagtgtgtgtgtattgtaaaaaggaattatgtagagcagatgtatataatgtagcatttactgaaattaagattgtatatagggataataatccatatgcagtatgcaaaca

SEQ ID NO: 30 - DNA, 120 bp, human papillomavirus
  gi|333087.E6|lcl|HPV51_E6.1| Human papillomavirus 51 (HPV51), E6 gene_2
aattactaaaaaaagcttatatgatttatcgataaggtgtcatagatgtcaaagaccacttgggcctgaagaaaagcaaaaattggtggacgaaaaaaaaaggttccatgaaatagcggg

SEQ ID NO: 31 - DNA, 120 bp, human papillomavirus
  gi|333211.E6|lcl|HPV42_E6.1| Human papillomavirus 42 (HPV42), E6 gene_1
gaatttacagatttcctgcatttggtgcaaaaagcacttaacaggcgcagaggtgctcgcgtaccattttaaagatttggtagtggtgtggaggaaggactttccatatgctgcatgtgc

SEQ ID NO: 32 - DNA, 120 bp, human papillomavirus
  gi|333211.E6|lcl|HPV42_E6.1| Human papillomavirus 42 (HPV42), E6 gene_2
gaaagaaactggactacttttagaagaacaacaaattagatgtgccttgtgtcaaaagccgttatcacagagcgaaaaaaaccatcatattgatacaggtacaagatttcaatttatatt

SEQ ID NO: 33 - DNA, 120 bp, human papillomavirus
  gi|333245.E6|lcl|HPV39_E6.1| Human papillomavirus 39 (HPV39), E6 gene_1
gcaggacattacaatagcctgtgtctattgcagacgaccactacagcaaaccgaggtatatgaatttgcatttagtgatttatatgtagtatatagggacggggaaccactagctgcatg

SEQ ID NO: 34 - DNA, 120 bp, human papillomavirus
  gi|333245.E6|lcl|HPV39_E6.1| Human papillomavirus 39 (HPV39), E6 gene_2
agaaaatataactaatacaaagttatataatttattaataaggtgcatgtgttgtctgaaaccgctgtgtccagcagaaaaattaagacacctaaatagcaaacgaagatttcataaaat

SEQ ID NO: 35 - DNA, 120 bp, human papillomavirus
  gi|396910.E6|lcl|HPV12_E6.1| Human papillomavirus 12 (HPV12), E6 gene_1
tgaccttttagatatacctttagttgactgtttggtaccttgcaatttttgcggaaagttcttagattttctggaagtgtgtgattttgacaaaaagcagctaacactaatttggaaagg

SEQ ID NO: 36 - DNA, 120 bp, human papillomavirus
  gi|396910.E6|lcl|HPV12_E6.1| Human papillomavirus 12 (HPV12), E6 gene_2
aacagtgctaggtagagatatagagcttgctactggaaaatctatatttgacttaaagataaggtgtcagacgtgcttgtcatttttagatacaattgaaaagttagacagctgtggtcg

SEQ ID NO: 37 - DNA, 120 bp, human papillomavirus
  gi|396937660|emb|HE963220.1| Human papillomavirus type 55 (HPV55) E6 gene, isolate CAC1126-10_1
ccatttgcaaattttatgcgtgttttgcagaaaaaccttgaccactgcagaggtttattcattcgcatataaacagttatatgtagtgtttcgaggtaactttccatttgcagcctgtgc

SEQ ID NO: 38 - DNA, 120 bp, human papillomavirus
  gi|396937660|emb|HE963220.1| Human papillomavirus type 55 (HPV55) E6 gene, isolate CAC1126-10_2
agaagagacaaagcagtcaattttggacgtgttcatacgctgttatttgtgccacaaaccattgtgccacgtggaaaaggtgcgccacatattggagaaggcgcgatttattaaattaca

SEQ ID NO: 39 - DNA, 120 bp, human papillomavirus
  gi|396997.E6|lcl|HPV35_E6.1| Human papillomavirus 35 (HPV35), E6 gene_1
aatttgtttgaattgtgtatactgcaaacaagaattacagcggagtgaggtatatgactttgcatgctatgatttgtgtatagtatatagagaaggccagccatatggagtatgcatgaa

SEQ ID NO: 40 - DNA, 120 bp, human papillomavirus
  gi|396997.E6|lcl|HPV35_E6.1| Human papillomavirus 35 (HPV35), E6 gene_2
acaatgcaacaaacagttatgtcatttattaattaggtgtattacatgtcaaaaaccgctgtgtccagttgaaaagcaaagacatttagaagaaaaaaaacgattccataacatcggtgg

SEQ ID NO: 41 - DNA, 120 bp, human papillomavirus
  gi|397014.E6|lcl|HPV40_E6.1| Human papillomavirus 40 (HPV40), E6 gene_1
gttgcaaattgattgtgtgttttgcaagacggtcctaaaaacagctgaggtactggcctttgcctttagagagttatatgttgtgtggcgcgacgactttccacacgccgcatgtccacg

SEQ ID NO: 42 - DNA, 120 bp, human papillomavirus
  gi|397014.E6|lcl|HPV40_E6.1| Human papillomavirus 40 (HPV40), E6 gene_2
agagacaggattaaccattttacaagtaaggattagatgctgcaagtgccacaagcctttgtctcccgtggaaaaaaccaaccatattgtaaagaagacgcaattctttaaattaaaaga

SEQ ID NO: 43 - DNA, 120 bp, human papillomavirus
  gi|397022.E6|lcl|HPV45_E6.1| Human papillomavirus 45 (HPV45), E6 gene_1
acaagacgtatctattgcctgtgtatattgcaaagcaacattggaacgcacagaggtatatcaatttgcttttaaagatttatgtatagtgtatagagactgtatagcatatgctgcatg

SEQ ID NO: 44 - DNA, 120 bp, human papillomavirus
  gi|397022.E6|lcl|HPV45_E6.1| Human papillomavirus 45 (HPV45), E6 gene_2
ggaaaaaataactaatacagagttgtataatttgttaataaggtgcctgcggtgccagaaaccattgaacccagcagaaaaacgtagacaccttaaggacaaacgaagatttcacagcat

SEQ ID NO: 45 - DNA, 120 bp, human papillomavirus
  gi|397038.E6|lcl|HPV52_E6.1| Human papillomavirus 52 (HPV52), E6 gene_1
aataaggctgcagtgtgtgcagtgcaaaaaagagctacaacgaagagaggtatacaagtttctatttacagatttacgaatagtatatagagacaataatccatatggcgtgtgtattat

SEQ ID NO: 46 - DNA, 120 bp, human papillomavirus
  gi|397038.E6|lcl|HPV52_E6.1| Human papillomavirus 52 (HPV52), E6 gene_2
gagggtaaaaaaaccattaagtgaaataactattagatgtataatttgtcaaacgccattatgtcctgaagaaaaagaaagacatgttaatgcaaacaagcgatttcataatattatggg

SEQ ID NO: 47 - DNA, 120 bp, human papillomavirus
  gi|397053.E6|lcl|HPV56_E6.1| Human papillomavirus 56 (HPV56), E6 gene_1
tttaattgatcttagattatcatgtgtatattgcaaaaaagaactaacacgtgctgaggtatataattttgcatgcactgaattaaaattagtgtatagggatgattttccttatgcagt

SEQ ID NO: 48 - DNA, 120 bp, human papillomavirus
  gi|397053.E6|lcl|HPV56_E6.1| Human papillomavirus 56 (HPV56), E6 gene_2
actagaaagtataactaaaaaacagttatgtgatttattaataaggtgctacagatgtcaaagtccgttaactccggaggaaaagcaattgcattgtgacagaaaaagacgatttcatct

SEQ ID NO: 49 - DNA, 120 bp, human papillomavirus
  gi|39932599.E6|lcl|HPV62_E6.1| Human papillomavirus 62 (HPV62), E6 gene_1
gcacctaacctgcatattttgcaaaacagacctgtccgccggtgaactgctgtcctttgcaatacgggagctacacgtggtgcggcgaaaggactggccatttggagtatgcgcagcatg

SEQ ID NO: 50 - DNA, 120 bp, human papillomavirus
  gi|39932599.E6|lcl|HPV62_E6.1| Human papillomavirus 62 (HPV62), E6 gene_2
aacaggcagtccgcttgcacaaatatatattagatgccatgcatgctgtatgccattaacctgtcaggaaaaggagtatcaggtagagaggggagtgcacttccacaaaatagcaggagt

SEQ ID NO: 51 - DNA, 120 bp, human papillomavirus
  gi|40804509.E6|lcl|HPV81_E6.1| Human papillomavirus 81 (HPV81), E6 gene_1
agtggacctagacgacctgcaactgacctgcatattttgcaaaaaggaattaacagtaggtgaattgctgtcctttgcaataagagagctaaacttggtgtggcgaaagaactggccatt

SEQ ID NO: 52 - DNA, 120 bp, human papillomavirus
  gi|40804509.E6|lcl|HPV81_E6.1| Human papillomavirus 81 (HPV81), E6 gene_2
accaacagtagaagaggagacagggcaaccgcttgcacaaatatatatacgatgccatgcatgctataaaccgttaacataccaagaaaaggagtatcttgtgacaggcctaatccactt

SEQ ID NO: 53 - DNA, 120 bp, human papillomavirus
  gi|50253426.E6|lcl|HPV96_E6.1| Human papillomavirus 96 (HPV96), E6 gene_1
cttctgtaagaaatttttaacctttgaggagctattaggatttgatagcaagactttaaatcttatttggaaggaaggttatgcatatgcgtgttgtttcaattgtgctagaacagtagc

SEQ ID NO: 54 - DNA, 120 bp, human papillomavirus
  gi|50253426.E6|lcl|HPV96_E6.1| Human papillomavirus 96 (HPV96), E6 gene_2
ttgttgtattgttgttagatgccagttttgtttgcgactgttagattatttagaaaagttaaatatttgtgggtatcaagaaaacttttatttagtaagaggaggttggaaaggcatttg

SEQ ID NO: 55 - DNA, 120 bp, human papillomavirus
  gi|5059324.E6|lcl|HPV83_E6.1| Human papillomavirus 83 (HPV83), E6 gene_1
actgatttgcatttattgcacaaatgaattaacaacagcagaagtgctgtcctttgcatggaaggagctgtgtattaagtgggaccacgaactgccctacggagcgtgtgcacagtgtct

SEQ ID NO: 56 - DNA, 120 bp, human papillomavirus
  gi|5059324.E6|lcl|HPV83_E6.1| Human papillomavirus 83 (HPV83), E6 gene_2
aaaaacaccactggcacagctgtatatacgatgccatatgtgcctgaagccgttgagtagtcaagaaaaagaatatttggtgcaaacaggtgatagacttcataatatagcaggacagtg

SEQ ID NO: 57 - DNA, 120 bp, human papillomavirus
  gi|557236.E6|lcl|HPV59_E6.1| Human papillomavirus 59 (HPV59), E6 gene_1
gcatgatattcgcatcaattgtgtgttttgcaaaggggaactgcaagaaagagaggtatttgaatttgcttttaatgacttatttatagtgtatagagactgtacaccgtatgcagcgtg

SEQ ID NO: 58 - DNA, 120 bp, human papillomavirus
  gi|557236.E6|lcl|HPV59_E6.1| Human papillomavirus 59 (HPV59), E6 gene_2
agaggctgaaaccaagacaccgttacatgagctgctgatacgctgttatagatgcctaaaacctctatgtccaacagataaattaaagcatataactgaaaaaagaagattccataatat

SEQ ID NO: 59 - DNA, 120 bp, human papillomavirus
  gi|60955.E6|lcl|HPV6_E6.1| Human papillomavirus 6 (HPV6), E6 gene_1
tacgttgcaaattaattgtgtgttttgcaagaatgcactgaccacagcagagatttattcatatgcatataaacacctaaaggtcctgtttcgaggcggctatccatatgcagcctgcgc

SEQ ID NO: 60 - DNA, 120 bp, human papillomavirus
  gi|60955.E6|lcl|HPV6_E6.1| Human papillomavirus 6 (HPV6), E6 gene_2
agaagaaactaaacaagacatcttagacgtgctaattcggtgctacctgtgtcacaaaccgctgtgtgaagtagaaaaggtaaaacatatactaaccaaggcgcggttcataaagctaaa

SEQ ID NO: 61 - DNA, 120 bp, human papillomavirus
  gi|60975.E6|lcl|HPV18_E6.1| Human papillomavirus 18 (HPV18), E6 gene_1
gcaagacatagaaataacctgtgtatattgcaagacagtattggaacttacagaggtatttgaatttgcatttaaagatttatttgtggtgtatagagacagtataccgcatgctgcatg

SEQ ID NO: 62 - DNA, 120 bp, human papillomavirus
  gi|60975.E6|lcl|HPV18_E6.1| Human papillomavirus 18 (HPV18), E6 gene_2
ggaaaaactaactaacactgggttatacaatttattaataaggtgcctgcggtgccagaaaccgttgaatccagcagaaaaacttagacaccttaatgaaaaacgacgatttcacaacat

SEQ ID NO: 63 - DNA, 120 bp, human papillomavirus
  gi|6970418.E6|lcl|HPV69_E6.1| Human papillomavirus 69 (HPV69), E6 gene_1
tttgcaggtacagtgtgtatattgcaagaaaacattagaatgggcagatgtatataactttgcaatatgtgatttaagaatagtgtatagaaatgatagtgcatatggtgcatgtaaaaa

SEQ ID NO: 64 - DNA, 120 bp, human papillomavirus
  gi|6970418.E6|lcl|HPV69_E6.1| Human papillomavirus 69 (HPV69), E6 gene_2
gcgtcctaaacgaagtttgtgtaatttgttaataaggtgtcatagatgccaaataccattgggaccggaagaaaaacagagaattgtggatgaaaagcgacggttccatgaaatagcagg

SEQ ID NO: 65 - DNA, 120 bp, human papillomavirus
  gi|6970427.E6|lcl|HPV82_E6.1| Human papillomavirus 82 (HPV82), E6 gene_1
tattcaggtattgtgtgtatattgtaaaaaggagttgtgtagagcagatgtgtataatgtagcatttacagaacttaggattgtatatagggacaatacgccatatgcagcatgcaaaaa

SEQ ID NO: 66 - DNA, 120 bp, human papillomavirus
  gi|6970427.E6|lcl|HPV82_E6.1| Human papillomavirus 82 (HPV82), E6 gene_2
cattactaacaaaagtttatatgaattattaataaggtgtcatagatgtcagagaccacttgggcctgaagaaaagcaaaaggtggtggacgacaaaaaaaggtttcatgaaatagcggg

SEQ ID NO: 67 - DNA, 120 bp, human papillomavirus
  gi|71726685.E6|lcl|HPV68_E6.1| Human papillomavirus 68 (HPV68), E6 gene_1
gcatgacgttacaatagactgtgtctattgcagaaggcaactacaacggacagaggtatatgaatttgcctttagtgacctatgtgtagtgtatagagacggggtaccatttgctgcatg

SEQ ID NO: 68 - DNA, 120 bp, human papillomavirus
  gi|71726685.E6|lcl|HPV68_E6.1| Human papillomavirus 68 (HPV68), E6 gene_2
agaaaccataactaatacaaagttatataatttattgataaggtgcatgagttgcctgaaaccattgtgtccagcagaaaaactaaggcacctaacaacaaaacgaagattacataaaat

SEQ ID NO: 69 - DNA, 120 bp, human papillomavirus
  gi|9627305.E6|lcl|HPV26_E6.1| Human papillomavirus 26 (HPV26), E6 gene_1
tttgcaggtacagtgtgtatattgcaaggaaaccttacaatgggctgatgtatataattttgcaatttgtgacctaagagtagtatatagagataggagtccgtatgctgcatgcaaaag

SEQ ID NO: 70 - DNA, 120 bp, human papillomavirus
  gi|9627305.E6|lcl|HPV26_E6.1| Human papillomavirus 26 (HPV26), E6 gene_2
cttaactaaaaaaagtttatgtaatttgttaataaggtgtcatagatgtcaaatgccattggggccagaagaaaaacaaagaattgtggatgaaaagcgacgatttcacgaaatagcagg

SEQ ID NO: 71 - DNA, 120 bp, human papillomavirus
  gi|9627377.E6|lcl|HPV53_E6.1| Human papillomavirus 53 (HPV53), E6 gene_1
accattgctggagctgcaacttggctgtgtgttctgcaagaaggcattgacagcgtcagaggtatataattttgcatatacagatctaagagtagtgtatagagacgggtatccgtatgg

SEQ ID NO: 72 - DNA, 120 bp, human papillomavirus
  gi|9627377.E6|lcl|HPV53_E6.1| Human papillomavirus 53 (HPV53), E6 gene_2
tagcctggaagcactaactaaaaaaaagttatctgatttatcaataaggtgctacagatgtcaacatccgttgacaccagaggaaaaacagttacactgtgactataagaaacggtttca

SEQ ID NO: 73 - DNA, 120 bp, human papillomavirus
  gi|9628437.E6|lcl|HPV54_E6.1| Human papillomavirus 54 (HPV54), E6 gene_1
cagttatttgggggcaatgtctgctactgaaccccacacggaccagccgcgtactctagctgatttgtgcaaggtatgcaatattcctatgcatagtttgcaacttccttgtgccttttg

SEQ ID NO: 74 - DNA, 120 bp, human papillomavirus
  gi|9628437.E6|lcl|HPV54_E6.1| Human papillomavirus 54 (HPV54), E6 gene_2
tccacatgctgcatgtgcactgtgcctagaactgcacgggcaaataaattatagaaggcatcgcgaccgtgcgtgcctgtgggaaacagtggaacaagagtgtggaaagccattggaaga

SEQ ID NO: 75 - DNA, 120 bp, human papillomavirus
  gi|9628574.E6|lcl|HPV61_E6.1| Human papillomavirus 61 (HPV61), E6 gene_1
gacatgtgtattttgcaaaaatgaattaacaacagaagaattgctggcgtttgcactaaaggagctaagcattgtgtggagacataactggccatttggagtatgcgcaccatgcttggc

SEQ ID NO: 76 - DNA, 120 bp, human papillomavirus
  gi|9628574.E6|lcl|HPV61_E6.1| Human papillomavirus 61 (HPV61), E6 gene_2
acggtcattagctgaactatatatacggtgccatgcatgcagcaaaccgttaagtatacaggaaaaggagcatcaggtacaggcatacatccacttccactatatagctggacagtggac

SEQ ID NO: 77 - DNA, 120 bp, human papillomavirus
  133448_5511683_gi|333071.E7|lcl|HPV5_E7.1|Humanpapillomavirus5_1_2
ttaccaaacgagcaggaaacggaggaggagcctgacaacgaaaggatctcttacaaagttatagctccgtgcggttgcaggaactgtgaggtcaagcttcgcatttttgtccacgccaca

SEQ ID NO: 78 - DNA, 120 bp, human papillomavirus
  133448_5511684_gi|60955.E7|lcl|HPV6_E7.1|Humanpapillomavirus6_1_2
tagacagctcagaagatgaggtggacgaagtggacggacaagattcacaacctttaaaacaacatttccaaatagtgacctgttgctgtggatgtgacagcaacgttcgactggttgtgc

SEQ ID NO: 79 - DNA, 120 bp, human papillomavirus
  133448_5511685_gi|333026.E7|lcl|HPV11_E7.1|Humanpapillomavirus_1_2
aagacagctcagaagatgaggtggacaaggtggacaaacaagacgcacaacctttaacacaacattaccaaatactgacctgttgctgtggatgtgacagcaacgtccgactggttgtgg

SEQ ID NO: 80 - DNA, 120 bp, human papillomavirus
  133448_5511686_gi|396910.E7|lcl|HPV12_E7.1|Humanpapillomavirus_1_2
ttaccaaacgagcaggaaacggaggaggagtcagatatcgacaggactgtattcaaaatcattgcaccgtgtggctgcagctcctgtgaggtcaaccttcgtatttttgtcaacgcaact

SEQ ID NO: 81 - DNA, 120 bp, human papillomavirus
  133448_5511687_gi|333031.E7|lcl|HPV16_E7.1|Humanpapillomavirus_1_2
acagctcagaggaggaggatgaaatagatggtccagctggacaagcagaaccggacagagcccattacaatattgtaaccttttgttgcaagtgtgactctacgcttcggttgtgcgtac

SEQ ID NO: 82 - DNA, 120 bp, human papillomavirus
  133448_5511688_gi|60975.E7|lcl|HPV18_E7.1|Humanpapillomavirus1_1_2
tcagaggaagaaaacgatgaaatagatggagttaatcatcaacatttaccagcccgacgagccgaaccacaacgtcacacaatgttgtgtatgtgttgtaagtgtgaagccagaattgag

SEQ ID NO: 83 - DNA, 120 bp, human papillomavirus
  133448_5511689_gi|9627305.E7|lcl|HPV26_E7.1|Humanpapillomavirus_1_ 2
atttgacagctcagatgaggatgaaacagataatatgcgtgaccagcaggccagacaagctggacaagaagtgtgttacagaattgaagcacaatgttgtatgtgtaatagtatagtgca

SEQ ID NO: 84 - DNA, 120 bp, human papillomavirus
  133448_5511690_gi|333048.E7|lcl|HPV31_E7.1|Humanpapillomavirus_1_2
acagctcagatgaggaggatgtcatagacagtccagctggacaagcagaaccggacacatccaattacaatatcgttaccttttgttgtcagtgtaagtctacacttcgtttgtgtgtac

SEQ ID NO: 85 - DNA, 120 bp, human papillomavirus
  133448_5511691_gi|333049.E7|lcl|HPV33_E7.1|Humanpapillomavirus_1_2
gacagctcagatgaggatgaaggcttggaccggccagatggacaagcacaaccagccacagctgattactacattgtaacctgttgtcacacttgtaacaccacagttcgtttatgtgtc

SEQ ID NO: 86 - DNA, 120 bp, human papillomavirus
  133448_5511692_gi|396997.E7|lcl|HPV35_E7.1|Humanpapillomavirus_1_2
agctcagaggaggaggaagatactattgacggtccagctggacaagcaaaaccagacacctccaattataatattgtaacgtcctgttgtaaatgtgaggcgacactacgtctgtgtgta

SEQ ID NO: 87 - DNA, 120 bp, human papillomavirus
  133448_5511693_gi|1020234.E7|lcl|HPV38_E7.1|Humanpapillomavirus_1_ 2
tccagaggatattgaagcatcagtggtagaggaggagccagcatacaccccatacaaaatcatagttctttgtgggggttgtgaagtaaggctaaaactatacgtgtgggccaccgacgc

SEQ ID NO: 88 - DNA, 120 bp, human papillomavirus
  133448_5511694_gi|333245.E7|lcl|HPV39_E7.1|Humanpapillomavirus_1_2
gaggatgaaatagatgaacccgaccatgcagttaatcaccaacatcaactactagccagacgggatgaaccacagcgtcacacaatacagtgttcgtgttgtaagtgtaacaacacactg

SEQ ID NO: 89 - DNA, 120 bp, human papillomavirus
  133448_5511695_gi|397014.E7|lcl|HPV40_E7.1|Humanpapillomavirus_1_2
catgaacaggaccaactagacagcttacacagtagagagcgtgagcaacccacgcaacaggacctgcaagtaaatttgcaatcatttaaagtagtaactcggtgtgtattttgtcagtgt

SEQ ID NO: 90 - DNA, 120 bp, human papillomavirus
  133448_5511696_gi|333211.E7|lcl|HPV42_E7.1|Humanpapillomavirus_1_2
tatgaacaattggacagctcagatgaagatgaccaagccaaacaggacatacagcgttacagaatactgtgtgtgtgtacacagtgttacaagtctgttaaactcgttgtgcagtgtaca

SEQ ID NO: 91 - DNA, 120 bp, human papillomavirus
  133448_5511697_gi|397022.E7|lcl|HPV45_E7.1|Humanpapillomavirus_1_2
agtcagaggaggaaaacgatgaagcagatggagttagtcatgcacaactaccagcccgacgagccgaaccacagcgtcacaaaattttgtgtgtatgttgtaagtgtgacggcagaattg

SEQ ID NO: 92 - DNA, 120 bp, human papillomavirus
  133448_5511698_gi|333087.E7|lcl|HPV51_E7.1|Humanpapillomavirus_1_2
tcagaggaggaggatgaagtagataatatgcgtgaccagctaccagaaagacgggctggacaggctacgtgttacagaattgaagctccgtgttgcaggtgttcaagtgtagtacaactg

SEQ ID NO: 93 - DNA, 120 bp, human papillomavirus
  133448_5511699_gi|397038.E7|lcl|HPV52_E7.1|Humanpapillomavirus_1_2
agctcagatgaggaggatacagatggtgtggaccggccagatggacaagcagaacaagccacaagcaattactacattgtgacatattgtcacagttgtgatagcacactacggctatgc

SEQ ID NO: 94 - DNA, 120 bp, human papillomavirus
  133448_5511700_gi|9627377.E7|lcl|HPV53_E7.1|Humanpapillomavirus_1_ 2
gatgaggatgaggatgaagtagaccatctgcaggagcagccacagcaagctagacgggacgaacaacatccttgttacctaattgaaacacagtgttgtaggtgtgagtcgttggtgcag

SEQ ID NO: 95 - DNA, 120 bp, human papillomavirus
  133448_5511701_gi|9628437.E7|lcl|HPV54_E7.1|Humanpapillomavirus_1_ 2
gaagactctgatgcagaagatgagacagcagtaacacaacctgacaaacaagcatttaaggtgttaagccagtgtggaggtgtgtgttgtaagacagtacggctatgtgtgtatagcacg

SEQ ID NO: 96 - DNA, 120 bp, human papillomavirus
  133448_5511702_gb|U31791.1|HPU31791530-823Humanpapillomavirus_1_2
gacagctcagaggatgaggtggatgaactagccacgcaggccacgcaagacgttacacagccttaccaaatagtaaccacctgtggtacatgtaaccggaacgttcggctggttgtgcag

SEQ ID NO: 97 - DNA, 120 bp, human papillomavirus
  133448_5511703_gi|397053.E7|lcl|HPV56_E7.1|Humanpapillomavirus_1_2
gatgaggatgaggatgaagtagaccatttgcaggagcggccacagcaagctagacaagctaaacaacatacgtgttacctaatacacgtaccttgttgtgagtgtaagtttgtggtgcag

SEQ ID NO: 98 - DNA, 120 bp, human papillomavirus
  133448_5511704_gi|222386.E7|lcl|HPV58_E7.1|Humanpapillomavirus_1_2
acagctcagacgaggatgaaataggcttggacgggccagatggacaagcacaaccggccacagctaattactacattgtaacttgttgttacacttgtggcaccacggttcgtttgtgta

SEQ ID NO: 99 - DNA, 120 bp, human papillomavirus
  133448_5511705_gi|557236.E7|lcl|HPV59_E7.1|Humanpapillomavirus_1_2
gactccgagaatgaaaaagatgaaccagatggagttaatcatcctttgctactagctagacgagctgaaccacagcgtcacaacattgtgtgtgtgtgttgtaagtgtaataatcaactt

SEQ ID NO: 100 - DNA, 120 bp, human papillomavirus
  133448_5511706_gi|9628574.E7|lcl|HPV61_E7.1|Humanpapillomavirus_1_ 2
ttagacagctcagagtcagaggaggaggatagtgtgcgtgagcaacttgttgaacaagcacagcaggcctacagggtggttactacctgtggcatttgtaagtgtccagttaggctggtg

SEQ ID NO: 101 - DNA, 120 bp, human papillomavirus
  133448_5511707_gi|39932599.E7|lcl|HPV62_E7.1|Humanpapillomaviru_1_ 2
agacagctcagaggaagaggtggatgatagggacagtgctgagcaacctgtagaacgggcacagcaggcccatagggtggttactacgtgtggtatttgcaactgtttagttaggttggt

SEQ ID NO: 102 - DNA, 120 bp, human papillomavirus
  133448_5511708_gi|1020290.E7|lcl|HPV66_E7.1|Humanpapillomavirus_1_ 2
gatgaggatgaggatgaaatagaccatttgctggagcggccacagcaagctagacaagctgaacaacataagtgttacctaattcacgtaccttgttgtaagtgtgagttggtggtgcag

SEQ ID NO: 103 - DNA, 120 bp, human papillomavirus
  133448_5511709_gi|3228267.E7|lcl|HPV67_E7.1|Humanpapillomavirus_1_ 2
agctcagatgaggaggatgaagtaggcatggaccggccagatggacaagcaaaaccagatacaaccaattaccatattgttactgtgtgtaacatctgtgagtgcactttgcgtttgtgc

SEQ ID NO: 104 - DNA, 120 bp, human papillomavirus
  133448_5511710_gi|71726685.E7|lcl|HPV68_E7.1|Humanpapillomaviru_1_ 2
acgatgaaatagatgaacccgaccatgcagttaatcaccaccaacatctactactagccagacgggacgaacaacagcgtcacagaattcagtgtctgtgttgtaagtgtaacaaggcac

SEQ ID NO: 105 - DNA, 120 bp, human papillomavirus
  133448_5511711_gi|6970418.E7|lcl|HPV69_E7.1|Humanpapillomavirus_1_ 2
atttgacagctcagaggaggatgaaacagataatgtgcgtaaccagcaagccagacaagctgaacaagaagcgtgttatagaatagaagcggaatgttgtgtatgtaatagtatagtgca

SEQ ID NO: 106 - DNA, 120 bp, human papillomavirus
  133448_5511712_gi|1173493.E7|lcl|HPV70_E7.1|Humanpapillomavirus_1_ 2
gacaatgaaacagatgaacccgaccatgtagttaatcaccaacaacaactactagccagacgggaagaaccacagcgtcacaaaatacagtgtatgtgttgtaagtgtaatactacactg

SEQ ID NO: 107 - DNA, 120 bp, human papillomavirus
  133448_5511713_gi|12084981.E7|lcl|HPV71_E7.1|Humanpapillomaviru_1_ 2
ttgccagctcagacgaggaagataatagggtggacggtgagcaacccacagaaccagcacagcaggcatatagggtggtttcatactgtggtaggtgctgtcgtgcagttaggcttgtgg

SEQ ID NO: 108 - DNA, 120 bp, human papillomavirus
  133448_5511714_gi|1491683.E7|lcl|HPV72_E7.1|Humanpapillomavirus_1_ 2
ctcagagtcagagtcagaggatgagagggacggtgttggtgtgcaggagcaacttgtagaacaagcacagcaggcctacggggtggttactacctgtggcaggtgctaccgtccagttag

SEQ ID NO: 109 - DNA, 120 bp, human papillomavirus
  133448_5511715_gi|1491692.E7|lcl|HPV73_E7.1|Humanpapillomavirus_1_ 2
gacaactcagaggatgaggatgaaacagacagccatctagacagacaagctgaacgagagtgttacagaatagttactgactgcacgaagtgtcagtgcacagtatgccttgccattgaa

SEQ ID NO: 110 - DNA, 120 bp, human papillomavirus
  133448_5511716_gi|40804509.E7|lcl|HPV81_E7.1|Humanpapillomaviru_1_ 2
acagctcagaggaagaggtggatgtggatgatagggacagtggtgagcaactagtagaacaagcacagcaggcctatagggtggttactacgtgtggtatttgtaggtgtttagttaggc

SEQ ID NO: 111 - DNA, 120 bp, human papillomavirus
  133448_5511717_gi|6970427.E7|lcl|HPV82_E7.1|Humanpapillomavirus_1_ 2
ctcagacgaggaggatgaagtagataatatgcgtgaccagccagccagacaagctggacaggatacgtgttacagaattaaagtgcactgttgcaggtgttcgagtgttgtacagctcgc

SEQ ID NO: 112 - DNA, 120 bp, human papillomavirus
  133448_5511718_gi|5059324.E7|lcl|HPV83_E7.1|Humanpapillomavirus_1_ 2
gacagctcagaggaggaggatgatagggactgtggcgtgcgtgaccaactggcagaacaagcaaagcaggcctatagggtgttaacagtgtgtggtatgtgtggccaggcacttagacta

SEQ ID NO: 113 - DNA, 120 bp, human papillomavirus
  133448_5511719_gi|12958167.E7|lcl|HPV84_E7.1|Humanpapillomaviru_1_ 2
agacagctcagaggaggaggataatggggattgcttgcgtgaccaacctgcaaaaccagcacagctggcctatagggtggtaacagagtgtgggttgtgtagccgtccagttaggctggc

SEQ ID NO: 114 - DNA, 120 bp, human papillomavirus
  133448_5511720_gi|50253426.E7|lcl|HPV96_E7.1|Humanpapillomaviru_1_ 2
acagaagagcagtcagaaaacctgtcagaatctacagtagcagaggtggagcctcagagaactctgtacaagattgtagtaccgtgtggaggctgtgattcccggctgaaaatctacgtt

SEQ ID NO: 115 - DNA, 120 bp, human papillomavirus
  129911_5270177_HPV33REF_1_1
gtaaactataatgccaagttttaaaaaagtagggtgtaaccgaaagcggttcaaccgaaaacggtgcatatataaagcaaacattttgcagtaaggtactgcacgactatgtttcaagac

SEQ ID NO: 116 - DNA, 120 bp, human papillomavirus
  129911_5270177_HPV33REF_1_2
acactgaggaaaaaccacgaacattgcatgatttgtgccaagcattggagacaactatacacaacattgaactacagtgcgtggaatgcaaaaaacctttgcaacgatctgaggtatatg

SEQ ID NO: 117 - DNA, 120 bp, human papillomavirus
  129911_5270177_HPV33REF_1_3
tgattttgcatttgcagatttaacagttgtatatagagagggaaatccatttggaatatgtaaactgtgtttgcggttcttatctaaaattagtgaatatagacattataattattctgt

SEQ ID NO: 118 - DNA, 120 bp, human papillomavirus
  129911_5270177_HPV33REF_1_4
gtatatggaaatacattagaacaaacagttaaaaaacctttaaatgaaatattaattaggtgtattatatgtcaaagacctttgtgtcctcaagaaaaaaaacgacatgtggatttaaac

SEQ ID NO: 119 - DNA, 120 bp, human papillomavirus
  129911_5270177_HPV33REF_1_5
acaaacgatttcataatatttcgggtcgttgggcagggcgctgtgcggcgtgttggaggtcccgacgtagagaaactgcactgtgacgtgtaaaaacgccatgagaggacacaagccaac

SEQ ID NO: 120 - DNA, 120 bp, human papillomavirus
  129911_5270177_HPV33REF_1_6
acgttaaaggaatatgttttagatttatatcctgaaccaactgacctatactgctatgagcaattaagtgacagctcagatgaggatgaaggcttggaccggccagatggacaagcacaa

SEQ ID NO: 121 - DNA, 120 bp, human papillomavirus
  129911_5270177_HPV33REF_1_7
aaccagccacagctgattactacattgtaacctgttgtcacacttgtaacaccacagttcgtttatgtgtcaacagtacagcaagtgacctacgaaccatacagcaactacttatgggca

SEQ ID NO: 122 - DNA, 120 bp, human papillomavirus
  129911_5270177_HPV33REF_1_8
cacagtgaatattgtgtgccctacctgtgcacaacaataaacatcatctacaatggccgatcctgaaggtacaaatggggctgggatggggtgtactggttggtttgaggtagaagcagt

SEQ ID NO: 123 - DNA, 120 bp, human papillomavirus
  129911_5270177_HPV33REF_1_9
gtcatagagagaagaacaggagataatatttcagaagatgaggatgaaacagcagatgacagtggcacggatttactagagtttatagatgattctatggaaaatagtatacaggcagac

SEQ ID NO: 124 - DNA, 120 bp, human papillomavirus
  129911_5270177_HPV33REF_1_10
acacagaggcagcccgggcattgtttaatatacaggaaggggaggatgatttaaatgctgtgtgtgcactaaaacgaaagtttgccgcatgttcacaaagtgctgcggaggacgttgttg

SEQ ID NO: 125 - DNA, 120 bp, human papillomavirus
  129911_5270177_HPV33REF_1_11
tgatcgtgctgcaaacccgtgtagaacgtctattaataaaaataaagaatgcacatacagaaaacgaaaaatagatgagctagaagacagcggatatggcaatactgaagtggaaactca

SEQ ID NO: 126 - DNA, 120 bp, human papillomavirus
  129911_5270177_HPV33REF_1_12
cagcagatggtacaacaggtagaaagtcaaaatggcgacacaaacttaaatgacttagaatctagtggggtgggggatgattcagaagtaagctgtgagacaaatgtagatagctgtgaa

SEQ ID NO: 127 - DNA, 120 bp, human papillomavirus
  129911_5270177_HPV33REF_1_13
aaaatgttacgttgcaggaaattagtaatgttctacatagtagtaatacaaaagcaaatatattatataaatttaaagaggcctatggaataagttttatggaattagtaagaccattta

SEQ ID NO: 128 - DNA, 120 bp, human papillomavirus
  129911_5270177_HPV33REF_1_14
taaaagtgataaaacaagctgtacagattggtgtataacaggatatggaattagtccatcagtagcagaaagtttaaaagtattaattaaacagcatagtttgtatactcatttacaatg

SEQ ID NO: 129 - DNA, 120 bp, human papillomavirus
  129911_5270177_HPV33REF_1_15
tgtttaacttgcgatagaggaataataatattattgttaattagatttaggtgtagcaaaaacaggttaacagtagcaaaactaatgagtaatttattatcaatacctgaaacatgtatg

SEQ ID NO: 130 - DNA, 120 bp, human papillomavirus
  129911_5270177_HPV33REF_1_16
tggttatagagccaccaaaattacggagccaaacatgtgcattgtattggtttagaacagcaatgtcaaacattagtgatgtacaaggtacaacacctgaatggatagatagactaactg

SEQ ID NO: 131 - DNA, 120 bp, human papillomavirus
  129911_5270177_HPV33REF_1_17
tgttttacaacatagctttaatgataatatatttgatttaagtgaaatggtacagtgggcatatgataacgagttaacggacgatagtgacattgcatattattatgcacaacttgcaga

SEQ ID NO: 132 - DNA, 120 bp, human papillomavirus
  129911_5270177_HPV33REF_1_18
gattcaaatagtaatgctgctgcatttttaaaaagtaactcacaagcaaaaatagtaaaggactgtggaataatgtgtagacattataaaaaagcagaaaaacgtaaaatgtcaatagga

SEQ ID NO: 133 - DNA, 120 bp, human papillomavirus
  129911_5270177_HPV33REF_1_19
gacaatggatacaaagtagatgtgaaaaaacaaatgatggaggaaattggagaccaatagtacagttgttaagatatcaaaacattgaatttacagcatttttaggtgcatttaaaaagt

SEQ ID NO: 134 - DNA, 120 bp, human papillomavirus
  129911_5270177_HPV33REF_1_20
gtttttaaaaggtataccaaaaaaaagctgtatgctaatttgtggaccagcaaatacaggaaagtcatattttggaatgagtttaatacagtttttaaaagggtgtgttatatcatgtgt

SEQ ID NO: 135 - DNA, 120 bp, human papillomavirus
  129911_5270177_HPV33REF_1_21
gtaaattctaaaagtcacttttggttgcagccattatcagatgcaaaaataggaatgatagatgatgtaacgccaataagttggacatatatagatgattacatgagaaatgcgttagat

SEQ ID NO: 136 - DNA, 120 bp, human papillomavirus
  129911_5270177_HPV33REF_1_22
atggaaatgaaatttcaatagatgtgaaacatagggcattagtgcaattaaaatgtccaccactgcttcttacctcaaatacaaatgcaggcacagactctagatggccatatttacata

SEQ ID NO: 137 - DNA, 120 bp, human papillomavirus
  129911_5270177_HPV33REF_1_23
tagtagattaacagtatttgaatttaaaaatccattcccatttgatgaaaatggtaacccagtgtatgcaataaatgatgaaaattggaaatcctttttctcaaggacgtggtgcaaatt

SEQ ID NO: 138 - DNA, 120 bp, human papillomavirus
  129911_5270177_HPV33REF_1_24
ttagatttaatagaggaagaggacaaggaaaaccatggaggaaatatcagcacgtttaaatgcagtgcaggagaaaatactagatctttacgaagctgataaaactgatttaccatcaca

SEQ ID NO: 139 - DNA, 120 bp, human papillomavirus
  129911_5270177_HPV33REF_1_25
caaattgaacattggaaactgatacgcatggagtgtgctttattgtatacagccaaacaaatgggattttcacatttatgccaccaggtggtgccttctttgttagcatcaaagaccaaa

SEQ ID NO: 140 - DNA, 120 bp, human papillomavirus
  129911_5270177_HPV33REF_1_26
aagcatttcaagtaattgaactacaaatggcattagagacattaagtaaatcacagtatagtacaagccaatggacattgcaacaaacaagcttagaggtgtggctttgtgaaccaccaa

SEQ ID NO: 141 - DNA, 120 bp, human papillomavirus
  129911_5270177_HPV33REF_1_27
aaaatgttttaaaaaacaaggagaaacagtaactgtgcaatatgacaatgacaaaaaaaatacaatggattatacaaactggggtgaaatatatattatagaggaagatacatgtactat

SEQ ID NO: 142 - DNA, 120 bp, human papillomavirus
  129911_5270177_HPV33REF_1_28
atggttacagggaaagtagattatataggtatgtattatatacataactgtgaaaaggtatattttaaatattttaaagaggatgctgcaaagtattctaaaacacaaatgtgggaagta

SEQ ID NO: 143 - DNA, 120 bp, human papillomavirus
  129911_5270177_HPV33REF_1_29
tacatgtgggtggtcaggtaattgtttgtcctacgtctatatctagcaaccaaatatccactactgaaactgctgacatacagacagacaacgataaccgaccaccacaagcagcggcca

SEQ ID NO: 144 - DNA, 120 bp, human papillomavirus
  129911_5270177_HPV33REF_1_30
caaacgacgacgacctgcagacaccacagacaccgcccagccccttacaaagctgttctgtgcagaccccgccttggacaatagaacagcacgtactgcaactaactgcacaaacaagca

SEQ ID NO: 145 - DNA, 120 bp, human papillomavirus
  129911_5270177_HPV33REF_1_31
cagcggactgtgtgtagttctaacgttgcacctatagtgcatttaaaaggtgaatcaaatagtttaaaatgtttaagatacagattaaaaccttataaagagttgtatagttctatgtca

SEQ ID NO: 146 - DNA, 120 bp, human papillomavirus
  129911_5270177_HPV33REF_1_32
catccacctggcattggaccagtgacaacaaaaatagtaaaaatggaattgtaactgtaacatttgtaactgaacagcaacaacaaatgtttttaggtaccgtaaaaataccacctactg

SEQ ID NO: 147 - DNA, 113 bp, human papillomavirus
  129911_5270177_HPV33REF_1_33
tgtgcaaataagtactggatttatgacattataagtgtacatcacaagccaatatgtgctgctaattgtatataaccatgatatttgtttttgtattatgttttatattgttt

SEQ ID NO: 148 - DNA, 120 bp, human papillomavirus
  129911_5270178_HPV33REF_2_1
tatgcttatccttattattacgtcctttaatactttccatttctacctatgcttggttgctggtgttggtattgctgctttgggtgtttgtgggatctcctttaaaaatttttttttgct

SEQ ID NO: 149 - DNA, 120 bp, human papillomavirus
  129911_5270178_HPV33REF_2_2
ctatttgttgtttttatatttaccaatgatgtgtattaattttcatgcacagcatatgacacaacaagagtaatgtatatacatgtatatattgtttgtatatatgtgcacatggtggtg

SEQ ID NO: 150 - DNA, 120 bp, human papillomavirus
  129911_5270178_HPV33REF_2_3
tgttttaacattgttgttgttattttagtttttttttttttgtattactaataaatacctttatattttagcagtgtattattatgagacacaaacgatctacaaggcgcaagcgtgcat

SEQ ID NO: 151 - DNA, 120 bp, human papillomavirus
  129911_5270178_HPV33REF_2_4
atctgcaacacaactataccaaacatgcaaggccacaggcacctgcccacccgatgttattcctaaagtggaaggaagtaccatagcagatcaaattcttaaatatggcagtttaggggt

SEQ ID NO: 152 - DNA, 120 bp, human papillomavirus
  129911_5270178_HPV33REF_2_5
gttttttttggtggtttaggtattggcacaggctctggttcaggtggaaggactggctatgtacctattggtactgacccacctacagctgcaatccccttgcagcctatacgtcctccg

SEQ ID NO: 153 - DNA, 120 bp, human papillomavirus
  129911_5270178_HPV33REF_2_6
cggttactgtagacactgttggacctttagactcgtctatagtgtcattaatagaagaaacaagttttatagaggcaggtgcaccagccccatctattcctacaccatcaggttttgatg

SEQ ID NO: 154 - DNA, 120 bp, human papillomavirus
  129911_5270178_HPV33REF_2_7
tgttactacatctgcagatactacacctgcaattattaatgtttcatctgttggggagtcatctattcaaactatttctacacatttaaatcccacatttactgaaccatctgtactaca

SEQ ID NO: 155 - DNA, 120 bp, human papillomavirus
  129911_5270178_HPV33REF_2_8
caccctccagcgcctgcagaagcctctggacattttatattttcttcccctactgttagcacacaaagttatgaaaacataccaatggatacctttgttgtttccacagacagtagtaat

SEQ ID NO: 156 - DNA, 120 bp, human papillomavirus
  129911_5270178_HPV33REF_2_9
atgtaacatcaagcacgcccattccagggtctcgccctgtggcacgccttggtttatatagtcgcaatacccaacaggttaaggttgttgaccctgcttttttaacatcgcctcataaac

SEQ ID NO: 157 - DNA, 120 bp, human papillomavirus
  129911_5270178_HPV33REF_2_10
acttataacatatgataatcctgcatttgaaagctttgaccctgaagacacattacaatttcaacatagtgatatatcacctgctcctgatcctgactttctagatattattgcattaca

SEQ ID NO: 158 - DNA, 120 bp, human papillomavirus
  129911_5270178_HPV33REF_2_11
cataggcctgctattacatctcgtagacatactgtgcgttttagtagagtaggtcaaaaagccacacttaaaactcgcagtggtaaacaaattggagctagaatacattattatcaggat

SEQ ID NO: 159 - DNA, 120 bp, human papillomavirus
  129911_5270178_HPV33REF_2_12
atttaagtcctattgtgcctttagaccacaccgtgccaaatgaacaatatgaattacagcctttacatgatacttctacatcgtcttatagtattaatgatggtttgtatgatgtttatg

SEQ ID NO: 160 - DNA, 120 bp, human papillomavirus
  129911_5270178_HPV33REF_2_13
tgctgacgatgtggataatgtacacaccccaatgcaacactcatacagtacgtttgcaacaacacgtaccagcaatgtgtctatacctttaaatacaggatttgatactcctgttatgtc

SEQ ID NO: 161 - DNA, 120 bp, human papillomavirus
  129911_5270178_HPV33REF_2_14
tctggccctgatataccttcccctttatttcccacatctagcccatttgttcctatttcgcctttttttccttttgacaccattgttgtagacggtgctgactttgttttacatcctagt

SEQ ID NO: 162 - DNA, 120 bp, human papillomavirus
  129911_5270178_HPV33REF_2_15
gttattttattttacgtcgcaggcgtaaacgttttccatatttttttacagatgtccgtgtggcggcctagtgaggccacagtgtacctgcctcctgtacctgtatctaaagttgtcagc

SEQ ID NO: 163 - DNA, 120 bp, human papillomavirus
  129911_5270178_HPV33REF_2_16
gcactgatgaatatgtgtctcgcacaagcatttattattatgctggtagttccagacttcttgctgttggccatccatatttttctattaaaaatcctactaacgctaaaaaattattgg

SEQ ID NO: 164 - DNA, 120 bp, human papillomavirus
  129911_5270178_HPV33REF_2_17
ggtacccaaagtatcaggcttgcaatatagggtttttagggtccgtttaccagatcctaataaatttggatttcctgacacctccttttataaccctgatacacaacgattagtatgggc

SEQ ID NO: 165 - DNA, 120 bp, human papillomavirus
  129911_5270178_HPV33REF_2_18
gcatgtgtaggccttgaaataggtagagggcagccattaggcgttggcataagtggtcatcctttattaaacaaatttgatgacactgaaaccggtaacaagtatcctggacaaccgggt

SEQ ID NO: 166 - DNA, 120 bp, human papillomavirus
  129911_5270178_HPV33REF_2_19
gtgctgataatagggaatgtttatccatggattataaacaaacacagttatgtttacttggatgtaagcctccaacaggggaacattggggtaaaggtgttgcttgtactaatgcagcac

SEQ ID NO: 167 - DNA, 120 bp, human papillomavirus
  129911_5270178_HPV33REF_2_20
acctgccaatgattgtccacctttagaacttataaatactattattgaggatggtgatatggtggacacaggatttggttgcatggattttaaaacattgcaggctaataaaagtgatgt

SEQ ID NO: 168 - DNA, 120 bp, human papillomavirus
  129911_5270178_HPV33REF_2_21
gttcctattgatatttgtggcagtacatgcaaatatccagattatttaaaaatgactagtgagccttatggtgatagtttatttttctttcttcgacgtgaacaaatgtttgtaagacac

SEQ ID NO: 169 - DNA, 120 bp, human papillomavirus
  129911_5270178_HPV33REF_2_22
acttttttaatagggctggtacattaggagaggctgttcccgatgacctgtacattaaaggttcaggaactactgcctctattcaaagcagtgctttttttcccactcctagtggatcaa

SEQ ID NO: 170 - DNA, 120 bp, human papillomavirus
  129911_5270178_HPV33REF_2_23
aatggttacttccgaatctcagttatttaataagccatattggctacaacgtgcacaaggtcataataatggtatttgttggggcaatcaggtatttgttactgtggtagataccactcg

SEQ ID NO: 171 - DNA, 120 bp, human papillomavirus
  129911_5270178_HPV33REF_2_24
cgcagtactaatatgactttatgcacacaagtaactagtgacagtacatataaaaatgaaaattttaaagaatatataagacatgttgaagaatatgatctacagtttgtttttcaacta

SEQ ID NO: 172 - DNA, 120 bp, human papillomavirus
  129911_5270178_HPV33REF_2_25
tatgcaaagttaccttaactgcagaagttatgacatatattcatgctatgaatccagatattttagaagattggcaatttggtttaacacctcctccatctgctagtttacaggatacct

SEQ ID NO: 173 - DNA, 120 bp, human papillomavirus
  129911_5270178_HPV33REF_2_26
ctataggtttgttacctctcaggctattacgtgtcaaaaaacagtacctccaaaggaaaaggaagaccccttaggtaaatatacattttgggaagtggatttaaaggaaaaattttcagc

SEQ ID NO: 174 - DNA, 120 bp, human papillomavirus
  129911_5270178_HPV33REF_2_27
gcagatttagatcagtttcctttgggacgcaagtttttattacaggcaggtcttaaagcaaaacctaaacttaaacgtgcagcccccacatccacccgcacatcgtctgcaaaacgcaaa

SEQ ID NO: 175 - DNA, 120 bp, human papillomavirus
  129911_5270178_HPV33REF_2_28
aaaaggttaaaaaataacactttgtgtaattgtgttatgttgttgttttgttctgtctatgtactttgtgttgttgtgttgtgttgttgtttgttttttgtgtatgtgttacaatgtatg

SEQ ID NO: 176 - DNA, 120 bp, human papillomavirus
  129911_5270178_HPV33REF_2_29
tgttatgttgtatgttactgtgtttgttttatgtgtacttgtttgtgtgcatgttctatgtacttgtcagtttcctgtttgtgtatatgttaataaaacattgtgtgtatttgttaaact

SEQ ID NO: 177 - DNA, 120 bp, human papillomavirus
  129911_5270178_HPV33REF_2_30
ctatttgtatgtatgttatgtatatgggtgtacctatatgagtaaggagttgtattgcttgccctaccctgcattgcaatgtacctacctttatttccctatatttgtagtacctacatg

SEQ ID NO: 178 - DNA, 120 bp, human papillomavirus
  129911_5270178_HPV33REF_2_31
tgtttagtattgctttaccttttgacatactagtgtccatattgtacaatttcctccattttgtatgcctaaccgttttcggttacttggcatacataccctatgacattggcagaacag

SEQ ID NO: 179 - DNA, 120 bp, human papillomavirus
  129911_5270178_HPV33REF_2_32
agttaatccttttctttcctgcactgtgtttgtctgtacttgctgcattggcatacataccctatgacattggcagaacagttaatccttttctttcctgcactgtgtttgtctgtactt

SEQ ID NO: 180 - DNA, 120 bp, human papillomavirus
  129911_5270178_HPV33REF_2_33
ttgctgcattgactcatatatacatgcagtgcaattgcaaaatacttaattgtactaatagtttacacatgcttttaggcacatatttttactttactttcaaaccttaagtgcagtttt

SEQ ID NO: 181 - DNA, 120 bp, human papillomavirus
  129911_5270178_HPV33REF_2_34
ttggcttacacaattgctttgtatgccaaactatgccttgtaaaagtgagtcactacctgtttattaccaggtgtggactaaccgttttaggtcatattggtcatttataatcttttata

SEQ ID NO: 182 - DNA, 120 bp, human papillomavirus
  129911_5270179_HPV45REF_1_1
aatacttttaacaattatactacataaaaaagggtgtaaccgaaaacggttgcaaccaaaaacggtgcatataaaagctttgtggaaaagtgcattacaggatggcgcgctttgacgatc

SEQ ID NO: 183 - DNA, 120 bp, human papillomavirus
  129911_5270179_HPV45REF_1_2
caaagcaacgaccctacaagctaccagatttgtgcacagaattgaatacatcactacaagacgtatctattgcctgtgtatattgcaaagcaacattggaacgcacagaggtatatcaat

SEQ ID NO: 184 - DNA, 120 bp, human papillomavirus
  129911_5270179_HPV45REF_1_3
ttgcttttaaagatttatgtatagtgtatagagactgtatagcatatgctgcatgccataaatgtatagacttttattccagaattagagaattaagatattattcaaactctgtatatg

SEQ ID NO: 185 - DNA, 120 bp, human papillomavirus
  129911_5270179_HPV45REF_1_4
gagagacactggaaaaaataactaatacagagttgtataatttgttaataaggtgcctgcggtgccagaaaccattgaacccagcagaaaaacgtagacaccttaaggacaaacgaagat

SEQ ID NO: 186 - DNA, 120 bp, human papillomavirus
  129911_5270179_HPV45REF_1_5
ttcacagcatagctggacagtaccgagggcagtgtaatacatgttgtgaccaggcacggcaagaaagacttcgcagacgtagggaaacacaagtatagcaataagtatgcatggaccccg

SEQ ID NO: 187 - DNA, 120 bp, human papillomavirus
  129911_5270179_HPV45REF_1_6
ggaaacactgcaagaaattgtattgcatttggaacctcagaatgaattagatcctgttgacctgttgtgttacgagcaattaagcgagtcagaggaggaaaacgatgaagcagatggagt

SEQ ID NO: 188 - DNA, 120 bp, human papillomavirus
  129911_5270179_HPV45REF_1_7
tagtcatgcacaactaccagcccgacgagccgaaccacagcgtcacaaaattttgtgtgtatgttgtaagtgtgacggcagaattgagcttacagtagagagctcggcagaggaccttag

SEQ ID NO: 189 - DNA, 120 bp, human papillomavirus
  129911_5270179_HPV45REF_1_8
aacactacagcagctgtttttgagcaccttgtcctttgtgtgtccgtggtgtgcaactaaccaataatctacaatggcggatccagaaggtaccgacggggagggaacggggtgtaatgg

SEQ ID NO: 190 - DNA, 120 bp, human papillomavirus
  129911_5270179_HPV45REF_1_9
ctggttctttgtagaaacaattgtagagaaaaaaacaggggatgtaatatcagatgatgaggatgaaactgcaacagatacagggtcggatatggtagattttattgacacacaattatc

SEQ ID NO: 191 - DNA, 120 bp, human papillomavirus
  129911_5270179_HPV45REF_1_10
catttgtgaacaggcagagcaagagacagcacaggcattgttccatgcgcaggaagttcagaatgatgcacaggtgttgcatcttttaaaacgaaagtttgcaggaggcagcaaggaaaa

SEQ ID NO: 192 - DNA, 120 bp, human papillomavirus
  129911_5270179_HPV45REF_1_11
cagtccattaggggagcagctaagtgtggatacggatctaagtccacggttacaagaaatttcattaaatagtgggcacaaaaaagcaaaacgacggttgtttacaatatcagatagtgg

SEQ ID NO: 193 - DNA, 120 bp, human papillomavirus
  129911_5270179_HPV45REF_1_12
ctatggctgttctgaagtggaagctgcagagactcaggtaactgtaaacactaatgcggaaaatggcggcagtgtacatagtacacaaagtagtggtggggatagtagtgacaatgcaga

SEQ ID NO: 194 - DNA, 120 bp, human papillomavirus
  129911_5270179_HPV45REF_1_13
aaatgtagatccgcattgcagtattacagaactaaaggagctattacaagcaagtaacaaaaaggctgcaatgctggcagtatttaaagacatatatgggctgtcatttacggatttggt

SEQ ID NO: 195 - DNA, 120 bp, human papillomavirus
  129911_5270179_HPV45REF_1_14
tagaaattttaaaagtgataaaacaacatgtacagattgggtaatggctatatttggagttaatccaacggtagcagaaggctttaaaacattaattaaaccagcaacgttatacgccca

SEQ ID NO: 196 - DNA, 120 bp, human papillomavirus
  129911_5270179_HPV45REF_1_15
tatccaatgtttagattgtaaatggggagtattaatattagctttattaagatataaatgtggcaaaaatagactaactgttgcaaaaggcttaagcacattgttgcacgtacctgaaac

SEQ ID NO: 197 - DNA, 120 bp, human papillomavirus
  129911_5270179_HPV45REF_1_16
atgtatgttaattgaaccaccaaaattgcgaagtagtgttgcagcattatactggtatagaacaggtatatccaatattagtgaagtaagtggagacacacctgagtggatacaaagact

SEQ ID NO: 198 - DNA, 120 bp, human papillomavirus
  129911_5270179_HPV45REF_1_17
gacaattattcaacatggtattgacgatagtaattttgatttgtcagacatggtgcaatgggcatttgataatgaccttacagatgaaagtgatatggcatttcaatatgcccaattagc

SEQ ID NO: 199 - DNA, 120 bp, human papillomavirus
  129911_5270179_HPV45REF_1_18
agactgcaacagtaatgcagctgcatttttaaaaagtaactgccaagccaaatatttaaaagattgtgctgtaatgtgtagacattataaaagagcacaaaaacgccaaatgaatatgtc

SEQ ID NO: 200 - DNA, 120 bp, human papillomavirus
  129911_5270179_HPV45REF_1_19
tcaatggattaaatatagatgttccaaaatagatgaaggtggggattggagacccatagtacaattcctaagatatcagggagtagaatttattagctttttaagggcactaaaggaatt

SEQ ID NO: 201 - DNA, 120 bp, human papillomavirus
  129911_5270179_HPV45REF_1_20
tcttaaaggaacaccaaaaaaaaattgtatactgttatatggacctgcaaatacaggaaaatcgtattttggaatgagttttatacatttcctacaaggtgcaataatatcatttgtaaa

SEQ ID NO: 202 - DNA, 120 bp, human papillomavirus
  129911_5270179_HPV45REF_1_21
ttcaaacagccatttttggttagaaccgttagcagatactaaggtagccatgttggatgatgccacacacacgtgttggacatattttgataattatatgagaaatgcattagatggtaa

SEQ ID NO: 203 - DNA, 120 bp, human papillomavirus
  129911_5270179_HPV45REF_1_22
tcctataagtatagacagaaagcataaaccattattacagctaaaatgtcctccaatcctattaacatccaatattgatccagcaaaagataataaatggccatatttagaaagtagggt

SEQ ID NO: 204 - DNA, 120 bp, human papillomavirus
  129911_5270179_HPV45REF_1_23
gacggtatttacatttccacatgcatttccatttgataaaaatggtaatccagtatatgaaataaatgataaaaattggaaatgtttttttgaaaggacatggtccagattagatttgca

SEQ ID NO: 205 - DNA, 120 bp, human papillomavirus
  129911_5270179_HPV45REF_1_24
cgaggacgatgaagatgcagacaccgaaggaatccctttcggaacgtttaagtgcgttacaggacaaaatactagaccactatgaaaatgacagtaaagacataaacagccaaataagtt

SEQ ID NO: 206 - DNA, 120 bp, human papillomavirus
  129911_5270179_HPV45REF_1_25
attggcaacttatacgtttggaaaatgcaatactatttacagcaagggaacatggtattaccaaactaaaccaccaggtggtgcctcctattaacatttcaaaaagcaaagcacataaag

SEQ ID NO: 207 - DNA, 120 bp, human papillomavirus
  129911_5270179_HPV45REF_1_26
ctattgaactgcaaatggccttaaagggccttgcacaaagcaagtataacaatgaggaatggacactgcaagatacatgcgaggaactatggaatacagaaccgtcgcagtgttttaaaa

SEQ ID NO: 208 - DNA, 120 bp, human papillomavirus
  129911_5270179_HPV45REF_1_27
aaggcggtaaaaccgtgcacgtatactttgatggcaacaaggacaactgtatgaactatgtagtatgggacagtatatattatataactgagacagggatatgggacaaaacagcagcat

SEQ ID NO: 209 - DNA, 120 bp, human papillomavirus
  129911_5270179_HPV45REF_1_28
gtgttagctattggggtgtatattatataaaagatggagataccacatattatgtacaatttaaaagcgaatgtgagaaatatggaaatagtaatacgtgggaagtacaatatgggggca

SEQ ID NO: 210 - DNA, 120 bp, human papillomavirus
  129911_5270179_HPV45REF_1_29
atgtaattgattgtaatgactctatgtgcagtaccagtgacgacacggtatccgctactcagattgttagacagctacaacacgcctccacgtcgacccccaaaaccgcatccgtgggca

SEQ ID NO: 211 - DNA, 120 bp, human papillomavirus
  129911_5270179_HPV45REF_1_30
ccccaaaaccccacatccagacgccggctactaagcgacctagacagtgtggactcacagagcagcaccacggacgtgtcaacacccacgtgcacaacccgctcctgtgttcaagtacaa

SEQ ID NO: 212 - DNA, 120 bp, human papillomavirus
  129911_5270179_HPV45REF_1_31
gtaacaacaaaagaaggaaagtgtgtagtggtaacactacgcctataatacacttaaaaggtgacaaaaacagtttgaaatgtttaagatataggctacgcaaatatgcagaccattact

SEQ ID NO: 213 - DNA, 120 bp, human papillomavirus
  129911_5270179_HPV45REF_1_32
cagaaatatcctccacctggcattggacaggttgtaataaaaacactggtatattaactgtaacatataatagtgaggtacaaagaaatacctttttggatgtagttactattcctaaca

SEQ ID NO: 214 - DNA, 120 bp, human papillomavirus
  129911_5270179_HPV45REF_1_33
gtgtacaaatctcggtgggatacatgactatatgaatctgtatattgtatacagtatgtaacattactatgctatctttagtgtttttattgtgcttttctgtgtgcctttatgtgtgct

SEQ ID NO: 215 - DNA, 120 bp, human papillomavirus
  129911_5270179_HPV45REF_1_34
gcaatgtcccgcttgtgcagtctgtctatgtgtgtgcttttgcttggttgttggtgtttctttttatagttgttattacatccccattaacagcatttgctgtatacatttgttgctatt

SEQ ID NO: 216 - DNA, 118 bp, human papillomavirus
  129911_5270179_HPV45REF_1_35
tactacctatgtttgtattacatatgcatgctttacacaccatacaataattactataatgtacagtacagtgtaacatacctgtgatgtgcatgttgttgtatttttgtatttttgt

SEQ ID NO: 217 - DNA, 120 bp, human papillomavirus
  129911_5270180_HPV45REF_2_1
tttttgtatttttgtattttatatgtttaataaaccatggtatcccaccgtgcagcacgtcgcaagcgggcctctgcaactgacttatatagaacatgtaagcaatccggtacgtgcccc

SEQ ID NO: 218 - DNA, 120 bp, human papillomavirus
  129911_5270180_HPV45REF_2_2
ccctgatgttattaacaaagtggaaggcacaaccttagctgataaaattttacagtggtctagccttgggatatttttgggtggccttggcattggtaccggcagtggttctggaggccg

SEQ ID NO: 219 - DNA, 120 bp, human papillomavirus
  129911_5270180_HPV45REF_2_3
gtacgggctatgtacccttagggggcaggtctaatactgttgtggatgttggccccactaggccacctgtggttattgaacctgtagggcctactgatccatctattgttacgttggtag

SEQ ID NO: 220 - DNA, 120 bp, human papillomavirus
  129911_5270180_HPV45REF_2_4
gaggattccagtgttgttgcctctggtgctccggttcccacatttaccggaacctctgggtttgaaattacgtcttctggtactaccacaccagctgtgttggacatcacacctaccgtg

SEQ ID NO: 221 - DNA, 120 bp, human papillomavirus
  129911_5270180_HPV45REF_2_5
ggactctgtttctatttcgtcaactagttttacaaatcctgcattttctgatccctctattattgaggtgccccaaacaggggaggtatcaggtaatatatttgttggtacaccaacatc

SEQ ID NO: 222 - DNA, 120 bp, human papillomavirus
  129911_5270180_HPV45REF_2_6
cgggcagccatggatatgaggaaatacctttacaaacatttgcatcttctgggtcaggtacggaacccattagtagtacccccctccctactgtgcggcgggtacggggtccccgcctgt

SEQ ID NO: 223 - DNA, 120 bp, human papillomavirus
  129911_5270180_HPV45REF_2_7
tatagtagggctaatcaacaggtccgtgtgtccacctcacagtttttaacacatccctcatcgttggttacatttgataatccagcttatgagcccctggacaccacactatcctttgag

SEQ ID NO: 224 - DNA, 120 bp, human papillomavirus
  129911_5270180_HPV45REF_2_8
gcctaccagtaatgttcctgattccgattttatggatattattcgtttgcataggccagcattatcctctagacgtggcactgttagatttagtagattgggtcaaagggcaaccatgtt

SEQ ID NO: 225 - DNA, 120 bp, human papillomavirus
  129911_5270180_HPV45REF_2_9
ttacacgtagtggtaaacaaatagggggtagggtacatttttaccatgatataagccccattgctgctacagaggaaattgaattgcagcctttaattagtgctacaaatgatagtgacc

SEQ ID NO: 226 - DNA, 120 bp, human papillomavirus
  129911_5270180_HPV45REF_2_10
ctgtttgatgtatatgcagacttcccacctcctgcgtccactacacctagcactatacacaaatcatttacatatccaaagtattccttgaccatgccttctactgctgcatcctcttac

SEQ ID NO: 227 - DNA, 120 bp, human papillomavirus
  129911_5270180_HPV45REF_2_11
cagtaatgttacagtaccattaacatctgcatgggatgtacctatatatactggcccggacattatattgccatcccatactcctatgtggcctagtacatctcctaccaatgcttccac

SEQ ID NO: 228 - DNA, 120 bp, human papillomavirus
  129911_5270180_HPV45REF_2_12
ccaccacctatataggtattcatggcacacaatattatttatggccatggtattattattttcctaaaaaacgtaaacgtattccctatttttttgcagatggctttgtggcggcctagt

SEQ ID NO: 229 - DNA, 120 bp, human papillomavirus
  129911_5270180_HPV45REF_2_13
tgacagtacggtatatcttccaccaccttctgtggccagagttgtcagcactgatgattatgtgtctcgcacaagcatattttatcatgcaggcagttcccgattattaactgtaggcaa

SEQ ID NO: 230 - DNA, 120 bp, human papillomavirus
  129911_5270180_HPV45REF_2_14
atccatattttagggttgtacctaatggtgcaggtaataaacaggctgttcctaaggtatccgcatatcagtatagggtgtttagagtagctttacccgatcctaataaatttggattac

SEQ ID NO: 231 - DNA, 120 bp, human papillomavirus
  129911_5270180_HPV45REF_2_15
cctgattctactatatataatcctgaaacacaacgtttggtttgggcatgtgtaggtatggaaattggtcgtgggcagcctttaggtattggcctaagtggccatccattttataataaa

SEQ ID NO: 232 - DNA, 120 bp, human papillomavirus
  129911_5270180_HPV45REF_2_16
attggatgatacagaaagtgctcatgcagctacagctgttattacgcaggatgttagggataatgtgtcagttgattataagcaaacacagctgtgtattttaggttgtgtacctgctat

SEQ ID NO: 233 - DNA, 120 bp, human papillomavirus
  129911_5270180_HPV45REF_2_17
ttggtgagcactgggccaagggcacactttgtaaacctgcacaattgcaacctggtgactgtcctcctttggaacttaaaaacaccattattgaggatggtgatatggtggatacaggtt

SEQ ID NO: 234 - DNA, 120 bp, human papillomavirus
  129911_5270180_HPV45REF_2_18
tatggggcaatggattttagtacattgcaggatacaaagtgcgaggttccattagacatttgtcaatccatctgtaaatatccagattatttgcaaatgtctgctgatccctatggggat

SEQ ID NO: 235 - DNA, 120 bp, human papillomavirus
  129911_5270180_HPV45REF_2_19
ttctatgtttttttgcctacgccgtgaacaactgtttgcaagacatttttggaatagggcaggtgttatgggtgacacagtacctacggacctatatattaaaggcactagcgctaatat

SEQ ID NO: 236 - DNA, 120 bp, human papillomavirus
  129911_5270180_HPV45REF_2_20
tgcgtgaaacccctggcagttgtgtgtattccccttctcccagtggctctattattacttctgattctcaattatttaataagccatattggttacataaggcccagggccataacaatg

SEQ ID NO: 237 - DNA, 120 bp, human papillomavirus
  129911_5270180_HPV45REF_2_21
ggtatttgttggcataatcagttgtttgttactgtagtggacactacccgcagtactaatttaacattatgtgcctctacacaaaatcctgtgccaagtacatatgaccctactaagttt

SEQ ID NO: 238 - DNA, 120 bp, human papillomavirus
  129911_5270180_HPV45REF_2_22
taagcagtatagtagacatgtggaggaatatgatttacagtttatttttcagttgtgcactattactttaactgcagaggttatgtcatatatccatagtatgaatagtagtatattaga

SEQ ID NO: 239 - DNA, 120 bp, human papillomavirus
  129911_5270180_HPV45REF_2_23
aaaattggaattttggtgtccctccaccacctactacaagtttggtggatacatatcgttttgtgcaatcagttgctgttacctgtcaaaaggatactacacctccagaaaagcaggatc

SEQ ID NO: 240 - DNA, 120 bp, human papillomavirus
  129911_5270180_HPV45REF_2_24
ccatatgataaattaaagttttggactgttgacctaaaggaaaaattttcctccgatttggatcaatatccccttggtcgaaagtttttagttcaggctgggttacgtcgtaggcctacc

SEQ ID NO: 241 - DNA, 120 bp, human papillomavirus
  129911_5270180_HPV45REF_2_25
cataggacctcgtaagcgtcctgctgcttccacgtctactgcatctactgcatctaggcctgccaaacgtgtacgtatacgtagtaagaaataatatgttagcacatatatgtatgtttg

SEQ ID NO: 242 - DNA, 120 bp, human papillomavirus
  129911_5270181_HPV45REF_3_1
gaatgtgccttgtggcatgtatggtgttactgtacataattgtggtattaaataaagtatgctaatagtgttgtgtagggttgcacccttgtgagtaacaatactatttgtgtgtatgtg

SEQ ID NO: 243 - DNA, 120 bp, human papillomavirus
  129911_5270181_HPV45REF_3_2
tattgctttgtaccctatattctttcctgtatttcaagttataaacttgcatactacacagcatccattttacttataatcctccattttgctgtgcaaccgatttcggttgcctgtggc

SEQ ID NO: 244 - DNA, 120 bp, human papillomavirus
  129911_5270181_HPV45REF_3_3
ttatatgtgaccttttaaacataatacctaaactggcacatttacaacccctacatagtttaacctactggcgcgccttcttggcgtacatgtggcacacctggtattagtcattttcct

SEQ ID NO: 245 - DNA, 120 bp, human papillomavirus
  129911_5270181_HPV45REF_3_4
gtccaggtgtactaaaacaatggcttgcacaactgtatccacaccctatgtaataaaactgcttttaggcacatattttagtctgtttttacctgtgctaattgtataattggcgtgtag

SEQ ID NO: 246 - DNA, 119 bp, human papillomavirus
  129911_5270181_HPV45REF_3_5
aaccactttcttatccaacaatctgtctacttgttacataaactataaactgactcacttatacatacatagtttatgcaaccgaaaaaggttgggccctataacacataccttttctt

SEQ ID NO: 247 - DNA, 120 bp, human papillomavirus
  133445_5511603_HPV52REF.1(HPV52)_1_1
taaattataatcttatactagtaaaaaatagggtgtaaccgaaaacggtcagaccgaaaccggtgtatatatatagaacacagtgtagctaacgcacggccatgtttgaggatccagcaa

SEQ ID NO: 248 - DNA, 120 bp, human papillomavirus
  133445_5511603_HPV52REF.1(HPV52)_1_2
cacgaccccggaccctgcacgaattgtgtgaggtgctggaagaatcggtgcatgaaataaggctgcagtgtgtgcagtgcaaaaaagagctacaacgaagagaggtatacaagtttctat

SEQ ID NO: 249 - DNA, 120 bp, human papillomavirus
  133445_5511603_HPV52REF.1(HPV52)_1_3
ttacagatttacgaatagtatatagagacaataatccatatggcgtgtgtattatgtgcctacgctttttatctaagataagtgaatataggcattatcaatattcactgtatgggaaaa

SEQ ID NO: 250 - DNA, 120 bp, human papillomavirus
  133445_5511603_HPV52REF.1(HPV52)_1_4
cattagaagagagggtaaaaaaaccattaagtgaaataactattagatgtataatttgtcaaacgccattatgtcctgaagaaaaagaaagacatgttaatgcaaacaagcgatttcata

SEQ ID NO: 251 - DNA, 120 bp, human papillomavirus
  133445_5511603_HPV52REF.1(HPV52)_1_5
atattatgggtcgttggacagggcgctgttcagagtgttggagaccccgacctgtgacccaagtgtaacgtcatgcgtggagacaaagcaactataaaagattatatattagatctgcaa

SEQ ID NO: 252 - DNA, 120 bp, human papillomavirus
  133445_5511603_HPV52REF.1(HPV52)_1_6
cctgaaacaactgacctacactgctatgagcaattaggtgacagctcagatgaggaggatacagatggtgtggaccggccagatggacaagcagaacaagccacaagcaattactacatt

SEQ ID NO: 253 - DNA, 120 bp, human papillomavirus
  133445_5511603_HPV52REF.1(HPV52)_1_7
gtgacatattgtcacagttgtgatagcacactacggctatgcattcatagcactgcgacggaccttcgtactctacagcaaatgctgttgggcacattacaagttgtgtgccccggctgt

SEQ ID NO: 254 - DNA, 120 bp, human papillomavirus
  133445_5511603_HPV52REF.1(HPV52)_1_8
gcacggctataaacaaccctgcaatggaggaccctgaaggtacagagggcgaaagggagggatgtacaggctggtttgaagtagaggcaataatagaaaaacaaacaggagataacattt

SEQ ID NO: 255 - DNA, 120 bp, human papillomavirus
  133445_5511603_HPV52REF.1(HPV52)_1_9
cagaggacgaggatgaaaatgcatatgatagtggaacagatctaatagattttatagatgattcaaatataaataatgaacaggcagaacatgaggcagcccgggcattgtttaatgcac

SEQ ID NO: 256 - DNA, 120 bp, human papillomavirus
  133445_5511603_HPV52REF.1(HPV52)_1_10
aggaaggggaggatgatttacatgctgtgtctgcagtaaaacgaaagtttacaagcagtccggaaagtgctgggcaagatggtgtagaaaaacatggtagtccgcgtgcaaaacacattt

SEQ ID NO: 257 - DNA, 120 bp, human papillomavirus
  133445_5511603_HPV52REF.1(HPV52)_1_11
gtgtaaatacagagtgtgttttaccaaaacgcaaaccatgtcacgtagaagacagcggctatggcaatagtgaagtggaagcgcagcagatggcagaccaggtagacgggcaaaatggcg

SEQ ID NO: 258 - DNA, 120 bp, human papillomavirus
  133445_5511603_HPV52REF.1(HPV52)_1_12
actggcaaagtaacagtagtcaatcaagtggggtgggggctagtaattcagatgtaagttgtactagtatagaggacaatgaggaaaatagtaatagaacgctaaaaagcatacaaaata

SEQ ID NO: 259 - DNA, 120 bp, human papillomavirus
  133445_5511603_HPV52REF.1(HPV52)_1_13
ttatgtgcgaaaatagcataaaaacaactgtattatttaaatttaaagaaacatatggtgttagctttatggaattagtaagaccatttaaaagtaatagaagtagttgtacagattggt

SEQ ID NO: 260 - DNA, 120 bp, human papillomavirus
  133445_5511603_HPV52REF.1(HPV52)_1_14
gtattataggaatgggagtaacaccatcagttgcagaaggattaaaagtattaatacagccctatagcatatatgcccatttgcaatgtttaacatgtgacagaggcgtgcttatactgc

SEQ ID NO: 261 - DNA, 120 bp, human papillomavirus
  133445_5511603_HPV52REF.1(HPV52)_1_15
tgctaattaggtttaaatgtggaaaaaacagattaacagtgtccaaactaatgtcacagctgttaaatataccagaaacacatatggtaatagaaccaccaaaattacgaagtgctacct

SEQ ID NO: 262 - DNA, 120 bp, human papillomavirus
  133445_5511603_HPV52REF.1(HPV52)_1_16
gtgcattatattggtatagaacaggtttgtctaatattagtgaggtatatggtaccaccccagaatggatagaacaacaaacagtattacagcatagctttgacaatagcatattcgatt

SEQ ID NO: 263 - DNA, 120 bp, human papillomavirus
  133445_5511603_HPV52REF.1(HPV52)_1_17
ttggagaaatggtgcaatgggcatatgatcatgatataacagatgatagtgacatagcatataaatatgcacagttagcagatgtaaatagcaatgctgcagcattcctaaaaagcaatt

SEQ ID NO: 264 - DNA, 120 bp, human papillomavirus
  133445_5511603_HPV52REF.1(HPV52)_1_18
cgcaagcaaaaatagtaaaggactgtgcaaccatgtgtagacattataaacgggcagaaagaaaacatatgaatattggacaatggatacagtatagatgtgatagaatagatgatggtg

SEQ ID NO: 265 - DNA, 120 bp, human papillomavirus
  133445_5511603_HPV52REF.1(HPV52)_1_19
gagattggaggcctatagtaagatttttaagatatcaagacatagaatttacagcctttttagacgcatttaaaaaatttttaaaaggtatacctaaaaaaaattgtttagtattatatg

SEQ ID NO: 266 - DNA, 120 bp, human papillomavirus
  133445_5511603_HPV52REF.1(HPV52)_1_20
gacctgcaaacacaggaaaatcatattttggaatgagtttaattaggttcttaagtggatgtgtaatatcctatgtaaactcaaaaagccatttttggctacaaccattaacagatgcaa

SEQ ID NO: 267 - DNA, 120 bp, human papillomavirus
  133445_5511603_HPV52REF.1(HPV52)_1_21
aagtgggtatgatagatgatgtaacacctatatgttggacatatatagatgattatatgagaaatgcactggatggaaatgatatatcagtagatgtaaagcatagagccttagtacaaa

SEQ ID NO: 268 - DNA, 120 bp, human papillomavirus
  133445_5511603_HPV52REF.1(HPV52)_1_22
taaaatgcccaccattaattttaacaacaaatacaaatgcaggaacagatcctaggtggccatatttacatagtagattggttgtgtttcatttcaaaaacccatttccatttgatgaaa

SEQ ID NO: 269 - DNA, 120 bp, human papillomavirus
  133445_5511603_HPV52REF.1(HPV52)_1_23
atggcaatcctatatatgaaattaacaacgaaaattggaaatcctttttctcaaggacgtggtgcaaattagatttaatacaggaagaggacaaggaaaacgatggagtcgataccggca

SEQ ID NO: 270 - DNA, 120 bp, human papillomavirus
  133445_5511603_HPV52REF.1(HPV52)_1_24
cgtttaaatgcagtgcaggaaaaaatactagatctatacgaagctgatagtaatgacctaaacgcacaaattgaacattggaaattgactcgaatggaatgtgttttgttttacaaagca

SEQ ID NO: 271 - DNA, 120 bp, human papillomavirus
  133445_5511603_HPV52REF.1(HPV52)_1_25
aaggaactgggaataactcatataggccaccaggtggtgccaccaatggcagtgtctaaggcaaaggcctgccaagctattgaactacaattggcattggaggcattaaacaaaacacaa

SEQ ID NO: 272 - DNA, 120 bp, human papillomavirus
  133445_5511603_HPV52REF.1(HPV52)_1_26
tatagcacagatggatggacattacaacaaacaagtctagaaatgtggcgtgcagaaccacaaaaatactttaaaaaacatgggtatacaataacagtgcaatacgataatgataaaaac

SEQ ID NO: 273 - DNA, 120 bp, human papillomavirus
  133445_5511603_HPV52REF.1(HPV52)_1_27
aatactatggattatacaaactggaaggaaatttatttacttggtgagtgtgaatgtacaattgtagaaggacaagtagattactatgggttatattattggtgtgatggagaaaaaata

SEQ ID NO: 274 - DNA, 120 bp, human papillomavirus
  133445_5511603_HPV52REF.1(HPV52)_1_28
tattttgtaaaatttagtaacgatgcaaagcaatattgtgtaacaggagtatgggaagtacatgtgggtggtcaggtaattgtttgtcctgcatctgtatctagtaacgaagtatccact

SEQ ID NO: 275 - DNA, 120 bp, human papillomavirus
  133445_5511603_HPV52REF.1(HPV52)_1_29
actgaaactgctgtccacctatgcaccgaaacctccaagacctccgcagtgtccgtgggtgccaaagacacacacctacaaccaccacagaaacgacgacgaccagacgtcacagactcc

SEQ ID NO: 276 - DNA, 120 bp, human papillomavirus
  133445_5511603_HPV52REF.1(HPV52)_1_30
agaaacaccaagtaccccaacaaccttttgcggggacaacaatccgtggacagtactacacggggactcgtcactgcaactgagtgcacaaacaaaggacgggttgcacatacaacttgt

SEQ ID NO: 277 - DNA, 120 bp, human papillomavirus
  133445_5511603_HPV52REF.1(HPV52)_1_31
actgcacctataatacacctaaaaggtgatcctaatagtttaaaatgtttaagatatagggtaaaaacacataaaagtttgtatgttcaaatttcatctacctggcattggaccagtaat

SEQ ID NO: 278 - DNA, 120 bp, human papillomavirus
  133445_5511603_HPV52REF.1(HPV52)_1_32
gaatgtacaaataataaactaggtattgtaacaataacgtacagtgatgaaacacaacgtcaacaatttttaaaaactgttaaaataccaaatactgtgcaagttatacaaggtgtcatg

SEQ ID NO: 279 - DNA, 120 bp, human papillomavirus
  133445_5511604_HPV52REF.1(HPV52)_2_1
tatatatgtatatgtgtatggtaaacacccaacacaagccaatattgctgctattgtgtatatataacaatgttaggattatttgtattttgttttattttgcttatggtgttttgtgca

SEQ ID NO: 280 - DNA, 120 bp, human papillomavirus
  133445_5511604_HPV52REF.1(HPV52)_2_2
gtgcttaggccgctcttgctatctatatcggtgtatgcgcaggtgttggtgctggtgcttttgctatgggtatctattgggtcaccatttaaagtgttttttttgtacctactgttttta

SEQ ID NO: 281 - DNA, 120 bp, human papillomavirus
  133445_5511604_HPV52REF.1(HPV52)_2_3
tattttccaatgttttgtattcactgtcatgcacagtatttggcacaactgcaataactgtacatgtagattggctacatgcatatatgcaaaatatactttttcacttttgtagtttgt

SEQ ID NO: 282 - DNA, 120 bp, human papillomavirus
  133445_5511604_HPV52REF.1(HPV52)_2_4
ctaataaatacttttatattttttaatagcttgtcgcaatgagatacagacggtctacacggcacaaacgtgcttctgcaacacagctatatcaaacatgcaaagcctctggcacctgcc

SEQ ID NO: 283 - DNA, 120 bp, human papillomavirus
  133445_5511604_HPV52REF.1(HPV52)_2_5
cccccgatgttattcctaaagtggaaggcacaactattgcagatcaacttttaaaatatggcagcctaggggtgttttttggaggtttgggtataggtacaggtgcaggctctggtggta

SEQ ID NO: 284 - DNA, 120 bp, human papillomavirus
  133445_5511604_HPV52REF.1(HPV52)_2_6
gggcaggctatgtgccattgtccactcgtcctcccactagtagtattaccacgtccaccattcgtccccctgtaactgtagaacccattggtcccttagaaccatctatagtttctatga

SEQ ID NO: 285 - DNA, 120 bp, human papillomavirus
  133445_5511604_HPV52REF.1(HPV52)_2_7
tagaagaaacaacatttattgagtctggcgcacctgctccatctattccatcagcaacagggtttgatgttacaacatctgcaaataatactcctgcaataattaatgtaacatctatag

SEQ ID NO: 286 - DNA, 120 bp, human papillomavirus
  133445_5511604_HPV52REF.1(HPV52)_2_8
gtgaatcatctgtacaatcagtttctacacatttaaatcctacattcactgaaccatctataatacagcccccggcacctgcagaagcatctggtcatgtattgttttctagtccaacta

SEQ ID NO: 287 - DNA, 120 bp, human papillomavirus
  133445_5511604_HPV52REF.1(HPV52)_2_9
ttagtacacacacctatgaagaaatccctatggatacatttgttacctctactgacagcagcagtgtaacaagtagtacacctattccagggtctcgccctacgacacgccttggtttat

SEQ ID NO: 288 - DNA, 120 bp, human papillomavirus
  133445_5511604_HPV52REF.1(HPV52)_2_10
atagccgtgccacacaacaggttaaggtagtcgaccctgcttttatgtcatcaccacagaaattagtaacatataacaatcctgtttttgagggcgttgatacagatgaaactataattt

SEQ ID NO: 289 - DNA, 120 bp, human papillomavirus
  133445_5511604_HPV52REF.1(HPV52)_2_11
ttgatcgttcacaacttttacctgcaccggatcctgattttttagacattatagctttgcataggcctgcattaacctctcgaagaggtactgttaggtttagcaggcttggtaataagg

SEQ ID NO: 290 - DNA, 120 bp, human papillomavirus
  133445_5511604_HPV52REF.1(HPV52)_2_12
ccaccctacgtacacgtagtggaaaacaaattggggcacgggtacattattatcatgatattagtcctatccagcctgctgaagttcaggaagacatagaattgcaacctttattaccac

SEQ ID NO: 291 - DNA, 120 bp, human papillomavirus
  133445_5511604_HPV52REF.1(HPV52)_2_13
agtctgtgtccccttacactattaatgatggtttgtatgatgtgtatgcagattctttgcagcaacccacgtttcacttaccttccacactttctacccataataatactttcactgtac

SEQ ID NO: 292 - DNA, 120 bp, human papillomavirus
  133445_5511604_HPV52REF.1(HPV52)_2_14
ctattaatagtggtattgactttgtatatcaacccactatgtccattgagtcaggtcctgacattccattaccttcgttacccacacatactccttttgttcctatagcccctacagctc

SEQ ID NO: 293 - DNA, 120 bp, human papillomavirus
  133445_5511604_HPV52REF.1(HPV52)_2_15
catctacatctattattgttgatggtacagattttattttacatcctagttattttttactacgtcgcaggcgtaaacgttttccatatttttttacagatgtccgtgtggcggcctagt

SEQ ID NO: 294 - DNA, 120 bp, human papillomavirus
  133445_5511604_HPV52REF.1(HPV52)_2_16
gaggccactgtgtacctgcctcctgtacctgtctctaaggttgtaagcactgatgagtatgtgtctcgcacaagcatctattattatgcaggcagttctcgattactaacagtaggacat

SEQ ID NO: 295 - DNA, 120 bp, human papillomavirus
  133445_5511604_HPV52REF.1(HPV52)_2_17
ccctatttttctattaaaaacaccagtagtggtaatggtaaaaaagttttagttcccaaggtgtctggcctgcaatacagggtatttagaattaaattgccggaccctaataaatttggt

SEQ ID NO: 296 - DNA, 120 bp, human papillomavirus
  133445_5511604_HPV52REF.1(HPV52)_2_18
tttccagatacatctttttataacccagaaacccaaaggttggtgtgggcctgtacaggcttggaaattggtaggggacagcctttaggtgtgggtattagtgggcatcctttattaaac

SEQ ID NO: 297 - DNA, 120 bp, human papillomavirus
  133445_5511604_HPV52REF.1(HPV52)_2_19
aagtttgatgatactgaaaccagtaacaaatatgctggtaaacctggtatagataatagggaatgtttatctatggattataagcagactcagttatgcattttaggatgcaaacctcct

SEQ ID NO: 298 - DNA, 120 bp, human papillomavirus
  133445_5511604_HPV52REF.1(HPV52)_2_20
ataggtgaacattggggtaagggaaccccttgtaataataattcaggaaatcctggggattgtcctcccctacagctcattaacagtgtaatacaggatggggacatggtagatacagga

SEQ ID NO: 299 - DNA, 120 bp, human papillomavirus
  133445_5511604_HPV52REF.1(HPV52)_2_21
tttggttgcatggattttaataccttgcaagctagtaaaagtgatgtgcccattgatatatgtagcagtgtatgtaagtatccagattatttgcaaatggctagcgagccatatggtgac

SEQ ID NO: 300 - DNA, 120 bp, human papillomavirus
  133445_5511604_HPV52REF.1(HPV52)_2_22
agtttgttcttttttcttagacgtgagcaaatgtttgttagacacttttttaatagggccggtaccttaggtgaccctgtgccaggtgatttatatatacaagggtctaactctggcaat

SEQ ID NO: 301 - DNA, 120 bp, human papillomavirus
  133445_5511604_HPV52REF.1(HPV52)_2_23
actgccactgtacaaagcagtgctttttttcctactcctagtggttctatggtaacctcagaatcccaattatttaataaaccgtactggttacaacgtgcgcagggccacaataatggc

SEQ ID NO: 302 - DNA, 120 bp, human papillomavirus
  133445_5511604_HPV52REF.1(HPV52)_2_24
atatgttggggcaatcagttgtttgtcacagttgtggataccactcgtagcactaacatgactttatgtgctgaggttaaaaaggaaagcacatataaaaatgaaaattttaaggaatac

SEQ ID NO: 303 - DNA, 120 bp, human papillomavirus
  133445_5511604_HPV52REF.1(HPV52)_2_25
cttcgtcatggcgaggaatttgatttacaatttatttttcaattgtgcaaaattacattaacagctgatgttatgacatacattcataagatggatgccactattttagaggactggcaa

SEQ ID NO: 304 - DNA, 120 bp, human papillomavirus
  133445_5511604_HPV52REF.1(HPV52)_2_26
tttggccttaccccaccaccgtctgcatctttggaggacacatacagatttgtcacttctactgctataacttgtcaaaaaaacacaccacctaaaggaaaggaagatcctttaaaggac

SEQ ID NO: 305 - DNA, 120 bp, human papillomavirus
  133445_5511604_HPV52REF.1(HPV52)_2_27
tatatgttttgggaggtggatttaaaagaaaagttttctgcagatttagatcagtttcctttaggtaggaagtttttgttacaggcagggctacaggctaggcccaaactaaaacgccct

SEQ ID NO: 306 - DNA, 120 bp, human papillomavirus
  133445_5511604_HPV52REF.1(HPV52)_2_28
gcatcatcggccccacgtacctccacaaagaagaaaaaggttaaaaggtaaccattgtctgttgggtaattgtctgtgtcatgtatgtgttgtgtatgtcaaacacaggttaaaaggtaa

SEQ ID NO: 307 - DNA, 120 bp, human papillomavirus
  133445_5511604_HPV52REF.1(HPV52)_2_29
ccattgtttgttatgtaattgttttgtgtgtgtactgtgttgtttgcatgttatgtatgtgtgtgcatgtttgttgtatttgtcagttcctgtatgtatgttttgtgtatgtattaataa

SEQ ID NO: 308 - DNA, 117 bp, human papillomavirus
  133445_5511604_HPV52REF.1(HPV52)_2_30
agtactgtatttactaaactatttatagtagtcttatgttatgttatggttgcacccacatgagtaacaatacagttgctcctaatctattgcatctcctgccctaccctgtgtccc

SEQ ID NO: 309 - DNA, 120 bp, human papillomavirus
  133445_5511605_HPV52REF.1(HPV52)_3_1
ccctaccctgtgtcctactttgttacactactaattagccttatactctccattttgtaccattttgtactatccaccattttaaatcctaaccgaattcggttggtcttggcacaactt

SEQ ID NO: 310 - DNA, 120 bp, human papillomavirus
  133445_5511605_HPV52REF.1(HPV52)_3_2
tggttgtccttggcacagtaacaactatttttatataagtttcagcaaactgcttaatcctttggtttcctgcagtccactggtctacacttgttgtcccgcctaaactgacttcttgct

SEQ ID NO: 311 - DNA, 120 bp, human papillomavirus
  133445_5511605_HPV52REF.1(HPV52)_3_3
gactcacaggtcctgcagtgcagctaaacaatacattgcctaacattgcatgttttaaactgcttttaggcacatattttatttaaactttcaatgcactaattacagtgttggcttaca

SEQ ID NO: 312 - DNA, 119 bp, human papillomavirus
  133445_5511605_HPV52REF.1(HPV52)_3_4
caagtacatcctacgccaaatatgtcttgtaaaacatgattaaatactgttactcaccaggtgtgcactacacgaccggttacggttaccgtacccacaaccacttttttttataatta

SEQ ID NO: 313 - DNA, 120 bp, human papillomavirus
  88160_3764094_HPV16_1_1
actacaataattcatgtataaaactaagggcgtaaccgaaatcggttgaaccgaaaccggttagtataaaagcagacattttatgcaccaaaagagaactgcaatgtttcaggacccaca

SEQ ID NO: 314 - DNA, 120 bp, human papillomavirus
  88160_3764094_HPV16_1_2
ggagcgacccagaaagttaccacagttatgcacagagctgcaaacaactatacatgatataatattagaatgtgtgtactgcaagcaacagttactgcgacgtgaggtatatgactttgc

SEQ ID NO: 315 - DNA, 120 bp, human papillomavirus
  88160_3764094_HPV16_1_3
ttttcgggatttatgcatagtatatagagatgggaatccatatgctgtatgtgataaatgtttaaagttttattctaaaattagtgagtatagacattattgttatagtttgtatggaac

SEQ ID NO: 316 - DNA, 120 bp, human papillomavirus
  88160_3764094_HPV16_1_4
aacattagaacagcaatacaacaaaccgttgtgtgatttgttaattaggtgtattaactgtcaaaagccactgtgtcctgaagaaaagcaaagacatctggacaaaaagcaaagattcca

SEQ ID NO: 317 - DNA, 120 bp, human papillomavirus
  88160_3764094_HPV16_1_5
taatataaggggtcggtggaccggtcgatgtatgtcttgttgcagatcatcaagaacacgtagagaaacccagctgtaatcatgcatggagatacacctacattgcatgaatatatgtta

SEQ ID NO: 318 - DNA, 120 bp, human papillomavirus
  88160_3764094_HPV16_1_6
gatttgcaaccagagacaactgatctctactgttatgagcaattaaatgacagctcagaggaggaggatgaaatagatggtccagctggacaagcagaaccggacagagcccattacaat

SEQ ID NO: 319 - DNA, 120 bp, human papillomavirus
  88160_3764094_HPV16_1_7
attgtaaccttttgttgcaagtgtgactctacgcttcggttgtgcgtacaaagcacacacgtagacattcgtactttggaagacctgttaatgggcacactaggaattgtgtgccccatc

SEQ ID NO: 320 - DNA, 120 bp, human papillomavirus
  88160_3764094_HPV16_1_8
tgttctcagaaaccataatctaccatggctgatcctgcaggtaccaatggggaagagggtacgggatgtaatggatggttttatgtagaggctgtagtggaaaaaaaaacaggggatgct

SEQ ID NO: 321 - DNA, 120 bp, human papillomavirus
  88160_3764094_HPV16_1_9
atatcagatgacgagaacgaaaatgacagtgatacaggtgaagatttggtagattttatagtaaatgataatgattatttaacacaggcagaaacagagacagcacatgcgttgtttact

SEQ ID NO: 322 - DNA, 120 bp, human papillomavirus
  88160_3764094_HPV16_1_10
gcacaggaagcaaaacaacatagagatgcagtacaggttctaaaacgaaagtatttgggtagtccacttagtgatattagtggatgtgtagacaataatattagtcctagattaaaagct

SEQ ID NO: 323 - DNA, 120 bp, human papillomavirus
  88160_3764094_HPV16_1_11
atatgtatagaaaaacaaagtagagctgcaaaaaggagattatttgaaagcgaagacagcgggtatggcaatactgaagtggaaactcagcagatgttacaggtagaagggcgccatgag

SEQ ID NO: 324 - DNA, 120 bp, human papillomavirus
  88160_3764094_HPV16_1_12
actgaaacaccatgtagtcagtatagtggtggaagtgggggtggttgcagtcagtacagtagtggaagtgggggagagggtgttagtgaaagacacactatatgccaaacaccacttaca

SEQ ID NO: 325 - DNA, 120 bp, human papillomavirus
  88160_3764094_HPV16_1_13
aatattttaaatgtactaaaaactagtaatgcaaaggcagcaatgttagcaaaatttaaagagttatacggggtgagtttttcagaattagtaagaccatttaaaagtaataaatcaacg

SEQ ID NO: 326 - DNA, 120 bp, human papillomavirus
  88160_3764094_HPV16_1_14
tgttgcgattggtgtattgctgcatttggacttacacccagtatagctgacagtataaaaacactattacaacaatattgtttatatttacacattcaaagtttagcatgttcatgggga

SEQ ID NO: 327 - DNA, 120 bp, human papillomavirus
  88160_3764094_HPV16_1_15
atggttgtgttactattagtaagatataaatgtggaaaaaatagagaaacaattgaaaaattgctgtctaaactattatgtgtgtctccaatgtgtatgatgatagagcctccaaaattg

SEQ ID NO: 328 - DNA, 120 bp, human papillomavirus
  88160_3764094_HPV16_1_16
cgtagtacagcagcagcattatattggtataaaacaggtatatcaaatattagtgaagtgtatggagacacgccagaatggatacaaagacaaacagtattacaacatagttttaatgat

SEQ ID NO: 329 - DNA, 120 bp, human papillomavirus
  88160_3764094_HPV16_1_17
tgtacatttgaattatcacagatggtacaatgggcctacgataatgacatagtagacgatagtgaaattgcatataaatatgcacaattggcagacactaatagtaatgcaagtgccttt

SEQ ID NO: 330 - DNA, 120 bp, human papillomavirus
  88160_3764094_HPV16_1_18
ctaaaaagtaattcacaggcaaaaattgtaaaggattgtgcaacaatgtgtagacattataaacgagcagaaaaaaaacaaatgagtatgagtcaatggataaaatatagatgtgatagg

SEQ ID NO: 331 - DNA, 120 bp, human papillomavirus
  88160_3764094_HPV16_1_19
gtagatgatggaggtgattggaagcaaattgttatgtttttaaggtatcaaggtgtagagtttatgtcatttttaactgcattaaaaagatttttgcaaggcatacctaaaaaaaattgc

SEQ ID NO: 332 - DNA, 120 bp, human papillomavirus
  88160_3764094_HPV16_1_20
atattactatatggtgcagctaacacaggtaaatcattatttggtatgagtttaatgaaatttctgcaagggtctgtaatatgttttgtaaattctaaaagccatttttggttacaacca

SEQ ID NO: 333 - DNA, 120 bp, human papillomavirus
  88160_3764094_HPV16_1_21
ttagcagatgccaaaataggtatgttagatgatgctacagtgccctgttggaactacatagatgacaatttaagaaatgcattggatggaaatttagtttctatggatgtaaagcataga

SEQ ID NO: 334 - DNA, 120 bp, human papillomavirus
  88160_3764094_HPV16_1_22
ccattggtacaactaaaatgccctccattattaattacatctaacattaatgctggtacagattctaggtggccttatttacataatagattggtggtgtttacatttcctaatgagttt

SEQ ID NO: 335 - DNA, 120 bp, human papillomavirus
  88160_3764094_HPV16_1_23
ccatttgacgaaaacggaaatccagtgtatgagcttaatgataagaactggaaatcctttttctcaaggacgtggtccagattaagtttgcacgaggacgaggacaaggaaaacgatgga

SEQ ID NO: 336 - DNA, 120 bp, human papillomavirus
  88160_3764094_HPV16_1_24
gactctttgccaacgtttaaatgtgtgtcaggacaaaatactaacacattatgaaaatgatagtacagacctacgtgaccatatagactattggaaacacatgcgcctagaatgtgctat

SEQ ID NO: 337 - DNA, 120 bp, human papillomavirus
  88160_3764094_HPV16_1_25
ttattacaaggccagagaaatgggatttaaacatattaaccaccaggtggtgccaacactggctgtatcaaagaataaagcattacaagcaattgaactgcaactaacgttagaaacaat

SEQ ID NO: 338 - DNA, 120 bp, human papillomavirus
  88160_3764094_HPV16_1_26
atataactcacaatatagtaatgaaaagtggacattacaagacgttagccttgaagtgtatttaactgcaccaacaggatgtataaaaaaacatggatatacagtggaagtgcagtttga

SEQ ID NO: 339 - DNA, 120 bp, human papillomavirus
  88160_3764094_HPV16_1_27
tggagacatatgcaatacaatgcattatacaaactggacacatatatatatttgtgaagaagcatcagtaactgtggtagagggtcaagttgactattatggtttatattatgttcatga

SEQ ID NO: 340 - DNA, 120 bp, human papillomavirus
  88160_3764094_HPV16_1_28
aggaatacgaacatattttgtgcagtttaaagatgatgcagaaaaatatagtaaaaataaagtatgggaagttcatgcgggtggtcaggtaatattatgtcctacatctgtgtttagcag

SEQ ID NO: 341 - DNA, 120 bp, human papillomavirus
  88160_3764094_HPV16_1_29
caacgaagtatcctctcctgaaattattaggcagcacttggccaaccaccccgccgcgacccataccaaagccgtcgccttgggcaccgaagaaacacagacgactatccagcgaccaag

SEQ ID NO: 342 - DNA, 120 bp, human papillomavirus
  88160_3764094_HPV16_1_30
atcagagccagacaccggaaacccctgccacaccactaagttgttgcacagagactcagtggacagtgctccaatcctcactgcatttaacagctcacacaaaggacggattaactgtaa

SEQ ID NO: 343 - DNA, 120 bp, human papillomavirus
  88160_3764094_HPV16_1_31
tagtaacactacacccatagtacatttaaaaggtgatgctaatactttaaaatgtttaagatatagatttaaaaagcattgtacattgtatactgcagtgtcgtctacatggcattggac

SEQ ID NO: 344 - DNA, 120 bp, human papillomavirus
  88160_3764094_HPV16_1_32
aggacataatgtaaaacataaaagtgcaattgttacacttacatatgatagtgaatggcaacgtgaccaatttttgtctcaagttaaaataccaaaaactattacagtgtctactggatt

SEQ ID NO: 345 - DNA, 120 bp, human papillomavirus
  88160_3764094_HPV16_1_33
tatgtctatatgacaaatcttgatactgcatccacaacattactggcgtgctttttgctttgcttttgtgtgcttttgtgtgtctgcctattaatacgtccgctgcttttgtctgtgtct

SEQ ID NO: 346 - DNA, 120 bp, human papillomavirus
  88160_3764094_HPV16_1_34
acatacacatcattaataatattggtattactattgtggataacagcagcctctgcgtttaggtgttttattgtatatattatatttgtttatataccattatttttaatacatacacat

SEQ ID NO: 347 - DNA, 120 bp, human papillomavirus
  88160_3764095_HPV16_2_1
tttttttttttgtttgtttgtttgttttttaataaactgttattacttaacaatgcgacacaaacgttctgcaaaacgcacaaaacgtgcatcggctacccaactttataaaacatgcaa

SEQ ID NO: 348 - DNA, 120 bp, human papillomavirus
  88160_3764095_HPV16_2_2
acaggcaggtacatgtccacctgacattatacctaaggttgaaggcaaaactattgctgatcaaatattacaatatggaagtatgggtgtattttttggtgggttaggaattggaacagg

SEQ ID NO: 349 - DNA, 120 bp, human papillomavirus
  88160_3764095_HPV16_2_3
gtcgggtacaggcggacgcactgggtatattccattgggaacaaggcctcccacagctacagatacacttgctcctgtaagaccccctttaacagtagatcctgtgggcccttctgatcc

SEQ ID NO: 350 - DNA, 120 bp, human papillomavirus
  88160_3764095_HPV16_2_4
ttctatagtttctttagtggaagaaactagttttattgatgctggtgcaccaacatctgtaccttccattcccccagatgtatcaggatttagtattactacttcaactgataccacacc

SEQ ID NO: 351 - DNA, 120 bp, human papillomavirus
  88160_3764096_HPV16_3_1
tattagatattaataatactgttactactgttactacacataataatcccactttcactgacccatctgtattgcagcctccaacacctgcagaaactggagggcattttacactttcat

SEQ ID NO: 352 - DNA, 120 bp, human papillomavirus
  88160_3764096_HPV16_3_2
catccactattagtacacataattatgaagaaattcctatggatacatttattgttagcacaaaccctaacacagtaactagtagcacacccataccagggtctcgcccagtggcacgcc

SEQ ID NO: 353 - DNA, 120 bp, human papillomavirus
  88160_3764096_HPV16_3_3
taggattatatagtcgcacaacacaacaggttaaagttgtagaccctgcttttgtaaccactcccactaaacttattacatatgataatcctgcatatgaaggtatagatgtggataata

SEQ ID NO: 354 - DNA, 120 bp, human papillomavirus
  88160_3764096_HPV16_3_4
cattatatttttctagtaatgataatagtattaatatagctccagatcctgactttttggatatagttgctttacataggccagcattaacctctaggcgtactggcattaggtacagta

SEQ ID NO: 355 - DNA, 120 bp, human papillomavirus
  88160_3764096_HPV16_3_5
gaattggtaataaacaaacactacgtactcgtagtggaaaatctataggtgctaaggtacattattattatgatttaagtactattgatcctgcagaagaaatagaattacaaactataa

SEQ ID NO: 356 - DNA, 120 bp, human papillomavirus
  88160_3764096_HPV16_3_6
caccttctacatatactaccacttcacatgcagcctcacctacttctattaataatggattatatgatatttatgcagatgactttattacagatacttctacaaccccggtaccatctg

SEQ ID NO: 357 - DNA, 120 bp, human papillomavirus
  88160_3764096_HPV16_3_7
taccctctacatctttatcaggttatattcctgcaaatacaacaattccttttggtggtgcatacaatattcctttagtatcaggtcctgatatacccattaatataactgaccaagctc

SEQ ID NO: 358 - DNA, 120 bp, human papillomavirus
  88160_3764096_HPV16_3_8
cttcattaattcctatagttccagggtctccacaatatacaattattgctgatgcaggtgacttttatttacatcctagttattacatgttacgaaaacgacgtaaacgtttaccatatt

SEQ ID NO: 359 - DNA, 120 bp, human papillomavirus
  88160_3764096_HPV16_3_9
ttttttcagatgtctctttggctgcctagtgaggccactgtctacttgcctcctgtcccagtatctaaggttgtaagcacggatgaatatgttgcacgcacaaacatatattatcatgca

SEQ ID NO: 360 - DNA, 120 bp, human papillomavirus
  88160_3764096_HPV16_3_10
ggaacatccagactacttgcagttggacatccctattttcctattaaaaaacctaacaataacaaaatattagttcctaaagtatcaggattacaatacagggtatttagaatacattta

SEQ ID NO: 361 - DNA, 120 bp, human papillomavirus
  88160_3764096_HPV16_3_11
cctgaccccaataagtttggttttcctgacacctcattttataatccagatacacagcggctggtttgggcctgtgtaggtgttgaggtaggtcgtggtcagccattaggtgtgggcatt

SEQ ID NO: 362 - DNA, 120 bp, human papillomavirus
  88160_3764096_HPV16_3_12
agtggccatcctttattaaataaattggatgacacagaaaatgctagtgcttatgcagcaaatgcaggtgtggataatagagaatgtatatctatggattacaaacaaacacaattgtgt

SEQ ID NO: 363 - DNA, 120 bp, human papillomavirus
  88160_3764096_HPV16_3_13
ttaattggttgcaaaccacctataggggaacactggggcaaaggatccccatgtaccaatgttgcagtaaatccaggtgattgtccaccattagagttaataaacacagttattcaggat

SEQ ID NO: 364 - DNA, 120 bp, human papillomavirus
  88160_3764096_HPV16_3_14
ggtgatatggttgatactggctttggtgctatggactttactacattacaggctaacaaaagtgaagttccactggatatttgtacatctatttgcaaatatccagattatattaaaatg

SEQ ID NO: 365 - DNA, 120 bp, human papillomavirus
  88160_3764096_HPV16_3_15
gtgtcagaaccatatggcgacagcttatttttttatttacgaagggaacaaatgtttgttagacatttatttaatagggctggtactgttggtgaaaatgtaccagacgatttatacatt

SEQ ID NO: 366 - DNA, 120 bp, human papillomavirus
  88160_3764096_HPV16_3_16
aaaggctctgggtctactgcaaatttagccagttcaaattattttcctacacctagtggttctatggttacctctgatgcccaaatattcaataaaccttattggttacaacgagcacag

SEQ ID NO: 367 - DNA, 120 bp, human papillomavirus
  88160_3764096_HPV16_3_17
ggccacaataatggcatttgttggggtaaccaactatttgttactgttgttgatactacacgcagtacaaatatgtcattatgtgctgccatatctacttcagaaactacatataaaaat

SEQ ID NO: 368 - DNA, 120 bp, human papillomavirus
  88160_3764096_HPV16_3_18
actaactttaaggagtacctacgacatggggaggaatatgatttacagtttatttttcaactgtgcaaaataaccttaactgcagacgttatgacatacatacattctatgaattccact

SEQ ID NO: 369 - DNA, 120 bp, human papillomavirus
  88160_3764096_HPV16_3_19
attttggaggactggaattttggtctacaacctcccccaggaggcacactagaagatacttataggtttgtaacatcccaggcaattgcttgtcaaaaacatacacctccagcacctaaa

SEQ ID NO: 370 - DNA, 120 bp, human papillomavirus
  88160_3764096_HPV16_3_20
gaagatccccttaaaaaatacactttttgggaagtaaatttaaaggaaaagttttctgcagacctagatcagtttcctttaggacgcaaatttttactacaagcaggattgaaggccaaa

SEQ ID NO: 371 - DNA, 120 bp, human papillomavirus
  88160_3764096_HPV16_3_21
ccaaaatttacattaggaaaacgaaaagctacacccaccacctcatctacctctacaactgctaaacgcaaaaaacgtaagctgtaagtattgtatgtatgttgaattagtgttgtttgt

SEQ ID NO: 372 - DNA, 120 bp, human papillomavirus
  88160_3764097_HPV16_4_2
gtcatgcaacataaataaacttattgtttcaacacctactaattgtgttgtggttattcattgtatataaactatatttgctacatcctgtttttgttttatatatactatattttgtag

SEQ ID NO: 373 - DNA, 120 bp, human papillomavirus
  88160_3764097_HPV16_4_3
cgccagcggccattttgtagcttcaaccgaattcggttgcatgctttttggcacaaaatgtgtttttttaaatagttctatgtcagcaactatggtttaaacttgtacgtttcctgcttg

SEQ ID NO: 374 - DNA, 120 bp, human papillomavirus
  88160_3764097_HPV16_4_4
ccatgcgtgccaaatccctgttttcctgacctgcactgcttgccaaccattccattgttttttacactgcactatgtgcaactactgaatcactatgtacattgtgtcatataaaataaa

SEQ ID NO: 375 - DNA, 120 bp, human papillomavirus
  88160_3764097_HPV16_4_5
tcactatgcgccaacgccttacataccgctgttaggcacatatttttggcttgttttaactaacctaattgcatatttggcataaggtttaaacttctaaggccaactaaatgtcaccct

SEQ ID NO: 376 - DNA, 119 bp, human papillomavirus
  88160_3764097_HPV16_4_6
agttcatacatgaactgtgtaaaggttagtcatacattgttcatttgtaaaactgcacatgggtgtgtgcaaaccgttttgggttacacatttacaagcaacttatataataatactaa

SEQ ID NO: 377 - DNA, 120 bp, human papillomavirus
  133919_5536530_KP212157.1-HPV16-CNA138_1_2-REV
tatcatgtatagttgtttgcagctctgtgcataactgtggtaactttctgggtcgctcctgtgggtcctgaaacattgcagttctcttttggtgcataaaatgtctgcttttatactaac

SEQ ID NO: 378 - DNA, 120 bp, human papillomavirus
  133919_5536530_KP212157.1-HPV16-CNA138_1_4-REV
ttatcacatacagcatatggattcccatctctatatactatgcataaatcccgaaaagcaaagtcatatacctcacgtcgcagtaactgttgcttgcagtacacacattctaatattata

SEQ ID NO: 379 - DNA, 120 bp, human papillomavirus
  133919_5536530_KP212157.1-HPV16-CNA138_1_6-REV
aatacacctaattaacaaatcacacaacggtttgttgtattgctgttctaatgttgttccatacaaactataacaataatgtctatactcactaattttagaataaaactttaaacattt

SEQ ID NO: 380 - DNA, 120 bp, human papillomavirus
  133919_5536530_KP212157.1-HPV16-CNA138_1_8-REV
ttgatgatctgcaacaagacatacatcgaccggtccaccgaccccttatattatggaatctttgctttttgtccagatgtctttgcttttcttcaggacacagtggcttttgacagttaa

SEQ ID NO: 381 - DNA, 120 bp, human papillomavirus
  133919_5536530_KP212157.1-HPV16-CNA138_1_10-REV
gtcatttaattgctcataacagtagagatcagttgtctctggttgcaaatctaacatatattcatgcaatgtaggtgtatctccatgcatgattacagctgggtttctctacgtgttctt

SEQ ID NO: 382 - DNA, 120 bp, human papillomavirus
  133919_5536530_KP212157.1-HPV16-CNA138_1_12-REV
gtacgcacaaccgaagcgtagagtcacacttgcaacaaaaggttacaatattgtaatgggctctgtccggttctgcttgtccagctggaccatctatttcatcctcctcctctgagctgt

SEQ ID NO: 383 - DNA, 120 bp, human papillomavirus
  133919_5536530_KP212157.1-HPV16-CNA138_1_14-REV
ttggtacctgcaggatcagccatggtagattatggtttctgagaacagatggggcacacaattcctagtgtgcccattaacaggtcttccaaagtacgaatgtctacgtgtgtgctttgt

SEQ ID NO: 384 - DNA, 120 bp, human papillomavirus
  133919_5536530_KP212157.1-HPV16-CNA138_1_16-REV
atcttcacctgtatcactgtcattttcgttctcgtcatctgatatagcatcccctgtttttttttccactacagcctctacataaaaccatccattacatcccgtaccctcttccccatt

SEQ ID NO: 385 - DNA, 120 bp, human papillomavirus
  133919_5536530_KP212157.1-HPV16-CNA138_1_18-REV
ttagaacctgtactgcatctctatgttgttttgcttcctgtgcagtaaacaacgcatgtgctgtctctgtttctgcctgtgttaaataatcattatcatttactataaaatctaccaaat

SEQ ID NO: 386 - DNA, 120 bp, human papillomavirus
  133919_5536530_KP212157.1-HPV16-CNA138_1_20-REV
aatctcctttttgcagctctactttgtttttctatacatatagcttttaatctaggactaatattattgtctacacatccactaatatcactaagtggactacccaaatactttcgtttt

SEQ ID NO: 387 - DNA, 120 bp, human papillomavirus
  133919_5536530_KP212157.1-HPV16-CNA138_1_22-REV
cccacttccaccactatactgactacatggtgtttcagtctcatggcgcccttctacctgtaacatctgctgagtttccacttcagtattgccatacccgctgtcttcgctttcaaataa

SEQ ID NO: 388 - DNA, 120 bp, human papillomavirus
  133919_5536530_KP212157.1-HPV16-CNA138_1_24-REV
cctttgcattactagtttttagtacatttaaaatatttgtaagtggtgtttggcatatagtgtgtctttcactaacaccctctcccccacttccactactgtactgactgcaaccacccc

SEQ ID NO: 389 - DNA, 120 bp, human papillomavirus
  133919_5536530_KP212157.1-HPV16-CNA138_1_26-REV
gtaagtccaaatgcagcaatacaccaatcgcaacacgttgatttattacttttaaatggtcttactaattctgaaaaactcaccccgtataactctttaaattttgctaacattgctgcc

SEQ ID NO: 390 - DNA, 120 bp, human papillomavirus
  133919_5536530_KP212157.1-HPV16-CNA138_1_28-REV
acatttatatcttactaatagtaacacaaccattccccatgaacatgctaaactttgaatgtgtaaatataaacaatattgttgtaatagtgtttttatactgtcagctatactgggtgt

SEQ ID NO: 391 - DNA, 120 bp, human papillomavirus
  133919_5536530_KP212157.1-HPV16-CNA138_1_30-REV
tataccaatataatgctgctgctgtactacgcaattttggaggctctatcatcatacacattggagacacacataatagtttagacagcaatttttcaattgtttctctattttttccac

SEQ ID NO: 392 - DNA, 120 bp, human papillomavirus
  133919_5536530_KP212157.1-HPV16-CNA138_1_32-REV
tgtaccatctgtgataattcaaatgtacaatcattaaaactatgttgtaatactgtttgtctttgtatccattctggcgtgtctccatacacttcactaatatttgatatacctgtttta

SEQ ID NO: 393 - DNA, 120 bp, human papillomavirus
  133919_5536530_KP212157.1-HPV16-CNA138_1_34-REV
aatttttgcctgtgaattactttttagaaaggcacttgcattactattagtgtctgccaattgtgcatatttatatgcaatttcactatcgtctactatgtcattatcgtaggcccattg

SEQ ID NO: 394 - DNA, 120 bp, human papillomavirus
  133919_5536530_KP212157.1-HPV16-CNA138_1_36-REV
gcttccaatcacctccatcatctaccctatcacatctatattttatccattgactcatactcatttgttttttttctgctcgtttataatgtctacacattgttgcacaatcctttacaa

SEQ ID NO: 395 - DNA, 120 bp, human papillomavirus
  133919_5536530_KP212157.1-HPV16-CNA138_1_38-REV
ttagctgcaccatatagtaatatgcaattttttttaggtatgccttgcaaaaatctttttaatgcagttaaaaatgacataaactctacaccttgataccttaaaaacataacaatttgc

SEQ ID NO: 396 - DNA, 120 bp, human papillomavirus
  133919_5536530_KP212157.1-HPV16-CNA138_1_40-REV
acctattttggcatctgctaatggttgtaaccaaaaatggcttttagaatttacaaaacatattacagacccttgcagaaatttcattaaactcataccaaataatgatttacctgtgtt

SEQ ID NO: 397 - DNA, 120 bp, human papillomavirus
  133919_5536530_KP212157.1-HPV16-CNA138_1_42-REV
attttagttgtaccaatggtctatgctttacatccatagaaactaaatttccatccaatgcatttcttaaattgtcatctatgtagttccaacagggcactgtagcatcatctaacatac

SEQ ID NO: 398 - DNA, 120 bp, human papillomavirus
  133919_5536530_KP212157.1-HPV16-CNA138_1_44-REV
ccgttttcgtcaaatggaaactcattaggaaatgtaaacaccaccaatctattatgtaaataaggccacctagaatctgtaccagcattaatgttagatgtaattaataatggagggcat

SEQ ID NO: 399 - DNA, 120 bp, human papillomavirus
  133919_5536530_KP212157.1-HPV16-CNA138_1_46-REV
cgttggcaaagagtctccatcgttttccttgtcctcgtcctcgtgcaaacttaatctggaccacgtccttgagaaaaaggatttccagttcttatcattaagctcatacactggatttcc

SEQ ID NO: 400 - DNA, 120 bp, human papillomavirus
  133919_5536530_KP212157.1-HPV16-CNA138_1_48-REV
ggccttgtaataaatagcacattctaggcgcatgtgtttccaatagtctatatggtcacgtaggtctgtactatcattttcataatgtgttagtattttgtcctgacacacatttaaacg

SEQ ID NO: 401 - DNA, 120 bp, human papillomavirus
  133919_5536530_KP212157.1-HPV16-CNA138_1_50-REV
gtgagttatatattgtttctaacgttagttgcagttcaattgcttgtaatgctttattctttgatacagccagtgttggcaccacctggtggttaatatgtttaaatcccatttctctgg

SEQ ID NO: 402 - DNA, 120 bp, human papillomavirus
  133919_5536530_KP212157.1-HPV16-CNA138_1_52-REV
atgtctccatcaaactgcacttccactgtatatccatgtttttttatacatcctgttggtgcagttaaatacacttcaaggctaacgtcttgtaatgtccacttttcattactatattgt

SEQ ID NO: 403 - DNA, 120 bp, human papillomavirus
  133919_5536530_KP212157.1-HPV16-CNA138_1_54-REV
tattccttcatgaacataatataaaccataatagtcaacttgaccctctaccacagttactgatgcttcttcacaaatatatatatgtgtccagtttgtataatgcattgtattgcatat

SEQ ID NO: 404 - DNA, 120 bp, human papillomavirus
  133919_5536530_KP212157.1-HPV16-CNA138_1_56-REV
cgttgctgctaaacacagatgtaggacataatattacctgaccacccgcatgaacttcccatactttatttttactatatttttctgcatcatctttaaactgcacaaaatatgttcgta

SEQ ID NO: 405 - DNA, 120 bp, human papillomavirus
  133919_5536530_KP212157.1-HPV16-CNA138_1_58-REV
gatcttggtcgctggatagtcgtctgtgtttcttcggtgcccaaggcgacggctttggtatgggtcgcggcggggtggttggccaagtgctgcctaataatttcaggagaggatacttcg

SEQ ID NO: 406 - DNA, 120 bp, human papillomavirus
  133919_5536530_KP212157.1-HPV16-CNA138_1_60-REV
attacagttaatccgtcctttgtgtgagctgttaaatgcagtgaggattggagcactgtccactgagtctctgtgcaacaacttagtggtgtggcaggggtttccggtgtctggctctga

SEQ ID NO: 407 - DNA, 120 bp, human papillomavirus
  133919_5536530_KP212157.1-HPV16-CNA138_1_62-REV
tccaatgccatgtagacgacactgcagtatacaatgtacaatgctttttaaatctatatcttaaacattttaaagtattagcatcaccttttaaatgtactatgggtgtagtgttactat

SEQ ID NO: 408 - DNA, 120 bp, human papillomavirus
  133919_5536530_KP212157.1-HPV16-CNA138_1_64-REV
ccagtagacactgtaatagtttttggtattttaacttgagacaaaaattggtcacgttgccattcactatcatatgtaagtgtaacaattgcacttttatgttttacattatgtcctgtc

SEQ ID NO: 409 - DNA, 120 bp, human papillomavirus
  133919_5536530_KP212157.1-HPV16-CNA138_1_66-REV
cagacaaaagcagcggacgtattaataggcagacacacaaaagcacacaaaagcaaagcaaaaagcacgccagtaatgttgtggatgcagtatcaagatttgtcatatagacataaatcc

SEQ ID NO: 410 - DNA, 120 bp, human papillomavirus
  133919_5536530_KP212157.1-HPV16-CNA138_1_68-REV
tgtattaaaaataatggtatataaacaaatataatatatacaataaaacacctaaacgcagaggctgctgttatccacaatagtaataccaatattattaatgatgtgtatgtagacaca

SEQ ID NO: 411 - DNA, 86 bp, human papillomavirus
  133919_5536530_KP212157.1-HPV16-CNA138_1_70-REV
tagtaagttatggtatacaacaattatatgtaacaattacattatgtacatatacattatgtaattaaaaagcgtgcatgtgtatg

SEQ ID NO: 412 - DNA, 120 bp, human papillomavirus
  133919_5536531_KP212157.1-HPV16-CNA138_2_2-REV
ggtataatgtcaggtggacatgtacctgcctgtttgcatgttttataaagttgggtagccgatgcacgttttgtgcgttttgcagaacgtttgtgtcgcattgttaagtgataacagttt

SEQ ID NO: 413 - DNA, 120 bp, human papillomavirus
  133919_5536531_KP212157.1-HPV16-CNA138_2_4-REV
ggaatatacccagtgcgtccgcctgtacccgaccccgttccaattcctaacccaccaaaaaatacacccatacttccatattgtaatatttgatcagcaatagttttgccttcaacctta

SEQ ID NO: 414 - DNA, 120 bp, human papillomavirus
  133919_5536531_KP212157.1-HPV16-CNA138_2_6-REV
aaactagtttcttccactaaagaaactatagaaggatcagaagggcccacaggatctactgttaaagggggtcttacaggagcaagtgtatctgtagctgtgggaggccttgttcccaat

SEQ ID NO: 415 - DNA, 120 bp, human papillomavirus
  133919_5536532_KP212157.1-HPV16-CNA138_3_2-REV
ggaatttcttcataattatgtgtactaatagtggatgatgaaagtgtaaaatgccctccagtttctgcaggtgttggaggctgcaatacagatgggtcagtgaaagtgggattattatgt

SEQ ID NO: 416 - DNA, 120 bp, human papillomavirus
  133919_5536532_KP212157.1-HPV16-CNA138_3_4-REV
actttaacttgttgtgttgtgcgactatataatcctaggcgtgccactgggcgagaccctggtatgggtgtgctactagttactgtgttagggtttgtgctaacaataaatgtatccata

SEQ ID NO: 417 - DNA, 120 bp, human papillomavirus
  133919_5536532_KP212157.1-HPV16-CNA138_3_6-REV
atattaatactattatcattactagaaaaatataatgtattatccacatctataccttcatatgcaggattatcatatgtaataagtttagtgggagtggttacaaaagcagggtctaca

SEQ ID NO: 418 - DNA, 120 bp, human papillomavirus
  133919_5536532_KP212157.1-HPV16-CNA138_3_8-REV
ccactacgagtacgtagtgtttgtttattaccaattctactgtacctaatgccagtacgcctagaggttaatgctggcctatgtaaagcaactatatccaaaaagtcaggatctggagct

SEQ ID NO: 419 - DNA, 120 bp, human papillomavirus
  133919_5536532_KP212157.1-HPV16-CNA138_3_10-REV
gaggctgcatgtgaagtggtagtatatgtagaaggtgttatagtttgtaattctatttcttctgcaggatcaatagtactaaaatcataataataatgtaccttagcacctatagatttt

SEQ ID NO: 420 - DNA, 120 bp, human papillomavirus
  133919_5536532_KP212157.1-HPV16-CNA138_3_12-REV
tttgcaggaatataacctgataaagatgtagagggtacagatggtaccggggttgtagaagtatctgtaataaagtcatctgcataaatatcatataatccattattaatagaagtaggt

SEQ ID NO: 421 - DNA, 120 bp, human papillomavirus
  133919_5536532_KP212157.1-HPV16-CNA138_3_14-REV
tattgtggagaccctggaactataggaattaatgaaggagcttggtcagttatattaatgggtatatcaggacctgatactaaaggaatattgtatgcaccaccaaaaggaattgttgta

SEQ ID NO: 422 - DNA, 120 bp, human papillomavirus
  133919_5536532_KP212157.1-HPV16-CNA138_3_16-REV
tggcctcactaggcagccaaagagacatctgaaaaaaaatatggtaaacgtttacgtcgttttcgtaacatgtaataactaggatgtaaataaaagtcacctgcatcagcaataattgta

SEQ ID NO: 423 - DNA, 120 bp, human papillomavirus
  133919_5536532_KP212157.1-HPV16-CNA138_3_18-REV
aatagggatgtccaactgcaagtagtctggatgttcctgcatgataatatatgtttgtgcgtgcaacatattcatccgtgcttacaaccttagatactgggacaggaggcaagtagacag

SEQ ID NO: 424 - DNA, 120 bp, human papillomavirus
  133919_5536532_KP212157.1-HPV16-CNA138_3_20-REV
atgaggtgtcaggaaaaccaaacttattggggtcaggtaaatgtattctaaataccctgtattgtaatcctgatactttaggaactaatattttgttattgttaggttttttaataggaa

SEQ ID NO: 425 - DNA, 120 bp, human papillomavirus
  133919_5536532_KP212157.1-HPV16-CNA138_3_22-REV
ctgtgtcatccaatttatttaataaaggatggccactaatgcccacacctaatggctgaccacgacctacctcaacacctacacaggcccaaaccagccgctgtgtatctggattataaa

SEQ ID NO: 426 - DNA, 120 bp, human papillomavirus
  133919_5536532_KP212157.1-HPV16-CNA138_3_24-REV
cccagtgttcccctataggtggtttgcaaccaattaaacacaattgtgtttgtttgtaatccatagatatacattctctattatccacacctgcatttgctgcataagcactagcatttt

SEQ ID NO: 427 - DNA, 120 bp, human papillomavirus
  133919_5536532_KP212157.1-HPV16-CNA138_3_26-REV
agtccatagcaccaaagccagtatcaaccatatcaccatcctgaataactgtgtttattaactctaatggtggacaatcacctggatttactgcaacattggtacatggggatcctttgc

SEQ ID NO: 428 - DNA, 120 bp, human papillomavirus
  133919_5536532_KP212157.1-HPV16-CNA138_3_28-REV
aataaaaaaataagctgtcgccatatggttctgacaccattttaatataatctggatatttgcaaatagatgtacaaatatccagtggaacttcacttttgttagcctgtaatgtagtaa

SEQ ID NO: 429 - DNA, 120 bp, human papillomavirus
  133919_5536532_KP212157.1-HPV16-CNA138_3_30-REV
tggaactggctaaatttgcagtagacccagagcctttaatgtataaatcgtctggtacattttcaccaacagtaccagccctattaaataaatgtctaacaaacatttgttcccttcgta

SEQ ID NO: 430 - DNA, 120 bp, human papillomavirus
  133919_5536532_KP212157.1-HPV16-CNA138_3_32-REV
atagttggttaccccaacaaatgccattattgtggccctgtgctcgttgtaaccaataaggtttattgaatatttgggcatcagaggtaaccatagaaccactaggtgtaggaaaataat

SEQ ID NO: 431 - DNA, 120 bp, human papillomavirus
  133919_5536532_KP212157.1-HPV16-CNA138_3_34-REV
attcctccccatgtcgtaggtactccttaaagttagtatttttatatgtagtttctgaagtagatatggcagcacataatgacatatttgtactgcgtgtagtatcaacaacagtaacaa

SEQ ID NO: 432 - DNA, 120 bp, human papillomavirus
  133919_5536532_KP212157.1-HPV16-CNA138_3_36-REV
ggggaggttgtagaccaaaattccagtcctccaaaatagtggaattcatagaatgtatgtatgtcataacgtctgcagttaaggttattttgcacagttgaaaaataaactgtaaatcat

SEQ ID NO: 433 - DNA, 120 bp, human papillomavirus
  133919_5536532_KP212157.1-HPV16-CNA138_3_38-REV
ttacttcccaaaaagtgtattttttaaggggatcttctttaggtgctggaggtgtatgtttttgacaagcaattgcctgggatgttacaaacctataagtatcttctagtgtgcctcctg

SEQ ID NO: 434 - DNA, 120 bp, human papillomavirus
  133919_5536532_KP212157.1-HPV16-CNA138_3_40-REV
tgggtgtagcttttcgttttcctaatgtaaattttggtttggccttcaatcctgcttgtagtaaaaatttgcgtcctaaaggaaactgatctaggtctgcagaaaacttttcctttaaat

SEQ ID NO: 435 - DNA, 114 bp, human papillomavirus
  133919_5536533_KP212157.1-HPV16-CNA138_4_2-REV
gatgtagcaaatatagtttatatacaatgaataaccacaacacaattagtaggtgttgaaacaataagtttatttatgttgcatgacacaatagttacacaagcatttaaaaac

SEQ ID NO: 436 - DNA, 120 bp, human papillomavirus
  133919_5536533_KP212157.1-HPV16-CNA138_4_4-REV
ctatttaaaaaaacacattttgtgccaaaaagcttgcaaccgaattcggttgaagctacaaaatggccgctggcgctacaaaatatagtatatataaaacaaaaacaggatgtagcaaat

SEQ ID NO: 437 - DNA, 120 bp, human papillomavirus
  133919_5536533_KP212157.1-HPV16-CNA138_4_6-REV
aaaaaacaatggaatggttggcaagcagtgcaggtcaggaaaacagggatttggcacgcatggcaagcaggaaacgtacaagtttaaaccatagttgctgacatagaactatttaaaaaa

SEQ ID NO: 438 - DNA, 120 bp, human papillomavirus
  133919_5536533_KP212157.1-HPV16-CNA138_4_8-REV
agccaaaaatatgtgcctaacagcggtatgtaaggcgttggcgcatagtgatttattttatatgacacaatgtacatagtgattcagtagttgcacatagtgcagtgtaaaaaacaatgg

SEQ ID NO: 439 - DNA, 120 bp, human papillomavirus
  133919_5536533_KP212157.1-HPV16-CNA138_4_10-REV
caatgtatgactaacctttacacagttcatgtataaactagggtgacatttagttggccttagaagtttaaaccttatgccaaatatgcaattaggttagttaaaacaagccaaaaatat

SEQ ID NO: 440 - DNA, 120 bp, human papillomavirus
  625339_25353837_60975_HPV18REF.1_1_1
cctcaaagcgcgccatagtattgtggtgtgtttctcacatcttttatatacaccgttttcggtcccgaccgttttcggttactcccttttttatatactacaattgttaaaagtattaat

SEQ ID NO: 441 - DNA, 120 bp, human papillomavirus
  625339_25353837_60975_HPV18REF.1_1_2
gaaaacggtgtatataaaagatgtgagaaacacaccacaatactatggcgcgctttgaggatccaacacggcgaccctacaagctacctgatctgtgcacggaactgaacacttcactgc

SEQ ID NO: 442 - DNA, 120 bp, human papillomavirus
  625339_25353837_60975_HPV18REF.1_1_3
caaatacctctgtaagttccaatactgtcttgcaatatacacaggttatttctatgtcttgcagtgaagtgttcagttccgtgcacagatcaggtagcttgtagggtcgccgtgttggat

SEQ ID NO: 443 - DNA, 120 bp, human papillomavirus
  625339_25353837_60975_HPV18REF.1_1_4
aagacatagaaataacctgtgtatattgcaagacagtattggaacttacagaggtatttgaatttgcatttaaagatttatttgtggtgtatagagacagtataccgcatgctgcatgcc

SEQ ID NO: 444 - DNA, 120 bp, human papillomavirus
  625339_25353837_60975_HPV18REF.1_1_5
acacagagtctgaataatgtcttaattctctaattctagaataaaaatctatacatttatggcatgcagcatgcggtatactgtctctatacaccacaaataaatctttaaatgcaaatt

SEQ ID NO: 445 - DNA, 120 bp, human papillomavirus
  625339_25353837_60975_HPV18REF.1_1_6
ataaatgtatagatttttattctagaattagagaattaagacattattcagactctgtgtatggagacacattggaaaaactaactaacactgggttatacaatttattaataaggtgcc

SEQ ID NO: 446 - DNA, 120 bp, human papillomavirus
  625339_25353837_60975_HPV18REF.1_1_7
gtcgtttttcattaaggtgtctaagtttttctgctggattcaacggtttctggcaccgcaggcaccttattaataaattgtataacccagtgttagttagtttttccaatgtgtctccat

SEQ ID NO: 447 - DNA, 120 bp, human papillomavirus
  625339_25353837_60975_HPV18REF.1_1_8
tgcggtgccagaaaccgttgaatccagcagaaaaacttagacaccttaatgaaaaacgacgatttcacaacatagctgggcactatagaggccagtgccattcgtgctgcaaccgagcac

SEQ ID NO: 448 - DNA, 120 bp, human papillomavirus
  625339_25353837_60975_HPV18REF.1_1_9
ggtccatgcatacttaatattatacttgtgtttctctgcgtcgttggagtcgttcctgtcgtgctcggttgcagcacgaatggcactggcctctatagtgcccagctatgttgtgaaatc

SEQ ID NO: 449 - DNA, 120 bp, human papillomavirus
  625339_25353837_60975_HPV18REF.1_1_10
gacaggaacgactccaacgacgcagagaaacacaagtataatattaagtatgcatggacctaaggcaacattgcaagacattgtattgcatttagagccccaaaatgaaattccggttga

SEQ ID NO: 450 - DNA, 120 bp, human papillomavirus
  625339_25353837_60975_HPV18REF.1_1_11
actccatctatttcatcgttttcttcctctgagtcgcttaattgctcgtgacatagaaggtcaaccggaatttcattttggggctctaaatgcaatacaatgtcttgcaatgttgcctta

SEQ ID NO: 451 - DNA, 120 bp, human papillomavirus
  625339_25353837_60975_HPV18REF.1_1_12
ccttctatgtcacgagcaattaagcgactcagaggaagaaaacgatgaaatagatggagttaatcatcaacatttaccagcccgacgagccgaaccacaacgtcacacaatgttgtgtat

SEQ ID NO: 452 - DNA, 120 bp, human papillomavirus
  625339_25353837_60975_HPV18REF.1_1_13
cgaaggtcgtctgctgagctttctactactagctcaattctggcttcacacttacaacacatacacaacattgtgtgacgttgtggttcggctcgtcgggctggtaaatgttgatgatta

SEQ ID NO: 453 - DNA, 120 bp, human papillomavirus
  625339_25353837_60975_HPV18REF.1_1_14
gtgttgtaagtgtgaagccagaattgagctagtagtagaaagctcagcagacgaccttcgagcattccagcagctgtttctgaacaccctgtcctttgtgtgtccgtggtgtgcatccca

SEQ ID NO: 454 - DNA, 120 bp, human papillomavirus
  625339_25353837_60975_HPV18REF.1_1_15
ccgttacaacccgtgccctccccgtctgtaccttctggatcagccattgttgcttactgctgggatgcacaccacggacacacaaaggacagggtgttcagaaacagctgctggaatgct

SEQ ID NO: 455 - DNA, 120 bp, human papillomavirus
  625339_25353837_60975_HPV18REF.1_1_16
gcagtaagcaacaatggctgatccagaaggtacagacggggagggcacgggttgtaacggctggttttatgtacaagctattgtagacaaaaaaacaggagatgtaatatcagatgacga

SEQ ID NO: 456 - DNA, 120 bp, human papillomavirus
  625339_25353837_60975_HPV18REF.1_1_17
gttccttgtgtatcaataaaatctaccatatccgaccctgtgtctgttgcattttcgtcctcgtcatctgatattacatctcctgtttttttgtctacaatagcttgtacataaaaccag

SEQ ID NO: 457 - DNA, 120 bp, human papillomavirus
  625339_25353837_60975_HPV18REF.1_1_18
ggacgaaaatgcaacagacacagggtcggatatggtagattttattgatacacaaggaacattttgtgaacaggcagagctagagacagcacaggcattgttccatgcgcaggaggtcca

SEQ ID NO: 458 - DNA, 120 bp, human papillomavirus
  625339_25353837_60975_HPV18REF.1_1_19
ttttctgtgctgcctcctgcaaactttcgttttaaaacatgcaacacttgtgcatcattgtggacctcctgcgcatggaacaatgcctgtgctgtctctagctctgcctgttcacaaaat

SEQ ID NO: 459 - DNA, 120 bp, human papillomavirus
  625339_25353837_60975_HPV18REF.1_1_20
caatgatgcacaagtgttgcatgttttaaaacgaaagtttgcaggaggcagcacagaaaacagtccattaggggagcggctggaggtggatacagagttaagtccacggttacaagaaat

SEQ ID NO: 460 - DNA, 120 bp, human papillomavirus
  625339_25353837_60975_HPV18REF.1_1_21
ccactatctgatattgtaaacagccgcctttttgcctttttctgcccactatttaaagatatttcttgtaaccgtggacttaactctgtatccacctccagccgctcccctaatggactg

SEQ ID NO: 461 - DNA, 120 bp, human papillomavirus
  625339_25353837_60975_HPV18REF.1_1_22
atctttaaatagtgggcagaaaaaggcaaaaaggcggctgtttacaatatcagatagtggctatggctgttctgaagtggaagcaacacagattcaggtaactacaaatggcgaacatgg

SEQ ID NO: 462 - DNA, 120 bp, human papillomavirus
  625339_25353837_60975_HPV18REF.1_1_23
ttgccctctgtgcccccgttgtctatagcctccgtactgccgccactacatacattgccgccatgttcgccatttgtagttacctgaatctgtgttgcttccacttcagaacagccatag

SEQ ID NO: 463 - DNA, 120 bp, human papillomavirus
  625339_25353837_60975_HPV18REF.1_1_24
cggcaatgtatgtagtggcggcagtacggaggctatagacaacgggggcacagagggcaacaacagcagtgtagacggtacaagtgacaatagcaatatagaaaatgtaaatccacaatg

SEQ ID NO: 464 - DNA, 120 bp, human papillomavirus
  625339_25353837_60975_HPV18REF.1_1_25
gctaacatagctccttgtttattgtttacttttaacaagtcttttaattgtgctatggtacattgtggatttacattttctatattgctattgtcacttgtaccgtctacactgctgttg

SEQ ID NO: 465 - DNA, 120 bp, human papillomavirus
  625339_25353837_60975_HPV18REF.1_1_26
taccatagcacaattaaaagacttgttaaaagtaaacaataaacaaggagctatgttagcagtatttaaagacacatatgggctatcatttacagatttagttagaaattttaaaagtga

SEQ ID NO: 466 - DNA, 120 bp, human papillomavirus
  625339_25353837_60975_HPV18REF.1_1_27
tctgctattgttgggtttactccaaatatagctgtaacccaatctgtacacgtggttttatcacttttaaaatttctaactaaatctgtaaatgatagcccatatgtgtctttaaatact

SEQ ID NO: 467 - DNA, 120 bp, human papillomavirus
  625339_25353837_60975_HPV18REF.1_1_28
taaaaccacgtgtacagattgggttacagctatatttggagtaaacccaacaatagcagaaggatttaaaacactaatacagccatttatattatatgcccatattcaatgtctagactg

SEQ ID NO: 468 - DNA, 120 bp, human papillomavirus
  625339_25353837_60975_HPV18REF.1_1_29
gttagtctactcttaccacatttgtaacgcaacagggctaatattaatactccccatttacagtctagacattgaatatgggcatataatataaatggctgtattagtgttttaaatcct

SEQ ID NO: 469 - DNA, 120 bp, human papillomavirus
  625339_25353837_60975_HPV18REF.1_1_30
taaatggggagtattaatattagccctgttgcgttacaaatgtggtaagagtagactaacagttgctaaaggtttaagtacgttgttacacgtacctgaaacttgtatgttaattcaacc

SEQ ID NO: 470 - DNA, 120 bp, human papillomavirus
  625339_25353837_60975_HPV18REF.1_1_31
atatttgatattcctgttctataccaatatagtgctgcaacactacttcgcaattttggtggttgaattaacatacaagtttcaggtacgtgtaacaacgtacttaaacctttagcaact

SEQ ID NO: 471 - DNA, 120 bp, human papillomavirus
  625339_25353837_60975_HPV18REF.1_1_32
accaaaattgcgaagtagtgttgcagcactatattggtatagaacaggaatatcaaatattagtgaagtaatgggagacacacctgagtggatacaaagacttactattatacaacatgg

SEQ ID NO: 472 - DNA, 120 bp, human papillomavirus
  625339_25353837_60975_HPV18REF.1_1_33
agctcattatcaaatgcccattgtaccatttctgacaaatcaaaattgctatcatctattccatgttgtataatagtaagtctttgtatccactcaggtgtgtctcccattacttcacta

SEQ ID NO: 473 - DNA, 120 bp, human papillomavirus
  625339_25353837_60975_HPV18REF.1_1_34
aatagatgatagcaattttgatttgtcagaaatggtacaatgggcatttgataatgagctgacagatgaaagcgatatggcatttgaatatgccttattagcagacagcaacagcaatgc

SEQ ID NO: 474 - DNA, 120 bp, human papillomavirus
  625339_25353837_60975_HPV18REF.1_1_35
cacattgtggcacaatcttttaaatatttagcttggcaattgctttttaaaaaggcagctgcattgctgttgctgtctgctaataaggcatattcaaatgccatatcgctttcatctgtc

SEQ ID NO: 475 - DNA, 120 bp, human papillomavirus
  625339_25353837_60975_HPV18REF.1_1_36
agctgcctttttaaaaagcaattgccaagctaaatatttaaaagattgtgccacaatgtgcaaacattataggcgagcccaaaaacgacaaatgaatatgtcacagtggatacgatttag

SEQ ID NO: 476 - DNA, 120 bp, human papillomavirus
  625339_25353837_60975_HPV18REF.1_1_37
tggtatcgcaggaattgcactattggtctccaatctcccccttcatctatttttgaacatctaaatcgtatccactgtgacatattcatttgtcgtttttgggctcgcctataatgtttg

SEQ ID NO: 477 - DNA, 120 bp, human papillomavirus
  625339_25353837_60975_HPV18REF.1_1_38
atgttcaaaaatagatgaagggggagattggagaccaatagtgcaattcctgcgataccaacaaatagagtttataacatttttaggagccttaaaatcatttttaaaaggaacccccaa

SEQ ID NO: 478 - DNA, 120 bp, human papillomavirus
  625339_25353837_60975_HPV18REF.1_1_39
ctcattccaaaatatgattttcctgtatttgctggtccacaaaatactaaacaattttttttgggggttccttttaaaaatgattttaaggctcctaaaaatgttataaactctatttgt

SEQ ID NO: 479 - DNA, 120 bp, human papillomavirus
  625339_25353837_60975_HPV18REF.1_1_40
aaaaaattgtttagtattttgtggaccagcaaatacaggaaaatcatattttggaatgagttttatacactttatacaaggagcagtaatatcatttgtgaattccactagtcatttttg

SEQ ID NO: 480 - DNA, 120 bp, human papillomavirus
  625339_25353837_60975_HPV18REF.1_1_41
caacacgtggtcgttgcatcatctaacatggccaccttagtatctgttaacggttccaaccaaaaatgactagtggaattcacaaatgatattactgctccttgtataaagtgtataaaa

SEQ ID NO: 481 - DNA, 120 bp, human papillomavirus
  625339_25353837_60975_HPV18REF.1_1_42
gttggaaccgttaacagatactaaggtggccatgttagatgatgcaacgaccacgtgttggacatactttgatacctatatgagaaatgcgttagatggcaatccaataagtattgatag

SEQ ID NO: 482 - DNA, 120 bp, human papillomavirus
  625339_25353837_60975_HPV18REF.1_1_43
tgtatatttgtggttagtagtattggaggacattttagttgtattaatggtttgtgctttctatcaatacttattggattgccatctaacgcatttctcatataggtatcaaagtatgtc

SEQ ID NO: 483 - DNA, 120 bp, human papillomavirus
  625339_25353837_60975_HPV18REF.1_1_44
aaagcacaaaccattaatacaactaaaatgtcctccaatactactaaccacaaatatacatccagcaaaggataatagatggccatatttagaaagtagaataacagtatttgaatttcc

SEQ ID NO: 484 - DNA, 120 bp, human papillomavirus
  625339_25353837_60975_HPV18REF.1_1_45
caatttttgtcatttatttcatatactggattgccatttttatcaaatggaaatgcatttggaaattcaaatactgttattctactttctaaatatggccatctattatcctttgctgga

SEQ ID NO: 485 - DNA, 120 bp, human papillomavirus
  625339_25353837_60975_HPV18REF.1_1_46
aaatgcatttccatttgataaaaatggcaatccagtatatgaaataaatgacaaaaattggaaatgtttttttgaaaggacatggtccagattagatttgcacgaggaagaggaagatgc

SEQ ID NO: 486 - DNA, 120 bp, human papillomavirus
  625339_25353837_60975_HPV18REF.1_1_47
ggtctatgattttgtcctgcaacgcacttaaacgttccgaaagggtttccttcggtgtctgcatcttcctcttcctcgtgcaaatctaatctggaccatgtcctttcaaaaaaacatttc

SEQ ID NO: 487 - DNA, 120 bp, human papillomavirus
  625339_25353837_60975_HPV18REF.1_1_48
agacaccgaaggaaaccctttcggaacgtttaagtgcgttgcaggacaaaatcatagaccactatgaaaatgacagtaaagacatagacagccaaatacagtattggcaactaatacgtt

SEQ ID NO: 488 - DNA, 120 bp, human papillomavirus
  625339_25353837_60975_HPV18REF.1_1_49
cctggtggtttaatgtctgtatgccatgttcccttgctgcaaagaatattgcattttcccaacgtattagttgccaatactgtatttggctgtctatgtctttactgtcattttcatagt

SEQ ID NO: 489 - DNA, 120 bp, human papillomavirus
  625339_25353837_60975_HPV18REF.1_1_50
gggaaaatgcaatattctttgcagcaagggaacatggcatacagacattaaaccaccaggtggtgccagcctataacatttcaaaaagtaaagcacataaagctattgaactgcaaatgg

SEQ ID NO: 490 - DNA, 120 bp, human papillomavirus
  625339_25353837_60975_HPV18REF.1_1_51
atgtgtcttgcagtgtccaatcctcggttttgtatgcactttgtgcaaggccttgtagggccatttgcagttcaatagctttatgtgctttactttttgaaatgttataggctggcacca

SEQ ID NO: 491 - DNA, 120 bp, human papillomavirus
  625339_25353837_60975_HPV18REF.1_1_52
ccctacaaggccttgcacaaagtgcatacaaaaccgaggattggacactgcaagacacatgcgaggaactatggaatacagaacctactcactgctttaaaaaaggtggccaaacagtac

SEQ ID NO: 492 - DNA, 120 bp, human papillomavirus
  625339_25353837_60975_HPV18REF.1_1_53
acacactgtcccatgctacataggtcatacaattgtctttgttgccatcaaaatatacttgtactgtttggccaccttttttaaagcagtgagtaggttctgtattccatagttcctcgc

SEQ ID NO: 493 - DNA, 120 bp, human papillomavirus
  625339_25353837_60975_HPV18REF.1_1_54
aagtatattttgatggcaacaaagacaattgtatgacctatgtagcatgggacagtgtgtattatatgactgatgcaggaacatgggacaaaacggctacctgtgtaagtcacaggggat

SEQ ID NO: 494 - DNA, 120 bp, human papillomavirus
  625339_25353837_60975_HPV18REF.1_1_55
tttcacattcacttttaaattctatataaaacgtgttgtacccttcctttacataatacaatcccctgtgacttacacaggtagccgttttgtcccatgttcctgcatcagtcatataat

SEQ ID NO: 495 - DNA, 120 bp, human papillomavirus
  625339_25353837_60975_HPV18REF.1_1_56
tgtattatgtaaaggaagggtacaacacgttttatatagaatttaaaagtgaatgtgaaaaatatgggaacacaggtacgtgggaagtacattttgggaataatgtaattgattgtaatg

SEQ ID NO: 496 - DNA, 120 bp, human papillomavirus
  625339_25353837_60975_HPV18REF.1_1_57
gtagctgtttaacaagctgagtagcggataccgtgtcgtcactggtactgcacatagagtcattacaatcaattacattattcccaaaatgtacttcccacgtacctgtgttcccatatt

SEQ ID NO: 497 - DNA, 120 bp, human papillomavirus
  625339_25353837_60975_HPV18REF.1_1_58
actctatgtgcagtaccagtgacgacacggtatccgctactcagcttgttaaacagctacagcacaccccctcaccgtattccagcaccgtgtccgtgggcaccgcaaagacctacggcc

SEQ ID NO: 498 - DNA, 120 bp, human papillomavirus
  625339_25353837_60975_HPV18REF.1_1_59
gtccacaatgctgcttctccgcgagtccacagtgtccaggtcgtgtagcagccgacgtctggccgtaggtctttgcggtgcccacggacacggtgctggaatacggtgagggggtgtgct

SEQ ID NO: 499 - DNA, 120 bp, human papillomavirus
  625339_25353837_60975_HPV18REF.1_1_60
agacgtcggctgctacacgacctggacactgtggactcgcggagaagcagcattgtggacctgtcaacccacttctcggtgcagctacacctacaggcaacaacaaaagacggaaactct

SEQ ID NO: 500 - DNA, 120 bp, human papillomavirus
  625339_25353837_60975_HPV18REF.1_1_61
aacattttaaactgtttctgtcaccttttaaatgtattataggcgtagtgttaccactacagagtttccgtcttttgttgttgcctgtaggtgtagctgcaccgagaagtgggttgacag

SEQ ID NO: 501 - DNA, 120 bp, human papillomavirus
  625339_25353837_60975_HPV18REF.1_1_62
gtagtggtaacactacgcctataatacatttaaaaggtgacagaaacagtttaaaatgtttacggtacagattgcgaaaacatagcgaccactatagagatatatcatccacctggcatt

SEQ ID NO: 502 - DNA, 120 bp, human papillomavirus
  625339_25353837_60975_HPV18REF.1_1_63
gtgtttcactatggtatgttacagtcagtattcctgttttttcattgcctgcacctgtccaatgccaggtggatgatatatctctatagtggtcgctatgttttcgcaatctgtaccgta

SEQ ID NO: 503 - DNA, 120 bp, human papillomavirus
  625339_25353837_60975_HPV18REF.1_1_64
ggacaggtgcaggcaatgaaaaaacaggaatactgactgtaacataccatagtgaaacacaaagaacaaaatttttaaatactgttgcaattccagatagtgtacaaatattggtgggat

SEQ ID NO: 504 - DNA, 120 bp, human papillomavirus
  625339_25353837_60975_HPV18REF.1_1_65
aaaataaaaaaataagtgataacatattggtactacagcatatgtattacattgtcatgtatcccaccaatatttgtacactatctggaattgcaacagtatttaaaaattttgttcttt

SEQ ID NO: 505 - DNA, 120 bp, human papillomavirus
  625339_25353837_60975_HPV18REF.1_1_66
acatgacaatgtaatacatatgctgtagtaccaatatgttatcacttatttttttattttgcttttgtgtatgcatgtatgtgtgctgccatgtcccgcttttgccatctgtctgtatgt

SEQ ID NO: 506 - DNA, 120 bp, human papillomavirus
  625339_25353837_60975_HPV18REF.1_1_67
ctgtggcaggggacgttattaccacaatatacacaaataccaatacccatgcatacgcacacatacagacagatggcaaaagcgggacatggcagcacacatacatgcatacacaaaagc

SEQ ID NO: 507 - DNA, 120 bp, human papillomavirus
  625339_25353837_60975_HPV18REF.1_1_68
gtgcgtatgcatgggtattggtatttgtgtatattgtggtaataacgtcccctgccacagcattcacagtatatgtattttgttttttattgcccatgttactattgcatatacatgcta

SEQ ID NO: 508 - DNA, 120 bp, human papillomavirus
  625339_25353837_60975_HPV18REF.1_1_69
aaatatacaatgtacaatacactgtataaaacaacctatacaattactgtaaagacaatatagcatgtatatgcaatagtaacatgggcaataaaaaacaaaatacatatactgtgaatg

SEQ ID NO: 509 - DNA, 120 bp, human papillomavirus
  625339_25353837_60975_HPV18REF.1_1_70
tattgtctttacagtaattgtataggttgttttatacagtgtattgtacattgtatattttgttttataccttttatgctttttgtatttttgtaataaaagtatggtatcccaccgtgc

SEQ ID NO: 510 - DNA, 120 bp, human papillomavirus
  625339_25353837_60975_HPV18REF.1_1_71
gtaccagattgtttacatgttttatataagtcagttaccgaagcccgtttgcgtcgtgcggcacggtgggataccatacttttattacaaaaatacaaaaagcataaaaggtataaaaca

SEQ ID NO: 511 - DNA, 120 bp, human papillomavirus
  625339_25353837_60975_HPV18REF.1_1_72
cgcacgacgcaaacgggcttcggtaactgacttatataaaacatgtaaacaatctggtacatgtccacctgatgttgttcctaaggtggagggcaccacgttagcagataaaatattgca

SEQ ID NO: 512 - DNA, 120 bp, human papillomavirus
  625339_25353837_60975_HPV18REF.1_1_73
cctgtaccactgccagtacctatgccaagtccacccaaaaatataccaaggcttgaccattgcaatattttatctgctaacgtggtgccctccaccttaggaacaacatcaggtggacat

SEQ ID NO: 513 - DNA, 120 bp, human papillomavirus
  625339_25353837_60975_HPV18REF.1_1_74
atggtcaagccttggtatatttttgggtggacttggcataggtactggcagtggtacagggggtcgtacagggtacattccattgggtgggcgttccaatacagtggtggatgttggtcc

SEQ ID NO: 514 - DNA, 120 bp, human papillomavirus
  625339_25353837_60975_HPV18REF.1_1_75
aatgtaacaatagatgggtctgtggggcccacaggttcaataaccactgggggacgtgtaggaccaacatccaccactgtattggaacgcccacccaatggaatgtaccctgtacgaccc

SEQ ID NO: 515 - DNA, 120 bp, human papillomavirus
  625339_25353837_60975_HPV18REF.1_1_76
tacacgtcccccagtggttattgaacctgtgggccccacagacccatctattgttacattaatagaggactccagtgtggttacatcaggtgcacctaggcctacgtttactggcacgtc

SEQ ID NO: 516 - DNA, 120 bp, human papillomavirus
  625339_25353837_60975_HPV18REF.1_1_77
gaaggtgtgatatccaaaaccgcaggtgtagttgtacccgcagatgttatatcaaacccagacgtgccagtaaacgtaggcctaggtgcacctgatgtaaccacactggagtcctctatt

SEQ ID NO: 517 - DNA, 120 bp, human papillomavirus
  625339_25353837_60975_HPV18REF.1_1_78
tgggtttgatataacatctgcgggtacaactacacctgcggttttggatatcacaccttcgtctacctctgtgtctatttccacaaccaattttaccaatcctgcattttctgatccgtc

SEQ ID NO: 518 - DNA, 120 bp, human papillomavirus
  625339_25353837_60975_HPV18REF.1_1_79
gtaggggtaccaacaaatacattacctgccacctccccagtttgtggaacttcaataatggacggatcagaaaatgcaggattggtaaaattggttgtggaaatagacacagaggtagac

SEQ ID NO: 519 - DNA, 120 bp, human papillomavirus
  625339_25353837_60975_HPV18REF.1_1_80
cattattgaagttccacaaactggggaggtggcaggtaatgtatttgttggtacccctacatctggaacacatgggtatgaggaaatacctttacaaacatttgcttcttctggtacggg

SEQ ID NO: 520 - DNA, 120 bp, human papillomavirus
  625339_25353837_60975_HPV18REF.1_1_81
aggcggggacctgctacacgccgcacagtaggcaatggggtactactaatgggttcctcccccgtaccagaagaagcaaatgtttgtaaaggtatttcctcatacccatgtgttccagat

SEQ ID NO: 521 - DNA, 120 bp, human papillomavirus
  625339_25353837_60975_HPV18REF.1_1_82
ggaggaacccattagtagtaccccattgcctactgtgcggcgtgtagcaggtccccgcctttacagtagggcctaccaacaagtgtcagtggctaaccctgagtttcttacacgtccatc

SEQ ID NO: 522 - DNA, 120 bp, human papillomavirus
  625339_25353837_60975_HPV18REF.1_1_83
tcaaatgttaatgtagtgtccacaggctcaaaggccgggttgtcatatgtaattaaagaggatggacgtgtaagaaactcagggttagccactgacacttgttggtaggccctactgtaa

SEQ ID NO: 523 - DNA, 120 bp, human papillomavirus
  625339_25353837_60975_HPV18REF.1_1_84
ctctttaattacatatgacaacccggcctttgagcctgtggacactacattaacatttgatcctcgtagtgatgttcctgattcagattttatggatattatccgtctacataggcctgc

SEQ ID NO: 524 - DNA, 120 bp, human papillomavirus
  625339_25353837_60975_HPV18REF.1_1_85
aacatagttgcccgttgacctaatctactaaagcgaacagtcccacgcctggatgttaaagcaggcctatgtagacggataatatccataaaatctgaatcaggaacatcactacgagga

SEQ ID NO: 525 - DNA, 120 bp, human papillomavirus
  625339_25353837_60975_HPV18REF.1_1_86
tttaacatccaggcgtgggactgttcgctttagtagattaggtcaacgggcaactatgtttacccgcagcggtacacaaataggtgctagggttcacttttatcatgatataagtcctat

SEQ ID NO: 526 - DNA, 120 bp, human papillomavirus
  625339_25353837_60975_HPV18REF.1_1_87
tcattgtcctccgtggcagatactaaaggctgcagttcaatatattctggggaaggtgcaataggacttatatcatgataaaagtgaaccctagcacctatttgtgtaccgctgcgggta

SEQ ID NO: 527 - DNA, 120 bp, human papillomavirus
  625339_25353837_60975_HPV18REF.1_1_88
tgcaccttccccagaatatattgaactgcagcctttagtatctgccacggaggacaatgacttgtttgatatatatgcagatgacatggaccctgcagtgcctgtaccatcgcgttctac

SEQ ID NO: 528 - DNA, 120 bp, human papillomavirus
  625339_25353837_60975_HPV18REF.1_1_89
ttactataggaagaggcagaagatatagtgggcgaatatttaaaaaatgcaaaggaggtagtagaacgcgatggtacaggcactgcagggtccatgtcatctgcatatatatcaaacaag

SEQ ID NO: 529 - DNA, 120 bp, human papillomavirus
  625339_25353837_60975_HPV18REF.1_1_90
tacctcctttgcattttttaaatattcgcccactatatcttctgcctcttcctatagtaatgtaacggtccctttaacctcctcttgggatgtgcctgtatacacgggtcctgatattac

SEQ ID NO: 530 - DNA, 120 bp, human papillomavirus
  625339_25353837_60975_HPV18REF.1_1_91
tgtgtagaggcaggggccgtgggtgatacaatgggccatacagaggtagtagatggtaatgtaatatcaggacccgtgtatacaggcacatcccaagaggaggttaaagggaccgttaca

SEQ ID NO: 531 - DNA, 120 bp, human papillomavirus
  625339_25353837_60975_HPV18REF.1_1_92
attaccatctactacctctgtatggcccattgtatcacccacggcccctgcctctacacagtatattggtatacatggtacacattattatttgtggccattatattattttattcctaa

SEQ ID NO: 532 - DNA, 120 bp, human papillomavirus
  625339_25353837_60975_HPV18REF.1_1_93
tattgtcactaggccgccacaaagccatctgcaaaaaaatagggaacacgtttacgtttcttaggaataaaataatataatggccacaaataataatgtgtaccatgtataccaatatac

SEQ ID NO: 533 - DNA, 120 bp, human papillomavirus
  625339_25353837_60975_HPV18REF.1_1_94
gaaacgtaaacgtgttccctatttttttgcagatggctttgtggcggcctagtgacaataccgtatatcttccacctccttctgtggcaagagttgtaaataccgatgattatgtgactc

SEQ ID NO: 534 - DNA, 120 bp, human papillomavirus
  625339_25353837_60975_HPV18REF.1_1_95
aatatggattaccaacagttaataatctagagctgccagcatgataaaatatgcttgtgcgagtcacataatcatcggtatttacaactcttgccacagaaggaggtggaagatatacgg

SEQ ID NO: 535 - DNA, 120 bp, human papillomavirus
  625339_25353837_60975_HPV18REF.1_1_96
gcacaagcatattttatcatgctggcagctctagattattaactgttggtaatccatattttagggttcctgcaggtggtggcaataagcaggatattcctaaggtttctgcataccaat

SEQ ID NO: 536 - DNA, 120 bp, human papillomavirus
  625339_25353837_60975_HPV18REF.1_1_97
tactagtatcaggtaaaccaaatttatttgggtcaggtaactgcaccctaaatactctatattggtatgcagaaaccttaggaatatcctgcttattgccaccacctgcaggaaccctaa

SEQ ID NO: 537 - DNA, 120 bp, human papillomavirus
  625339_25353837_60975_HPV18REF.1_1_98
atagagtatttagggtgcagttacctgacccaaataaatttggtttacctgatactagtatttataatcctgaaacacaacgtttagtgtgggcctgtgctggagtggaaattggccgtg

SEQ ID NO: 538 - DNA, 120 bp, human papillomavirus
  625339_25353837_60975_HPV18REF.1_1_99
cagtgtcatctaatttattataaaatggatgcccactaaggccaacacctaaaggctgaccacggccaatttccactccagcacaggcccacactaaacgttgtgtttcaggattataaa

SEQ ID NO: 539 - DNA, 120 bp, human papillomavirus
  625339_25353837_60975_HPV18REF.1_1_100
gtcagcctttaggtgttggccttagtgggcatccattttataataaattagatgacactgaaagttcccatgccgccacgtctaatgtttctgaggacgttagggacaatgtgtctgtag

SEQ ID NO: 540 - DNA, 120 bp, human papillomavirus
  625339_25353837_60975_HPV18REF.1_1_101
cccagtgttccccaatagcaggggcacagcccaaaatacataactgtgtctgcttataatctacagacacattgtccctaacgtcctcagaaacattagacgtggcggcatgggaacttt

SEQ ID NO: 541 - DNA, 120 bp, human papillomavirus
  625339_25353837_60975_HPV18REF.1_1_102
attataagcagacacagttatgtattttgggctgtgcccctgctattggggaacactgggctaaaggcactgcttgtaaatcgcgtcctttatcacagggcgattgcccccctttagaac

SEQ ID NO: 542 - DNA, 120 bp, human papillomavirus
  625339_25353837_60975_HPV18REF.1_1_103
agtccatggcaccatatccagtatctaccatatcaccatcttccaaaactgtgtttttaagttctaaaggggggcaatcgccctgtgataaaggacgcgatttacaagcagtgcctttag

SEQ ID NO: 543 - DNA, 120 bp, human papillomavirus
  625339_25353837_60975_HPV18REF.1_1_104
ttaaaaacacagttttggaagatggtgatatggtagatactggatatggtgccatggactttagtacattgcaagatactaaatgtgaggtaccattggatatttgtcagtctatttgta

SEQ ID NO: 544 - DNA, 120 bp, human papillomavirus
  625339_25353837_60975_HPV18REF.1_1_105
agcaaaaaaacatggaatccccataaggatctgcagacatttgtaaataatcaggatatttacaaatagactgacaaatatccaatggtacctcacatttagtatcttgcaatgtactaa

SEQ ID NO: 545 - DNA, 120 bp, human papillomavirus
  625339_25353837_60975_HPV18REF.1_1_106
aatatcctgattatttacaaatgtctgcagatccttatggggattccatgtttttttgcttacggcgtgagcagctttttgctaggcatttttggaatagagcaggtactatgggtgaca

SEQ ID NO: 546 - DNA, 120 bp, human papillomavirus
  625339_25353837_60975_HPV18REF.1_1_107
cacagctgccaggtgaagcacgcatacctgtgcctttaatatataaggattgaggcacagtgtcacccatagtacctgctctattccaaaaatgcctagcaaaaagctgctcacgccgta

SEQ ID NO: 547 - DNA, 120 bp, human papillomavirus
  625339_25353837_60975_HPV18REF.1_1_108
ctgtgcctcaatccttatatattaaaggcacaggtatgcgtgcttcacctggcagctgtgtgtattctccctctccaagtggctctattgttacctctgactcccagttgtttaataaac

SEQ ID NO: 548 - DNA, 120 bp, human papillomavirus
  625339_25353837_60975_HPV18REF.1_1_109
ataattgattatgccagcaaacaccattgttatgaccctgtgccttatgtaaccaatatggtttattaaacaactgggagtcagaggtaacaatagagccacttggagagggagaataca

SEQ ID NO: 549 - DNA, 120 bp, human papillomavirus
  625339_25353837_60975_HPV18REF.1_1_110
catattggttacataaggcacagggtcataacaatggtgtttgctggcataatcaattatttgttactgtggtagataccactcgcagtaccaatttaacaatatgtgcttctacacagt

SEQ ID NO: 550 - DNA, 120 bp, human papillomavirus
  625339_25353837_60975_HPV18REF.1_1_111
cctcaacatgtctgctatactgcttaaatttggtagcatcatattgcccaggtacaggagactgtgtagaagcacatattgttaaattggtactgcgagtggtatctaccacagtaacaa

SEQ ID NO: 551 - DNA, 120 bp, human papillomavirus
  625339_25353837_60975_HPV18REF.1_1_112
ctcctgtacctgggcaatatgatgctaccaaatttaagcagtatagcagacatgttgaggaatatgatttgcagtttatttttcagttgtgtactattactttaactgcagatgttatgt

SEQ ID NO: 552 - DNA, 120 bp, human papillomavirus
  625339_25353837_60975_HPV18REF.1_1_113
gggggggaacaccaaagttccaatcctctaaaatactgctattcatactatgaatataggacataacatctgcagttaaagtaatagtacacaactgaaaaataaactgcaaatcatatt

SEQ ID NO: 553 - DNA, 120 bp, human papillomavirus
  625339_25353837_60975_HPV18REF.1_1_114
cctatattcatagtatgaatagcagtattttagaggattggaactttggtgttccccccccgccaactactagtttggtggatacatatcgttttgtacaatctgttgctattacctgtc

SEQ ID NO: 554 - DNA, 120 bp, human papillomavirus
  625339_25353837_60975_HPV18REF.1_1_115
cattccaaaactttaacttatcatagggatccttattttcagccggtgcagcatccttttgacaggtaatagcaacagattgtacaaaacgatatgtatccaccaaactagtagttggcg

SEQ ID NO: 555 - DNA, 120 bp, human papillomavirus
  625339_25353837_60975_HPV18REF.1_1_116
aaaaggatgctgcaccggctgaaaataaggatccctatgataagttaaagttttggaatgtggatttaaaggaaaagttttctttagacttagatcaatatccccttggacgtaaatttt

SEQ ID NO: 556 - DNA, 120 bp, human papillomavirus
  625339_25353837_60975_HPV18REF.1_1_117
atggagcagaacgtttgcgagggcctatggtgggcttgcgacgcaatccagcctgaaccaaaaatttacgtccaaggggatattgatctaagtctaaagaaaacttttcctttaaatcca

SEQ ID NO: 557 - DNA, 120 bp, human papillomavirus
  625339_25353837_60975_HPV18REF.1_1_118
tggttcaggctggattgcgtcgcaagcccaccataggccctcgcaaacgttctgctccatctgccactacgtcttctaaacctgccaagcgtgtgcgtgtacgtgccaggaagtaatatg

SEQ ID NO: 558 - DNA, 120 bp, human papillomavirus
  625339_25353837_60975_HPV18REF.1_1_119
acaaacacaaacacaggacatacaaacacaacaatagatgtatatatatatacacacacacatattacttcctggcacgtacacgcacacgcttggcaggtttagaagacgtagtggcag

SEQ ID NO: 559 - DNA, 120 bp, human papillomavirus
  625339_25353837_60975_HPV18REF.1_1_120
tgtgtgtgtatatatatatacatctattgttgtgtttgtatgtcctgtgtttgtgtttgttgtatgattgcattgtatggtatgtatggttgttgttgtatgttgtatgttactatattt

SEQ ID NO: 560 - DNA, 120 bp, human papillomavirus
  625339_25353837_60975_HPV18REF.1_1_121
ggcgcaaccacataacacacagaaccacaaaacatattttatttaatgccacataccaacaaatatagtaacatacaacatacaacaacaaccatacataccatacaatgcaatcataca

SEQ ID NO: 561 - DNA, 120 bp, human papillomavirus
  625339_25353837_60975_HPV18REF.1_1_122
gttggtatgtggcattaaataaaatatgttttgtggttctgtgtgttatgtggttgcgccctagtgagtaacaactgtatttgtgtttgtggtatgggtgttgcttgttgggctatatat

SEQ ID NO: 562 - DNA, 120 bp, human papillomavirus
  625339_25353837_60975_HPV18REF.1_1_123
gattgtaggataaaatggatgctgtaaggtgtgcagttttataacttgaaatacaggacaatatatagcccaacaagcaacacccataccacaaacacaaatacagttgttactcactag

SEQ ID NO: 563 - DNA, 120 bp, human papillomavirus
  625339_25353837_60975_HPV18REF.1_1_124
tgtcctgtatttcaagttataaaactgcacaccttacagcatccattttatcctacaatcctccattttgctgtgcaaccgatttcggttgcctttggcttatgtctgtggttttctgca

SEQ ID NO: 564 - DNA, 120 bp, human papillomavirus
  625339_25353837_60975_HPV18REF.1_1_125
aatatgtaggagcagtgcccaaaagattaaagtttgcaatagtgccagcgtactgtattgtgcagaaaaccacagacataagccaaaggcaaccgaaatcggttgcacagcaaaatggag

SEQ ID NO: 565 - DNA, 120 bp, human papillomavirus
  625339_25353837_60975_HPV18REF.1_1_126
caatacagtacgctggcactattgcaaactttaatcttttgggcactgctcctacatattttgaacaattggcgcgcctctttggcgcatataaggcgcacctggtattagtcattttcc

SEQ ID NO: 566 - DNA, 120 bp, human papillomavirus
  625339_25353837_60975_HPV18REF.1_1_127
gttttattacttagggagtggatatagttatgcaagcaattgttgtagcgcacctggacaggaaaatgactaataccaggtgcgccttatatgcgccaaagaggcgcgccaattgttcaa

SEQ ID NO: 567 - DNA, 120 bp, human papillomavirus
  625339_25353837_60975_HPV18REF.1_1_128
tgtccaggtgcgctacaacaattgcttgcataactatatccactccctaagtaataaaactgcttttaggcacatattttagtttgtttttacttaagctaattgcatacttggcttgta

SEQ ID NO: 568 - DNA, 120 bp, human papillomavirus
  625339_25353837_60975_HPV18REF.1_1_129
gcttagtcatattatagttcatgttaagggtagacagaatgttggacatgaaagtagttgtacaagccaagtatgcaattagcttaagtaaaaacaaactaaaatatgtgcctaaaagca

SEQ ID NO: 569 - DNA, 117 bp, human papillomavirus
  625339_25353837_60975_HPV18REF.1_1_130
caactactttcatgtccaacattctgtctacccttaacatgaactataatatgactaagctgtgcatacatagtttatgcaaccgaaataggttgggcagcacatactatacttttc

SEQ ID NO: 570 - DNA, 120 bp, human papillomavirus
  625339_25353838_333048_HPV31REF.1_1_1
gatttttgaacatggcgtctgtaggtttgcacaaaatactatgtgctttatataccaaccgttttcggttcaccactttcggtcactccctacttttttatactaagattattattatta

SEQ ID NO: 571 - DNA, 120 bp, human papillomavirus
  625339_25353838_333048_HPV31REF.1_1_2
ggttggtatataaagcacatagtattttgtgcaaacctacagacgccatgttcaaaaatcctgcagaaagacctcggaaattgcatgaactaagctcggcattggaaataccctacgatg

SEQ ID NO: 572 - DNA, 120 bp, human papillomavirus
  625339_25353838_333048_HPV31REF.1_1_3
aatctaatacctctgtttctgttaactgacctttgcagtagacacaattcaatcttagttcatcgtagggtatttccaatgccgagcttagttcatgcaatttccgaggtctttctgcag

SEQ ID NO: 573 - DNA, 120 bp, human papillomavirus
  625339_25353838_333048_HPV31REF.1_1_4
aactaagattgaattgtgtctactgcaaaggtcagttaacagaaacagaggtattagattttgcatttacagatttaacaatagtatatagggacgacacaccacacggagtgtgtacaa

SEQ ID NO: 574 - DNA, 120 bp, human papillomavirus
  625339_25353838_333048_HPV31REF.1_1_5
catacacactatatctataccatctaaattcacttacttttgaataaaatcttaaacattttgtacacactccgtgtggtgtgtcgtccctatatactattgttaaatctgtaaatgcaa

SEQ ID NO: 575 - DNA, 120 bp, human papillomavirus
  625339_25353838_333048_HPV31REF.1_1_6
aatgtttaagattttattcaaaagtaagtgaatttagatggtatagatatagtgtgtatggaacaacattagaaaaattgacaaacaaaggtatatgtgatttgttaattaggtgtataa

SEQ ID NO: 576 - DNA, 120 bp, human papillomavirus
  625339_25353838_333048_HPV31REF.1_1_7
atcgtttctttttatccaaatgtctttgtttttcttctggacacaacggtctttgacacgttatacacctaattaacaaatcacatatacctttgtttgtcaatttttctaatgttgttc

SEQ ID NO: 577 - DNA, 120 bp, human papillomavirus
  625339_25353838_333048_HPV31REF.1_1_8
cgtgtcaaagaccgttgtgtccagaagaaaaacaaagacatttggataaaaagaaacgattccacaacataggaggaaggtggacaggacgttgcatagcatgttggagaagacctcgta

SEQ ID NO: 578 - DNA, 120 bp, human papillomavirus
  625339_25353838_333048_HPV31REF.1_1_9
tctaacacatagtcttgcaacgtaggtgtttctccacgcatgtttacacttgggtttcagtacgaggtcttctccaacatgctatgcaacgtcctgtccaccttcctcctatgttgtgga

SEQ ID NO: 579 - DNA, 120 bp, human papillomavirus
  625339_25353838_333048_HPV31REF.1_1_10
ctgaaacccaagtgtaaacatgcgtggagaaacacctacgttgcaagactatgtgttagatttgcaacctgaggcaactgacctccactgttatgagcaattacccgacagctcagatga

SEQ ID NO: 580 - DNA, 120 bp, human papillomavirus
  625339_25353838_333048_HPV31REF.1_1_11
atattgtaattggatgtgtccggttctgcttgtccagctggactgtctatgacatcctcctcatctgagctgtcgggtaattgctcataacagtggaggtcagttgcctcaggttgcaaa

SEQ ID NO: 581 - DNA, 120 bp, human papillomavirus
  625339_25353838_333048_HPV31REF.1_1_12
ggaggatgtcatagacagtccagctggacaagcagaaccggacacatccaattacaatatcgttaccttttgttgtcagtgtaagtctacacttcgtttgtgtgtacagagcacacaagt

SEQ ID NO: 582 - DNA, 120 bp, human papillomavirus
  625339_25353838_333048_HPV31REF.1_1_13
cagttggggcacacgattccaaatgagcccattaacagctcttgcaatatgcgaatatctacttgtgtgctctgtacacacaaacgaagtgtagacttacactgacaacaaaaggtaacg

SEQ ID NO: 583 - DNA, 120 bp, human papillomavirus
  625339_25353838_333048_HPV31REF.1_1_14
agatattcgcatattgcaagagctgttaatgggctcatttggaatcgtgtgccccaactgttctactagactgtaactacaatggctgatccagcaggtacagatggggaggggacggga

SEQ ID NO: 584 - DNA, 120 bp, human papillomavirus
  625339_25353838_333048_HPV31REF.1_1_15
tgaaatgttgtcccctgtctgtctgtcaattactgcttctacataaaaccaaccattgcatcccgtcccctccccatctgtacctgctggatcagccattgtagttacagtctagtagaa

SEQ ID NO: 585 - DNA, 120 bp, human papillomavirus
  625339_25353838_333048_HPV31REF.1_1_16
tgcaatggttggttttatgtagaagcagtaattgacagacagacaggggacaacatttcagaggacgaaaatgaagacagtagtgatactggggaggatatggttgactttattgacaat

SEQ ID NO: 586 - DNA, 120 bp, human papillomavirus
  625339_25353838_333048_HPV31REF.1_1_17
ctgtgcatgaaacaatgcctgtgctgtctctgcttctgcctgattgttgtatacattacaattgtcaataaagtcaaccatatcctccccagtatcactactgtcttcattttcgtcctc

SEQ ID NO: 587 - DNA, 120 bp, human papillomavirus
  625339_25353838_333048_HPV31REF.1_1_18
tgtaatgtatacaacaatcaggcagaagcagagacagcacaggcattgtttcatgcacaggaagcggaggaacatgcagaggctgtgcaggttctaaaacgaaagtatgtaggtagtcct

SEQ ID NO: 588 - DNA, 120 bp, human papillomavirus
  625339_25353838_333048_HPV31REF.1_1_19
gcatatagcttttaaccgtggactaatattataatccacacaactactaatatcacttaaaggactacctacatactttcgttttagaacctgcacagcctctgcatgttcctccgcttc

SEQ ID NO: 589 - DNA, 120 bp, human papillomavirus
  625339_25353838_333048_HPV31REF.1_1_20
ttaagtgatattagtagttgtgtggattataatattagtccacggttaaaagctatatgcatagaaaataacagtaaaacagcaaaacgaagactctttgaacttccagacagcgggtat

SEQ ID NO: 590 - DNA, 120 bp, human papillomavirus
  625339_25353838_333048_HPV31REF.1_1_21
taatgttgtttgttgctcctctacctgtaccatctgctgcgtttccacttcagtattgccatacccgctgtctggaagttcaaagagtcttcgttttgctgttttactgttattttctat

SEQ ID NO: 591 - DNA, 120 bp, human papillomavirus
  625339_25353838_333048_HPV31REF.1_1_22
ggcaatactgaagtggaaacgcagcagatggtacaggtagaggagcaacaaacaacattaagttgtaatggtagtgacgggacacatagtgaacgagagaatgaaactccaacacgtaat

SEQ ID NO: 592 - DNA, 120 bp, human papillomavirus
  625339_25353838_333048_HPV31REF.1_1_23
ttctttaaatttacctaacatagcagctttaccattgctagtttttaacacttgcaatatattacgtgttggagtttcattctctcgttcactatgtgtcccgtcactaccattacaact

SEQ ID NO: 593 - DNA, 120 bp, human papillomavirus
  625339_25353838_333048_HPV31REF.1_1_24
atattgcaagtgttaaaaactagcaatggtaaagctgctatgttaggtaaatttaaagaattatatggtgtaagttttatggaactaattaggccatttcaaagcaataaaagcacatgt

SEQ ID NO: 594 - DNA, 120 bp, human papillomavirus
  625339_25353838_333048_HPV31REF.1_1_25
ggttttaaatccttctgcaactgtacctgtaactccaaacgcagctacacaccaatcagtacatgtgcttttattgctttgaaatggcctaattagttccataaaacttacaccatataa

SEQ ID NO: 595 - DNA, 120 bp, human papillomavirus
  625339_25353838_333048_HPV31REF.1_1_26
actgattggtgtgtagctgcgtttggagttacaggtacagttgcagaaggatttaaaaccctattgcaaccatattgtttgtattgccatttacaaagtttagcatgttcctggggcatg

SEQ ID NO: 596 - DNA, 120 bp, human papillomavirus
  625339_25353838_333048_HPV31REF.1_1_27
taatttttcaattgttattctattttttgcacatttaaatctcacaagcattaacataaccatgccccaggaacatgctaaactttgtaaatggcaatacaaacaatatggttgcaatag

SEQ ID NO: 597 - DNA, 120 bp, human papillomavirus
  625339_25353838_333048_HPV31REF.1_1_28
gttatgttaatgcttgtgagatttaaatgtgcaaaaaatagaataacaattgaaaaattattagaaaaattattgtgtatatctacaaattgtatgttaattcagccacccaaattacgt

SEQ ID NO: 598 - DNA, 120 bp, human papillomavirus
  625339_25353838_333048_HPV31REF.1_1_29
atatacatcgctaatgtttgacattcctgttctgtaccaatataatgctgcagctgtgctacgtaatttgggtggctgaattaacatacaatttgtagatatacacaataatttttctaa

SEQ ID NO: 599 - DNA, 120 bp, human papillomavirus
  625339_25353838_333048_HPV31REF.1_1_30
agcacagctgcagcattatattggtacagaacaggaatgtcaaacattagcgatgtatatggtgaaacaccagaatggatagaaagacaaacagtattacagcatagttttaatgacaca

SEQ ID NO: 600 - DNA, 120 bp, human papillomavirus
  625339_25353838_333048_HPV31REF.1_1_31
actatcatccataacatcattgtcatatgcccattgtaccatttgggacaaatcaaatgttgtgtcattaaaactatgctgtaatactgtttgtctttctatccattctggtgtttcacc

SEQ ID NO: 601 - DNA, 120 bp, human papillomavirus
  625339_25353838_333048_HPV31REF.1_1_32
acatttgatttgtcccaaatggtacaatgggcatatgacaatgatgttatggatgatagtgaaattgcctataaatatgcacaattagctgacagtgatagtaatgcatgtgcattttta

SEQ ID NO: 602 - DNA, 120 bp, human papillomavirus
  625339_25353838_333048_HPV31REF.1_1_33
tttataatgtctacacattgttccacaatctttaactatttttgcctgcgaattactttttaaaaatgcacatgcattactatcactgtcagctaattgtgcatatttataggcaatttc

SEQ ID NO: 603 - DNA, 120 bp, human papillomavirus
  625339_25353838_333048_HPV31REF.1_1_34
aaaagtaattcgcaggcaaaaatagttaaagattgtggaacaatgtgtagacattataaacgagcagaaaaacgacaaatgtccatgggacagtggattaaaagtagatgtgacaaagtt

SEQ ID NO: 604 - DNA, 120 bp, human papillomavirus
  625339_25353838_333048_HPV31REF.1_1_35
aaattctatttgttgatatcttaaaaactttactatgtccctccagtcaccttcgtcactaactttgtcacatctacttttaatccactgtcccatggacatttgtcgtttttctgctcg

SEQ ID NO: 605 - DNA, 120 bp, human papillomavirus
  625339_25353838_333048_HPV31REF.1_1_36
agtgacgaaggtgactggagggacatagtaaagtttttaagatatcaacaaatagaatttgtgtcatttttatctgcattaaagctgtttttaaaaggagtgccaaagaaaaactgtatt

SEQ ID NO: 606 - DNA, 120 bp, human papillomavirus
  625339_25353838_333048_HPV31REF.1_1_37
aaagctaataaggctcattccaaaatatgatttacctgtattaggtgcaccatgtattaaaatacagtttttctttggcactccttttaaaaacagctttaatgcagataaaaatgacac

SEQ ID NO: 607 - DNA, 120 bp, human papillomavirus
  625339_25353838_333048_HPV31REF.1_1_38
ttaatacatggtgcacctaatacaggtaaatcatattttggaatgagccttattagctttttacaaggatgtataatatcatatgcaaattcaaaaagtcatttttggttacaaccactg

SEQ ID NO: 608 - DNA, 120 bp, human papillomavirus
  625339_25353838_333048_HPV31REF.1_1_39
gtctatataatgccaacatggcgttgtagcatcatctaacatgcctattttagcatcagccagtggttgtaaccaaaaatgactttttgaatttgcatatgatattatacatccttgtaa

SEQ ID NO: 609 - DNA, 120 bp, human papillomavirus
  625339_25353838_333048_HPV31REF.1_1_40
gctgatgctaaaataggcatgttagatgatgctacaacgccatgttggcattatatagacaattacctacgaaatgcactagatggcaaccctgtatctatagatgtaaagcataaagct

SEQ ID NO: 610 - DNA, 120 bp, human papillomavirus
  625339_25353838_333048_HPV31REF.1_1_41
atccttacctgcatttatattagatgtaatcaataaaggaggacattttaactgcattaaagctttatgctttacatctatagatacagggttgccatctagtgcatttcgtaggtaatt

SEQ ID NO: 611 - DNA, 120 bp, human papillomavirus
  625339_25353838_333048_HPV31REF.1_1_42
ttaatgcagttaaaatgtcctcctttattgattacatctaatataaatgcaggtaaggatgacagatggccatacctacatagcagactggtggtttttacatttccaaatccatttcca

SEQ ID NO: 612 - DNA, 120 bp, human papillomavirus
  625339_25353838_333048_HPV31REF.1_1_43
gaaaaaggatttccagtttttatcacttaattcatatactggatttccgtttttgtcaaatggaaatggatttggaaatgtaaaaaccaccagtctgctatgtaggtatggccatctgtc

SEQ ID NO: 613 - DNA, 120 bp, human papillomavirus
  625339_25353838_333048_HPV31REF.1_1_44
tttgacaaaaacggaaatccagtatatgaattaagtgataaaaactggaaatcctttttctcaaggacgtggtgcagattaaatttgcacgaggaagaggacaaagaaaacgatggagac

SEQ ID NO: 614 - DNA, 120 bp, human papillomavirus
  625339_25353838_333048_HPV31REF.1_1_45
ctatcattttcataatgttctaatattttgtcctgacacacatttaaacgttgagaaagagtctccatcgttttctttgtcctcttcctcgtgcaaatttaatctgcaccacgtccttga

SEQ ID NO: 615 - DNA, 120 bp, human papillomavirus
  625339_25353838_333048_HPV31REF.1_1_46
tctttctcaacgtttaaatgtgtgtcaggacaaaatattagaacattatgaaaatgatagtaaacgactttgtgatcatatagactattggaaacatattcgacttgaatgtgtattaat

SEQ ID NO: 616 - DNA, 120 bp, human papillomavirus
  625339_25353838_333048_HPV31REF.1_1_47
gacaacgctggcaccacctggtggttaatactgtgtattcccatttctcttgctttatacattaatacacattcaagtcgaatatgtttccaatagtctatatgatcacaaagtcgttta

SEQ ID NO: 617 - DNA, 120 bp, human papillomavirus
  625339_25353838_333048_HPV31REF.1_1_48
gtataaagcaagagaaatgggaatacacagtattaaccaccaggtggtgccagcgttgtcagtatcaaaggccaaagccttacaagctattgaactacaaatgatgttggaaacattaaa

SEQ ID NO: 618 - DNA, 120 bp, human papillomavirus
  625339_25353838_333048_HPV31REF.1_1_49
aaatacagttcaagacttgtttgctgcattgtccagtcctcatttttgtattcagtgttatttaatgtttccaacatcatttgtagttcaatagcttgtaaggctttggcctttgatact

SEQ ID NO: 619 - DNA, 120 bp, human papillomavirus
  625339_25353838_333048_HPV31REF.1_1_50
taacactgaatacaaaaatgaggactggacaatgcagcaaacaagtcttgaactgtatttaactgcacctacagggtgtttaaaaaaacatggatatactgtagaggtgcaatttgatgg

SEQ ID NO: 620 - DNA, 120 bp, human papillomavirus
  625339_25353838_333048_HPV31REF.1_1_51
ccatctatacataggtatataaatttccagttagtataatgcatggtgttgtgtacatcaccatcaaattgcacctctacagtatatccatgtttttttaaacaccctgtaggtgcagtt

SEQ ID NO: 621 - DNA, 120 bp, human papillomavirus
  625339_25353838_333048_HPV31REF.1_1_52
tgatgtacacaacaccatgcattatactaactggaaatttatatacctatgtatagatggccaatgtactgttgtggaagggcaagttaattgtaagggcatttattatgtacatgaagg

SEQ ID NO: 622 - DNA, 120 bp, human papillomavirus
  625339_25353838_333048_HPV31REF.1_1_53
tttttaccagtcccatatttttttgcctcttctgtaaaatttacaaaatatgttatatgtccttcatgtacataataaatgcccttacaattaacttgcccttccacaacagtacattgg

SEQ ID NO: 623 - DNA, 120 bp, human papillomavirus
  625339_25353838_333048_HPV31REF.1_1_54
acatataacatattttgtaaattttacagaagaggcaaaaaaatatgggactggtaaaaaatgggaagtgcatgcgggtggtcaggtaattgtttttcctgaatctgtatttagcagtga

SEQ ID NO: 624 - DNA, 120 bp, human papillomavirus
  625339_25353838_333048_HPV31REF.1_1_55
gatgtggtggtgttgttggctgttggtagctttgtaacaatcccagcaaaggatatttcgtcactgctaaatacagattcaggaaaaacaattacctgaccacccgcatgcacttcccat

SEQ ID NO: 625 - DNA, 120 bp, human papillomavirus
  625339_25353838_333048_HPV31REF.1_1_56
cgaaatatcctttgctgggattgttacaaagctaccaacagccaacaacaccaccacatcgaattccaaaacctgcgccttgggcaccagtgaaggtgtgcggcgggcgacgacgtctac

SEQ ID NO: 626 - DNA, 120 bp, human papillomavirus
  625339_25353838_333048_HPV31REF.1_1_57
cgcaacaacttgttggggtggtgggtgtttctgtgctctggctctgttcttggtcgcttagtagacgtcgtcgcccgccgcacaccttcactggtgcccaaggcgcaggttttggaattc

SEQ ID NO: 627 - DNA, 120 bp, human papillomavirus
  625339_25353838_333048_HPV31REF.1_1_58
taagcgaccaagaacagagccagagcacagaaacacccaccaccccaacaagttgttgcgaggcgactccgtggacagtgtcaactgtggggttatcagtgcagctgcatgcacaaacca

SEQ ID NO: 628 - DNA, 120 bp, human papillomavirus
  625339_25353838_333048_HPV31REF.1_1_59
tttgcatcaccttttaagtgtattataggtgtagttgcaggacaactgacagcccttgtttggtttgtgcatgcagctgcactgataaccccacagttgacactgtccacggagtcgcct

SEQ ID NO: 629 - DNA, 120 bp, human papillomavirus
  625339_25353838_333048_HPV31REF.1_1_60
aacaagggctgtcagttgtcctgcaactacacctataatacacttaaaaggtgatgcaaatatattaaaatgtttaagatataggctgtcaaaatataaacaattgtatgaacaagtgtc

SEQ ID NO: 630 - DNA, 120 bp, human papillomavirus
  625339_25353838_333048_HPV31REF.1_1_61
gttaaggttacaatagcatttttatgttttccatctgtacatgtccaatgccatgtagatgacacttgttcatacaattgtttatattttgacagcctatatcttaaacattttaatata

SEQ ID NO: 631 - DNA, 120 bp, human papillomavirus
  625339_25353838_333048_HPV31REF.1_1_62
atctacatggcattggacatgtacagatggaaaacataaaaatgctattgtaaccttaacatatataagtacatcacaaagagacgattttttaaatactgtaaaaatacctaacacagt

SEQ ID NO: 632 - DNA, 120 bp, human papillomavirus
  625339_25353838_333048_HPV31REF.1_1_63
ctgtagaaatatttagttcaatcattaggctaaatagtcatatatcctgttgacactgatactgtgttaggtatttttacagtatttaaaaaatcgtctctttgtgatgtacttatatat

SEQ ID NO: 633 - DNA, 120 bp, human papillomavirus
  625339_25353838_333048_HPV31REF.1_1_64
atcagtgtcaacaggatatatgactatttagcctaatgattgaactaaatatttctacagtaagcattgtgctatgctttttgctttgcttttgtgtgctactatttgtgtgtcttgtca

SEQ ID NO: 634 - DNA, 120 bp, human papillomavirus
  625339_25353838_333048_HPV31REF.1_1_65
aaatcacaattaataatagtagtgttgcatataccgacacagacagcacaagtggacgtatgacaagacacacaaatagtagcacacaaaagcaaagcaaaaagcatagcacaatgctta

SEQ ID NO: 635 - DNA, 120 bp, human papillomavirus
  625339_25353838_333048_HPV31REF.1_1_66
tacgtccacttgtgctgtctgtgtcggtatatgcaacactactattattaattgtgattttatgggttattgcaacctctccattacgttgtttttgtatatatgttgtgtttatatata

SEQ ID NO: 636 - DNA, 120 bp, human papillomavirus
  625339_25353838_333048_HPV31REF.1_1_67
agtaaaaaagttactgttgacttaaaaaagatgcatgtgtatgaattacaaataatggaatatatataaacacaacatatatacaaaaacaacgtaatggagaggttgcaataacccata

SEQ ID NO: 637 - DNA, 120 bp, human papillomavirus
  625339_25353838_333048_HPV31REF.1_1_68
ttccattatttgtaattcatacacatgcatcttttttaagtcaacagtaacttttttacttgtgtatactgttgtttgtattggtattggtattggtattggtattggtattggtataat

SEQ ID NO: 638 - DNA, 120 bp, human papillomavirus
  625339_25353838_333048_HPV31REF.1_1_69
agtgcgttttgtagagcgtttggaccgcatggtaataataaaaaaaaagtaaaaaagtttattataccaataccaataccaataccaataccaataccaatacaaacaacagtatacaca

SEQ ID NO: 639 - DNA, 120 bp, human papillomavirus
  625339_25353838_333048_HPV31REF.1_1_70
aaacttttttactttttttttattattaccatgcggtccaaacgctctacaaaacgcactaaacgtgcgtctgctacacaattatatcaaacatgtaaagcagcaggtacttgtccatca

SEQ ID NO: 640 - DNA, 120 bp, human papillomavirus
  625339_25353838_333048_HPV31REF.1_1_71
actaccataccttaatatttggtctgcaatggtagtatgttctattttaggtataacgtctgatggacaagtacctgctgctttacatgtttgatataattgtgtagcagacgcacgttt

SEQ ID NO: 641 - DNA, 120 bp, human papillomavirus
  625339_25353838_333048_HPV31REF.1_1_72
gacgttatacctaaaatagaacatactaccattgcagaccaaatattaaggtatggtagtatgggtgttttttttggtgggttgggtattgggtccggctctggtactgggggtcgcact

SEQ ID NO: 642 - DNA, 120 bp, human papillomavirus
  625339_25353838_333048_HPV31REF.1_1_73
tctaataggtatacttgcctcagatactgtagaaggacgtgtactaagagggacatatccagtgcgacccccagtaccagagccggacccaatacccaacccaccaaaaaaaacacccat

SEQ ID NO: 643 - DNA, 120 bp, human papillomavirus
  625339_25353838_333048_HPV31REF.1_1_74
ggatatgtccctcttagtacacgtccttctacagtatctgaggcaagtatacctattagaccaccagttagcattgaccctgtaggtcccttggacccctctatagtaagtcttgttgaa

SEQ ID NO: 644 - DNA, 120 bp, human papillomavirus
  625339_25353838_333048_HPV31REF.1_1_75
agatgttgtaggagggtgtggtataggagcaggggcaccaacatcaacaattccagattcttcaacaagacttactatagaggggtccaagggacctacagggtcaatgctaactggtgg

SEQ ID NO: 645 - DNA, 120 bp, human papillomavirus
  625339_25353838_333048_HPV31REF.1_1_76
gaatctggaattgttgatgttggtgcccctgctcctataccacaccctcctacaacatctgggtttgacattgctacaactgcagacacaacacctgcaattttagatgtaacaagtgtt

SEQ ID NO: 646 - DNA, 120 bp, human papillomavirus
  625339_25353838_333048_HPV31REF.1_1_77
tgcaggtgtaggaggctgcaatacagatggatcagtaaaagtaggattttcatgtgtgctaacacttgttacatctaaaattgcaggtgttgtgtctgcagttgtagcaatgtcaaaccc

SEQ ID NO: 647 - DNA, 120 bp, human papillomavirus
  625339_25353838_333048_HPV31REF.1_1_78
agcacacatgaaaatcctacttttactgatccatctgtattgcagcctcctacacctgcagaaacatcaggtcatttactactttcatcatcatctattagcacacataattatgaggaa

SEQ ID NO: 648 - DNA, 120 bp, human papillomavirus
  625339_25353838_333048_HPV31REF.1_1_79
gggtgtgctacttgttatgttttcattattagtagaaacaataaatgtatccataggtatttcctcataattatgtgtgctaatagatgatgatgaaagtagtaaatgacctgatgtttc

SEQ ID NO: 649 - DNA, 120 bp, human papillomavirus
  625339_25353838_333048_HPV31REF.1_1_80
atacctatggatacatttattgtttctactaataatgaaaacataacaagtagcacacccattccaggggtgcgccgtcctgcacgtttagggttatatagtaaggctacacaacaagta

SEQ ID NO: 650 - DNA, 120 bp, human papillomavirus
  625339_25353838_333048_HPV31REF.1_1_81
agggttttcatatgtaattagctgttttggagcactaagaaacgttggatcaataacttttacttgttgtgtagccttactatataaccctaaacgtgcaggacggcgcacccctggaat

SEQ ID NO: 651 - DNA, 120 bp, human papillomavirus
  625339_25353838_333048_HPV31REF.1_1_82
aaagttattgatccaacgtttcttagtgctccaaaacagctaattacatatgaaaaccctgcctatgaaactgtaaatgctgaagaatctttatacttttccaatacatcgcataatata

SEQ ID NO: 652 - DNA, 120 bp, human papillomavirus
  625339_25353838_333048_HPV31REF.1_1_83
acgtgaggtaagggcaggcctatgtaatgctataatatctagaaagtcgggatcaggggctatattatgcgatgtattggaaaagtataaagattcttcagcatttacagtttcataggc

SEQ ID NO: 653 - DNA, 120 bp, human papillomavirus
  625339_25353838_333048_HPV31REF.1_1_84
gcccctgatcccgactttctagatattatagcattacataggcctgcccttacctcacgtaggaacactgttagatatagtagactaggtaataaacaaactttgcgcactcgtagtggt

SEQ ID NO: 654 - DNA, 120 bp, human papillomavirus
  625339_25353838_333048_HPV31REF.1_1_85
acctgcaggattaatactactaatatcataataataatgcacccttgcaccaatagtagcaccactacgagtgcgcaaagtttgtttattacctagtctactatatctaacagtgttcct

SEQ ID NO: 655 - DNA, 120 bp, human papillomavirus
  625339_25353838_333048_HPV31REF.1_1_86
gctactattggtgcaagggtgcattattattatgatattagtagtattaatcctgcaggtgaaagtattgaaatgcaacctttaggggcgtctgcaactactacttctactttaaatgat

SEQ ID NO: 656 - DNA, 120 bp, human papillomavirus
  625339_25353838_333048_HPV31REF.1_1_87
attatgtgtggcaggtgtatccacagtaaaatcagtgtctgcataaatgtcatataagccatcatttaaagtagaagtagtagttgcagacgcccctaaaggttgcatttcaatactttc

SEQ ID NO: 657 - DNA, 120 bp, human papillomavirus
  625339_25353838_333048_HPV31REF.1_1_88
ggcttatatgacatttatgcagacactgattttactgtggatacacctgccacacataatgtttccccttctactgctgtacagtccacatctgctgtgtctgcctatgtacctacaaat

SEQ ID NO: 658 - DNA, 120 bp, human papillomavirus
  625339_25353838_333048_HPV31REF.1_1_89
aggtacatcaggcccagaaaatatgggaatgtcaaaacctgtacttagtggcacagtggtatttgtaggtacataggcagacacagcagatgtggactgtacagcagtagaaggggaaac

SEQ ID NO: 659 - DNA, 120 bp, human papillomavirus
  625339_25353838_333048_HPV31REF.1_1_90
accactgtgccactaagtacaggttttgacattcccatattttctgggcctgatgtacctatagagcatgcacctacacaggttttcccatttcctttggcccctacaacgccacaagtg

SEQ ID NO: 660 - DNA, 120 bp, human papillomavirus
  625339_25353838_333048_HPV31REF.1_1_91
acgttttaacatataataactagggtgcaaataaaaatcacccccatcaacaaaaatagacacttgtggcgttgtaggggccaaaggaaatgggaaaacctgtgtaggtgcatgctctat

SEQ ID NO: 661 - DNA, 120 bp, human papillomavirus
  625339_25353838_333048_HPV31REF.1_1_92
tctatttttgttgatgggggtgatttttatttgcaccctagttattatatgttaaaacgtcgacgtaaacgtgtatcatatttttttacagatgtctctgtggcggcctagcgaggctac

SEQ ID NO: 662 - DNA, 120 bp, human papillomavirus
  625339_25353838_333048_HPV31REF.1_1_93
cgtgttacatattcatccgtgcttacaactttagacactgggacaggtggtaagtagacagtagcctcgctaggccgccacagagacatctgtaaaaaaatatgatacacgtttacgtcg

SEQ ID NO: 663 - DNA, 120 bp, human papillomavirus
  625339_25353838_333048_HPV31REF.1_1_94
tgtctacttaccacctgtcccagtgtctaaagttgtaagcacggatgaatatgtaacacgaaccaacatatattatcacgcaggcagtgctaggctgcttacagtaggccatccatatta

SEQ ID NO: 664 - DNA, 120 bp, human papillomavirus
  625339_25353838_333048_HPV31REF.1_1_95
tgtaatcctgacacctttggtacaactatttttttaggattgtcagatttaggtatggaataatatggatggcctactgtaagcagcctagcactgcctgcgtgataatatatgttggtt

SEQ ID NO: 665 - DNA, 120 bp, human papillomavirus
  625339_25353838_333048_HPV31REF.1_1_96
ttccatacctaaatctgacaatcctaaaaaaatagttgtaccaaaggtgtcaggattacaatatagggtatttagggttcgtttaccagatccaaacaaatttggatttcctgatacatc

SEQ ID NO: 666 - DNA, 120 bp, human papillomavirus
  625339_25353838_333048_HPV31REF.1_1_97
cgacctacctctaaaccaacacaggcccaaactaagcgttgagtttcaggattataaaaagatgtatcaggaaatccaaatttgtttggatctggtaaacgaaccctaaataccctatat

SEQ ID NO: 667 - DNA, 120 bp, human papillomavirus
  625339_25353838_333048_HPV31REF.1_1_98
tttttataatcctgaaactcaacgcttagtttgggcctgtgttggtttagaggtaggtcgcgggcagccattaggtgtaggtattagtggtcatccattattaaataaatttgatgacac

SEQ ID NO: 668 - DNA, 120 bp, human papillomavirus
  625339_25353838_333048_HPV31REF.1_1_99
attgatatacattccctattatcagtgccaggaccaccggcatatctattagagttttcagtgtcatcaaatttatttaataatggatgaccactaatacctacacctaatggctgcccg

SEQ ID NO: 669 - DNA, 120 bp, human papillomavirus
  625339_25353838_333048_HPV31REF.1_1_100
tgaaaactctaatagatatgccggtggtcctggcactgataatagggaatgtatatcaatggattataaacaaacacaactgtgtttacttggttgcaaaccacctattggagagcattg

SEQ ID NO: 670 - DNA, 120 bp, human papillomavirus
  625339_25353838_333048_HPV31REF.1_1_101
tctaatggaggacaatcaccaggggtaatagcattgttactacaaggactacctttaccccaatgctctccaataggtggtttgcaaccaagtaaacacagttgtgtttgtttataatcc

SEQ ID NO: 671 - DNA, 120 bp, human papillomavirus
  625339_25353838_333048_HPV31REF.1_1_102
gggtaaaggtagtccttgtagtaacaatgctattacccctggtgattgtcctccattagaattaaaaaattcagttatacaagatggggatatggttgatacaggctttggagctatgga

SEQ ID NO: 672 - DNA, 120 bp, human papillomavirus
  625339_25353838_333048_HPV31REF.1_1_103
caaatagaattacaaatgtccaaaggaacattacttttagtgtcttgtaaagcagtaaaatccatagctccaaagcctgtatcaaccatatccccatcttgtataactgaattttttaat

SEQ ID NO: 673 - DNA, 120 bp, human papillomavirus
  625339_25353838_333048_HPV31REF.1_1_104
ttttactgctttacaagacactaaaagtaatgttcctttggacatttgtaattctatttgtaaatatccagattatcttaaaatggttgctgagccatatggcgatacattattttttta

SEQ ID NO: 674 - DNA, 120 bp, human papillomavirus
  625339_25353838_333048_HPV31REF.1_1_105
tcaccaaccgtgcctgatctattaaaaaaatgccttacaaacatttgttccctacgtaaataaaaaaataatgtatcgccatatggctcagcaaccattttaagataatctggatattta

SEQ ID NO: 675 - DNA, 120 bp, human papillomavirus
  625339_25353838_333048_HPV31REF.1_1_106
tttacgtagggaacaaatgtttgtaaggcatttttttaatagatcaggcacggttggtgaatcggtccctactgacttatatattaaaggctccggttcaacagctactttagctaacag

SEQ ID NO: 676 - DNA, 120 bp, human papillomavirus
  625339_25353838_333048_HPV31REF.1_1_107
ttattaaaaatttgtgcatctgaagtaaccatggagccgctaggtgtaggaaagtatgtactgttagctaaagtagctgttgaaccggagcctttaatatataagtcagtagggaccgat

SEQ ID NO: 677 - DNA, 120 bp, human papillomavirus
  625339_25353838_333048_HPV31REF.1_1_108
tacatactttcctacacctagcggctccatggttacttcagatgcacaaatttttaataaaccatattggatgcaacgtgctcagggacacaataatggtatttgttggggcaatcagtt

SEQ ID NO: 678 - DNA, 120 bp, human papillomavirus
  625339_25353838_333048_HPV31REF.1_1_109
gcaattgcagcacaaacagacatattggtactacgtgtggtatctaccacagtaacaaataactgattgccccaacaaataccattattgtgtccctgagcacgttgcatccaatatggt

SEQ ID NO: 679 - DNA, 120 bp, human papillomavirus
  625339_25353838_333048_HPV31REF.1_1_110
atttgttactgtggtagataccacacgtagtaccaatatgtctgtttgtgctgcaattgcaaacagtgatactacatttaaaagtagtaattttaaagagtatttaagacatggtgagga

SEQ ID NO: 680 - DNA, 120 bp, human papillomavirus
  625339_25353838_333048_HPV31REF.1_1_111
gtcattatgtctgcagataatgttattttgcataactgaaatataaattgtaaatcaaattcctcaccatgtcttaaatactctttaaaattactacttttaaatgtagtatcactgttt

SEQ ID NO: 681 - DNA, 120 bp, human papillomavirus
  625339_25353838_333048_HPV31REF.1_1_112
atttgatttacaatttatatttcagttatgcaaaataacattatctgcagacataatgacatatattcacagtatgaatcctgctattttggaagattggaattttggattgaccacacc

SEQ ID NO: 682 - DNA, 120 bp, human papillomavirus
  625339_25353838_333048_HPV31REF.1_1_113
tgacatgtaatggcctgtgaggtgacaaacctataggtatcctccaaagaacctgagggaggtgtggtcaatccaaaattccaatcttccaaaatagcaggattcatactgtgaatatat

SEQ ID NO: 683 - DNA, 120 bp, human papillomavirus
  625339_25353838_333048_HPV31REF.1_1_114
tccctcaggttctttggaggatacctataggtttgtcacctcacaggccattacatgtcaaaaaactgccccccaaaagcccaaggaagatccatttaaagattatgtattttgggaggt

SEQ ID NO: 684 - DNA, 120 bp, human papillomavirus
  625339_25353838_333048_HPV31REF.1_1_115
aaaaatttgcgacccagtggaaactgatctaaatctgcagaaaacttttcttttaaattaacctcccaaaatacataatctttaaatggatcttccttgggcttttggggggcagttttt

SEQ ID NO: 685 - DNA, 120 bp, human papillomavirus
  625339_25353838_333048_HPV31REF.1_1_116
taatttaaaagaaaagttttctgcagatttagatcagtttccactgggtcgcaaatttttattacaggcaggatatagggcacgtcctaaatttaaagcaggtaaacgtagtgcaccctc

SEQ ID NO: 686 - DNA, 120 bp, human papillomavirus
  625339_25353838_333048_HPV31REF.1_1_117
tacatacacatccattactttttagtttttttacgttttgctggtgtagtggtagatgctgagggtgcactacgtttacctgctttaaatttaggacgtgccctatatcctgcctgtaat

SEQ ID NO: 687 - DNA, 120 bp, human papillomavirus
  625339_25353838_333048_HPV31REF.1_1_118
agcatctaccactacaccagcaaaacgtaaaaaaactaaaaagtaatggatgtgtatgtaatacatgtgtctgtatgtgtatgtgcttgtgctgtattgtatatgtgtgtgtttgtgtgt

SEQ ID NO: 688 - DNA, 120 bp, human papillomavirus
  625339_25353838_333048_HPV31REF.1_1_119
aacatactatacatatatacaagtacacacgcatacataaacatacatataccatatataacacacaaacacacacatatacaatacagcacaagcacatacacatacagacacatgtat

SEQ ID NO: 689 - DNA, 120 bp, human papillomavirus
  625339_25353838_333048_HPV31REF.1_1_120
tatatatggtatatgtatgtttatgtatgcgtgtgtacttgtatatatgtatagtatgttatgtgtgtatgtatgctatgtatgttaataaatatgtgtatacctgtgtgtgttgtgtat

SEQ ID NO: 690 - DNA, 120 bp, human papillomavirus
  625339_25353838_333048_HPV31REF.1_1_121
ggaacaatagtttataaaatagtaatagtatgttactaatagggtgtatataaggacaacatacacaacacacacaggtatacacatatttattaacatacatagcatacatacacacat

SEQ ID NO: 691 - DNA, 120 bp, human papillomavirus
  625339_25353838_333048_HPV31REF.1_1_122
gttgtccttatatacaccctattagtaacatactattactattttataaactattgttcctacttgttcctacttgttcctgctcctcccaatagtcatgtacttatttctgcctataat

SEQ ID NO: 692 - DNA, 120 bp, human papillomavirus
  625339_25353838_333048_HPV31REF.1_1_123
gtagctttagttgcaaaaaacggaccgggtgtacaacttttactatggcgtgacacctaaattataggcagaaataagtacatgactattgggaggagcaggaacaagtaggaacaagta

SEQ ID NO: 693 - DNA, 120 bp, human papillomavirus
  625339_25353838_333048_HPV31REF.1_1_124
ttaggtgtcacgccatagtaaaagttgtacacccggtccgttttttgcaactaaagctactccattttgattttatgcagccattttaaatccctaaccgttttcggttgcattgtttaa

SEQ ID NO: 694 - DNA, 120 bp, human papillomavirus
  625339_25353838_333048_HPV31REF.1_1_125
tagtattcaggaaaccaaaaaccgcagaactactgcatcagcatagttgtactagcatgtttaaacaatgcaaccgaaaacggttagggatttaaaatggctgcataaaatcaaaatgga

SEQ ID NO: 695 - DNA, 120 bp, human papillomavirus
  625339_25353838_333048_HPV31REF.1_1_126
acatgctagtacaactatgctgatgcagtagttctgcggtttttggtttcctgaatactagtttttgccaacattctggcttgtagtttcctgcctaacacaccttgccaacatataatc

SEQ ID NO: 696 - DNA, 120 bp, human papillomavirus
  625339_25353838_333048_HPV31REF.1_1_127
gttgaactacagttgtatttaaacaaacatgattcatagtataattgcaaagttggactggattatatgttggcaaggtgtgttaggcaggaaactacaagccagaatgttggcaaaaac

SEQ ID NO: 697 - DNA, 120 bp, human papillomavirus
  625339_25353838_333048_HPV31REF.1_1_128
cagtccaactttgcaattatactatgaatcatgtttgtttaaatacaactgtagttcaactatgtgtcatgcacatatattatattatcctacacaccttaaactgcttttaggcacata

SEQ ID NO: 698 - DNA, 120 bp, human papillomavirus
  625339_25353838_333048_HPV31REF.1_1_129
ggcagtttaaacatgtgcaaaagccagcactgcaatcaaggatatagataatctacaaaatatgtgcctaaaagcagtttaaggtgtgtaggataatataatatatgtgcatgacacata

SEQ ID NO: 699 - DNA, 120 bp, human papillomavirus
  625339_25353838_333048_HPV31REF.1_1_130
ttttgtagattatctatatccttgattgcagtgctggcttttgcacatgtttaaactgccaaggttgtgtcatgcattataaataagttgtatgttactcatataattaattgcatatag

SEQ ID NO: 700 - DNA, 112 bp, human papillomavirus
  625339_25353838_333048_HPV31REF.1_1_131
agtataaaaagaacaattgcttgtaaaactgtaaccgaaaacggtgtaatacctatatgcaattaattatatgagtaacatacaacttatttataatgcatgacacaacctt

SEQ ID NO: 701 - DNA, 120 bp, human papillomavirus; identical to SEQ ID NO: 115
  625339_25353839_333049_HPV33REF.1_1_1

SEQ ID NO: 702 - DNA, 120 bp, human papillomavirus
  625339_25353839_333049_HPV33REF.1_1_2
gtgtatagttgtctccaatgcttggcacaaatcatgcaatgttcgtggtttttcctcagtgtcttgaaacatagtcgtgcagtaccttactgcaaaatgtttgctttatatatgcaccgt

SEQ ID NO: 703 - DNA, 120 bp, human papillomavirus
  625339_25353839_333049_HPV33REF.1_1_3
actgaggaaaaaccacgaacattgcatgatttgtgccaagcattggagacaactatacacaacattgaactacagtgcgtggaatgcaaaaaacctttgcaacgatctgaggtatatgat

SEQ ID NO: 704 - DNA, 120 bp, human papillomavirus
  625339_25353839_333049_HPV33REF.1_1_4
tttacatattccaaatggatttccctctctatatacaactgttaaatctgcaaatgcaaaatcatatacctcagatcgttgcaaaggttttttgcattccacgcactgtagttcaatgtt

SEQ ID NO: 705 - DNA, 120 bp, human papillomavirus
  625339_25353839_333049_HPV33REF.1_1_5
tttgcatttgcagatttaacagttgtatatagagagggaaatccatttggaatatgtaaactgtgtttgcggttcttatctaaaattagtgaatatagacattataattattctgtatat

SEQ ID NO: 706 - DNA, 120 bp, human papillomavirus
  625339_25353839_333049_HPV33REF.1_1_6
aatacacctaattaatatttcatttaaaggttttttaactgtttgttctaatgtatttccatatacagaataattataatgtctatattcactaattttagataagaaccgcaaacacag

SEQ ID NO: 707 - DNA, 120 bp, human papillomavirus
  625339_25353839_333049_HPV33REF.1_1_7
ggaaatacattagaacaaacagttaaaaaacctttaaatgaaatattaattaggtgtattatatgtcaaagacctttgtgtcctcaagaaaaaaaacgacatgtggatttaaacaaacga

SEQ ID NO: 708 - DNA, 120 bp, human papillomavirus
  625339_25353839_333049_HPV33REF.1_1_8
acgtcgggacctccaacacgccgcacagcgccctgcccaacgacccgaaatattatgaaatcgtttgtttaaatccacatgtcgttttttttcttgaggacacaaaggtctttgacatat

SEQ ID NO: 709 - DNA, 120 bp, human papillomavirus
  625339_25353839_333049_HPV33REF.1_1_9
tttcataatatttcgggtcgttgggcagggcgctgtgcggcgtgttggaggtcccgacgtagagaaactgcactgtgacgtgtaaaaacgccatgagaggacacaagccaacgttaaagg

SEQ ID NO: 710 - DNA, 120 bp, human papillomavirus
  625339_25353839_333049_HPV33REF.1_1_10
cacttaattgctcatagcagtataggtcagttggttcaggatataaatctaaaacatattcctttaacgttggcttgtgtcctctcatggcgtttttacacgtcacagtgcagtttctct

SEQ ID NO: 711 - DNA, 120 bp, human papillomavirus
  625339_25353839_333049_HPV33REF.1_1_11
aatatgttttagatttatatcctgaaccaactgacctatactgctatgagcaattaagtgacagctcagatgaggatgaaggcttggaccggccagatggacaagcacaaccagccacag

SEQ ID NO: 712 - DNA, 120 bp, human papillomavirus
  625339_25353839_333049_HPV33REF.1_1_12
tgacacataaacgaactgtggtgttacaagtgtgacaacaggttacaatgtagtaatcagctgtggctggttgtgcttgtccatctggccggtccaagccttcatcctcatctgagctgt

SEQ ID NO: 713 - DNA, 120 bp, human papillomavirus
  625339_25353839_333049_HPV33REF.1_1_13
ctgattactacattgtaacctgttgtcacacttgtaacaccacagttcgtttatgtgtcaacagtacagcaagtgacctacgaaccatacagcaactacttatgggcacagtgaatattg

SEQ ID NO: 714 - DNA, 120 bp, human papillomavirus
  625339_25353839_333049_HPV33REF.1_1_14
ttgtaccttcaggatcggccattgtagatgatgtttattgttgtgcacaggtagggcacacaatattcactgtgcccataagtagttgctgtatggttcgtaggtcacttgctgtactgt

SEQ ID NO: 715 - DNA, 120 bp, human papillomavirus
  625339_25353839_333049_HPV33REF.1_1_15
tgtgccctacctgtgcacaacaataaacatcatctacaatggccgatcctgaaggtacaaatggggctgggatggggtgtactggttggtttgaggtagaagcagtcatagagagaagaa

SEQ ID NO: 716 - DNA, 120 bp, human papillomavirus
  625339_25353839_333049_HPV33REF.1_1_16
gtaaatccgtgccactgtcatctgctgtttcatcctcatcttctgaaatattatctcctgttcttctctctatgactgcttctacctcaaaccaaccagtacaccccatcccagccccat

SEQ ID NO: 717 - DNA, 120 bp, human papillomavirus
  625339_25353839_333049_HPV33REF.1_1_17
caggagataatatttcagaagatgaggatgaaacagcagatgacagtggcacggatttactagagtttatagatgattctatggaaaatagtatacaggcagacacagaggcagcccggg

SEQ ID NO: 718 - DNA, 120 bp, human papillomavirus
  625339_25353839_333049_HPV33REF.1_1_18
ttcgttttagtgcacacacagcatttaaatcatcctccccttcctgtatattaaacaatgcccgggctgcctctgtgtctgcctgtatactattttccatagaatcatctataaactcta

SEQ ID NO: 719 - DNA, 120 bp, human papillomavirus
  625339_25353839_333049_HPV33REF.1_1_19
cattgtttaatatacaggaaggggaggatgatttaaatgctgtgtgtgcactaaaacgaaagtttgccgcatgttcacaaagtgctgcggaggacgttgttgatcgtgctgcaaacccgt

SEQ ID NO: 720 - DNA, 120 bp, human papillomavirus
  625339_25353839_333049_HPV33REF.1_1_20
gctcatctatttttcgttttctgtatgtgcattctttatttttattaatagacgttctacacgggtttgcagcacgatcaacaacgtcctccgcagcactttgtgaacatgcggcaaact

SEQ ID NO: 721 - DNA, 120 bp, human papillomavirus
  625339_25353839_333049_HPV33REF.1_1_21
gtagaacgtctattaataaaaataaagaatgcacatacagaaaacgaaaaatagatgagctagaagacagcggatatggcaatactgaagtggaaactcagcagatggtacaacaggtag

SEQ ID NO: 722 - DNA, 120 bp, human papillomavirus
  625339_25353839_333049_HPV33REF.1_1_22
aatcatcccccaccccactagattctaagtcatttaagtttgtgtcgccattttgactttctacctgttgtaccatctgctgagtttccacttcagtattgccatatccgctgtcttcta

SEQ ID NO: 723 - DNA, 120 bp, human papillomavirus
  625339_25353839_333049_HPV33REF.1_1_23
aaagtcaaaatggcgacacaaacttaaatgacttagaatctagtggggtgggggatgattcagaagtaagctgtgagacaaatgtagatagctgtgaaaatgttacgttgcaggaaatta

SEQ ID NO: 724 - DNA, 120 bp, human papillomavirus
  625339_25353839_333049_HPV33REF.1_1_24
aggcctctttaaatttatataatatatttgcttttgtattactactatgtagaacattactaatttcctgcaacgtaacattttcacagctatctacatttgtctcacagcttacttctg

SEQ ID NO: 725 - DNA, 120 bp, human papillomavirus
  625339_25353839_333049_HPV33REF.1_1_25
gtaatgttctacatagtagtaatacaaaagcaaatatattatataaatttaaagaggcctatggaataagttttatggaattagtaagaccatttaaaagtgataaaacaagctgtacag

SEQ ID NO: 726 - DNA, 120 bp, human papillomavirus
  625339_25353839_333049_HPV33REF.1_1_26
ttaatacttttaaactttctgctactgatggactaattccatatcctgttatacaccaatctgtacagcttgttttatcacttttaaatggtcttactaattccataaaacttattccat

SEQ ID NO: 727 - DNA, 120 bp, human papillomavirus
  625339_25353839_333049_HPV33REF.1_1_27
attggtgtataacaggatatggaattagtccatcagtagcagaaagtttaaaagtattaattaaacagcatagtttgtatactcatttacaatgtttaacttgcgatagaggaataataa

SEQ ID NO: 728 - DNA, 120 bp, human papillomavirus
  625339_25353839_333049_HPV33REF.1_1_28
tcattagttttgctactgttaacctgtttttgctacacctaaatctaattaacaataatattattattcctctatcgcaagttaaacattgtaaatgagtatacaaactatgctgtttaa

SEQ ID NO: 729 - DNA, 120 bp, human papillomavirus
  625339_25353839_333049_HPV33REF.1_1_29
tattattgttaattagatttaggtgtagcaaaaacaggttaacagtagcaaaactaatgagtaatttattatcaatacctgaaacatgtatggttatagagccaccaaaattacggagcc

SEQ ID NO: 730 - DNA, 120 bp, human papillomavirus
  625339_25353839_333049_HPV33REF.1_1_30
taccttgtacatcactaatgtttgacattgctgttctaaaccaatacaatgcacatgtttggctccgtaattttggtggctctataaccatacatgtttcaggtattgataataaattac

SEQ ID NO: 731 - DNA, 120 bp, human papillomavirus
  625339_25353839_333049_HPV33REF.1_1_31
aaacatgtgcattgtattggtttagaacagcaatgtcaaacattagtgatgtacaaggtacaacacctgaatggatagatagactaactgttttacaacatagctttaatgataatatat

SEQ ID NO: 732 - DNA, 120 bp, human papillomavirus
  625339_25353839_333049_HPV33REF.1_1_32
tgtcactatcgtccgttaactcgttatcatatgcccactgtaccatttcacttaaatcaaatatattatcattaaagctatgttgtaaaacagttagtctatctatccattcaggtgttg

SEQ ID NO: 733 - DNA, 120 bp, human papillomavirus
  625339_25353839_333049_HPV33REF.1_1_33
ttgatttaagtgaaatggtacagtgggcatatgataacgagttaacggacgatagtgacattgcatattattatgcacaacttgcagattcaaatagtaatgctgctgcatttttaaaaa

SEQ ID NO: 734 - DNA, 120 bp, human papillomavirus
  625339_25353839_333049_HPV33REF.1_1_34
cttttttataatgtctacacattattccacagtcctttactatttttgcttgtgagttactttttaaaaatgcagcagcattactatttgaatctgcaagttgtgcataataatatgcaa

SEQ ID NO: 735 - DNA, 120 bp, human papillomavirus
  625339_25353839_333049_HPV33REF.1_1_35
gtaactcacaagcaaaaatagtaaaggactgtggaataatgtgtagacattataaaaaagcagaaaaacgtaaaatgtcaataggacaatggatacaaagtagatgtgaaaaaacaaatg

SEQ ID NO: 736 - DNA, 120 bp, human papillomavirus
  625339_25353839_333049_HPV33REF.1_1_36
ctgtaaattcaatgttttgatatcttaacaactgtactattggtctccaatttcctccatcatttgttttttcacatctactttgtatccattgtcctattgacattttacgtttttctg

SEQ ID NO: 737 - DNA, 120 bp, human papillomavirus
  625339_25353839_333049_HPV33REF.1_1_37
atggaggaaattggagaccaatagtacagttgttaagatatcaaaacattgaatttacagcatttttaggtgcatttaaaaagtttttaaaaggtataccaaaaaaaagctgtatgctaa

SEQ ID NO: 738 - DNA, 120 bp, human papillomavirus
  625339_25353839_333049_HPV33REF.1_1_38
ttaaaaactgtattaaactcattccaaaatatgactttcctgtatttgctggtccacaaattagcatacagcttttttttggtataccttttaaaaactttttaaatgcacctaaaaatg

SEQ ID NO: 739 - DNA, 120 bp, human papillomavirus
  625339_25353839_333049_HPV33REF.1_1_39
tttgtggaccagcaaatacaggaaagtcatattttggaatgagtttaatacagtttttaaaagggtgtgttatatcatgtgtaaattctaaaagtcacttttggttgcagccattatcag

SEQ ID NO: 740 - DNA, 120 bp, human papillomavirus
  625339_25353839_333049_HPV33REF.1_1_40
aatcatctatatatgtccaacttattggcgttacatcatctatcattcctatttttgcatctgataatggctgcaaccaaaagtgacttttagaatttacacatgatataacacaccctt

SEQ ID NO: 741 - DNA, 120 bp, human papillomavirus
  625339_25353839_333049_HPV33REF.1_1_41
atgcaaaaataggaatgatagatgatgtaacgccaataagttggacatatatagatgattacatgagaaatgcgttagatggaaatgaaatttcaatagatgtgaaacatagggcattag

SEQ ID NO: 742 - DNA, 120 bp, human papillomavirus
  625339_25353839_333049_HPV33REF.1_1_42
tagagtctgtgcctgcatttgtatttgaggtaagaagcagtggtggacattttaattgcactaatgccctatgtttcacatctattgaaatttcatttccatctaacgcatttctcatgt

SEQ ID NO: 743 - DNA, 120 bp, human papillomavirus
  625339_25353839_333049_HPV33REF.1_1_43
tgcaattaaaatgtccaccactgcttcttacctcaaatacaaatgcaggcacagactctagatggccatatttacatagtagattaacagtatttgaatttaaaaatccattcccatttg

SEQ ID NO: 744 - DNA, 120 bp, human papillomavirus
  625339_25353839_333049_HPV33REF.1_1_44
ttgagaaaaaggatttccaattttcatcatttattgcatacactgggttaccattttcatcaaatgggaatggatttttaaattcaaatactgttaatctactatgtaaatatggccatc

SEQ ID NO: 745 - DNA, 120 bp, human papillomavirus
  625339_25353839_333049_HPV33REF.1_1_45
atgaaaatggtaacccagtgtatgcaataaatgatgaaaattggaaatcctttttctcaaggacgtggtgcaaattagatttaatagaggaagaggacaaggaaaaccatggaggaaata

SEQ ID NO: 746 - DNA, 120 bp, human papillomavirus
  625339_25353839_333049_HPV33REF.1_1_46
agttttatcagcttcgtaaagatctagtattttctcctgcactgcatttaaacgtgctgatatttcctccatggttttccttgtcctcttcctctattaaatctaatttgcaccacgtcc

SEQ ID NO: 747 - DNA, 120 bp, human papillomavirus
  625339_25353839_333049_HPV33REF.1_1_47
tcagcacgtttaaatgcagtgcaggagaaaatactagatctttacgaagctgataaaactgatttaccatcacaaattgaacattggaaactgatacgcatggagtgtgctttattgtat

SEQ ID NO: 748 - DNA, 120 bp, human papillomavirus
  625339_25353839_333049_HPV33REF.1_1_48
tgctaacaaagaaggcaccacctggtggcataaatgtgaaaatcccatttgtttggctgtatacaataaagcacactccatgcgtatcagtttccaatgttcaatttgtgatggtaaatc

SEQ ID NO: 749 - DNA, 120 bp, human papillomavirus
  625339_25353839_333049_HPV33REF.1_1_49
acagccaaacaaatgggattttcacatttatgccaccaggtggtgccttctttgttagcatcaaagaccaaagcatttcaagtaattgaactacaaatggcattagagacattaagtaaa

SEQ ID NO: 750 - DNA, 120 bp, human papillomavirus
  625339_25353839_333049_HPV33REF.1_1_50
acaaagccacacctctaagcttgtttgttgcaatgtccattggcttgtactatactgtgatttacttaatgtctctaatgccatttgtagttcaattacttgaaatgctttggtctttga

SEQ ID NO: 751 - DNA, 120 bp, human papillomavirus
  625339_25353839_333049_HPV33REF.1_1_51
tcacagtatagtacaagccaatggacattgcaacaaacaagcttagaggtgtggctttgtgaaccaccaaaatgttttaaaaaacaaggagaaacagtaactgtgcaatatgacaatgac

SEQ ID NO: 752 - DNA, 120 bp, human papillomavirus
  625339_25353839_333049_HPV33REF.1_1_52
tgtatcttcctctataatatatatttcaccccagtttgtataatccattgtattttttttgtcattgtcatattgcacagttactgtttctccttgttttttaaaacattttggtggttc

SEQ ID NO: 753 - DNA, 120 bp, human papillomavirus
  625339_25353839_333049_HPV33REF.1_1_53
aaaaaaaatacaatggattatacaaactggggtgaaatatatattatagaggaagatacatgtactatggttacagggaaagtagattatataggtatgtattatatacataactgtgaa

SEQ ID NO: 754 - DNA, 120 bp, human papillomavirus
  625339_25353839_333049_HPV33REF.1_1_54
ccacatttgtgttttagaatactttgcagcatcctctttaaaatatttaaaatataccttttcacagttatgtatataatacatacctatataatctactttccctgtaaccatagtaca

SEQ ID NO: 755 - DNA, 120 bp, human papillomavirus
  625339_25353839_333049_HPV33REF.1_1_55
aaggtatattttaaatattttaaagaggatgctgcaaagtattctaaaacacaaatgtgggaagtacatgtgggtggtcaggtaattgtttgtcctacgtctatatctagcaaccaaata

SEQ ID NO: 756 - DNA, 120 bp, human papillomavirus
  625339_25353839_333049_HPV33REF.1_1_56
cgctgcttgtggtggtcggttatcgttgtctgtctgtatgtcagcagtttcagtagtggatatttggttgctagatatagacgtaggacaaacaattacctgaccacccacatgtacttc

SEQ ID NO: 757 - DNA, 120 bp, human papillomavirus
  625339_25353839_333049_HPV33REF.1_1_57
tccactactgaaactgctgacatacagacagacaacgataaccgaccaccacaagcagcggccaaacgacgacgacctgcagacaccacagacaccgcccagccccttacaaagctgttc

SEQ ID NO: 758 - DNA, 120 bp, human papillomavirus
  625339_25353839_333049_HPV33REF.1_1_58
cttgtttgtgcagttagttgcagtacgtgctgttctattgtccaaggcggggtctgcacagaacagctttgtaaggggctgggcggtgtctgtggtgtctgcaggtcgtcgtcgtttggc

SEQ ID NO: 759 - DNA, 120 bp, human papillomavirus
  625339_25353839_333049_HPV33REF.1_1_59
tgtgcagaccccgccttggacaatagaacagcacgtactgcaactaactgcacaaacaagcagcggactgtgtgtagttctaacgttgcacctatagtgcatttaaaaggtgaatcaaat

SEQ ID NO: 760 - DNA, 120 bp, human papillomavirus
  625339_25353839_333049_HPV33REF.1_1_60
tgacatagaactatacaactctttataaggttttaatctgtatcttaaacattttaaactatttgattcaccttttaaatgcactataggtgcaacgttagaactacacacagtccgctg

SEQ ID NO: 761 - DNA, 120 bp, human papillomavirus
  625339_25353839_333049_HPV33REF.1_1_61
agtttaaaatgtttaagatacagattaaaaccttataaagagttgtatagttctatgtcatccacctggcattggaccagtgacaacaaaaatagtaaaaatggaattgtaactgtaaca

SEQ ID NO: 762 - DNA, 120 bp, human papillomavirus
  625339_25353839_333049_HPV33REF.1_1_62
cacagtaggtggtatttttacggtacctaaaaacatttgttgttgctgttcagttacaaatgttacagttacaattccatttttactatttttgttgtcactggtccaatgccaggtgga

SEQ ID NO: 763 - DNA, 120 bp, human papillomavirus
  625339_25353839_333049_HPV33REF.1_1_63
tttgtaactgaacagcaacaacaaatgtttttaggtaccgtaaaaataccacctactgtgcaaataagtactggatttatgacattataagtgtacatcacaagccaatatgtgctgcta

SEQ ID NO: 764 - DNA, 120 bp, human papillomavirus
  625339_25353839_333049_HPV33REF.1_1_64
gataagcataaaaacaatataaaacataatacaaaaacaaatatcatggttatatacaattagcagcacatattggcttgtgatgtacacttataatgtcataaatccagtacttatttg

SEQ ID NO: 765 - DNA, 120 bp, human papillomavirus
  625339_25353839_333049_HPV33REF.1_1_65
attgtatataaccatgatatttgtttttgtattatgttttatattgtttttatgcttatccttattattacgtcctttaatactttccatttctacctatgcttggttgctggtgttggt

SEQ ID NO: 766 - DNA, 120 bp, human papillomavirus
  625339_25353839_333049_HPV33REF.1_1_66
aacaacaaatagcaaaaaaaaatttttaaaggagatcccacaaacacccaaagcagcaataccaacaccagcaaccaagcataggtagaaatggaaagtattaaaggacgtaataataag

SEQ ID NO: 767 - DNA, 120 bp, human papillomavirus
  625339_25353839_333049_HPV33REF.1_1_67
attgctgctttgggtgtttgtgggatctcctttaaaaatttttttttgctatttgttgtttttatatttaccaatgatgtgtattaattttcatgcacagcatatgacacaacaagagta

SEQ ID NO: 768 - DNA, 120 bp, human papillomavirus
  625339_25353839_333049_HPV33REF.1_1_68
acaatgttaaaacaccaccatgtgcacatatatacaaacaatatatacatgtatatacattactcttgttgtgtcatatgctgtgcatgaaaattaatacacatcattggtaaatataaa

SEQ ID NO: 769 - DNA, 120 bp, human papillomavirus
  625339_25353839_333049_HPV33REF.1_1_69
atgtatatacatgtatatattgtttgtatatatgtgcacatggtggtgttttaacattgttgttgttattttagtttttttttttttgtattactaataaatacctttatattttagcag

SEQ ID NO: 770 - DNA, 120 bp, human papillomavirus
  625339_25353839_333049_HPV33REF.1_1_70
tagttgtgttgcagatgcacgcttgcgccttgtagatcgtttgtgtctcataataatacactgctaaaatataaaggtatttattagtaatacaaaaaaaaaaaaactaaaataacaaca

SEQ ID NO: 771 - DNA, 120 bp, human papillomavirus
  625339_25353839_333049_HPV33REF.1_1_71
tgtattattatgagacacaaacgatctacaaggcgcaagcgtgcatctgcaacacaactataccaaacatgcaaggccacaggcacctgcccacccgatgttattcctaaagtggaagga

SEQ ID NO: 772 - DNA, 120 bp, human papillomavirus
  625339_25353839_333049_HPV33REF.1_1_72
taaaccaccaaaaaaaacccctaaactgccatatttaagaatttgatctgctatggtacttccttccactttaggaataacatcgggtgggcaggtgcctgtggccttgcatgtttggta

SEQ ID NO: 773 - DNA, 120 bp, human papillomavirus
  625339_25353839_333049_HPV33REF.1_1_73
agtaccatagcagatcaaattcttaaatatggcagtttaggggttttttttggtggtttaggtattggcacaggctctggttcaggtggaaggactggctatgtacctattggtactgac

SEQ ID NO: 774 - DNA, 120 bp, human papillomavirus
  625339_25353839_333049_HPV33REF.1_1_74
aacagtgtctacagtaaccggaggacgtataggctgcaaggggattgcagctgtaggtgggtcagtaccaataggtacatagccagtccttccacctgaaccagagcctgtgccaatacc

SEQ ID NO: 775 - DNA, 120 bp, human papillomavirus
  625339_25353839_333049_HPV33REF.1_1_75
ccacctacagctgcaatccccttgcagcctatacgtcctccggttactgtagacactgttggacctttagactcgtctatagtgtcattaatagaagaaacaagttttatagaggcaggt

SEQ ID NO: 776 - DNA, 120 bp, human papillomavirus
  625339_25353839_333049_HPV33REF.1_1_76
agtatctgcagatgtagtaacatcaaaacctgatggtgtaggaatagatggggctggtgcacctgcctctataaaacttgtttcttctattaatgacactatagacgagtctaaaggtcc

SEQ ID NO: 777 - DNA, 120 bp, human papillomavirus
  625339_25353839_333049_HPV33REF.1_1_77
gcaccagccccatctattcctacaccatcaggttttgatgttactacatctgcagatactacacctgcaattattaatgtttcatctgttggggagtcatctattcaaactatttctaca

SEQ ID NO: 778 - DNA, 120 bp, human papillomavirus
  625339_25353839_333049_HPV33REF.1_1_78
ggcttctgcaggcgctggagggtgtagtacagatggttcagtaaatgtgggatttaaatgtgtagaaatagtttgaatagatgactccccaacagatgaaacattaataattgcaggtgt

SEQ ID NO: 779 - DNA, 120 bp, human papillomavirus
  625339_25353839_333049_HPV33REF.1_1_79
catttaaatcccacatttactgaaccatctgtactacaccctccagcgcctgcagaagcctctggacattttatattttcttcccctactgttagcacacaaagttatgaaaacatacca

SEQ ID NO: 780 - DNA, 120 bp, human papillomavirus
  625339_25353839_333049_HPV33REF.1_1_80
tggaatgggcgtgcttgatgttacattactactgtctgtggaaacaacaaaggtatccattggtatgttttcataactttgtgtgctaacagtaggggaagaaaatataaaatgtccaga

SEQ ID NO: 781 - DNA, 120 bp, human papillomavirus
  625339_25353839_333049_HPV33REF.1_1_81
atggatacctttgttgtttccacagacagtagtaatgtaacatcaagcacgcccattccagggtctcgccctgtggcacgccttggtttatatagtcgcaatacccaacaggttaaggtt

SEQ ID NO: 782 - DNA, 120 bp, human papillomavirus
  625339_25353839_333049_HPV33REF.1_1_82
aaatgcaggattatcatatgttataagtttatgaggcgatgttaaaaaagcagggtcaacaaccttaacctgttgggtattgcgactatataaaccaaggcgtgccacagggcgagaccc

SEQ ID NO: 783 - DNA, 120 bp, human papillomavirus
  625339_25353839_333049_HPV33REF.1_1_83
gttgaccctgcttttttaacatcgcctcataaacttataacatatgataatcctgcatttgaaagctttgaccctgaagacacattacaatttcaacatagtgatatatcacctgctcct

SEQ ID NO: 784 - DNA, 120 bp, human papillomavirus
  625339_25353839_333049_HPV33REF.1_1_84
atgtctacgagatgtaatagcaggcctatgtaatgcaataatatctagaaagtcaggatcaggagcaggtgatatatcactatgttgaaattgtaatgtgtcttcagggtcaaagctttc

SEQ ID NO: 785 - DNA, 120 bp, human papillomavirus
  625339_25353839_333049_HPV33REF.1_1_85
gatcctgactttctagatattattgcattacataggcctgctattacatctcgtagacatactgtgcgttttagtagagtaggtcaaaaagccacacttaaaactcgcagtggtaaacaa

SEQ ID NO: 786 - DNA, 120 bp, human papillomavirus
  625339_25353839_333049_HPV33REF.1_1_86
ggtgtggtctaaaggcacaataggacttaaatcctgataataatgtattctagctccaatttgtttaccactgcgagttttaagtgtggctttttgacctactctactaaaacgcacagt

SEQ ID NO: 787 - DNA, 120 bp, human papillomavirus
  625339_25353839_333049_HPV33REF.1_1_87
attggagctagaatacattattatcaggatttaagtcctattgtgcctttagaccacaccgtgccaaatgaacaatatgaattacagcctttacatgatacttctacatcgtcttatagt

SEQ ID NO: 788 - DNA, 120 bp, human papillomavirus
  625339_25353839_333049_HPV33REF.1_1_88
cattggggtgtgtacattatccacatcgtcagcataaacatcatacaaaccatcattaatactataagacgatgtagaagtatcatgtaaaggctgtaattcatattgttcatttggcac

SEQ ID NO: 789 - DNA, 120 bp, human papillomavirus
  625339_25353839_333049_HPV33REF.1_1_89
attaatgatggtttgtatgatgtttatgctgacgatgtggataatgtacacaccccaatgcaacactcatacagtacgtttgcaacaacacgtaccagcaatgtgtctatacctttaaat

SEQ ID NO: 790 - DNA, 120 bp, human papillomavirus
  625339_25353839_333049_HPV33REF.1_1_90
tgtgggaaataaaggggaaggtatatcagggccagacataacaggagtatcaaatcctgtatttaaaggtatagacacattgctggtacgtgttgttgcaaacgtactgtatgagtgttg

SEQ ID NO: 791 - DNA, 120 bp, human papillomavirus
  625339_25353839_333049_HPV33REF.1_1_91
acaggatttgatactcctgttatgtctggccctgatataccttcccctttatttcccacatctagcccatttgttcctatttcgcctttttttccttttgacaccattgttgtagacggt

SEQ ID NO: 792 - DNA, 120 bp, human papillomavirus
  625339_25353839_333049_HPV33REF.1_1_92
tggaaaacgtttacgcctgcgacgtaaaataaaataactaggatgtaaaacaaagtcagcaccgtctacaacaatggtgtcaaaaggaaaaaaaggcgaaataggaacaaatgggctaga

SEQ ID NO: 793 - DNA, 120 bp, human papillomavirus
  625339_25353839_333049_HPV33REF.1_1_93
gctgactttgttttacatcctagttattttattttacgtcgcaggcgtaaacgttttccatatttttttacagatgtccgtgtggcggcctagtgaggccacagtgtacctgcctcctgt

SEQ ID NO: 794 - DNA, 120 bp, human papillomavirus
  625339_25353839_333049_HPV33REF.1_1_94
taataataaatgcttgtgcgagacacatattcatcagtgctgacaactttagatacaggtacaggaggcaggtacactgtggcctcactaggccgccacacggacatctgtaaaaaaata

SEQ ID NO: 795 - DNA, 120 bp, human papillomavirus
  625339_25353839_333049_HPV33REF.1_1_95
acctgtatctaaagttgtcagcactgatgaatatgtgtctcgcacaagcatttattattatgctggtagttccagacttcttgctgttggccatccatatttttctattaaaaatcctac

SEQ ID NO: 796 - DNA, 120 bp, human papillomavirus
  625339_25353839_333049_HPV33REF.1_1_96
accctaaaaaccctatattgcaagcctgatactttgggtaccaataattttttagcgttagtaggatttttaatagaaaaatatggatggccaacagcaagaagtctggaactaccagca

SEQ ID NO: 797 - DNA, 120 bp, human papillomavirus
  625339_25353839_333049_HPV33REF.1_1_97
taacgctaaaaaattattggtacccaaagtatcaggcttgcaatatagggtttttagggtccgtttaccagatcctaataaatttggatttcctgacacctccttttataaccctgatac

SEQ ID NO: 798 - DNA, 120 bp, human papillomavirus
  625339_25353839_333049_HPV33REF.1_1_98
acgcctaatggctgccctctacctatttcaaggcctacacatgcccatactaatcgttgtgtatcagggttataaaaggaggtgtcaggaaatccaaatttattaggatctggtaaacgg

SEQ ID NO: 799 - DNA, 120 bp, human papillomavirus
  625339_25353839_333049_HPV33REF.1_1_99
acaacgattagtatgggcatgtgtaggccttgaaataggtagagggcagccattaggcgttggcataagtggtcatcctttattaaacaaatttgatgacactgaaaccggtaacaagta

SEQ ID NO: 800 - DNA, 120 bp, human papillomavirus
  625339_25353839_333049_HPV33REF.1_1_100
tgtgtttgtttataatccatggataaacattccctattatcagcacccggttgtccaggatacttgttaccggtttcagtgtcatcaaatttgtttaataaaggatgaccacttatgcca

SEQ ID NO: 801 - DNA, 120 bp, human papillomavirus
  625339_25353839_333049_HPV33REF.1_1_101
tcctggacaaccgggtgctgataatagggaatgtttatccatggattataaacaaacacagttatgtttacttggatgtaagcctccaacaggggaacattggggtaaaggtgttgcttg

SEQ ID NO: 802 - DNA, 120 bp, human papillomavirus
  625339_25353839_333049_HPV33REF.1_1_102
tcaataatagtatttataagttctaaaggtggacaatcattggcaggtgctgcattagtacaagcaacacctttaccccaatgttcccctgttggaggcttacatccaagtaaacataac

SEQ ID NO: 803 - DNA, 120 bp, human papillomavirus
  625339_25353839_333049_HPV33REF.1_1_103
tactaatgcagcacctgccaatgattgtccacctttagaacttataaatactattattgaggatggtgatatggtggacacaggatttggttgcatggattttaaaacattgcaggctaa

SEQ ID NO: 804 - DNA, 120 bp, human papillomavirus
  625339_25353839_333049_HPV33REF.1_1_104
tttaaataatctggatatttgcatgtactgccacaaatatcaataggaacatcacttttattagcctgcaatgttttaaaatccatgcaaccaaatcctgtgtccaccatatcaccatcc

SEQ ID NO: 805 - DNA, 120 bp, human papillomavirus
  625339_25353839_333049_HPV33REF.1_1_105
taaaagtgatgttcctattgatatttgtggcagtacatgcaaatatccagattatttaaaaatgactagtgagccttatggtgatagtttatttttctttcttcgacgtgaacaaatgtt

SEQ ID NO: 806 - DNA, 120 bp, human papillomavirus
  625339_25353839_333049_HPV33REF.1_1_106
tacaggtcatcgggaacagcctctcctaatgtaccagccctattaaaaaagtgtcttacaaacatttgttcacgtcgaagaaagaaaaataaactatcaccataaggctcactagtcatt

SEQ ID NO: 807 - DNA, 120 bp, human papillomavirus
  625339_25353839_333049_HPV33REF.1_1_107
tgtaagacacttttttaatagggctggtacattaggagaggctgttcccgatgacctgtacattaaaggttcaggaactactgcctctattcaaagcagtgctttttttcccactcctag

SEQ ID NO: 808 - DNA, 120 bp, human papillomavirus
  625339_25353839_333049_HPV33REF.1_1_108
gcacgttgtagccaatatggcttattaaataactgagattcggaagtaaccattgatccactaggagtgggaaaaaaagcactgctttgaatagaggcagtagttcctgaacctttaatg

SEQ ID NO: 809 - DNA, 120 bp, human papillomavirus
  625339_25353839_333049_HPV33REF.1_1_109
tggatcaatggttacttccgaatctcagttatttaataagccatattggctacaacgtgcacaaggtcataataatggtatttgttggggcaatcaggtatttgttactgtggtagatac

SEQ ID NO: 810 - DNA, 120 bp, human papillomavirus
  625339_25353839_333049_HPV33REF.1_1_110
tttttatatgtactgtcactagttacttgtgtgcataaagtcatattagtactgcgagtggtatctaccacagtaacaaatacctgattgccccaacaaataccattattatgaccttgt

SEQ ID NO: 811 - DNA, 120 bp, human papillomavirus
  625339_25353839_333049_HPV33REF.1_1_111
cactcgcagtactaatatgactttatgcacacaagtaactagtgacagtacatataaaaatgaaaattttaaagaatatataagacatgttgaagaatatgatctacagtttgtttttca

SEQ ID NO: 812 - DNA, 120 bp, human papillomavirus
  625339_25353839_333049_HPV33REF.1_1_112
tctggattcatagcatgaatatatgtcataacttctgcagttaaggtaactttgcatagttgaaaaacaaactgtagatcatattcttcaacatgtcttatatattctttaaaattttca

SEQ ID NO: 813 - DNA, 120 bp, human papillomavirus
  625339_25353839_333049_HPV33REF.1_1_113
actatgcaaagttaccttaactgcagaagttatgacatatattcatgctatgaatccagatattttagaagattggcaatttggtttaacacctcctccatctgctagtttacaggatac

SEQ ID NO: 814 - DNA, 120 bp, human papillomavirus
  625339_25353839_333049_HPV33REF.1_1_114
ttttcctttggaggtactgttttttgacacgtaatagcctgagaggtaacaaacctataggtatcctgtaaactagcagatggaggaggtgttaaaccaaattgccaatcttctaaaata

SEQ ID NO: 815 - DNA, 120 bp, human papillomavirus; identical to SEQ ID NO: 173
  625339_25353839_333049_HPV33REF.1_1_115

SEQ ID NO: 816 - DNA, 120 bp, human papillomavirus
  625339_25353839_333049_HPV33REF.1_1_116
tttgctttaagacctgcctgtaataaaaacttgcgtcccaaaggaaactgatctaaatctgctgaaaatttttcctttaaatccacttcccaaaatgtatatttacctaaggggtcttcc

SEQ ID NO: 817 - DNA, 120 bp, human papillomavirus
  625339_25353839_333049_HPV33REF.1_1_117
agatttagatcagtttcctttgggacgcaagtttttattacaggcaggtcttaaagcaaaacctaaacttaaacgtgcagcccccacatccacccgcacatcgtctgcaaaacgcaaaaa

SEQ ID NO: 818 - DNA, 120 bp, human papillomavirus
  625339_25353839_333049_HPV33REF.1_1_118
gtacatagacagaacaaaacaacaacataacacaattacacaaagtgttattttttaacctttttgcgttttgcagacgatgtgcgggtggatgtgggggctgcacgtttaagtttaggt

SEQ ID NO: 819 - DNA, 120 bp, human papillomavirus
  625339_25353839_333049_HPV33REF.1_1_119
ggttaaaaaataacactttgtgtaattgtgttatgttgttgttttgttctgtctatgtactttgtgttgttgtgttgtgttgttgtttgttttttgtgtatgtgttacaatgtatgttat

SEQ ID NO: 820 - DNA, 120 bp, human papillomavirus
  625339_25353839_333049_HPV33REF.1_1_120
caagtacatagaacatgcacacaaacaagtacacataaaacaaacacagtaacatacaacataacatacattgtaacacatacacaaaaaacaaacaacaacacaacacaacaacacaaa

SEQ ID NO: 821 - DNA, 120 bp, human papillomavirus
  625339_25353839_333049_HPV33REF.1_1_121
gttgtatgttactgtgtttgttttatgtgtacttgtttgtgtgcatgttctatgtacttgtcagtttcctgtttgtgtatatgttaataaaacattgtgtgtatttgttaaactatttgt

SEQ ID NO: 822 - DNA, 120 bp, human papillomavirus
  625339_25353839_333049_HPV33REF.1_1_122
ggtagggcaagcaatacaactccttactcatataggtacacccatatacataacatacatacaaatagtttaacaaatacacacaatgttttattaacatatacacaaacaggaaactga

SEQ ID NO: 823 - DNA, 120 bp, human papillomavirus
  625339_25353839_333049_HPV33REF.1_1_123
atgtatgttatgtatatgggtgtacctatatgagtaaggagttgtattgcttgccctaccctgcattgcaatgtacctacctttatttccctatatttgtagtacctacatgtttagtat

SEQ ID NO: 824 - DNA, 120 bp, human papillomavirus
  625339_25353839_333049_HPV33REF.1_1_124
aggcatacaaaatggaggaaattgtacaatatggacactagtatgtcaaaaggtaaagcaatactaaacatgtaggtactacaaatatagggaaataaaggtaggtacattgcaatgcag

SEQ ID NO: 825 - DNA, 120 bp, human papillomavirus
  625339_25353839_333049_HPV33REF.1_1_125
tgctttaccttttgacatactagtgtccatattgtacaatttcctccattttgtatgcctaaccgttttcggttacttggcatacataccctatgacattggcagaacagttaatccttt

SEQ ID NO: 826 - DNA, 120 bp, human papillomavirus
  625339_25353839_333049_HPV33REF.1_1_126
caatgtcatagggtatgtatgccaatgcagcaagtacagacaaacacagtgcaggaaagaaaaggattaactgttctgccaatgtcatagggtatgtatgccaagtaaccgaaaacggtt

SEQ ID NO: 827 - DNA, 120 bp, human papillomavirus
  625339_25353839_333049_HPV33REF.1_1_127
tctttcctgcactgtgtttgtctgtacttgctgcattggcatacataccctatgacattggcagaacagttaatccttttctttcctgcactgtgtttgtctgtacttgctgcattgact

SEQ ID NO: 828 - DNA, 120 bp, human papillomavirus
  625339_25353839_333049_HPV33REF.1_1_128
aagcatgtgtaaactattagtacaattaagtattttgcaattgcactgcatgtatatatgagtcaatgcagcaagtacagacaaacacagtgcaggaaagaaaaggattaactgttctgc

SEQ ID NO: 829 - DNA, 120 bp, human papillomavirus
  625339_25353839_333049_HPV33REF.1_1_129
catatatacatgcagtgcaattgcaaaatacttaattgtactaatagtttacacatgcttttaggcacatatttttactttactttcaaaccttaagtgcagttttggcttacacaattg

SEQ ID NO: 830 - DNA, 120 bp, human papillomavirus
  625339_25353839_333049_HPV33REF.1_1_130
cacacctggtaataaacaggtagtgactcacttttacaaggcatagtttggcatacaaagcaattgtgtaagccaaaactgcacttaaggtttgaaagtaaagtaaaaatatgtgcctaa

SEQ ID NO: 831 - DNA, 109 bp, human papillomavirus
  625339_25353839_333049_HPV33REF.1_1_131
ctttgtatgccaaactatgccttgtaaaagtgagtcactacctgtttattaccaggtgtggactaaccgttttaggtcatattggtcatttataatcttttatataata

SEQ ID NO: 832 - DNA, 120 bp, human papillomavirus
  625339_25353836_396997_HPV35REF.1_1_1
ccctataaaaaaaacagggagtgaccgaaaacggtcgtaccgaaaacggttgccataaaagcagaagtgcacaaaaaagcagaagtggacagacattgtaaggtgcggtatgtttcagga

SEQ ID NO: 833 - DNA, 120 bp, human papillomavirus
  625339_25353836_396997_HPV35REF.1_1_2
tggatgctttcttctacctcgttgcacaaatcatgcagtttgtaaggtcgttcagctgggtcctgaaacataccgcaccttacaatgtctgtccacttctgcttttttgtgcacttctgc

SEQ ID NO: 834 - DNA, 120 bp, human papillomavirus
  625339_25353836_396997_HPV35REF.1_1_3
cccagctgaacgaccttacaaactgcatgatttgtgcaacgaggtagaagaaagcatccatgaaatttgtttgaattgtgtatactgcaaacaagaattacagcggagtgaggtatatga

SEQ ID NO: 835 - DNA, 120 bp, human papillomavirus
  625339_25353836_396997_HPV35REF.1_1_4
atgcatactccatatggctggccttctctatatactatacacaaatcatagcatgcaaagtcatatacctcactccgctgtaattcttgtttgcagtatacacaattcaaacaaatttca

SEQ ID NO: 836 - DNA, 120 bp, human papillomavirus
  625339_25353836_396997_HPV35REF.1_1_5
ctttgcatgctatgatttgtgtatagtatatagagaaggccagccatatggagtatgcatgaaatgtttaaaattttattcaaaaataagtgaatatagatggtatagatatagtgtgta

SEQ ID NO: 837 - DNA, 120 bp, human papillomavirus
  625339_25353836_396997_HPV35REF.1_1_6
atacacctaattaataaatgacataactgtttgttgcattgtttttctaacgtttctccatacacactatatctataccatctatattcacttatttttgaataaaattttaaacatttc

SEQ ID NO: 838 - DNA, 120 bp, human papillomavirus
  625339_25353836_396997_HPV35REF.1_1_7
tggagaaacgttagaaaaacaatgcaacaaacagttatgtcatttattaattaggtgtattacatgtcaaaaaccgctgtgtccagttgaaaagcaaagacatttagaagaaaaaaaacg

SEQ ID NO: 839 - DNA, 120 bp, human papillomavirus
  625339_25353836_396997_HPV35REF.1_1_8
cgtgttggtttccaacaggacatacaccgacctgtccaccgtccaccgatgttatggaatcgttttttttcttctaaatgtctttgcttttcaactggacacagcggtttttgacatgta

SEQ ID NO: 840 - DNA, 120 bp, human papillomavirus
  625339_25353836_396997_HPV35REF.1_1_9
attccataacatcggtggacggtggacaggtcggtgtatgtcctgttggaaaccaacacgtagagaaaccgaggtgtaatcatgcatggagaaataactacattgcaagactatgtttta

SEQ ID NO: 841 - DNA, 120 bp, human papillomavirus
  625339_25353836_396997_HPV35REF.1_1_10
ctctgagctgtcacacaattgctcataacagtataggtcagttgcctcgggttccaaatctaaaacatagtcttgcaatgtagttatttctccatgcatgattacacctcggtttctcta

SEQ ID NO: 842 - DNA, 120 bp, human papillomavirus
  625339_25353836_396997_HPV35REF.1_1_11
gatttggaacccgaggcaactgacctatactgttatgagcaattgtgtgacagctcagaggaggaggaagatactattgacggtccagctggacaagcaaaaccagacacctccaattat

SEQ ID NO: 843 - DNA, 120 bp, human papillomavirus
  625339_25353836_396997_HPV35REF.1_1_12
tgtgctctgtacacacagacgtagtgtcgcctcacatttacaacaggacgttacaatattataattggaggtgtctggttttgcttgtccagctggaccgtcaatagtatcttcctcctc

SEQ ID NO: 844 - DNA, 120 bp, human papillomavirus
  625339_25353836_396997_HPV35REF.1_1_13
aatattgtaacgtcctgttgtaaatgtgaggcgacactacgtctgtgtgtacagagcacacacattgacatacgtaaattggaagatttattaatgggcacatttggaatagtgtgcccc

SEQ ID NO: 845 - DNA, 120 bp, human papillomavirus
  625339_25353836_396997_HPV35REF.1_1_14
ctccccttcatctgtacctgcaggatcagccattgtagattatgctctctgtgaacagccggggcacactattccaaatgtgcccattaataaatcttccaatttacgtatgtcaatgtg

SEQ ID NO: 846 - DNA, 120 bp, human papillomavirus
  625339_25353836_396997_HPV35REF.1_1_15
ggctgttcacagagagcataatctacaatggctgatcctgcaggtacagatgaaggggaggggacgggatgtaatggatggttttttgtagaagcagtagttagtagacgtacgggggat

SEQ ID NO: 847 - DNA, 120 bp, human papillomavirus
  625339_25353836_396997_HPV35REF.1_1_16
aaagtccaccatatcctcccccctgtcacagtcatcttcattttcgtcctctgacactggatcccccgtacgtctactaactactgcttctacaaaaaaccatccattacatcccgtccc

SEQ ID NO: 848 - DNA, 120 bp, human papillomavirus
  625339_25353836_396997_HPV35REF.1_1_17
ccagtgtcagaggacgaaaatgaagatgactgtgacaggggggaggatatggtggactttataaatgatacagatatattaaacatacaggcagaaacagagacagcacaagcattattt

SEQ ID NO: 849 - DNA, 120 bp, human papillomavirus
  625339_25353836_396997_HPV35REF.1_1_18
agcatactttcgttttaggacctgtacagcctctttgtgtgtttgctcctcctgtgcatgaaataatgcttgtgctgtctctgtttctgcctgtatgtttaatatatctgtatcatttat

SEQ ID NO: 850 - DNA, 120 bp, human papillomavirus
  625339_25353836_396997_HPV35REF.1_1_19
catgcacaggaggagcaaacacacaaagaggctgtacaggtcctaaaacgaaagtatgctagtagtccacttagcagcgtgagcttatgtgttaataataacataagtccacgtttaaaa

SEQ ID NO: 851 - DNA, 120 bp, human papillomavirus
  625339_25353836_396997_HPV35REF.1_1_20
gtctggtagttcaaataatcgtcgctttgctgctgtatttttattttcaatgcaaatagcttttaaacgtggacttatgttattattaacacataagctcacgctgctaagtggactact

SEQ ID NO: 852 - DNA, 120 bp, human papillomavirus
  625339_25353836_396997_HPV35REF.1_1_21
gctatttgcattgaaaataaaaatacagcagcaaagcgacgattatttgaactaccagacagcggttatggcaattctgaagtggaaatacagcagatacaacaggtagaggggcatgat

SEQ ID NO: 853 - DNA, 120 bp, human papillomavirus
  625339_25353836_396997_HPV35REF.1_1_22
tctttcatcgctactagaggttatactatccccactgcccatactacattgttcaactgtatcatgcccctctacctgttgtatctgctgtatttccacttcagaattgccataaccgct

SEQ ID NO: 854 - DNA, 120 bp, human papillomavirus
  625339_25353836_396997_HPV35REF.1_1_23
acagttgaacaatgtagtatgggcagtggggatagtataacctctagtagcgatgaaagacatgatgagactccaacgcgagacataatacaaatactaaaatgtagtaatgcaaacgca

SEQ ID NO: 855 - DNA, 120 bp, human papillomavirus
  625339_25353836_396997_HPV35REF.1_1_24
tggtctaataagttctgtaaaactaataccaaatagttctttaaatttagccaacatagctgcgtttgcattactacattttagtatttgtattatgtctcgcgttggagtctcatcatg

SEQ ID NO: 856 - DNA, 120 bp, human papillomavirus
  625339_25353836_396997_HPV35REF.1_1_25
gctatgttggctaaatttaaagaactatttggtattagttttacagaacttattagaccatttaagagtgataaatccacatgtacagattggtgtgtggccgcatttggaatagcccca

SEQ ID NO: 857 - DNA, 120 bp, human papillomavirus
  625339_25353836_396997_HPV35REF.1_1_26
ttgtatatgtatatataaacaatatggtttaattaatgtttttaaactttccgccacacttggggctattccaaatgcggccacacaccaatctgtacatgtggatttatcactcttaaa

SEQ ID NO: 858 - DNA, 120 bp, human papillomavirus
  625339_25353836_396997_HPV35REF.1_1_27
agtgtggcggaaagtttaaaaacattaattaaaccatattgtttatatatacatatacaatgtttatcgtgttcatggggtatggtaattctagcattattacgatttaaatgtgcaaaa

SEQ ID NO: 859 - DNA, 120 bp, human papillomavirus
  625339_25353836_396997_HPV35REF.1_1_28
catacttgcagctgaaatacatagcaattttgataatagtttttcaattgttgttctgttttttgcacatttaaatcgtaataatgctagaattaccataccccatgaacacgataaaca

SEQ ID NO: 860 - DNA, 120 bp, human papillomavirus
  625339_25353836_396997_HPV35REF.1_1_29
aacagaacaacaattgaaaaactattatcaaaattgctatgtatttcagctgcaagtatgctaatacaaccaccaaaattacgtagtaccccagctgcgttatattggtttaaaacagca

SEQ ID NO: 861 - DNA, 120 bp, human papillomavirus
  625339_25353836_396997_HPV35REF.1_1_30
tactgtttgtctttgaatccattctggtgtttctccatcaacctcactaatatttgacattgctgttttaaaccaatataacgcagctggggtactacgtaattttggtggttgtattag

SEQ ID NO: 862 - DNA, 120 bp, human papillomavirus
  625339_25353836_396997_HPV35REF.1_1_31
atgtcaaatattagtgaggttgatggagaaacaccagaatggattcaaagacaaacagtattacagcatagttttaatgatgcaatatttgacctatctgaaatggtacaatgggcatat

SEQ ID NO: 863 - DNA, 120 bp, human papillomavirus
  625339_25353836_396997_HPV35REF.1_1_32
agtttctgccaattgtgcatatttatatgctatatcactatcatctataaaatcattgtcatatgcccattgtaccatttcagataggtcaaatattgcatcattaaaactatgctgtaa

SEQ ID NO: 864 - DNA, 120 bp, human papillomavirus
  625339_25353836_396997_HPV35REF.1_1_33
gacaatgattttatagatgatagtgatatagcatataaatatgcacaattggcagaaactaatagtaatgcatgtgcttttttaaaaagtaattcgcaagctaaaattgtaaaagattgt

SEQ ID NO: 865 - DNA, 120 bp, human papillomavirus
  625339_25353836_396997_HPV35REF.1_1_34
ccactgtgacattgtcatttctcttttttcagctcgtttataatgtctacacattgttgcacaatcttttacaattttagcttgcgaattactttttaaaaaagcacatgcattactatt

SEQ ID NO: 866 - DNA, 120 bp, human papillomavirus
  625339_25353836_396997_HPV35REF.1_1_35
gcaacaatgtgtagacattataaacgagctgaaaaaagagaaatgacaatgtcacagtggattaaaaggcgatgtgaaaaggtggacgatgacggtgactggagggacatagtacgattt

SEQ ID NO: 867 - DNA, 120 bp, human papillomavirus
  625339_25353836_396997_HPV35REF.1_1_36
atgtaaaaaattttttagtgcagataaaaatgccacaaaatctacttgttgatatcttaaaaatcgtactatgtccctccagtcaccgtcatcgtccaccttttcacatcgccttttaat

SEQ ID NO: 868 - DNA, 120 bp, human papillomavirus
  625339_25353836_396997_HPV35REF.1_1_37
ttaagatatcaacaagtagattttgtggcatttttatctgcactaaaaaattttttacatggtgtgcctaaaaaaaattgcatacttatatatggagcaccaaacacaggtaaatcatta

SEQ ID NO: 869 - DNA, 120 bp, human papillomavirus
  625339_25353836_396997_HPV35REF.1_1_38
tttagaatttacataggatataatagctccttgtaagaaatgcattagactcattccaaataatgatttacctgtgtttggtgctccatatataagtatgcaattttttttaggcacacc

SEQ ID NO: 870 - DNA, 120 bp, human papillomavirus
  625339_25353836_396997_HPV35REF.1_1_39
tttggaatgagtctaatgcatttcttacaaggagctattatatcctatgtaaattctaaaagccatttttggttgcagccattatatgatgccaaaatagctatgttagatgatgctaca

SEQ ID NO: 871 - DNA, 120 bp, human papillomavirus
  625339_25353836_396997_HPV35REF.1_1_40
aataggatttccatctagtgcatttcttaaatattggtctatatatgcccaacatggcgatgtagcatcatctaacatagctattttggcatcatataatggctgcaaccaaaaatggct

SEQ ID NO: 872 - DNA, 120 bp, human papillomavirus
  625339_25353836_396997_HPV35REF.1_1_41
tcgccatgttgggcatatatagaccaatatttaagaaatgcactagatggaaatcctatttcattagatgtaaagcataaagcattagtgcaattaaaatgcccacctttacttattaca

SEQ ID NO: 873 - DNA, 120 bp, human papillomavirus
  625339_25353836_396997_HPV35REF.1_1_42
gaccactaccctgctatgtaagtatggccacctgtcatctttgcctgcatttatatttgatgtaataagtaaaggtgggcattttaattgcactaatgctttatgctttacatctaatga

SEQ ID NO: 874 - DNA, 120 bp, human papillomavirus
  625339_25353836_396997_HPV35REF.1_1_43
tcaaatataaatgcaggcaaagatgacaggtggccatacttacatagcagggtagtggtctttacatttcacaatgaattcccatttgataaaaatggaaacccagtgtatgggcttaat

SEQ ID NO: 875 - DNA, 120 bp, human papillomavirus
  625339_25353836_396997_HPV35REF.1_1_44
ttcctcgtgcaaatttaatctgcaccacgtccttgagaaaaaggatttccagtttttatcattaagcccatacactgggtttccatttttatcaaatgggaattcattgtgaaatgtaaa

SEQ ID NO: 876 - DNA, 120 bp, human papillomavirus
  625339_25353836_396997_HPV35REF.1_1_45
gataaaaactggaaatcctttttctcaaggacgtggtgcagattaaatttgcacgaggaagaggacaaagaaaatgatggagacgctttcccagcgtttaagtgtgtgtcaggacaaaat

SEQ ID NO: 877 - DNA, 120 bp, human papillomavirus
  625339_25353836_396997_HPV35REF.1_1_46
agtttccaatactgtatgtgatcagacaaacatgtgctatcagtctcgtaatgttctagtattttgtcctgacacacacttaaacgctgggaaagcgtctccatcattttctttgtcctc

SEQ ID NO: 878 - DNA, 120 bp, human papillomavirus
  625339_25353836_396997_HPV35REF.1_1_47
actagaacattacgagactgatagcacatgtttgtctgatcacatacagtattggaaactgattcgtcttgaatgtgcagtattttataaagcaagagaaatgggaattaaaactcttaa

SEQ ID NO: 879 - DNA, 120 bp, human papillomavirus
  625339_25353836_396997_HPV35REF.1_1_48
agttcaattgcttgcattgctttggcttttgaaatggcctgcgttggaaccacttggtggttaagagttttaattcccatttctcttgctttataaaatactgcacattcaagacgaatc

SEQ ID NO: 880 - DNA, 120 bp, human papillomavirus
  625339_25353836_396997_HPV35REF.1_1_49
ccaccaagtggttccaacgcaggccatttcaaaagccaaagcaatgcaagcaattgaactgcaattaatgttagagacattaaatacaactgagtatagcacagaaacatggacactgca

SEQ ID NO: 881 - DNA, 120 bp, human papillomavirus
  625339_25353836_396997_HPV35REF.1_1_50
accccatgttttttaaaacatccttgtggaactgttgtatataattcaatacttgtttcttgcagtgtccatgtttctgtgctatactcagttgtatttaatgtctctaacattaattgc

SEQ ID NO: 882 - DNA, 120 bp, human papillomavirus
  625339_25353836_396997_HPV35REF.1_1_51
agaaacaagtattgaattatatacaacagttccacaaggatgttttaaaaaacatggggttacagtggaagtacaatttgatggtgataaacaaaatactatgcattatactaattggac

SEQ ID NO: 883 - DNA, 120 bp, human papillomavirus
  625339_25353836_396997_HPV35REF.1_1_52
ttataatttaccagtccctttacaacagtacatatactgtcctctaatatatatatatgtgtccaattagtataatgcatagtattttgtttatcaccatcaaattgtacttccactgta

SEQ ID NO: 884 - DNA, 120 bp, human papillomavirus
  625339_25353836_396997_HPV35REF.1_1_53
acatatatatatattagaggacagtatatgtactgttgtaaagggactggtaaattataaaggtatttattatgtgcatcagggtgtagaaacatattatgttacttttagggaagaggc

SEQ ID NO: 885 - DNA, 120 bp, human papillomavirus
  625339_25353836_396997_HPV35REF.1_1_54
caaacaattacctgaccacccacatgcacttcccatatatttttttttccatactttttagcctcttccctaaaagtaacataatatgtttctacaccctgatgcacataataaatacct

SEQ ID NO: 886 - DNA, 120 bp, human papillomavirus
  625339_25353836_396997_HPV35REF.1_1_55
taaaaagtatggaaaaaaaaatatatgggaagtgcatgtgggtggtcaggtaattgtttgtcctgaatctgtatttagcagcacagaactatccactgctgaaattgctacacagctaca

SEQ ID NO: 887 - DNA, 120 bp, human papillomavirus
  625339_25353836_396997_HPV35REF.1_1_56
tgggtttctgtggtgcccacggagcaggctttggtatgggtctcggtggtgttgtaggcgtgtagctgtgtagcaatttcagcagtggatagttctgtgctgctaaatacagattcagga

SEQ ID NO: 888 - DNA, 120 bp, human papillomavirus
  625339_25353836_396997_HPV35REF.1_1_57
cgcctacaacaccaccgagacccataccaaagcctgctccgtgggcaccacagaaacccagaagacaaatcacaaacgacttcgagggggtaccgagctcccctacaaccccaccaagcg

SEQ ID NO: 889 - DNA, 120 bp, human papillomavirus
  625339_25353836_396997_HPV35REF.1_1_58
gtgcagtcagatgtagagtagacccctctgtcaacactgtccacggcactgagtcgcactcgcttggtggggttgtaggggagctcggtaccccctcgaagtcgtttgtgatttgtcttc

SEQ ID NO: 890 - DNA, 120 bp, human papillomavirus
  625339_25353836_396997_HPV35REF.1_1_59
agtgcgactcagtgccgtggacagtgttgacagaggggtctactctacatctgactgcacaaacaaagaccggtgtggtagttgtagtacaactacacctatagtacatttaaaaggtga

SEQ ID NO: 891 - DNA, 120 bp, human papillomavirus
  625339_25353836_396997_HPV35REF.1_1_60
tcttgatacaatgctttatatttacccaatctatatcttaaacactttaatgtatttgcatcaccttttaaatgtactataggtgtagttgtactacaactaccacaccggtctttgttt

SEQ ID NO: 892 - DNA, 120 bp, human papillomavirus
  625339_25353836_396997_HPV35REF.1_1_61
tgcaaatacattaaagtgtttaagatatagattgggtaaatataaagcattgtatcaagatgcttcatctacatggagatggacatgtacaaacgataaaaaacaaatagcaattgtaac

SEQ ID NO: 893 - DNA, 120 bp, human papillomavirus
  625339_25353836_396997_HPV35REF.1_1_62
ttaggtatttttactgtagttaaaaatttatccctttgatattctgttgtgtaagttaatgttacaattgctatttgttttttatcgtttgtacatgtccatctccatgtagatgaagca

SEQ ID NO: 894 - DNA, 120 bp, human papillomavirus
  625339_25353836_396997_HPV35REF.1_1_63
attaacttacacaacagaatatcaaagggataaatttttaactacagtaaaaatacctaacacagttacagtgtctaaaggatatatgtctatatgatagaccttacagcttccagtact

SEQ ID NO: 895 - DNA, 120 bp, human papillomavirus
  625339_25353836_396997_HPV35REF.1_1_64
acgtacaagcagacacaagcacaaaagcacacaaaagcacaacaaaaagcacagcaacacagtactggaagctgtaaggtctatcatatagacatatatcctttagacactgtaactgtg

SEQ ID NO: 896 - DNA, 120 bp, human papillomavirus
  625339_25353836_396997_HPV35REF.1_1_65
gtgttgctgtgctttttgttgtgcttttgtgtgcttttgtgcttgtgtctgcttgtacgttcgctattgctatctgtgtcattatactcagcattaatattactggttttaatactgtgg

SEQ ID NO: 897 - DNA, 120 bp, human papillomavirus
  625339_25353836_396997_HPV35REF.1_1_66
aggtatatacaaaaagcaaagaaaacaacaaaagcaacgtagtggtgttgctacagtaacccacagtattaaaaccagtaatattaatgctgagtataatgacacagatagcaatagcga

SEQ ID NO: 898 - DNA, 120 bp, human papillomavirus
  625339_25353836_396997_HPV35REF.1_1_67
gttactgtagcaacaccactacgttgcttttgttgttttctttgctttttgtatatacctatgggaatgattaacgctcatgcacaatatttggcagtacagtaattgtatacaaacatt

SEQ ID NO: 899 - DNA, 120 bp, human papillomavirus
  625339_25353836_396997_HPV35REF.1_1_68
tatacaatgaacaacaaaaaataaaaccaccatacacacatgttacacagtaccaaacacaatgtttgtatacaattactgtactgccaaatattgtgcatgagcgttaatcattcccat

SEQ ID NO: 900 - DNA, 120 bp, human papillomavirus
  625339_25353836_396997_HPV35REF.1_1_69
gtgtttggtactgtgtaacatgtgtgtatggtggttttattttttgttgttcattgtatattttgtttttttactgtttttaaacatttttatttctgtgtttttaataaattgatcaca

SEQ ID NO: 901 - DNA, 120 bp, human papillomavirus
  625339_25353836_396997_HPV35REF.1_1_70
tgtgttgcagatgcacgtttaacacgttttgtagaccttttgtgtcgcatggttataccatgtgatcaatttattaaaaacacagaaataaaaatgtttaaaaacagtaaaaaaacaaaa

SEQ ID NO: 902 - DNA, 120 bp, human papillomavirus
  625339_25353836_396997_HPV35REF.1_1_71
tggtataaccatgcgacacaaaaggtctacaaaacgtgttaaacgtgcatctgcaacacaactatatcgtacttgcaaagctgcaggaacttgtccaccagatgttatacctaaggttga

SEQ ID NO: 903 - DNA, 120 bp, human papillomavirus
  625339_25353836_396997_HPV35REF.1_1_72
cccccaaaaaacacagccatgctgccatattttaaaatttgatcagcaacagtattaccctcaaccttaggtataacatctggtggacaagttcctgcagctttgcaagtacgatatagt

SEQ ID NO: 904 - DNA, 120 bp, human papillomavirus
  625339_25353836_396997_HPV35REF.1_1_73
gggtaatactgttgctgatcaaattttaaaatatggcagcatggctgtgttttttggggggttaggaattggttctggatctggcacaggtggaagatctggatatgttccactgggtac

SEQ ID NO: 905 - DNA, 120 bp, human papillomavirus
  625339_25353836_396997_HPV35REF.1_1_74
atactttccacagttacagggggtcgtataggaatgtttgtggcagccgttggaggtgttgtacccagtggaacatatccagatcttccacctgtgccagatccagaaccaattcctaac

SEQ ID NO: 906 - DNA, 120 bp, human papillomavirus
  625339_25353836_396997_HPV35REF.1_1_75
aacacctccaacggctgccacaaacattcctatacgaccccctgtaactgtggaaagtataccattagacacaattggccctttagattcttctatagtgtcattagtagaggaaactag

SEQ ID NO: 907 - DNA, 120 bp, human papillomavirus
  625339_25353836_396997_HPV35REF.1_1_76
gtaaaacctgttgtaggtgggacccttggtgtaacaacaggggcaccagactcaataaaactagtttcctctactaatgacactatagaagaatctaaagggccaattgtgtctaatggt

SEQ ID NO: 908 - DNA, 120 bp, human papillomavirus
  625339_25353836_396997_HPV35REF.1_1_77
ttttattgagtctggtgcccctgttgttacaccaagggtcccacctacaacaggttttacaataaccacatctacagataccacacctgctattttagatgtgacatccataagtacaca

SEQ ID NO: 909 - DNA, 120 bp, human papillomavirus
  625339_25353836_396997_HPV35REF.1_1_78
gaagtttctgcaggcgtgggtgggtgtaaaacagaaggatcagtgaaagtaggattatcatgtgtacttatggatgtcacatctaaaatagcaggtgtggtatctgtagatgtggttatt

SEQ ID NO: 910 - DNA, 120 bp, human papillomavirus
  625339_25353836_396997_HPV35REF.1_1_79
tgataatcctactttcactgatccttctgttttacacccacccacgcctgcagaaacttcaggtcattttgtactttcatcatcttctattagtacacataattatgaagaaatccctat

SEQ ID NO: 911 - DNA, 120 bp, human papillomavirus
  625339_25353836_396997_HPV35REF.1_1_80
cctggaataggcgtgctattagttatattattgctgtctgtggaaacaataaaagtatccatagggatttcttcataattatgtgtactaatagaagatgatgaaagtacaaaatgacct

SEQ ID NO: 912 - DNA, 120 bp, human papillomavirus
  625339_25353836_396997_HPV35REF.1_1_81
ggatacttttattgtttccacagacagcaataatataactaatagcacgcctattccagggtctcgccctacgacacgcctaggattatatagtaaaggtacccagcaggttaaggttgt

SEQ ID NO: 913 - DNA, 120 bp, human papillomavirus
  625339_25353836_396997_HPV35REF.1_1_82
tcatatgcaggattatcatatgtaataagttttgcaggagaagtcataaaggcagggtcaacaaccttaacctgctgggtacctttactatataatcctaggcgtgtcgtagggcgagac

SEQ ID NO: 914 - DNA, 120 bp, human papillomavirus
  625339_25353836_396997_HPV35REF.1_1_83
tgaccctgcctttatgacttctcctgcaaaacttattacatatgataatcctgcatatgaaggccttaaccctgatacaaccttacaatttgagcatgaggatattagcttagctccgga

SEQ ID NO: 915 - DNA, 120 bp, human papillomavirus
  625339_25353836_396997_HPV35REF.1_1_84
gtgcctttcctagatgttagtgcaggcctatgtaaagctataatgtccataaagtcaggatccggagctaagctaatatcctcatgctcaaattgtaaggttgtatcagggttaaggcct

SEQ ID NO: 916 - DNA, 120 bp, human papillomavirus
  625339_25353836_396997_HPV35REF.1_1_85
tcctgactttatggacattatagctttacataggcctgcactaacatctaggaaaggcactattagatatagtagagtaggtaataaacgtactatgcatacacgaagtggaaaagctat

SEQ ID NO: 917 - DNA, 120 bp, human papillomavirus
  625339_25353836_396997_HPV35REF.1_1_86
tgtaattctatatcttcagtaatactacttaaatcctgataataatgtacccgtgcccctatagcttttccacttcgtgtatgcatagtacgtttattacctactctactatatctaata

SEQ ID NO: 918 - DNA, 120 bp, human papillomavirus
  625339_25353836_396997_HPV35REF.1_1_87
aggggcacgggtacattattatcaggatttaagtagtattactgaagatatagaattacaacccttacaacatgtaccatcctctttaccacataccactgtttcaacatcattaaatga

SEQ ID NO: 919 - DNA, 120 bp, human papillomavirus
  625339_25353836_396997_HPV35REF.1_1_88
gatgctgaaaatataatatcttcctcagtatctataggagcataaatatcaaacataccatcatttaatgatgttgaaacagtggtatgtggtaaagaggatggtacatgttgtaagggt

SEQ ID NO: 920 - DNA, 120 bp, human papillomavirus
  625339_25353836_396997_HPV35REF.1_1_89
tggtatgtttgatatttatgctcctatagatactgaggaagatattatattttcagcatcttctaacaatactttatatactacatctaacactgcatatgttcctagcaatactactat

SEQ ID NO: 921 - DNA, 120 bp, human papillomavirus
  625339_25353836_396997_HPV35REF.1_1_90
gagttaaatacaatgtctggccctgctgttataggaatatcatagccactacttaatggtatagtagtattgctaggaacatatgcagtgttagatgtagtatataaagtattgttagaa

SEQ ID NO: 922 - DNA, 120 bp, human papillomavirus
  625339_25353836_396997_HPV35REF.1_1_91
accattaagtagtggctatgatattcctataacagcagggccagacattgtatttaactctaatactattactaacactgtactaccggtacccacaggtcctatatattctattattgc

SEQ ID NO: 923 - DNA, 120 bp, human papillomavirus
  625339_25353836_396997_HPV35REF.1_1_92
cgtttacgacgtcgttttaataaataataactagggtgtaaataaaagtcacccccatctgcaataatagaatatataggacctgtgggtaccggtagtacagtgttagtaatagtatta

SEQ ID NO: 924 - DNA, 120 bp, human papillomavirus
  625339_25353836_396997_HPV35REF.1_1_93
agatgggggtgacttttatttacaccctagttattatttattaaaacgacgtcgtaaacgtatcccatatttttttgcagatgtctctgtggcggtctaacgaagccactgtctacctgc

SEQ ID NO: 925 - DNA, 120 bp, human papillomavirus
  625339_25353836_396997_HPV35REF.1_1_94
agatgtttgtgcgtgttacatattcatcagtgctaacaaccttagacactgacactggaggcaggtagacagtggcttcgttagaccgccacagagacatctgcaaaaaaatatgggata

SEQ ID NO: 926 - DNA, 120 bp, human papillomavirus
  625339_25353836_396997_HPV35REF.1_1_95
ctccagtgtcagtgtctaaggttgttagcactgatgaatatgtaacacgcacaaacatctactatcatgcaggcagttctaggctattagctgtgggtcacccatactatgctattaaaa

SEQ ID NO: 927 - DNA, 120 bp, human papillomavirus
  625339_25353836_396997_HPV35REF.1_1_96
taaatactctgtattgcaaaccagataccttgggtactgctattttattagaatcttgttttttaatagcatagtatgggtgacccacagctaatagcctagaactgcctgcatgatagt

SEQ ID NO: 928 - DNA, 120 bp, human papillomavirus
  625339_25353836_396997_HPV35REF.1_1_97
aacaagattctaataaaatagcagtacccaaggtatctggtttgcaatacagagtatttagagtaaaattaccagatcctaataagtttggatttccagacacatcattttatgatcctg

SEQ ID NO: 929 - DNA, 120 bp, human papillomavirus
  625339_25353836_396997_HPV35REF.1_1_98
ccaatggctgaccacgacctacttcaactcctgtacaggcccaaaccaaacgctgggaggcaggatcataaaatgatgtgtctggaaatccaaacttattaggatctggtaattttactc

SEQ ID NO: 930 - DNA, 120 bp, human papillomavirus
  625339_25353836_396997_HPV35REF.1_1_99
cctcccagcgtttggtttgggcctgtacaggagttgaagtaggtcgtggtcagccattgggtgtaggtattagtggtcatcctttattaaataaattggatgatactgaaaattctaata

SEQ ID NO: 931 - DNA, 120 bp, human papillomavirus
  625339_25353836_396997_HPV35REF.1_1_100
tttgtttataatccatagaaatgcattccctgttatctgtaccagagttaccaacatatttattagaattttcagtatcatccaatttatttaataaaggatgaccactaatacctacac

SEQ ID NO: 932 - DNA, 120 bp, human papillomavirus
  625339_25353836_396997_HPV35REF.1_1_101
aatatgttggtaactctggtacagataacagggaatgcatttctatggattataaacaaacacaattgtgtttaataggttgtaggcctcctataggtgaacattggggaaaaggcacac

SEQ ID NO: 933 - DNA, 120 bp, human papillomavirus
  625339_25353836_396997_HPV35REF.1_1_102
cagtgtttagtaactccaaaggaggacattctcctgcttttacctggttagcattacaaggtgtgccttttccccaatgttcacctataggaggcctacaacctattaaacacaattgtg

SEQ ID NO: 934 - DNA, 120 bp, human papillomavirus
  625339_25353836_396997_HPV35REF.1_1_103
cttgtaatgctaaccaggtaaaagcaggagaatgtcctcctttggagttactaaacactgtactacaagacggggacatggtagacacaggatttggtgcaatggattttactacattac

SEQ ID NO: 935 - DNA, 120 bp, human papillomavirus
  625339_25353836_396997_HPV35REF.1_1_104
aatcaggatatttgcaaatggaactgcatatatctaggggaacatcacttttattagcttgtaatgtagtaaaatccattgcaccaaatcctgtgtctaccatgtccccgtcttgtagta

SEQ ID NO: 936 - DNA, 120 bp, human papillomavirus
  625339_25353836_396997_HPV35REF.1_1_105
aagctaataaaagtgatgttcccctagatatatgcagttccatttgcaaatatcctgattatctaaaaatggtttctgagccatatggagatatgttatttttttatttacgtagggagc

SEQ ID NO: 937 - DNA, 120 bp, human papillomavirus
  625339_25353836_396997_HPV35REF.1_1_106
ctgcaggtactgtttcacctacagttccagccctattaaataaatgtctaacaaacatttgctccctacgtaaataaaaaaataacatatctccatatggctcagaaaccatttttagat

SEQ ID NO: 938 - DNA, 120 bp, human papillomavirus
  625339_25353836_396997_HPV35REF.1_1_107
aaatgtttgttagacatttatttaatagggctggaactgtaggtgaaacagtacctgcagacctatatattaagggtaccactggcacattgcctagtactagttattttcctactccta

SEQ ID NO: 939 - DNA, 120 bp, human papillomavirus
  625339_25353836_396997_HPV35REF.1_1_108
cacgttgcaaccaatatggtttattaaatatttgtgcatcggaggttaccatagagccactaggagtaggaaaataactagtactaggcaatgtgccagtggtacccttaatatataggt

SEQ ID NO: 940 - DNA, 120 bp, human papillomavirus
  625339_25353836_396997_HPV35REF.1_1_109
gtggctctatggtaacctccgatgcacaaatatttaataaaccatattggttgcaacgtgcacaaggccataataatggtatttgttggagtaaccaattgtttgttactgtagttgata

SEQ ID NO: 941 - DNA, 120 bp, human papillomavirus
  625339_25353836_396997_HPV35REF.1_1_110
tatatgtactgtcactagaagacacagcagaacacacagacatatttgtactacgggttgtatcaactacagtaacaaacaattggttactccaacaaataccattattatggccttgtg

SEQ ID NO: 942 - DNA, 120 bp, human papillomavirus
  625339_25353836_396997_HPV35REF.1_1_111
caacccgtagtacaaatatgtctgtgtgttctgctgtgtcttctagtgacagtacatataaaaatgacaattttaaggaatatttaaggcatggtgaagaatatgatttacagtttattt

SEQ ID NO: 943 - DNA, 120 bp, human papillomavirus
  625339_25353836_396997_HPV35REF.1_1_112
ggttcatactatgaatatatgtcataacatctgctgttagtgttattttacataactgaaaaataaactgtaaatcatattcttcaccatgccttaaatattccttaaaattgtcatttt

SEQ ID NO: 944 - DNA, 120 bp, human papillomavirus
  625339_25353836_396997_HPV35REF.1_1_113
ttcagttatgtaaaataacactaacagcagatgttatgacatatattcatagtatgaacccgtccattttagaggattggaattttggccttacaccaccgccttctggtaccttagagg

SEQ ID NO: 945 - DNA, 120 bp, human papillomavirus
  625339_25353836_396997_HPV35REF.1_1_114
gttttggtgcactgggtttttgacaagttacagcctgtgatgttacatagcgatatgtgtcctctaaggtaccagaaggcggtggtgtaaggccaaaattccaatcctctaaaatggacg

SEQ ID NO: 946 - DNA, 120 bp, human papillomavirus
  625339_25353836_396997_HPV35REF.1_1_115
acacatatcgctatgtaacatcacaggctgtaacttgtcaaaaacccagtgcaccaaaacctaaagatgatccattaaaaaattatactttttgggaggttgatttaaaggaaaagtttt

SEQ ID NO: 947 - DNA, 120 bp, human papillomavirus
  625339_25353836_396997_HPV35REF.1_1_116
cctttagtcctgcttgtaacaaaaatttacggcccaacggaaattgatctaagtctgcagaaaacttttcctttaaatcaacctcccaaaaagtataattttttaatggatcatctttag

SEQ ID NO: 948 - DNA, 120 bp, human papillomavirus
  625339_25353836_396997_HPV35REF.1_1_117
ctgcagacttagatcaatttccgttgggccgtaaatttttgttacaagcaggactaaaggccaggcctaattttagattaggcaagcgtgcagctccagcatctacatctaaaaaatctt

SEQ ID NO: 949 - DNA, 120 bp, human papillomavirus
  625339_25353836_396997_HPV35REF.1_1_118
cacacagtatacatgcatacacatttacacattaactttttacttttctacgtttagtagaagattttttagatgtagatgctggagctgcacgcttgcctaatctaaaattaggcctgg

SEQ ID NO: 950 - DNA, 120 bp, human papillomavirus
  625339_25353836_396997_HPV35REF.1_1_119
ctactaaacgtagaaaagtaaaaagttaatgtgtaaatgtgtatgcatgtatactgtgtgttatgtgttgtagtgcttgtatatatattatgtgttgtggtgcctgtttgtgttgtacat

SEQ ID NO: 951 - DNA, 120 bp, human papillomavirus
  625339_25353836_396997_HPV35REF.1_1_120
acatacaaaaaacacccacgtacaacacattgcacaatattatacacacatttacacgccatgtacaacacaaacaggcaccacaacacataatatatatacaagcactacaacacataa

SEQ ID NO: 952 - DNA, 120 bp, human papillomavirus
  625339_25353836_396997_HPV35REF.1_1_121
ggcgtgtaaatgtgtgtataatattgtgcaatgtgttgtacgtgggtgttttttgtatgtatgttgttgtatgtatgtcagtacgcaataaaagtgatgtgtgtgtttataattaacact

SEQ ID NO: 953 - DNA, 120 bp, human papillomavirus
  625339_25353836_396997_HPV35REF.1_1_122
tatagcgtgtactgtaattatgtaagtcatatgggtgcacccatagtcatacaacaatacagtgttaattataaacacacacatcacttttattgcgtactgacatacatacaacaacat

SEQ ID NO: 954 - DNA, 120 bp, human papillomavirus
  625339_25353836_396997_HPV35REF.1_1_123
gtattgttgtatgactatgggtgcacccatatgacttacataattacagtacacgctatatgttgtatataacaattctacctccattttgtgtgttagtgtcctttacattacctttca

SEQ ID NO: 955 - DNA, 120 bp, human papillomavirus
  625339_25353836_396997_HPV35REF.1_1_124
tgaggtaggaatgtttttgtaaaaaacatataaagcttgccaacagcaaccgaaatcggttgaaaggtaatgtaaaggacactaacacacaaaatggaggtagaattgttatatacaaca

SEQ ID NO: 956 - DNA, 120 bp, human papillomavirus
  625339_25353836_396997_HPV35REF.1_1_125
accgatttcggttgctgttggcaagctttatatgttttttacaaaaacattcctacctcagcagaacacttaatccttgtgttcctgatatatattgtttgccaactttatattggcttt

SEQ ID NO: 957 - DNA, 120 bp, human papillomavirus
  625339_25353836_396997_HPV35REF.1_1_126
tggaccacaagtatgaaaaatgactaatactgcaagatgaatcaagtttaaagattggcaaaagccaatataaagttggcaaacaatatatatcaggaacacaaggattaagtgttctgc

SEQ ID NO: 958 - DNA, 120 bp, human papillomavirus
  625339_25353836_396997_HPV35REF.1_1_127
tgccaatctttaaacttgattcatcttgcagtattagtcatttttcatacttgtggtccacccacacttgtaacacttgtaacagtgcttttaggcacatattttttgcatttctaaagg

SEQ ID NO: 959 - DNA, 120 bp, human papillomavirus
  625339_25353836_396997_HPV35REF.1_1_128
atgtgtagggtggtgttggcaaacacacataatatgtaaagccaagtgtgcaattaaagccctttagaaatgcaaaaaatatgtgcctaaaagcactgttacaagtgttacaagtgtggg

SEQ ID NO: 960 - DNA, 120 bp, human papillomavirus
  625339_25353836_396997_HPV35REF.1_1_129
gctttaattgcacacttggctttacatattatgtgtgtttgccaacaccaccctacacatcctgccaactttaagttaaaacatgcatgtaaaacattactcactgtattacacattgtt

SEQ ID NO: 961 - DNA, 120 bp, human papillomavirus
  625339_25353836_396997_HPV35REF.1_1_130
ataaaaagaaatgcttataaaactgtaatccaaatcggttggacacacctgtgtgcatataacaatgtgtaatacagtgagtaatgttttacatgcatgttttaacttaaagttggcagg

SEQ ID NO: 962 - DNA, 120 bp, human
  186573_9856646_PTEN.1_1
tcagccacaggctcccagacatgacagccatcatcaaagagatcgttagcagaaacaaaaggagatatcaagaggatggattcgacttagacttgacctgtatccatttctgcggctgct

SEQ ID NO: 963 - DNA, 120 bp, human
  186573_9856647_PTEN_1
cttaactaaagtactcagatatttatccaaacattattgctatgggatttcctgcagaaagacttgaaggcgtatacaggaacaatattgatgatgtagtaaggtaagaatgctttgatt

SEQ ID NO: 964 - DNA, 120 bp, human
  186573_9856648_PTEN_1
gttaatggtggctttttgtttgtttgttttgttttaaggtttttggattcaaagcataaaaaccattacaagatatacaatctgtaagtatgttttcttatttgtatgcttgcaaatatc

SEQ ID NO: 965 - DNA, 120 bp, human
  186573_9856649_PTEN_1
aaagataactttatatcacttttaaacttttcttttagttgtgctgaaagacattatgacaccgccaaatttaattgcagaggtaggtatgaatgtactgtactatgttgtataacttaa

SEQ ID NO: 966 - DNA, 120 bp, human
  186573_9856650_PTEN.1_1
ttgcacaatatccttttgaagaccataacccaccacagctagaacttatcaaacccttttgtgaagatcttgaccaatggctaagtgaagatgacaatcatgttgcagcaattcactgta

SEQ ID NO: 967 - DNA, 120 bp, human
  186573_9856650_PTEN.1_2
aagctggaaagggacgaactggtgtaatgatatgtgcatatttattacatcggggcaaatttttaaaggcacaagaggccctagatttctatggggaagtaaggaccagagacaaaaagg

SEQ ID NO: 968 - DNA, 120 bp, human
  186573_9856651_PTEN_1
atggaacattttttttcaatttggcttctcttttttttctgtccaccagggagtaactattcccagtcagaggcgctatgtgtattattatagctacctgttaaagaatcatctggatta

SEQ ID NO: 969 - DNA, 120 bp, human
  186573_9856651_PTEN_2
tagaccagtggcactgttgtttcacaagatgatgtttgaaactattccaatgttcagtggcggaacttgcagtaagtgcttgaaattctcatccttccatgtattggaacagttttctta

SEQ ID NO: 970 - DNA, 120 bp, human
  186573_9856652_PTEN_1
ctgtgaaataatactggtatgtatttaaccatgcagatcctcagtttgtggtctgccagctaaaggtgaagatatattcctccaattcaggacccacacgacgggaagacaagttcatgt

SEQ ID NO: 971 - DNA, 120 bp, human
  186573_9856652_PTEN_2
actttgagttccctcagccgttacctgtgtgtggtgatatcaaagtagagttcttccacaaacagaacaagatgctaaaaaaggtttgtactttactttcattgggagaaatatccaaaa

SEQ ID NO: 972 - DNA, 120 bp, human
  186573_9856653_PTEN.1_1
tttttaggacaaaatgtttcacttttgggtaaatacattcttcataccaggaccagaggaaacctcagaaaaagtagaaaatggaagtctatgtgatcaagaaatcgatagcatttgcag

SEQ ID NO: 973 - DNA, 120 bp, human
  186573_9856653_PTEN.1_2
tatagagcgtgcagataatgacaaggaatatctagtacttactttaacaaaaaatgatcttgacaaagcaaataaagacaaagccaaccgatacttttctccaaattttaaggtcagtta

SEQ ID NO: 974 - DNA, 120 bp, human
  186573_9856654_PTEN_1
ttttcattttaaattttctttctctaggtgaagctgtacttcacaaaaacagtagaggagccgtcaaatccagaggctagcagttcaacttctgtaacaccagatgttagtgacaatgaa

SEQ ID NO: 975 - DNA, 120 bp, human
  186573_9856654_PTEN_2
cctgatcattatagatattctgacaccactgactctgatccagagaatgaaccttttgatgaagatcagcatacacaaattacaaaagtctgaatttttttttatcaagagggataaaac

SEQ ID NO: 976 - DNA, 120 bp, human
  186573_9856655_HRAS_1
cggggagccggggtcatccggtgggcgtggcggccgccctgggagtccccctcacctgcgtcaggagagcacacacttgcagctcatgcagccggggccactctcatcaggagggttcag

SEQ ID NO: 977 - DNA, 120 bp, human
  186573_9856655_HRAS_2
cttccgcagcttgtgctgccggatctcacgcaccaacgtgtagaaggcatcctccactccctgggaaaggagggatgggatcaggagggaccggcctgtggccgcctgcctgggtgaggg

SEQ ID NO: 978 - DNA, 120 bp, human
  186573_9856656_HRAS_1
acagcgcgaggggccgctgggtcacatgggtcccggggggtcccagagggtcccggagctggagctagagccagagcggctgccctgtgtcaagggagagggtcagtgagtgctgctccc

SEQ ID NO: 979 - DNA, 120 bp, human
  186573_9856656_HRAS_2
tggctggggcggggcggggcgggtccctggctagctgtggggtggagagctgcctcacctgccgggtcttggccgaggtctcgatgtaggggatgccgtagcttcgggcgaggtcctgag

SEQ ID NO: 980 - DNA, 120 bp, human
  186573_9856656_HRAS_3
cctgccgagattccacagtgcgtgcagccaggtcacacttgttccccaccagcaccatgggcacgtcatccgagtccttcacccgtttgatctgctccctgagaggtggaaagcgagagc

SEQ ID NO: 981 - DNA, 120 bp, human
  186573_9856657_HRAS_1
ggctcccgggccagcctcacggggttcacctgtactggtggatgtcctcaaaagacttggtgttgttgatggcaaacacacacaggaagccctccccggtgcgcatgtactggtcccgca

SEQ ID NO: 982 - DNA, 120 bp, human
  186573_9856657_HRAS_2
tggcgctgtactcctcctggccggcggtatccaggatgtccaacaggcacgtctccccatcaatgaccacctgcttccggtaggaatcctgcaggaggacagggctcagggaccccctca

SEQ ID NO: 983 - DNA, 120 bp, human
  186573_9856658_HRAS.1_1
tcacctctatagtggggtcgtattcgtccacaaaatggttctggatcagctggatggtcagcgcactcttgcccacaccgccggcgcccaccaccaccagcttatattccgtcatcgctc

SEQ ID NO: 984 - DNA, 120 bp, human
  186573_9856659_TP53_1
aacaagaagtggagaatgtcagtctgagtcaggcccttctgtcttgaacatgagttttttatggcgggaggtagactgaccctttttggacttcaggtggctgtaggagacagaagcagg

SEQ ID NO: 985 - DNA, 120 bp, human
  186573_9856660_TP53_1
actcacctggagtgagccctgctcccccctggctccttcccagcctgggcatccttgagttccaaggcctcattcagctctcggaacatctcgaagcgctcacgcccacggatctgcagc

SEQ ID NO: 986 - DNA, 120 bp, human
  186573_9856661_TP53.1_1
cgtaagtcaagtagcatctgtatcaggcaaagtcatagaaccattttcatgctctctttaacaattttctttttgaaagctggtctggtcctttaaaatatatattatggtataagttgg

SEQ ID NO: 987 - DNA, 120 bp, human
  186573_9856662_TP53_1
taagaggtcccaagacttagtacctgaagggtgaaatattctccatccagtggtttcttctttggctggggagaggagctggtgttgttgggcagtgctaggaaagaggcaaggaaaggt

SEQ ID NO: 988 - DNA, 120 bp, human
  186573_9856663_TP53_1
ggcataactgcacccttggtctcctccaccgcttcttgtcctgcttgcttacctcgcttagtgctccctgggggcagctcgtggtgaggctcccctttcttgcggagattctcttcctct

SEQ ID NO: 989 - DNA, 120 bp, human
  186573_9856663_TP53_2
gtgcgccggtctctcccaggacaggcacaaacacgcacctcaaagctgttccgtcccagtagattaccactactcaggataggaaaagagaagcaagaggcagtaaggaaatcaggtcct

SEQ ID NO: 990 - DNA, 120 bp, human
  186573_9856664_TP53.1_1
ctgacctggagtcttccagtgtgatgatggtgaggatgggcctccggttcatgccgcccatgcaggaactgttacacatgtagttgtagtggatggtggtacagtcagagccaacctagg

SEQ ID NO: 991 - DNA, 120 bp, human
  186573_9856665_TP53_1
agacctcaggcggctcatagggcaccaccacactatgtcgaaaagtgtttctgtcatccaaatactccacacgcaaatttccttccactcggataagatgctgaggaggggccagaccta

SEQ ID NO: 992 - DNA, 120 bp, human
  186573_9856666_TP53_1
ctgtcgtctctccagccccagctgctcaccatcgctatctgagcagcgctcatggtgggggcagcgcctcacaacctccgtcatgtgctgtgactgcttgtagatggccatggcgcggac

SEQ ID NO: 993 - DNA, 120 bp, human
  186573_9856666_TP53_2
gcgggtgccgggcgggggtgtggaatcaacccacagctgcacagggcaggtcttggccagttggcaaaacatcttgttgagggcaggggagtactgtaggaagaggaaggagacagagtt

SEQ ID NO: 994 - DNA, 120 bp, human
  186573_9856667_TP53_1
ccaggcattgaagtctcatggaagccagcccctcagggcaactgaccgtgcaagtcacagacttggctgtcccagaatgcaagaagcccagacggaaaccgtagctgccctggtaggttt

SEQ ID NO: 995 - DNA, 120 bp, human
  186573_9856667_TP53_2
tctgggaagggacagaagatgacaggggccaggagggggctggtgcaggggccgccggtgtaggagctgctggtgcaggggccacggggggagcagcctctggcattctgggagcttcat

SEQ ID NO: 996 - DNA, 120 bp, human
  186573_9856667_TP53_3
ctggacctgggtcttcagtgaaccattgttcaatatcgtccggggacagcatcaaatcatccattgcttgggacggcaagggggactgtagatgggtgaaaagagcagtcagaggaccag

SEQ ID NO: 997 - DNA, 120 bp, human
  186573_9856668_TP53.1_1
cctccaggtccccagccctccaggtccccagcccaacccttgtccttaccagaacgttgttttcaggaagtctgaaagacaagagcagaaagtcagtcccatggaattttcgcttcccac

SEQ ID NO: 998 - DNA, 120 bp, human
  186573_9856669_TP53.1_1
cccttccaatggatccactcacagtttccataggtctgaaaatgtttcctgactcagagggggctcgacgctaggatctgactgcggctcctccatggcagtgacccggaaggcagtctg

SEQ ID NO: 999 - DNA, 120 bp, human
  186573_9856670_NFE2L2_1
ggatgtgctgggctggctgaattgggagaaattcacctgtctcttcatctagttgtaactgagcgaaaaaggctttctcttgctccttttggagttgttcttgtctttccttttcaagtt

SEQ ID NO: 1000 - DNA, 120 bp, human
  186573_9856670_NFE2L2_2
ttttctgtttttccagctcatactctttccgtcgctgactgaagtcaaatacttctcgacttactccaagatctatatcttgcctccaaagtatgtcaatcaaatccatgtcctatgttt

SEQ ID NO: 1001 - DNA, 120 bp, human
  186573_9856671_CASP8_1
acttagattatattctcctgccttttaaaaagatggacttcagcagaaatctttatgatattggggaacaactggacagtgaagatctggcctccctcaagttcctgagcctggactaca

SEQ ID NO: 1002 - DNA, 120 bp, human
  186573_9856671_CASP8_2
ttccgcaaaggaagcaagaacccatcaaggatgccttgatgttattccagagactccaggaaaagagaatgttggaggaaagcaatctgtccttcctgaaggagctgctcttccgaatta

SEQ ID NO: 1003 - DNA, 120 bp, human
  186573_9856671_CASP8_3
atagactggatttgctgattacctacctaaacactagaaaggaggagatggaaagggaacttcagacaccaggcagggctcaaatttctgcctacaggtgggtggaaactcccattgtgg

SEQ ID NO: 1004 - DNA, 120 bp, human
  186573_9856672_CASP8.1_1
tctgtgaattactgtggtataacgtgactgttcaaatttcacttttcaggggctttgaccacgacctttgaagagcttcattttgagatcaagccccacgatgactgcacagtagagcaa

SEQ ID NO: 1005 - DNA, 120 bp, human
  186573_9856672_CASP8.1_2
atctatgagattttgaaaatctaccaactcatggaccacagtaacatggactgcttcatctgctgtatcctctcccatggagacaagggcatcatctatggcactgatggacaggaggcc

SEQ ID NO: 1006 - DNA, 120 bp, human
  186573_9856672_CASP8.1_3
cccatctatgagctgacatctcagttcactggtttgaagtgcccttcccttgctggaaaacccaaagtgttttttattcaggcttgtcagggggataactaccagaaaggtatacctgtt

SEQ ID NO: 1007 - DNA, 120 bp, human
  186573_9856672_CASP8.1_4
gagactgattcagaggagcaaccctatttagaaatggatttatcatcacctcaaacgagatatatcccggatgaggctgactttctgctggggatggccactgtgaataactgtgtttcc

SEQ ID NO: 1008 - DNA, 120 bp, human
  186573_9856672_CASP8.1_5
taccgaaaccctgcagagggaacctggtacatccagtcactttgccagagcctgagagagcgatgtcctcggtaagttttgcctactcagccctcctcactgttacactaccttcccccc

SEQ ID NO: 1009 - DNA, 120 bp, human
  186573_9856673_CASP8.1_1
gcacagcagaggagacagttcatcagttgctttcccccacagacagtcacaatattatgtgatgtatttcagaggcgatgatattctcaccatcctgactgaagtgaactatgaagtaag

SEQ ID NO: 1010 - DNA, 119 bp, human
  186573_9856673_CASP8.1_2
caacaaggatgacaagaaaaacatggggaaacagatgcctcagcctactttcacactaagaaaaaaacttgtcttcccttctgattgatggtgctattttgtttgttttgttttgtttt

SEQ ID NO: 1011 - DNA, 120 bp, human
  186573_9856674_MAPK1_1
cttacctcgtcactcgggtcgtaatactgctccagatatgggtgggccagagcctgttctacttcaatcctcttgtgtgggttgaatgtcaacattttgtccaataagtccagagctata

SEQ ID NO: 1012 - DNA, 120 bp, human
  186573_9856675_EP300_1
taggagagtatacatatcttacctcgatagtgttcatttcttccgtcctaaatgcttgaggactgcagtctatcatgaaatcctaattggatatttagaatatgtcaagaaattagggta

SEQ ID NO: 1013 - DNA, 120 bp, human
  186573_9856676_PIK3CA_1
aacaatgcctccacgaccatcatcaggtgaactgtggggcatccacttgatgcccccaagaatcctagtagaatgtttactaccaaatggaatgatagtgactttagaatgcctccgtga

SEQ ID NO: 1014 - DNA, 120 bp, human
  186573_9856676_PIK3CA_2
ggctacattaataaccataaagcatgaactatttaaagaagcaagaaaataccccctccatcaacttcttcaagatgaatcttcttacattttcgtaagtgttactcaagaagcagaaag

SEQ ID NO: 1015 - DNA, 120 bp, human
  186573_9856676_PIK3CA_3
ggaagaattttttgatgaaacaagacgactttgtgaccttcggctttttcaaccctttttaaaagtaattgaaccagtaggcaaccgtgaagaaaagatcctcaatcgagaaattggtat

SEQ ID NO: 1016 - DNA, 120 bp, human
  186573_9856677_PIK3CA.1_1
gtaattttattaaaggttttgctatcggcatgccagtgtgtgaatttgatatggttaaagatccagaagtacaggacttccgaagaaatattctgaacgtttgtaaagaagctgtggatc

SEQ ID NO: 1017 - DNA, 120 bp, human
  186573_9856677_PIK3CA.1_2
ttagggacctcaattcacctcatagtagagcaatgtatgtctatcctccaaatgtagaatcttcaccagaattgccaaagcacatatataataaattagataaaggtaagaaaatgacta

SEQ ID NO: 1018 - DNA, 120 bp, human
  186573_9856678_PIK3CA.1_1
cccttgaaaaatgaaagagagatggtgattgcatctaatgttttcctgttatagggcaaataatagtggtgatctgggtaatagtttctccaaataatgacaagcagaagtatactctga

SEQ ID NO: 1019 - DNA, 120 bp, human
  186573_9856678_PIK3CA.1_2
aaatcaaccatgactgtgtaccagaacaagtaattgctgaagcaatcaggaaaaaaactcgaagtatgttgctatcctctgaacaactaaaactctgtgttttagaatatcagggcaagt

SEQ ID NO: 1020 - DNA, 120 bp, human
  186573_9856678_PIK3CA.1_3
atattttaaaagtgtgtggatgtgatgaatacttcctagaaaaatatcctctgagtcagtataaggtgagtaacaagtttcaaaatattaatttttaatttaaaaagtaatcacattgag

SEQ ID NO: 1021 - DNA, 120 bp, human
  186573_9856679_PIK3CA_1
ttacttttaaaatgaaaaaccttacaggaaatggctcgcccccttaatctcttacagtatataagaagctgtataatgcttgggaggatgcccaatttgatgttgatggctaaagaaagc

SEQ ID NO: 1022 - DNA, 120 bp, human
  186573_9856679_PIK3CA_2
ctttattctcaactgccaatggactgttttacaatgccatcttattccagacgcatttccacagctacaccatatatgaatggagaaacatctacaaaatccctttgggttataaatagt

SEQ ID NO: 1023 - DNA, 120 bp, human
  186573_9856679_PIK3CA_3
gcactcagaataaaaattctttgtgcaacctacgtgaatgtaaatattcgagacattgataaggtaaagtcaaatgctgatgcttattattttatagaaattattttagataaccttttt

SEQ ID NO: 1024 - DNA, 120 bp, human
  186573_9856680_PIK3CA.1_1
ctacttttttcttttagatctatgttcgaacaggtatctaccatggaggagaacccttatgtgacaatgtgaacactcaaagagtaccttgttccaatcccaggtaaggaagtatataga

SEQ ID NO: 1025 - DNA, 120 bp, human
  186573_9856681_PIK3CA.1_1
tacataggtggaatgaatggctgaattatgatatatacattcctgatcttcctcgtgctgctcgactttgcctttccatttgctctgttaaaggccgaaagggtgctaaagaggtaaagt

SEQ ID NO: 1026 - DNA, 120 bp, human
  186573_9856682_PIK3CA_1
gttttataatttagactagtgaatatttttctttgttttttaaggaacactgtccattggcatggggaaatataaacttgtttgattacacagacactctagtatctggaaaaatggctt

SEQ ID NO: 1027 - DNA, 120 bp, human
  186573_9856682_PIK3CA_2
tgaatctttggccagtacctcatggattagaagatttgctgaaccctattggtgttactggatcaaatccaaataaagtaaggtttttattgtcataaattagatattttttatggcagt

SEQ ID NO: 1028 - DNA, 120 bp, human
  186573_9856683_PIK3CA_1
agtcaaaccttctctcttatgtatatataatagcttttcttccatctcttaggaaactccatgcttagagttggagtttgactggttcagcagtgtggtaaagttcccagatatgtcagt

SEQ ID NO: 1029 - DNA, 120 bp, human
  186573_9856683_PIK3CA_2
gattgaagagcatgccaattggtctgtatcccgagaagcaggatttagctattcccacgcaggactggtaaggcaaatcactgagtttattaagtatcaattataatctgtggatttagg

SEQ ID NO: 1030 - DNA, 120 bp, human
  186573_9856684_PIK3CA_1
atccagaggggaaaaatatgacaaagaaagctatataagatattattttattttacagagtaacagactagctagagacaatgaattaagggaaaatgacaaagaacagctcaaagcaat

SEQ ID NO: 1031 - DNA, 120 bp, human
  186573_9856684_PIK3CA_2
ttctacacgagatcctctctctgaaatcactgagcaggagaaagattttctatggagtcacaggtaagtgctaaaatggagattctctgtttctttttctttattacagaaaaaataact

SEQ ID NO: 1032 - DNA, 120 bp, human
  186573_9856685_PIK3CA_1
ttttgtttctcccacacagacactattgtgtaactatccccgaaattctacccaaattgcttctgtctgttaaatggaattctagagatgaagtagcccaggtaaatgtatgtttgagat

SEQ ID NO: 1033 - DNA, 120 bp, human
  186573_9856686_PIK3CA_1
cccctttaaatatgatttattgtctttctcatacacagatgtattgcttggtaaaagattggcctccaatcaaacctgaacaggctatggaacttctggactgtaattacccagatccta

SEQ ID NO: 1034 - DNA, 120 bp, human
  186573_9856686_PIK3CA_2
tggttcgaggttttgctgttcggtgcttggaaaaatatttaacagatgacaaactttctcagtatttaattcagctagtacaggtaaaataatgtaaaatagtaaataatgtttaattac

SEQ ID NO: 1035 - DNA, 120 bp, human
  186573_9856687_PIK3CA_1
tcacctaggtcctaaaatatgaacaatatttggataacttgcttgtgagatttttactgaagaaagcattgactaatcaaaggattgggcactttttcttttggcatttaaagtaagtct

SEQ ID NO: 1036 - DNA, 120 bp, human
  186573_9856688_PIK3CA.1_1
tatatatatttttaattttgcacgattcttttagatctgagatgcacaataaaacagttagccagaggtttggcctgcttttggagtcctattgtcgtgcatgtgggatgtatttgaagc

SEQ ID NO: 1037 - DNA, 120 bp, human
  186573_9856688_PIK3CA.1_2
acctgaataggcaagtcgaggcaatggaaaagctcattaacttaactgacattctcaaacaggagaagaaggatgaaacacaaaaggtgtgtgactctagtttgtgtttgagactctttt

SEQ ID NO: 1038 - DNA, 120 bp, human
  186573_9856689_PIK3CA_1
aatcaggtacagatgaagtttttagttgagcaaatgaggcgaccagatttcatggatgctctacagggctttctgtctcctctaaaccctgctcatcaactaggaaacctcaggtacttt

SEQ ID NO: 1039 - DNA, 120 bp, human
  186573_9856690_PIK3CA_1
ttcagggtaaaataataataaagcaaaggtacctagtaaagtttttaactattttaaaggcttgaagagtgtcgaattatgtcctctgcaaaaaggccactgtggttgaattgggagaac

SEQ ID NO: 1040 - DNA, 120 bp, human
  186573_9856690_PIK3CA_2
ccagacatcatgtcagagttactgtttcagaacaatgagatcatctttaaaaatggggatggtaaggaagagtattaatgagcttatgatgcatgaatttagctatctttttatacacag

SEQ ID NO: 1041 - DNA, 120 bp, human
  186573_9856691_PIK3CA_1
acatattatttgaatttcagatttacggcaagatatgctaacacttcaaattattcgtattatggaaaatatctggcaaaatcaaggtcttgatcttcggtaggtaaccagtaaggcaac

SEQ ID NO: 1042 - DNA, 120 bp, human
  186573_9856692_PIK3CA_1
tcatttaattgtaaacgtgttactcctctttcagaatgttaccttatggttgtctgtcaatcggtgactgtgtgggacttattgaggtggtgcgaaattctcacactattatgcaaattc

SEQ ID NO: 1043 - DNA, 120 bp, human
  186573_9856692_PIK3CA_2
agtgcaaaggcggcttgaaaggtgcactgcagttcaacagccacacactacatcagtggctcaaagacaagaacaaaggagaaatgtgagttgtattattctttcttcctatgttaatct

SEQ ID NO: 1044 - DNA, 120 bp, human
  186573_9856693_PIK3CA_1
gatatgatgcagccattgacctgtttacacgttcatgtgctggatactgtgtagctaccttcattttgggaattggagatcgtcacaatagtaacatcatggtgaaagacgatggacaag

SEQ ID NO: 1045 - DNA, 120 bp, human
  186573_9856694_PIK3CA.1_1
atctgcatatatcaaactataacataatttcttatttttgaaagctgtttcatatagattttggacactttttggatcacaagaagaaaaaatttggttataaacgagaacgtgtgccat

SEQ ID NO: 1046 - DNA, 120 bp, human
  186573_9856694_PIK3CA.1_2
ttgttttgacacaggatttcttaatagtgattagtaaaggagcccaagaatgcacaaagacaagagaatttgagaggtgagctcgagcaattaaaaacacaaaataaagagttctggctg

SEQ ID NO: 1047 - DNA, 120 bp, human
  186573_9856695_PIK3CA.1_1
acatcatttgctccaaactgaccaaactgttcttattacttataggtttcaggagatgtgttacaaggcttatctagctattcgacagcatgccaatctcttcataaatcttttctcaat

SEQ ID NO: 1048 - DNA, 120 bp, human
  186573_9856695_PIK3CA.1_2
gatgcttggctctggaatgccagaactacaatcttttgatgacattgcatacattcgaaagaccctagccttagataaaactgagcaagaggctttggagtatttcatgaaacaaatgaa

SEQ ID NO: 1049 - DNA, 120 bp, human
  186573_9856695_PIK3CA.1_3
tgatgcacatcatggtggctggacaacaaaaatggattggatcttccacacaattaaacagcatgcattgaactgaaaagataactgagaaaatgaaagctcactctggattccacactg

SEQ ID NO: 1050 - DNA, 120 bp, human
  186573_9856696_FBXW7.1_1
atcttacctgtaatgaatagactctattagtatgcccctgcaacgtgtgtagacaggtttcagtctctggatcccacacctttaccataaaatcatatgctccactaacaaccctcctgc

SEQ ID NO: 1051 - DNA, 120 bp, human
  186573_9856696_FBXW7.1_2
catcatattgaacacagcggactgctgcaacatgacccatcaaaacatgtaaacactggcctgtctcaatatcccaaaccctaagagtggcatctcgagaaccgctaacaactctgcaga

SEQ ID NO: 1052 - DNA, 120 bp, human
  186573_9856697_TERT_1
ctgcgcagcagggagcgcacggctcggcagcggggagcgcgcggcatcgcgggggtggccggggccagggcttcccacgtgcgcagcaggacgcagcgctgcctgaaactcgcgccgcga

SEQ ID NO: 1053 - DNA, 120 bp, human
  186573_9856697_TERT_2
ggagagggcggggccgcggaaaggaaggggaggggctgggagggcccggagggggctgggccggggacccgggaggggtcgggacggggcggggtccgcgcggaggaggcggagctggaa

SEQ ID NO: 1054 - DNA, 120 bp, human
  186573_9856698_CDKN2A_1
acaaattctcagatcatcagtcctcacctgagggaccttccgcggcatctatgcgggcatggttactgcctctggtgccccccgcagccgcgcgcaggtaccgtgcgacatcgcgatggc

SEQ ID NO: 1055 - DNA, 120 bp, human
  186573_9856698_CDKN2A_2
ccagctcctcagccaggtccacgggcagacggccccaggcatcgcgcacgtccagccgcgccccggcccggtgcagcaccaccagcgtgtccaggaagccctcccgggcagcgtcgtgca

SEQ ID NO: 1056 - DNA, 120 bp, human
  186573_9856698_CDKN2A_3
cgggtcgggtgagagtggcggggtcggcgcagttgggctccgcgccgtggagcagcagcagctccgccactcgggcgctgcccatcatcatgacctgccagagagaacagaatggtcaga

SEQ ID NO: 1057 - DNA, 120 bp, human
  186573_9856699_CDKN2A_1
gagtcgcccgccatcccctgctcccgctgcagaccctctacccacctggatcggcctccgaccgtaactattcggtgcgttgggcagcgcccccgcctccagcagcgcccgcacctcctc

SEQ ID NO: 1058 - DNA, 120 bp, human
  186573_9856699_CDKN2A_2
tacccgaccccgggccgcggccgtggccagccagtcagccgaaggctccatgctgctccccgccgccggctccatgctgctccccgccgcccgctgcctgctctccccctctccgcagcc

SEQ ID NO: 1059 - DNA, 120 bp, human
  186573_9856700_CDKN2A_1
ggacttttcgagggcctttcctacctggtcttctaggaagcggctgctgccctagacgctggctcctcagtagcatcagcacgagggccacagcggcgggcgcccctggcgctgcccact

SEQ ID NO: 1060 - DNA, 120 bp, human
  186573_9856700_CDKN2A_2
cccccgtgagccgcgggatgtgaaccacgaaaaccctcactcgcggcgggccgcacgcgcgccgaatccggagggtcaccaagaacctgcgcaccatgttctcgccgcctccagggccga

SEQ ID NO: 1061 - DNA, 120 bp, human
  186573_9856701_NOTCH1_1
ggtttcccgccctggccccggccgacgcaccgggcatgcagatgcactggaactccccaatctggtccaggcaggtggcgtcgttctggcacgggttcgagacgcactcgttgacgtcga

SEQ ID NO: 1062 - DNA, 120 bp, human
  186573_9856701_NOTCH1_2
tctcgcatcgggggcccgtgtagccctgcagacactggcactcgaaggagcccagcgtgttgatgcacttgcccgcatgctcgcaggggttggcacctggcgagggcacacgggtgagag

SEQ ID NO: 1063 - DNA, 120 bp, human
  186573_9856702_NOTCH1_1
gcggccatggggacactcgcagtagaaggaggccacacggtcatggcaggtggcgccgtggaagcaggcggcgctggcacagtcatcaatgttctcgctgcagtcctcaccagtccagcc

SEQ ID NO: 1064 - DNA, 120 bp, human
  186573_9856702_NOTCH1_2
gttgacacacacgcagttgtagccaccgtgggtgttgtggcaggtcccgccgttctggcaggcatttggcatcagctggcactcgtccacatcctcggtacagtactgacctgcagggaa